(19) United States
(12) Patent Application Publication
(10) Pub. No.: US 2023/0078913 A1
(43) Pub. Date: Mar. 16, 2023

(54) ANDROGEN RECEPTOR MODULATORS AND METHODS FOR USE AS PROTEOLYSIS TARGETING CHIMERA LIGANDS

(71) Applicants: ESSA Pharma, Inc., Vancouver (CA); The University of British Columbia, Vancouver (CA)

(72) Inventors: Han-Jie Zhou, Foster City, CA (US); Raymond John Andersen, Vancouver (CA)

(21) Appl. No.: 17/599,341

(22) PCT Filed: Mar. 27, 2020
(86) PCT No.: PCT/US2020/025542

§ 371 Date: Sep. 28, 2021

Related U.S. Application Data
(60) Provisional application No. 62/825,387, filed on Mar. 28, 2019.

(51) Int. Cl. A61K 47/55; A61K 47/54; C07D 239/42

(52) U.S. Cl. CPC A61K 47/55; C07D 239/42; A61K 47/54

(57) ABSTRACT

The present invention relates to bifunctional Proteolysis Targeting Chimeric ligands (Protac compounds) comprising a ligase modulator/binder and a molecule that binds to a protein target of interest, and methods of treating various diseases and conditions with the Protac compounds, including diseases associated with androgen receptors.

### CROSS-REFERENCE TO RELATED APPLICATIONS

The application claims priority to U.S. Provisional Application No. 62/825,387, filed Mar. 28, 2019, the disclosures of which are hereby incorporated by reference in their entireties for all purposes.

### FIELD OF THE INVENTION

The present disclosure generally relates to bifunctional Proteolysis Targeting Chimeric ligands (Protac compounds) comprising a ligase modulator/binder and a molecule that binds to a protein target of interest, and methods of treating various diseases and conditions with the Protac compounds. Generally, the molecule that binds to a protein target is an androgen receptor modulator.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration (androgen ablation), also known as androgen ablation therapy (ABT) or androgen depravation therapy (ADT).

Androgens also play a role in female diseases such as polycystic ovary syndrome as well as cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate luminal cells. Androgen ablation therapy causes a temporary reduction in tumor burden concomitant with a decrease in serum prostate-specific antigen (PSA). Unfortunately prostate cancer can eventually grow again in the absence of testicular androgens (castration-resistant disease) (Huber et al 1987 Scand J. Urol Nephrol. 104, 33-39). Castration-resistant prostate cancer that is still driven by AR is biochemically characterized before the onset of symptoms by a rising titre of serum PSA (Miller et al 1992 J Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains two transcriptional activation units (tau1 and tau5) within activation function-1 (AF-1). Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J Biol. Chem. 274, 7777-7783; Ueda et al 2002 A J Biol. Chem. 277, 7076-7085; and Ueda et al 2002 B J Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J. Biol. Chem. 277, 7076-7085; and Ueda et al 2002 B J Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J Pathol. 160, 219-226; and van der Kwast et al 1991 Inter. J Cancer 48, 189-193).

Clinically available inhibitors of the AR include nonsteroidal antiandrogens such as bicalutamide (Casodex™), nilutamide, flutamide, and enzalutamide. There is also a class of steroidal antiandrogens, such as cyproterone acetate and spironolactone. Both steroidal and non-steroidal antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)), and constitutively active AR splice variants. Antiandrogens have no effect on the constitutively active AR splice variants that lack the ligand-binding domain (LBD) and are associated with castration-recurrent prostate cancer (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30) and resistant to abiraterone and enzalutamide (Antonarakis et al., N Engl J Med. 2014, 371, 1028-38; Scher et al JAMA Oncol. 2016 doi: 10.1001). Conventional therapy has concentrated on androgen-dependent activation of the AR through its C-terminal domain.

Other relevant AR antagonists previously reported (see, WO 2010/000066, WO 2011/082487; WO 2011/082488; WO 2012/145330; WO 2015/031984; WO 2016/058080; and WO 2016/058082) that bind to full-length AR and/or truncated AR splice variants that are currently being developed include: AR degraders such as niclosamide (Liu C et al 2014), galeterone (Njar et al 2015; Yu Z at al 2014), and ARV-330/Androgen receptor PROTAC (Neklesa et al 2016 J Clin Oncol 34 suppl 2S; abstr 267); AR DBD inhibitor VPC-14449 (Dalal K et al 2014 J Biol Chem. 289(38):26417-29; Li H et al 2014 J Med Chem. 57(15):6458-67); antiandrogens apalutamide (Clegg N J et al 2012), ODM-201 (Moilanen A M et al 2015), ODM-204 (Kallio et al J Clin Oncol 2016 vol. 34 no. 2_suppl 230), TAS3681 (Minamiguchi et al 2015 J Clin Oncol 33, suppl 7; abstr 266); and AR NTD inhibitors 3E10-AR441bsAb (Goicochea N L et al 2015), and sintokamide (Sadar et al 2008; Banuelos et al 2016).

The AR-NTD is also a target for drug development (e.g. WO 2000/001813; Myung et al. J. Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J Biol. Chem. 274, 7777-7783; Sadar M D et al 1999 Endocr Relat Cancer. 6, 487-502; Ueda et al 2002 J Biol. Chem. 277, 7076-7085; Ueda 2002 J Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches. Compounds that modulate AR, potentially through interaction with NTD domain, include the bisphenol compounds disclosed in published PCT Nos: WO 2010/000066, WO 2011/082487; WO 2011/082488; WO 2012/145330; WO 2012/139039; WO 2012/145328; WO 2013/028572; WO 2013/028791; WO 2014/179867; WO 2015/031984; WO 2016/058080; WO 2016/058082; WO 2016/112455; WO 2016/141458; WO 2017/177307; WO 2017/210771; and WO 2018/045450, and which are hereby incorporated by reference in their entireties.

Transcriptionally active androgen receptor plays a major role in CRPC in spite of reduced blood levels of androgen (Karantanos, T. et al Oncogene 2013, 32, 5501-5511; Harris, W. P. et al Nature Clinical Practice Urology, 2009, 6, 76-85). AR mechanisms of resistance to ADT include: overexpression of AR (Visakorpi, T. et al Nature Genetics 1995, 9, 401-406; Koivisto, P. et al Scandinavian Journal of Clinical and Laboratory Investigation Supplementum 1996, 226, 57-63); gain-of-function mutations in the AR LBD (Culig Z. et al Molecular Endocrinology 1993, 7, 1541-1550); intratumoral androgen synthesis (Cai, C. et al Cancer Research 2011, 71, 6503-6513); altered expression and function of AR coactivators (Ueda, T. et al The Journal of Biological Chemistry 2002, 277, 38087-38094; Xu J. et al Nature Reviews Cancer 2009, 9, 615-630); aberrant post-translational modifications of AR (Gioeli D. et al Molecular and Cellular Endocrinology 2012, 352, 70-78; van der Steen T. et al International Journal of Molecular Sciences 2013, 14, 14833-14859); and expression of AR splice variants (AR-Vs) which lack the ligand-binding domain (LBD) (Karantanos, T. et al Oncogene 2013, 32, 5501-5511; Andersen R. J. et al Cancer Cell 2010, 17, 535-546; Myung J. K. et al The Journal of Clinical Investigation 2013, 123, 2948-2960; Sun S. et al The Journal of Clinical Investigation 2010, 120, 2715-2730). Anti-androgens such as bicalutamide and enzalutamide target AR LBD, but have no effect on truncated constitutively active AR-Vs such as AR-V7 (Li Y. et al Cancer Research 2013, 73, 483-489). Expression of AR-V7 is associated with resistance to current hormone therapies (Li Y. et al Cancer Research 2013, 73, 483-489; Antonarakis E. S. et al The New England Journal of Medicine 2014, 371, 1028-1038).

While significant advances have been made in this field, there remains a need for improved treatment for AR-mediated disorders including prostate cancer, especially metastatic castration-resistant prostate cancer. Development of compounds and complexes that can selectively act to inhibit AR activity or degrade AR proteins that promotes cell proliferation, via unique interactions with AR NTD, would provide patients alternative options and new hope.

Ubiquitin-Proteasome Pathway System (UPS) is a critical pathway that regulates key regulator proteins and degrades misfolded or abnormal proteins. UPS is central to multiple cellular processes, and if defective or imbalanced, it leads to pathogenesis of a variety of diseases. Posttranslational modification of proteins by ubiquitin is a fundamental cellular mechanism that regulates protein stability and activity and underlies a multitude of functions, from almost every aspect of biology. The covalent attachment of ubiquitin to specific protein substrates is achieved through the action of E3 ubiquitin ligases. These ligases comprise over 500 different proteins and are categorized into multiple classes defined by the structural element of their E3 functional activity.

Deubiquitinating proteins and ubiquitin-specific proteases (DUBs and USPs) and E3 Ligases play a vital role in the UPS. These proteins are supported by flexible Zinc Finger (ZnF) domains which stabilize the binding of ubiquitin (Ub) for specialized functions.

The present invention relates to bifunctional compounds, also known as Proteolysis Targeting Chimeric molecules (Protac) that induce ubiquitination and degrade a protein of interest. Protac compounds are typically designed with three parts: 1) a ligand/molecule that binds to and/or modulates ubiquitin ligases; 2) a small molecule that binds to the target protein of interest for proteolysis; and 3) a linker that links the two molecules together. Protacs thus function by allowing the ligand/molecule to bind to the ubiquitin ligases, thereby recruiting the target of protein of interest to the ligase for ubiquitination and ultimately proteolysis and degradation.

The present invention discloses Protac compounds intended to degrade and/or inhibit AR proteins associated with cancer, especially prostate cancer.

### SUMMARY OF THE INVENTION

The compound of the present disclosure can be useful for modifying the ubiquitination and subsequent degradation of androgen receptor proteins. In one embodiment of the present invention, the compound is a bifunctional compound wherein a E3 ligase binding group (“PLM”) is covalently attached to one end of a Linker (“LI”), and the androgen receptor modulatr (“PTC”) is covalently attached to the other end of the linker (LI).

In one embodiment, the compound of the present disclosure is represented by formula (Q):

PLM-LI-PTC (Q);

or a pharmaceutically acceptable salt thereof, wherein:
PLM is a E3 ligase binding group,
LI is a linker, and
PTC is an androgen receptor modulator represented by formula (IIIA):

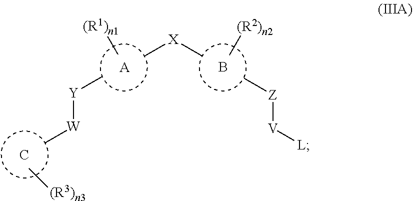

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

In some embodiments of the compound of formula (Q), the linker LI corresponds to the formula:

-L_{I}-L_{II}(q)-,

wherein:
L_{I} is a bond or a chemical group coupled to at least one of a PLM, a PTC or a combination thereof,
L_{II} is a bond or a chemical group coupled to at least one of a PLM, a PTC,
and q is an integer greater than or equal to 0;
wherein each L_{I} and L_{II} is independently selected from a bond, CR^{L1}R^{L2}, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, NR^{L3} SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁ cycloalkyl optionally substituted with 0-6 R^{L1} and/or R^{L2} groups, C₃₋₁₁ heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups;
wherein i is 1, 2, 3, 4, 5, 6, 7, 8, 9 or 10; and
wherein R^{L1}, R^{L2}, R^{L3}, R^{L4} and R^{L5} are, each independently, H, halo, —C₁₋₈ alkyl, —OC₁₋₈ alkyl, —SC₁₋₈ alkyl, —NHC₁₋₈ alkyl, —N(C₁₋₈ alkyl)₂, —C₃₋₁₁ cycloalkyl, aryl, heteroaryl, —C₃₋₁₁ heterocyclyl, —OC₁₋₈ cycloalkyl, —SC₁₋₈ cycloalkyl, —NHC₁₋₈ cycloalkyl, —N(C₁₋₈ cycloalkyl)₂, —N(C₁₋₈ cycloalkyl)(C₁₋₈ alkyl), —OH, —NH₂, —SH, —SO₂C₁₋₈ alkyl, —P(O)(OC₁₋₈ alkyl)(C₁₋₈ alkyl), —P(O)(OC₁₋₈ alkyl)₂, —C≡C—C₁₋₈ alkyl, —CCH, —CH═CH(C₁₋₈ alkyl), —C(C₁₋₈ alkyl)=CH(C₁₋₈ alkyl), —C(C₁₋₈ alkyl)=C(C₁₋₈ alkyl)₂, —Si(OH)₃, —Si(C₁₋₈ alkyl)₃, —Si(OH)(C₁₋₈ alkyl)₂, —C(═O)C₁₋₈ alkyl, —CO₂H, halogen, —CN, —CF₃, —CHF₂, —CH₂F, —NO₂, —SF₅, —SO₂NHC₁₋₈ alkyl, —SO₂N(C₁₋₈ alkyl)₂, —SONHC₁₋₈ alkyl, —SON(C₁₋₈ alkyl)₂, —CONHC₁₋₈ alkyl, —CON(C₁₋₈ alkyl)₂, —N(C₁₋₈ alkyl)CONH(C₁₋₈ alkyl), —N(C₁₋₈ alkyl)CON(C₁₋₈ alkyl)₂, —NHCONH(C₁₋₈ alkyl), —NHCON(C₁₋₈ alkyl)₂, —NHCONH₂, —N(C₁₋₈ alkyl)SO₂NH(C₁₋₈ alkyl), —N(C₁₋₈ alkyl)SO₂N(C₁₋₈ alkyl)₂, —NHSO₂NH(C₁₋₈ alkyl), —NHSO₂N(C₁₋₈ alkyl)₂, or —NHSO₂NH₂.

In some embodiments of the compound of formula (Q), q is selected from 1, 2, 3, 4, 5, 6, 7, 8, 9, 10, 11, 12, 13, 14, 15, 16, 17, 18, 19, 20, 21, 22, 23, or 24.

In some embodiments of the compound of formula Q, the PLM is a von Hippel-Lindau (VHL) binding group, an E3 ligase substrate receptor cereblon (CRBN), a mouse double minute 2 homolog (MDM2), or an inhibitor of apoptosis (IAP). In some embodiments, the PLM is a von Hippel-Lindau (VHL) binding group.

In some embodiments of the compound of formula (Q), the PLM has the formula (E3B):

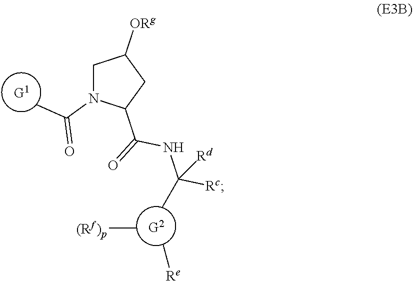

wherein, G¹ is optionally substituted aryl, optionally substituted heteroaryl, or —CR⁹R¹⁰R¹¹;
each R⁹ and R¹⁰ is independently hydrogen, optionally substituted alkyl, optionally substituted cycloalkyl, optionally substituted hydroxyalkyl, optionally substituted heteroaryl, or haloalkyl; or R⁹ and R¹⁰ and the carbon atom to which they are attached form an optionally substituted cycloalkyl;
R¹¹ is optionally substituted heterocyclic, optionally substituted alkoxy, optionally substituted heteroaryl, optionally substituted aryl, or —NR¹²R¹³,

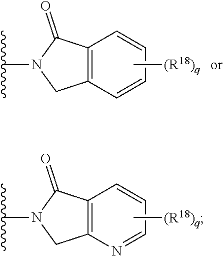

R¹² is H or optionally substituted alkyl;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} and R^{d} is each independently H, haloalkyl, or optionally substituted alkyl;
G² is a phenyl or a 5-10 membered heteroaryl,
R^{e} is H, halogen, CN, OH, NO₂, NR^{c}R^{d}, OR^{c}R, CONR^{c}R^{d}, NR^{c}COR^{d}, SO₂NR^{c}R^{d}, NR^{c}SO₂R^{d}, optionally substituted alkyl, optionally substituted haloalkyl, optionally substituted haloalkoxy; optionally substituted aryl; optionally substituted heteroaryl; optionally substituted cycloalkyl; optionally substituted cycloheteroalkyl;
each R^{f} is independently halo, optionally substituted alkyl, haloalkyl, hydroxy, optionally substituted alkoxy, or haloalkoxy;
R^{g} is H, C₁₋₆ alkyl, —C(O)R¹⁹; —C(O)OR¹⁹; or —C(O)NR¹⁹R¹⁹;
p is 0, 1, 2, 3, or 4;
each R¹⁸ is independently halo, optionally substituted alkoxy, cyano, optionally substituted alkyl, haloalkyl, haloalkoxy or a linker;
each R¹⁹ is independently H, optionally substituted alkyl, or optionally substituted aryl;
q is 0, 1, 2, 3, or 4; and
wherein any one of the hydrogen atoms in the PLM can be replaced to form a covalent bond to the LI.

In some embodiments of the compound of formula (Q), the PLM has the formula (E3D):

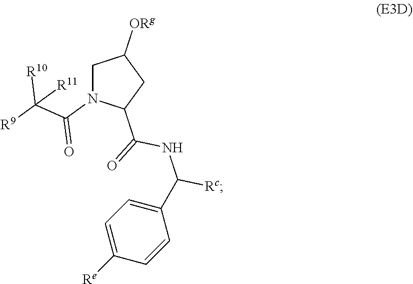

wherein any one of the hydrogen atoms in the PLM can be replaced to form a covalent bond to the LI.

In some embodiments of the compound of formula (Q), the PLM is represented by formula (W-II):

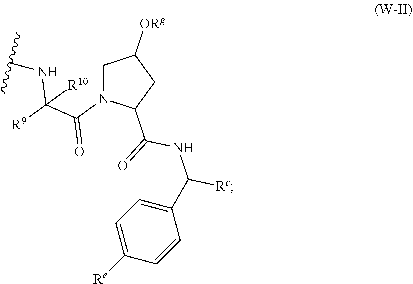

wherein the PLM is covalently bound to the LI via

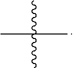

In some embodiments of the compound of formula (Q), the PLM is represented by formula (W-IIIA):

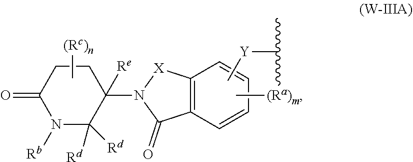

or an enantiomer, diastereomer, stereoisomer, or a pharmaceutically acceptable salt thereof, wherein:
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently halogen, OH, C₁₋₆ alkyl, or C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O), a C₃-C₆ carbocycle, or a 4- to 6-membered heterocycle comprising 1 or 2 heteroatoms selected from N or O;
R^{e} is H, deuterium, C₁₋₃ alkyl, F, or Cl;
m is 0, 1, 2 or 3;
n is 0, 1 or 2; and
wherein the PLM is covalently bound to the LI via

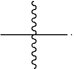

In some embodiments of the compound of formula (Q), the PLM is represented by formula (W-IIIB):

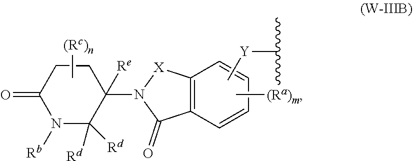

or an enantiomer, diastereomer, stereoisomer, or a pharmaceutically acceptable salt thereof, wherein the PLM is covalently bound to the LI via

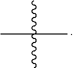

In some embodiments of the compound of formula (Q), the PTC has the structure of formula (IVA):

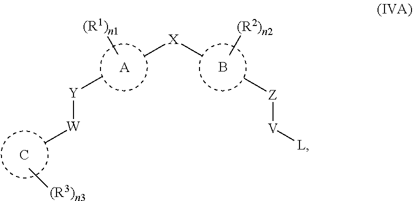

or a pharmaceutically acceptable salt, tautomer, stereoisomer or prodrug thereof.

In some embodiments of the compound of formula (Q), the PTC has the structure of formula (A-I)

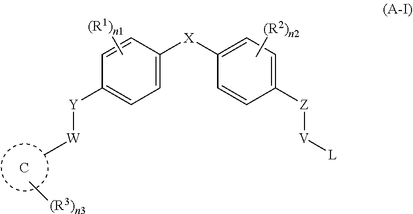

or a pharmaceutically acceptable salt, tautomer, stereoisomer or prodrug thereof.

In some embodiments of the compound of formula (Q), the PTC has the structure of formula (G-II):

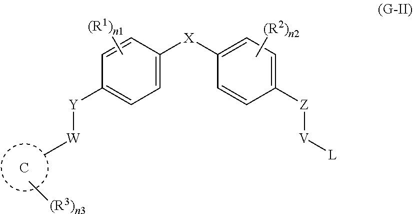

or a pharmaceutically acceptable salt, tautomer, stereoisomer or prodrug thereof.

In some embodiments, the compound of formula (Q) is a compound of formula (W-IV):

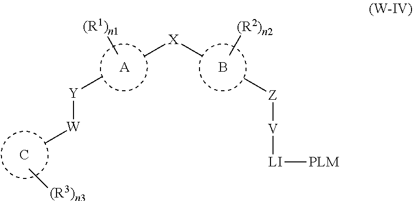

or a pharmaceutically acceptable salt thereof.

In some embodiments, the compound of formula (Q) is a compound of formula (W-IVA):

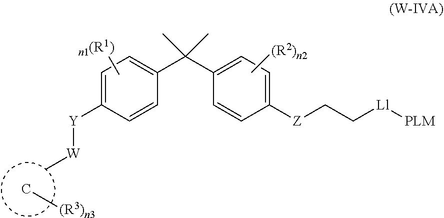

or a pharmaceutically acceptable salt thereof.

In some embodiments, the compound of formula (Q) is a compound of formula (W-V):

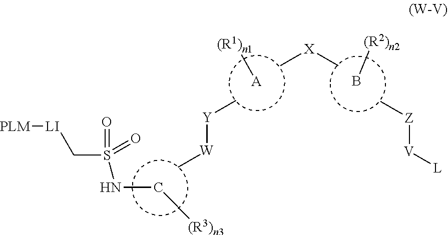

or a pharmaceutically acceptable salt thereof.

In some embodiments, the compound of formula (Q) is a compound of formula (W-VA):

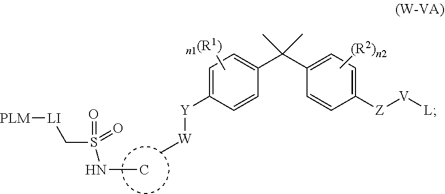

or a pharmaceutically acceptable salt thereof.

In some embodiments, the compound of formula (Q) is a compound of formula (W-VI):

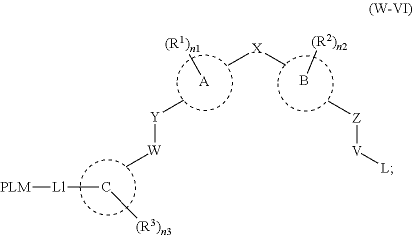

or a pharmaceutically acceptable salt thereof.

In some embodiments, the compound of formula (Q) is a compound of formula (W-VIA):

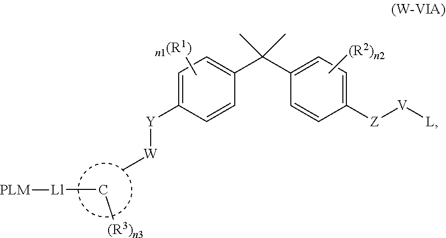

or a pharmaceutically acceptable salt thereof.

In some embodiments, the compound of formula (Q) is a compound of formula (W-VII):

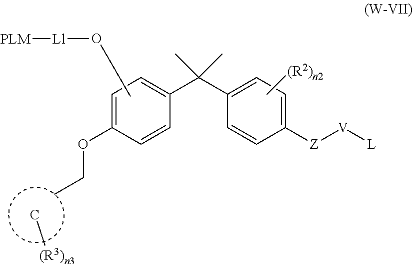

or a pharmaceutically acceptable salt thereof.

In one embodiment of the present disclosure, a pharmaceutical composition comprising a compound of formula (Q) and a pharmaceutically acceptable carrier is provided.

In one embodiment of the pharmaceutical composition as disclosed herein, the composition further comprising one or more additional therapeutic agents.

In one embodiment, the present disclosure relates to methods for modulating androgen receptor activity, comprising administering a compound of formula (Q), to a subject in need thereof.

In one embodiment of any one of the method as disclosed herein, the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

In one embodiment, the present disclosure relates to methods for treating cancer, comprising administering a compound of formula (Q), to a subject in need thereof. In one embodiment, the cancer is selected from prostate cancer, breast cancer, ovarian cancer, bladder cancer, pancreatic cancer, hepatocellular cancer, endometrial cancer, or salivary gland carcinoma. In one embodiment, the cancer is prostate cancer.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo. In one embodiment of the present disclosure, a prodrug includes a PTC of formula (I), (IA), (IB), (IC), (II), (IIA), (IIIA), (IIB), (III), (IV), (IVA), (V), (VA), (VI), (A), (A-I), (B)-(D), (E), (E-I)-(E-VII), (F), (G), (G-I), (G-II), (H), and (H-I) (“formula (I)-(VI) and (A)-(H-I)”) and (a), having a free hydroxyl group (—OH) that is acetylated (—OCOMe) at one or more positions.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof

“Ubiquitin Proteasome Pathway System (UPS)” as used herein relates to the ubiquitin proteasome pathway, conserved from yeast to mammals, and is required for the targeted degradation of most short-lived proteins in the eukaryotic cell. Targets include cell cycle regulatory proteins, whose timely destruction is vital for controlled cell division, as well as proteins unable to fold properly within the endoplasmic reticulum. Ubiquitin modification is an ATP-dependent process carried out by three classes of enzymes. An “ubiquitin activating enzyme” (E1) forms a thio-ester bond with ubiquitin, a highly conserved 76-amino acid protein. This reaction allows subsequent binding of ubiquitin to a “ubiquitin conjugating enzyme” (E2), followed by the formation of an isopeptide bond between the carboxy-terminus of ubiquitin and a lysine residue on the substrate protein. The latter reaction requires a “ubiquitin ligase” (E3). E3 ligases can be single- or multi-subunit enzymes. In some cases, the ubiquitin-binding and substrate binding domains reside on separate polypeptides brought together by adaptor proteins or culling. Numerous E3 ligases provide specificity in that each can modify only a subset of substrate proteins. Further specificity is achieved by post-translational modification of substrate proteins, including, but not limited to, phosphorylation. Effects of monoubiquitination include changes in subcellular localization. However, multiple ubiquitination cycles resulting in a polyubiquitin chain are required for targeting a protein to the proteasome for degradation. The multisubunit 26S proteasome recognizes, unfolds, and degrades polyubiquitinated substrates into small peptides. The reaction occurs within the cylindrical core of the proteasome complex, and peptide bond hydrolysis employs a core threonine residue as the catalytic nucleophile. It has been shown that an additional layer of complexity, in the form of multiubiquitin chain receptors, may lie between the polyubiquitination and degradation steps. These receptors react with a subset of polyubiquitinated substrates, aiding in their recognition by the 26S proteasome, and thereby promoting their degradation. This pathway is not only important in cellular homeostasis, but also in human disease. Because ubiquitin/proteasome-dependent degradation is often employed in control of the cell division cycle and cell growth, researchers have found that proteasome inhibitors hold some promise of being developed into potential cancer therapeutic agents.

Protein degradation through the ubiquitin-proteasome system is the major pathway of non-lysosomal proteolysis of intracellular proteins. It plays important roles in a variety of fundamental cellular processes such as regulation of cell cycle progression, division, development and differentiation, apoptosis, cell trafficking, and modulation of the immune and inflammatory responses. The central element of this system is the covalent linkage of ubiquitin to targeted proteins, which are then recognized by the 26S proteasome, an adenosine triphosphate-dependent, multi-catalytic protease. Damaged, oxidized, or misfolded proteins as well as regulatory proteins that control many critical cellular functions are among the targets of this degradation process. Aberration of this system leads to the dysregulation of cellular homeostasis and the development of multiple diseases (Wang et al. Cell Mol Immunol. 2006 August; 3(4):255-61).

“Ligase” as used herein, is an enzyme that can catalyze the joining of two or more compounds or biomolecules by bonding them together with a new chemical bond. The “ligation” of the two usually with accompanying hydrolysis of a small chemical group dependent to one of the larger compounds or biomolecules, or the enzyme catalyzing the linking together of two compounds, e.g., enzymes that catalyze joining of groups C—O, C—S, C—N, etc. Ubiquitin-protein (E3) ligases are a large family of highly diverse enzymes selecting proteins for ubiquitination.

“Ub Ligases” are involved in disease pathogenesis for oncology, inflammation & infectious disease. E3 ligase belonging to the RING-between-RING (RBR) family of E3 ligases containing both canonical RING domains and a catalytic cysteine residue usually restricted to HECT E3 ligases; termed ‘RING/HECT hybrid’ enzymes. Mutations in Parkin linked to Parkinson's disease, cancer and mycobacterial infection. Parkin is recognized as a neuroprotective protein with a role in mitochondrial integrity.

“Ligands” as used herein bind to metal via one or more atoms in the ligand, and are often termed as chelating ligands. A ligand that binds through two sites is classified as bidentate, and three sites as tridentate. The “bite angle” refers to the angle between the two bonds of a bidentate chelate. Chelating ligands are commonly formed by linking donor groups via organic linkers. A classic bidentate ligand is ethylenediamine, which is derived by the linking of two ammonia groups with an ethylene (—CH₂CH₂—) linker. A classic example of a polydentate ligand is the hexadentate chelating agent EDTA, which is able to bond through six sites, completely surrounding some metals. The binding affinity of a chelating system depends on the chelating angle or bite angle. Many ligands are capable of binding metal ions through multiple sites, usually because the ligands have lone pairs on more than one atom. Some ligands can bond to a metal center through the same atom but with a different number of lone pairs. The bond order of the metal ligand bond can be in part distinguished through the metal ligand bond angle (M-X—R). This bond angle is often referred to as being linear or bent with further discussion concerning the degree to which the angle is bent. For example, an imido ligand in the ionic form has three lone pairs. One lone pair is used as a sigma X donor, the other two lone pairs are available as L type pi donors. If both lone pairs are used in pi bonds then the M-N—R geometry is linear. However, if one or both of these lone pairs are non-bonding then the M-N—R bond is bent and the extent of the bend speaks to how much pi bonding there may be. It was found that few heteroatoms, such as nitrogen, oxygen, and sulfur atoms, interacted with zinc, ideal distances between the zinc and these heteroatoms were identified. Whereas carboxylates bound to the zinc via both monodentate and bidentate interactions, the hydroxamates bound dominantly in a bidentate manner. These results aid in the design of new inhibitors with the potential to interact with zinc in the target protein. Virtually every molecule and every ion can serve as a ligand for (or “coordinate to”) metals. Monodentate ligands include virtually all anions and all simple Lewis bases. Thus, the halides and pseudohalides are important anionic ligands whereas ammonia, carbon monoxide, and water are particularly common charge-neutral ligands. Simple organic species are also very common, be they anionic (RO⁻ and RCO₂⁻) or neutral (R₂O, R²S, R₃₋ₓNHₓ, and R₃P). Complexes of polydentate ligands are called chelate complexes. They tend to be more stable than complexes derived from monodentate ligands. This enhanced stability, the chelate effect, is usually attributed to effects of entropy, which favors the displacement of many ligands by one polydentate ligand. When the chelating ligand forms a large ring that at least partially surrounds the central atom and bonds to it, leaving the central atom at the center of a large ring. The more rigid and the higher its denticity, the more inert will be the macrocyclic complex.

“Chelator” as used herein relates to a binding agent that suppresses chemical activity by forming a chelate (a coordination compound in which a metal atom or ion is bound to a ligand at two or more points on the ligand, so as to form, for example, a heterocyclic ring containing a metal atom).

“Chelation” as used herein relates to a particular way that ions and molecules bind metal ions. According to the International Union of Pure and Applied Chemistry (IUPAC), chelation involves the formation or presence of two or more separate coordinate bonds between a polydentate (multiple bonded) ligand and a single central atom. Usually these ligands are organic compounds, and are called chelants, chelators, chelating agents, or sequestering agents.

“Electrophile” as used herein relates to species that is attracted to an electron rich center. In chemistry, an electrophile is a reagent attracted to electrons. It participates in a chemical reaction by accepting an electron pair in order to bond to a nucleophile. Because electrophiles accept electrons, they are Lewis acids. Most electrophiles are positively charged, have an atom that carries a partial positive charge, or have an atom that does not have an octet of electrons.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical, including their radioisotopes. “¹²³I” refers to the radioactive isotope of iodine having atomic mass 123. The compounds of Formula I can comprise at least one ¹²³I moiety. Throughout the present application, where structures depict a ¹²³I moiety at a certain position it is meant that the I moiety at this position is enriched for ¹²³I. In other words, the compounds contain more than the natural abundance of ¹²³I at the indicated position(s). It is not required that the compounds comprise 100% ¹²³I at the indicated positions, provided ¹²³I is present in more than the natural abundance. Typically the ¹²³I isotope is enriched to greater than 50%, greater than 60%, greater than 70%, greater than, 80% or greater than 90%, relative to ¹²⁷I. “¹⁸F” refers to the radioactive isotope of fluorine having atomic mass 18. “F” or “¹⁹F” refers to the abundant, non-radioactive fluorine isotope having atomic mass 19. The compounds of Formula I can comprise at least one ¹⁸F moiety. Throughout the present application, where structures depict a ¹⁸F moiety at a certain position it is meant that the F moiety at this position is enriched for ¹⁸F. In other words, the compounds contain more than the natural abundance of ¹⁸F at the indicated position(s). It is not required that the compounds comprise 100% ¹⁸F at the indicated positions, provided ¹⁸F is present in more than the natural abundance. Typically the ¹⁸F isotope is enriched to greater than 50%, greater than 60%, greater than 70%, greater than 80% or greater than 90%, relative to ¹⁹F.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C1-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}-R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Aralkenyl” or “arylalkenyl” refers to a radical of the formula —R_{b}-R_{c} where R_{b} is an alkenylene o group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}-R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” refers to a radical of the formula —R_{b}-R_{c} where R_{b} is an alkylene group as defined above and R_{c} is a heterocyclyl radical as defined above. Unless stated otherwise specifically in the specification, a heterocycloalkylalkyl group can be optionally substituted.

“Heterocyclylalkenyl” refers to a radical of the formula —R_{b}-R_{c} where R_{b} is an alkenylene group as defined above and R_{c} is a heterocyclyl radical as defined above. Unless stated otherwise specifically in the specification, a heterocycloalkylalkenyl group can be optionally substituted.

“Heterocyclylalkynyl” refers to a radical of the formula —R_{b}-R_{c} where R_{b} is an alkynylene group as defined above and R_{c} is a heterocyclyl radical as defined above. Unless stated otherwise specifically in the specification, a heterocycloalkylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}-R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}-R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}-R_{f} where R_{b} is an alkynylene chain as defined above and R_{f} is a heteroaryl radical as defined above. Unless stated otherwise specifically in the specification, a heteroarylalkynyl group can be optionally substituted.

“Ring” refers to a cyclic group which can be fully saturated, partially saturated, or fully unsaturated. A ring can be monocyclic, bicyclic, tricyclic, or tetracyclic. Unless stated otherwise specifically in the specification, a ring can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g} Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

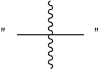

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

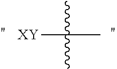

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

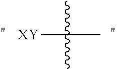

infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

Ubiquitination is crucial for a plethora of physiological processes, including cell survival and differentiation and innate and adaptive immunity. Proteins are built-up to cater for the structural and biochemical requirements of the cell and they are also broken-down in a highly-regulated process serving more purposes than just destruction and space management. Proteins have different half-lives, determined by the nature of the amino acids present at their N-termini. Some will be long-lived, while other will rapidly be degraded. Proteolysis not only enables the cell to dispose of misfolded or damaged proteins, but also to fine-tune the concentration of essential proteins within the cell, such as the proteins involved in the cell cycle. This rapid, highly specific degradation can be achieved through the addition of one to several ubiquitin molecules to a target protein. The process is called ubiquitination.

In recent years, considerable progress has been made in the understanding of the molecular action of ubiquitin in signaling pathways and how alterations in the ubiquitin system lead to the development of distinct human diseases. It has been shown that ubiquitination plays a role in the onset and progression of cancer, metabolic syndromes, neurodegenerative diseases, autoimmunity, inflammatory disorders, infection and muscle dystrophies (Popovic et al. Nature Medicine 20, 1242-1253 (2014)).

Ubiquitin-protein (E3) ligases are a large family of enzymes that select various proteins for ubiquitination. These ubiquitin ligases, called “Ub ligases” are known to have a role in various diseases and conditions, including but not limited to, cancer, inflammation and infectious diseases.

Further, there are various known methods for regulating ligases known in the art. Many ligases, particularly ligases involved in the Ubiquitin-Proteasome Pathway System (UPS), are known to have Zinc Finger (ZnF) domains that stabilize critical protein binding regions in that ligase. ZnF domains coordinate zinc ions and this coordination stabilizes functional activity of the protein. The functional activity provided by proteins with ZnF domains can include the regulation of important cellular signaling pathways, such as recognizing ubiquitins, regulation of DNA, such as transcription and repair, and acting as cellular redox sensors. The binding of zinc to ZnF domains, or simply just regulating how zinc interacts with the ZnF domains, are essential to ligases involved in the UPS.

The present invention relates to bifunctional compounds, also known as Proteolysis Targeting Chimeric ligands (Protac compounds) that induce ubiquitination by the use of a ligase, such as E3 ligase and degrade a protein of interest. Protac compounds are typically designed with three parts: 1) a ligand/molecule that binds to and/or modulates ubiquitin ligases; 2) a small molecule that binds to the target protein of interest for proteolysis; and 3) a linker that links the two molecules together. Protacs thus function by allowing the ligand/molecule to bind to the ubiquitin ligases, thereby recruiting the target of protein of interest to the ligase for ubiquitination and ultimately proteolysis and degradation. The present invention thus exhibits a broad array of applications in the pharmaceutical arts for degradation and/or inhibition of target proteins associated with disease, such as prostate cancer.

In certain embodiments, the compounds of the present invention can be used to treat diseases associated with overexpression and/or uncontrolled activation of a protein/enzyme. In a specific embodiment, the compounds are bifunctional by binding to both a ligase and a target protein of interest for inhibitition or degredation, thereby reducing and/or inhibiting the undesirable overexpression and/or uncontrolled activation of said protein target. In another specific embodiment, the compounds of the present invention include molecules that are selective in binding to a ligase, such as an E3 ligase. The present invention also also provides various options of linking the ligand/molecule that binds to and/or modulates ubiquitin ligases to the small molecule that binds to the target protein of interest. Specifically, the compounds of the present invention, are linked in such a way so that the target protein is close enough in proximity to the ligase and thus effect degradation of the target protein, such as androgen receptor proteins.

The following description includes information that may be useful in understanding the present invention. It is not an admission that any of the information provided herein is prior art or relevant to the presently claimed inventions, or that any publication specifically or implicitly referenced is prior art.

### COMPOUNDS OF THE PRESENT DISCLOSURE

The compound of the present disclosure can be useful for modifying the ubiquitination and subsequent degradation of androgen receptor proteins. In one embodiment of the present invention, the compound is a bifunctional compound wherein a ligase modulator (“PLM”), such as E3 ligase binding group, is covalently attached to one end of a Linker (“LI”), and the androgen receptor modulator (“PTC”) is covalently attached to the other end of the linker (LI). In one embodiment, androgen receptor modulator is androgen receptor N-terminal domain inhibitor. Further, the compound of the present disclosure can be useful for treating various diseases and conditions including, but not limited to, cancer.

In some embodiments, the linker is independently covalently bonded to the PLM and the PTC for example, through an amide, ester, thioester, keto, carbamate, carbon or ether, wherein the linking position can be anywhere on the PLM and/or PTC. In some embodiments, suitable linking positions provide maximum binding of the PLM to the E3 ligase and PTC to the androgen receptor protein to be degraded, as well as maximum target ubiquitination.

The linker (LI) is of a length appropriate to bring together the androgen receptor protein and E3 ligase and thereby elicit the ubiquitination of the protein of interest and it's subsequent degradation in the proteasome. It is therefore understood that the LI of the present disclosure serves as a spacer, physically separating the PLM and the PTC to a degree sufficient to ensure that binding with their respective targets occurs. In some embodiments, the length of the linker is optimized to maximize binding affinity between the PTC and androgen receptor protein, and the PLM and E3 ligase, as well as maximize target ubiquitination.

In one embodiment of the present disclosure, a compound of the invention comprises a ligase modulater moiety, a linker moiety, and a protein target compound moiety.

In one embodiment of the present disclosure, a compound of the invention has the structure of formula (Q):

PLM-LI-PTC (Q);

or a pharmaceutically acceptable salt thereof, wherein:

PLM is a ligase modulator, such as a parkin ligase modulator

LI is a linker, and

PTC is a protein target compound, i.e., a molecule that binds to and/or inhibits/activates a protein target of interest.

In some embodiments, the dash “-” indicated between PLM and LI or LI and PTC in formula (Q) represents each component's spacial orientation and not strictly as a C—C bond. In one embodiment, the PLM can be discussed as its own component having a chemical group necessary to covalently attach to LI. In one embodiment, the PTC can be discussed as its own component having a chemical group necessary to covalently attach to LI. One skilled in the art would readily understand how each component, described separately, can covalently attach to one another to provide a compound of formula (Q).

In one embodiment, the compound of the present disclosure is represented by formula (Q):

PLM-LI-PTC (Q);

or a pharmaceutically acceptable salt thereof, wherein:

PLM is a E3 ligase binding group,

LI is a linker, and

PTC is an androgen receptor modulator represented by formula (IIIA):

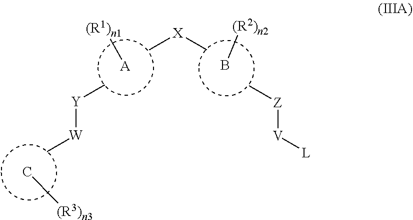

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷;

Y is a bond, —(CR⁸R⁹)ₘ—, —O—, —S—, —S(═O)—, —SO₂—, —NR⁷—, or —N(COCH₃)—;

W is a bond, —(CR^{8a}R^{9a})ₘ—, —C(═O)—, —N(R⁷)CO—, —CONR⁷—, or —NSO₂R⁷—;

Z is a bond, —CH₂—, —C(CH₃)H—, —O—, —S—, —NH—, —NCH₃—, or —N(COCH₃)—;

V is —CH₂— and L is halogen, —NH₂, —CHCl₂, —CCl₃, or —CF₃; or

V is —CH₂CH₂— and L is halogen or —NH₂;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is hydrogen, C₁-C₆ alkyl, C₂-C₆ alkenyl, or C₂-C₆ alkynyl;

R⁸ and R⁹ are each independently hydrogen, halogen, or C₁-C₃ alkyl;

R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;

R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;

each m is independently 0, 1, or 2;

n1 and n2 are each independently 0, 1, or 2;

n3 is 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, PTC in formula Q is a compound of formula (IIIA) minus any functional group that was involved in making the PTC-LI bond.

In some embodiments of the present disclosure, PLM is an E3 ligase modulator.

In some embodiments, the dash “-” indicated between PLM and LI or LI and PTC in formula (Q) represents each component's spacial orientation and not strictly as a C—C bond. In one embodiment, the PLM can be discussed as its own component having a chemical group necessary to covalently attach to LI. In one embodiment, the PTC can be discussed as its own component having a chemical group necessary to covalently attach to LI. One skilled in the art would readily understand how each component, described separately, can covalently attach to one another to provide a compound of formula (Q).

In some embodiments, the compound of formula (Q) is a compound of formula (W-IV), (W-IVA), (W-V), (W-VA), (W-VI), (W-VIA), (VII), (VIII), (IX) or (X):

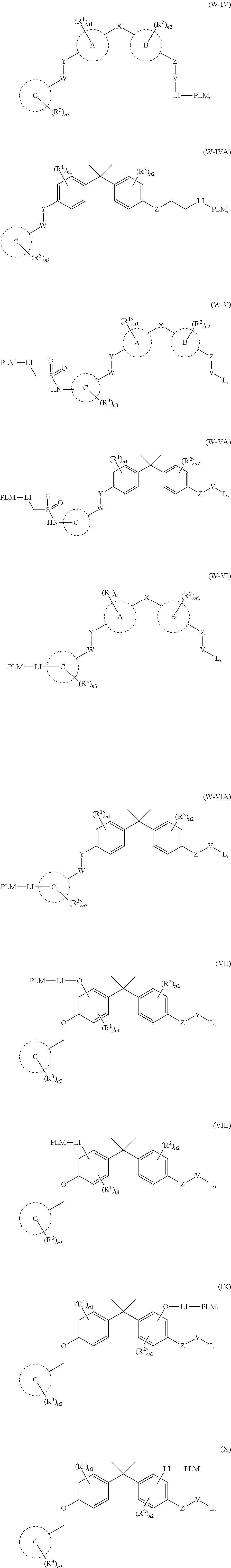

or a pharmaceutically acceptable salt thereof, wherein A, B, C, R¹, R², R³, Z, V, L, Y, W, LI, PLM, n1, n2, and n3 are as defined herein.

In some embodiments the compound is selected from Table P:

TABLE P

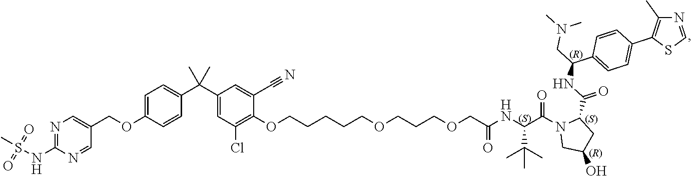

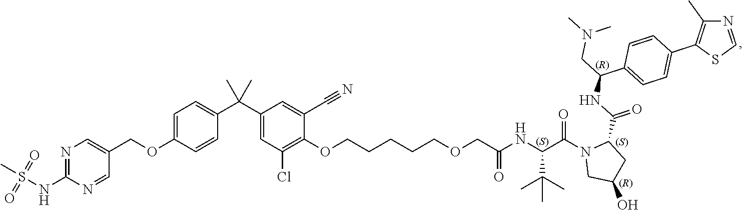

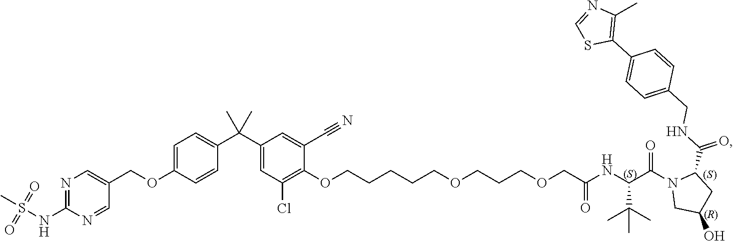

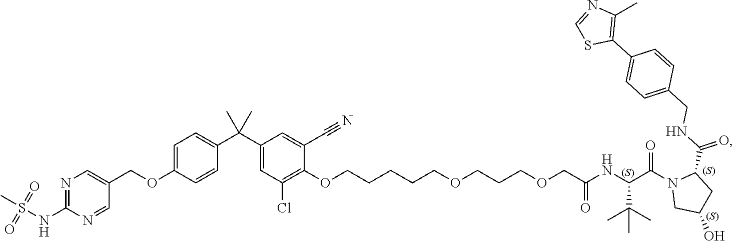

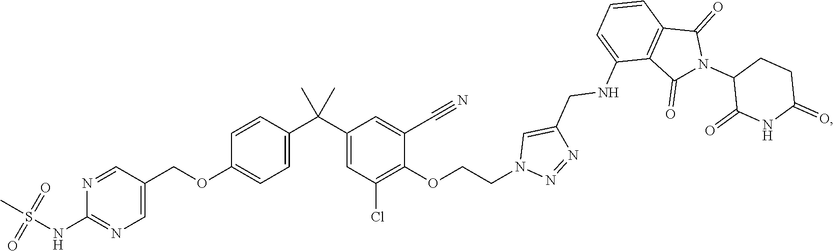 [SMILES: CC(C)(c1ccc(OCc2cnc(NS(C)(=O)=O)nc2)cc1)c1cc(Cl)c(OCCn2cc(CNc3cccc4c3C(=O)N(C3CCC(=O)NC3=O)C4=O)nn2)c(C#N)c1]

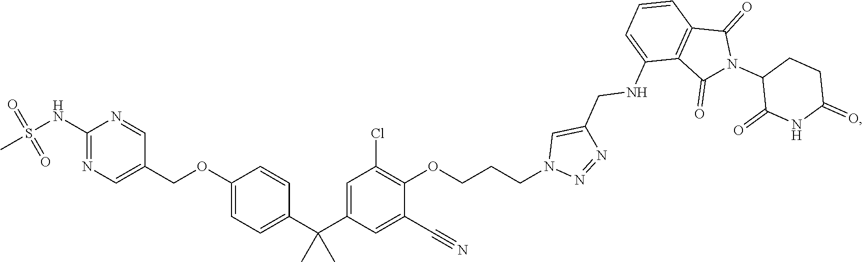 [SMILES: CC(C)(c1ccc(OCc2cnc(NS(C)(=O)=O)nc2)cc1)c1cc(Cl)c(OCCCn2cc(CNc3cccc4c3C(=O)N(C3CCC(=O)NC3=O)C4=O)nn2)c(C#N)c1]

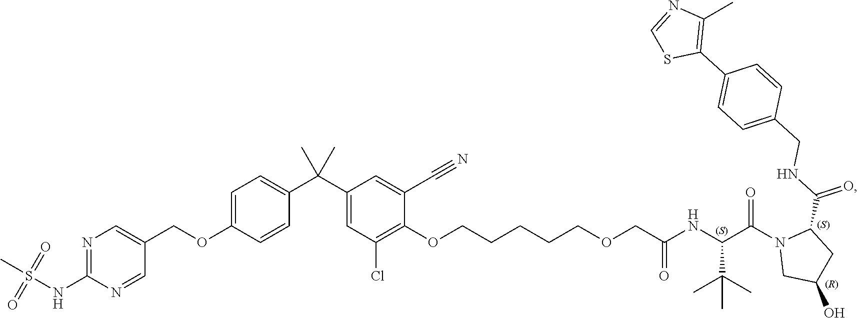

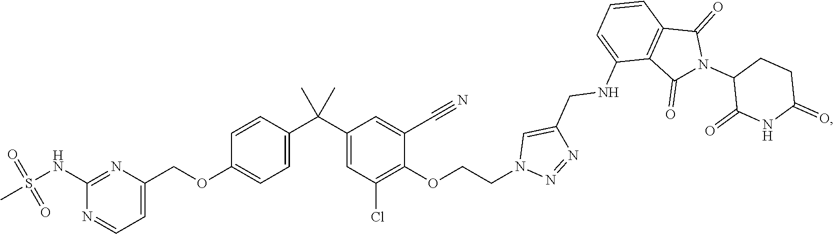 [SMILES: CC(C)(c1ccc(OCc2ccnc(NS(C)(=O)=O)n2)cc1)c1cc(Cl)c(OCCn2cc(CNc3cccc4c3C(=O)N(C3CCC(=O)NC3=O)C4=O)nn2)c(C#N)c1]

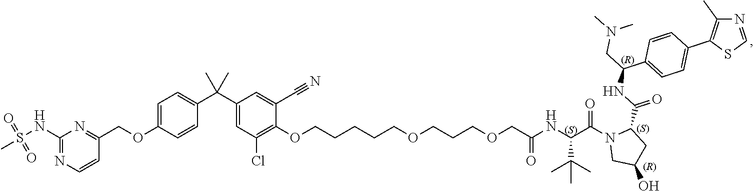

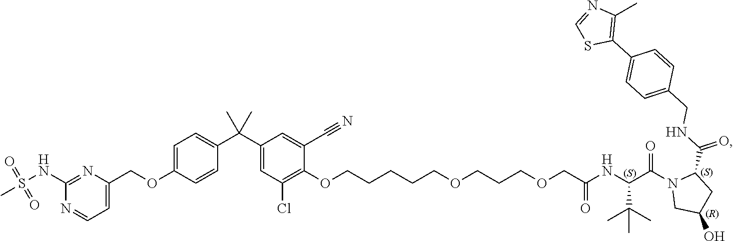

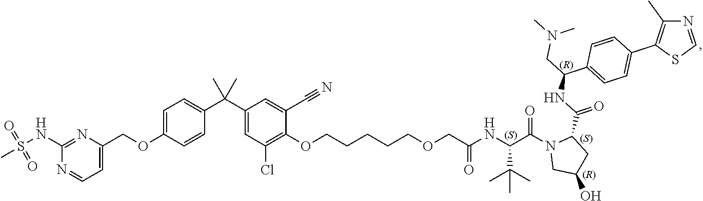

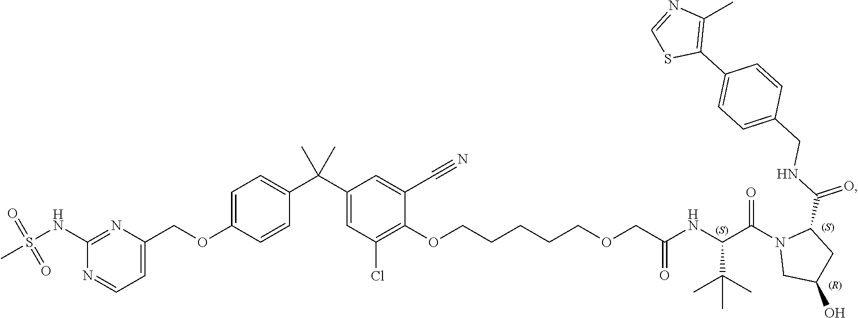

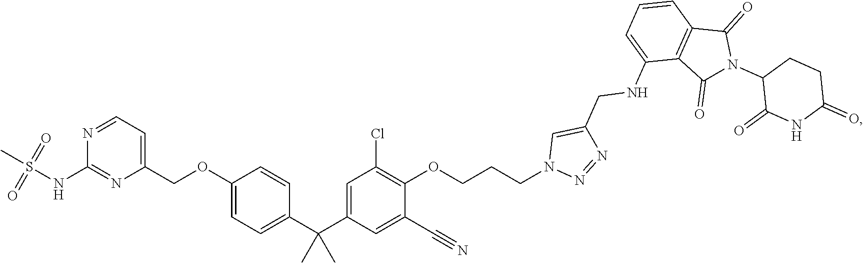 [SMILES: CC(C)(c1ccc(OCc2ccnc(NS(C)(=O)=O)n2)cc1)c1cc(Cl)c(OCCCn2cc(CNc3cccc4c3C(=O)N(C3CCC(=O)NC3=O)C4=O)nn2)c(C#N)c1]

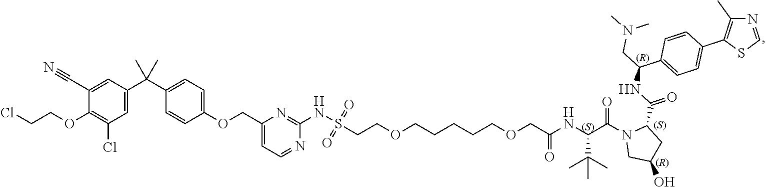

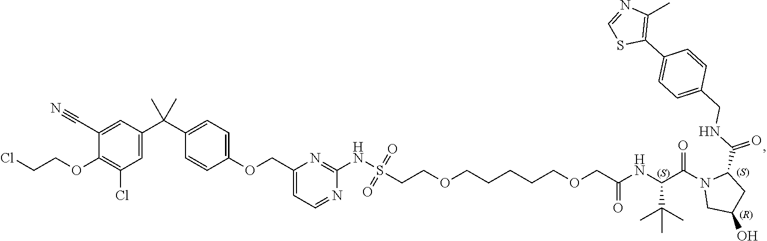

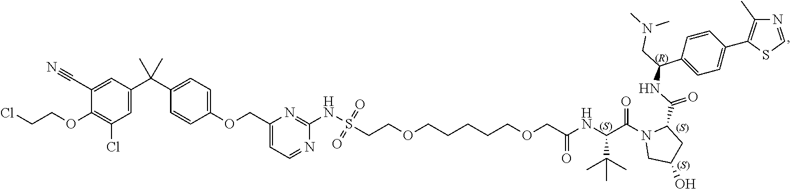

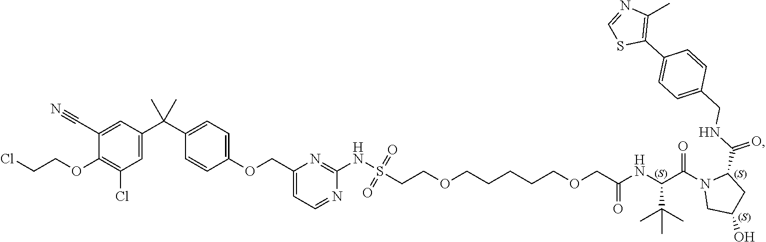

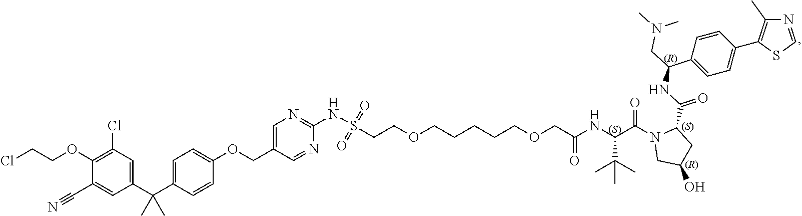

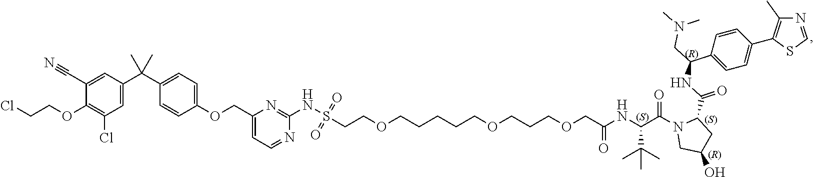

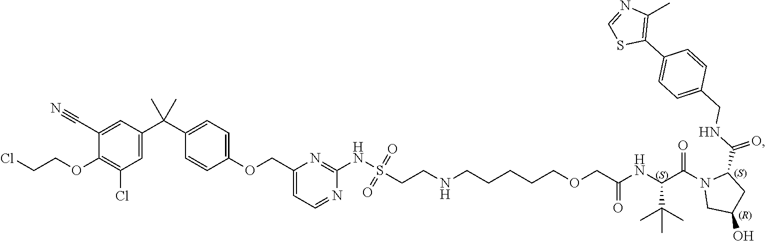

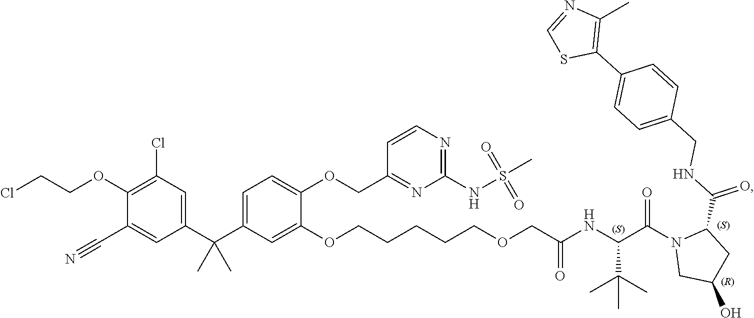

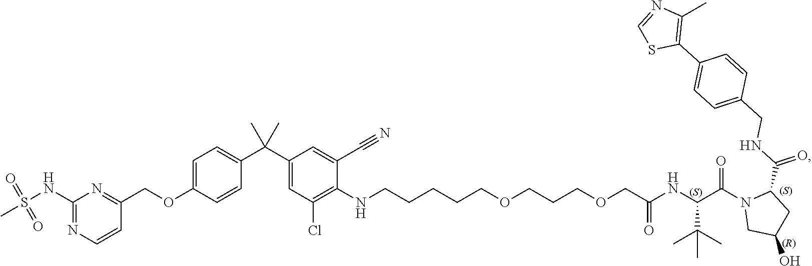

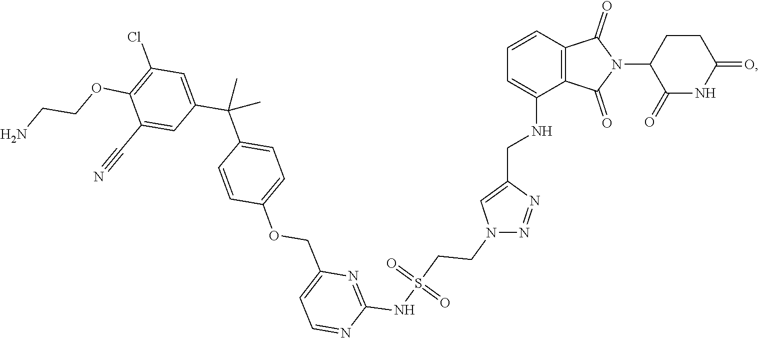 [SMILES: CC(C)(c1ccc(OCc2ccnc(NS(=O)(=O)CCn3cc(CNc4cccc5c4C(=O)N(C4CCC(=O)NC4=O)C5=O)nn3)n2)cc1)c1cc(Cl)c(OCCN)c(C#N)c1]

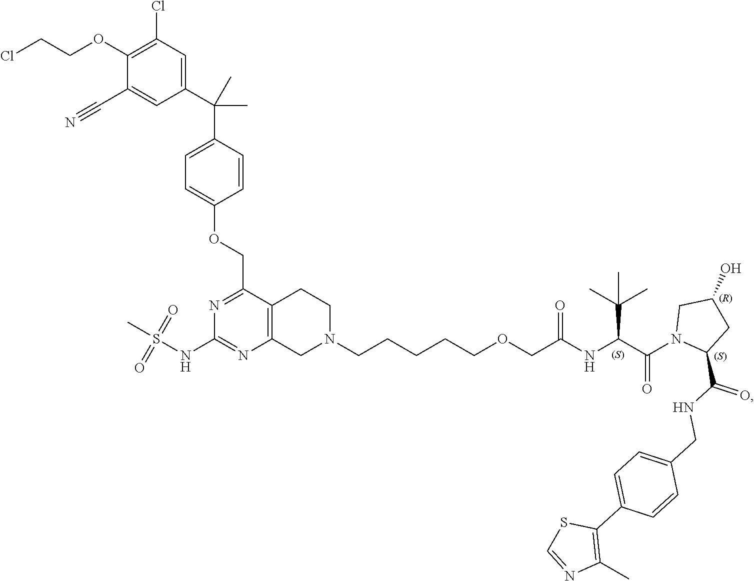

or a pharmacetucially acceptable salt thereof.

Linkers (LI)

In one embodiment, any of the LI disclosed herein can be the linker as covalently attached to the PLM and/or to the PTC. In certain embodiments, any of the LI disclosed herein can describe the linker moiety before covalently attaching it to the PLM and/or to the PTC. In anon limited example, LI can comprise a chemical group (e.g., alcohol, amine, azides, —C≡CH, etc) which can be reacted with another chemical group on or attached to the PLM or the PTC in order to form a covalent bond, e.g., amine bond, ether bond, amide bond, ester bond, triazole (Click chemistry). In one embodiment, a chemical group already present in the LI as described herein can be used to covalently attach the LI to the PLM and/or to the PTC. The chemistry used to covalently attach the PLM to the LI and LI to the PTC can be readily understood by one skilled in the art.

In one embodiment, any of the LI disclosed herein can further comprise a chemical group useful in covalently attaching LI to the PLM and/or to the PTC.

In some embodiments of the compound of formula (Q), the linker LI corresponds to formula

-LX_{A}-(CH₂)ₘ₁—(CH₂—CH₂-LX_{B})ₘ₂—(CH₂)ₘ₃-LX_{C}-, wherein:

-LX_{A} is covalently bound to the PTC or PLM, and LX_{C}- is covalently bound to the PLM or PTC;

each m1 and m2 is independently 0, 1, 2, 3, 4, 5, 6, 7, 8, 9, 10, 11, or 12;

m3 is independently 1, 2, 3, 4, 5, 6, 7, 8, 9, 10, 11, or 12;

LX_{A} is absent (a bond), —CH₂C(O)NR²⁰—, or —NR²⁰C(O)CH₂—;

LX_{B} and LX_{C} are each independently absent (a bond), —CH₂—, —O—, —S—, —S(O)—, —S(O)₂, or —N(R²⁰)—;

wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and

wherein each —CH₂— in the linker is optionally substituted.

In some embodiments of the compound of formula (Q), LX_{A} is absent (a bond), —CH₂C(O)NR²⁰—, or —NR²⁰C(O)CH₂—; wherein R²⁰ is hydrogen or C₁-C₃ alkyl.

In some embodiments of the compound of formula (Q), LX_{A} is absent (a bond), —CH₂C(O)NR²⁰—, or —NR²⁰C(O)CH₂—; wherein R²⁰ is hydrogen, deuterium, halogen, or C₁-C₃ alkyl.

In some embodiments of the compound of formula (Q), LX_{A} is absent (a bond), —CH₂C(O)NH—, —NHC(O)CH₂—.

In one embodiment, LX_{B} is absent (a bond), —CH₂—, —O—, or —N(R²⁰)—; wherein R²⁰ is hydrogen, deuterium, halogen, or C₁-C₃ alkyl.

In some embodiments of the compound of formula (Q), LX_{B} is absent (a bond), —CH₂—, —O— or —N(R²⁰)—; wherein R²⁰ is hydrogen or C₁-C₃ alkyl.

In some embodiments of the compound of formula (Q), LX_{C} is absent (a bond), —CH₂—, —O—, or —NH—.

In some embodiments of the compound of formula (Q), m1 is 0, 1, 2, 3, 4, 5, 6, 7, 8, 9, or 10. In one embodiment, m2 is 1, 2, 3, 4, 5, 6, 7, 8, 9, or 10. In one embodiment, m3 is 1, 2, 3, 4, 5, or 6.

In one embodiment, the sum of m1, m2, and m3 is less than or equal to 24. In one embodiment, the sum of m1, m2, and m3 is less than or equal to 24, less than or equal to 23, less than or equal to 22, less than or equal to 21, less than or equal to 20, less than or equal to 19, less than or equal to 18, less than or equal to 17, less than or equal to 16, less than or equal to 15, less than or equal to 14, less than or equal to 13, or less than or equal to 12.

In one embodiment, the sum of m1, m2, and m3 is less than or equal to 12. In one embodiment, the sum of m1, m2, and m3 is less than or equal to 13. In one embodiment, the sum of m1, m2, and m3 is less than or equal to 12. In one embodiment, the sum of m1, m2, and m3 is less than or equal to 11. In one embodiment, the sum of m1, m2, and m3 is less than or equal to 10. In one embodiment, the sum of m1, m2, and m3 is less than or equal to 9. In one embodiment, the sum of m1, m2, and m3 is less than or equal to 8. In one embodiment, the sum of m1, m2, and m3 is less than or equal to 7. In one embodiment, the sum of m1, m2, and m3 is less than or equal to 6. In one embodiment, the sum of m1, m2, and m3 is less than or equal to 5.

In one embodiment, the total number of atoms in a straight chain between PTC and PLM is 20 or less.

In some embodiments of the compound of formula (Q), the linker LI corresponds to formula:

—(CH₂—CH₂—O)ₘ₂—CH₂CH₂-LX_{C}-;

—CH₂C(O)NH—(CH₂—CH₂)ₘ₂—CH₂CH₂-LX_{C}-;

—CH₂C(O)NH—(CH₂—CH₂—O)ₘ₂—CH₂-LX_{C}-;

—CH₂C(O)NH—(CH₂—CH₂—O)ₘ₂—CH₂CH₂-LX_{C}-; or

—CH₂C(O)NH—CH₂—(CH₂—CH₂—O)ₘ₂—CH₂CH₂CH₂-LX_{C}-; wherein —(CH₂—CH₂—O)ₘ₂ or —CH₂C(O)NH or is covalently bound to the PTC or PLM, and LX_{C}- is covalently bound to the PLM or PTC;

m2 is independently 1, 2, 3, 4, 5, or 6;

LX_{C} are each independently absent (a bond), —CH₂—, —O—, —S—, —S(O)—, —S(O)₂—, or —N(R²⁰)—;

wherein each R²⁰ is hydrogen or C₁-C₃ alkyl; and

wherein each —CH₂— in the linker is optionally substituted.

In some embodiments of the compound of formula (Q), the linker LI corresponds to formula

—(CH₂)ₘ₁-LX₁-(CH₂—CH₂-LX₂)ₘ₂—(CH₂)ₘ₃—C(LX₃)-, wherein:

—(CH₂)ₘ₁ is covalently bound to the PTC or PLM, and C(LX₃)- is covalently bound to the PLM or PTC;

each m1, m2, and m3 is independently 0, 1, 2, 3, 4, 5, 6, 7, 8, 9, or 10; and

each LX₁, LX₂, and LX₃ is independently absent (a bond), —O—, —S—, —S(O)—, —S(O)₂—, or —N(R²⁰)—, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and

wherein each —CH₂— in the linker is optionally substituted. In one embodiment, the sum of m1, m2, and m3 is less than or equal to 24. In one embodiment, the sum of m1, m2, and m3 is less than or equal to 24, less than or equal to 23, less than or equal to 22, less than or equal to 21, less than or equal to 20, less than or equal to 19, less than or equal to 18, less than or equal to 17, less than or equal to 16, less than or equal to 15, less than or equal to 14, less than or equal to 13, or less than or equal to 12.

In some embodiments of the compound of formula (Q), LX₁, LX₂, and LX₃ are —O—.

In some embodiments of the compound of formula (Q), the Linker corresponds to formula

—(CH₂)ₘ₁-LX_{B}-(CH₂)ₘ₂-LX_{C}-(CH₂)ₘ₃-LX_{D}-(CH₂)ₘ₄—C(O)—, wherein:

(CH₂)ₘ₁ is covalently bound to the PTC or PLM, and C(O) is covalently bound to the PLM or PTC;

each m1, and m2 is independently 0, 1, 2, 3, 4, 5, 6, 7, 8, 9, 10, 11, or 12;

m3 is independently 1, 2, 3, 4, 5, 6, 7, 8, 9, 10, 11, or 12;

m4 is 0, 1, 2, 3, 4, 5, 6, 7, 8, 9, 10, 11, or 12;

LX_{B}, LX_{C}, and LX_{D} are each independently absent (a bond), —CH₂—, —O—, —S—, —S(O)—, —S(O)₂, or —N(R²⁰)—;

wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and

wherein each —CH₂— in the linker is optionally substituted. In one embodiment, the sum of m1, m2, m3 and m4 is less than or equal to 24. In one embodiment, the sum of m1, m2, m3, and m4 is less than or equal to 23, less than or equal to 22, less than or equal to 21, less than or equal to 20, less than or equal to 19, less than or equal to 18, less than or equal to 17, less than or equal to 16, less than or equal to 15, less than or equal to 14, less than or equal to 13, or less than or equal to 12.

In some embodiments of the compound of formula (Q), the Linker corresponds to formula

—(CH₂)ₘ₁-LX_{B}-(CH₂)ₘ₂-LX_{C}-(CH₂)ₘ₃—O—(CH₂)ₘ₄—C(O)—, wherein:

(CH₂)ₘ₁ is covalently bound to the PTC, and C(O) is covalently bound to the PLM;

m1 is 0, 1, 2, or 3;

m2 is independently 0, 1, 2, 3, 4, or 5;

m3 is independently 1, 2, 3, 4, or 5;

m4 is 1, 2 or 3;

LX_{B} and LX_{C} are each independently absent (a bond), —O— or —N(R²⁰)—;

wherein each R²⁰ is independently selected from the group consisting of hydrogen, deuterium, and C₁-C₆ alkyl.

In some embodiments of the compound of formula (Q), the linker LI is a polyethylene glycol chain ranging in size from about 1 to about 12 ethylene glycol units, wherein each —CH₂— in the polyethylene glycol is optionally substituted. In some embodiments, the linker LI is a polyethylene glycol chain ranging in size from about 2 to about 10 ethylene glycol units, wherein each —CH₂— in the polyethylene glycol is optionally substituted. In some embodiments, the linker LI is a polyethylene glycol chain ranging in size from about 3 to about 5 ethylene glycol units, wherein each —CH₂— in the polyethylene glycol is optionally substituted.

In some embodiments of the compound of formula (Q), the linker LI corresponds to the formula:

-L_{I}-L_{II}(q)-,

wherein:

L_{I} is a bond or a chemical group coupled to at least one of a PLM, a PTC or a combination thereof,

L_{II} is a bond or a chemical group coupled to at least one of a PLM, a PTC, and q is an integer greater than or equal to 0;

wherein each L_{I} and L_{II} is independently selected from a bond, CR^{L1}R^{L2}, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, NR^{L3} SO₂NR^{L3}, SONR^{L3}CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁ cycloalkyl optionally substituted with 0-6 R^{L1} and/or R^{L2} groups, C₃₋₁₁ heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups;

wherein i is 1, 2, 3, 4, 5, 6, 7, 8, 9 or 10; and

wherein R^{L1}, R^{L2}, R^{L3}, R^{L4} and R^{L5} are, each independently, H, halo, —C₁₋₈ alkyl, —OC₁₋₈ alkyl, —SC₁₋₈ alkyl, —NHC₁₋₈ alkyl, —N(C₁₋₈ alkyl)₂, —C₃₋₁₁ cycloalkyl, aryl, heteroaryl, —C₃₋₁₁ heterocyclyl, —OC₁₋₈ cycloalkyl, —SC₁₋₈ cycloalkyl, —NHC₁₋₈ cycloalkyl, —N(C₁₋₈ cycloalkyl)₂, —N(C₁₋₈ cycloalkyl)(C₁₋₈ alkyl), —OH, —NH₂, —SH, —SO₂C₁₋₈ alkyl, —P(O)(OC₁₋₈ alkyl)(C₁₋₈ alkyl), —P(O)(OC₁₋₈ alkyl)₂, —C≡C—C₁₋₈ alkyl, —CCH, —CH═CH(C₁₋₈ alkyl), —C(C₁₋₈ alkyl)=CH(C₁₋₈ alkyl), —C(C₁₋₈ alkyl)=C(C₁₋₈ alkyl)₂, —Si(OH)₃, —Si(C₁₋₈ alkyl)₃, —Si(OH)(C₁₋₈ alkyl)₂, —C(═O)C₁₋₈ alkyl, —CO₂H, halogen, —CN, —CF₃, —CHF₂, —CH₂F, —NO₂, —SF₅, —SO₂NHC₁₋₈ alkyl, —SO₂N(C₁₋₈ alkyl)₂, —SONHC₁₋₈ alkyl, —SON(C₁₋₈ alkyl)₂, —CONHC₁₋₈ alkyl, —CON(C₁₋₈ alkyl)₂, —N(C₁₋₈ alkyl)CONH(C₁₋₈ alkyl), —N(C₁₋₈ alkyl)CON(C₁₋₈ alkyl)₂, —NHCONH(C₁₋₈ alkyl), —NHCON(C₁₋₈ alkyl)₂, —NHCONH₂, —N(C₁₋₈ alkyl)SO₂NH(C₁₋₈ alkyl), —N(C₁₋₈ alkyl)SO₂N(C₁₋₈ alkyl)₂, —NHSO₂NH(C₁₋₈ alkyl), —NHSO₂N(C₁₋₈ alkyl)₂, or —NHSO₂NH₂.

In some embodiments of the compound of formula (Q), q is selected from 1, 2, 3, 4, 5, 6, 7, 8, 9, 10, 11, 12, 13, 14, 15, 16, 17, 18, 19, 20, 21, 22, 23, or 24.

In some embodiments of the compound of formula (Q), L_{I} and L_{II} are independently selected from a bond, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, wherein i is 1, 2, 3, 4, 5, 6, 7, 8, 9 or 10, and at least one of L_{I} and L_{II} is not a bond.

In some embodiments of the compound of formula (Q), the linker LI is selected from Table L1, wherein LI is covalently bound to PLM by replacing a hydrogen from LI with a covalent bond to the PLM; and wherein LI is covalently bound to PTC by replacing a hydrogen from LI with a covalent bond the PTC.

TABLE L1
2-(3-(5-(tosyloxy)pentyloxy)propoxy)acetic acid;
2-(3-(3,3-dimethyl-5-(tosyloxy)pentyloxy)propoxy)acetic acid;
2-(3-(3-hydroxy-5-(tosyloxy)pentyloxy)propoxy)acetic acid;
2-(2-(2-(2-(tosyloxy)ethoxy)ethoxy)ethoxy)acetic acid;
2-(2-((2R,3R)-3-(2-(tosyloxy)ethoxy)butan-2-yloxy)ethoxy)acetic acid;
2-(2-((2S,3S)-3-(2-(tosyloxy)ethoxy)butan-2-yloxy)ethoxy)acetic acid;
2-(4-(4-(tosyloxy)butoxy)butoxy)acetic acid;
tert-butyl 2-(3-(4-(tosyloxy)butoxy)propoxy)acetate;
tert-butyl 2-(4-(3-(tosyloxy)propoxy)butoxy)acetate;
tert-butyl 2-(6-(tosyloxy)hexa-2,4-diynyloxy)acetate;
tert-butyl 3-(6-(tosyloxy)hexa-2,4-diynyloxy)propanoate;
tert-butyl 4-(6-(tosyloxy)hexa-2,4-diynyloxy)butanoate;
ethyl 2-(2-(2-aminoethoxy)ethoxy)acetate hydrochloride;
ethyl 2-(5-aminopentyloxy)acetate;
methyl 2-(2-(2-(methylamino)ethoxy)ethoxy)acetate;
ethyl 2-(5-(methylamino)pentyloxy)acetate;
2-(3-(2-(tosyloxy)ethoxy)propoxy)acetic acid;
2-(2-hydroxyethoxy)ethyl 4-methylbenzenesulfonate;
ethyl 2-(2-(2-(tosyloxy)ethoxy)ethoxy)acetate;
ethyl 3-(2-(2-(tosyloxy)ethoxy)ethoxy)propanoate;
ethyl 5-(tosyloxy)pentanoate;
ethyl 3-(2-(tosyloxy)ethoxy)propanoate;
ethyl 2-(5-(tosyloxy)pentyloxy)acetate;
ethyl 3-(5-(tosyloxy)pentyloxy)propanoate;
5-hydroxypentyl 4-methylbenzenesulfonate;
ethyl 2-(5-(tosyloxy)pentyloxy)acetate;
ethyl 2-(3-(tosyloxy)propoxy)acetate;
ethyl 2-(2-(tosyloxy)ethoxy)acetate;
ethyl 2-(4-(2-(tosyloxy)ethoxy)butoxy)acetate;
2-(2-(2-hydroxyethoxy)ethoxy)ethyl 4-methylbenzenesulfonate;
2-((2R,3R)-3-(2-hydroxyethoxy)butan-2-yloxy)ethyl
4-methylbenzenesulfonate;
2-(2-piperazin-1-yl)-ethoxy-acetic acid; and
methyl 6-(4-(2-(2-(tert-butoxy)-2-oxoethoxy)ethyl)piperazin-1-
yl)nicotinate.

In some embodiments of the compound of formula (Q), the linker LI is selected from Table L2:

TABLE L2

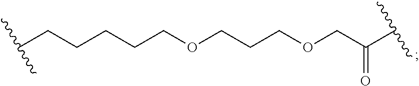 [SMILES: *CCCCCOCCCOCC(*)=O]

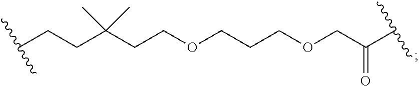 [SMILES: *CCC(C)(C)CCOCCCOCC(*)=O]

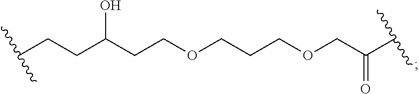 [SMILES: *CCC(O)CCOCCCOCC(*)=O]

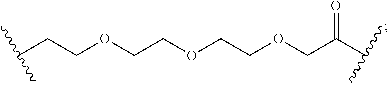 [SMILES: *CCOCCOCCOCC(*)=O]

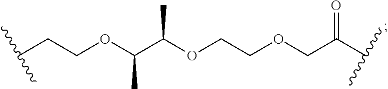 [SMILES: *CCO[C@H](C)[C@@H](C)OCCOCC(*)=O]

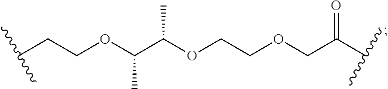 [SMILES: *CCO[C@@H](C)[C@H](C)OCCOCC(*)=O]

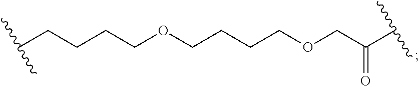 [SMILES: *CCCCOCCCCOCC(*)=O]

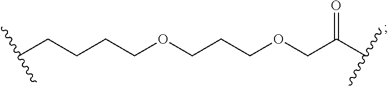 [SMILES: *CCCCOCCCOCC(*)=O]

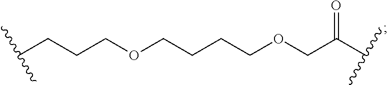 [SMILES: *CCCOCCCCOCC(*)=O]

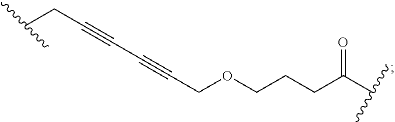 [SMILES: *CC#CC#CCOCCCC(*)=O]

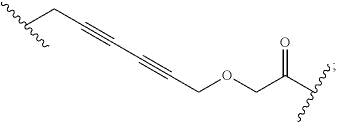 [SMILES: *CC#CC#CCOCC(*)=O]

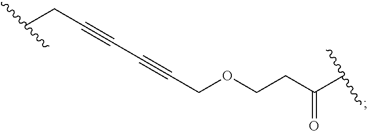 [SMILES: *CC#CC#CCOCCC(*)=O]

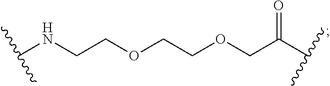 [SMILES: *NCCOCCOCC(*)=O]

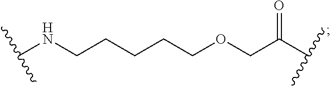 [SMILES: *NCCCCCOCC(*)=O]

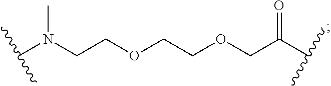 [SMILES: *C(=O)COCCOCCN(*)C]

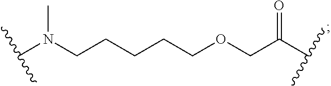 [SMILES: *C(=O)COCCCCCN(*)C]

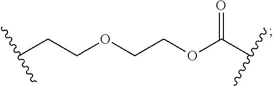 [SMILES: *CCOCCOC(*)=O]

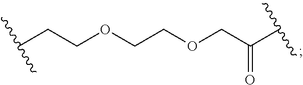 [SMILES: *CCOCCOCC(*)=O]

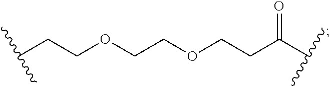 [SMILES: *CCOCCOCCC(*)=O]

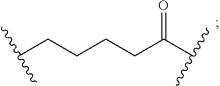 [SMILES: *CCCCC(*)=O]

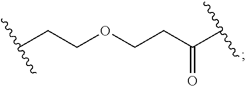 [SMILES: *CCOCCC(*)=O]

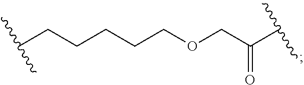 [SMILES: *CCCCCOCC(*)=O]

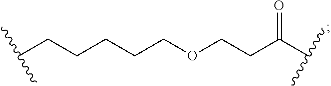 [SMILES: *CCCCCOCCC(*)=O]

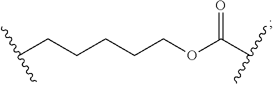 [SMILES: *CCCCCOC(*)=O]

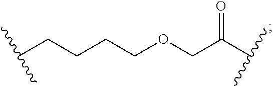 [SMILES: *CCCCOCC(*)=O]

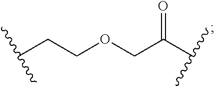 [SMILES: *CCOCC(*)=O]

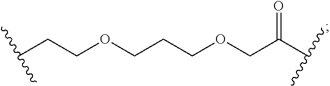 [SMILES: *CCOCCCOCC(*)=O]

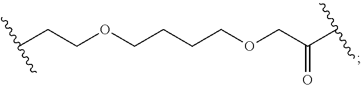 [SMILES: *CCOCCCCOCC(*)=O]

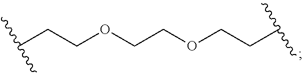 [SMILES: *CCOCCOCC*]

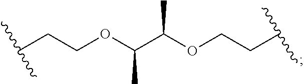 [SMILES: *CCO[C@H](C)[C@@H](C)OCC*]

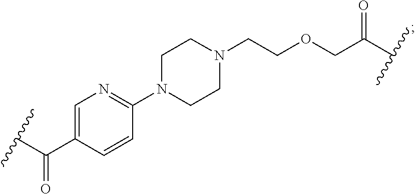 [SMILES: *C(=O)COCCN1CCN(c2ccc(C(*)=O)cn2)CC1]

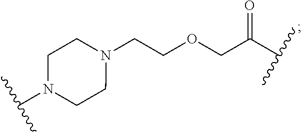 [SMILES: *C(=O)COCCN1CCN(*)CC1]

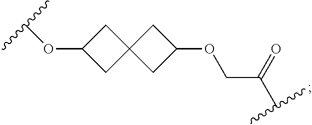 [SMILES: *OC1CC2(C1)CC(OCC(*)=O)C2]

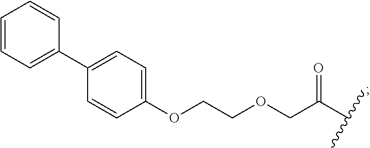 [SMILES: *C(=O)COCCOc1ccc(-c2ccccc2)cc1]

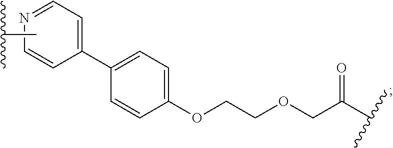

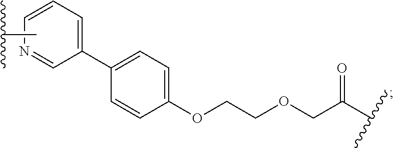

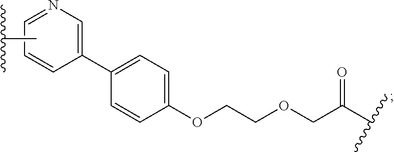

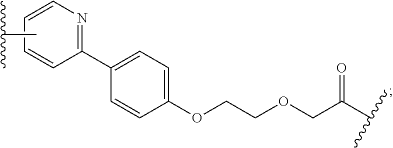

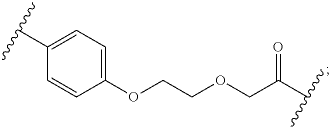 [SMILES: *C(=O)COCCOc1ccc(*)cc1]

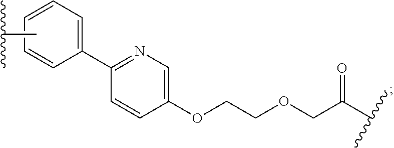

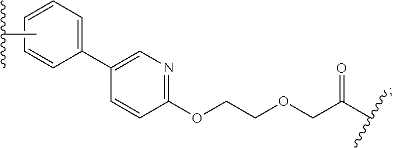

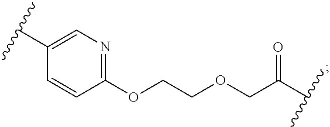 [SMILES: *C(=O)COCCOc1ccc(*)cn1]

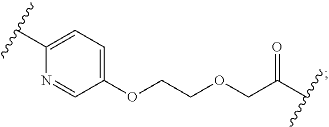 [SMILES: *C(=O)COCCOc1ccc(*)nc1]

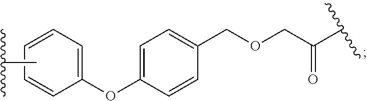

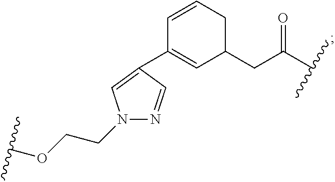 [SMILES: *OCCn1cc(C2=CC(CC(*)=O)CC=C2)cn1]

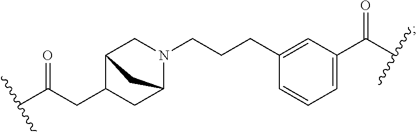 [SMILES: *C(=O)CC1C[C@@H]2C[C@H]1CN2CCCc1cccc(C(*)=O)c1]

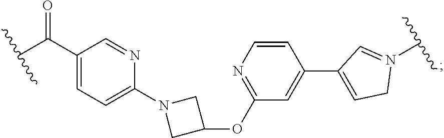

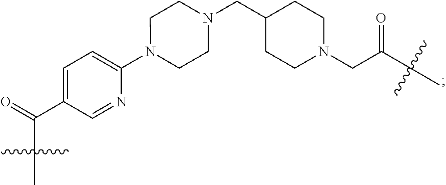 [SMILES: *C(=O)CN1CCC(CN2CCN(c3ccc(C(*)=O)cn3)CC2)CC1]

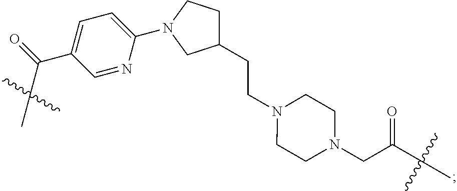 [SMILES: *C(=O)CN1CCN(CCC2CCN(c3ccc(C(*)=O)cn3)C2)CC1]

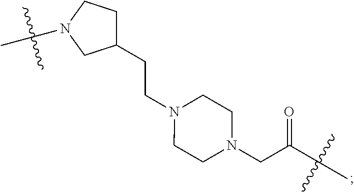 [SMILES: *C(=O)CN1CCN(CCC2CCN(*)C2)CC1]

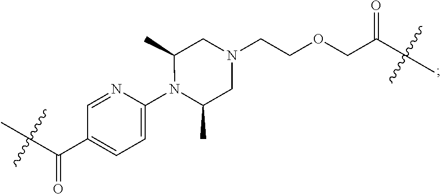 [SMILES: *C(=O)COCCN1C[C@@H](C)N(c2ccc(C(*)=O)cn2)[C@@H](C)C1]

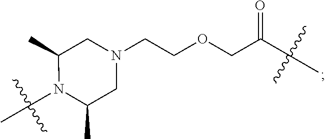 [SMILES: *C(=O)COCCN1C[C@@H](C)N(*)[C@@H](C)C1]

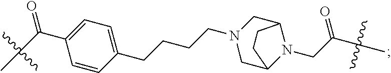 [SMILES: *C(=O)CN1C2CCC1CN(CCCCc1ccc(C(*)=O)cc1)C2]

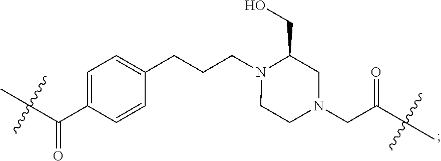 [SMILES: *C(=O)CN1CCN(CCCc2ccc(C(*)=O)cc2)[C@@H](CO)C1]

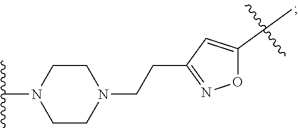 [SMILES: *c1cc(CCN2CCN(*)CC2)no1]

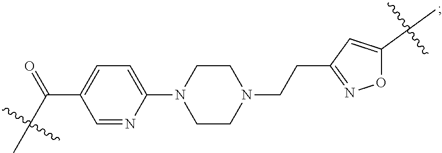 [SMILES: *C(=O)c1ccc(N2CCN(CCc3cc(*)on3)CC2)nc1]

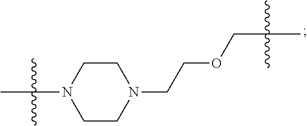 [SMILES: *COCCN1CCN(*)CC1]

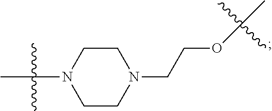 [SMILES: *OCCN1CCN(*)CC1]

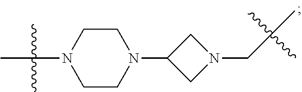 [SMILES: *CN1CC(N2CCN(*)CC2)C1]

or

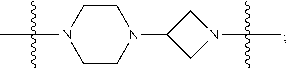 [SMILES: *N1CCN(C2CN(*)C2)CC1]

In some embodiments of the compound of formula (Q), the linker LI is selected from Table L3:

TABLE L3

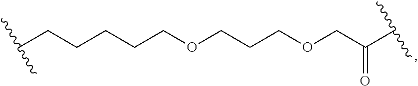 [SMILES: *CCCCCOCCCOCC(*)=O]

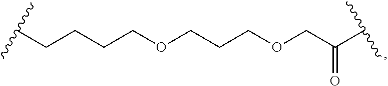 [SMILES: *CCCCOCCCOCC(*)=O]

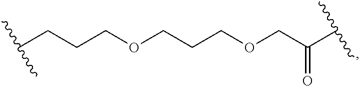 [SMILES: *CCCOCCCOCC(*)=O]

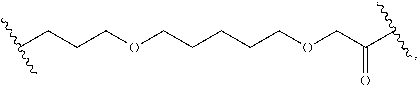 [SMILES: *CCCOCCCCCOCC(*)=O]

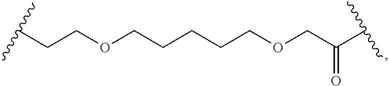 [SMILES: *CCOCCCCCOCC(*)=O]

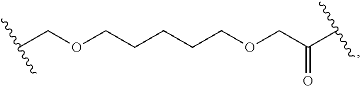 [SMILES: *COCCCCCOCC(*)=O]

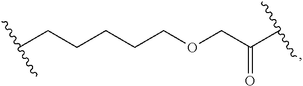 [SMILES: *CCCCCOCC(*)=O]

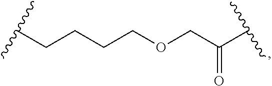 [SMILES: *CCCCOCC(*)=O]

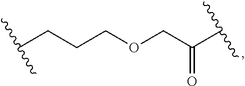 [SMILES: *CCCOCC(*)=O]

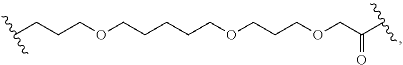 [SMILES: *CCCOCCCCCOCCCOCC(*)=O]

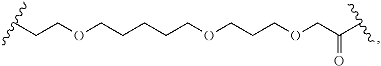 [SMILES: *CCOCCCCCOCCCOCC(*)=O]

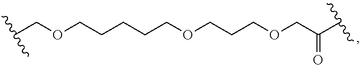 [SMILES: *COCCCCCOCCCOCC(*)=O]

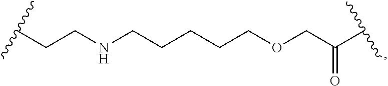 [SMILES: *CCNCCCCCOCC(*)=O]

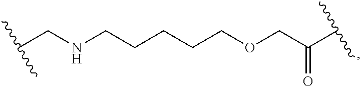 [SMILES: *CNCCCCCOCC(*)=O]

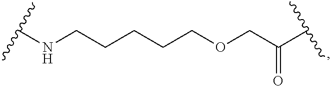 [SMILES: *NCCCCCOCC(*)=O]

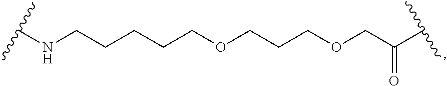 [SMILES: *NCCCCCOCCCOCC(*)=O]

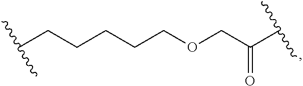 [SMILES: *CCCCCOCC(*)=O]

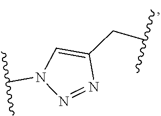 [SMILES: *Cc1cn(*)nn1]

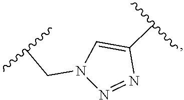 [SMILES: *Cn1cc(*)nn1]

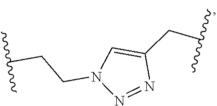 [SMILES: *CCn1cc(C*)nn1]

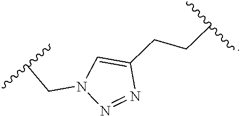 [SMILES: *CCc1cn(C*)nn1]

and

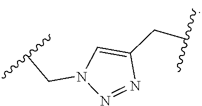 [SMILES: *Cc1cn(C*)nn1]

Protein Target Compounds (PTCs)

The PTCs of the present disclosure can be useful for modulating androgen receptor (AR). Further, the PTCs of the present disclosure can be useful for treating various diseases and conditions including, but not limited to, cancer. In some embodiments, the cancer is prostate cancer or breast cancer. In some embodiments, any of the PTCs disclosed herein can be a compound depicted as the compound before covalently attaching it to the LI.

In some embodiments, the present disclosure provides PTCs comprising the structure of formula (I):

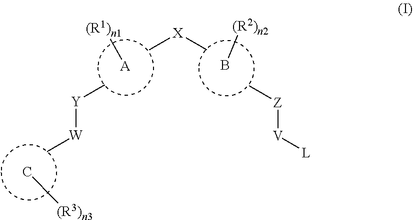

or a pharmaceutically acceptable salt, tautomer, stereoisomer or prodrug thereof, wherein:

A and B are each independently aryl or heteroaryl;

C is a 3- to 10-membered ring;

X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;

Y and Z are each independently a bond, —(CR⁸R⁹)ₘ—, —O—, —C(═O)—, —S—, —S(═O)—, —SO₂—, or —NR⁷—;

W and V are each independently a bond, —(CR^{8a}R^{9a})ₘ—, —C(═O)—, —N(R⁷)CO—, —CONR⁷—, or —NSO₂R⁷—;

L is hydrogen, halogen, —CF₂R¹⁰, —CF₃, —CN, —OR¹⁰; —NR¹¹R¹², or —CONR¹¹R¹²;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;

R⁸ and R⁹ are each independently hydrogen, halogen, or C₁-C₃ alkyl;

R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;

R⁷, R¹⁰ and R¹⁶ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R⁷ and R^{8a} taken together form an optionally substituted heterocyclyl;

R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or (R¹¹ and R¹²) or (R¹⁴ and R¹⁵) taken together form an optionally substituted heterocyclyl;

each m is independently 0, 1 or 2;

n1 and n2 are each independently 0, 1, 2, 3, or 4;

n3 is 0, 1, 2, 3, 4 or 5;

each t is independently 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (IA):

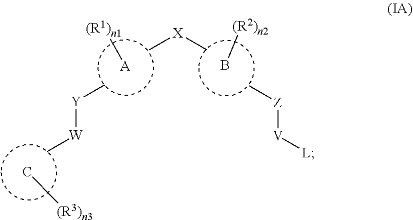

or a pharmaceutically acceptable salt, tautomer, stereoisomer or prodrug thereof, wherein:

A and B are each independently aryl or heteroaryl;

C is a 3- to 10-membered ring;

X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;

Y and Z are each independently a bond, —(CR⁸R⁹)ₘ—, —O—, —C(═O)—, —S—, —S(═O)—, —SO₂—, or —NR⁷—;

W and V are each independently a bond, —(CR^{8a}R^{9a})ₘ—, —C(═O)—, —N(R⁷)CO—, —CONR⁷—, or —NSO₂R⁷—;

L is hydrogen, halogen, —CF₂R¹⁰, —CF₃, —CN, —OR¹⁰; —NR¹¹R¹², or —CONR¹¹R¹²;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;

R⁸ and R⁹ are each independently hydrogen, halogen, or C₁-C₃ alkyl;

R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;

R⁷, R¹⁰ and R¹⁶ are each independently hydrogen, optionally substituted C₁-C₆ alkyl, optionally substituted C₂-C₆ alkenyl, optionally substituted C₂-C₆ alkynyl, C₁-C₆ haloalkyl, C₂-C₆ haloalkenyl, C₂-C₆ haloalkynyl, optionally substituted carbocyclyl, optionally substituted —CO(C₁-C₆ alkyl), —CO (optionally substituted heterocyclyl), optionally substituted heterocyclyl, optionally substituted aryl, or optionally substituted heteroaryl; or R⁷ and R^{8a} taken together form an optionally substituted heterocyclyl;

R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) taken together form an optionally substituted heterocyclyl;

each m is independently 0, 1 or 2;

n1 and n2 are each independently 0, 1, 2, 3, or 4;

n3 is 0, 1, 2, 3, 4 or 5;

each t is independently 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (IB):

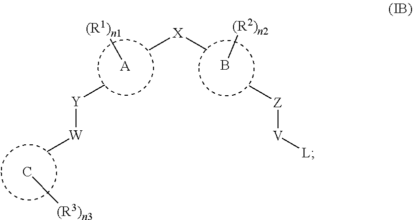

or a pharmaceutically acceptable salt, tautomer, stereoisomer or prodrug thereof, wherein:

A and B are each independently aryl or heteroaryl;

C is a 3- to 10-membered ring;

X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;

Y is a bond, —(CR⁸R⁹)ₘ—, —O—, —S—, —S(═O)—, —SO₂—, or —NR⁷—;

W is a bond, —(CR^{8a}R^{9a})ₘ—, —N(R⁷)CO—, —CONR⁷—, or —NSO₂R⁷—;

Z is a bond, —CH₂—, —C(CH₃)H—, —O—, —S—, —NH—, —NCH₃—, or —N(COCH₃)—;

V is —CH₂—, —CH₂CH₂—, —CH(CH₃)CH₂—, —CH₂CH(CH₃)—, or —CH₂CH₂CH₂—;

L is hydrogen, halogen, —CF₂R¹⁰, —CF₃, —CN, —OR¹⁰; —NR¹¹R¹², or —CONR¹¹R¹²;

R¹ and R² are each independently hydrogen, deuterium, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;

R⁸ and R⁹ are each independently hydrogen, halogen, or C₁-C₃ alkyl;

R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;

R⁷, R¹⁰ and R¹⁶ are each independently hydrogen, optionally substituted C₁-C₆ alkyl, optionally substituted C₂-C₆ alkenyl, optionally substituted C₂-C₆ alkynyl, optionally substituted C₁-C₆ alkyl-NH₂, C₁-C₆ haloalkyl, C₂-C₆ haloalkenyl, C₂-C₆ haloalkynyl, optionally substituted carbocyclyl, optionally substituted —CO(C₁-C₆ alkyl), —CO(optionally substituted heterocyclyl), optionally substituted heterocyclyl, optionally substituted aryl, or optionally substituted heteroaryl; or R⁷ and R^{8a} taken together form an optionally substituted heterocyclyl;

R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) or (R¹⁴ and R¹⁶) taken together form an optionally substituted heterocyclyl;

each m is independently 0, 1 or 2;

n1 and n2 are each independently 0, 1, 2, 3, or 4;

n3 is 0, 1, 2, 3, 4 or 5;

each t is independently 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising comprising the structure of formula (IC)

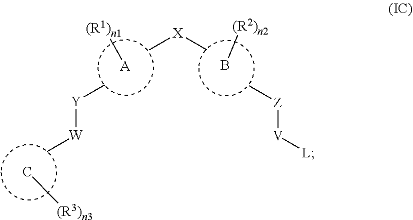

or a pharmaceutically acceptable salt, tautomer, stereoisomer or prodrug thereof, wherein:

A and B are each independently aryl or heteroaryl;

C is a 3- to 10-membered ring;

X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;

Y is a bond, —(CR⁸R⁹)ₘ—, —O—, —S—, —S(═O)—, —SO₂—, or —NR⁷—;

W is a bond, —(CR^{8a}R^{9a})ₘ—, —C(═O)—, N(R⁷)CO—, —CONR⁷—, or —NSO₂R⁷—;

Z is a bond, —(CR⁸R⁹)ₘ—, —O—, —S—, —S(═O)—, —SO₂—, or —NR⁷—;

V is —CH₂—, —CH₂CH₂—, —CH(CH₃)CH₂—, —CH₂CH(CH₃)—, or —CH₂CH₂CH₂—;

L is hydrogen, halogen, —CF₂R¹⁰, —CF₃, —CCl₂R¹⁰, —CCl₃, —CN, —OR¹⁰; —NR¹¹R¹², or —CONR¹¹R¹²;

R¹ and R² are each independently hydrogen, deuterium, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;

R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹³R¹⁴, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;

R⁸ and R⁹ are each independently hydrogen, halogen, or C₁-C₃ alkyl;

R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;

R⁷, R¹⁰ and R¹⁶ are each independently hydrogen, optionally substituted C₁-C₆ alkyl, optionally substituted C₂-C₆ alkenyl, optionally substituted C₂-C₆ alkynyl, optionally substituted C₁-C₆ alkyl-NH₂, C₁-C₆ haloalkyl, C₂-C₆ haloalkenyl, C₂-C₆ haloalkynyl, optionally substituted carbocyclyl, optionally substituted —CO(C₁-C₆ alkyl), —CO(optionally substituted heterocyclyl), optionally substituted heterocyclyl, optionally substituted aryl, or optionally substituted heteroaryl; or R⁷ and R^{8a} taken together form an optionally substituted heterocyclyl;

R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) or (R¹⁴ and R¹⁶) taken together form an optionally substituted heterocyclyl;

each m is independently 0, 1 or 2;

n1 and n2 are each independently 0, 1, 2, 3, or 4;

n3 is 0, 1, 2, 3, 4 or 5;

each t is independently 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (II):

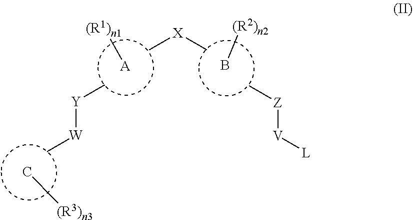

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —(CR⁸R⁹)ₘ—, —O—, —S—, —S(═O)—, —SO₂—, or —NR⁷—;

W is a bond, —CH₂—, —C(CH₃)H—, —C(═O)—, —NHCO—, —N(C₁-C₃ alkyl)CO—, or —CONH—, or —CON(C₁-C₃ alkyl)-;

V is a bond, —(CR^{8a}R^{9a})ₘ—, —C(═O)—, —N(R⁷)CO—, —CONR⁷—, or —NSO₂R⁷—;

L is hydrogen, halogen, —CF₂R¹⁰, —CF₃, —CN, —OR¹⁰; —NR¹¹R¹², or —CONR¹¹R¹²;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁸ and R⁹ are each independently hydrogen, halogen, or C₁-C₃ alkyl;

R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;

R¹¹, R¹², R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R⁷, R¹⁰ and R¹⁶ are each independently hydrogen, C₁-C₆ alkyl, C₂-C₆ alkenyl, or C₂-C₆ alkynyl;

each m is independently 0, 1 or 2;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (IIA):

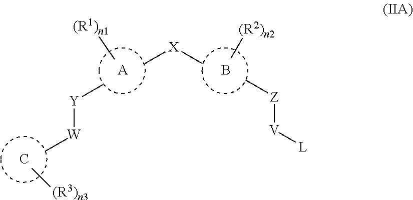

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —(CR⁸R⁹)ₘ—, —O—, —S—, —S(═O)—, —SO₂—, or —NR⁷—;

W is a bond, —CH₂—, —C(CH₃)H—, —NHCO—, —N(C₁-C₃ alkyl)CO—, or —CONH—, or —CON(C₁-C₃ alkyl)-;

V is —CH₂—, —CH₂CH₂—, —CH(CH₃)CH₂—, —CH₂CH(CH₃)—, or —CH₂CH₂CH₂—;

L is hydrogen, halogen, —CF₂R¹⁰, —CF₃, —CN, —OR¹⁰; —NR¹¹R¹², or —CONR¹¹R¹²;

R¹ and R² are each independently hydrogen, deuterium, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁸ and R⁹ are each independently hydrogen, halogen, or C₁-C₃ alkyl;

R¹¹, R¹², R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R⁷, R¹⁰ and R¹⁶ are each independently hydrogen, C₁-C₆ alkyl, C₂-C₆ alkenyl, C₂-C₆ alkynyl or C₁-C₆ alkyl-NH₂; or R¹⁴ and R¹⁶ taken together form a 3- to 6-membered heterocyclyl;

each m is independently 0, 1 or 2;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (IIB):

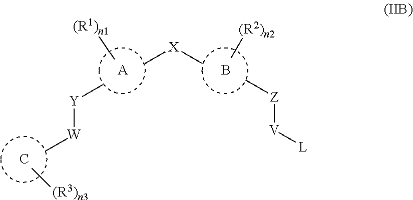

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —(CR⁸R⁹)ₘ—, —O—, —S—, —S(═O)—, —SO₂—, or —NR⁷—;

W is a bond, —CH₂—, or —C(CH₃)H—;

V is a bond, —(CR^{8a}R^{9a})ₘ—, —C(═O)—, —N(R⁷)CO—, —CONR⁷—, or —NSO₂R⁷—;

L is hydrogen, halogen, —CF₂R¹⁰, —CF₃, —CN, —OR¹⁰; —NR¹¹R¹², or —CONR¹¹R¹²;

R¹ and R² are each independently hydrogen, deuterium, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁸ and R⁹ are each independently hydrogen, halogen, or C₁-C₃ alkyl;

R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;

R¹¹, R¹², R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R⁷, R¹⁰ and R¹⁶ are each independently hydrogen, C₁-C₆ alkyl, C₂-C₆ alkenyl, C₂-C₆ alkynyl or C₁-C₆ alkyl-NH₂; or R¹⁴ and R¹⁶ taken together form a 3- to 6-membered heterocyclyl;

each m is independently 0, 1 or 2;

n1 and n2 are each independently 0, 1, or 2;

n3 is 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (III):

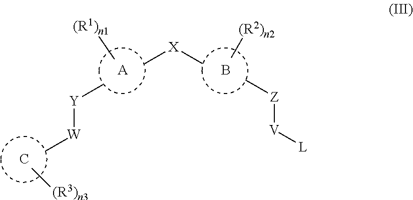

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷—;

Y is a bond, —(CR⁸R⁹)ₘ—, —O—, —S—, —S(═O)—, —SO₂—, or —NR⁷—;

W is a bond, —(CR^{8a}R^{9a})ₘ—, —C(═O)—, —N(R⁷)CO—, —CONR⁷—, or —NSO₂R⁷—;

Z is a bond, —CH₂—, —C(CH₃)H—, —O—, —S—, —NH—, —NCH₃—, or —N(COCH₃)—;

V is —CH₂—, —CH₂CH₂—, or —CH₂CH₂CH₂—;

L is halogen, —NH₂, or —CF₃;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is hydrogen, C₁-C₆ alkyl, C₂-C₆ alkenyl, or C₂-C₆ alkynyl;

R⁸ and R⁹ are each independently hydrogen, halogen, or C₁-C₃ alkyl;

R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;

R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R¹⁶ is hydrogen, C₁-C₃ alkyl, C₂-C₃ alkenyl, or C₂-C₃ alkynyl;

each m is independently 0, 1 or 2;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (IIIA):

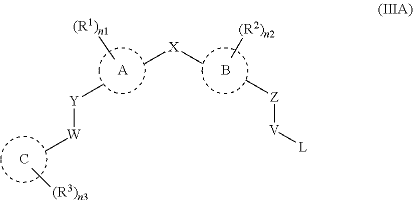

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷;

Y is a bond, —(CR⁸R⁹)ₘ—, —O—, —S—, —S(═O)—, —SO₂—, —NR⁷— or —N(COCH₃)—;

W is a bond, —(CR^{8a}R^{9a})ₘ—, —C(═O)—, —N(R⁷)CO—, —CONR⁷—, or —NSO₂R⁷—;

Z is a bond, —CH₂—, —C(CH₃)H—, —O—, —S—, —NH—, —NCH₃—, or —N(COCH₃)—;

V is —CH₂— and L is halogen, —NH₂, —CHCl₂, —CCl₃, or —CF₃; or

V is —CH₂CH₂— and L is halogen or —NH₂;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is hydrogen, C₁-C₆ alkyl, C₂-C₆ alkenyl, or C₂-C₆ alkynyl;

R⁸ and R⁹ are each independently hydrogen, halogen, or C₁-C₃ alkyl;

R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;

R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R¹⁶ is hydrogen, optionally substituted C₁-C₃ alkyl, optionally substituted C₂-C₃ alkenyl, optionally substituted C₂-C₃ alkynyl, C₃-C₆ cycloalkyl, or phenyl;

each m is independently 0, 1 or 2;

n1 and n2 are each independently 0, 1, or 2;

n3 is 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (IV):

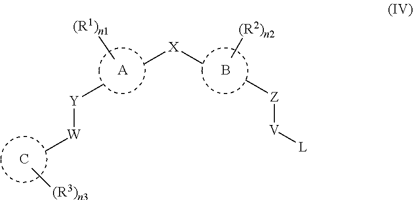

or a pharmaceutically acceptable salt, tautomer, stereoisomer or prodrug thereof, wherein:

A and B are each independently selected from phenyl, pyridyl, pyrimidyl, or thiophene;

C is a 3- to 10-membered ring;

X is a bond, —(CR⁵R⁶)ₜ—, or —NR⁷—;

Y and Z are each independently a bond, —CH₂—, —C(CH₃)H—, —O—, —S—, —NH—, —NCH₃—, or —N(COCH₃)—;

W is a bond, —CH₂—, —C(CH₃)H—, —C(═O)—, —N(R⁷)CO—, or —CONR⁷—;

V is —CH₂—, —CH₂CH₂—, or —CH₂CH₂CH₂—;

L is halogen, —NH₂, or —CF₃;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is H or C₁-C₆ alkyl;

R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R¹⁶ is hydrogen, C₁-C₃ alkyl, C₂-C₃ alkenyl, or C₂-C₃ alkynyl;

n1 and n2 are each independently 0, 1, or 2;

n3 is 0, 1, 2, 3, 4 or 5; and

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (V):

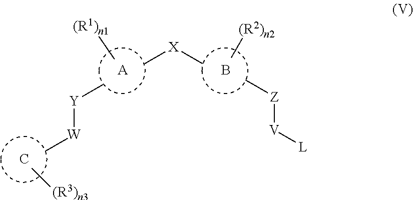

or a pharmaceutically acceptable salt, tautomer, stereoisomer or prodrug thereof, wherein:

A and B are each independently selected from phenyl, pyridyl, pyrimidyl, or thiophene;

C is a 5- to 10-membered heteroaryl or aryl;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —CH₂—, —C(CH₃)H—, —O—, —S—, —NH—, —NCH₃—, or —N(COCH₃)—;

W is a bond, —CH₂—, —C(CH₃)H—, —C(═O)—, —N(R⁷)CO—, or —CONR⁷—;

V is —CH₂—, —CH₂CH₂—, or —CH₂CH₂CH₂—;

L is halogen, —NH₂, or —CF₃;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, C₁-C₃ alkoxy; or R⁵ and R⁶ taken together form an optionally substituted 3- to 6-membered carbocyclyl or heterocyclyl;

R⁷ is H or C₁-C₆ alkyl;

R¹³, R¹⁴ and R¹⁵ are each independently hydrogen, C₁-C₃ alkyl, C₂-C₃ alkenyl, C₂-C₃ alkynyl; or R¹⁴ and R¹⁵ taken together form a 3- to 6-membered heterocyclyl;

R¹⁶ is hydrogen, C₁-C₃ alkyl, C₂-C₃ alkenyl, or C₂-C₃ alkynyl;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (VA):

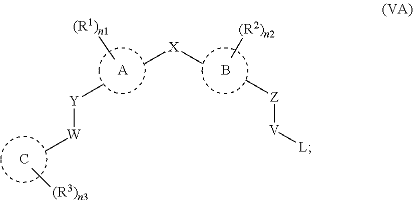

or a pharmaceutically acceptable salt, tautomer, stereoisomer or prodrug thereof, wherein:

A and B are each independently selected from phenyl, pyridyl, pyrimidyl, or thiophene;

C is a 5- to 10-membered heteroaryl or aryl;

X is a bond, —(CR⁵R⁶)ₜ—, or —NR⁷;

Y is a bond, —CH₂—, —C(CH₃)H—, —O—, —S—, —NH—, —NCH₃—, or —N(COCH₃)—;

Z is a bond, —CH₂—, —C(CH₃)H—, —O—, —S—, —NH—, —NCH₃—, or —N(COCH₃)—;

W is a bond, —CH₂—, —C(CH₃)H—, —C(═O)—, —N(R⁷)CO—, or —CONR⁷—;

V is —CH₂—, —CH₂CH₂—, —CH(CH₃)CH₂—, —CH₂CH(CH₃)—, or —CH₂CH₂CH₂—;

L is hydrogen, halogen, —OH, —NH₂, or —CF₃;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —NR¹⁴CONR¹⁴R¹⁵, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, —NH₂, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is H, C₁-C₆ alkyl, —CO(C₁-C₆ alkyl);

R¹³, R¹⁴ and R¹⁵ are each independently hydrogen, C₁-C₃ alkyl, C₂-C₃ alkenyl, C₂-C₃ alkynyl, or —COO(C₁-C₆ alkyl); or R¹⁴ and R¹⁵ taken together form a 3- to 6-membered heterocyclyl;

R¹⁶ is hydrogen, C₁-C₃ alkyl, C₁-C₃ haloalkyl, C₂-C₃ alkenyl, or C₂-C₃ alkynyl;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (VI):

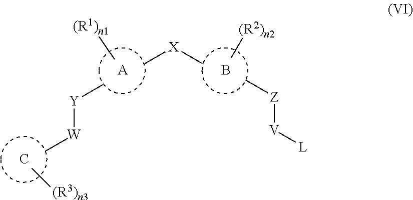

or a pharmaceutically acceptable salt, tautomer, stereoisomer or prodrug thereof, wherein:

A and B are each independently selected from phenyl, pyridyl, pyrimidyl, or thiophene;

C is a 5- to 10-membered heterocyclyl;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —CH₂—, —C(CH₃)H—, —O—, —S—, —NH—, —NCH₃—, or —N(COCH₃)—;

W is a bond, —CH₂—, —C(CH₃)H—, —C(═O)—, —N(R⁷)CO—, or —CONR⁷—;

V is —CH₂—, —CH₂CH₂—, or —CH₂CH₂CH₂—;

L is halogen, —NH₂, or —CF₃;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is H or C₁-C₆ alkyl;

R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R¹⁶ is hydrogen, C₁-C₃ alkyl, C₂-C₃ alkenyl, or C₂-C₃ alkynyl;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (A):

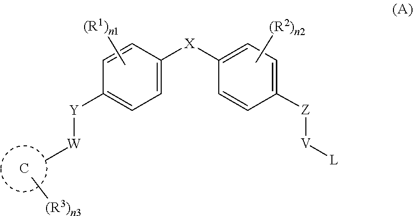

or a pharmaceutically acceptable salt, tautomer, stereoisomer or prodrug thereof, wherein:

C is a phenyl or a 5- to 7-membered monocyclic heteroaryl comprising 1, 2, or 3 heteroatoms selected from O, S, or N as a ring member;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —CH₂—, —O—, or —NH—;

W is a bond, —CH₂—, —C(CH₃)H—, —C(═O)—, —N(R⁷)CO—, or —CONR⁷—;

V is —CH₂—, —CH₂CH₂—, or —CH₂CH₂CH₂—;

L is halogen, —NH₂, or —CF₃;

R¹ and R² are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, —S(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);

R⁵ and R⁶ are each independently hydrogen, halogen, —OH, or C₁-C₃ alkyl;

R⁷ is H or C₁-C₆ alkyl;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (B)

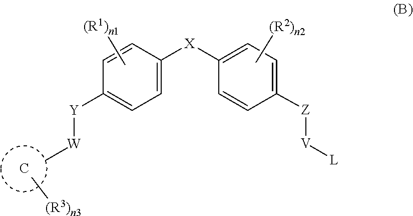

or a pharmaceutically acceptable salt, tautomer, stereoisomer or prodrug thereof, wherein:

C is a 5- to 7-membered saturated or partially saturated monocyclic heterocycle comprising 1, 2, or 3 heteroatoms selected from O, S, or N as a ring member;

X is a bond, —(CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —CH₂—, —O—, or —NH—;

W is a bond, —CH₂—, —C(CH₃)H—, —C(═O)—, —N(R⁷)CO—, or —CONR⁷—;

V is —CH₂—, —CH₂CH₂—, or —CH₂CH₂CH₂—;

L is halogen, —NH₂, or —CF₃;

R¹ and R² are each independently hydrogen, halogen, —CN, —CF₃, methyl, or —CONH₂;

R³ is selected from hydrogen, F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);

R⁵ and R⁶ are each independently hydrogen, halogen, —OH, or C₁-C₃ alkyl;

R⁷ is H or C₁-C₆ alkyl;

R¹⁶ is hydrogen or C₁-C₃ alkyl;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment of the PTCs of formula (I), (IC), (II), (III), (IIIA), (IV), (V), (VI), (A), or (B), —V-L is —CH₂CH₂Cl, —CH₂CH₂CH₂Cl, —CH₂CH₂NH₂, or —CH₂CH₂CH₂NH₂.

In one embodiment of the PTCs of formula (I), (IC), (II), (III), (IIIA), (IV), (V), (VI), (A), or (B), —Y—W— is a bond, —OCH₂—, —OCH₂CH₂—, —OCH(CH₃)—, —NH—, —NHCH₂—, —NHC(═O)—, or —C(═O)NH—.

In one embodiment of the PTCs of formula (I), (IC), (II), (III), (IIIA), (IV), (V), (VI), (A), or (B), X is a bond, —CH₂—, —C(CH₃)H—, —C(CH₃)₂—, or —CH₂CH₂—.

In one embodiment, the present disclosure provides PTCs comprising the structure of formula (C):

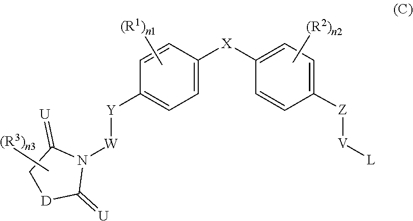

or a pharmaceutically acceptable salt, tautomer, stereoisomer or prodrug thereof, wherein:

A and B are each independently selected from phenyl, pyridyl, pyrimidyl, or thiophene;

X is a bond, (CR⁵R⁶)ₜ—, or —NR⁷—;

Y is a bond, —CH₂—, —C(CH₃)H—, —O—, —S—, —NH—, —NCH₃—, or —N(COCH₃)—;

Z is a bond, —CH₂—, —C(CH₃)H—, —O—, —S—, —NH—, —NCH₃—, or —N(COCH₃)—;

W is a bond, —CH₂—, —C(CH₃)H—, —C(═O)—, —N(R⁷)CO—, or —CONR⁷—;

V is —CH₂—, —CH₂CH₂—, or —CH₂CH₂CH₂—;

L is halogen, —NH₂, or —CF₃;

D is —NH or —NR³;

U is each independently O, S, or NR¹⁶;

R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from hydrogen, halogen, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);

R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;

R⁷ is H or C₁-C₆ alkyl;

R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;

R¹⁶ is hydrogen, C₁-C₃ alkyl, C₂-C₃ alkenyl, or C₂-C₃ alkynyl;

n1 and n2 are each independently 0, 1, or 2;

n3 is 0, 1, 2, or 3;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (D):

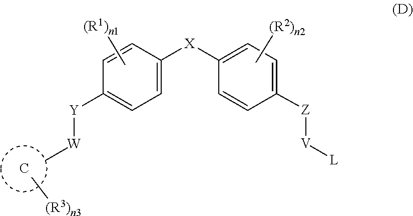

or a pharmaceutically acceptable salt, tautomer, stereoisomer or prodrug thereof, wherein:

C is a 5- or 6-membered heteroaryl comprising 1 or 2 heteroatoms selected from O, S, or N as a ring member;

X is —(CR⁵R⁶)ₜ— or —NR⁷—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is hydrogen, halogen, —NH₂, or —CF₃;

R¹ and R² are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, —S(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);

R⁵ and R⁶ are each independently hydrogen, halogen, —OH, or C₁-C₃ alkyl;

R⁷ is H or C₁-C₆ alkyl;

n1 and n2 are each independently 0, 1, or 2;

n3 is 0, 1, 2, 3, 4 or 5;

t is 0, 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (E):

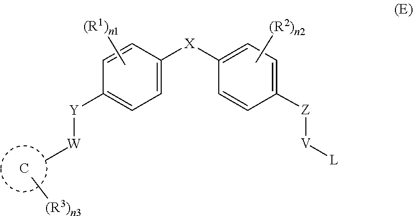

or a pharmaceutically acceptable salt, tautomer, stereoisomer or prodrug thereof, wherein:

C is

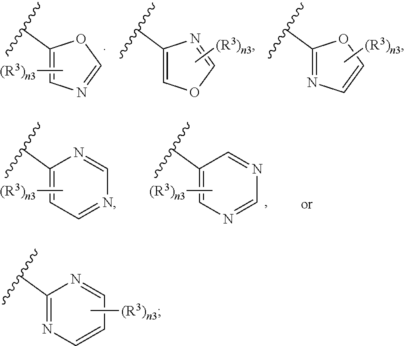

X is —(CR⁵R⁶)ₜ— or —NR⁷—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is hydrogen or halogen;

R¹ and R² are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, —S(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);

R⁵ and R⁶ are each independently hydrogen or C₁-C₃ alkyl;

R⁷ is H or C₁-C₆ alkyl;

n1 and n2 are each independently 0, 1, or 2;

n3 is 0, 1, or 2;

t is 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-I):

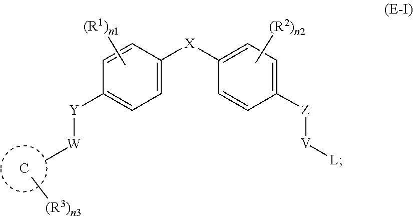

or a pharmaceutically acceptable salt, tautomer, stereoisomer or prodrug thereof, wherein:

C is

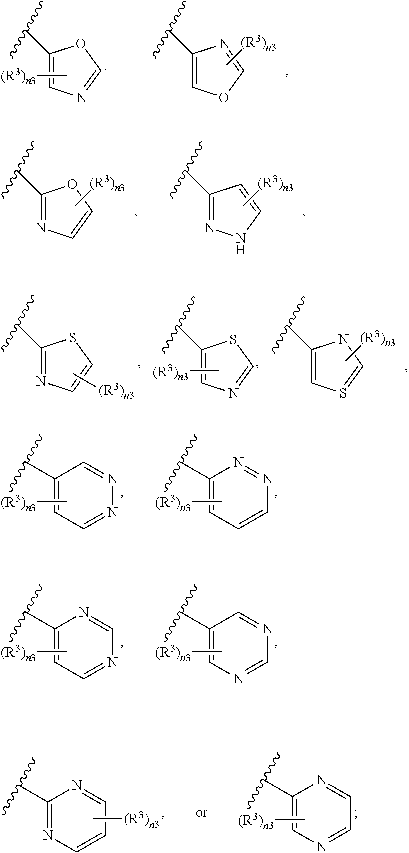

X is —(CR⁵R⁶)ₜ— or —NR⁷—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, —CH₂CH₂CH₂—, or —CH₂CHClCH₂—;

L is hydrogen, —OH, or halogen;

R¹ and R² are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

R⁷ is H or C₁-C₆ alkyl;

n1 and n2 are each independently 0, 1, or 2;

n3 is 0, 1, or 2;

t is 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment of the PTCs of formula (A)-(C) or (E-1), R³ is selected from hydrogen, F, Cl, Br, I, —CN, —CF₃, —OH, methyl, methoxy, —S(C₁-C₃ alkyl), —SO₂(C₁-C₃ alkyl), —NH₂, —NHSO₂CH₃, —NHSO₂CF₃, —N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —NHCO(C₁-C₃ alkyl).

In one embodiment, the present disclosure provides PTCs comprising the structure of formula (F):

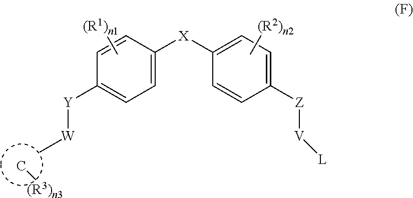

or a pharmaceutically acceptable salt, tautomer, stereoisomer or prodrug thereof, wherein:

C is

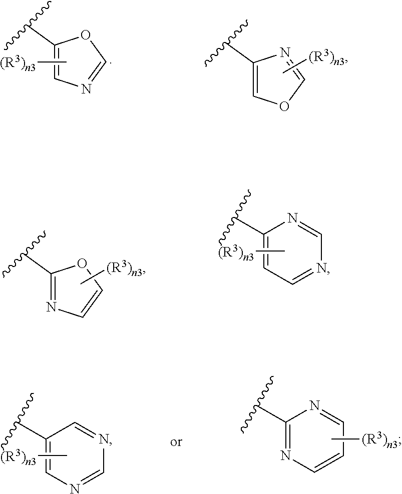

X is —(CR⁵R⁶)ₜ— or —NR⁷—;

Y is —O—;

Z is —O—;

W is —CH₂— or —C(CH₃)H—;

V is —CH₂—, —CH₂CH₂—, or —CH₂CH₂CH₂—;

L is hydrogen or halogen;

R¹ and R² are each independently halogen or —CN;

R³ is selected from —NHSO₂CH₃, —N(CH₃)SO₂CH₃, or —SO₂CH₃;

R⁵ and R⁶ are each independently hydrogen or C₁-C₃ alkyl;

R⁷ is H or C₁-C₆ alkyl;

n1 and n2 are each independently 0, 1, or 2;

n3 is 0, 1, or 2;

t is 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (G):

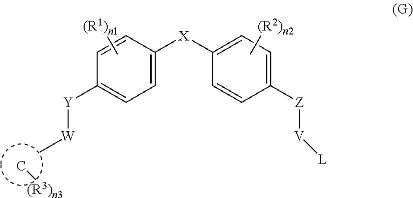

or a pharmaceutically acceptable salt, tautomer, stereoisomer or prodrug thereof, wherein:

C is

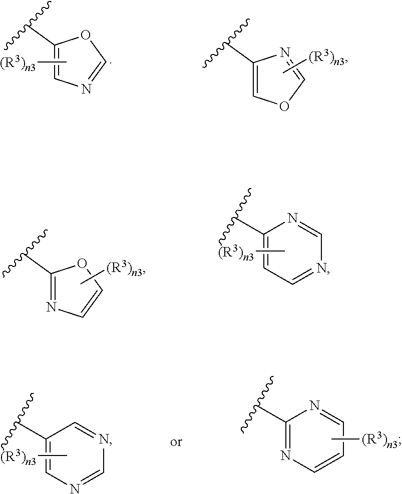

X is —(CR⁵R⁶)ₜ— or —NR⁷—;

Y is —O—;

Z is —O—;

W is —CH₂— or —C(CH₃)H—;

V is —CH₂— and L is hydrogen; or alternatively, V is —CH₂CH₂— or —CH₂CH₂CH₂—, and L is halogen;

R¹ and R² are each independently Cl or —CN;

R³ is selected from —NHSO₂CH₃, —N(CH₃)SO₂CH₃, or —SO₂CH₃;

R⁵ and R⁶ are each independently hydrogen or methyl;

R⁷ is H or C₁-C₆ alkyl;

n1 and n2 are each independently 0, 1, or 2;

n3 is 1, or 2;

t is 1; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of (G-I)

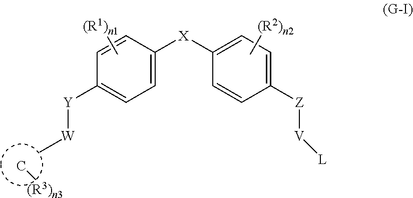

or a pharmaceutically acceptable salt, tautomer, stereoisomer or prodrug thereof, wherein:

C is

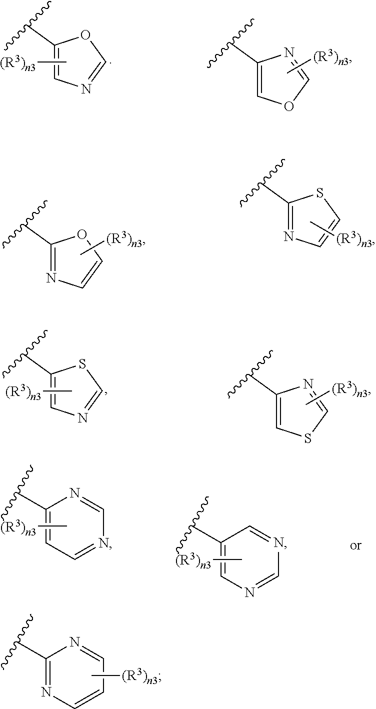

X is —(CR⁵R⁶)ₜ—;

Y is —O—;

Z is —O—;

W is —CH₂— or —C(CH₃)H—;

V is —CH₂CH₂— or —CH₂CH₂CH₂—;

L is halogen;

R¹ and R² are each independently Cl or —CN;

R³ is selected from —NHSO₂CH₃, —N(CH₃)SO₂CH₃, or —SO₂CH₃;

R⁵ and R⁶ are each independently hydrogen or methyl;

n1 and n2 are each independently 0, 1, or 2;

n3 is 1 or 2;

t is 1; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (G-II)

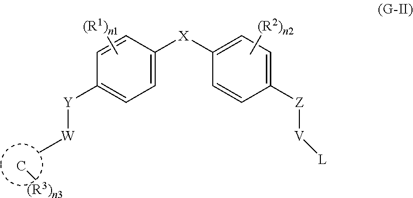

or a pharmaceutically acceptable salt, tautomer, stereoisomer or prodrug thereof, wherein:

C is

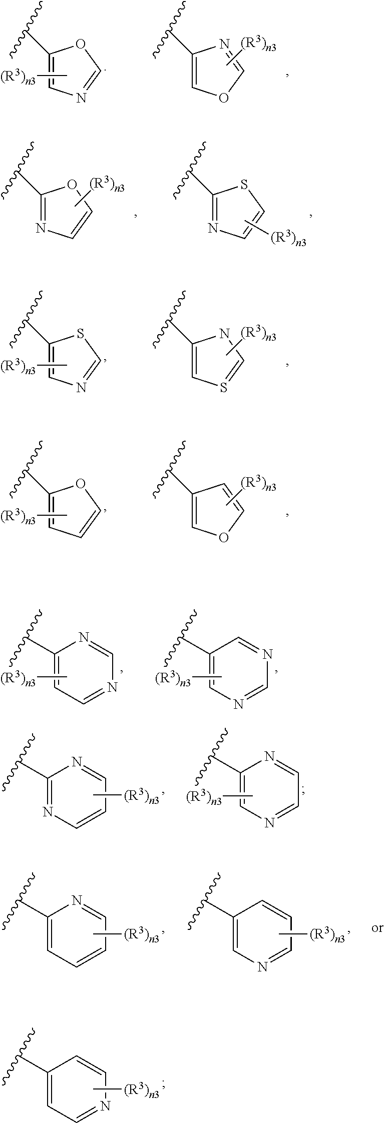

X is —(CR⁵R⁶)ₜ—;

Y is —O—;

Z is —O—;

W is —CH₂— or —C(CH₃)H—;

V is —CH₂CH₂—;

L is halogen;

R¹ and R² are each independently Cl or —CN;

at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or methyl;

n1 and n2 are each independently 0, 1, or 2;

n3 is 1 or 2;

t is 1; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment, the present disclosure provides PTCs comprising the structure of formula (H):

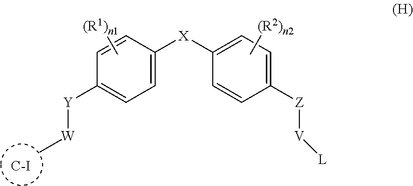

or a pharmaceutically acceptable salt, tautomer, stereoisomer or prodrug thereof, wherein:

C-I is

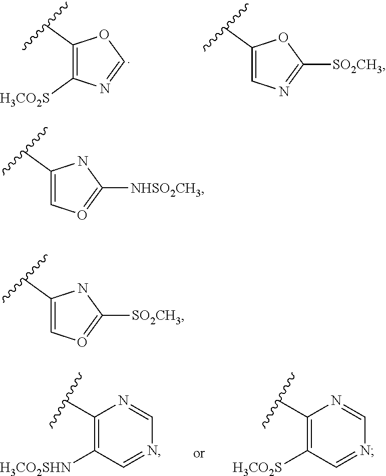

X is —(CR⁵R⁶)ₜ— or —NR⁷—;

Y is —O—;

Z is —O—;

W is —CH₂— or —C(CH₃)H—;

V is —CH₂—, —CH₂CH₂— or —CH₂CH₂CH₂—;

L is halogen;

R¹ and R² are each independently Cl or —CN;

R⁵ and R⁶ are each independently hydrogen or methyl;

R⁷ is H or C₁-C₆ alkyl;

n1 and n2 are each independently 0, 1, or 2;

t is 1; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (H-I):

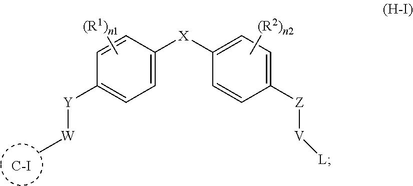

or a pharmaceutically acceptable salt, tautomer, stereoisomer or prodrug thereof, wherein:

C-I is

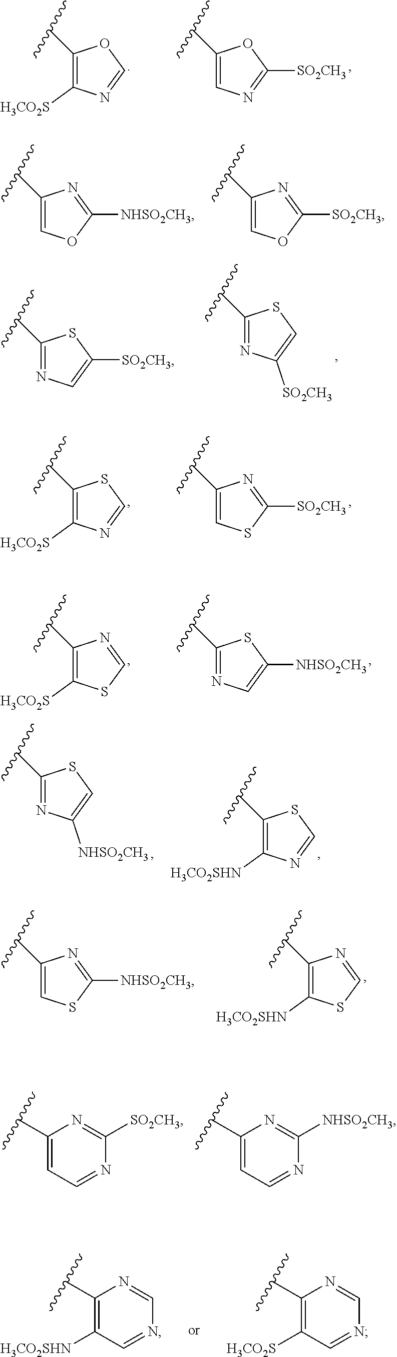

X is —(CR⁵R⁶)ₜ—;

Y is —O—;

Z is —O—;

W is —CH₂— or —C(CH₃)H—;

V is —CH₂—, —CH₂CH₂— or —CH₂CH₂CH₂—;

L is halogen;

R¹ and R² are each independently Cl or —CN;

R⁵ and R⁶ are each independently hydrogen or methyl;

R⁷ is H or C₁-C₆ alkyl;

n1 and n2 are each independently 0, 1, or 2;

t is 1; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-II):

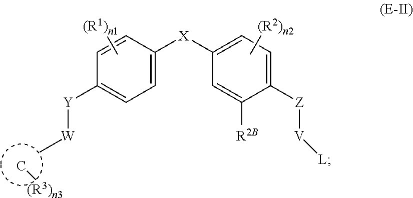

or a pharmaceutically acceptable salt, tautomer, stereoisomer or prodrug thereof, wherein:

C is

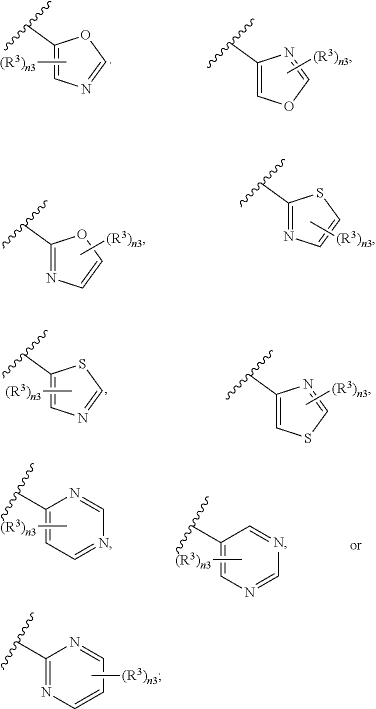

X is —(CR⁵R⁶)—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is halogen;

R¹, R^{2A} and R^{2B} are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

n1 is 0, 1, or 2;

n3 is 1 or 2;

wherein when C—R³ is

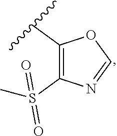 [SMILES: *c1ocnc1S(C)(=O)=O]

R^{2A} and R^{2B} are not both Cl; and
wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-III):

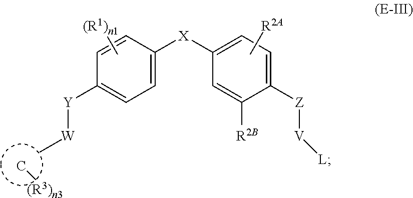

or a pharmaceutically acceptable salt, tautomer, stereoisomer or prodrug thereof, wherein:

C is

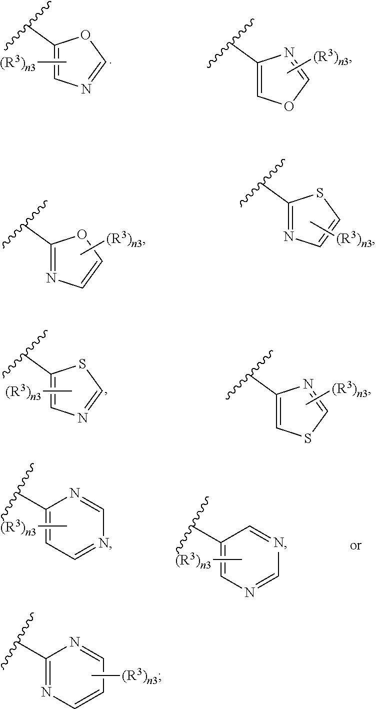

X is —(CR⁵R⁶)—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is halogen;

R¹, R^{2A} and R^{2B} are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

n1 is 0, 1, or 2;

n3 is 1 or 2;

wherein when C—R³ is

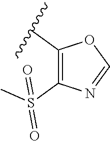 [SMILES: *c1ocnc1S(C)(=O)=O]

and one of R^{2A} and R^{2B} is Cl, then the other of R^{2A} and R^{2B} is not —CN; and
wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-IV):

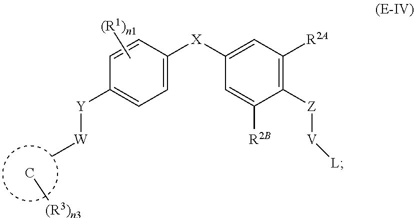

or a pharmaceutically acceptable salt, tautomer, stereoisomer or prodrug thereof, wherein:

C is

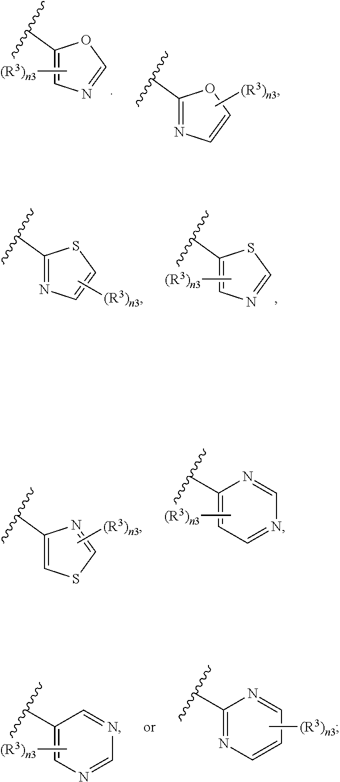

X is —(CR⁵R⁶)—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is halogen;

R¹, R^{2A} and R^{2B} are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

n1 is 0, 1, or 2;

n3 is 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-V):

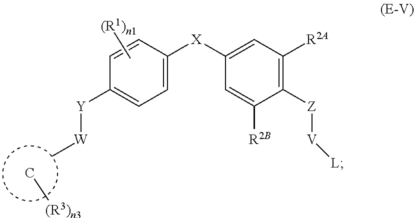

or a pharmaceutically acceptable salt, tautomer, stereoisomer or prodrug thereof, wherein:

C is

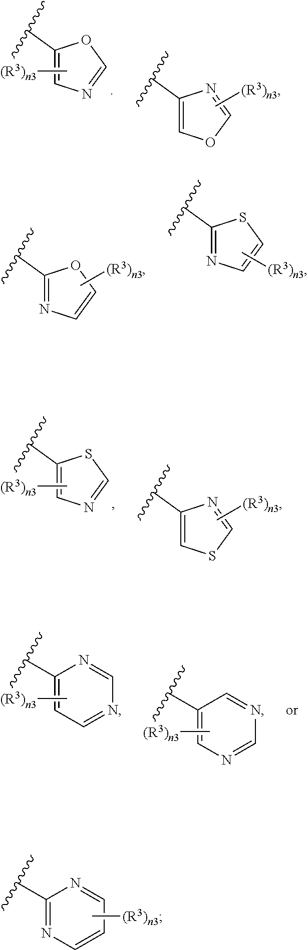

X is —(CR⁵R⁶)—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is halogen;

R¹, R^{2A} and R^{2B} are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

n1 is 0, 1, or 2;

n3 is 1 or 2;

wherein when C—R³ is

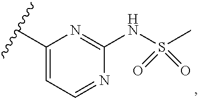 [SMILES: *c1ccnc(NS(C)(=O)=O)n1]

R^{2A} and R^{2B} are not both Cl; and
wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-VI):

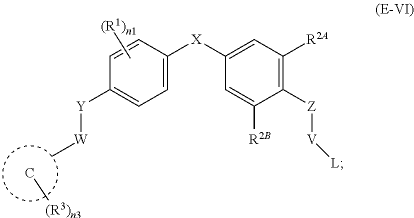

or a pharmaceutically acceptable salt, tautomer, stereoisomer or prodrug thereof, wherein:

C is

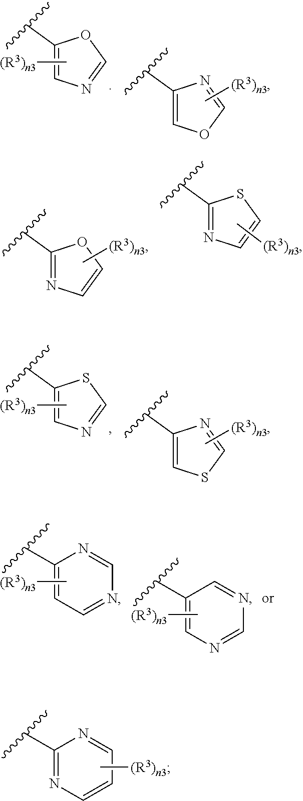

X is —(CR⁵R⁶)—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is halogen;

R¹, R^{2A} and R^{2B} are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

n1 is 0, 1, or 2;

n3 is 1 or 2;

wherein when C—R³ is

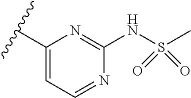 [SMILES: *c1ccnc(NS(C)(=O)=O)n1]

and one of R^{2A} and R^{2B} is Cl, then the other of R^{2A} and R^{2B} is not —CN; and
wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment the present disclosure provides PTCs comprising the structure of formula (E-VII):

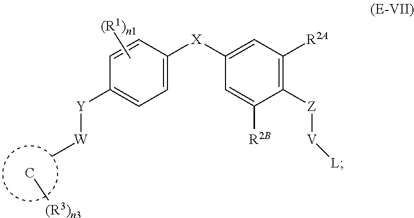

or a pharmaceutically acceptable salt, tautomer, stereoisomer or prodrug thereof, wherein:

C is

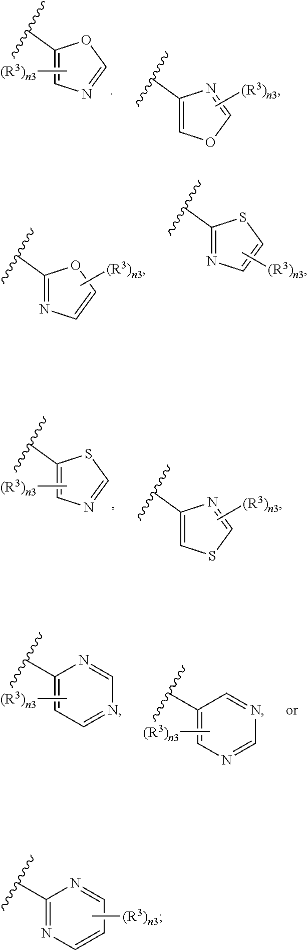

X is —(CR⁵R⁶)—;

Y is a bond, —CH₂—, —O—, or —NH—;

Z is a bond, —CH₂—, —O—, or —NH—;

W is a bond, —CH₂—, or —C(CH₃)H—;

V is —CH₂—, —CH₂CH₂—, or —CH₂CH₂CH₂—;

L is halogen;

R¹, R^{2A} and R^{2B} are each independently hydrogen, halogen, —CN, —CF₃, methyl, or —CONH₂;

R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);

R⁵ and R⁶ are each independently hydrogen or C₁-C₃ alkyl;

n1 is 0, 1, or 2;

n3 is 1 or 2; and

wherein one atom or one chemical group in the PTC is replaced to form a covalent bond to the LI. In some embodiments, one atom in the PTC is replaced to form a covalent bond to the LI. In some embodiments, one chemical group in the PTC is replaced to form a covalent bond to the LI.

In one embodiment of the PTCs of formula (I), (IA), (IB), (IC), (II), (IIA), (IIB), (III), (IIIA), (IV), (V), (VA), or (VI) (denoted as formula “(I)-(VI)”), A and B are each independently 5- or 6-membered aryl or heteroaryl. In one embodiment, A and B are each independently selected from phenyl, pyridyl, pyrimidyl, or thiophene. In one embodiment, A and B are each phenyl.

In another embodiment, A has a meta or para connectivity with X and Y. In some embodiments, B has a meta or para connectivity with X and Z.

In one embodiment of the PTCs of formula (I)-(VI), A and B are phenyl and has one of the connectivity as shown:

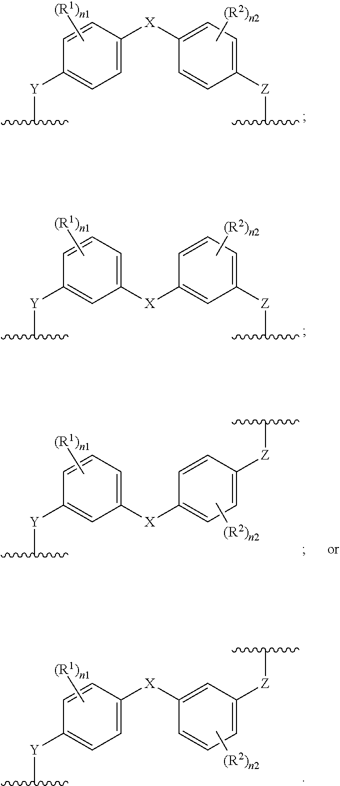

In one embodiment of the PTCs of formula (I)-(VA) and (A) (e.g., formula (I), (IA), (IB), (IC), (II), (IIA), (IIB), (III), (IIIA), (IV), (V) (VA), and (A)), C is aryl or heteroaryl. In some embodiments, C is 5- to 10-membered aryl or heteroaryl. In other embodiments, C is aryl. In some embodiments, C is phenyl or naphthyl. In other embodiments, C is aryl. In some embodiments, C is phenyl.

In one embodiment of the PTCs of formula (I)-(VA) and (A), C is heteroaryl. In one embodiment, C monocyclic or bicyclic heteroaryl. In another embodiment, C is monocyclic heteroaryl. In some embodiments, C is 5- or 10-membered heteroaryl. In some embodiments, C is 5- or 6-membered heteroaryl, which is optionally substituted with 1, 2, 3, 4, or 5 R³. In some embodiments, C is 5- or 6-membered heteroaryl containing 1, 2, or 3 heteroatoms selected from O, S, or N, wherein the heteroaryl is optionally substituted with 1, 2, 3, 4, or 5 R³. In some embodiments, C is 5- or 6-membered heteroaryl containing 1 or 2 heteroatoms selected from O, S, or N, wherein the heteroaryl is optionally substituted with 1, 2, 3, 4, or 5 R³.

In one embodiment of the PTCs of formula (I)-(VA), (A), or (D), C is pyrazole, imidazole, oxazole, oxadiazole, oxazolone, isoxazole, thiazole, pyridyl, or pyrimidyl, which are each optionally substituted with 1, 2, 3, 4, or 5 R³. In one embodiment, C, which is substituted with (R³)n3, is pyrazole, imidazole, oxazole, oxadiazole, oxazolone, isoxazole, thiazole, pyridyl, pyrazine, furan or pyrimidyl. In one embodiment, C is pyrazole, imidazole, oxazole, oxadiazole, oxazolone, isoxazole, thiazole, pyridyl, pyrazine, furan or pyrimidyl, which are each substituted with 1, 2, 3, 4, or 5 R³.

In one embodiment of the PTCs of formula (I)-(VA), (A), or (D), C is selected from

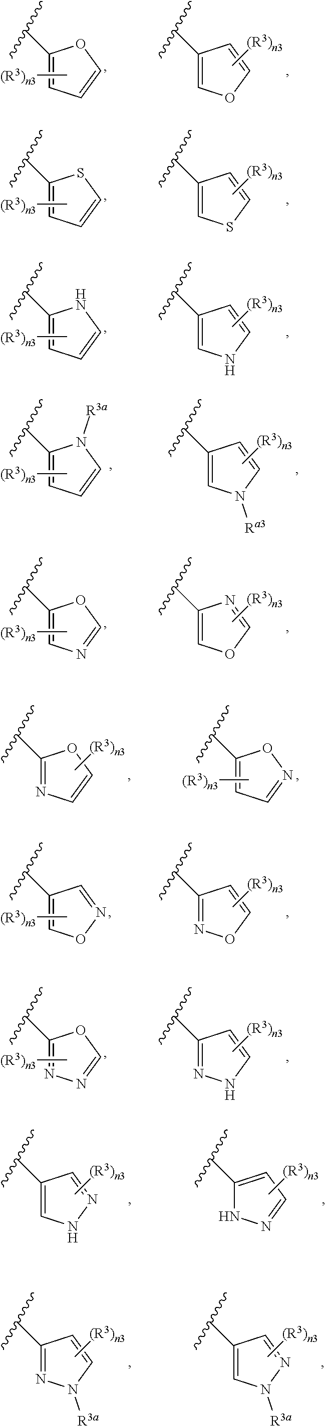

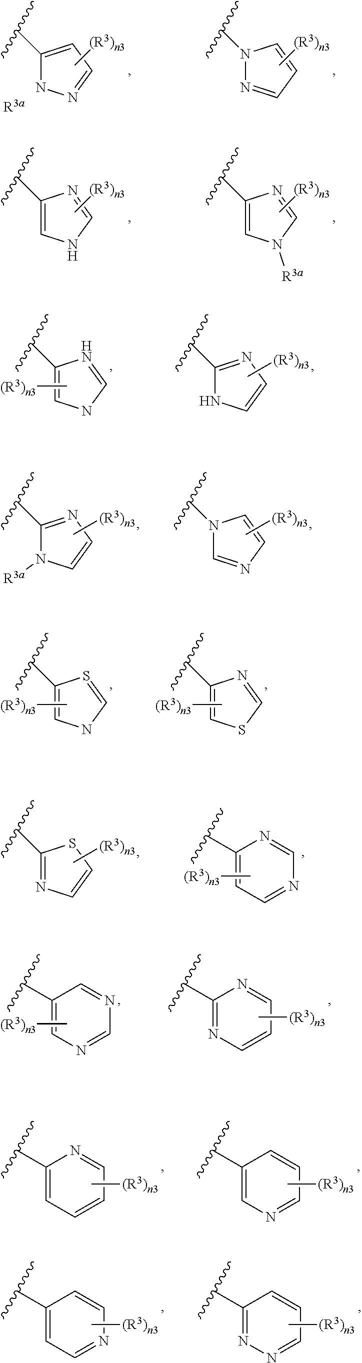

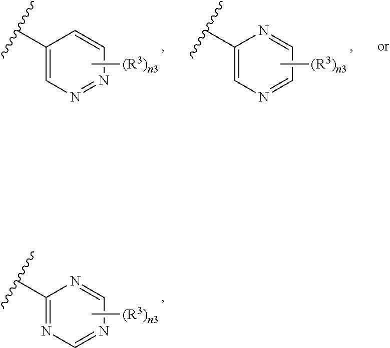

wherein R^{3a} is C₁-C₃ alkyl. In one embodiment of the PTCs of formula (I)-(VA), (A) or (D), C is selected from

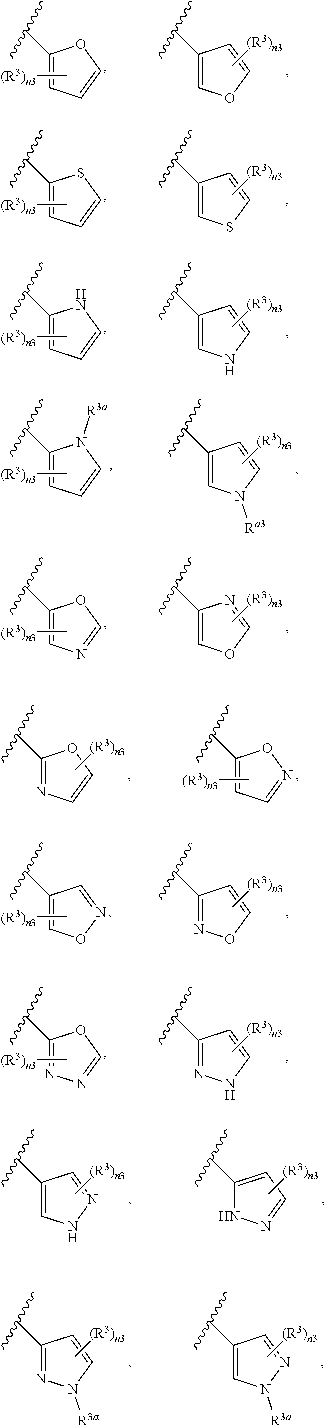

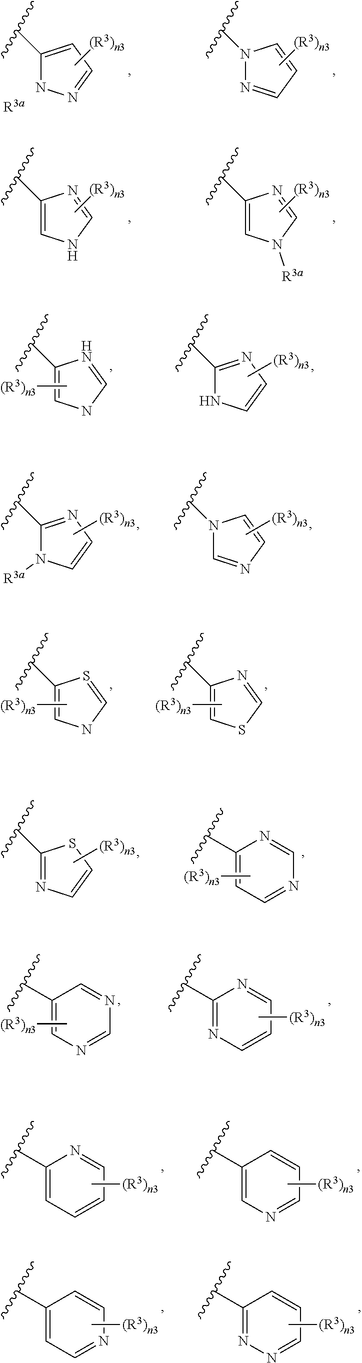

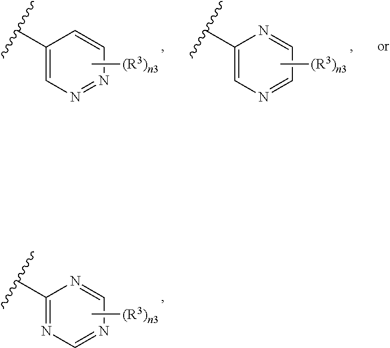

wherein R^{3a} is C₁-C₃ alkyl.

In one embodiment of the PTCs of formula (I)-(VA), (A), or (D), C is

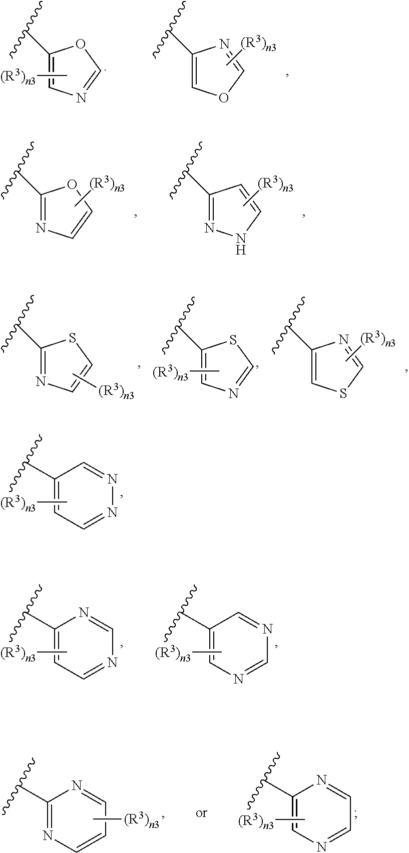

In one embodiment, C is

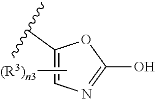

or in its tautomeric form

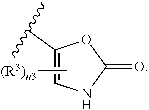

In one embodiment, C is

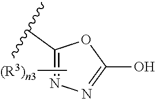

or in its tautomeric form

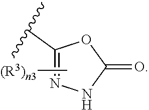

In one embodiment of the PTCs of formula (I)-(IV), C is heterocyclyl. In one embodiment, C is saturated or partially saturated heterocycle. In some embodiments, C is monocyclic or bicyclic. In some embodiments, C is 5- to 7-membered heterocyclyl comprising 1, 2, or 3 heteroatoms selected from O, S, or N as a ring member.

In one embodiment of the PTCs of formula (I)-(VA), (B) and (C), C is imidazolidine, imidazolidine-dione, or dihydrooxazole. In one embodiment, C is selected from

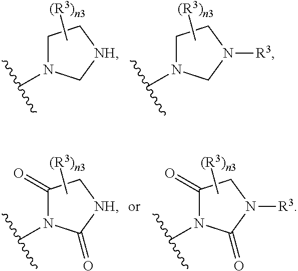

In one embodiment of the PTCs of formula (I)-(VA), (B) and (C), C is

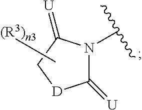

D is —O—, —NH— or —NR³—; and U is each independently O, S, or NR¹⁶. In one embodiment, D is —NH— or —NR³—. In some embodiments, at least one U is O. In other embodiments, each U is O. In some embodiments, at least one R³ is —SO₂CH₃, —NHSO₂CH₃, —CH₂NHSO₂CH₃, —SO₂NH₂, —CONH₂, or —NHCOCH₃.

In one embodiment of the PTCs of formula (I)-(VA), C is aryl. In some embodiments, C is phenyl or naphthyl. In one embodiment of the PTCs of formula (I)-(VA) or (A), C is phenyl.

In on embodiment PTCs of formula (I)-(VI), C is bicyclic heteroaryl or heterocyclyl. In one embodiment, C is

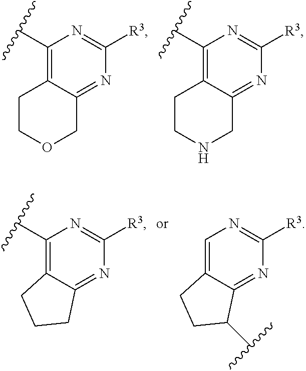

In one embodiment of the PTCs of formula (I), Z is a bond, —(CR⁸R⁹)ₘ—, —O—, —C(═O)—, —S—, —S(═O)—, —SO₂—, or —NR⁷—. In one embodiment, Z is —(CR⁸R⁹)ₘ—, —O—, —C(═O)—, —S—, —S(═O)—, —SO₂—, or —NR⁷—. In some embodiments, Z is —C(═O)—.

In one embodiment of the PTCs of formula (I)-(VI), (A), (B), and (C), Z is a bond, —CH₂—, —C(CH₃)H—, —O—, —S—, —NH—, —NCH₃—, or —N(COCH₃)—. In one embodiment, Z is —CH₂—, —C(CH₃)H—, —O—, —S—, —NH—, —NCH₃—, or —N(COCH₃)—. In some embodiments, Z is a bond, —CH₂—, —O—, or —NCH₃—. In some embodiments, Z is a bond, —CH₂—, —O—, or —NH—. In some embodiments of the PTCs of formula (I)-(VI) and (A)-(H-I), Z is —O—. As used herein, “PTCs of formula (I)-(IV) and (A)-(H-I)” refers to PTCs of formula (I), (IA), (IB), (IC), (II), (IIA), (IIIA), (IIB), (III), (IV), (IVA), (V), (VA), (VI), (A), (A-I), (B)-(D), (E), (E-I)-(E-VII), (F), (G), (G-I), (G-II), (H), and (H-I).

In one embodiment of the PTCs of formula (I), Y is a bond, —(CR⁸R⁹)ₘ—, —O—, —C(═O)—, —S—, —S(═O)—, —SO₂—, or —NR⁷—. In one embodiment, Y is —(CR⁸R⁹)ₘ—, —O—, —C(═O)—, —S—, —S(═O)—, —SO₂—, or —NR⁷—. In some embodiments, Y is —C(═O)—.

In one embodiment of the PTCs of formula (I)-(VI), (A), (A-I), (B), and (C), Y is a bond, —CH₂—, —C(CH₃)H—, —O—, —S—, —NH—, —NCH₃—, or —N(COCH₃)—. In one embodiment, Y is —CH₂—, —C(CH₃)H—, —O—, —S—, —NH—, —NCH₃—, or —N(COCH₃)—. In some embodiments, Y is a bond, —CH₂—, —O—, or —NCH₃—. In some embodiments, Y is a bond, —CH₂—, —O—, or —NH—. In some embodiments of the PTCs of formula (I)-(VI) and (A)-(H-I), Y is —O—.

In one embodiment of the PTCs of formula (I)-(VI), (A), (B), and (C), V is a bond, —(CR^{8a}R^{9a})ₘ—, or —C(═O)—. In some embodiments, V is bond, or —(CR^{8a}R^{9a})ₘ—.

In one embodiment of the PTCs of formula (I)-(VI), (A), (A-I), (B), and (C), V is —(CR^{8a}R^{9a})ₘ—, wherein m is 1, 2, or 3. In one embodiment of the PTCs of formula (I)-(VI), (A), (B), and (C), V is —(CR^{8a}R^{9a})ₘ—, wherein R^{8a} and R^{8b} are each independently hydrogen, —OH, halogen, optionally substituted C₁-C₃ alkyl, optionally substituted C₂-C₃ alkenyl, optionally substituted C₂-C₃ alkynyl, C₁-C₃ alkoxy, —NR¹³R¹⁴, optionally substituted —(C₁-C₃ alkyl)-NR¹³R¹⁴, —NR¹⁴COR¹⁶, optionally substituted —(C₁-C₃ alkyl)-NR¹⁴COR¹⁶, —CONR¹⁴R¹⁵, or optionally substituted —(C₁-C₃ alkyl)-CONR¹⁴R¹⁵; or R^{8a} and R^{8b} taken together form an optionally substituted 3- to 6-membered carbocyclyl or heterocyclyl. In one embodiment, V is —(CR^{8a}R^{9a})ₘ—, wherein R^{8a} and R^{8b} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b}, on the same carbon atom or on a different carbon atom, taken together form an optionally substituted 3- to 6-membered carbocyclyl or heterocyclyl.

In one embodiment of the PTCs of formula (I)-(IIB) and (VA), V is —CH₂—, —CH₂CH₂—, —CH(CH₃)CH₂—, —CH₂CH(CH₃)—, or —CH₂CH₂CH₂—.

In one embodiment of the PTCs of formula (I)-(VI), (A), (B), and (C), V is —CH₂—, —CH₂CH₂—, or —CH₂CH₂CH₂—, each optionally substituted with one or more of —OH, halogen, or C₁-C₃ alkyl. In other embodiments, V is —CH₂—, —CH₂CH₂—, —CH₂CH(OH)CH₂— or —CH₂CH₂CH₂—. In some embodiments of the PTCs of formula (I)-(VI) and (A)-(H-I), V is —CH₂—, —CH₂CH₂—, or —CH₂CH₂CH₂—. In some embodiments of the PTCs of formula (I)-(VI) and (A)-(H-I), V is —CH₂— or —CH₂CH₂—.

In some embodiments of the PTCs of formula (I)-(VI) and (A)-(D), V is —CH₂— and L is halogen, —NH₂, or —CF₃; or V is —CH₂CH₂— and L is halogen or —NH₂.

In one embodiment of the PTCs of formula (I)-(VI), (A), (B), and (C), L is hydrogen, halogen, —CF₂H, —CF₃, —CN, —O(C₁-C₃ alkyl), —NR¹¹R¹², or —CONR¹¹R¹². In one embodiment, L is hydrogen, halogen, —CF₂H, —CF₃, —CN, —O(C₁-C₃ alkyl), —NH₂, —NH(C₁-C₃ alkyl), —N(C₁-C₃ alkyl)₂, —CONH₂, —CONH(C₁-C₃ alkyl), or —CON(C₁-C₃ alkyl)₂. In some embodiments, L is hydrogen, halogen, —CF₃, or —NH₂.

In some embodiments of the PTCs of formula (IC) and (IIIA), L is halogen, —CCl₃, —CCl₂, —CF₃, or —NH₂. In some embodiments of the PTCs of formula (I)-(VI) and (A)-(H-I), L is halogen, —CF₃, or —NH₂. In one embodiment, L is hydrogen or halogen. In one embodiment, L is halogen. In other embodiments, L is Cl, or Br. In one embodiment, L is Cl.

In one embodiment of the PTCs of formula (I)-(VI), (A), (B), and (C), W is a bond. In one embodiment, W is —(CR^{8a}R^{9a})ₘ—, —C(═O)—, —N(R⁷)CO—, —CONR⁷—, or —NSO₂R⁷—. In one embodiment, W is —(CR^{8a}R^{9a})ₘ—, wherein m is 1, 2, or 3. In some embodiments, W is a bond, —CH₂—, —C(CH₃)H—, —C(═O)—, —N(R⁷)CO—, or —CONR⁷—, wherein R⁷ is H or C₁-C₆ alkyl. In some embodiments, W is a bond, —CH₂—, —C(CH₃)H—, —C(═O)—, —N(R⁷)CO—, or —CONR⁷—, wherein R⁷ is H or C₁-C₃ alkyl. In one embodiment, W is a bond, —CH₂—, —C(CH₃)H—, —C(═O)—, —NHCO—, —N(C₁-C₃ alkyl)CO—, —CONH—, or —CON(C₁-C₃ alkyl)-. In one embodiment of the PTCs of formula (I)-(VI) and (A)-(E-VI), W is a bond, —CH₂—, or —C(CH₃)H—. In one embodiment of the PTCs of formula (I)-(VI) and (A)-(H-I), W is a —CH₂— or —C(CH₃)H—.

In one embodiment of the PTCs of formula (I)-(VI), (A), (B), and (C), —Y—W— is a bond, —OCH₂—, —OCH₂CH₂—, —OCH(CH₃)—, —NH—, —NHCH₂—, —NHC(═O)—, or —C(═O)NH—. In one embodiment of the PTCs of formula (I)-(VI) and (A)-(H-I), —Y—W— is —OCH₂—, —OCH₂CH₂—, or —OCH(CH₃)—.

In one embodiment of the PTCs of formula (I)-(VI), (A), (B), and (C),
Z is a bond, —CH₂—, —C(CH₃)H—, —O—, —S—, —NH—, —NCH₃—, or —N(COCH₃)—; and
V is —CH₂—, —CH₂CH₂—, or —CH₂CH₂CH₂—.

In one embodiment of the PTCs of formula (I)-(VI), (A), (B), and (C),
Z is a bond, —CH₂—, —C(CH₃)H—, —O—, —S—, —NH—, —NCH₃—, or —N(COCH₃)—;
V is —CH₂—, —CH₂CH₂—, or —CH₂CH₂CH₂—; and
L is halogen, —NH₂, or —CF₃.

In one embodiment of the PTCs of formula (I)-(VI), (A), (B), and (C), —Z—V-L is —Z—CH₂CH₂Cl, —Z—CH₂CH₂CH₂Cl, —Z—CH₂CH₂NH₂, or —Z—CH₂CH₂CH₂NH₂, wherein Z is a bond, —O—, —NH—, or —N(COCH₃)—. In one embodiment, —Z—V-L is —OCH₃.

In one embodiment of the PTCs of formula (I)-(VI) and (A)-(H-I), —Z—V-L is —O—CH₂CH₂Cl or —O—CH₂CH₂CH₂Cl.

In one embodiment of the PTCs of formula (I)-(VI), (A), (B), and (C), —V-L is —CH₂CH₂Cl, —CH₂CH₂CH₂Cl, —CH₂CH₂NH₂, or —CH₂CH₂CH₂NH₂. In one embodiment, —V-L is —CH₃.

In one embodiment of the PTCs of formula (I)-(VI), (A), (B), and (C), X is a bond, —(CR⁵R⁶)ₜ—, —O—, —C(═O)—, —S—, —S(═O)—, —SO₂—, or —NR⁷—. In one embodiment, X is a bond, —(CR⁵R⁶)ₜ—, —O—, —C(═O)—, —S—, —S(═O)—, —SO₂—, or —NR⁷—, wherein R⁷ is H or C₁-C₆ alkyl. In some embodiments, X is a bond, —(CR⁵R⁶)ₜ—, or —NR⁷—. In some embodiments, X is a bond or —(CR⁵R⁶)ₜ—. In some embodiments, X is a bond, —CH₂—, —C(CH₃)H—, —C(CH₃)₂—, —CH₂CH₂—, —NH—, or —N(C₁-C₆ alkyl)-. In some embodiments, X is a bond, —CH₂—, —C(CH₃)H—, —C(CH₃)₂—, —CH₂CH₂—, —NH—, —N(CH₃)—, —N(CH₂CH₃)—, —N(iPr)-, or —N(tBu)-. In some embodiments, X is a bond, —CH₂—, —C(CH₃)H—, —C(CH₃)₂—, or —CH₂CH₂—.

In one embodiment of the PTCs of formula (I)-(VI) and (A)-(H-I), X is —CH₂—, —C(CH₃)H—, or —C(CH₃)₂—. In one embodiment, X is —C(CH₃)₂—.

In one embodiment of the PTCs of formula (I)-(VI), (A), (B), and (C), R¹ and R² are each independently halogen, —CN, —CF₃, —OH, C₁-C₃ alkyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₃ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂R¹⁶, —(C₁-C₃ alkyl)-SO₂R¹⁶, 3- to 7-membered carbocyclyl, 3- to 7-membered heterocyclyl, phenyl, or 5- to 6-membered heteroaryl.

In one embodiment of the PTCs of formula (I)-(VI), (A), (A-I), (B), and (C), R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶. In one embodiment, R¹ and R² are each independently hydrogen, halogen, —CN, —CF₃, —OH, optionally substituted C₁-C₃ alkyl, C₁-C₃ alkoxy, optionally substituted —(C₁-C₃ alkyl)-(C₁-C₃ alkoxy), optionally substituted —(C₁-C₃ alkyl)-OH, —NR¹³R¹⁴, —(C₁-C₃ alkyl)-NR¹³R¹⁴, —NR¹⁴SO₂R¹⁶, optionally substituted —(C₁-C₃ alkyl)NR¹⁴SO₂R¹⁶, —NR¹⁴COR¹⁶, optionally substituted —(C₁-C₃ alkyl)-NR¹⁴COR¹⁶, —CONR¹⁴R¹⁵, optionally substituted —(C₁-C₃ alkyl)-CONR¹⁴R¹⁵, —SO₂NR¹⁴R¹⁵, optionally substituted —(C₁-C₃ alkyl)-SO₂NR¹⁴R¹⁵, —SO₂R¹⁶, or optionally substituted —(C₁-C₃ alkyl)-SO₂R¹⁶. In one embodiment, R¹ and R² are each independently hydrogen, halogen, —CN, —CF₃, —OH, C₁-C₃ alkyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₃ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂R¹⁶, or —(C₁-C₃ alkyl)-SO₂R¹⁶.

In one embodiment of the PTCs of formula (I)-(VI), (A), (A-I), (B), and (C), R¹ and R² are each independently hydrogen, halogen, —CN, —CF₃, —OH, C₁-C₃ alkyl, or —CONR¹⁴R¹⁵. In some embodiments, R¹ and R² are each independently hydrogen, halogen, —CN, —CF₃, —OH, methyl, methoxy, or —CONH₂. In one embodiment, R¹ and R² are each independently hydrogen, Cl, —CN, —CF₃, —OH, methyl, methoxy, or —CONH₂. In one embodiment, R¹ and R² are each independently hydrogen, halogen, —CN, —CF₃, —OH, or methyl. In one embodiment, R¹ and R² are each independently Cl, —CN, —CF₃, —OH, methyl, methoxy, or —CONH₂. In one embodiment of the PTCs of formula (I)-(VI), R¹ and R² are each independently halogen, —CN, —CF₃, —OH, or methyl.

In one embodiment of the PTCs of formula (I)-(VI) and (A)-(E-I), R¹ and R² are each halogen, methyl, —CF₃, or —CN. In one embodiment of the PTCs of formula (I)-(VI) and (A)-(F), R¹ and R² are each halogen or —CN. In one embodiment of the PTCs of formula (I)-(VI) and (A)-(H-I), at least one of R¹ and R² is Cl or —CN. In one embodiment of the PTCs of formula (I)-(VI) and (A)-(H-I), at least two of R¹ and R² are each independently Cl or —CN. In one embodiment of the PTCs of formula (I)-(VI) and (A)-(H-I), R¹ and R² are each Cl or —CN.

In one embodiment of the PTCs of formula (I)-(VI), (A), (B), and (C), R¹ and R² are each independently optionally substituted carbocyclyl, optionally substituted heterocyclyl, optionally substituted aryl, or optionally substituted heteroaryl. In one embodiment, R¹ and R² are each independently 3- to 7-membered carbocyclyl, 3- to 7-membered heterocyclyl, phenyl, or 5- to 6-membered heteroaryl.

In one embodiment of the PTCs of formula (I)-(VI), (A), (A-I), (B), and (C), R¹ have one of the connectivity as shown below with respect to X and Y:

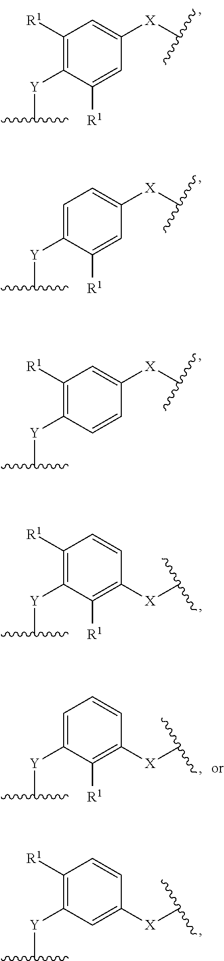

In one embodiment of the PTCs of formula (I)-(VI), (A), (A-I), (B), and (C), R² have one of the connectivity as shown below with respect to X and Z:

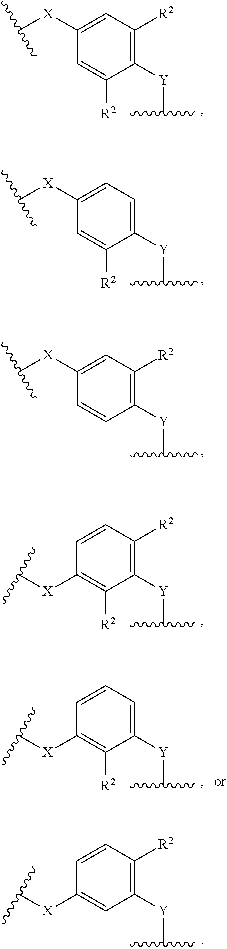

In one embodiment of the PTCs of formula (I)-(VI), (A), (A-I), (B), and (C), n1 is 0, 1, or 2. In some embodiments, n1 is 0 or 1. In other embodiments, n1 is 0. In some embodiments, n1 is 1. In one embodiment, the sum of n1 and n2 is 0, 1, 2, 3, or 4. In some embodiments, the sum of n1 and n2 is 1, 2, 3, or 4. In one embodiment, the sum of n1 and n2 is 2.

In one embodiment of the PTCs of formula (I)-(VI), (A), (A-I), (B), and (C), n2 is 0, 1, or 2. In some embodiments, n2 is 1 or 2. In other embodiments, n2 is 0. In some embodiments, n2 is 1. In some embodiments, n2 is 2.

In some embodiments of the PTCs of formula (I)-(VI), (A), (A-I), (B), and (C), n3 is 1, 2, 3, 4, or 5. In some embodiments, n3 is 1, 2, 3, or 4. In one embodiment, n3 is 1, 2, or 3. In one embodiment, n3 is 1 or 2.

In one embodiment of the PTCs of formula (I)-(VI), (A), (A-I), (B), and (C), R³ is selected from hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl. In some embodiments, R³ is selected from hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶. In another embodiment, R³ is hydrogen, halogen, oxo, ═S, ═NR¹⁶, —CN, —CF₃, —OH, —SR¹⁶, optionally substituted C₁-C₃ alkyl, optionally substituted C₂-C₃ alkenyl, optionally substituted C₂-C₃ alkynyl, optionally substituted C₁-C₃ alkoxy, optionally substituted —(C₁-C₃ alkyl)-(C₁-C₃ alkoxy), optionally substituted —(C₁-C₃ alkyl)-OH, —NR¹³R¹⁴, optionally substituted —(C₁-C₃ alkyl)-NR¹³R¹⁴, —NR¹⁴SO₂R¹⁶, optionally substituted —(C₁-C₃ alkyl)NR¹⁴SO₂R¹⁶, —NR¹⁴COR¹⁶, optionally substituted —(C₁-C₃ alkyl)-NR¹⁴COR¹⁶, —CONR¹⁴R¹⁵, optionally substituted —(C₁-C₃ alkyl)-CONR¹⁴R¹⁵, —SO₂NR¹⁴R¹⁵, optionally substituted —(C₁-C₃ alkyl)-SO₂NR¹⁴R¹⁵, optionally substituted —SO₂R¹⁶, or optionally substituted —(C₁-C₃ alkyl)-SO₂R¹⁶.

In one embodiment of the PTCs of formula (I)-(VI), (A), (A-I), (B), and (C), R³ is selected from —NR¹⁴SO₂R¹⁶, optionally substituted —(C₁-C₆ alkyl)NR¹⁴SO₂R¹⁶, or optionally substituted —SO₂R¹⁶; wherein R¹⁶ is hydrogen, optionally substituted C₁-C₃ alkyl, optionally substituted C₂-C₃ alkenyl, optionally substituted C₂-C₃ alkynyl, C₃-C₆ cycloalkyl, or phenyl. In one embodiment, R³ is selected from —NR¹⁴SO₂R¹⁶, —(C₁-C₆ alkyl)NR¹⁴SO₂R¹⁶, or —SO₂R¹⁶; wherein R¹⁶ is hydrogen, C₁-C₃ alkyl, —(C₁-C₃ alkyl)-NH₂, C₃-C₆ cycloalkyl, or phenyl.

In one embodiment of the PTCs of formula (I)-(VI), (A), (A-I), (B), and (C), R³ is selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). In some embodiments, R³ is selected from hydrogen, F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In some embodiments, R³ is selected from hydrogen, F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In one embodiment, R³ is selected from hydrogen, F, Cl, Br, I, oxo, ═S, ═NR¹⁶, —CN, —CF₃, —OH, methyl, —SCH₃, —SO₂CH₃, —NHSO₂CH₃, —NHSO₂CH₂CH₃, —CH₂NHSO₂CH₃, —SO₂NH₂, —CONH₂, or —NHCOCH₃. In one embodiment, R³ is selected from hydrogen, F, Cl, Br, I, oxo, ═S, ═NR¹⁶, —CN, —CF₃, —OH, methyl, —SCH₃, —SO₂CH₃, —NHSO₂CH₃, —CH₂NHSO₂CH₃, —SO₂NH₂, —CONH₂, or —NHCOCH₃. In some embodiments, R³ is selected from F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In some embodiments, R³ is selected from F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In one embodiment, R³ is selected from F, Cl, Br, I, oxo, ═S, ═NR¹⁶, —CN, —CF₃, —OH, methyl, —SCH₃, —SO₂CH₃, —NHSO₂CH₃, —CH₂NHSO₂CH₃, —SO₂NH₂, —CONH₂, or —NHCOCH₃. In another embodiment, R³ is —SO₂CH₃, —NHSO₂CH₃, —CH₂NHSO₂CH₃, —SO₂NH₂, —CONH₂, or —NHCOCH₃. In one embodiment, R³ is oxo, ═S, ═NR¹⁶, C₁-C₃ alkyl, —SO₂(C₁-C₃ alkyl), or —NHSO₂(C₁-C₃ alkyl). In one embodiment, at least one of R³ is oxo, ═S, or ═NR¹⁶. In one embodiment, at least one of R³ is oxo, ═S, or ═NR¹⁶, wherein R¹⁶ is H or C₁-C₃ alkyl.

In one embodiment of the PTCs of formula (I)-(VI) and (A)-(E-VII), R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl). In one embodiment of the PTCs of formula (I)-(VI) and (A)-(E-VII), R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂CH₃, —NHSO₂CF₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the PTCs of formula (I)-(VI), (A), (A-I), (B), and (C), R³ on a sp³ carbon is each selected from hydrogen, halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). When R³ on a sp³ carbon is oxo, ═S, or ═NR¹⁶, the carbon becomes sp².

In one embodiment of the PTCs of formula (I)-(VI), (A), (A-I), (B), and (C), R³ on a sp² carbon is each selected from hydrogen, halogen, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl).

In one embodiment of the PTCs of formula (I)-(VI), (A), (A-I), (B), and (C), R³ on a nitrogen atom is each selected from C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —(C₁-C₃ alkyl)-NR¹³R¹⁴, —(C₁-C₃ alkyl)NR¹⁴SO₂R¹⁶, —(C₁-C₆ alkyl)-NR¹⁴COR¹⁶, —CONR¹⁴R¹⁵, —(C₁-C₃ alkyl)-CONR¹⁴R¹⁵, —(C₁-C₃ alkyl)-SO₂NR¹⁴R¹⁵, or —(C₁-C₆ alkyl)-SO₂(C₁-C₃ alkyl).

In one embodiment of the PTCs of formula (I)-(VI) and (A)-(G-II), at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl). In one embodiment, at least one R³ is selected from —NHSO₂CH₃, —NHSO₂CH₂CH₃, or —SO₂CH₃ and the other R³, if present, is selected from —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the PTCs of formula (I)-(VI) and (A)-(E-II), R³ is not hydrogen.

In one embodiment of the PTCs of formula (I)-(VI) and (A)-(G-II), at least one R³ is —SO₂CH₃, —NHSO₂CH₃, —NCH₃SO₂CH₃, —NHSO₂CH₂CH₃, or —N(CH₃)SO₂CH₂CH₃. In one embodiment of the PTCs of formula (I)-(VI) and (A)-(G-II), at least one R³ is —SO₂CH₃, —NHSO₂CH₃, or —NCH₃SO₂CH₃.

In one embodiment the compounds of formula (I), (IA), (IB), or (IC), R³ is heterocyclyl. In one embodiment, R³ is heterocyclyl selected from

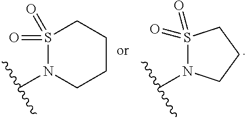 [SMILES: *N1CCCCS1(=O)=O ; *N1CCCS1(=O)=O]

In one embodiment of the PTCs of formula (IB), (IC), (IIA), or (IIB), R³ is —NR¹⁴SO₂R¹⁶, wherein R¹⁴ and R¹⁶ together form a 5 or 6 membered ring including the nitrogen and sulfur atoms.

In one embodiment of the PTCs of formula (I), (IA), (IB), or (IC), R³ is —NR¹⁴SO₂R¹⁶, wherein R¹⁶ is optionally substituted C₁-C₆ alkyl. In one embodiment, R³ is —NR¹⁴SO₂R¹⁶, wherein R¹⁶ is C₁-C₆ alkyl optionally substituted with one or more groups selected from halogen, —CN, —CF₃, —OH, C₁-C₃ alkyl, C₁-C₃ alkoxy, —NH₂, —NH(C₁-C₃ alkyl), —N(C₁-C₃ alkyl)₂, —SCH₃. In one embodiment, R³ is —NR¹⁴SO₂R¹⁶, wherein R¹⁶ is C₁-C₃ alkyl substituted with —NH₂.

In one embodiment of the PTCs of formula (I)-(VI), (A), (B), and (C), R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl. In some embodiments, R⁵ and R⁶ are each independently hydrogen, halogen, —OH, optionally substituted C₁-C₆ alkyl, optionally substituted C₂-C₆ alkenyl, optionally substituted C₂-C₆ alkynyl. In one embodiment, R⁵ and R⁶ are hydrogen, halogen, —OH, or C₁-C₃ alkyl. In one embodiment, R⁵ and R⁶ are each independently hydrogen, F, —OH, or C₁-C₃ alkyl. In one embodiment, R⁵ and R⁶ are each independently, hydrogen, F, —OH, or methyl. In one embodiment, R⁵ and R⁶ are each H. In one embodiment, R⁵ and R⁶ are each methyl. In one embodiment of the PTCs of formula (I)-(VI) and (A)-(H-I), R⁵ and R⁶ are each H or methyl.

In one embodiment of the PTCs of formula (I)-(VI), (A), (B), and (C), R⁷ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R⁷ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R⁷ is hydrogen, C₁-C₆ alkyl, C₂-C₆ alkenyl, C₂-C₆ alkynyl, carbocyclyl, heterocyclyl, aryl, or heteroaryl. In some embodiments, R⁷ is hydrogen, C₁-C₃ alkyl, C₂-C₃ alkenyl, C₂-C₃ alkynyl, carbocyclyl, heterocyclyl, aryl, or heteroaryl. In some embodiments, R⁷ is hydrogen, C₁-C₆ alkyl, C₂-C₆ alkenyl, or C₂-C₆ alkynyl. In some embodiments, R⁷ is hydrogen, C₁-C₃ alkyl, C₂-C₃ alkenyl, or C₂-C₃ alkynyl. In some embodiments, R⁷ is hydrogen or C₁-C₆ alkyl. In some embodiments, R⁷ is hydrogen or C₁-C₄ alkyl. In some embodiments of the PTCs of formula (I)-(VI) and (A)-(H-I), R⁷ is hydrogen or C₁-C₃ alkyl.

In one embodiment of the PTCs of formula (I)-(VI), (A), (B), and (C), R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl, or R^{8a} and R^{9a} taken together form an optionally substituted carbocyclyl or optionally substituted heterocyclyl. In some embodiments, R^{8a} and R^{8b} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl. In one embodiment, R^{8a} and R^{9a} are each independently hydrogen, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵. In one embodiment of the PTCs of formula (IB), (IC), (III), or (IIIA), R^{8a} and R^{9a} are not —OH. In one embodiment, R^{8a} and R^{9a} are not —OH.

In one embodiment of the PTCs of formula (I)-(VI), (A), (B), and (C), R⁷ and R^{8a} taken together form an optionally substituted heterocyclyl. In one embodiment, R⁷ and R^{8a} taken together form an optionally substituted 3- to 7-membered heterocycle.

In one embodiment of the PTCs of formula (I)-(VI), (A), (B), and (C), R⁸ and R⁹ are each independently hydrogen, halogen, or C₁-C₃ alkyl.

In one embodiment of the PTCs of formula (I)-(IIB), R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R¹⁰ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R¹⁰ is hydrogen, C₁-C₃ alkyl, C₂-C₃ alkenyl, C₂-C₃ alkynyl, carbocyclyl, heterocyclyl, aryl, or heteroaryl. In some embodiments, R¹⁰ is hydrogen, C₁-C₃ alkyl, C₂-C₃ alkenyl, C₂-C₃ alkynyl. In some embodiments, R¹⁰ is hydrogen or C₁-C₃ alkyl.

In one embodiment of the PTCs of formula (I)-(IIB), R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹¹ and R¹² are each independently hydrogen, optionally substituted C₁-C₆ alkyl, optionally substituted C₂-C₆ alkenyl, or optionally substituted C₂-C₆ alkynyl. In some embodiments. R¹¹ and R¹² are each independently hydrogen, C₁-C₆ alkyl, C₂-C₆ alkenyl, or C₂-C₆ alkynyl. In some embodiments. R¹¹ and R¹² are each independently hydrogen or C₁-C₃ alkyl.

In one embodiment of the PTCs of formula (I)-(IIB), R¹¹ and R¹² taken together form an optionally substituted heterocyclyl. In one embodiment, R¹¹ and R¹² taken together form an optionally substituted 3- to 7-membered heterocyclyl. In other embodiments, R¹¹ and R¹² taken together form 3- to 7-membered heterocyclyl.

In one embodiment of the PTCs of formula (I)-(VI), (A), (B), and (C), R¹³ and R¹⁴ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹³ and R¹⁴ are each independently hydrogen, optionally substituted C₁-C₆ alkyl, optionally substituted C₂-C₆ alkenyl, or optionally substituted C₂-C₆ alkynyl. In some embodiments R¹³ and R¹⁴ are each independently hydrogen, C₁-C₆ alkyl, C₂-C₆ alkenyl, or C₂-C₆ alkynyl. In some embodiments R¹³ and R¹⁴ are each independently hydrogen or C₁-C₃ alkyl.

In one embodiment of the PTCs of formula (I)-(VI), (A), (B), and (C), R¹⁵ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁵ is hydrogen, optionally substituted C₁-C₆ alkyl, optionally substituted C₂-C₆ alkenyl, or optionally substituted C₂-C₆ alkynyl. In some embodiments, R¹⁵ is hydrogen, C₁-C₆ alkyl, C₂-C₆ alkenyl, or C₂-C₆ alkynyl. In some embodiments, R¹⁵ is hydrogen or C₁-C₃ alkyl.

In one embodiment of the PTCs of formula (I)-(VI), (A), (B), and (C), R¹⁴ and R¹⁵ taken together form an optionally substituted heterocyclyl. In one embodiment, R¹⁴ and R¹⁵ taken together form an optionally substituted 3- to 7-membered heterocyclyl. In other embodiments, R¹⁴ and R¹⁵ taken together form 3- to 7-membered heterocyclyl.

In one embodiment of the PTCs of formula (I)-(VI), (A), (B), and (C), R¹⁶ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R¹⁶ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R¹⁶ is hydrogen, C₁-C₃ alkyl, C₂-C₃ alkenyl, C₂-C₃ alkynyl, carbocyclyl, heterocyclyl, aryl, or heteroaryl. In some embodiments, R¹⁶ is hydrogen, C₁-C₃ alkyl, C₂-C₃ alkenyl, or C₂-C₃ alkynyl. In some embodiments, R¹⁶ is hydrogen or C₁-C₃ alkyl.

In one embodiment of the PTCs of formula (I)-(IIIA), m is 1 or 2.

In one embodiment of the PTCs of formula (I)-(VI) and (A)-(F), t is 1 or 2. In one embodiment of the PTCs of formula (I)-(VI) and (A)-(H-I), t is 1.

In one embodiment of the PTCs of formula (I)-(VI), (A), (B), and (C), optional substituent is selected from halogen, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). In another embodiment, the optional substituent is selected from halogen, —CN, —CF₃, —OH, C₁-C₃ alkyl, C₁-C₃ alkoxy, —NH₂, —SCH₃, —SO₂CH₃, —NHSO₂CH₃, —CH₂NHSO₂CH₃, —SO₂NH₂, —CONH₂, or —NHCOCH₃.

In one embodiment of the PTCs of formula (I)-(VI), A and B are each monocyclic ring.

In one embodiment of the PTCs of formula (I)-(VI), B is phenyl, pyridyl, or pyrimidyl.

In one embodiment of the PTCs of formula (I)-(IIIA), Z and V are not both a bond.

In one embodiment of the PTCs of formula (I)-(VI), (A)-(C), Y and W are not both a bond.

In one embodiment of the PTCs of formula (I)-(VI), C is a 4- to 10-membered ring.

In one embodiment of the PTCs of formula (D)-(H-I), X is a bond, —CH₂—, —C(CH₃)H—, —C(CH₃)₂—, or —CH₂CH₂—. In one embodiment, X is —CH₂—, —C(CH₃)H—, or —C(CH₃)₂—. In some embodiments, X is —C(CH₃)₂—.

In one embodiment of the PTCs of formula (D)-(H), X is —NR⁷—. In one embodiment, X is —NH—, —N(CH₃)—, —N(CH₂CH₃)—, —N(iPr)-, or —N(tBu)-.

In one embodiment of the PTCs of formula (D)-(H-I), Y is —O—. In one embodiment of the PTCs of formula (D)-(H-I), Z is —O—. In one embodiment of the PTCs of formula (D)-(H-I), Y and Z are both —O—.

In one embodiment of the PTCs of formula (D)-(H-I), —V-L is CH₂CH₂Cl, —CH₂CH₂CH₂Cl, or —CH₃. In some embodiments, —V-L is CH₂CH₂Cl or —CH₂CH₂CH₂Cl.

In one embodiment of the PTCs of formula (D)-(H-I), n1 is 0.

In one embodiment of the PTCs of formula (D)-(H-I), n2 is 0, 1, or 2. In some embodiments, n2 is 2. In some embodiments, n2 is 2 and R² are each ortho to Z. In other embodiments, n2 is 2 and R² are each ortho to Z, wherein R² is halogen or —CN.

In one embodiment of the PTCs of formula (I)-(VI) and (A)-(H-I), the compound can be a stereoisomer. For example, if X is —(CR⁵R⁶)— and R⁵ and R⁶ are different, the carbon attached to R⁵ and R⁶ can be in an S configuration or an R configuration.

In some embodiments of the PTCs of formula (I)-(VI) and (A)-(H-I), a hydrogen atom can be replaced with a deuterium atom.

In one embodiment of the PTCs of formula (I)-(VA), (A), (A-I), or (D)-(H-I), the PTC is selected from Table A below, or a pharmaceutically acceptable salt, tautomer, stereoisomer or prodrug thereof. In one embodiment of the PTC of formula (I)-(VA), (A), or (D)-(H-I), the PTC is selected from PTC A3, A5, A7, A13, A17, A18, A22, A23, A24, A25, A28, A30, A31, A32, A34, A35, A38, A40, A41, A42, A45, A49, A52, A53, A54, A56, A57, A58, A62, A63, A64, A65, A68, A73, A74, A75, or A76, or a pharmaceutically acceptable salt, tautomer, stereoisomer or prodrug thereof. In one embodiment of the PTC of formula (I)-(VA), (A), (A-I), or (D)-(H-I), the PTC is selected from PTC A1, A2, A4, A6, A8, A9, A10, A11, A12, A14, A15, A16, A19, A20, A21, A26, A27, A29, A33, A36, A37, A39, A43, A44, A46, A47, A48, A50, A51, A55, A59, A60, A61, A66, A67, A69, A70, A71, A72, A77, A78, A79, A80, A81, A82, A83, A84, A85, A86, A87, A88, A89, A90, A91, A92, A93, A94, A95, A96, or A97, or a pharmaceutically acceptable salt, tautomer, stereoisomer or prodrug thereof. In one embodiment of the PTC of formula (I)-(VA), (A), (A-I), or (D)-(H-I), the PTC is selected from PTCs A98-A186, or a pharmaceutically acceptable salt, tautomer, stereoisomer or prodrug thereof. In one embodiment of the PTC of formula (I)-(VA), (A), (A-I), or (D)-(H-I), the PTC is selected from PTCs A187-A211, or a pharmaceutically acceptable salt, tautomer, stereoisomer or prodrug thereof. In one embodiment of the PTC of formula (I)-(VA), (A), (A-I), or (D)-(H-I), the PTC is selected from PTCs A1-A211, or a pharmaceutically acceptable salt, tautomer, stereoisomer or prodrug thereof. In one embodiment of the PTC of formula (I)-(VA), (A), (A-I), or (D)-(H-I), the PTC is selected from PTCs A212-A234, or a pharmaceutically acceptable salt, tautomer, stereoisomer or prodrug thereof. In one embodiment of the PTC of formula (I)-(VA), (A), (A-I), or (D)-(H-I), the PTC is selected from PTCs A1-A234, or a pharmaceutically acceptable salt, tautomer, stereoisomer or prodrug thereof. In one embodiment of the PTC of formula (I)-(VA), (A), (A-I), or (D)-(H-I), the PTC is selected from PTCs A1-A96, A98-A116, A118-A159, A161-A175, and A177-A234, or a pharmaceutically acceptable salt, tautomer, stereoisomer or prodrug thereof.

In one embodiment of the PTC of formula (I)-(VA), (A), (A-I), or (D)-(H-I), the PTC is selected from A13, A57, A74, A93, A109, A112, A122, A126, A131, A134, A136, A137, A164, A168, A169, A170, A171, A172, A184, A185, A195, and/or A204, or a pharmaceutically acceptable salt, tautomer, stereoisomer or prodrug thereof.

In one embodiment, PTC in formula Q is a compound of formula (I)-(VA), (A), (A-I), or (D)-(H-I), minus any functional group that was involved in making the PTC-LI bond.

TABLE A
PTCs

| PTC ID | Structure |
|---|---|
| A1 | 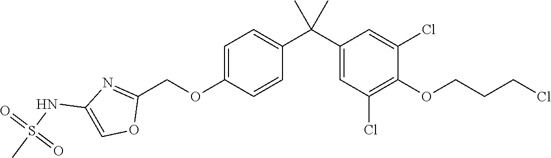 [SMILES: CC(C)(c1ccc(OCc2nc(NS(C)(=O)=O)co2)cc1)c1cc(Cl)c(OCCCCl)c(Cl)c1] |
| A2 | 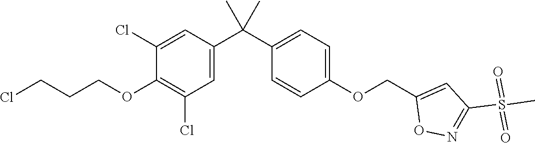 [SMILES: CC(C)(c1ccc(OCc2cc(S(C)(=O)=O)no2)cc1)c1cc(Cl)c(OCCCCl)c(Cl)c1] |
| A3 | 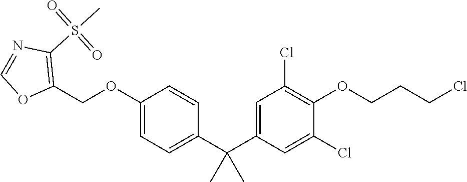 [SMILES: CC(C)(c1ccc(OCc2ocnc2S(C)(=O)=O)cc1)c1cc(Cl)c(OCCCCl)c(Cl)c1] |
| A4 | 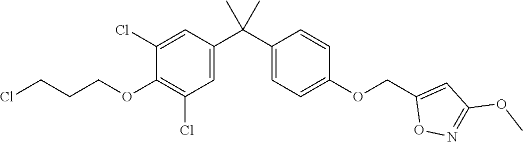 [SMILES: COc1cc(COc2ccc(C(C)(C)c3cc(Cl)c(OCCCCl)c(Cl)c3)cc2)on1] |
| A5 | 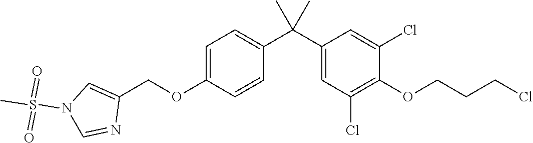 [SMILES: CC(C)(c1ccc(OCc2cn(S(C)(=O)=O)cn2)cc1)c1cc(Cl)c(OCCCCl)c(Cl)c1] |
| A6 | 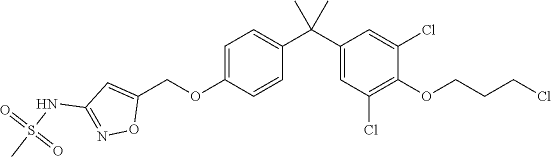 [SMILES: CC(C)(c1ccc(OCc2cc(NS(C)(=O)=O)no2)cc1)c1cc(Cl)c(OCCCCl)c(Cl)c1] |
| A7 | 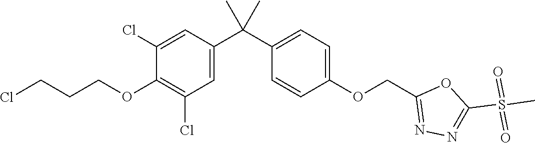 [SMILES: CC(C)(c1ccc(OCc2nnc(S(C)(=O)=O)o2)cc1)c1cc(Cl)c(OCCCCl)c(Cl)c1] |
| A8 | 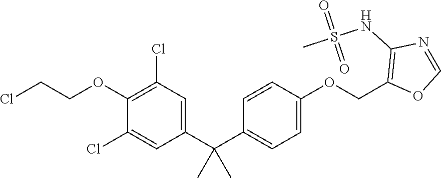 [SMILES: CC(C)(c1ccc(OCc2ocnc2NS(C)(=O)=O)cc1)c1cc(Cl)c(OCCCl)c(Cl)c1] |
| A9 | 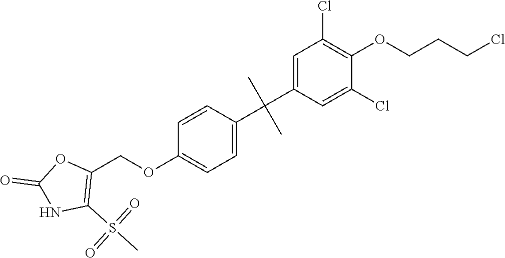 [SMILES: CC(C)(c1ccc(OCc2oc(=O)[nH]c2S(C)(=O)=O)cc1)c1cc(Cl)c(OCCCCl)c(Cl)c1] |
| A10 | 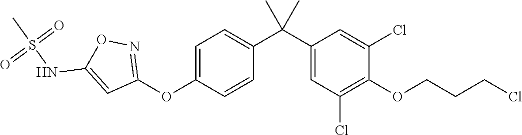 [SMILES: CC(C)(c1ccc(Oc2cc(NS(C)(=O)=O)on2)cc1)c1cc(Cl)c(OCCCCl)c(Cl)c1] |
| A11 | 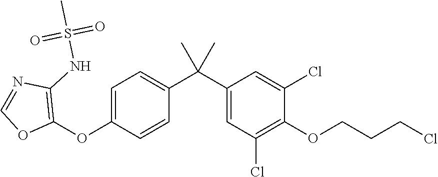 [SMILES: CC(C)(c1ccc(Oc2ocnc2NS(C)(=O)=O)cc1)c1cc(Cl)c(OCCCCl)c(Cl)c1] |
| A12 | 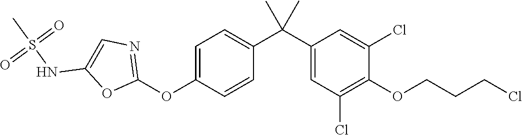 [SMILES: CC(C)(c1ccc(Oc2ncc(NS(C)(=O)=O)o2)cc1)c1cc(Cl)c(OCCCCl)c(Cl)c1] |
| A13 | 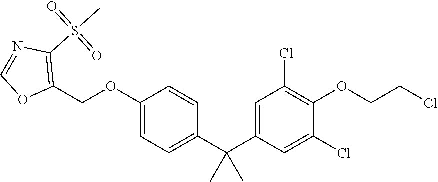 [SMILES: CC(C)(c1ccc(OCc2ocnc2S(C)(=O)=O)cc1)c1cc(Cl)c(OCCCl)c(Cl)c1] |
| A14 | 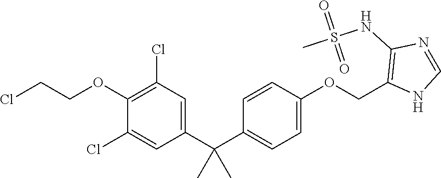 [SMILES: CC(C)(c1ccc(OCc2[nH]cnc2NS(C)(=O)=O)cc1)c1cc(Cl)c(OCCCl)c(Cl)c1] |
| A15 | 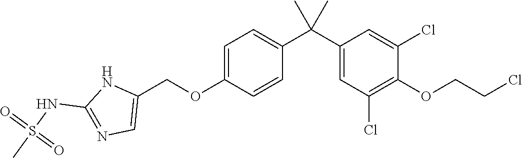 [SMILES: CC(C)(c1ccc(OCc2cnc(NS(C)(=O)=O)[nH]2)cc1)c1cc(Cl)c(OCCCl)c(Cl)c1] |
| A16 | 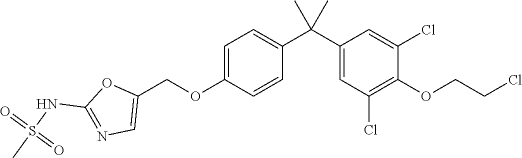 [SMILES: CC(C)(c1ccc(OCc2cnc(NS(C)(=O)=O)o2)cc1)c1cc(Cl)c(OCCCl)c(Cl)c1] |
| A17 | 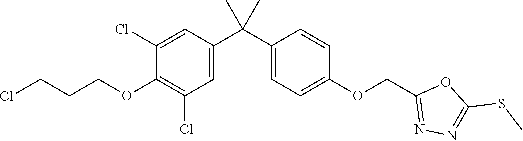 [SMILES: CSc1nnc(COc2ccc(C(C)(C)c3cc(Cl)c(OCCCCl)c(Cl)c3)cc2)o1] |
| A18 | 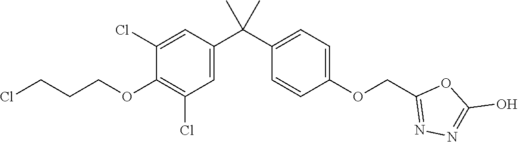 [SMILES: CC(C)(c1ccc(OCc2nnc(O)o2)cc1)c1cc(Cl)c(OCCCCl)c(Cl)c1] |
| A19 | 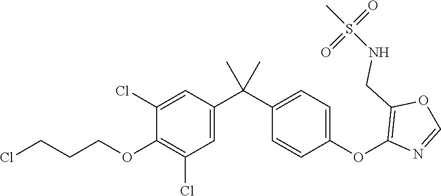 [SMILES: CC(C)(c1ccc(Oc2ncoc2CNS(C)(=O)=O)cc1)c1cc(Cl)c(OCCCCl)c(Cl)c1] |
| A20 | 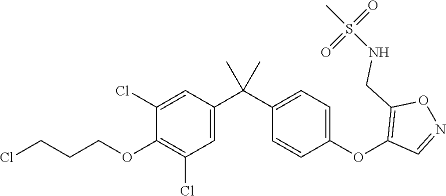 [SMILES: CC(C)(c1ccc(Oc2cnoc2CNS(C)(=O)=O)cc1)c1cc(Cl)c(OCCCCl)c(Cl)c1] |
| A21 | 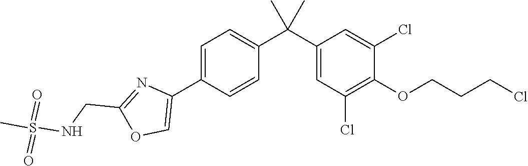 [SMILES: CC(C)(c1ccc(-c2coc(CNS(C)(=O)=O)n2)cc1)c1cc(Cl)c(OCCCCl)c(Cl)c1] |
| A22 | 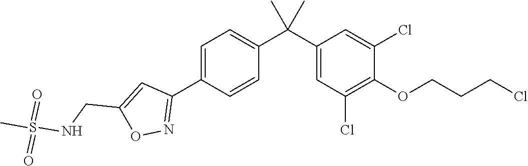 [SMILES: CC(C)(c1ccc(-c2cc(CNS(C)(=O)=O)on2)cc1)c1cc(Cl)c(OCCCCl)c(Cl)c1] |
| A23 | 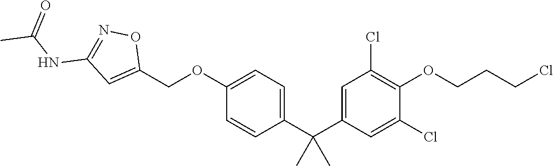 [SMILES: CC(=O)Nc1cc(COc2ccc(C(C)(C)c3cc(Cl)c(OCCCCl)c(Cl)c3)cc2)on1] |
| A24 | 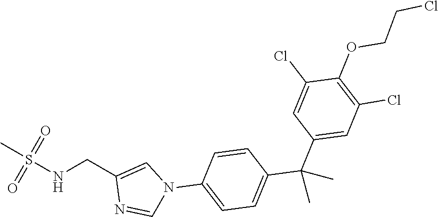 [SMILES: CC(C)(c1ccc(-n2cnc(CNS(C)(=O)=O)c2)cc1)c1cc(Cl)c(OCCCl)c(Cl)c1] |
| A25 | 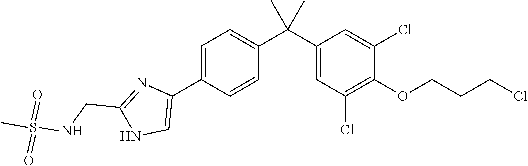 [SMILES: CC(C)(c1ccc(-c2c[nH]c(CNS(C)(=O)=O)n2)cc1)c1cc(Cl)c(OCCCCl)c(Cl)c1] |
| A26 | 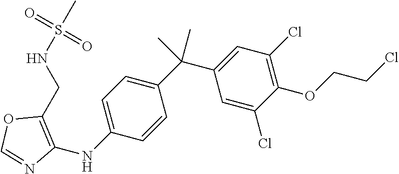 [SMILES: CC(C)(c1ccc(Nc2ncoc2CNS(C)(=O)=O)cc1)c1cc(Cl)c(OCCCl)c(Cl)c1] |
| A27 | 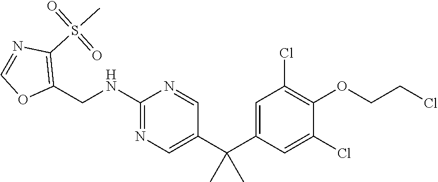 [SMILES: CC(C)(c1cnc(NCc2ocnc2S(C)(=O)=O)nc1)c1cc(Cl)c(OCCCl)c(Cl)c1] |
| A28 | 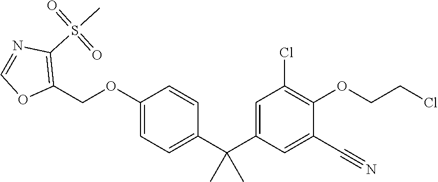 [SMILES: CC(C)(c1ccc(OCc2ocnc2S(C)(=O)=O)cc1)c1cc(Cl)c(OCCCl)c(C#N)c1] |
| A29 | 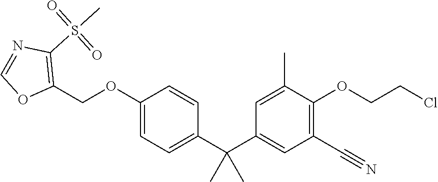 [SMILES: Cc1cc(C(C)(C)c2ccc(OCc3ocnc3S(C)(=O)=O)cc2)cc(C#N)c1OCCCl] |
| A30 | 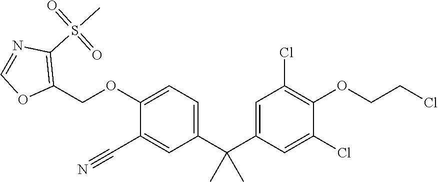 [SMILES: CC(C)(c1cc(Cl)c(OCCCl)c(Cl)c1)c1ccc(OCc2ocnc2S(C)(=O)=O)c(C#N)c1] |
| A31 | 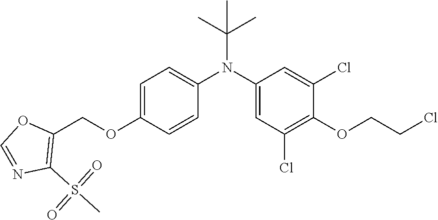 [SMILES: CC(C)(C)N(c1ccc(OCc2ocnc2S(C)(=O)=O)cc1)c1cc(Cl)c(OCCCl)c(Cl)c1] |
| A32 | 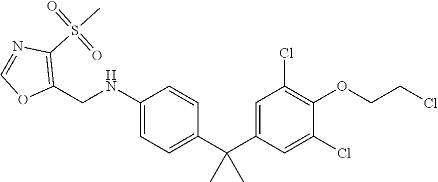 [SMILES: CC(C)(c1ccc(NCc2ocnc2S(C)(=O)=O)cc1)c1cc(Cl)c(OCCCl)c(Cl)c1] |
| A33 | 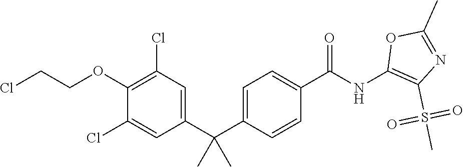 [SMILES: Cc1nc(S(C)(=O)=O)c(NC(=O)c2ccc(C(C)(C)c3cc(Cl)c(OCCCl)c(Cl)c3)cc2)o1] |
| A34 | 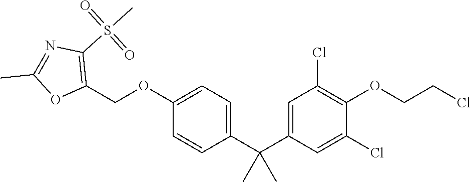 [SMILES: Cc1nc(S(C)(=O)=O)c(COc2ccc(C(C)(C)c3cc(Cl)c(OCCCl)c(Cl)c3)cc2)o1] |
| A35 | 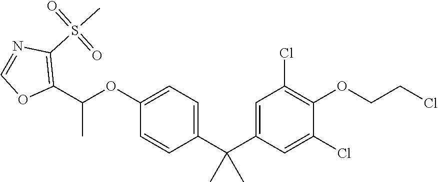 [SMILES: CC(Oc1ccc(C(C)(C)c2cc(Cl)c(OCCCl)c(Cl)c2)cc1)c1ocnc1S(C)(=O)=O] |
| A36 | 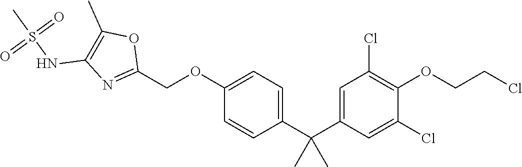 [SMILES: Cc1oc(COc2ccc(C(C)(C)c3cc(Cl)c(OCCCl)c(Cl)c3)cc2)nc1NS(C)(=O)=O] |
| A37 | 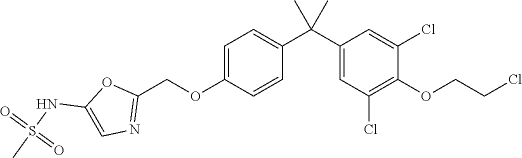 [SMILES: CC(C)(c1ccc(OCc2ncc(NS(C)(=O)=O)o2)cc1)c1cc(Cl)c(OCCCl)c(Cl)c1] |
| A38 | 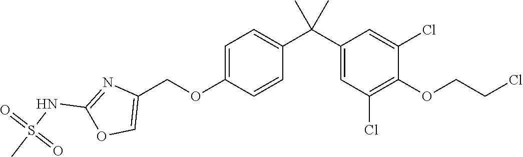 [SMILES: CC(C)(c1ccc(OCc2coc(NS(C)(=O)=O)n2)cc1)c1cc(Cl)c(OCCCl)c(Cl)c1] |
| A39 | 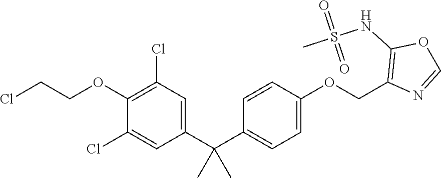 [SMILES: CC(C)(c1ccc(OCc2ncoc2NS(C)(=O)=O)cc1)c1cc(Cl)c(OCCCl)c(Cl)c1] |
| A40 | 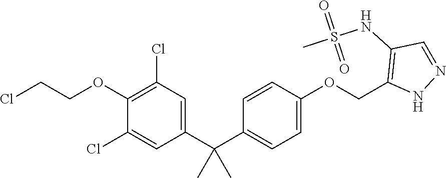 [SMILES: CC(C)(c1ccc(OCc2[nH]ncc2NS(C)(=O)=O)cc1)c1cc(Cl)c(OCCCl)c(Cl)c1] |
| A41 | 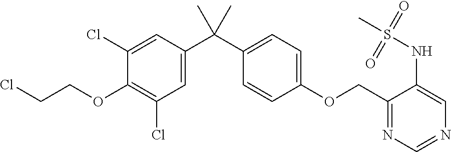 [SMILES: CC(C)(c1ccc(OCc2ncncc2NS(C)(=O)=O)cc1)c1cc(Cl)c(OCCCl)c(Cl)c1] |
| A42 | 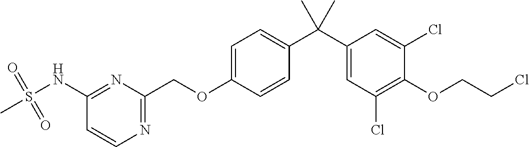 [SMILES: CC(C)(c1ccc(OCc2nccc(NS(C)(=O)=O)n2)cc1)c1cc(Cl)c(OCCCl)c(Cl)c1] |
| A43 | 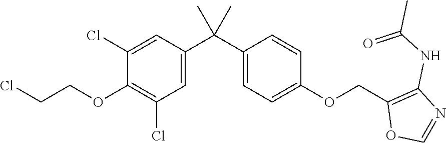 [SMILES: CC(=O)Nc1ncoc1COc1ccc(C(C)(C)c2cc(Cl)c(OCCCl)c(Cl)c2)cc1] |
| A44 | 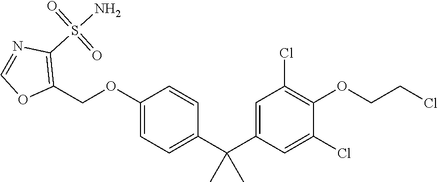 [SMILES: CC(C)(c1ccc(OCc2ocnc2S(N)(=O)=O)cc1)c1cc(Cl)c(OCCCl)c(Cl)c1] |
| A45 | 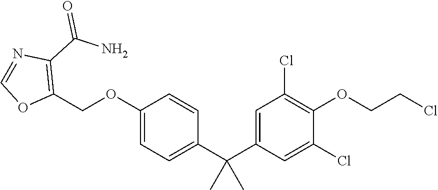 [SMILES: CC(C)(c1ccc(OCc2ocnc2C(N)=O)cc1)c1cc(Cl)c(OCCCl)c(Cl)c1] |
| A46 | 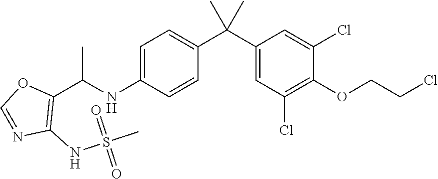 [SMILES: CC(Nc1ccc(C(C)(C)c2cc(Cl)c(OCCCl)c(Cl)c2)cc1)c1ocnc1NS(C)(=O)=O] |
| A47 | 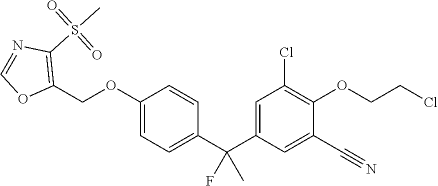 [SMILES: CC(F)(c1ccc(OCc2ocnc2S(C)(=O)=O)cc1)c1cc(Cl)c(OCCCl)c(C#N)c1] |
| A48 | 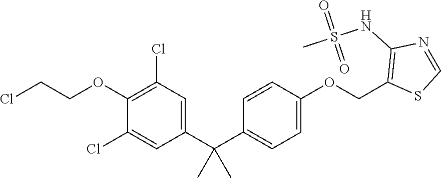 [SMILES: CC(C)(c1ccc(OCc2scnc2NS(C)(=O)=O)cc1)c1cc(Cl)c(OCCCl)c(Cl)c1] |
| A49 | 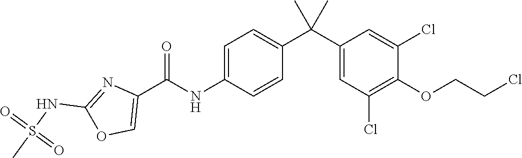 [SMILES: CC(C)(c1ccc(NC(=O)c2coc(NS(C)(=O)=O)n2)cc1)c1cc(Cl)c(OCCCl)c(Cl)c1] |
| A50 | 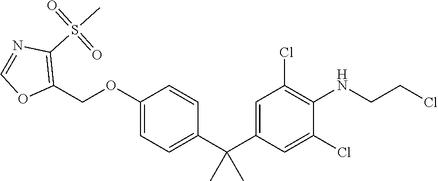 [SMILES: CC(C)(c1ccc(OCc2ocnc2S(C)(=O)=O)cc1)c1cc(Cl)c(NCCCl)c(Cl)c1] |
| A51 | 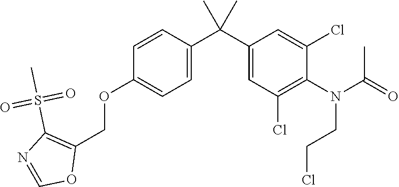 [SMILES: CC(=O)N(CCCl)c1c(Cl)cc(C(C)(C)c2ccc(OCc3ocnc3S(C)(=O)=O)cc2)cc1Cl] |
| A52 | 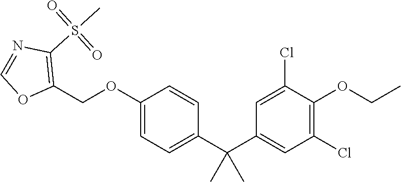 [SMILES: CCOc1c(Cl)cc(C(C)(C)c2ccc(OCc3ocnc3S(C)(=O)=O)cc2)cc1Cl] |
| A53 | 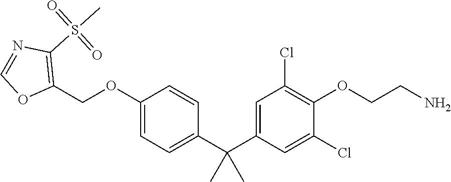 [SMILES: CC(C)(c1ccc(OCc2ocnc2S(C)(=O)=O)cc1)c1cc(Cl)c(OCCN)c(Cl)c1] |
| A54 | 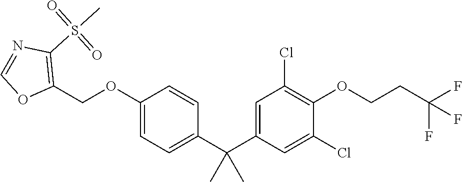 [SMILES: CC(C)(c1ccc(OCc2ocnc2S(C)(=O)=O)cc1)c1cc(Cl)c(OCCC(F)(F)F)c(Cl)c1] |
| A55 | 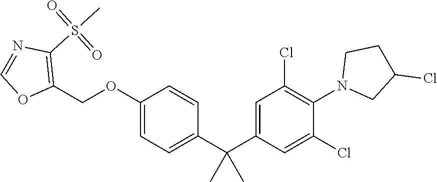 [SMILES: CC(C)(c1ccc(OCc2ocnc2S(C)(=O)=O)cc1)c1cc(Cl)c(N2CCC(Cl)C2)c(Cl)c1] |
| A56 | 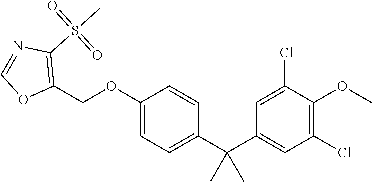 [SMILES: COc1c(Cl)cc(C(C)(C)c2ccc(OCc3ocnc3S(C)(=O)=O)cc2)cc1Cl] |
| A57 | 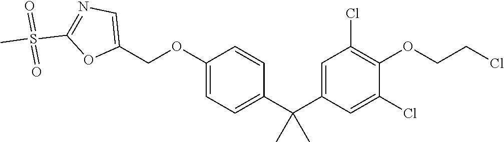 [SMILES: CC(C)(c1ccc(OCc2cnc(S(C)(=O)=O)o2)cc1)c1cc(Cl)c(OCCCl)c(Cl)c1] |
| A58 | 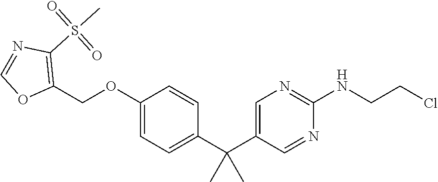 [SMILES: CC(C)(c1ccc(OCc2ocnc2S(C)(=O)=O)cc1)c1cnc(NCCCl)nc1] |
| A59 | 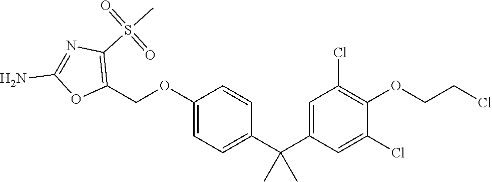 [SMILES: CC(C)(c1ccc(OCc2oc(N)nc2S(C)(=O)=O)cc1)c1cc(Cl)c(OCCCl)c(Cl)c1] |
| A60 | 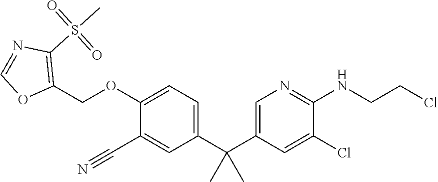 [SMILES: CC(C)(c1cnc(NCCCl)c(Cl)c1)c1ccc(OCc2ocnc2S(C)(=O)=O)c(C#N)c1] |
| A61 | 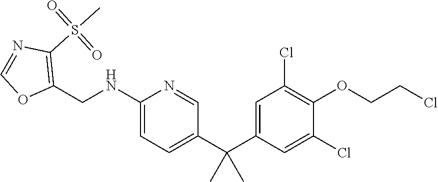 [SMILES: CC(C)(c1ccc(NCc2ocnc2S(C)(=O)=O)nc1)c1cc(Cl)c(OCCCl)c(Cl)c1] |
| A62 | 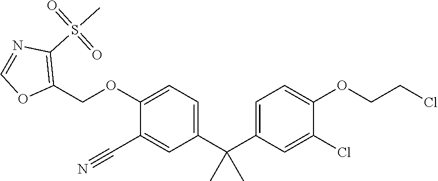 [SMILES: CC(C)(c1ccc(OCCCl)c(Cl)c1)c1ccc(OCc2ocnc2S(C)(=O)=O)c(C#N)c1] |
| A63 | 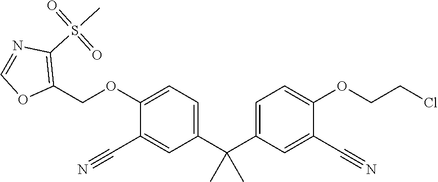 [SMILES: CC(C)(c1ccc(OCCCl)c(C#N)c1)c1ccc(OCc2ocnc2S(C)(=O)=O)c(C#N)c1] |
| A64 | 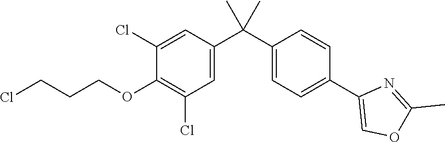 [SMILES: Cc1nc(-c2ccc(C(C)(C)c3cc(Cl)c(OCCCCl)c(Cl)c3)cc2)co1] |
| A65 | 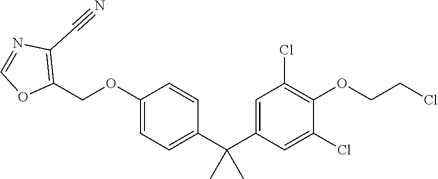 [SMILES: CC(C)(c1ccc(OCc2ocnc2C#N)cc1)c1cc(Cl)c(OCCCl)c(Cl)c1] |
| A66 | 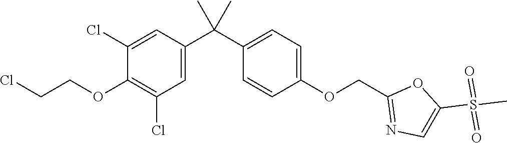 [SMILES: CC(C)(c1ccc(OCc2ncc(S(C)(=O)=O)o2)cc1)c1cc(Cl)c(OCCCl)c(Cl)c1] |
| A67 | 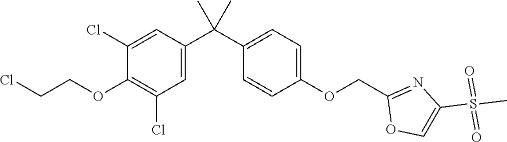 [SMILES: CC(C)(c1ccc(OCc2nc(S(C)(=O)=O)co2)cc1)c1cc(Cl)c(OCCCl)c(Cl)c1] |
| A68 | 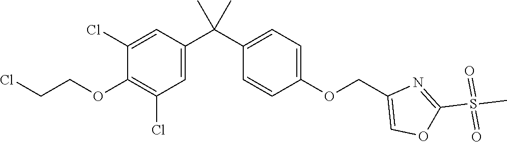 [SMILES: CC(C)(c1ccc(OCc2coc(S(C)(=O)=O)n2)cc1)c1cc(Cl)c(OCCCl)c(Cl)c1] |
| A69 | 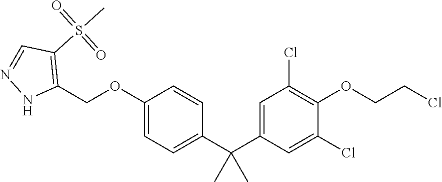 [SMILES: CC(C)(c1ccc(OCc2[nH]ncc2S(C)(=O)=O)cc1)c1cc(Cl)c(OCCCl)c(Cl)c1] |
| A70 | 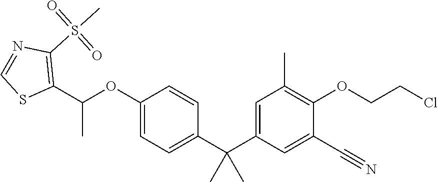 [SMILES: Cc1cc(C(C)(C)c2ccc(OC(C)c3scnc3S(C)(=O)=O)cc2)cc(C#N)c1OCCCl] |
| A71 | 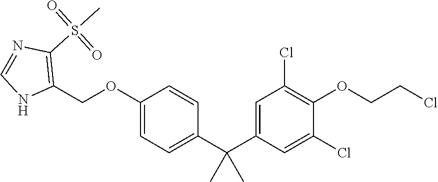 [SMILES: CC(C)(c1ccc(OCc2[nH]cnc2S(C)(=O)=O)cc1)c1cc(Cl)c(OCCCl)c(Cl)c1] |
| A72 | 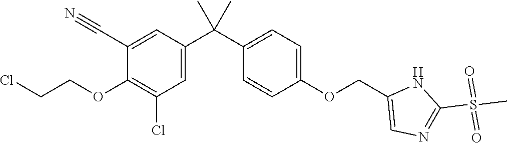 [SMILES: CC(C)(c1ccc(OCc2cnc(S(C)(=O)=O)[nH]2)cc1)c1cc(Cl)c(OCCCl)c(C#N)c1] |
| A73 | 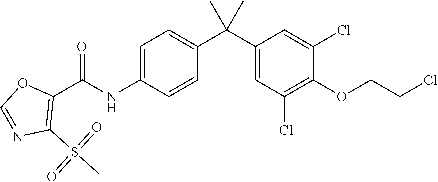 [SMILES: CC(C)(c1ccc(NC(=O)c2ocnc2S(C)(=O)=O)cc1)c1cc(Cl)c(OCCCl)c(Cl)c1] |
| A74 | 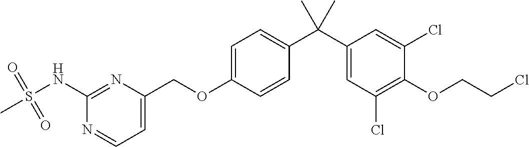 [SMILES: CC(C)(c1ccc(OCc2ccnc(NS(C)(=O)=O)n2)cc1)c1cc(Cl)c(OCCCl)c(Cl)c1] |
| A75 | 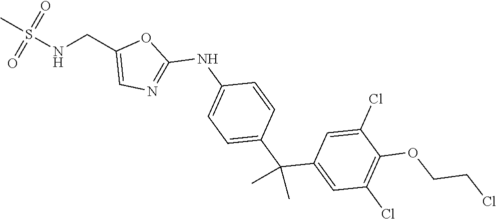 [SMILES: CC(C)(c1ccc(Nc2ncc(CNS(C)(=O)=O)o2)cc1)c1cc(Cl)c(OCCCl)c(Cl)c1] |
| A76 | 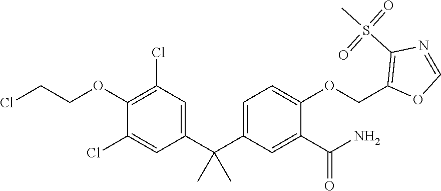 [SMILES: CC(C)(c1cc(Cl)c(OCCCl)c(Cl)c1)c1ccc(OCc2ocnc2S(C)(=O)=O)c(C(N)=O)c1] |
| A77 | 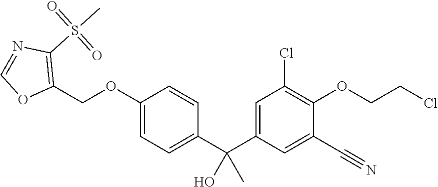 [SMILES: CC(O)(c1ccc(OCc2ocnc2S(C)(=O)=O)cc1)c1cc(Cl)c(OCCCl)c(C#N)c1] |
| A78 | 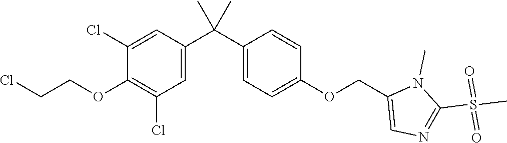 [SMILES: Cn1c(COc2ccc(C(C)(C)c3cc(Cl)c(OCCCl)c(Cl)c3)cc2)cnc1S(C)(=O)=O] |
| A79 | 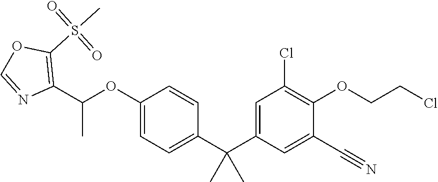 [SMILES: CC(Oc1ccc(C(C)(C)c2cc(Cl)c(OCCCl)c(C#N)c2)cc1)c1ncoc1S(C)(=O)=O] |
| A80 | 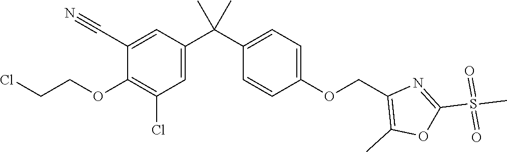 [SMILES: Cc1oc(S(C)(=O)=O)nc1COc1ccc(C(C)(C)c2cc(Cl)c(OCCCl)c(C#N)c2)cc1] |
| A81 | 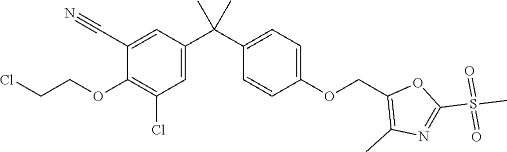 [SMILES: Cc1nc(S(C)(=O)=O)oc1COc1ccc(C(C)(C)c2cc(Cl)c(OCCCl)c(C#N)c2)cc1] |
| A82 | 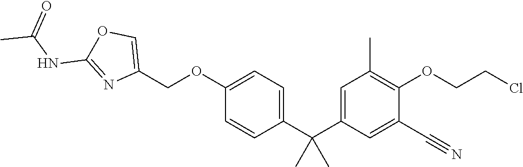 [SMILES: CC(=O)Nc1nc(COc2ccc(C(C)(C)c3cc(C)c(OCCCl)c(C#N)c3)cc2)co1] |
| A83 | 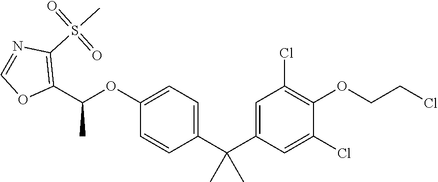 [SMILES: C[C@H](Oc1ccc(C(C)(C)c2cc(Cl)c(OCCCl)c(Cl)c2)cc1)c1ocnc1S(C)(=O)=O] |
| A84 | 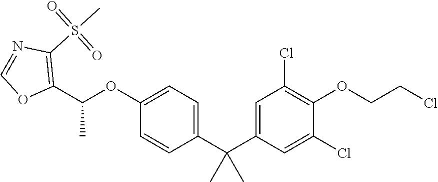 [SMILES: C[C@@H](Oc1ccc(C(C)(C)c2cc(Cl)c(OCCCl)c(Cl)c2)cc1)c1ocnc1S(C)(=O)=O] |
| A85 | 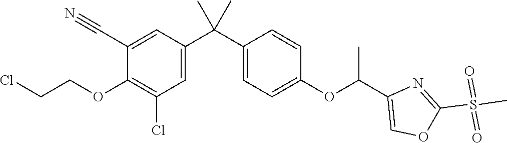 [SMILES: CC(Oc1ccc(C(C)(C)c2cc(Cl)c(OCCCl)c(C#N)c2)cc1)c1coc(S(C)(=O)=O)n1] |
| A86 | 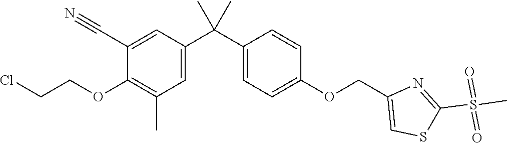 [SMILES: Cc1cc(C(C)(C)c2ccc(OCc3csc(S(C)(=O)=O)n3)cc2)cc(C#N)c1OCCCl] |
| A87 | 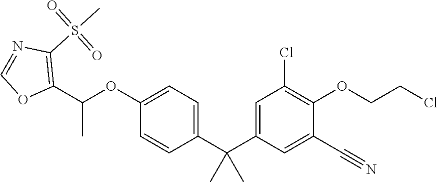 [SMILES: CC(Oc1ccc(C(C)(C)c2cc(Cl)c(OCCCl)c(C#N)c2)cc1)c1ocnc1S(C)(=O)=O] |
| A88 | 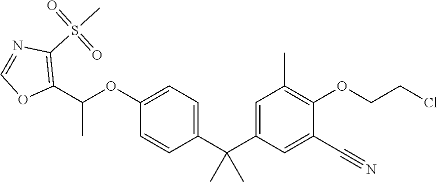 [SMILES: Cc1cc(C(C)(C)c2ccc(OC(C)c3ocnc3S(C)(=O)=O)cc2)cc(C#N)c1OCCCl] |
| A89 | 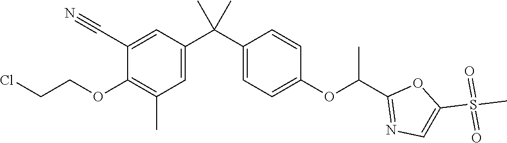 [SMILES: Cc1cc(C(C)(C)c2ccc(OC(C)c3ncc(S(C)(=O)=O)o3)cc2)cc(C#N)c1OCCCl] |
| A90 | 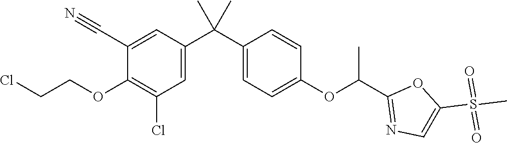 [SMILES: CC(Oc1ccc(C(C)(C)c2cc(Cl)c(OCCCl)c(C#N)c2)cc1)c1ncc(S(C)(=O)=O)o1] |
| A91 | 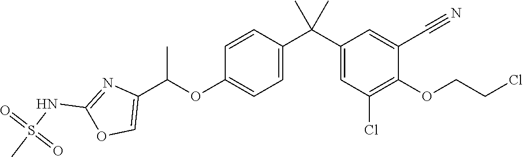 [SMILES: CC(Oc1ccc(C(C)(C)c2cc(Cl)c(OCCCl)c(C#N)c2)cc1)c1coc(NS(C)(=O)=O)n1] |
| A92 | 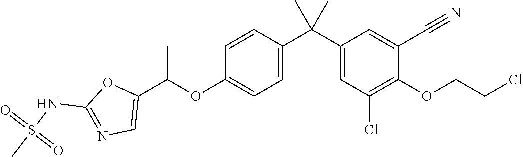 [SMILES: CC(Oc1ccc(C(C)(C)c2cc(Cl)c(OCCCl)c(C#N)c2)cc1)c1cnc(NS(C)(=O)=O)o1] |
| A93 | 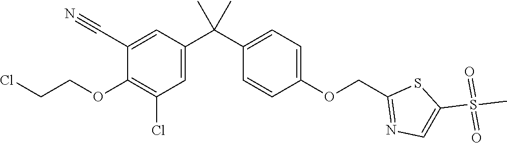 [SMILES: CC(C)(c1ccc(OCc2ncc(S(C)(=O)=O)s2)cc1)c1cc(Cl)c(OCCCl)c(C#N)c1] |
| A94 | 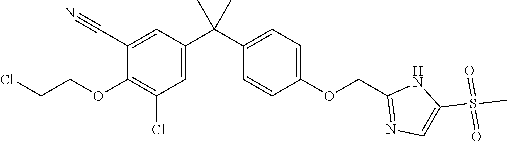 [SMILES: CC(C)(c1ccc(OCc2ncc(S(C)(=O)=O)[nH]2)cc1)c1cc(Cl)c(OCCCl)c(C#N)c1] |
| A95 | 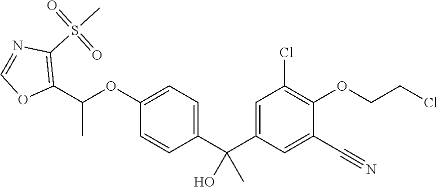 [SMILES: CC(Oc1ccc(C(C)(O)c2cc(Cl)c(OCCCl)c(C#N)c2)cc1)c1ocnc1S(C)(=O)=O] |
| A96 | 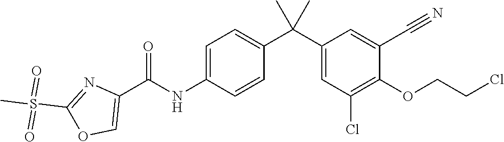 [SMILES: CC(C)(c1ccc(NC(=O)c2coc(S(C)(=O)=O)n2)cc1)c1cc(Cl)c(OCCCl)c(C#N)c1] |
| A97 | 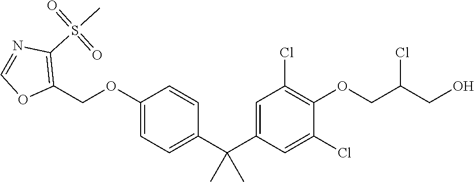 [SMILES: CC(C)(c1ccc(OCc2ocnc2S(C)(=O)=O)cc1)c1cc(Cl)c(OCC(Cl)CO)c(Cl)c1] |
| A98 | 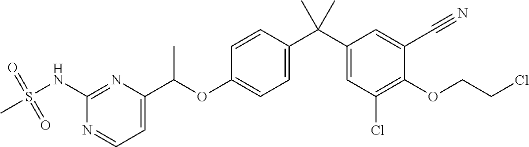 [SMILES: CC(Oc1ccc(C(C)(C)c2cc(Cl)c(OCCCl)c(C#N)c2)cc1)c1ccnc(NS(C)(=O)=O)n1] |
| A99 | 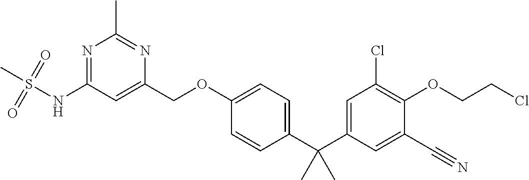 [SMILES: Cc1nc(COc2ccc(C(C)(C)c3cc(Cl)c(OCCCl)c(C#N)c3)cc2)cc(NS(C)(=O)=O)n1] |
| A100 | 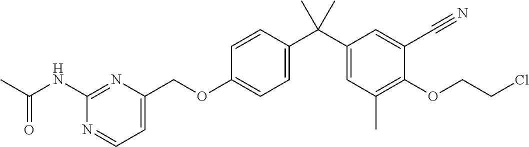 [SMILES: CC(=O)Nc1nccc(COc2ccc(C(C)(C)c3cc(C)c(OCCCl)c(C#N)c3)cc2)n1] |
| A101 | 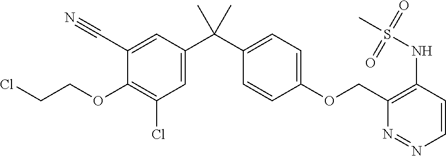 [SMILES: CC(C)(c1ccc(OCc2nnccc2NS(C)(=O)=O)cc1)c1cc(Cl)c(OCCCl)c(C#N)c1] |
| A102 | 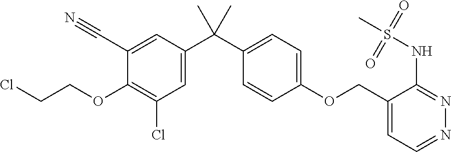 [SMILES: CC(C)(c1ccc(OCc2ccnnc2NS(C)(=O)=O)cc1)c1cc(Cl)c(OCCCl)c(C#N)c1] |
| A103 | 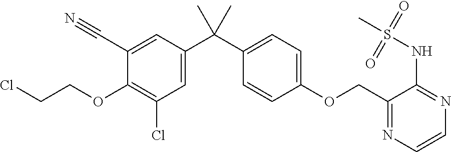 [SMILES: CC(C)(c1ccc(OCc2nccnc2NS(C)(=O)=O)cc1)c1cc(Cl)c(OCCCl)c(C#N)c1] |
| A104 | 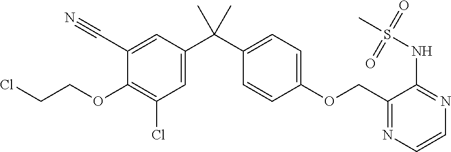 [SMILES: CC(C)(c1ccc(OCc2nccnc2NS(C)(=O)=O)cc1)c1cc(Cl)c(OCCCl)c(C#N)c1] |
| A105 | 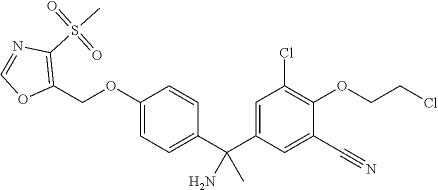 [SMILES: CC(N)(c1ccc(OCc2ocnc2S(C)(=O)=O)cc1)c1cc(Cl)c(OCCCl)c(C#N)c1] |
| A106 | 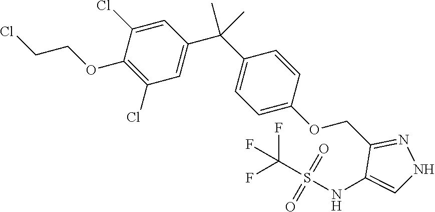 [SMILES: CC(C)(c1ccc(OCc2n[nH]cc2NS(=O)(=O)C(F)(F)F)cc1)c1cc(Cl)c(OCCCl)c(Cl)c1] |
| A107 | 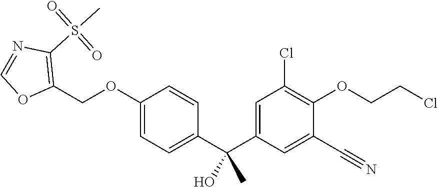 [SMILES: C[C@@](O)(c1ccc(OCc2ocnc2S(C)(=O)=O)cc1)c1cc(Cl)c(OCCCl)c(C#N)c1] |
| A108 | 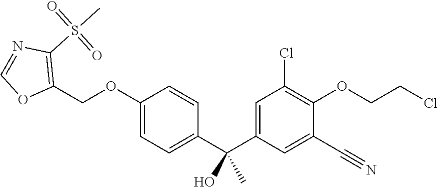 [SMILES: C[C@](O)(c1ccc(OCc2ocnc2S(C)(=O)=O)cc1)c1cc(Cl)c(OCCCl)c(C#N)c1] |
| A109 | 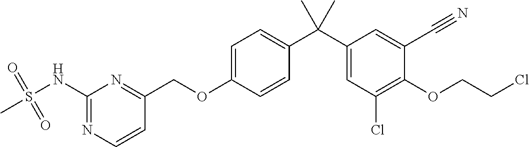 [SMILES: CC(C)(c1ccc(OCc2ccnc(NS(C)(=O)=O)n2)cc1)c1cc(Cl)c(OCCCl)c(C#N)c1] |
| A110 | 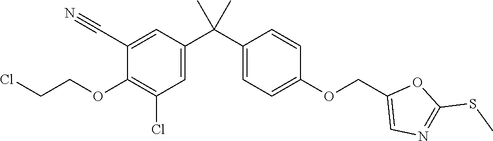 [SMILES: CSc1ncc(COc2ccc(C(C)(C)c3cc(Cl)c(OCCCl)c(C#N)c3)cc2)o1] |
| A111 | 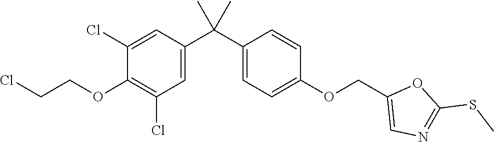 [SMILES: CSc1ncc(COc2ccc(C(C)(C)c3cc(Cl)c(OCCCl)c(Cl)c3)cc2)o1] |
| A112 | 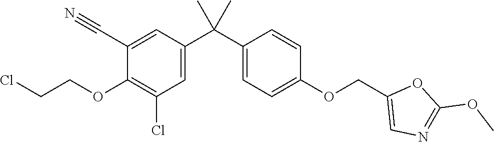 [SMILES: COc1ncc(COc2ccc(C(C)(C)c3cc(Cl)c(OCCCl)c(C#N)c3)cc2)o1] |
| A113 | 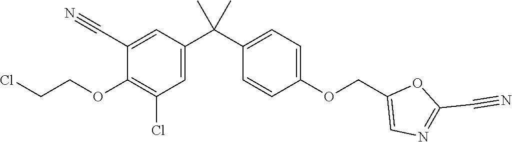 [SMILES: CC(C)(c1ccc(OCc2cnc(C#N)o2)cc1)c1cc(Cl)c(OCCCl)c(C#N)c1] |
| A114 | 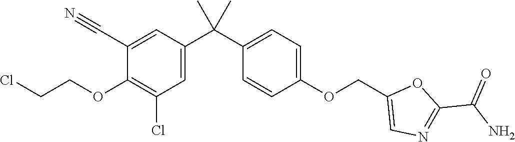 [SMILES: CC(C)(c1ccc(OCc2cnc(C(N)=O)o2)cc1)c1cc(Cl)c(OCCCl)c(C#N)c1] |
| A115 | 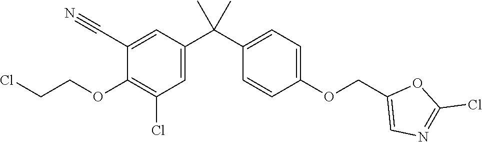 [SMILES: CC(C)(c1ccc(OCc2cnc(Cl)o2)cc1)c1cc(Cl)c(OCCCl)c(C#N)c1] |
| A116 | 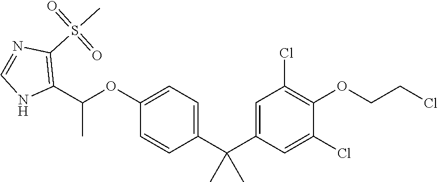 [SMILES: CC(Oc1ccc(C(C)(C)c2cc(Cl)c(OCCCl)c(Cl)c2)cc1)c1[nH]cnc1S(C)(=O)=O] |
| A117 | 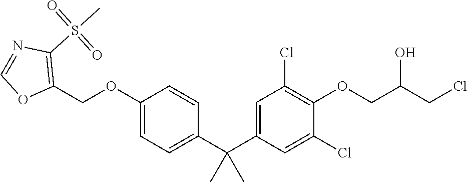 [SMILES: CC(C)(c1ccc(OCc2ocnc2S(C)(=O)=O)cc1)c1cc(Cl)c(OCC(O)CCl)c(Cl)c1] |
| A118 | 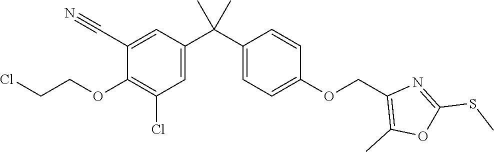 [SMILES: CSc1nc(COc2ccc(C(C)(C)c3cc(Cl)c(OCCCl)c(C#N)c3)cc2)c(C)o1] |
| A119 | 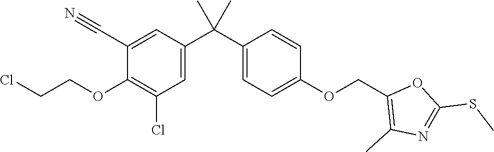 [SMILES: CSc1nc(C)c(COc2ccc(C(C)(C)c3cc(Cl)c(OCCCl)c(C#N)c3)cc2)o1] |
| A120 | 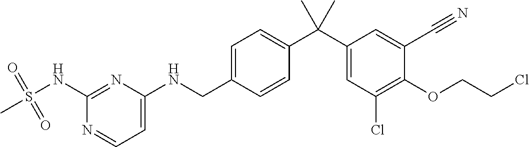 [SMILES: CC(C)(c1ccc(CNc2ccnc(NS(C)(=O)=O)n2)cc1)c1cc(Cl)c(OCCCl)c(C#N)c1] |
| A121 | 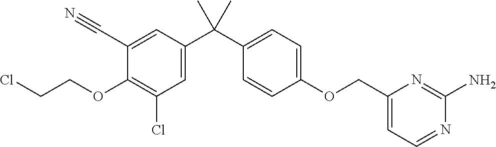 [SMILES: CC(C)(c1ccc(OCc2ccnc(N)n2)cc1)c1cc(Cl)c(OCCCl)c(C#N)c1] |
| A122 | 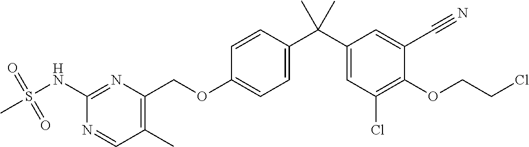 [SMILES: Cc1cnc(NS(C)(=O)=O)nc1COc1ccc(C(C)(C)c2cc(Cl)c(OCCCl)c(C#N)c2)cc1] |
| A123 | 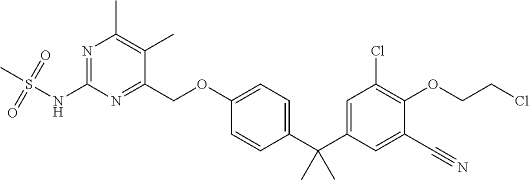 [SMILES: Cc1nc(NS(C)(=O)=O)nc(COc2ccc(C(C)(C)c3cc(Cl)c(OCCCl)c(C#N)c3)cc2)c1C] |
| A124 | 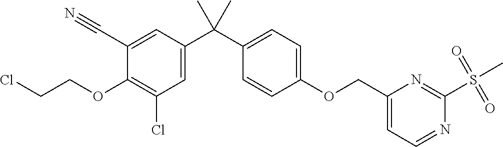 [SMILES: CC(C)(c1ccc(OCc2ccnc(S(C)(=O)=O)n2)cc1)c1cc(Cl)c(OCCCl)c(C#N)c1] |
| A125 | 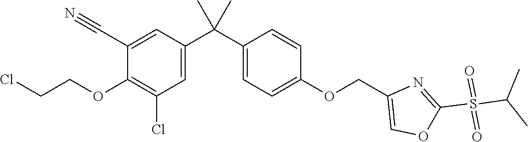 [SMILES: CC(C)S(=O)(=O)c1nc(COc2ccc(C(C)(C)c3cc(Cl)c(OCCCl)c(C#N)c3)cc2)co1] |
| A126 | 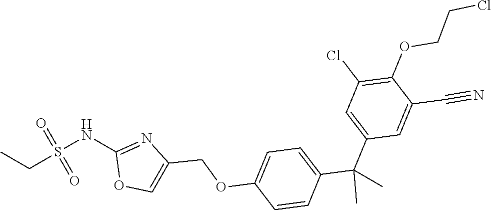 [SMILES: CCS(=O)(=O)Nc1nc(COc2ccc(C(C)(C)c3cc(Cl)c(OCCCl)c(C#N)c3)cc2)co1] |
| A127 | 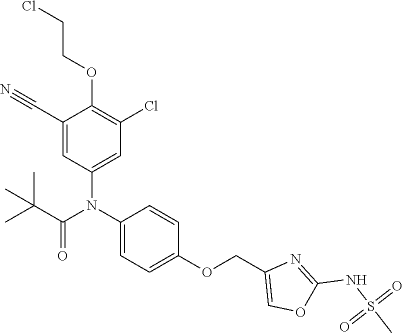 [SMILES: CC(C)(C)C(=O)N(c1ccc(OCc2coc(NS(C)(=O)=O)n2)cc1)c1cc(Cl)c(OCCCl)c(C#N)c1] |
| A128 | 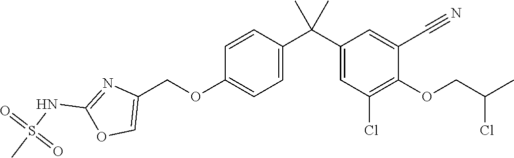 [SMILES: CC(Cl)COc1c(Cl)cc(C(C)(C)c2ccc(OCc3coc(NS(C)(=O)=O)n3)cc2)cc1C#N] |
| A129 | 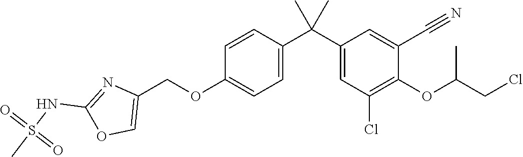 [SMILES: CC(CCl)Oc1c(Cl)cc(C(C)(C)c2ccc(OCc3coc(NS(C)(=O)=O)n3)cc2)cc1C#N] |
| A130 | 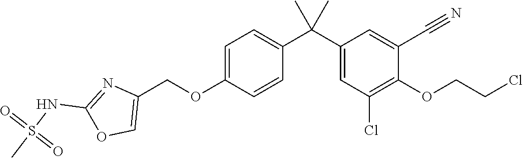 [SMILES: CC(C)(c1ccc(OCc2coc(NS(C)(=O)=O)n2)cc1)c1cc(Cl)c(OCCCl)c(C#N)c1] |
| A131 | 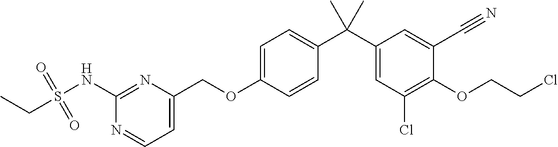 [SMILES: CCS(=O)(=O)Nc1nccc(COc2ccc(C(C)(C)c3cc(Cl)c(OCCCl)c(C#N)c3)cc2)n1] |
| A132 | 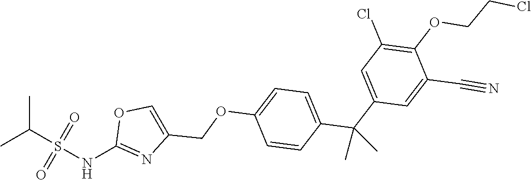 [SMILES: CC(C)S(=O)(=O)Nc1nc(COc2ccc(C(C)(C)c3cc(Cl)c(OCCCl)c(C#N)c3)cc2)co1] |
| A133 | 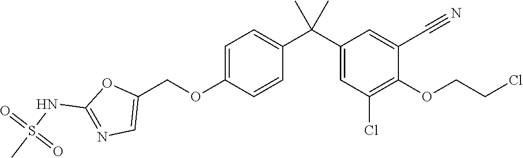 [SMILES: CC(C)(c1ccc(OCc2cnc(NS(C)(=O)=O)o2)cc1)c1cc(Cl)c(OCCCl)c(C#N)c1] |
| A134 | 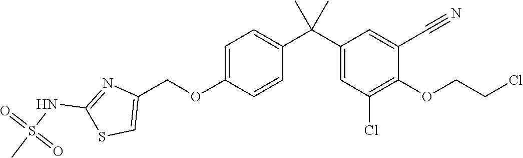 [SMILES: CC(C)(c1ccc(OCc2csc(NS(C)(=O)=O)n2)cc1)c1cc(Cl)c(OCCCl)c(C#N)c1] |
| A135 | 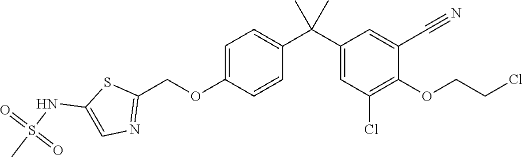 [SMILES: CC(C)(c1ccc(OCc2ncc(NS(C)(=O)=O)s2)cc1)c1cc(Cl)c(OCCCl)c(C#N)c1] |
| A136 | 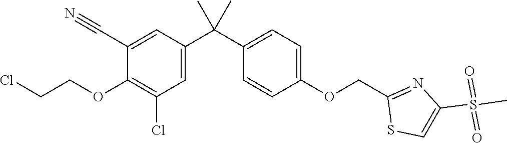 [SMILES: CC(C)(c1ccc(OCc2nc(S(C)(=O)=O)cs2)cc1)c1cc(Cl)c(OCCCl)c(C#N)c1] |
| A137 | 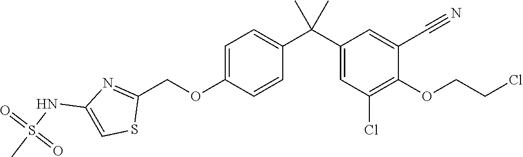 [SMILES: CC(C)(c1ccc(OCc2nc(NS(C)(=O)=O)cs2)cc1)c1cc(Cl)c(OCCCl)c(C#N)c1] |
| A138 | 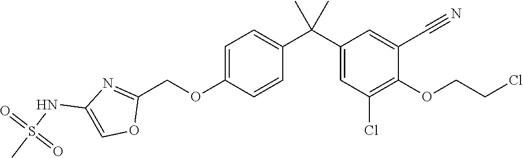 [SMILES: CC(C)(c1ccc(OCc2nc(NS(C)(=O)=O)co2)cc1)c1cc(Cl)c(OCCCl)c(C#N)c1] |
| A139 | 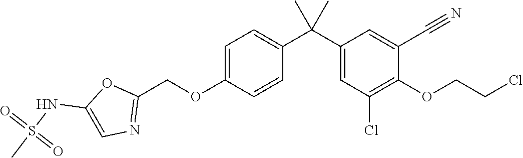 [SMILES: CC(C)(c1ccc(OCc2ncc(NS(C)(=O)=O)o2)cc1)c1cc(Cl)c(OCCCl)c(C#N)c1] |
| A140 | 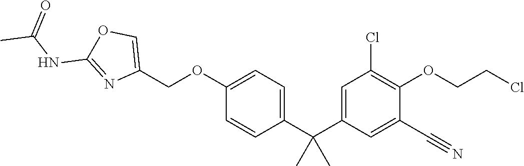 [SMILES: CC(=O)Nc1nc(COc2ccc(C(C)(C)c3cc(Cl)c(OCCCl)c(C#N)c3)cc2)co1] |
| A141 | 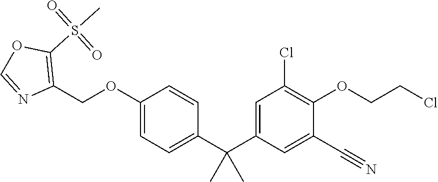 [SMILES: CC(C)(c1ccc(OCc2ncoc2S(C)(=O)=O)cc1)c1cc(Cl)c(OCCCl)c(C#N)c1] |
| A142 | 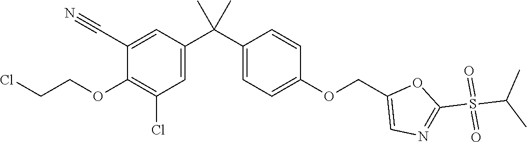 [SMILES: CC(C)S(=O)(=O)c1ncc(COc2ccc(C(C)(C)c3cc(Cl)c(OCCCl)c(C#N)c3)cc2)o1] |
| A143 | 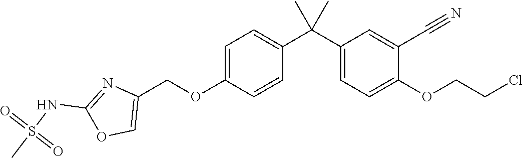 [SMILES: CC(C)(c1ccc(OCc2coc(NS(C)(=O)=O)n2)cc1)c1ccc(OCCCl)c(C#N)c1] |
| A144 | 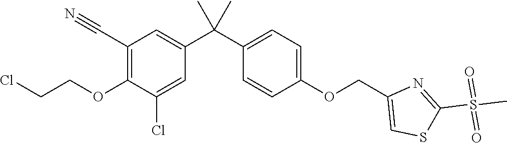 [SMILES: CC(C)(c1ccc(OCc2csc(S(C)(=O)=O)n2)cc1)c1cc(Cl)c(OCCCl)c(C#N)c1] |
| A145 | 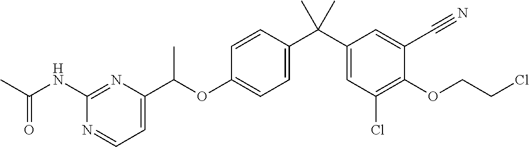 [SMILES: CC(=O)Nc1nccc(C(C)Oc2ccc(C(C)(C)c3cc(Cl)c(OCCCl)c(C#N)c3)cc2)n1] |
| A146 | 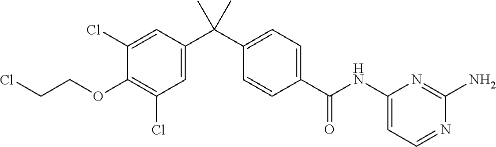 [SMILES: CC(C)(c1ccc(C(=O)Nc2ccnc(N)n2)cc1)c1cc(Cl)c(OCCCl)c(Cl)c1] |
| A147 | 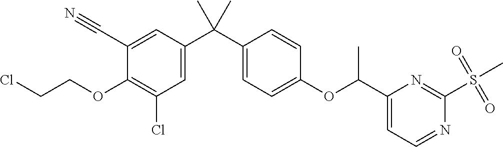 [SMILES: CC(Oc1ccc(C(C)(C)c2cc(Cl)c(OCCCl)c(C#N)c2)cc1)c1ccnc(S(C)(=O)=O)n1] |
| A148 | 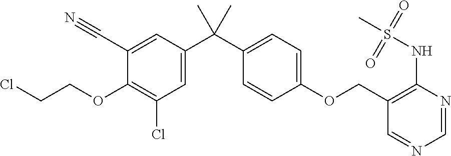 [SMILES: CC(C)(c1ccc(OCc2cncnc2NS(C)(=O)=O)cc1)c1cc(Cl)c(OCCCl)c(C#N)c1] |
| A149 | 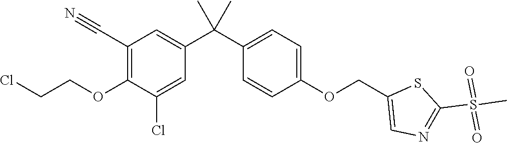 [SMILES: CC(C)(c1ccc(OCc2cnc(S(C)(=O)=O)s2)cc1)c1cc(Cl)c(OCCCl)c(C#N)c1] |
| A150 | 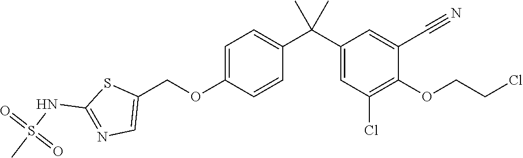 [SMILES: CC(C)(c1ccc(OCc2cnc(NS(C)(=O)=O)s2)cc1)c1cc(Cl)c(OCCCl)c(C#N)c1] |
| A151 | 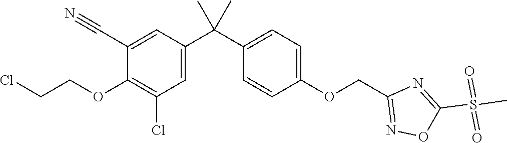 [SMILES: CC(C)(c1ccc(OCc2noc(S(C)(=O)=O)n2)cc1)c1cc(Cl)c(OCCCl)c(C#N)c1] |
| A152 | 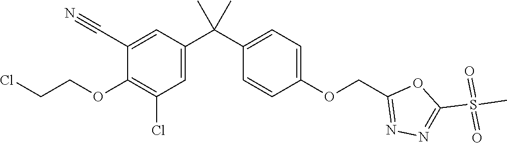 [SMILES: CC(C)(c1ccc(OCc2nnc(S(C)(=O)=O)o2)cc1)c1cc(Cl)c(OCCCl)c(C#N)c1] |
| A153 | 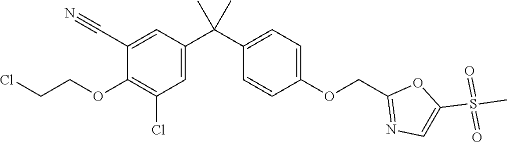 [SMILES: CC(C)(c1ccc(OCc2ncc(S(C)(=O)=O)o2)cc1)c1cc(Cl)c(OCCCl)c(C#N)c1] |
| A154 | 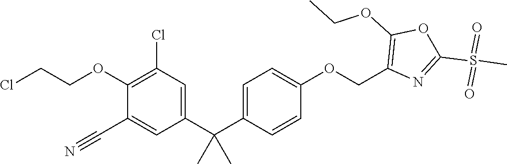 [SMILES: CCOc1oc(S(C)(=O)=O)nc1COc1ccc(C(C)(C)c2cc(Cl)c(OCCCl)c(C#N)c2)cc1] |
| A155 | 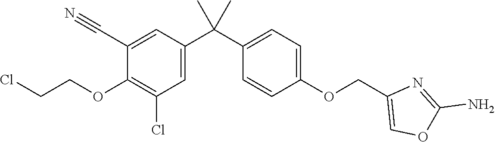 [SMILES: CC(C)(c1ccc(OCc2coc(N)n2)cc1)c1cc(Cl)c(OCCCl)c(C#N)c1] |
| A156 | 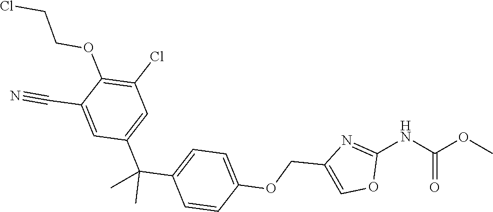 [SMILES: COC(=O)Nc1nc(COc2ccc(C(C)(C)c3cc(Cl)c(OCCCl)c(C#N)c3)cc2)co1] |
| A157 | 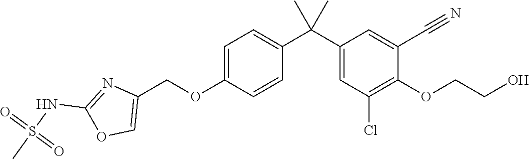 [SMILES: CC(C)(c1ccc(OCc2coc(NS(C)(=O)=O)n2)cc1)c1cc(Cl)c(OCCO)c(C#N)c1] |
| A158 | 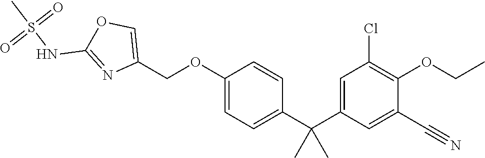 [SMILES: CCOc1c(Cl)cc(C(C)(C)c2ccc(OCc3coc(NS(C)(=O)=O)n3)cc2)cc1C#N] |
| A159 | 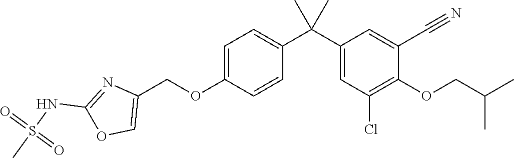 [SMILES: CC(C)COc1c(Cl)cc(C(C)(C)c2ccc(OCc3coc(NS(C)(=O)=O)n3)cc2)cc1C#N] |
| A160 | 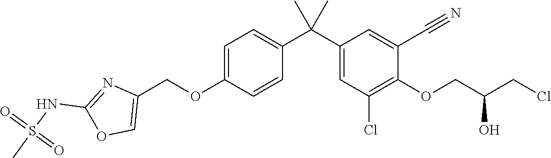 [SMILES: CC(C)(c1ccc(OCc2coc(NS(C)(=O)=O)n2)cc1)c1cc(Cl)c(OC[C@H](O)CCl)c(C#N)c1] |
| A161 | 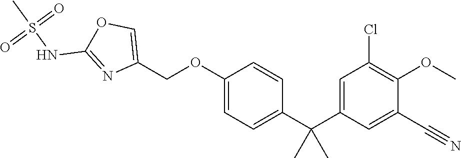 [SMILES: COc1c(Cl)cc(C(C)(C)c2ccc(OCc3coc(NS(C)(=O)=O)n3)cc2)cc1C#N] |
| A162 | 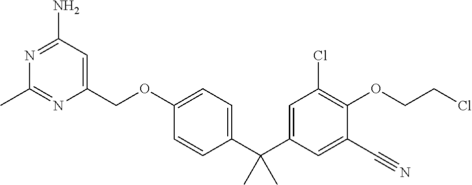 [SMILES: Cc1nc(N)cc(COc2ccc(C(C)(C)c3cc(Cl)c(OCCCl)c(C#N)c3)cc2)n1] |
| A163 | 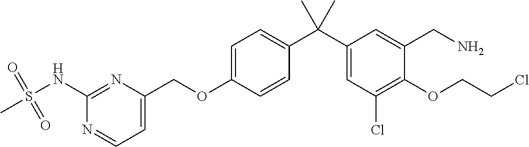 [SMILES: CC(C)(c1ccc(OCc2ccnc(NS(C)(=O)=O)n2)cc1)c1cc(Cl)c(OCCCl)c(CN)c1] |
| A164 | 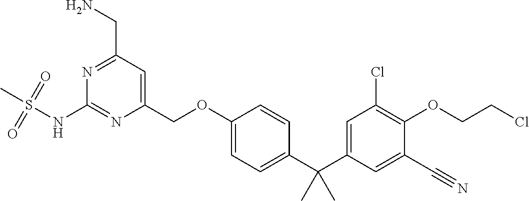 [SMILES: CC(C)(c1ccc(OCc2cc(CN)nc(NS(C)(=O)=O)n2)cc1)c1cc(Cl)c(OCCCl)c(C#N)c1] |
| A165 | 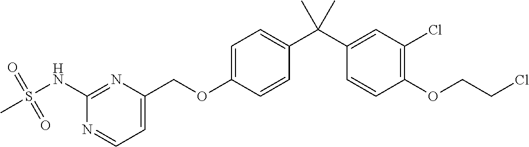 [SMILES: CC(C)(c1ccc(OCc2ccnc(NS(C)(=O)=O)n2)cc1)c1ccc(OCCCl)c(Cl)c1] |
| A166 | 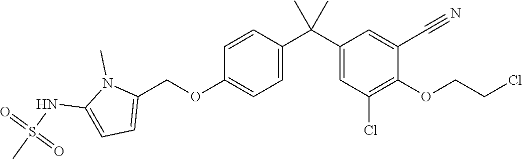 [SMILES: Cn1c(COc2ccc(C(C)(C)c3cc(Cl)c(OCCCl)c(C#N)c3)cc2)ccc1NS(C)(=O)=O] |
| A167 | 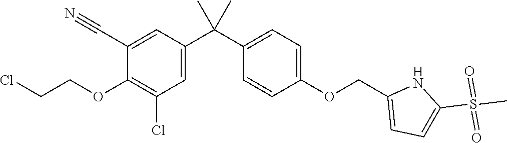 [SMILES: CC(C)(c1ccc(OCc2ccc(S(C)(=O)=O)[nH]2)cc1)c1cc(Cl)c(OCCCl)c(C#N)c1] |
| A168 | 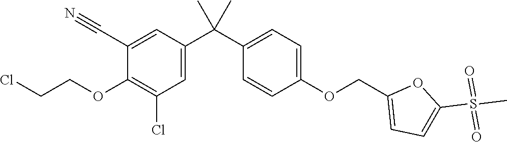 [SMILES: CC(C)(c1ccc(OCc2ccc(S(C)(=O)=O)o2)cc1)c1cc(Cl)c(OCCCl)c(C#N)c1] |
| A169 | 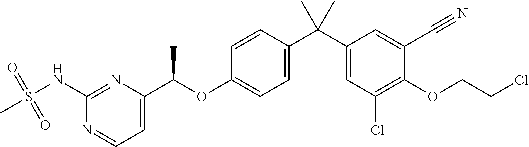 [SMILES: C[C@@H](Oc1ccc(C(C)(C)c2cc(Cl)c(OCCCl)c(C#N)c2)cc1)c1ccnc(NS(C)(=O)=O)n1] |
| A170 | 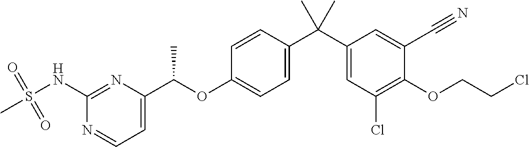 [SMILES: C[C@H](Oc1ccc(C(C)(C)c2cc(Cl)c(OCCCl)c(C#N)c2)cc1)c1ccnc(NS(C)(=O)=O)n1] |
| A171 | 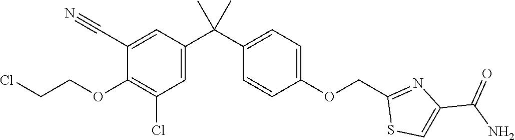 [SMILES: CC(C)(c1ccc(OCc2nc(C(N)=O)cs2)cc1)c1cc(Cl)c(OCCCl)c(C#N)c1] |
| A172 | 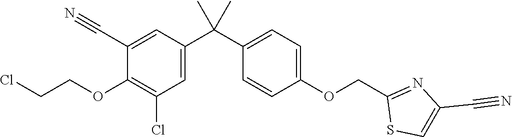 [SMILES: CC(C)(c1ccc(OCc2nc(C#N)cs2)cc1)c1cc(Cl)c(OCCCl)c(C#N)c1] |
| A173 | 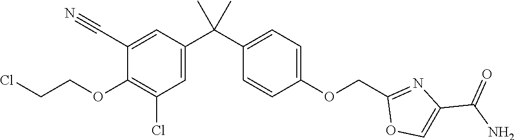 [SMILES: CC(C)(c1ccc(OCc2nc(C(N)=O)co2)cc1)c1cc(Cl)c(OCCCl)c(C#N)c1] |
| A174 | 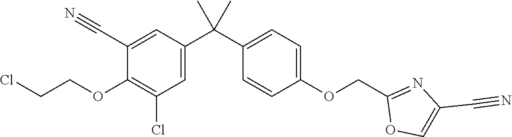 [SMILES: CC(C)(c1ccc(OCc2nc(C#N)co2)cc1)c1cc(Cl)c(OCCCl)c(C#N)c1] |
| A175 | 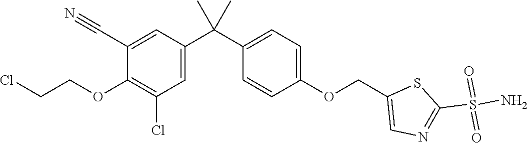 [SMILES: CC(C)(c1ccc(OCc2cnc(S(N)(=O)=O)s2)cc1)c1cc(Cl)c(OCCCl)c(C#N)c1] |
| A176 | 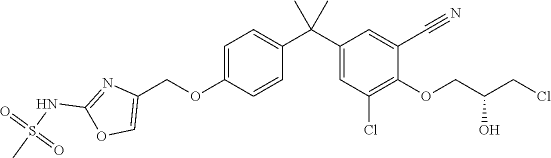 [SMILES: CC(C)(c1ccc(OCc2coc(NS(C)(=O)=O)n2)cc1)c1cc(Cl)c(OC[C@@H](O)CCl)c(C#N)c1] |
| A177 | 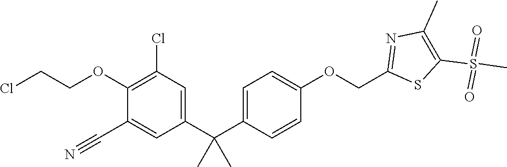 [SMILES: Cc1nc(COc2ccc(C(C)(C)c3cc(Cl)c(OCCCl)c(C#N)c3)cc2)sc1S(C)(=O)=O] |
| A178 | 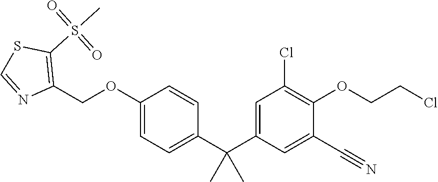 [SMILES: CC(C)(c1ccc(OCc2ncsc2S(C)(=O)=O)cc1)c1cc(Cl)c(OCCCl)c(C#N)c1] |
| A179 | 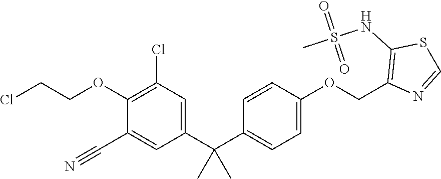 [SMILES: CC(C)(c1ccc(OCc2ncsc2NS(C)(=O)=O)cc1)c1cc(Cl)c(OCCCl)c(C#N)c1] |
| A180 | 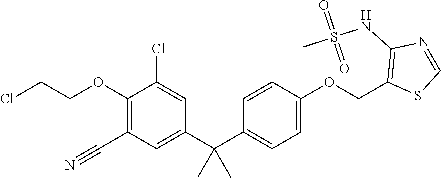 [SMILES: CC(C)(c1ccc(OCc2scnc2NS(C)(=O)=O)cc1)c1cc(Cl)c(OCCCl)c(C#N)c1] |
| A181 | 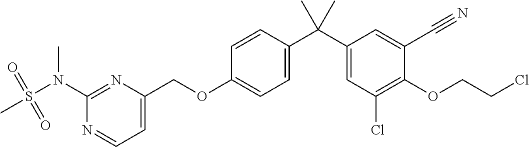 [SMILES: CN(c1nccc(COc2ccc(C(C)(C)c3cc(Cl)c(OCCCl)c(C#N)c3)cc2)n1)S(C)(=O)=O] |
| A182 | 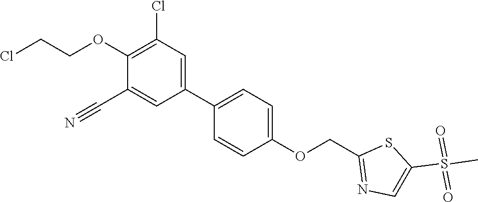 [SMILES: CS(=O)(=O)c1cnc(COc2ccc(-c3cc(Cl)c(OCCCl)c(C#N)c3)cc2)s1] |
| A183 | 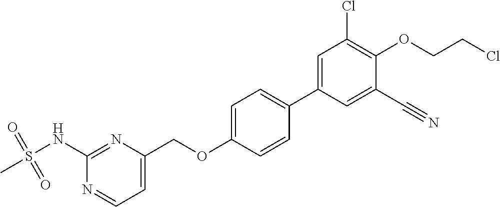 [SMILES: CS(=O)(=O)Nc1nccc(COc2ccc(-c3cc(Cl)c(OCCCl)c(C#N)c3)cc2)n1] |
| A184 | 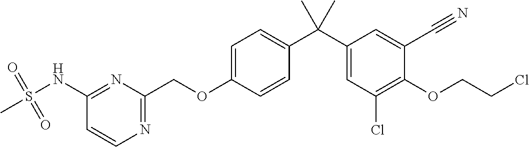 [SMILES: CC(C)(c1ccc(OCc2nccc(NS(C)(=O)=O)n2)cc1)c1cc(Cl)c(OCCCl)c(C#N)c1] |
| A185 | 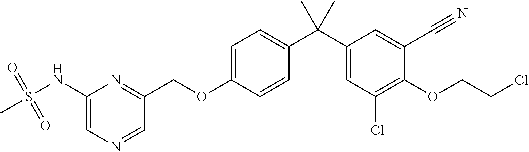 [SMILES: CC(C)(c1ccc(OCc2cncc(NS(C)(=O)=O)n2)cc1)c1cc(Cl)c(OCCCl)c(C#N)c1] |
| A186 | 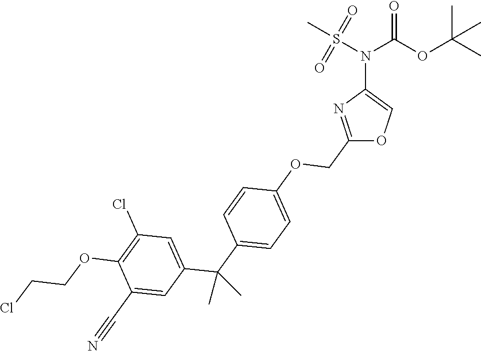 [SMILES: CC(C)(C)OC(=O)N(c1coc(COc2ccc(C(C)(C)c3cc(Cl)c(OCCCl)c(C#N)c3)cc2)n1)S(C)(=O)=O] |
| A187 | 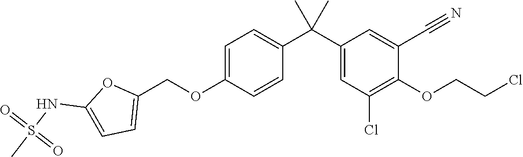 [SMILES: CC(C)(c1ccc(OCc2ccc(NS(C)(=O)=O)o2)cc1)c1cc(Cl)c(OCCCl)c(C#N)c1] |
| A188 | 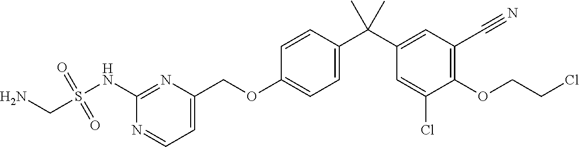 [SMILES: CC(C)(c1ccc(OCc2ccnc(NS(=O)(=O)CN)n2)cc1)c1cc(Cl)c(OCCCl)c(C#N)c1] |
| A189 | 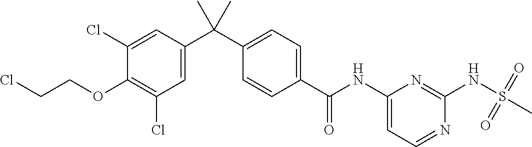 [SMILES: CC(C)(c1ccc(C(=O)Nc2ccnc(NS(C)(=O)=O)n2)cc1)c1cc(Cl)c(OCCCl)c(Cl)c1] |
| A190 | 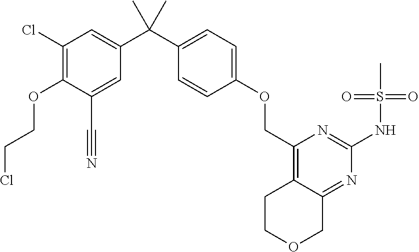 [SMILES: CC(C)(c1ccc(OCc2nc(NS(C)(=O)=O)nc3c2CCOC3)cc1)c1cc(Cl)c(OCCCl)c(C#N)c1] |
| A191 | 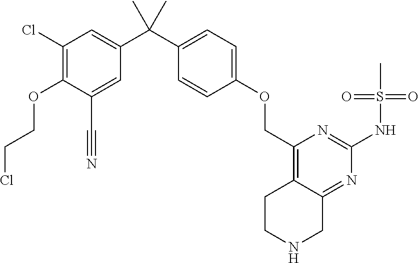 [SMILES: CC(C)(c1ccc(OCc2nc(NS(C)(=O)=O)nc3c2CCNC3)cc1)c1cc(Cl)c(OCCCl)c(C#N)c1] |
| A192 | 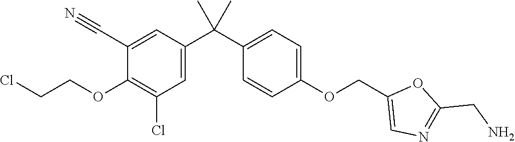 [SMILES: CC(C)(c1ccc(OCc2cnc(CN)o2)cc1)c1cc(Cl)c(OCCCl)c(C#N)c1] |
| A193 | 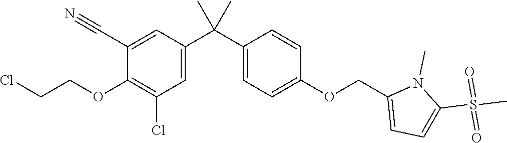 [SMILES: Cn1c(COc2ccc(C(C)(C)c3cc(Cl)c(OCCCl)c(C#N)c3)cc2)ccc1S(C)(=O)=O] |
| A194 | 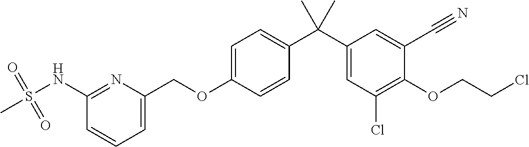 [SMILES: CC(C)(c1ccc(OCc2cccc(NS(C)(=O)=O)n2)cc1)c1cc(Cl)c(OCCCl)c(C#N)c1] |
| A195 | 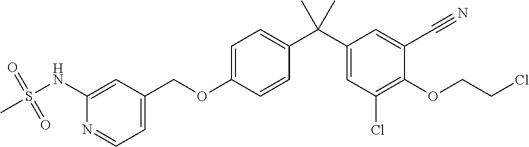 [SMILES: CC(C)(c1ccc(OCc2ccnc(NS(C)(=O)=O)c2)cc1)c1cc(Cl)c(OCCCl)c(C#N)c1] |
| A196 | 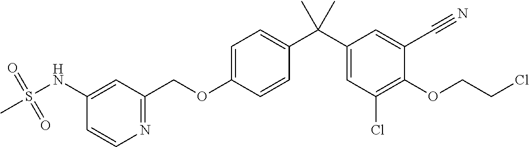 [SMILES: CC(C)(c1ccc(OCc2cc(NS(C)(=O)=O)ccn2)cc1)c1cc(Cl)c(OCCCl)c(C#N)c1] |
| A197 | 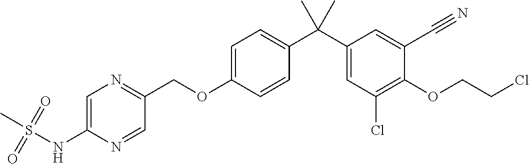 [SMILES: CC(C)(c1ccc(OCc2cnc(NS(C)(=O)=O)cn2)cc1)c1cc(Cl)c(OCCCl)c(C#N)c1] |
| A198 | 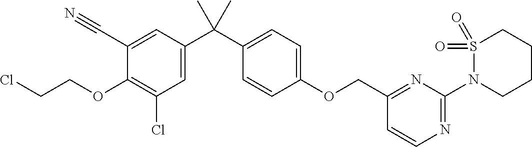 [SMILES: CC(C)(c1ccc(OCc2ccnc(N3CCCCS3(=O)=O)n2)cc1)c1cc(Cl)c(OCCCl)c(C#N)c1] |
| A199 | 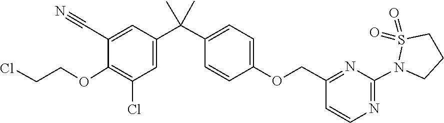 [SMILES: CC(C)(c1ccc(OCc2ccnc(N3CCCS3(=O)=O)n2)cc1)c1cc(Cl)c(OCCCl)c(C#N)c1] |
| A200 | 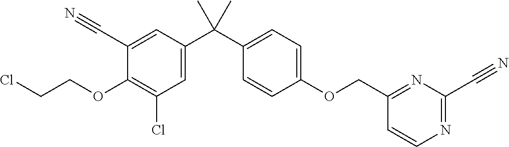 [SMILES: CC(C)(c1ccc(OCc2ccnc(C#N)n2)cc1)c1cc(Cl)c(OCCCl)c(C#N)c1] |
| A201 | 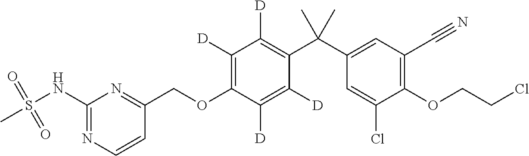 [SMILES: [2H]c1c([2H])c(C(C)(C)c2cc(Cl)c(OCCCl)c(C#N)c2)c([2H])c([2H])c1OCc1ccnc(NS(C)(=O)=O)n1] |
| A202 | 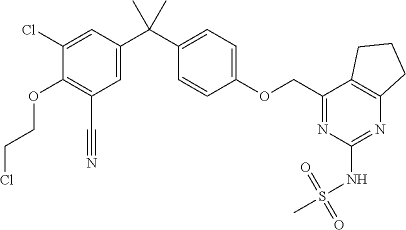 [SMILES: CC(C)(c1ccc(OCc2nc(NS(C)(=O)=O)nc3c2CCC3)cc1)c1cc(Cl)c(OCCCl)c(C#N)c1] |
| A203 | 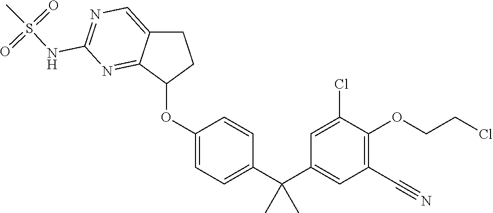 [SMILES: CC(C)(c1ccc(OC2CCc3cnc(NS(C)(=O)=O)nc32)cc1)c1cc(Cl)c(OCCCl)c(C#N)c1] |
| A204 | 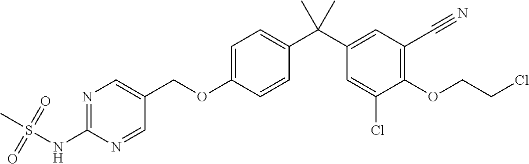 [SMILES: CC(C)(c1ccc(OCc2cnc(NS(C)(=O)=O)nc2)cc1)c1cc(Cl)c(OCCCl)c(C#N)c1] |
| A205 | 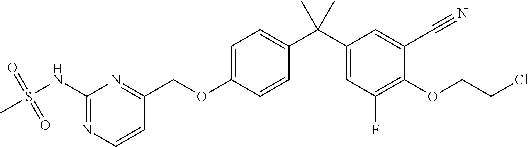 [SMILES: CC(C)(c1ccc(OCc2ccnc(NS(C)(=O)=O)n2)cc1)c1cc(F)c(OCCCl)c(C#N)c1] |
| A206 | 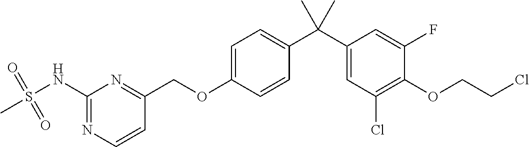 [SMILES: CC(C)(c1ccc(OCc2ccnc(NS(C)(=O)=O)n2)cc1)c1cc(F)c(OCCCl)c(Cl)c1] |
| A207 | 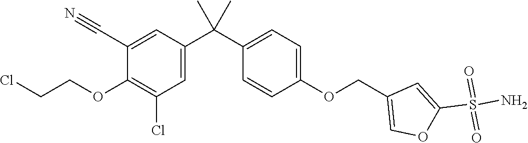 [SMILES: CC(C)(c1ccc(OCc2coc(S(N)(=O)=O)c2)cc1)c1cc(Cl)c(OCCCl)c(C#N)c1] |
| A208 | 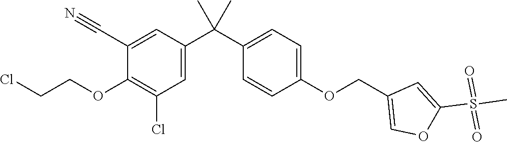 [SMILES: CC(C)(c1ccc(OCc2coc(S(C)(=O)=O)c2)cc1)c1cc(Cl)c(OCCCl)c(C#N)c1] |
| A209 | 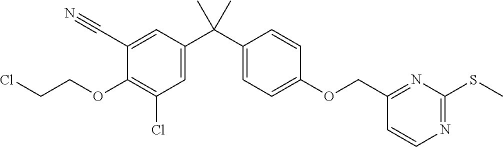 [SMILES: CSc1nccc(COc2ccc(C(C)(C)c3cc(Cl)c(OCCCl)c(C#N)c3)cc2)n1] |
| A210 | 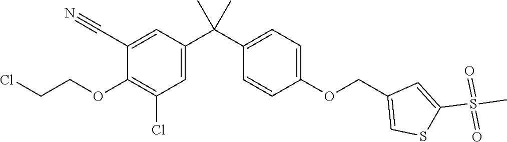 [SMILES: CC(C)(c1ccc(OCc2csc(S(C)(=O)=O)c2)cc1)c1cc(Cl)c(OCCCl)c(C#N)c1] |
| A211 | 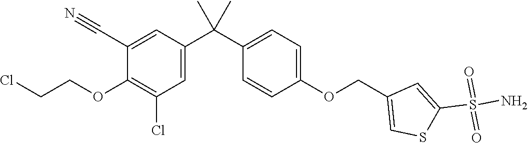 [SMILES: CC(C)(c1ccc(OCc2csc(S(N)(=O)=O)c2)cc1)c1cc(Cl)c(OCCCl)c(C#N)c1] |
| A212 | 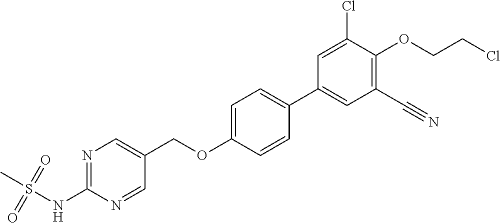 [SMILES: CS(=O)(=O)Nc1ncc(COc2ccc(-c3cc(Cl)c(OCCCl)c(C#N)c3)cc2)cn1] |
| A213 | 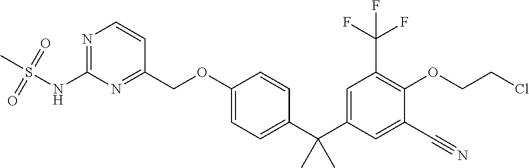 [SMILES: CC(C)(c1ccc(OCc2ccnc(NS(C)(=O)=O)n2)cc1)c1cc(C#N)c(OCCCl)c(C(F)(F)F)c1] |
| A214 | 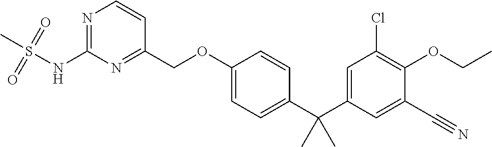 [SMILES: CCOc1c(Cl)cc(C(C)(C)c2ccc(OCc3ccnc(NS(C)(=O)=O)n3)cc2)cc1C#N] |
| A215 | 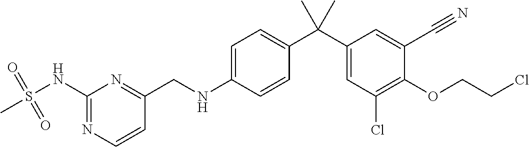 [SMILES: CC(C)(c1ccc(NCc2ccnc(NS(C)(=O)=O)n2)cc1)c1cc(Cl)c(OCCCl)c(C#N)c1] |
| A216 | 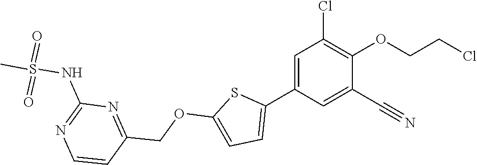 [SMILES: CS(=O)(=O)Nc1nccc(COc2ccc(-c3cc(Cl)c(OCCCl)c(C#N)c3)s2)n1] |
| A217 | 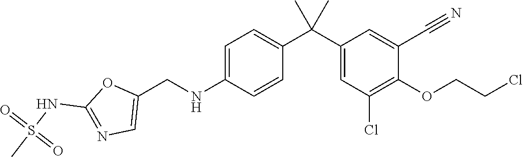 [SMILES: CC(C)(c1ccc(NCc2cnc(NS(C)(=O)=O)o2)cc1)c1cc(Cl)c(OCCCl)c(C#N)c1] |
| A218 | 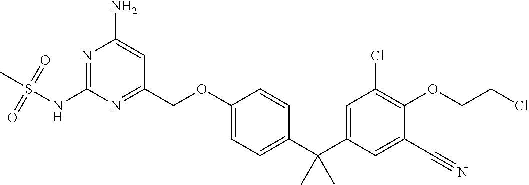 [SMILES: CC(C)(c1ccc(OCc2cc(N)nc(NS(C)(=O)=O)n2)cc1)c1cc(Cl)c(OCCCl)c(C#N)c1] |
| A219 | 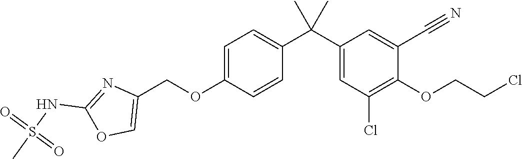 [SMILES: CC(C)(c1ccc(OCc2coc(NS(C)(=O)=O)n2)cc1)c1cc(Cl)c(OCCCl)c(C#N)c1] |
| A220 | 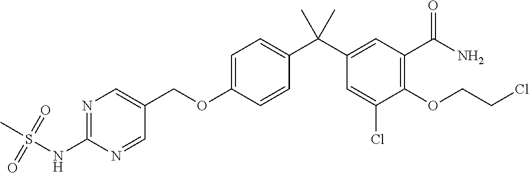 [SMILES: CC(C)(c1ccc(OCc2cnc(NS(C)(=O)=O)nc2)cc1)c1cc(Cl)c(OCCCl)c(C(N)=O)c1] |
| A221 | 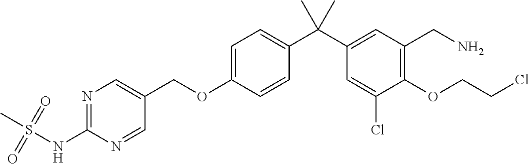 [SMILES: CC(C)(c1ccc(OCc2cnc(NS(C)(=O)=O)nc2)cc1)c1cc(Cl)c(OCCCl)c(CN)c1] |
| A222 | 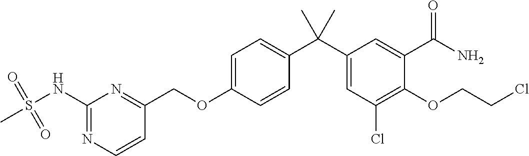 [SMILES: CC(C)(c1ccc(OCc2ccnc(NS(C)(=O)=O)n2)cc1)c1cc(Cl)c(OCCCl)c(C(N)=O)c1] |
| A223 | 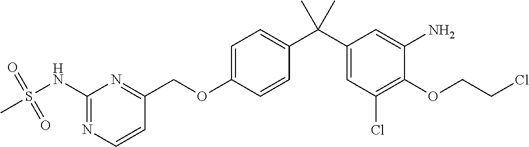 [SMILES: CC(C)(c1ccc(OCc2ccnc(NS(C)(=O)=O)n2)cc1)c1cc(N)c(OCCCl)c(Cl)c1] |
| A224 | 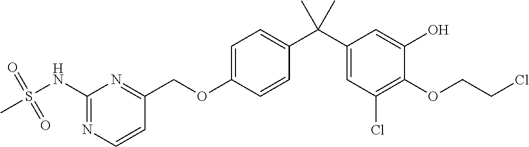 [SMILES: CC(C)(c1ccc(OCc2ccnc(NS(C)(=O)=O)n2)cc1)c1cc(O)c(OCCCl)c(Cl)c1] |
| A225 | 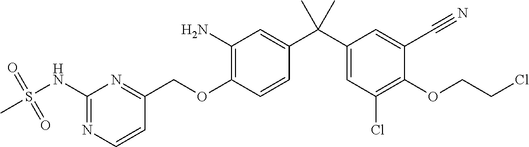 [SMILES: CC(C)(c1ccc(OCc2ccnc(NS(C)(=O)=O)n2)c(N)c1)c1cc(Cl)c(OCCCl)c(C#N)c1] |
| A226 | 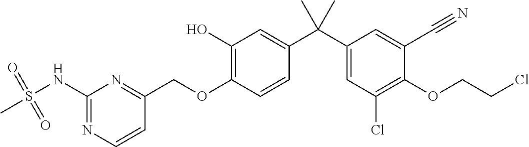 [SMILES: CC(C)(c1ccc(OCc2ccnc(NS(C)(=O)=O)n2)c(O)c1)c1cc(Cl)c(OCCCl)c(C#N)c1] |
| A227 | 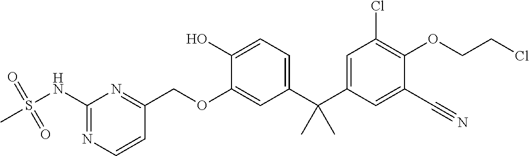 [SMILES: CC(C)(c1cc(Cl)c(OCCCl)c(C#N)c1)c1ccc(O)c(OCc2ccnc(NS(C)(=O)=O)n2)c1] |
| A228 | 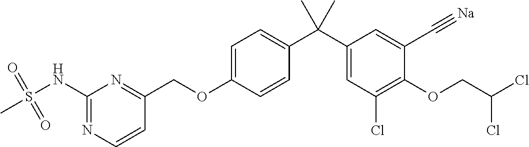 |
| A229 | 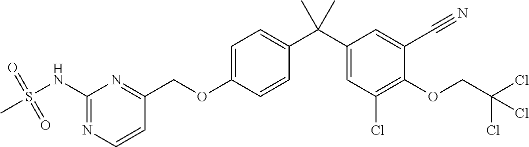 [SMILES: CC(C)(c1ccc(OCc2ccnc(NS(C)(=O)=O)n2)cc1)c1cc(Cl)c(OCC(Cl)(Cl)Cl)c(C#N)c1] |
| A230 | 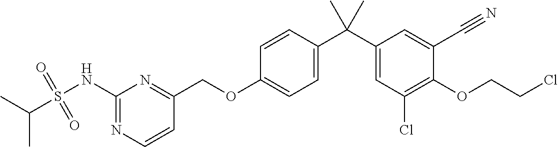 [SMILES: CC(C)S(=O)(=O)Nc1nccc(COc2ccc(C(C)(C)c3cc(Cl)c(OCCCl)c(C#N)c3)cc2)n1] |
| A231 | 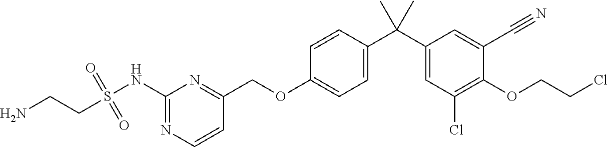 [SMILES: CC(C)(c1ccc(OCc2ccnc(NS(=O)(=O)CCN)n2)cc1)c1cc(Cl)c(OCCCl)c(C#N)c1] |
| A232 | 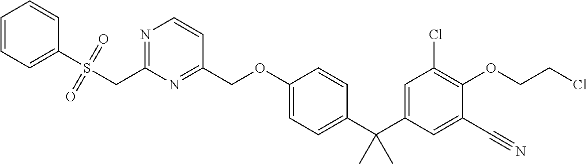 [SMILES: CC(C)(c1ccc(OCc2ccnc(CS(=O)(=O)c3ccccc3)n2)cc1)c1cc(Cl)c(OCCCl)c(C#N)c1] |
| A233 | 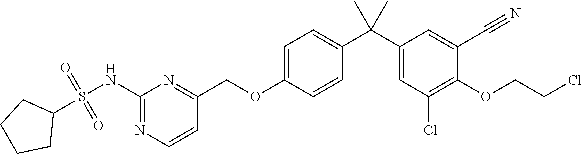 [SMILES: CC(C)(c1ccc(OCc2ccnc(NS(=O)(=O)C3CCCC3)n2)cc1)c1cc(Cl)c(OCCCl)c(C#N)c1] |
| A234 | 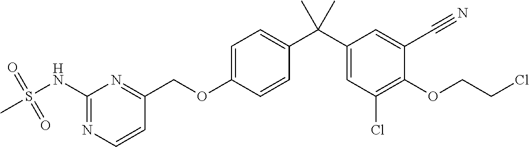 [SMILES: CC(C)(c1ccc(OCc2ccnc(NS(C)(=O)=O)n2)cc1)c1cc(Cl)c(OCCCl)c(C#N)c1] |

In one embodiment of the PTC of formula (I)-(IV), (VI), (B) or (C), the PTC is selected from Table B below, or a pharmaceutically acceptable salt, tautomer, stereoisomer or prodrug thereof. In one embodiment of the PTC of formula (I)-(IV), (VI), (B) or (C), the PTC is selected from PTCs B1, B2, B3, or B6 or a pharmaceutically acceptable salt, tautomer, stereoisomer or prodrug thereof. In one embodiment of the PTC of formula (I)-(IV), (VI), (B) or (C), the PTC is selected from PTCs B4, B5, B7, B8, B9, B10, or B11 or a pharmaceutically acceptable salt, tautomer, stereoisomer or prodrug thereof. In one embodiment of the PTC of formula (I)-(IV), (VI), (B) or (C), the PTC is selected from PTCs B1-B11 or a pharmaceutically acceptable salt, tautomer, stereoisomer or prodrug thereof.

TABLE B
PTCs

| PTC ID | Structure |
|---|---|
| B1 | 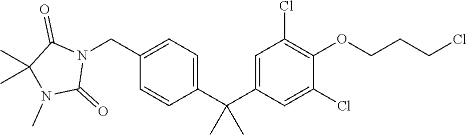 [SMILES: CN1C(=O)N(Cc2ccc(C(C)(C)c3cc(Cl)c(OCCCCl)c(Cl)c3)cc2)C(=O)C1(C)C] |
| B2 | 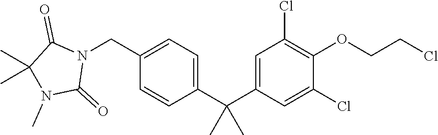 [SMILES: CN1C(=O)N(Cc2ccc(C(C)(C)c3cc(Cl)c(OCCCl)c(Cl)c3)cc2)C(=O)C1(C)C] |
| B3 | 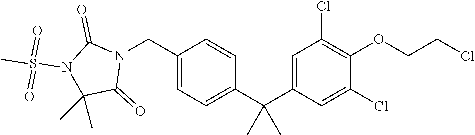 [SMILES: CC(C)(c1ccc(CN2C(=O)N(S(C)(=O)=O)C(C)(C)C2=O)cc1)c1cc(Cl)c(OCCCl)c(Cl)c1] |
| B4 | 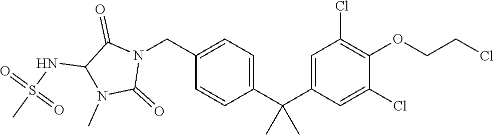 [SMILES: CN1C(=O)N(Cc2ccc(C(C)(C)c3cc(Cl)c(OCCCl)c(Cl)c3)cc2)C(=O)C1NS(C)(=O)=O] |
| B5 | 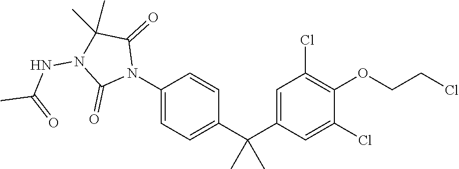 [SMILES: CC(=O)NN1C(=O)N(c2ccc(C(C)(C)c3cc(Cl)c(OCCCl)c(Cl)c3)cc2)C(=O)C1(C)C] |
| B6 | 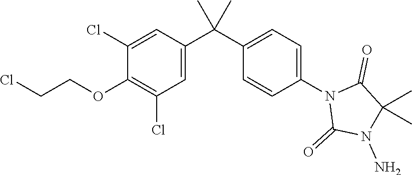 [SMILES: CC(C)(c1ccc(N2C(=O)N(N)C(C)(C)C2=O)cc1)c1cc(Cl)c(OCCCl)c(Cl)c1] |
| B7 | 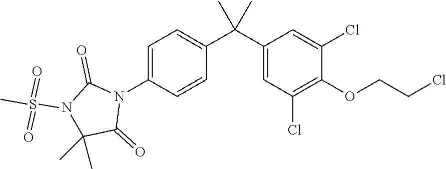 [SMILES: CC(C)(c1ccc(N2C(=O)N(S(C)(=O)=O)C(C)(C)C2=O)cc1)c1cc(Cl)c(OCCCl)c(Cl)c1] |
| B8 | 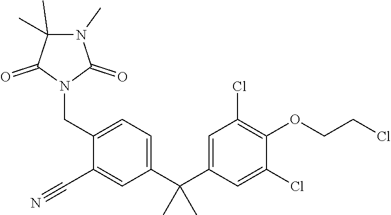 [SMILES: CN1C(=O)N(Cc2ccc(C(C)(C)c3cc(Cl)c(OCCCl)c(Cl)c3)cc2C#N)C(=O)C1(C)C] |
| B9 | 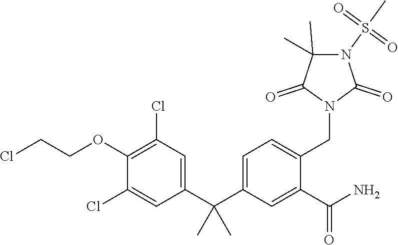 [SMILES: CC(C)(c1cc(Cl)c(OCCCl)c(Cl)c1)c1ccc(CN2C(=O)N(S(C)(=O)=O)C(C)(C)C2=O)c(C(N)=O)c1] |
| B10 | 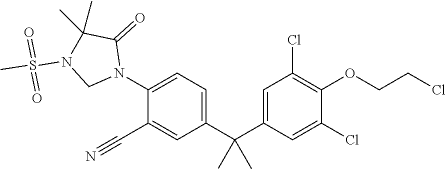 [SMILES: CC(C)(c1cc(Cl)c(OCCCl)c(Cl)c1)c1ccc(N2CN(S(C)(=O)=O)C(C)(C)C2=O)c(C#N)c1] |
| B11 | 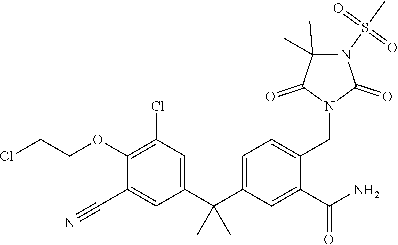 [SMILES: CC(C)(c1cc(Cl)c(OCCCl)c(C#N)c1)c1ccc(CN2C(=O)N(S(C)(=O)=O)C(C)(C)C2=O)c(C(N)=O)c1] |

In some embodiments, the the PTC is selected from:

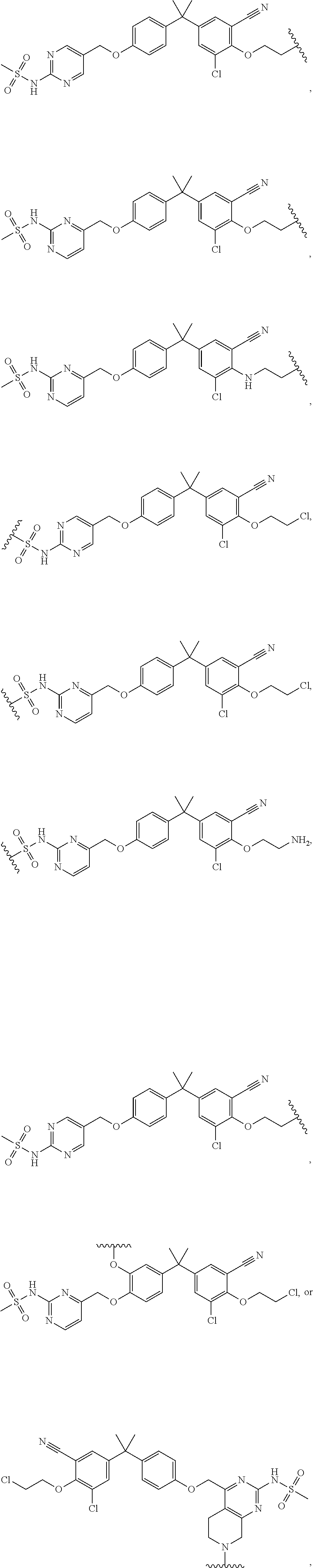

or a pharmaceutically acceptable salt, tautomer, stereoisomer or prodrug thereof.

In one embodiment, the present disclosure provides PTCs comprising the structure of formula (i):

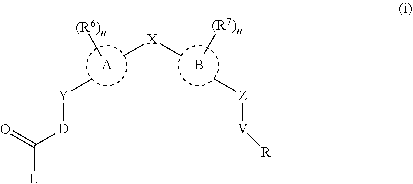

or a pharmaceutically acceptable salt, tautomer, stereoisomer or prodrug thereof, wherein:
A and B are each independently aryl or heteroaryl;
X is a bond, —(CR⁸R⁹)ₜ—, —O—, —C(═O)—, —S(O)ₙ—, —NR¹⁰—, —CONR¹⁰—, —NR¹⁰CO—, —SO₂NR¹⁰—, or —NR¹⁰SO₂—;
Y and Z are each independently a bond, —(CR⁸R⁹)ₜ—, —O—, —S(O)ₙ—, —NR¹⁰—, —CONR¹⁰—, —NR¹⁰CO—, —SO₂NR¹⁰—, or —NR¹⁰SO₂—;
V is a bond, optionally substituted —(CR¹¹R¹²)ₘ—, —C(═O)—, —N(R¹⁰)CO—, —CONR¹⁰—, or —NSO₂R¹⁰—;
R is —(CR^{4a}R^{4b})—(CR^{5a}R^{5b})—W or W;
W is hydrogen, halogen, optionally substituted alkylsulfonate, optionally substituted arylsufonate, —CF₃, —CF₂R¹⁰, —CN, —OR¹³, —NR¹³R¹⁴, optionally substituted —CONR¹³R¹⁴, optionally substituted carbocyclyl, optionally substituted heterocyclyl, optionally substituted aryl, or optionally substituted heteroaryl;
D is —(CR^{1a}R^{1b})_{q}—, —O—, or —NR¹⁰—;
L is —(CR^{2a}R^{2b})—R³ or -E-R³;
E is —(CR^{2a}R^{2b})_{g}—, —O—, —NR¹⁰—, or —NR¹⁰—(CR^{2a}R^{2b})_{g};
R^{1a}, R^{1b}, R^{2a}, and R^{2b} are each independently hydrogen, halogen, hydroxy, optionally substituted C₁₋₆ alkyl, optionally substituted C₂-C₆ alkenyl, optionally substituted C₂-C₆ alkynyl, optionally substituted C₁₋₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
or alternatively, R^{1a} and R^{1b} taken together form an optionally substituted carbocyclyl, optionally substituted heterocyclyl, optionally substituted aryl, or optionally substituted heteroaryl;
or alternatively, R^{2a} and R^{2b} taken together form a CO, optionally substituted carbocyclyl, optionally substituted heterocyclyl, optionally substituted aryl, or optionally substituted heteroaryl;
or alternatively, R^{1a}, R^{1b}, R^{2a} and R^{2b} taken together form an optionally substituted carbocyclyl, optionally substituted heterocyclyl, optionally substituted aryl, or optionally substituted heteroaryl;
R^{4a}, R^{4b}, R^{5a}, and R^{5b} are each independently hydrogen, halogen, hydroxy, optionally substituted C₁₋₆ alkyl, optionally substituted C₂-C₆ alkenyl, optionally substituted C₂-C₆ alkynyl, optionally substituted C₁₋₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
or alternatively, R^{4a} and R^{4b} taken together form a CO, optionally substituted carbocyclyl, optionally substituted heterocyclyl, optionally substituted aryl, or optionally substituted heteroaryl;
or alternatively, R^{4a}, R^{4b}, R^{5a} and R^{5b} taken together form an optionally substituted carbocyclyl, optionally substituted heterocyclyl, optionally substituted aryl, or optionally substituted heteroaryl;
R³ is absent, hydrogen, —CN, —CF₃, —OH, optionally substituted C₁-C₆ alkyl, optionally substituted C₂-C₆ alkenyl, optionally substituted C₂-C₆ alkynyl, optionally substituted —OR¹⁵, optionally substituted C₁-C₆ alkoxy, —NH₂, —NR¹⁶R¹⁷, —NR¹⁶COR¹⁸, —NR¹⁶S(O)ₚR¹⁸, —CONR¹⁴R¹⁵, —SONR¹⁴R¹⁵, —SO₂NR¹⁴R¹⁵, optionally substituted —S(O)ₚR¹⁸, —N3, optionally substituted carbocyclyl, optionally substituted heterocyclyl, optionally substituted aryl, or optionally substituted heteroaryl;
R^{2a}, R^{2b} and R³ taken together form an optionally substituted carbocyclyl, optionally substituted heterocyclyl, optionally substituted aryl, or optionally substituted heteroaryl;
R⁶ and R⁷ are each independently H, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —COOH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R⁸, R⁹, R¹¹ and R¹² are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
or alternatively, R⁸ and R⁹ taken together form an optionally substituted carbocyclyl or optionally substituted heterocyclyl;
or alternatively, R¹¹ and R¹², on a same carbon atom or a different carbon atom, taken together form an optionally substituted carbocyclyl, optionally substituted heterocyclyl, optionally substituted aryl, or optionally substituted heteroaryl;
R¹⁰ is hydrogen, halogen, optionally substituted C₁-C₆ alkyl, optionally substituted C₁-C₆ alkoxy, —CO(C₁-C₆ alkyl), optionally substituted C₁-C₆ alkylamino, optionally substituted carbocyclyl, optionally substituted heterocyclyl, optionally substituted aryl, or optionally substituted heteroaryl;
or alternatively, R^{2a} and R¹⁰ taken together form an optionally substituted heterocyclyl;
R¹³, R¹⁴, R¹⁵, R¹⁶, R¹⁷ and R¹⁸ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
or alternatively, R¹⁴ and R¹⁵ are taken together to form an optionally substituted heterocyclyl, or optionally substituted heteroaryl;
or alternatively, R¹⁶ and R¹⁷ are taken together to form an optionally substituted heterocyclyl, or optionally substituted heteroaryl;
m is 0, 1, 2, 3, or 4;
each n is independently 0, 1 or 2;
each p is independently 0, 1 or 2;
q is 0, 1 or 2;
each g is independently 0, 1, 2, 3, or 4; and
each t is independently 1 or 2.

In one embodiment of the PTCs of formula (i), the compound has the structure of formula (ii):

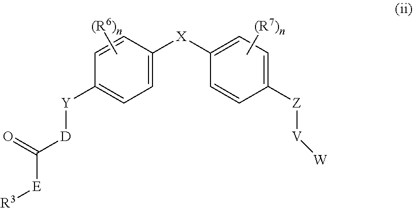

or a pharmaceutically acceptable salt, tautomer, stereoisomer or prodrug thereof, wherein:
X is a bond, —NR¹⁰—, or —(CR^{8a}R^{9a})ₜ—;
Y and Z are each independently a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—;
W is halogen, optionally substituted alkylsulfonate, optionally substituted arylsufonate, —NH₂, or —CF₃.
D is —NR¹⁰— and E is —(CR^{2a}R^{2b})_{g}—, —NR¹⁰—, or —NR¹⁰—(CR^{2a}R^{2b})_{g};
or alternatively, E is —NR¹⁰— or —NR¹⁰—(CR^{2a}R^{2b})_{g}—, and D is —(CR^{1a}R^{1b})_{q}— or —NR¹⁰—;
R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or (R^{1a} and R^{1b}) or (R^{2a} and R^{2b}) taken together form an oxo (═O), an optionally substituted carbocyclyl, or an optionally substituted heterocyclyl;
R³ is selected from hydrogen, —C₁-C₆ alkyl, —OR¹⁵, —SR¹⁸, —C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, —SOR¹⁸, or —SO₂R⁸;
R⁶ and R⁷ are each independently H, halogen, —CN, —CF₃, —OH, —COOH, —NH₂, —CONH₂, or C₁-C₃ alkyl;
R^{8a} and R^{9a} are each independently hydrogen, halogen, —OH, —NH₂, or C₁-C₃ alkyl;
R¹⁰ is each independently hydrogen, C₁-C₃ alkyl, C₂-C₃ alkenyl, C₂-C₃ alkynyl, —CO(C₁-C₃ alkyl);
R¹³, R¹⁴, R¹⁵, R¹⁶, R¹⁷ and R¹⁸ are each independently hydrogen, C₁-C₃ alkyl, C₂-C₃ alkenyl, or C₂-C₃ alkynyl; or R¹⁴ and R¹⁵ taken together form an optionally substituted 5- or 6-membered heterocyclyl;
each n is independently 0, 1 or 2;
q is 0, 1 or 2;
each g is independently 0, 1, 2, 3, or 4; and
each t is independently 1 or 2.

In one embodiment of the compounds of formula (i), the compound has the structure of formula (iii):

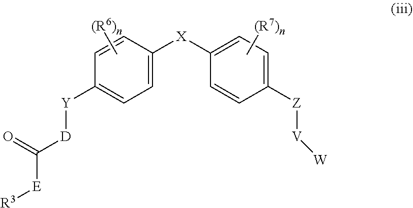

or a pharmaceutically acceptable salt, tautomer, stereoisomer or prodrug thereof, wherein:
X is a bond, —NR¹⁰—, or —(CR^{8a}R^{9a})ₜ—;
Y and Z are each independently a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—;
W is halogen, optionally substituted alkylsulfonate, optionally substituted arylsufonate, —NH₂ or —CF₃;
D is —NR¹⁰— and E is —(CR^{2a}R^{2b})_{gg}—;
or alternatively, E is —NR¹⁰— or —NR¹⁰—(CR^{2a}R^{2b})_{g}—, and D is —(CR^{1a}R^{1b})_{q}—;
R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or (R^{1a} and R^{1b}) or (R^{2a} and R^{2b}) taken together form an oxo (═O), an optionally substituted carbocyclyl, or an optionally substituted heterocyclyl;
R³ is selected from hydrogen, —C₁-C₆ alkyl, —OR¹⁵, —SR¹⁸, —C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, —SOR¹⁸, or —SO₂R¹⁸;
R⁶ and R⁷ are each independently H, halogen, —CN, —CF₃, —OH, —COOH, —NH₂, —CONH₂, or C₁-C₃ alkyl;
R^{8a} and R^{9a} are each independently hydrogen, halogen, —OH, —NH₂, or C₁-C₃ alkyl;
R¹⁰ is each independently hydrogen, C₁-C₃ alkyl, C₂-C₃ alkenyl, C₂-C₃ alkynyl, or —CO(C₁-C₃ alkyl);
R¹³, R¹⁴, R¹⁵, R¹⁶, R¹⁷ and R¹⁸ are each independently hydrogen, C₁-C₃ alkyl, C₂-C₃ alkenyl, or C₂-C₃ alkynyl; or R¹⁴ and R¹⁵ taken together form an optionally substituted 5- or 6-membered heterocyclyl;
m is 0, 1, 2, 3, or 4;
each n is independently 0, 1 or 2;
q is 1 or 2;
g is 0, 1, 2, 3, or 4;
gg is 1, 2, 3, or 4; and
t is 1 or 2.

In one embodiment of the PTCs of formula (i), the compound has the structure of formula (iv):

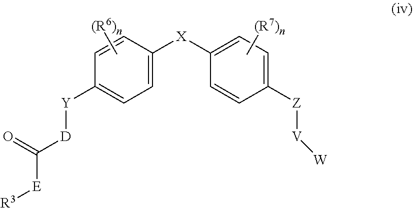

or a pharmaceutically acceptable salt, tautomer, stereoisomer or prodrug thereof, wherein:
X is a bond, —NR¹⁰—, or —(CR^{8a}R^{9a})ₜ—;
Y and Z are each independently a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—;
W is halogen, optionally substituted alkylsulfonate, optionally substituted arylsufonate, —CF₂R¹⁰, —NR¹³R¹⁴, optionally substituted carbocyclyl, optionally substituted heterocyclyl, optionally substituted aryl, or optionally substituted heteroaryl;
D is —(CR^{1a}R^{1b})_{q}—;
E is —(CR^{2a}R^{2b})_{g}—;
R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or (R^{1a} and R^{1b}) or (R^{2a} and R^{2b}) taken together form an oxo (═O), an optionally substituted carbocyclyl, or an optionally substituted heterocyclyl;
R³ is selected from hydrogen, —C₁-C₆ alkyl, —OR¹⁵, —SR¹⁸, —C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, —SOR¹⁸, or —SO₂R¹⁸;
R⁶ and R⁷ are each independently H, halogen, —CN, —CF₃, —OH, —COOH, —NH₂, —CONH₂, or C₁-C₃ alkyl;
R^{8a} and R^{9a} are each independently hydrogen, halogen, —OH, —NH₂, or C₁-C₃ alkyl; or R^{8a} and R^{9a} taken together form an optionally substituted carbocyclyl or optionally substituted heterocyclyl;
R¹⁰ is each independently hydrogen, C₁-C₃ alkyl, C₂-C₃ alkenyl, C₂-C₃ alkynyl, or —CO(C₁-C₃ alkyl);
R¹³, R¹⁴, R¹⁵, R¹⁶, R¹⁷ and R¹⁸ are each independently hydrogen, C₁-C₃ alkyl, C₂-C₃ alkenyl, or C₂-C₃ alkynyl; or R¹⁴ and R¹⁵ taken together form an optionally substituted 5- or 6-membered heterocyclyl;
m is 0, 1, 2, 3, or 4;
each n is independently 0, 1 or 2;
q is 0, 1 or 2;
g is 0, 1, 2, 3, or 4; and
t is 1 or 2.

In one embodiment of the PTCs of formula (i), R is W.

In one embodiment of the PTCs of formula (i), W is hydrogen, halogen, optionally substituted alkylsulfonate, optionally substituted arylsufonate, —CF₃, or —NR¹³R¹⁴. In one embodiment of the compounds of formula (i), W is halogen, optionally substituted alkylsulfonate, or optionally substituted arylsufonate. In one embodiment of the compounds of formula (i), W is halogen, mesylate, or tosylate.

In one embodiment of the PTCs of formula (i), W is hydrogen, halogen, —CF₃, or —NR¹³R¹⁴. In one embodiment, W is hydrogen, halogen, —CF₃, or —NH₂. In some embodiments, W is aryl, optionally substituted with halogen, C₁-C₃ alkyl, —CN, —CF₃, —OH, C₁-C₃ alkoxy, —NR¹³R¹⁴, or —SO₂R¹⁶. In another embodiment, W is a phenyl, optionally substituted with halogen, C₁-C₃ alkyl, —CN, —CF₃, —OH, or C₁-C₃ alkoxy.

In one embodiment of the PTCs of formula (i)-(iii), W is hydrogen, halogen, —CF₃, or —NH₂. In one embodiment, W is a halogen. In one embodiment, W is Cl, Br, I, or F. In other embodiments, W is Cl.

In one embodiment of the PTCs of formula (i)-(iii), W is halogen, optionally substituted alkylsufonate, or optionally substituted arylsulfonate.

In one embodiment of the PTCs of formula (i), L is -E-R³.

In one embodiment of the PTCs of formula (i)-(iv), R³ is selected from hydrogen, optionally substituted C₁-C₆ alkyl, optionally substituted —OR¹⁵, optionally substituted —SR¹⁸, optionally substituted C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, optionally substituted —SOR¹⁸, or optionally substituted —SO₂R¹⁸. In one embodiment, R³ is selected from hydrogen, —C₁-C₃ alkyl, —NR¹⁶SO(C₁-C₃ alkyl), —NR¹⁶SO₂(C₁-C₃ alkyl), —SONR¹⁴R¹⁵, —SO₂NR¹⁴R¹⁵, —SOR¹⁸, or —SO₂R¹⁸. In other embodiments, R³ is selected from —NHSO₂(C₁-C₃ alkyl), —NCH₃SO₂(C₁-C₃ alkyl), or —SO₂(C₁-C₃ alkyl).

In one embodiment of the PTCs of formula (i)-(iv), R³ is —NR¹⁶R¹⁷, —NR¹⁶COR¹⁸, —NR¹⁶S(O)ₚR¹⁸, —S(O)ₚR¹⁸, optionally substituted carbocyclyl, optionally substituted heterocyclyl, optionally substituted aryl, or optionally substituted heteroaryl. In other embodiments, R³ is —NR¹⁶R¹⁷. In one embodiment, R³ is —NR¹⁶S(O)ₚR¹⁸ or —S(O)ₚR¹⁸.

In one embodiment of the PTCs of formula (i)-(iv), R³ is hydrogen, —CN, —CF₃, —OH, optionally substituted C₁-C₆ alkyl, optionally substituted C₂-C₆ alkenyl, optionally substituted C₂-C₆ alkynyl, optionally substituted —OR¹⁵, optionally substituted C₁-C₆ alkoxy, —NH₂, —NR¹⁶R¹⁷, —NR¹⁶COR¹⁸, —NR¹⁶S(O)ₚR¹⁸, —CONR¹⁴R¹⁵, —SONR¹⁴R¹⁵, —SO₂NR¹⁴R¹⁵, optionally substituted —S(O)ₚR¹⁸, —N₃, optionally substituted carbocyclyl, optionally substituted heterocyclyl, optionally substituted aryl, or optionally substituted heteroaryl. In some embodiments, R³ is hydrogen, —CN, —CF₃, —OH, optionally substituted C₁-C₃ alkyl, optionally substituted C₂-C₃ alkenyl, optionally substituted C₂-C₃ alkynyl, optionally substituted —OR¹⁵, optionally substituted C₁-C₃ alkoxy, —NH₂, —NR¹⁶R¹⁷, —NR¹⁶COR¹⁸, —NR¹⁶S(O)ₚR¹⁸, —CONR¹⁴R¹⁵, —SONR¹⁴R¹⁵, —SO₂NR¹⁴R¹⁵, optionally substituted —S(O)ₚR¹⁸, —N₃, optionally substituted 3- to 7-membered carbocyclyl, optionally substituted 3- to 7-membered heterocyclyl, optionally substituted 6- to 12-membered aryl, or optionally substituted 5- to 12-membered heteroaryl. In other embodiments, R³ is hydrogen, —CN, —CF₃, —OH, optionally substituted C₁-C₃ alkyl, optionally substituted C₂-C₃ alkenyl, optionally substituted C₂-C₃ alkynyl, optionally substituted —OR¹⁵, optionally substituted C₁-C₃ alkoxy, —NH₂, —NR¹⁶R¹⁷, —NR¹⁶COR¹⁸, —NR¹⁶S(O)ₚR¹⁸, —CONR¹⁴R¹⁵, —SONR¹⁴R¹⁵, —SO₂NR¹⁴R¹⁵, optionally substituted —S(O)ₚR¹⁸, or -N3. In one embodiment, R³ is optionally substituted 3- to 7-membered carbocyclyl, optionally substituted 3- to 7-membered heterocyclyl, optionally substituted 6-membered aryl, or optionally substituted 5- to 6-membered heteroaryl.

In one embodiment of the PTCs of formula (i)-(iv), R³ is selected from hydrogen, C₁-C₆ alkyl, C₁-C₆ alkoxy, —NHSO₂(C₁-C₆ alkyl), —NCH₃SO₂(C₁-C₆ alkyl), —SO₂(C₁-C₆ alkyl), —NHCO(C₁-C₆ alkyl), or —N(C₁-C₆ alkyl)CO(C₁-C₆ alkyl). In another embodiment, R³ is hydrogen, C₁-C₃ alkyl, C₁-C₃ alkoxy, —NHSO₂(C₁-C₃ alkyl), —NCH₃SO₂(C₁-C₃ alkyl), —SO₂(C₁-C₃ alkyl), —NHCO(C₁-C₃ alkyl), or —N(C₁-C₃ alkyl)CO(C₁-C₃ alkyl). In other embodiments, R³ is selected from —NHSO₂(C₁-C₃ alkyl), —NCH₃SO₂(C₁-C₃ alkyl), or —SO₂(C₁-C₃ alkyl). In one embodiment, R³ is selected from hydrogen, C₁-C₃ alkyl, C₂-C₃ alkenyl, C₂-C₃ alkynyl, C₁-C₃ alkoxy, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl). In one embodiment, R³ is selected from hydrogen, methyl, ethyl, propyl, isopropyl, methoxy, —SO₂CH₃, —NHSO₂CH₃, or —N(CH₃)SO₂CH₃. In one embodiment, R³ is selected from —SO₂CH₃, —NHSO₂CH₃, or —N(CH₃)SO₂CH₃.

In one embodiment of the PTCs of formula (i)-(iv), R³ is C₁-C₆ alkyl.

In one embodiment of the PTCs of formula (i)-(iv), R³ is —NR¹⁶R¹⁷, wherein R¹⁶ and R¹⁷ are taken together to form an 3- to 7-membered optionally substituted heterocyclyl. In one embodiment, R³ is —NR¹⁶R¹⁷, wherein R¹⁶ and R¹⁷ are taken together to form a 6-membered optionally substituted heterocycle. In one embodiment, R³ is —NR¹⁶R¹⁷, wherein R¹⁶ and R¹⁷ are taken together to form a 6-membered optionally substituted heterocycle. In one embodiment, R³ is —NR¹⁶R¹⁷, wherein R¹⁶ and R¹⁷ are taken together to form an optionally substituted piperizine. In one embodiment, R³ is —NR¹⁶R¹⁷, wherein R¹⁶ and R¹⁷ are taken together to form a piperizine, optionally substituted with —SO₂CH₃, —NHSO₂CH₃, or —N(CH₃)SO₂CH₃. In one embodiment, R³ is

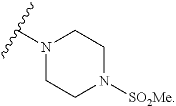 [SMILES: *N1CCN(S(C)(=O)=O)CC1]

In one embodiment of the PTCs of formula (i)-(iv), X is a bond, —NR¹⁰—, or —(CR^{8a}R^{9a})ₜ—. In one embodiment, X is a bond. In other embodiments, X is —(CR⁸R⁹)ₜ— or —NR¹⁰—. In another embodiment, X is —NR¹⁰—.

In one embodiment of the PTCs of formula (i)-(iv), X is —NR¹⁰—, wherein R¹⁰ is hydrogen or optionally substituted C₁-C₆ alkyl. In another embodiment, R¹⁰ is hydrogen. In some embodiments, X is —NR¹⁰—, wherein R¹⁰ is methyl. In one embodiment, X is —NR¹⁰—, wherein R¹⁰ is H, C₁-C₆ alkyl, or —CO(C₁-C₆ alkyl). In one embodiment, X is —NR¹⁰—, wherein R¹⁰ is H, C₁-C₆ alkyl, or —CO(C₁-C₆ alkyl). In one embodiment, X is —NR¹⁰—, wherein R¹⁰ is H, C₁-C₃ alkyl, or —CO(C₁-C₃ alkyl).

In one embodiment of the PTCs of formula (i)-(iv), X is —(CR⁸R⁹)ₜ—. In one embodiment, X is —(CR⁸R⁹)—, wherein R⁸ and R⁹ are each selected from H, halogen, —OH, or C₁-C₆ alkyl. In one embodiment, X is a bond or —(CR^{8a}R^{9a})ₜ—, wherein R^{8a} and R^{9a} are each selected from H, halogen, —OH, or C₁-C₆ alkyl and t is 1, or 2. In some embodiments, X is —(CR⁸R⁹)ₜ—, wherein each R⁸ and R⁹ are independently hydrogen or optionally substituted C₁-C₆ alkyl. In some embodiments, X is —(CR⁸R⁹)ₜ—, wherein each R⁸ and R⁹ are hydrogen. In some embodiments, X is —(CR⁸R⁹)ₜ—, wherein each R⁸ and R⁹ are methyl. In some embodiments, X is —(CR⁸R⁹)ₜ—, wherein each R⁸ and R⁹ are H, C₁-C₆ alkyl, —OH, or —NH₂.

In one embodiment of the PTCs of formula (i)-(iv), X is a bond or —(CR^{8a}R^{9a})ₜ—, wherein t is 1, or 2.

In one embodiment of the PTCs of formula (i)-(iv), the instance of t when X is —(CR⁸R⁹)ₜ— is 1. In other embodiments, the instance of t when X is —(CR⁸R⁹)ₜ-t is 2.

In one embodiment of the PTCs of formula (i)-(iv), X—(CR^{8a}R^{9a})ₜ—, wherein R^{8a} and R^{9a} taken together form an optionally substituted carbocyclyl or optionally substituted heterocyclyl. In one embodiment, X—(CR^{8a}R^{9a})ₜ—, wherein R^{8a} and R^{9a} taken together form an optionally substituted 3- to 6-membered carbocyclyl or an optionally substituted 3- to 6-membered heterocyclyl containing one heteroatom selected from O, S, or N. In one embodiment, X—(CR^{8a}R^{9a})ₜ—, wherein R^{8a} and R^{9a} taken together form a 3- to 6-membered carbocyclyl or a 3- to 6-membered heterocyclyl containing one heteroatom selected from O, S, or N. In one embodiment, X—(CR^{8a}R^{9a})ₜ—, wherein R^{8a} and R^{9a} taken together form a 4-membered heterocyclyl containing one O.

In one embodiment of the PTCs of formula (i)-(iv), X is a bond, —NR¹⁰—, or —(CR^{8a}R^{9a})ₜ—. In one embodiment, X is a bond, —CH₂—, —C(CH₃)₂—, —CH₂CH₂—, —NH—, —N(CH₃)—, —N(iPr)-, or —N(COCH₃)—. In other embodiments, X is a bond, —NH—, —CH₂—, —C(CH₃)₂—, or —CH₂CH₂—. In other embodiments, X is a bond, —NH—, —N(COCH₃)—, —N(C₁-C₃ alkyl)-, —CH₂—, —CH(CH₃)—, —C(CH₃)₂—, —CH₂CH₂—, —CH(OH)—, —CHF—, or —CHF₂—. In other embodiments, X is a bond, —CH₂—, —C(CH₃)₂—, —CH₂CH₂—, —NH—, —N(CH₃)—, —N(iPr)-, or —N(COCH₃)—.

In one embodiment of the PTCs of formula (i)-(iii), X is —(CR^{8a}OH)— or (CR^{8a}NH₂)—.

In one embodiment of the PTCs of formula (i), Y is —(CR⁸R⁹)ₜ—, —O—, or —NR¹⁰—. In other embodiments, Y is —O—. In another embodiment, Y is —(CR⁸R⁹)ₜ—. In another embodiment, Y is —(CR⁸R⁹)ₜ—, wherein each R⁸ and R⁹ are hydrogen.

In one embodiment of the PTCs of formula (i), the instance of t when Y is —(CR⁸R⁹)ₜ— is 1.

In one embodiment of the PTCs of formula (i), Y is —NR¹⁰—. In another embodiment, Y is —NR¹⁰—, wherein R¹⁰ is hydrogen or optionally substituted C₁-C₆ alkyl. In another embodiment, Y is —NR¹⁰—, R¹⁰ is hydrogen. In another embodiment, Y is —NR¹⁰—, R¹⁰ is methyl.

In one embodiment of the compounds of formula (i)-(iv), Y is a bond, —CH₂—, —C(CH₃)H—, —O—, —S—, —NH—, —NCH₃—, or —N(COCH₃)—. In one embodiment, Y is a —CH₂—, —C(CH₃)H—, —O—, —S—, —NH—, —NCH₃—, or —N(COCH₃)—. In one embodiment, Y is —CH₂—, —O—, —NH—, or —NCH₃—. In some embodiments, Y is a bond, —CH₂—, —O—, or —NCH₃—. In some embodiments, Y is a bond, —CH₂—, —O—, or —NH—. In some embodiments, Y is —O—.

In one embodiment of the PTCs of formula (i), Z is —(CR⁸R⁹)ₜ—, O, or NR¹⁰. In one embodiment, Z is O. In some embodiments, Z is —(CR⁸R⁹)ₜ—. In another embodiment, Z is —(CR⁸R⁹)ₜ—, wherein each R⁸ and R⁹ are hydrogen.

In one embodiment of the PTCs of formula (i), the instance of t when Z is —(CR⁸R⁹)ₜ— is 1.

In one embodiment of the PTCs of formula (i), Z is —NR¹⁰—. In some embodiments, Z is —NR¹⁰—, wherein R¹⁰ is hydrogen or optionally substituted C₁-C₆ alkyl. In some embodiments, Z is —NR¹⁰—, wherein R¹⁰ is hydrogen. In some embodiments, Z is —NR¹⁰—, wherein R¹⁰ is methyl.

In one embodiment of the PTCs of formula (i)-(iii), Z is a bond, —CH₂—, —C(CH₃)H—, —O—, —S—, —NH—, —NCH₃—, or —N(COCH₃)—. In one embodiment, Z is —CH₂—, —C(CH₃)H—, —O—, —S—, —NH—, —NCH₃—, or —N(COCH₃)—. In one embodiment, Z is —CH₂—, —O—, —NH—, —NCH₃—, or —N(COCH₃)—. In some embodiments, Z is a bond, —CH₂—, —O—, or —NCH₃—. In some embodiments, Z is a bond, —CH₂—, —O—, or —NH—. In some embodiments, Z is —O—.

In one embodiment of the PTCs of formula (i), V is a bond, —(CR¹¹R¹²)ₘ—, —C(═O)—, —N(R¹⁰)CO—, —CONR¹⁰—, or —NSO₂R¹⁰—. In one embodiment, V is a bond. In other embodiments, V is optionally substituted —C(R¹¹R¹²)ₘ—. In one embodiment, is optionally substituted —C(R¹¹R¹²)ₘ—, wherein each R¹¹ and R¹² are hydrogen. In some embodiments, V is —(CR¹¹R¹²)ₘ—.

In one embodiment of the compounds of formula (i), V is —(CR¹¹R¹²)ₘ—, wherein m is 1, 2, or 3. In some embodiments, V is —(CR¹¹R¹²)ₘ—, wherein R¹¹ and R¹² are each selected from H, halogen, —OH, or C₁-C₆ alkyl. In some embodiments, V is —(CR¹¹R¹²)ₘ—, wherein m is 1, 2, or 3. In some embodiments, V is —(CR¹¹R¹²)ₘ—, wherein R¹¹ and R¹² are each selected from H, halogen, —OH, or C₁-C₃ alkyl.

In one embodiment of the PTCs of formula (i)-(iv), V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—.

In one embodiment of the PTCs of formula (i), Z is a bond, —CH₂—, —C(CH₃)H—, —O—, —S—, —NH—, —NCH₃—, or —N(COCH₃)—; and V is —(CR¹¹R¹²)ₘ—.

In one embodiment of the PTCs of formula (i)-(iv), Z is a bond, —CH₂—, —C(CH₃)H—, —O—, —S—, —NH—, —NCH₃—, or —N(COCH₃)—; V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—; and W is halogen, —NH₂, or —CF₃.

In one embodiment of the PTCs of formula (i)-(iv), Z is a bond, —CH₂—, —C(CH₃)H—, —O—, —S—, —NH—, —NCH₃—, or —N(COCH₃)—; V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—; and W is halogen, —NH₂, or —CF₃.

In one embodiment of the PTCs of formula (i), m is 1 or 2. In some embodiments, m is 1.

In one embodiment of the PTCs of formula (i), each R⁸ and R⁹ are independently hydrogen or optionally substituted C₁-C₆ alkyl. In one embodiment, each R⁸ and R⁹ are H, C₁-C₆ alkyl, —OH, or —NH₂.

In one embodiment of the PTCs of formula (i), R^{1a} and R^{1b} are each hydrogen or optionally substituted C₁₋₆ alkyl. In other embodiments, R^{1a} and R^{1b} are each hydrogen.

In one embodiment of the PTCs of formula (i), R^{2a} and R^{2b} are each hydrogen or optionally substituted C₁₋₆ alkyl. In other embodiments, R^{2a} and R^{2b} are each hydrogen.

In one embodiment of the PTCs of formula (i)-(iv), R¹⁶ and R¹⁷ are taken together with the intervening atom to form an optionally substituted heterocyclyl, or optionally substituted heteroaryl. In some embodiments, R¹⁶ and R¹⁷ are taken together to form an optionally substituted heterocyclyl. In some embodiments, R¹⁶ and R¹⁷ are taken together to form an 3- to 7-membered optionally substituted heterocyclyl. In some embodiments, R¹⁶ and R¹⁷ are taken together to form an 3- to 7-membered optionally substituted heterocyclyl, comprising one or more heteroatoms selected from N, O, or S.

In one embodiment of the PTCs of formula (i)-(iv), R¹⁶ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁶ is hydrogen or optionally substituted C₁-C₆ alkyl. In some embodiments, R¹⁶ is hydrogen or C₁-C₆ alkyl. In some embodiments, R¹⁶ is hydrogen or C₁-C₃ alkyl.

In one embodiment of the PTCs of formula (i)-(iv), R¹⁷ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁷ is hydrogen or optionally substituted C₁-C₆ alkyl. In some embodiments, R¹⁷ is hydrogen or C₁-C₆ alkyl. In some embodiments, R¹⁷ is hydrogen or C₁-C₃ alkyl.

In one embodiment of the PTCs of formula (i)-(iv), R¹⁸ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁸ is hydrogen or optionally substituted C₁-C₆ alkyl. In some embodiments, R¹⁸ is hydrogen or C₁-C₆ alkyl. In some embodiments, R¹⁸ is hydrogen or C₁-C₃ alkyl.

In one embodiment of the PTCs of formula (i), R¹⁸ is C₁-C₆ alkyl; and p is 2.

In one embodiment of the PTCs of formula (i), R^{2a}, R^{2b} and R³ taken together with the intervening atom are optionally substituted carbocyclyl, optionally substituted heterocyclyl, optionally substituted aryl, or optionally substituted heteroaryl. In other embodiments, R^{2a}, R^{2b} and R³ taken together with the intervening atom are optionally substituted heteroaryl. In some embodiments, R^{2a}, R^{2b} and R³ taken together with the intervening atom are optionally substituted tetrazolyl, imidazolyl, 1,2,3-triazolyl, oxazole, pyrazinyl, pyrimidinyl, or 1,3,5-triazinyl.

In one embodiment of the PTCs of formula (i), R^{4a} is hydrogen, halogen, optionally substituted C₁₋₆ alkyl, or optionally substituted C₁₋₆ alkoxy. In some embodiments, R^{4a} is optionally substituted C₁₋₆ alkyl or hydrogen. In other embodiments, R^{4a} is hydroxy.

In one embodiment of the PTCs of formula (i), R^{4b} is hydrogen, halogen, optionally substituted C₁₋₆ alkyl, or optionally substituted C₁₋₆ alkoxy. In some embodiments, R^{4b} is hydrogen.

In one embodiment of the PTCs of formula (i), R^{5a} is hydrogen, halogen, optionally substituted C₁₋₆ alkyl, or optionally substituted C₁₋₆ alkoxy. In one embodiment, R^{5a} is hydrogen.

In one embodiment of the PTCs of formula (i), R^{5b} is hydrogen, halogen, optionally substituted C₁₋₆ alkyl, or optionally substituted C₁₋₆ alkoxy. In one embodiment, R^{5b} is hydrogen.

In one embodiment of the PTCs of formula (i), n is 1. In other embodiments, n is 2.

In one embodiment of the PTCs of formula (i), each occurrence of R⁶ and R⁷ is independently H, methyl, methoxy, CN, halogen, —OH, —NH₂, —COOH, or —CONH₂. In one embodiment of the PTCs of formula (i), each occurrence of R⁶ and R⁷ is independently H, methyl, methoxy, CN, F, Cl, Br, or I. In other embodiments, each occurrence of R⁶ and R⁷ is F, Cl, Br, or I. In one embodiment, each occurrence of R⁶ and R⁷ is Cl.

In one embodiment of the PTCs of formula (i), A and B are each independently 5- or 6-membered aryl or heteroaryl. In other embodiments, A and B are each independently selected from phenyl, pyridyl, pyrimidyl, or thiophene. In one embodiment, A and B are each phenyl.

In one embodiment of the PTCs of formula (i), A has a meta or para connectivity with X and Y. In one embodiment of the PTCs of formula (i), B has a meta or para connectivity with X and Z.

In one embodiment of the PTCs of formula (i)-(iv), D is —CH₂—, —CH(CH₃)—, —C(CH₃)₂—, or —CH₂CH₂—. In one embodiment, D is —(CH₂)₂—.

In one embodiment of the PTCs of formula (i), D is —(CR^{1a}R^{1b})_{q}—; E is —O—, —NR¹⁰—, or —NR¹—(CR^{2a}R^{2b})_{g}—; and q is 1 or 2.

In one embodiment of the PTCs of formula (i), D is —O— or —NR¹⁰—; E is —(CR^{2a}R^{2b})_{g}—; and g is 1, 2, 3, or 4.

In one embodiment of the PTCs of formula (i), D is —O— or —NR¹⁰—; and E is —O—, —NR¹⁰— or —NR¹⁰—(CR^{2a}R^{2b})_{g}—.

In one embodiment of the PTCs of formula (i), R⁶ and R⁷ are each independently halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶. In one embodiment, R⁶ and R⁷ are each independently halogen, —CN, —CF₃, —OH, optionally substituted C₁-C₃ alkyl, C₁-C₃ alkoxy, optionally substituted —(C₁-C₃ alkyl)-(C₁-C₃ alkoxy), optionally substituted —(C₁-C₃ alkyl)-OH, —NR¹³R¹⁴, —(C₁-C₃ alkyl)-NR¹³R¹⁴, —NR¹⁴SO₂R¹⁶, optionally substituted —(C₁-C₃ alkyl)NR¹⁴SO₂R¹⁶, —NR¹⁴COR¹⁶, optionally substituted —(C₁-C₃ alkyl)-NR¹⁴COR¹⁶, —CONR¹⁴R¹⁵, optionally substituted —(C₁-C₃ alkyl)-CONR¹⁴R¹⁵, —SO₂NR¹⁴R¹⁵, optionally substituted —(C₁-C₃ alkyl)-SO₂NR¹⁴R¹⁵, —SO₂R¹⁶, or optionally substituted —(C₁-C₃ alkyl)-SO₂R¹⁶. In one embodiment, R⁶ and R⁷ are each independently halogen, —CN, —CF₃, —OH, C₁-C₃ alkyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₃ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂R¹⁶, or —(C₁-C₃ alkyl)-SO₂R¹⁶. In one embodiment, R⁶ and R⁷ are each independently halogen, —CN, —CF₃, —OH, C₁-C₃ alkyl, or —CONR¹⁴R¹⁵. In some embodiments, R⁶ and R⁷ are each independently halogen, —CN, —CF₃, —OH, methyl, methoxy, or —CONH₂. In one embodiment, R⁶ and R⁷ are each independently Cl, —CN, —CF₃, —OH, methyl, methoxy, or —CONH₂. In one embodiment, R⁶ and R⁷ are each independently independently hydrogen, halogen, —OH, —NH₂, —CN, —CF₃, methyl, —COOH, or —CONH₂.

In one embodiment of the PTCs of formula (i), R⁶ and R⁷ are each independently optionally substituted carbocyclyl, optionally substituted heterocyclyl, optionally substituted aryl, or optionally substituted heteroaryl. In one embodiment, R⁶ and R⁷ are each independently 3- to 7-membered carbocyclyl, 3- to 7-membered heterocyclyl, phenyl, or 5- to 6-membered heteroaryl.

In one embodiment of the PTCs of formula (i)-(iv), R⁶ and R⁷ are each independently hydrogen, halogen, —OH, —NH₂, —CN, —CF₃, methyl, —COOH, or —CONH₂. In one embodiment, R⁶ and R⁷ is each independently halogen, —CN, —CF₃, —OH, methyl, or methoxy. In one embodiment, R⁶ and R⁷ are each independently halogen, —CN, —CF₃, —OH, or methyl. In one embodiment, R⁶ and R⁷ are each independently H, halogen, —CN, or methyl. In another embodiment, R⁶ and R⁷ is each independently Cl, —CN, —CF₃, —OH, methyl, or methoxy. In one embodiment, R⁶ and R⁷ is independently H, methyl, methoxy, CN, F, Cl, Br, or I. In one embodiment of the compounds of formula (i)-(iv), R⁶ and R⁷ is independently H, methyl, methoxy, CN, F, Cl, Br, I, ¹²³I or CF₃. In other embodiments, R⁶ and R⁷ is F, Cl, Br, or I. In one embodiment, each occurrence of R⁶ and R⁷ is Cl.

In one embodiment of the PTCs of formula (i)-(iv), R⁶ have one of the connectivity as shown below with respect to X and Y:

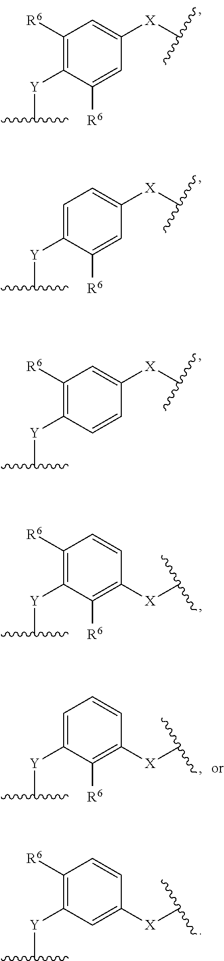

In one embodiment of the PTCs of formula (i)-(iv), R have one of the connectivity as shown below with respect to X and Z:

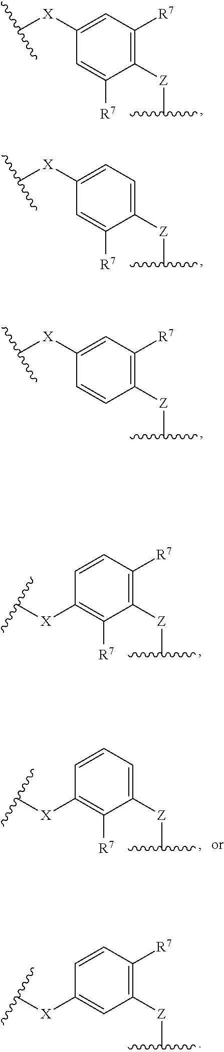

In one embodiment of the PTCs of formula (i)-(iv), n in —(R⁶)ₙ is 0, 1, or 2. In some embodiments, n is 0 or 1. In other embodiments, n is 0. In some embodiments, n is 1.

In one embodiment of the PTCs of formula (i)-(iv), n in —(R⁷)ₙ is 0, 1, or 2. In some embodiments, n is 0 or 1. In other embodiments, n is 0. In some embodiments, n is 1.

In one embodiment of the PTCs of formula (i)-(iv), the sum of n in —(R⁶)ₙ and —(R⁷)ₙ is 0, 1, 2, 3, or 4. In some embodiments, the sum of n in —(R⁶)ₙ and —(R⁷)ₙ is v 1, 2, 3, or 4. In some embodiments, the sum of n in —(R⁶)ₙ and —(R⁷)ₙ is 2 or 4. In some embodiments, the sum of n in —(R⁶)ₙ and —(R⁷)ₙ is 2.

In one embodiment of the PTCs of formula (i), R^{8b} and R^{9b} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl. In one embodiment, R¹¹ and R¹² are not —OH.

In one embodiment of the PTCs of formula (i), R¹¹ and R¹² are each independently hydrogen, halogen, —OH, or C₁-C₃ alkyl.

In one embodiment of the PTCs of formula (i), g is independently 0, 1, 2, or 3.

In one embodiment of the PTCs of formula (i), R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or (R^{1a} and Rib) or (R^{2a} and R^{2b}) taken together form an oxo (═O), an optionally substituted carbocyclyl, or an optionally substituted heterocyclyl.

In one embodiment of the PTCs of formula (i)-(iii), q is 0.

In one embodiment of the PTCs of formula (i)-(iii), E is —CH₂—, —CH(CH₃)—, —C(CH₃)₂—, —CH₂CH₂—, or —CH₂CH₂CH₂—.

In one embodiment of the PTCs of formula (i)-(iii), g is 0.

In one embodiment of the PTCs of formula (i)-(iii), at least one of Z and Y is —O—.

In one embodiment of the PTCs of formula (i) or (iv), Y is —O—, D is —(CR^{1a}R^{1b})_{q}—, L is —(CR^{2a}R^{2b})—R³, and R³ is —NR¹⁶S(O)ₚR¹⁸. In one embodiment, Y is —O—, D is —(CR^{1a}R^{1b})—, L is —(CR^{2a}R^{2b})—R³, and R³ is —NR¹⁶S(O)₂(C₁-C₃ alkyl). In one embodiment, Y is —O—, D is —CH₂—, —CH(CH₃)—, or —C(CH₃)₂—, L is —CH₂—R³, and R³ is —NHS(O)₂CH₃.

In one embodiment of the compounds of formula (i)-(iii), when E is —O—, R³ is hydrogen, —CF₃, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In other embodiments, when E is —O—, R³ is hydrogen, —CF₃, C₁-C₃ alkyl, C₂-C₃ alkenyl, C₂-C₃ alkynyl, carbocyclyl, heterocyclyl, aryl, or heteroaryl.

In one embodiment of the PTCs of formula (i)-(iv), at least one of Z and Y is —O—.

In one embodiment of the PTCs of formula (i)-(iv), -D-C(O)-E-R³ is

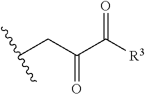

or its tautomeric form

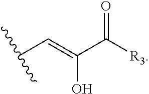

In one embodiment of the PTCs of formula (i)-(iv), —Y-D-C(O)-E-R³ is

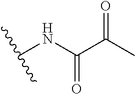 [SMILES: *NC(=O)C(C)=O]

or its tautomeric form

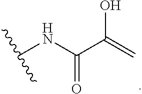 [SMILES: *NC(=O)C(=C)O]

In one embodiment of the PTCs of formula (i), -D-C(O)-E-R³ is

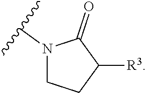

In one embodiment of the PTCs of formula (i)-(iv), R^{1a}, and R^{1b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, —(C₁-C₆ alkyl)-CONR¹⁴R¹⁵; or R^{1a} and R^{1b} taken together form an oxo (═O), an optionally substituted carbocyclyl, or an optionally substituted heterocyclyl. In some embodiments, R^{1a}, and R^{1b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R⁵. In one embodiment, R^{1a} and R^{1b} are each hydrogen or R^{1a} and R^{1b} taken together form an oxo (═O).

In one embodiment of the PTCs of formula (i)-(iv), R^{2a} and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or R^{2a} and R^{2b} taken together form an oxo (═O), an optionally substituted carbocyclyl, or an optionally substituted heterocyclyl. In some embodiments, R^{2a} and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵. In one embodiment, R^{2a} and R^{2b} are each hydrogen or R^{2a} and R^{2b} taken together form an oxo (═O).

In one embodiment of the PTCs of formula (ii)-(iii) R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl. In one embodiment, R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, or optionally substituted —(C₁-C₆ alkyl)-CONR¹⁴R¹⁵. In one embodiment, R^{8a} and R^{9a} hydrogen, halogen, —OH, or C₁-C₃ alkyl. In one embodiment, R^{8a} and R^{9a} hydrogen, halogen, —OH, or methyl. In one embodiment, R^{8a} and R^{9a} hydrogen, F, —OH, or methyl.

In one embodiment of the PTCs of formula (i)-(iv), R¹⁰ is hydrogen, halogen, optinally substituted C₁-C₆ alkyl, or optionally substituted C₁-C₆ alkoxy. In some embodiments, R¹⁰ is hydrogen, C₁-C₆ alkyl, or C₁-C₆ alkoxy. In some embodiments, R¹⁰ is hydrogen, C₁-C₃ alkyl, or C₁-C₃ alkoxy.

In one embodiment of the PTCs of formula (i), R^{2a} and R¹⁰ taken together form an optionally substituted heterocyclyl. In one embodiment, ^{2a} and R¹⁰ taken together form an optionally substituted 5- or 6-membered heterocyclyl.

In one embodiment of the PTCs of formula (i)-(iv), R¹³ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹³ is hydrogen or optionally substituted C₁-C₆ alkyl. In some embodiments, R¹³ is hydrogen or C₁-C₆ alkyl. In some embodiments, R¹³ is hydrogen or C₁-C₃ alkyl.

In one embodiment of the PTCs of formula (i)-(iv), R¹⁴ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁴ is hydrogen or optionally substituted C₁-C₆ alkyl. In some embodiments, R¹⁴ is hydrogen or C₁-C₆ alkyl. In some embodiments, R¹⁴ is hydrogen or C₁-C₃ alkyl.

In one embodiment of the PTCs of formula (i)-(iv), R¹⁵ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁵ is hydrogen or optionally substituted C₁-C₆ alkyl. In some embodiments, R¹⁵ is hydrogen or C₁-C₆ alkyl. In some embodiments, R¹⁵ is hydrogen or C₁-C₃ alkyl.

In one embodiment of the PTCs of formula (i)-(iv), R¹⁴ and R¹⁵ are taken together to form an optionally substituted heterocyclyl, or optionally substituted heteroaryl. In some embodiments, R¹⁴ and R¹⁵ are taken together to form an optionally substituted heterocyclyl. In some embodiments, R¹⁴ and R¹⁵ are taken together to form an 3- to 7-membered optionally substituted heterocyclyl. In some embodiments, R¹⁴ and R¹⁵ are taken together to form an 3- to 7-membered optionally substituted heterocyclyl, comprising one or more heteroatoms selected from N, O, or S.

In one embodiment of the PTCs of formula (i)-(iv), R¹¹ and R¹² are each independently hydrogen, halogen, —OH, or C₁-C₃ alkyl. In one embodiment, R¹¹ and R¹² are each independently hydrogen, halogen, or C₁-C₃ alkyl. In one embodiment, R¹¹ and R¹² are not —OH.

In one embodiment of the PTCs of formula (i)-(iv), g is independently 0, 1, 2, or 3. In one embodiment, g is 0. In another embodiment, g is 1, 2, or 3. In some embodiments, g is 1 or 2.

In one embodiment of the PTCs of formula (i), n is S(O)ₙ is 2. In another embodiment, n is 1 or 2. In some embodiments, n is 0.

In one embodiment of the PTCs of formula (i)-(iv), p is 2. In another embodiment, p is 1 or 2. In some embodiments, p is 0.

In one embodiment of the PTCs of formula (i)-(iv), q is 0. In another embodiment, q is 1. In one embodiment, q is 2.

In one embodiment of the PTCs of formula (i)-(iv), t is 1. In one embodiment, t is 2.

In one embodiment of the PTCs of formula (iii), gg is 1, 2, or 3. In some embodiments, gg is 1 or 2.

In one embodiment of the PTCs of formula (i)-(iii), Z and V are not both a bond or absent (e.g., m is 0 in —(CR¹¹R¹²)ₘ—).

In one embodiment of the PTCs of formula (i)-(iv), W can be halogen, optionally substituted alkyl sulfonate or optionally substituted aryl sulfonate. In one embodiment, W is halogen, tosylate or mesylate.

In one embodiment of the PTCs of formula (i)-(iv), X is —(CR⁸R⁹)— or —(CR^{8a}R^{9a})—, wherein R⁸, R⁹, R^{8a} and R^{9a} are each independently hydrogen, halogen, —OH, —NH₂, or —C₁-C₃ alkyl.

In one embodiment of the PTCs of formula (i)-(iv), R⁶ and R⁷ are each independently hydrogen, halogen, —OH, —NH₂, —CN, —CF₃, methyl, —COOH, or —CONH₂.

In one embodiment, the present disclosure provides PTCs as disclosed in Table C or a pharmaceutically acceptable salt, tautomer, stereoisomer or prodrug thereof. In one embodiment, the present disclosure provides a compound selected from Compounds AA1-AA98, or a pharmaceutically acceptable salt, tautomer, stereoisomer or prodrug thereof.

In one embodiment of the PTCs of formula (i)-(iv), the PTC is connected to the linker LI through R³, R⁶, R⁷, R¹⁰, R¹³, R¹⁴, R¹⁵, R¹⁶, or R¹⁷. In one embodiment of the PTCs of formula (i)-(iv), the PTC forms a covalent bond with the linker LI through standard organic chemistry protocols, such as substitution reactions and amino acid coupling reactions.

In one embodiment of the PTCs of formula (i)-(iv), a hydrogen (e.g., C—H, N—H, O—H, S—H), halogen, sulfonates (e.g., tosylate, mesylate), or any chemical group as defined in the formula (i)-(iv) is used to form the covalent bond between PTC and the linker LI. Thus, it can be understood that PTCs as disclosed herein as a neutral molecule is intended to be covalently bonded to the linker LI to form the protac molecule of formula (Q) by at least displacing one atom or one chemical group (e.g., H, halogen, OH, NH₂, OTs, OMs, etc) from the PTCs of formula (i)-(iv) to form the covalent bond with LI.

In one embodiment, PTC in formula Q is a compound of formula (i)-(iv), minus any functional group that was involved in making the PTC-LI bond.

The compounds disclosed in WO 2019/226991 can be useful PTCs for the present invention. The disclosure of WO 2019/226991 is incorporated by reference in its entirety for all purposes.

TABLE C
PTCs

| Compound | |
|---|---|
| ID | Structure |
| AA1 | 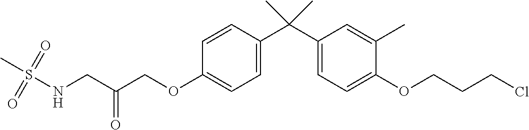 [SMILES: Cc1cc(C(C)(C)c2ccc(OCC(=O)CNS(C)(=O)=O)cc2)ccc1OCCCCl] |
| AA2 | 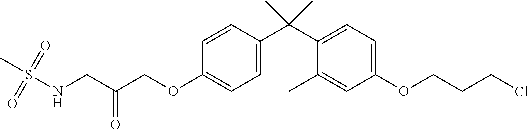 [SMILES: Cc1cc(OCCCCl)ccc1C(C)(C)c1ccc(OCC(=O)CNS(C)(=O)=O)cc1] |
| AA3 | 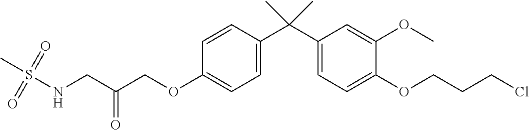 [SMILES: COc1cc(C(C)(C)c2ccc(OCC(=O)CNS(C)(=O)=O)cc2)ccc1OCCCCl] |
| AA4 | 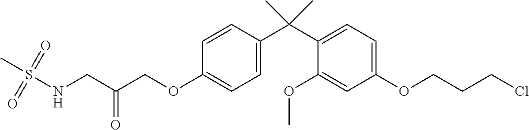 [SMILES: COc1cc(OCCCCl)ccc1C(C)(C)c1ccc(OCC(=O)CNS(C)(=O)=O)cc1] |
| AA5 | 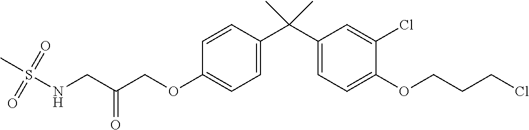 [SMILES: CC(C)(c1ccc(OCC(=O)CNS(C)(=O)=O)cc1)c1ccc(OCCCCl)c(Cl)c1] |
| AA6 | 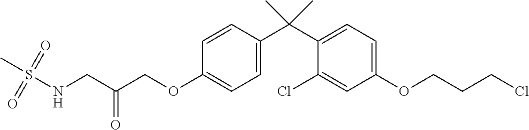 [SMILES: CC(C)(c1ccc(OCC(=O)CNS(C)(=O)=O)cc1)c1ccc(OCCCCl)cc1Cl] |
| AA7 | 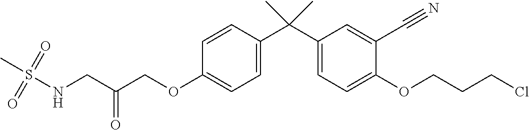 [SMILES: CC(C)(c1ccc(OCC(=O)CNS(C)(=O)=O)cc1)c1ccc(OCCCCl)c(C#N)c1] |
| AA8 | 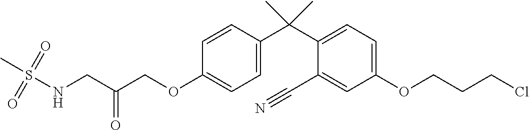 [SMILES: CC(C)(c1ccc(OCC(=O)CNS(C)(=O)=O)cc1)c1ccc(OCCCCl)cc1C#N] |
| AA9 | 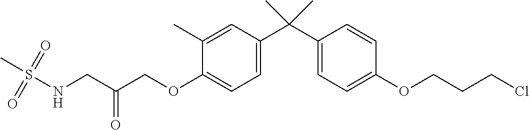 [SMILES: Cc1cc(C(C)(C)c2ccc(OCCCCl)cc2)ccc1OCC(=O)CNS(C)(=O)=O] |
| AA10 | 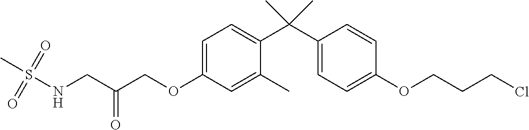 [SMILES: Cc1cc(OCC(=O)CNS(C)(=O)=O)ccc1C(C)(C)c1ccc(OCCCCl)cc1] |
| AA11 | 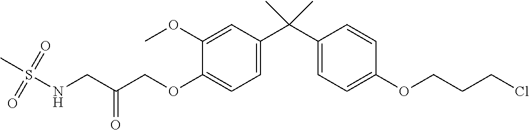 [SMILES: COc1cc(C(C)(C)c2ccc(OCCCCl)cc2)ccc1OCC(=O)CNS(C)(=O)=O] |
| AA12 | 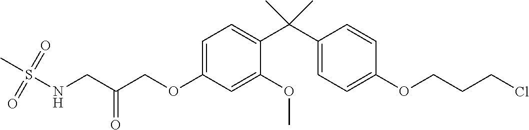 [SMILES: COc1cc(OCC(=O)CNS(C)(=O)=O)ccc1C(C)(C)c1ccc(OCCCCl)cc1] |
| AA13 | 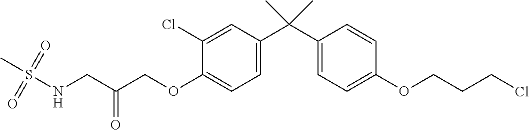 [SMILES: CC(C)(c1ccc(OCCCCl)cc1)c1ccc(OCC(=O)CNS(C)(=O)=O)c(Cl)c1] |
| AA14 | 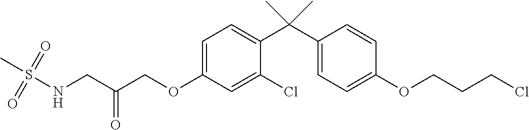 [SMILES: CC(C)(c1ccc(OCCCCl)cc1)c1ccc(OCC(=O)CNS(C)(=O)=O)cc1Cl] |
| AA15 | 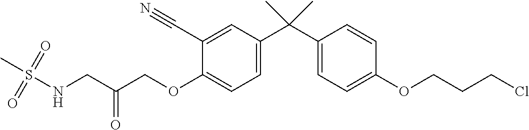 [SMILES: CC(C)(c1ccc(OCCCCl)cc1)c1ccc(OCC(=O)CNS(C)(=O)=O)c(C#N)c1] |
| AA16 | 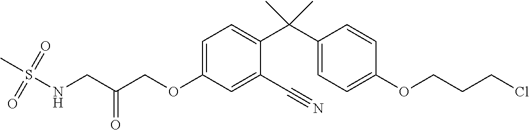 [SMILES: CC(C)(c1ccc(OCCCCl)cc1)c1ccc(OCC(=O)CNS(C)(=O)=O)cc1C#N] |
| AA17 | 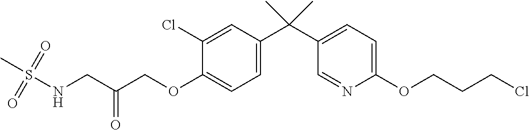 [SMILES: CC(C)(c1ccc(OCCCCl)nc1)c1ccc(OCC(=O)CNS(C)(=O)=O)c(Cl)c1] |
| AA18 | 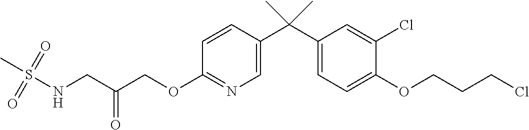 [SMILES: CC(C)(c1ccc(OCC(=O)CNS(C)(=O)=O)nc1)c1ccc(OCCCCl)c(Cl)c1] |
| AA19 | 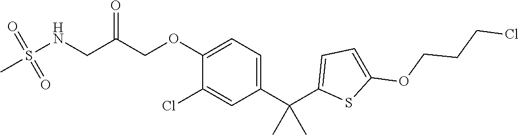 [SMILES: CC(C)(c1ccc(OCC(=O)CNS(C)(=O)=O)c(Cl)c1)c1ccc(OCCCCl)s1] |
| AA20 | 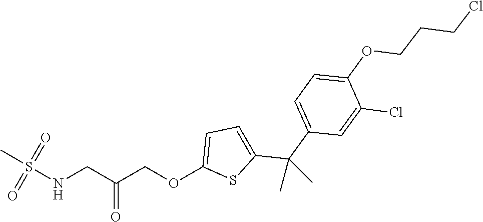 [SMILES: CC(C)(c1ccc(OCCCCl)c(Cl)c1)c1ccc(OCC(=O)CNS(C)(=O)=O)s1] |
| AA21 | 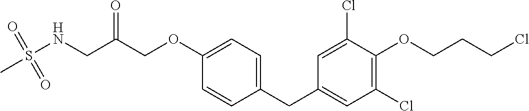 [SMILES: CS(=O)(=O)NCC(=O)COc1ccc(Cc2cc(Cl)c(OCCCCl)c(Cl)c2)cc1] |
| AA22 | 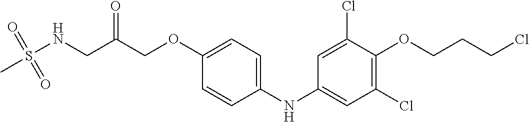 [SMILES: CS(=O)(=O)NCC(=O)COc1ccc(Nc2cc(Cl)c(OCCCCl)c(Cl)c2)cc1] |
| AA23 | 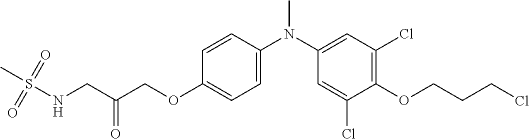 [SMILES: CN(c1ccc(OCC(=O)CNS(C)(=O)=O)cc1)c1cc(Cl)c(OCCCCl)c(Cl)c1] |
| AA24 | 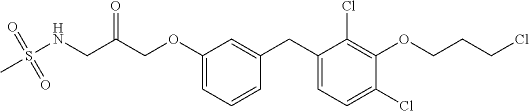 [SMILES: CS(=O)(=O)NCC(=O)COc1cccc(Cc2ccc(Cl)c(OCCCCl)c2Cl)c1] |
| AA25 | 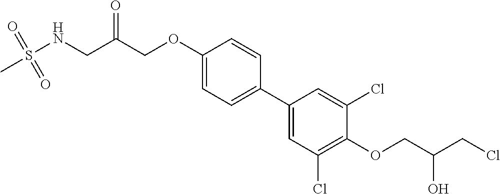 [SMILES: CS(=O)(=O)NCC(=O)COc1ccc(-c2cc(Cl)c(OCC(O)CCl)c(Cl)c2)cc1] |
| AA26 | 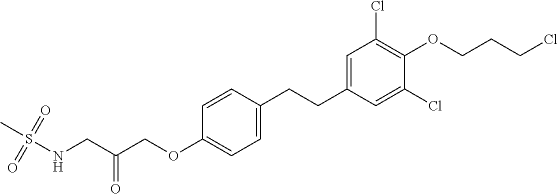 [SMILES: CS(=O)(=O)NCC(=O)COc1ccc(CCc2cc(Cl)c(OCCCCl)c(Cl)c2)cc1] |
| AA27 | 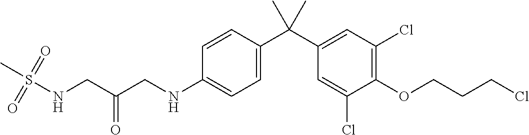 [SMILES: CC(C)(c1ccc(NCC(=O)CNS(C)(=O)=O)cc1)c1cc(Cl)c(OCCCCl)c(Cl)c1] |
| AA28 | 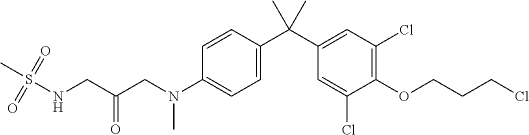 [SMILES: CN(CC(=O)CNS(C)(=O)=O)c1ccc(C(C)(C)c2cc(Cl)c(OCCCCl)c(Cl)c2)cc1] |
| AA29 | 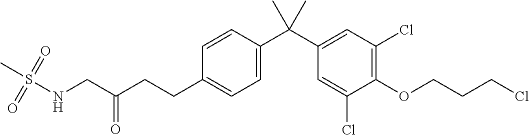 [SMILES: CC(C)(c1ccc(CCC(=O)CNS(C)(=O)=O)cc1)c1cc(Cl)c(OCCCCl)c(Cl)c1] |
| AA30 | 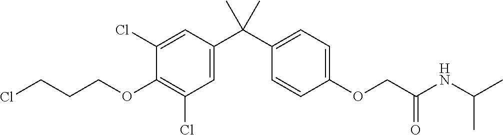 [SMILES: CC(C)NC(=O)COc1ccc(C(C)(C)c2cc(Cl)c(OCCCCl)c(Cl)c2)cc1] |
| AA31 | 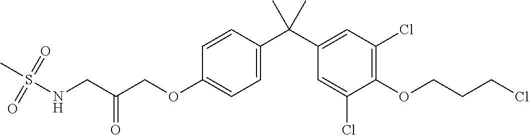 [SMILES: CC(C)(c1ccc(OCC(=O)CNS(C)(=O)=O)cc1)c1cc(Cl)c(OCCCCl)c(Cl)c1] |
| AA32 | 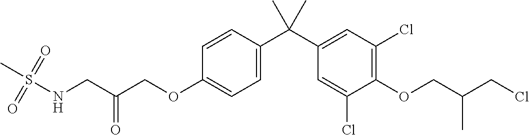 [SMILES: CC(CCl)COc1c(Cl)cc(C(C)(C)c2ccc(OCC(=O)CNS(C)(=O)=O)cc2)cc1Cl] |
| AA33 | 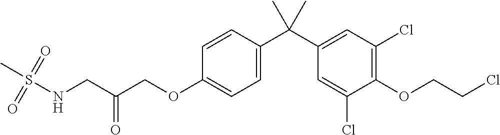 [SMILES: CC(C)(c1ccc(OCC(=O)CNS(C)(=O)=O)cc1)c1cc(Cl)c(OCCCl)c(Cl)c1] |
| AA34 | 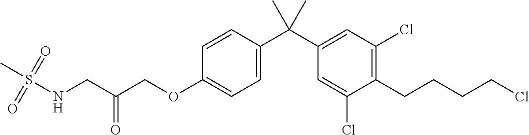 [SMILES: CC(C)(c1ccc(OCC(=O)CNS(C)(=O)=O)cc1)c1cc(Cl)c(CCCCCl)c(Cl)c1] |
| AA35 | 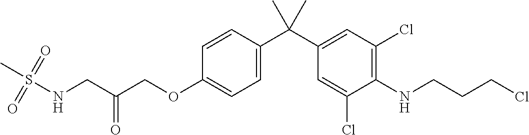 [SMILES: CC(C)(c1ccc(OCC(=O)CNS(C)(=O)=O)cc1)c1cc(Cl)c(NCCCCl)c(Cl)c1] |
| AA36 | 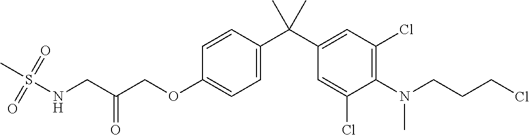 [SMILES: CN(CCCCl)c1c(Cl)cc(C(C)(C)c2ccc(OCC(=O)CNS(C)(=O)=O)cc2)cc1Cl] |
| AA37 | 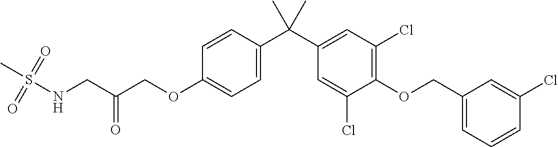 [SMILES: CC(C)(c1ccc(OCC(=O)CNS(C)(=O)=O)cc1)c1cc(Cl)c(OCc2cccc(Cl)c2)c(Cl)c1] |
| AA38 | 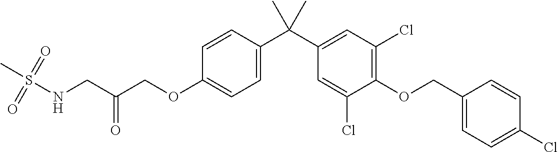 [SMILES: CC(C)(c1ccc(OCC(=O)CNS(C)(=O)=O)cc1)c1cc(Cl)c(OCc2ccc(Cl)cc2)c(Cl)c1] |
| AA39 | 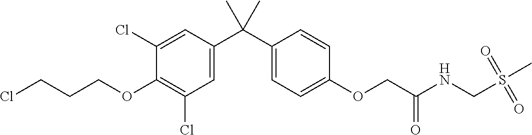 [SMILES: CC(C)(c1ccc(OCC(=O)NCS(C)(=O)=O)cc1)c1cc(Cl)c(OCCCCl)c(Cl)c1] |
| AA40 | 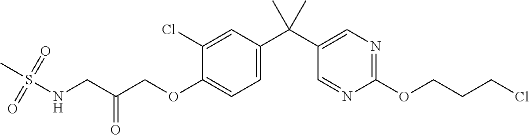 [SMILES: CC(C)(c1cnc(OCCCCl)nc1)c1ccc(OCC(=O)CNS(C)(=O)=O)c(Cl)c1] |
| AA41 | 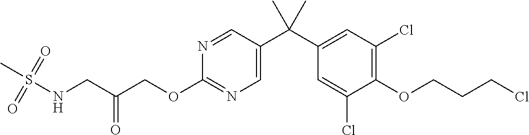 [SMILES: CC(C)(c1cnc(OCC(=O)CNS(C)(=O)=O)nc1)c1cc(Cl)c(OCCCCl)c(Cl)c1] |
| AA42 | 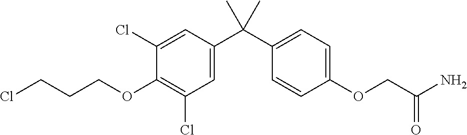 [SMILES: CC(C)(c1ccc(OCC(N)=O)cc1)c1cc(Cl)c(OCCCCl)c(Cl)c1] |
| AA43 | 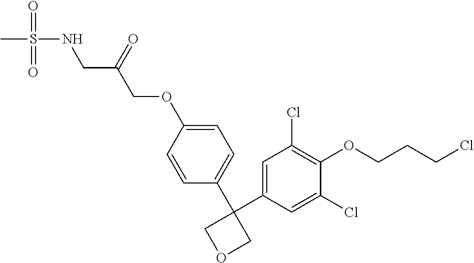 [SMILES: CS(=O)(=O)NCC(=O)COc1ccc(C2(c3cc(Cl)c(OCCCCl)c(Cl)c3)COC2)cc1] |
| AA44 | 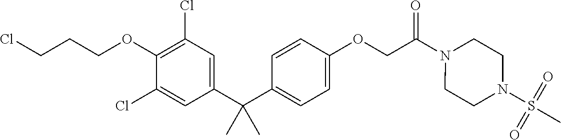 [SMILES: CC(C)(c1ccc(OCC(=O)N2CCN(S(C)(=O)=O)CC2)cc1)c1cc(Cl)c(OCCCCl)c(Cl)c1] |
| AA45 | 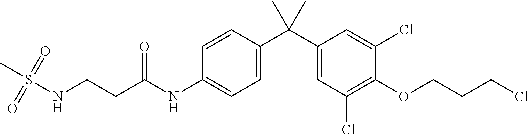 [SMILES: CC(C)(c1ccc(NC(=O)CCNS(C)(=O)=O)cc1)c1cc(Cl)c(OCCCCl)c(Cl)c1] |
| AA46 | 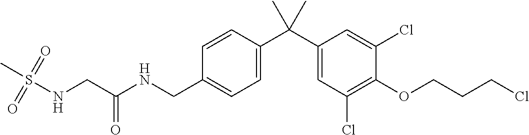 [SMILES: CC(C)(c1ccc(CNC(=O)CNS(C)(=O)=O)cc1)c1cc(Cl)c(OCCCCl)c(Cl)c1] |
| AA47 | 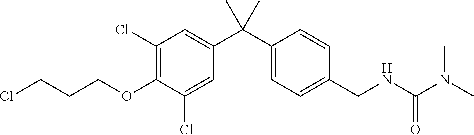 [SMILES: CN(C)C(=O)NCc1ccc(C(C)(C)c2cc(Cl)c(OCCCCl)c(Cl)c2)cc1] |
| AA48 | 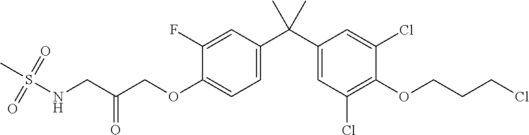 [SMILES: CC(C)(c1ccc(OCC(=O)CNS(C)(=O)=O)c(F)c1)c1cc(Cl)c(OCCCCl)c(Cl)c1] |
| AA49 | 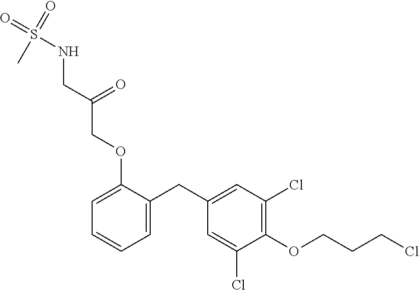 [SMILES: CS(=O)(=O)NCC(=O)COc1ccccc1Cc1cc(Cl)c(OCCCCl)c(Cl)c1] |
| AA50 | 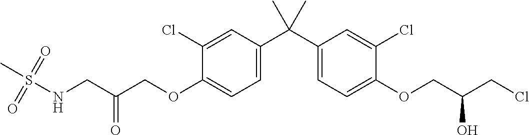 [SMILES: CC(C)(c1ccc(OCC(=O)CNS(C)(=O)=O)c(Cl)c1)c1ccc(OC[C@H](O)CCl)c(Cl)c1] |
| AA51 | 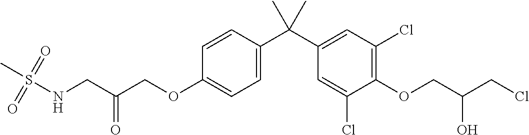 [SMILES: CC(C)(c1ccc(OCC(=O)CNS(C)(=O)=O)cc1)c1cc(Cl)c(OCC(O)CCl)c(Cl)c1] |
| AA51(S) | 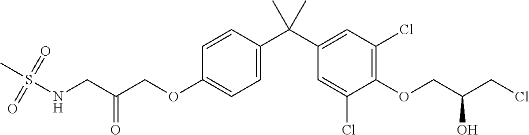 [SMILES: CC(C)(c1ccc(OCC(=O)CNS(C)(=O)=O)cc1)c1cc(Cl)c(OC[C@H](O)CCl)c(Cl)c1] |
| AA51(R) | 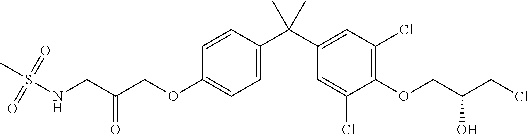 [SMILES: CC(C)(c1ccc(OCC(=O)CNS(C)(=O)=O)cc1)c1cc(Cl)c(OC[C@@H](O)CCl)c(Cl)c1] |
| AA52 | 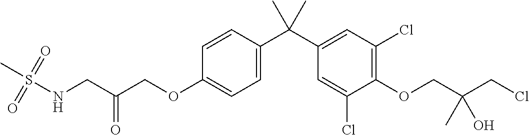 [SMILES: CC(O)(CCl)COc1c(Cl)cc(C(C)(C)c2ccc(OCC(=O)CNS(C)(=O)=O)cc2)cc1Cl] |
| AA52(S) | 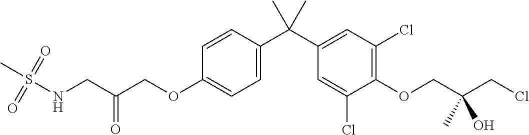 [SMILES: CC(C)(c1ccc(OCC(=O)CNS(C)(=O)=O)cc1)c1cc(Cl)c(OC[C@](C)(O)CCl)c(Cl)c1] |
| AA52(R) | 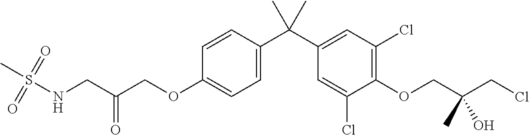 [SMILES: CC(C)(c1ccc(OCC(=O)CNS(C)(=O)=O)cc1)c1cc(Cl)c(OC[C@@](C)(O)CCl)c(Cl)c1] |
| AA53 | 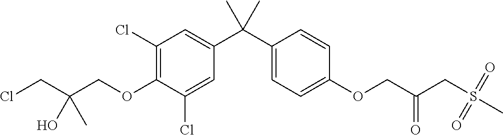 [SMILES: CC(O)(CCl)COc1c(Cl)cc(C(C)(C)c2ccc(OCC(=O)CS(C)(=O)=O)cc2)cc1Cl] |
| AA53(S) | 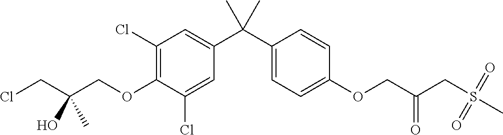 [SMILES: CC(C)(c1ccc(OCC(=O)CS(C)(=O)=O)cc1)c1cc(Cl)c(OC[C@@](C)(O)CCl)c(Cl)c1] |
| AA53(R) | 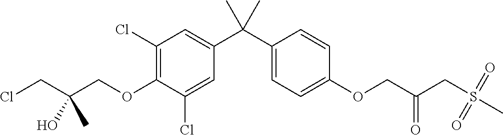 [SMILES: CC(C)(c1ccc(OCC(=O)CS(C)(=O)=O)cc1)c1cc(Cl)c(OC[C@](C)(O)CCl)c(Cl)c1] |
| AA54 | 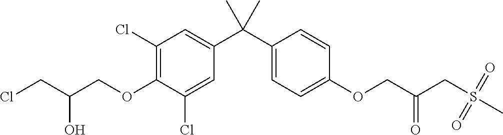 [SMILES: CC(C)(c1ccc(OCC(=O)CS(C)(=O)=O)cc1)c1cc(Cl)c(OCC(O)CCl)c(Cl)c1] |
| AA54(S) | 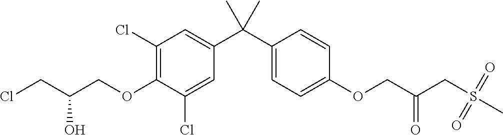 [SMILES: CC(C)(c1ccc(OCC(=O)CS(C)(=O)=O)cc1)c1cc(Cl)c(OC[C@H](O)CCl)c(Cl)c1] |
| AA54(R) | 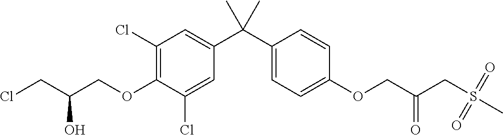 [SMILES: CC(C)(c1ccc(OCC(=O)CS(C)(=O)=O)cc1)c1cc(Cl)c(OC[C@@H](O)CCl)c(Cl)c1] |
| AA55 | 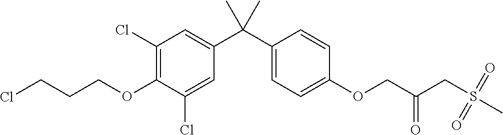 [SMILES: CC(C)(c1ccc(OCC(=O)CS(C)(=O)=O)cc1)c1cc(Cl)c(OCCCCl)c(Cl)c1] |
| AA56 | 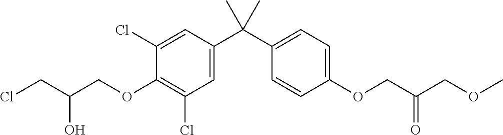 [SMILES: COCC(=O)COc1ccc(C(C)(C)c2cc(Cl)c(OCC(O)CCl)c(Cl)c2)cc1] |
| AA56(S) | 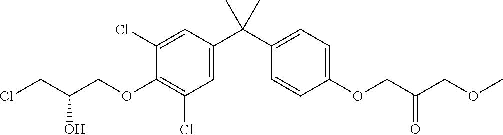 [SMILES: COCC(=O)COc1ccc(C(C)(C)c2cc(Cl)c(OC[C@H](O)CCl)c(Cl)c2)cc1] |
| AA56(R) | 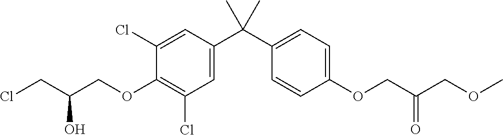 [SMILES: COCC(=O)COc1ccc(C(C)(C)c2cc(Cl)c(OC[C@@H](O)CCl)c(Cl)c2)cc1] |
| AA57 | 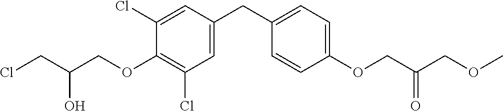 [SMILES: COCC(=O)COc1ccc(Cc2cc(Cl)c(OCC(O)CCl)c(Cl)c2)cc1] |
| AA57(S) | 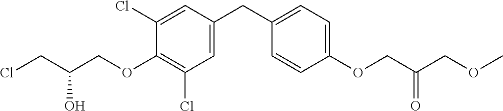 [SMILES: COCC(=O)COc1ccc(Cc2cc(Cl)c(OC[C@H](O)CCl)c(Cl)c2)cc1] |
| AA57(R) | 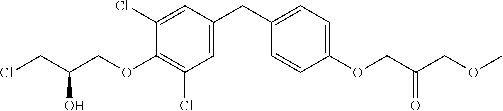 [SMILES: COCC(=O)COc1ccc(Cc2cc(Cl)c(OC[C@@H](O)CCl)c(Cl)c2)cc1] |
| AA58 | 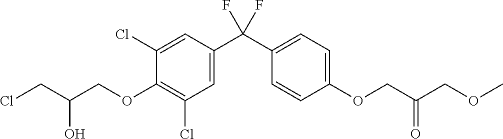 [SMILES: COCC(=O)COc1ccc(C(F)(F)c2cc(Cl)c(OCC(O)CCl)c(Cl)c2)cc1] |
| AA58(S) | 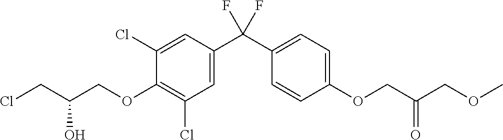 [SMILES: COCC(=O)COc1ccc(C(F)(F)c2cc(Cl)c(OC[C@H](O)CCl)c(Cl)c2)cc1] |
| AA58(R) | 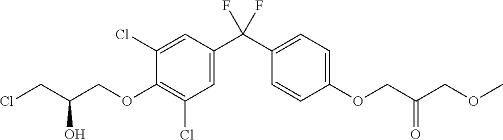 [SMILES: COCC(=O)COc1ccc(C(F)(F)c2cc(Cl)c(OC[C@@H](O)CCl)c(Cl)c2)cc1] |
| AA59 | 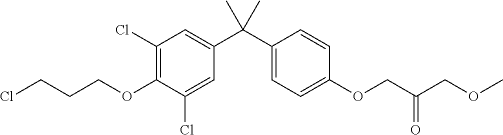 [SMILES: COCC(=O)COc1ccc(C(C)(C)c2cc(Cl)c(OCCCCl)c(Cl)c2)cc1] |
| AA60 | 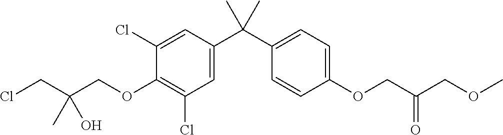 [SMILES: COCC(=O)COc1ccc(C(C)(C)c2cc(Cl)c(OCC(C)(O)CCl)c(Cl)c2)cc1] |
| AA60(S) | 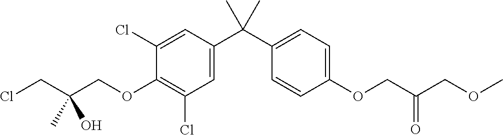 [SMILES: COCC(=O)COc1ccc(C(C)(C)c2cc(Cl)c(OC[C@@](C)(O)CCl)c(Cl)c2)cc1] |
| AA60(R) | 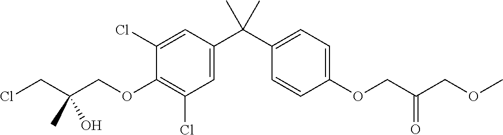 [SMILES: COCC(=O)COc1ccc(C(C)(C)c2cc(Cl)c(OC[C@](C)(O)CCl)c(Cl)c2)cc1] |
| AA61 | 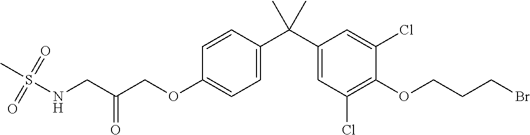 [SMILES: CC(C)(c1ccc(OCC(=O)CNS(C)(=O)=O)cc1)c1cc(Cl)c(OCCCBr)c(Cl)c1] |
| AA62 | 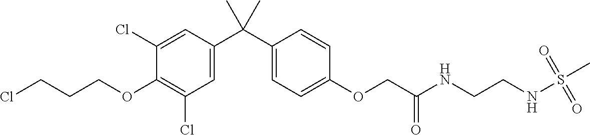 [SMILES: CC(C)(c1ccc(OCC(=O)NCCNS(C)(=O)=O)cc1)c1cc(Cl)c(OCCCCl)c(Cl)c1] |
| AA63 | 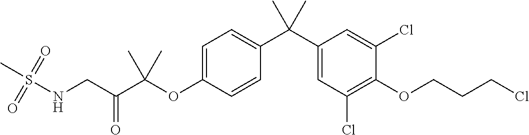 [SMILES: CC(C)(Oc1ccc(C(C)(C)c2cc(Cl)c(OCCCCl)c(Cl)c2)cc1)C(=O)CNS(C)(=O)=O] |
| AA64 | 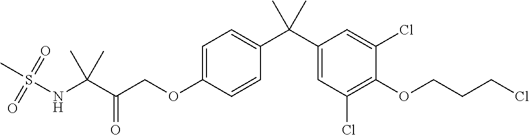 [SMILES: CC(C)(NS(C)(=O)=O)C(=O)COc1ccc(C(C)(C)c2cc(Cl)c(OCCCCl)c(Cl)c2)cc1] |
| AA65 | 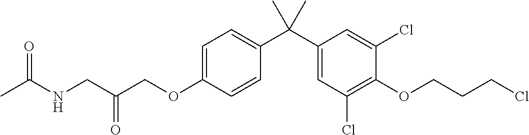 [SMILES: CC(=O)NCC(=O)COc1ccc(C(C)(C)c2cc(Cl)c(OCCCCl)c(Cl)c2)cc1] |
| AA66 | 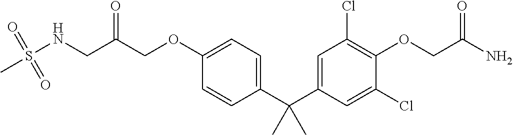 [SMILES: CC(C)(c1ccc(OCC(=O)CNS(C)(=O)=O)cc1)c1cc(Cl)c(OCC(N)=O)c(Cl)c1] |
| AA67 | 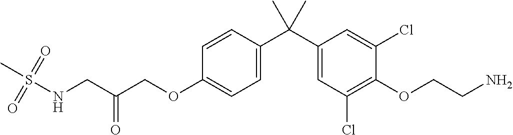 [SMILES: CC(C)(c1ccc(OCC(=O)CNS(C)(=O)=O)cc1)c1cc(Cl)c(OCCN)c(Cl)c1] |
| AA68 | 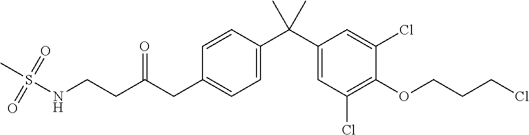 [SMILES: CC(C)(c1ccc(CC(=O)CCNS(C)(=O)=O)cc1)c1cc(Cl)c(OCCCCl)c(Cl)c1] |
| AA69 | 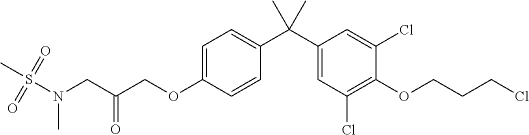 [SMILES: CN(CC(=O)COc1ccc(C(C)(C)c2cc(Cl)c(OCCCCl)c(Cl)c2)cc1)S(C)(=O)=O] |
| AA70 | 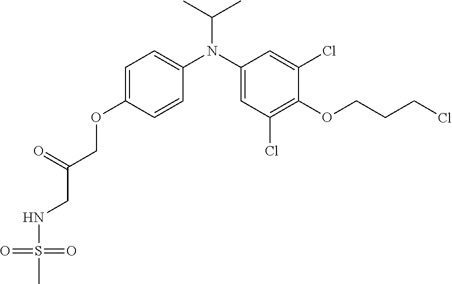 [SMILES: CC(C)N(c1ccc(OCC(=O)CNS(C)(=O)=O)cc1)c1cc(Cl)c(OCCCCl)c(Cl)c1] |
| AA71 | 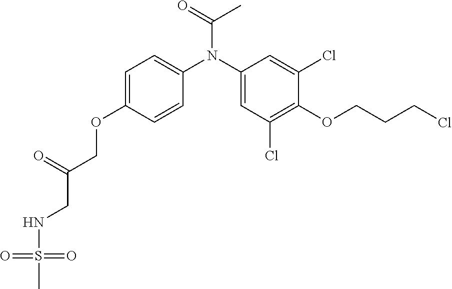 [SMILES: CC(=O)N(c1ccc(OCC(=O)CNS(C)(=O)=O)cc1)c1cc(Cl)c(OCCCCl)c(Cl)c1] |
| AA72 | 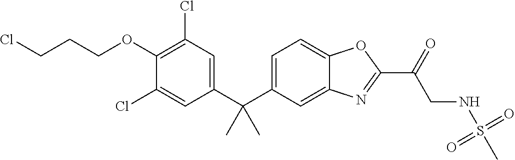 [SMILES: CC(C)(c1cc(Cl)c(OCCCCl)c(Cl)c1)c1ccc2oc(C(=O)CNS(C)(=O)=O)nc2c1] |
| AA73 | 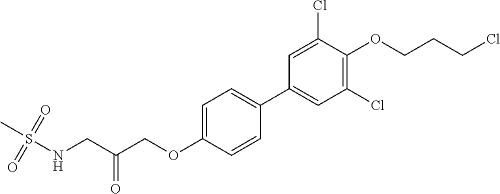 [SMILES: CS(=O)(=O)NCC(=O)COc1ccc(-c2cc(Cl)c(OCCCCl)c(Cl)c2)cc1] |
| AA74 | 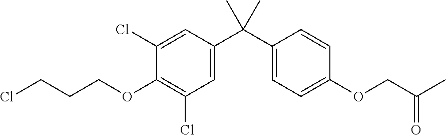 [SMILES: CC(=O)COc1ccc(C(C)(C)c2cc(Cl)c(OCCCCl)c(Cl)c2)cc1] |
| A75 | 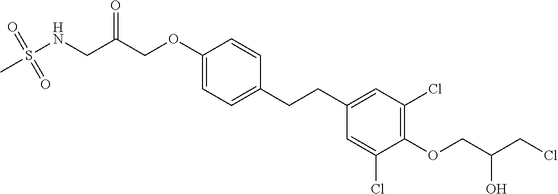 [SMILES: CS(=O)(=O)NCC(=O)COc1ccc(CCc2cc(Cl)c(OCC(O)CCl)c(Cl)c2)cc1] |
| AA76 | 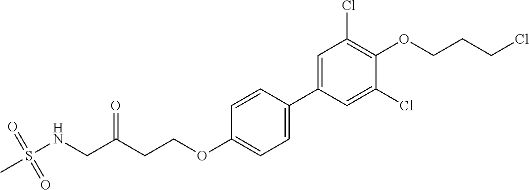 [SMILES: CS(=O)(=O)NCC(=O)CCOc1ccc(-c2cc(Cl)c(OCCCCl)c(Cl)c2)cc1] |
| AA77 | 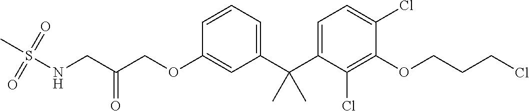 [SMILES: CC(C)(c1cccc(OCC(=O)CNS(C)(=O)=O)c1)c1ccc(Cl)c(OCCCCl)c1Cl] |
| A78 | 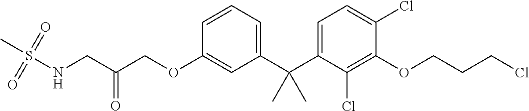 [SMILES: CC(C)(c1cccc(OCC(=O)CNS(C)(=O)=O)c1)c1ccc(Cl)c(OCCCCl)c1Cl] |
| AA79 | 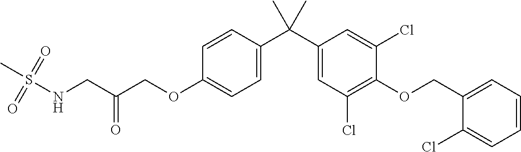 [SMILES: CC(C)(c1ccc(OCC(=O)CNS(C)(=O)=O)cc1)c1cc(Cl)c(OCc2ccccc2Cl)c(Cl)c1] |
| AA80 | 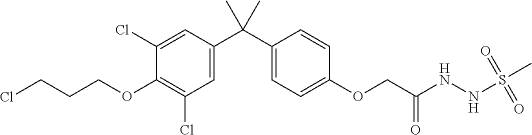 [SMILES: CC(C)(c1ccc(OCC(=O)NNS(C)(=O)=O)cc1)c1cc(Cl)c(OCCCCl)c(Cl)c1] |
| AA81 | 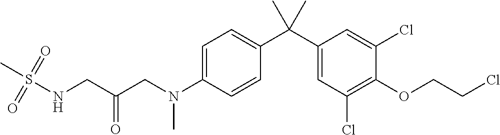 [SMILES: CN(CC(=O)CNS(C)(=O)=O)c1ccc(C(C)(C)c2cc(Cl)c(OCCCl)c(Cl)c2)cc1] |
| AA82 | 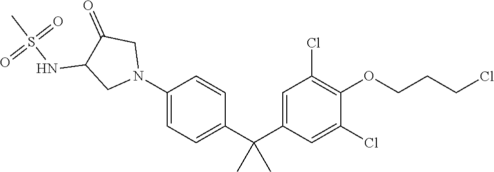 [SMILES: CC(C)(c1ccc(N2CC(=O)C(NS(C)(=O)=O)C2)cc1)c1cc(Cl)c(OCCCCl)c(Cl)c1] |
| AA83 | 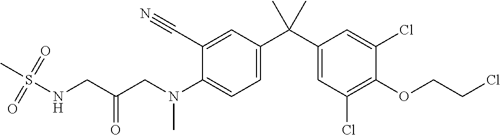 [SMILES: CN(CC(=O)CNS(C)(=O)=O)c1ccc(C(C)(C)c2cc(Cl)c(OCCCl)c(Cl)c2)cc1C#N] |
| AA84 | 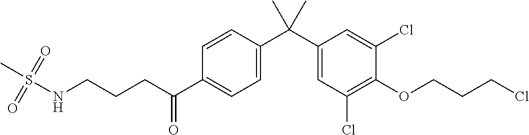 [SMILES: CC(C)(c1ccc(C(=O)CCCNS(C)(=O)=O)cc1)c1cc(Cl)c(OCCCCl)c(Cl)c1] |
| AA85 | 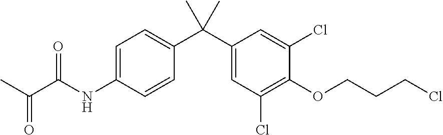 [SMILES: CC(=O)C(=O)Nc1ccc(C(C)(C)c2cc(Cl)c(OCCCCl)c(Cl)c2)cc1] |
| AA86 | 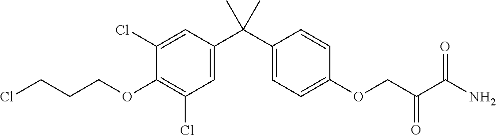 [SMILES: CC(C)(c1ccc(OCC(=O)C(N)=O)cc1)c1cc(Cl)c(OCCCCl)c(Cl)c1] |
| AA87 | 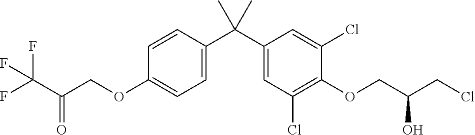 [SMILES: CC(C)(c1ccc(OCC(=O)C(F)(F)F)cc1)c1cc(Cl)c(OC[C@H](O)CCl)c(Cl)c1] |
| AA88 | 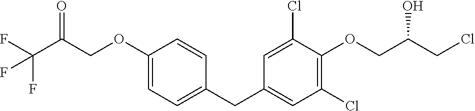 [SMILES: O=C(COc1ccc(Cc2cc(Cl)c(OC[C@H](O)CCl)c(Cl)c2)cc1)C(F)(F)F] |
| AA89 | 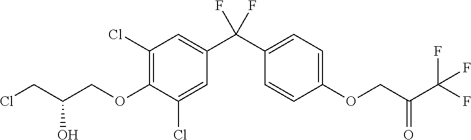 [SMILES: O=C(COc1ccc(C(F)(F)c2cc(Cl)c(OC[C@H](O)CCl)c(Cl)c2)cc1)C(F)(F)F] |
| AA90 | 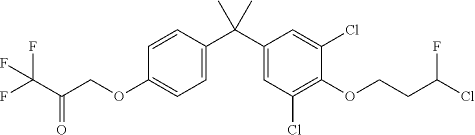 [SMILES: CC(C)(c1ccc(OCC(=O)C(F)(F)F)cc1)c1cc(Cl)c(OCCC(F)Cl)c(Cl)c1] |
| AA91 | 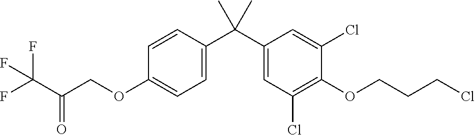 [SMILES: CC(C)(c1ccc(OCC(=O)C(F)(F)F)cc1)c1cc(Cl)c(OCCCCl)c(Cl)c1] |
| AA92 | 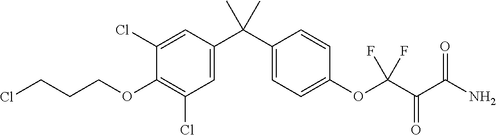 [SMILES: CC(C)(c1ccc(OC(F)(F)C(=O)C(N)=O)cc1)c1cc(Cl)c(OCCCCl)c(Cl)c1] |
| AA93 | 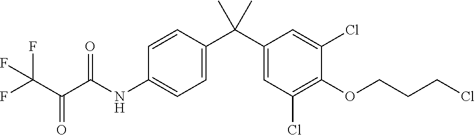 [SMILES: CC(C)(c1ccc(NC(=O)C(=O)C(F)(F)F)cc1)c1cc(Cl)c(OCCCCl)c(Cl)c1] |
| AA94 | 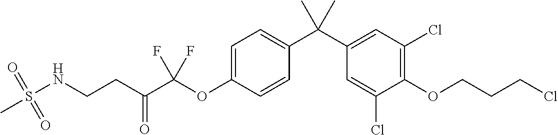 [SMILES: CC(C)(c1ccc(OC(F)(F)C(=O)CCNS(C)(=O)=O)cc1)c1cc(Cl)c(OCCCCl)c(Cl)c1] |
| AA95 | 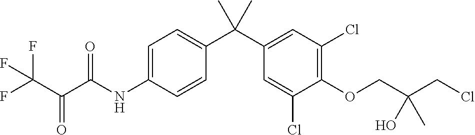 [SMILES: CC(O)(CCl)COc1c(Cl)cc(C(C)(C)c2ccc(NC(=O)C(=O)C(F)(F)F)cc2)cc1Cl] |
| AA96 | 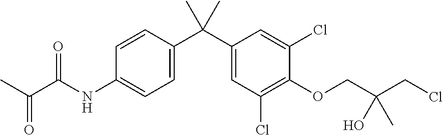 [SMILES: CC(=O)C(=O)Nc1ccc(C(C)(C)c2cc(Cl)c(OCC(C)(O)CCl)c(Cl)c2)cc1] |
| AA97 | 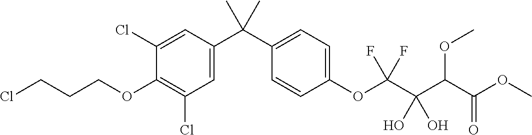 [SMILES: COC(=O)C(OC)C(O)(O)C(F)(F)Oc1ccc(C(C)(C)c2cc(Cl)c(OCCCCl)c(Cl)c2)cc1] |
| AA98 | 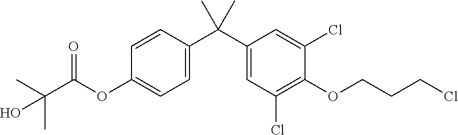 [SMILES: CC(C)(O)C(=O)Oc1ccc(C(C)(C)c2cc(Cl)c(OCCCCl)c(Cl)c2)cc1] |
| AA99 | 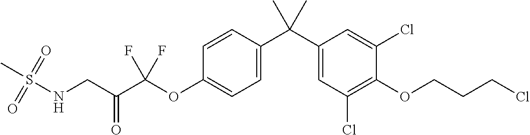 [SMILES: CC(C)(c1ccc(OC(F)(F)C(=O)CNS(C)(=O)=O)cc1)c1cc(Cl)c(OCCCCl)c(Cl)c1] |
| AA100 | 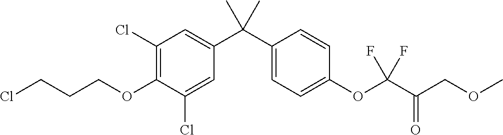 [SMILES: COCC(=O)C(F)(F)Oc1ccc(C(C)(C)c2cc(Cl)c(OCCCCl)c(Cl)c2)cc1] |
| AA101 | 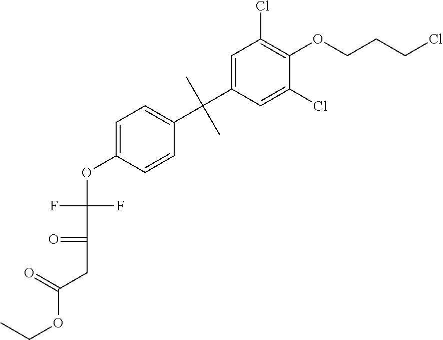 [SMILES: CCOC(=O)CC(=O)C(F)(F)Oc1ccc(C(C)(C)c2cc(Cl)c(OCCCCl)c(Cl)c2)cc1] |
| AA102 | 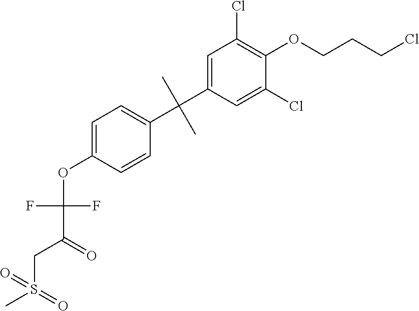 [SMILES: CC(C)(c1ccc(OC(F)(F)C(=O)CS(C)(=O)=O)cc1)c1cc(Cl)c(OCCCCl)c(Cl)c1] |
| AA103 | 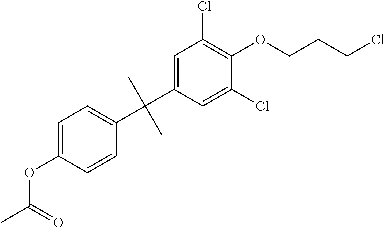 [SMILES: CC(=O)Oc1ccc(C(C)(C)c2cc(Cl)c(OCCCCl)c(Cl)c2)cc1] |
| AA104 | 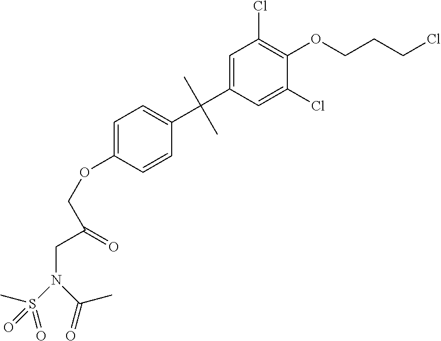 [SMILES: CC(=O)N(CC(=O)COc1ccc(C(C)(C)c2cc(Cl)c(OCCCCl)c(Cl)c2)cc1)S(C)(=O)=O] |

In one embodiment, the present invention is directed to a compound having a structure of Formula (a):

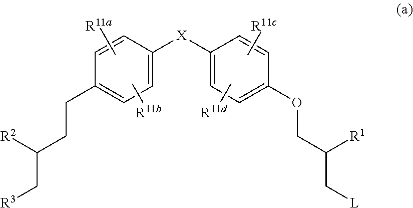

or a pharmaceutically acceptable salt, tautomer or stereoisomer thereof, wherein:
X is —S(O)ₙ— or —C(R⁸R⁹)—;
L is halogen, optionally substituted alkyl sulfonate, or optionally substituted aryl sulfonate;
R¹ is H, —OH, or —OC(═O)R¹³;
R² is —OH, or —OC(═O)R¹³;
R³ is halo, —OH, —OR⁴, —OC(═O)R¹³, —NH₂, —NHC(═O)R¹³, —N(C(═O)R¹³)₂, —NHS(O)ₙR⁵, —N(C(═O)R¹³)(S(O)ₙR⁵), —N(C₁-C₆ alkyl)(S(O)ₙR), —S(O)ₙR, —N₃, aryl, carbocyclyl, heteroaryl or heterocyclyl which are optionally substituted with one or more R⁶;
R⁴ is C₁-C₆ alkyl, C₂-C₆ alkenyl, C₂-C₆ alkynyl, aryl, carbocyclyl, heteroaryl or heterocyclyl which are optionally substituted with one or more R⁶;
R⁵ is each independently C₁-C₆ alkyl or aryl which are optionally substituted with one or more R⁶;
R⁶ is each independently selected from the group consisting of H, F, Cl, Br, I, ¹²³I, —OH, oxo, C₁-C₆ alkyl, C₂-C₆ alkenyl, C₂-C₆ alkynyl, C₁-C₆ alkoxy, C₆-C₁₂ aryl, wherein each R⁶ is optionally substituted with one or more of halogen, ¹²³I, ¹⁸F, —OH, —OS(O)₂-aryl, C₁-C₆ alkyl, C₂-C₆ alkenyl, or C₂-C₆ alkynyl;
R⁸ and R⁹ are each independently H, —OH, —NH₂, or C₁-C₆ alkyl;
R^{11a}, R^{11b}, R^{11c} and R^{11d} are each independently H, methyl, F, Cl, Br, I, ¹²³I, —OH, —NH₂, —CN, —CF₃, methyl, —COOH, or —CONH₂;
R¹³ is C₁-C₆ alkyl; and
n is 0, 1, or 2;
wherein at least one of R^{11a}, R^{11b}, R^{11c} and R^{11d} is methyl, F, Cl, Br, I, or ¹²³I.

In one embodiment of the PTCs of formula (a), X is —C(R⁸R⁹)—. In one embodiment X is —C(R⁸R⁹)—, wherein R⁸ and R⁹ are each independently H or C₁-C₃ alkyl. In another embodiment, X is —C(R⁸R⁹)—, wherein R⁸ and R⁹ are each C₁ alkyl. In some embodiments, X is —S(O)₂— or —C(CH₃)₂—. In one embodiment, R⁸ and R⁹ is each hydrogen, halogen, —OH, —NH₂, or —C₁-C₃ alkyl.

In one embodiment of the PTCs of formula (a), L is halogen, mesylate, or tosylate.

In one embodiment of the PTCs of formula (a), R¹ is —OH. In another embodiment, R¹ is —OC(═O)R¹³. In some embodiments, R¹ is —OC(═O)R¹³, wherein R¹³ is C₁-C₄ alkyl. In other embodiments, R¹ is —OC(═O)R¹³, wherein R¹³ is methyl. In one embodiment, R¹ is H.

In one embodiment of the PTCs of formula (a), R² is —OH. In another embodiment, R² is —OC(═O)R¹³. In some embodiments, R² is —OC(═O)R¹³, wherein R¹³ is C₁-C₄ alkyl. In other embodiments, R² is —OC(═O)R¹³, wherein R¹³ is methyl.

In one embodiment of the PTCs of formula (a), at least one of R¹, R², or R³ is —OH. In some embodiments, at least two of R¹, R², or R³ are each —OH. In other embodiments, R¹ and R² are each —OH.

In one embodiment of the PTCs of formula (a), at least one of R¹, R², or R³ is —OC(═O)R¹³, wherein R¹³ is C₁-C₄ alkyl. In another embodiment, at least one of R¹, R², or R³ is —OC(═O)R¹³, wherein R¹³ is methyl. In some embodiments, at least two of R¹, R², or R³ are each —OC(═O)R¹³, wherein R¹³ is C₁-C₄ alkyl. In another embodiment, at least two of R¹, R² or R³ are each —OC(═O)R¹³, wherein R¹³ is methyl. In other embodiments, R¹ and R² are each —OC(═O)R¹³, wherein R¹³ is methyl.

In one embodiment of the PTCs of formula (a), R³ is —NH₂, —NHC(═O)R¹³, —N(C(═O)R¹³)₂, —NHS(O)ₙR⁵, —N(C(═O)R¹³)(S(O)ₙR⁵), or —N(C₁-C₆ alkyl)(S(O)ₙR⁵). In one embodiment, R³ is a —NH₂. In one embodiment, R³ is a —NHC(═O)R¹³. In one embodiment, R³ is a —N(C(═O)R¹³)₂. In another embodiment, R³ is a —NHS(O)ₙR⁵. In some embodiments, R³ is a —NHS(O)₂R⁵. In other embodiments, R³ is a —NHS(O)₂R⁵, wherein R is C₁-C₃ alkyl. In one embodiment, R³ is a —NHS(O)₂R⁵, wherein R⁵ is C₁ alkyl. In one embodiment, R³ is a —N(C(═O)R¹³)(S(O)ₙR). In one embodiment, R³ is a —N(C₁-C₆ alkyl)(S(O)ₙR⁵). In one embodiment, R³ is a —NHS(O)₂CH₃.

In one embodiment of the PTCs of formula (a), R³ is —NH₂, —NHC(═O)(C1-C4 alkyl), —N[(C(═O)(C1-C4 alkyl)]₂, —NHS(O)ₙ(C1-C3 alkyl), —N[C(═O)(C1-C4 alkyl)][(S(O)ₙ(C1-C3 alkyl)], or —N[C1-C6 alkyl][S(O)ₙ(C1-C3 alkyl)]. In some embodiments, R³ is —NH(C(═O)CH₃) or —N(C(═O)CH₃)₂. In other embodiments, R³ is —NHS(O)₂CH₃. In other embodiments, R³ is —N(C(═O)CH₃) (S(O)₂CH₃).

In one embodiment of the PTCs of formula (a), R³ is a —S(O)ₙR⁵. In one embodiment, R³ is a —S(O)₂R⁵. In another embodiment, R³ is a —S(O)₂(C1-C3 alkyl). In other embodiments, R³ is a —S(O)₂CH₃. In other embodiments, R³ is a —S(O)₂CH₂CH₃.

In one embodiment of the PTCs of formula (a), R³ is an optionally substituted 5 or 6 membered heteroaryl or an optionally substituted 3 to 7 membered heterocylyl, wherein said heteroaryl or said heterocyclyl respectively comprise at least one N atom in the ring. In one embodiment, R³ is selected from a group consisting of pyrrole, furan, thiophene, pyrazole, pyridine, pyridazine, pyrimidine, imidazole, thiazole, isoxazole, oxadiazole, thiadiazole, oxazole, triazole, isothiazole, oxazine, triazine, azepine, pyrrolidine, pyrroline, imidazoline, imidazolidine, pyrazoline, pyrazolidine, piperidine, dioxane, morpholine, dithiane, thiomorpholine, piperazine, and tetrazine. In a certain embodiment, R³ is

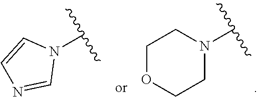 [SMILES: *n1ccnc1 ; *N1CCOCC1]

In one embodiment of the PTCs of formula (a), R³ is —OR⁴. In one embodiment, R³ is —OR⁴, wherein R⁴ is C₁-C₆ alkyl. In another embodiment, R³ is —OR⁴, wherein R⁴ is C₁-C₃ alkyl. In one embodiment, R³ is —OR⁴, wherein R⁴ is methyl, ethyl, n-propyl, or i-propyl. In one embodiment, R³ is —OR⁴, wherein R⁴ is methyl. In another embodiment, R³ is —OR⁴, wherein R⁴ is i-propyl.

In one embodiment of the PTCs of formula (a), R³ is a halogen. In other embodiments, R³ is F, Cl, Br, or I. In one embodiment, R³ is F.

In one embodiment of the PTCs of formula (a), at least one of R^{11a}, R^{11b}, R^{11c} and R^{11d} is Cl. In another embodiment, at least one of R^{11a}, R^{11b}, R^{11c} and R^{11d} is Br. In some embodiments, at least one of R¹¹, R^{11b}, R^{11c} and R^{11d} is methyl.

In one embodiment of the PTCs of formula (a), at least two of R^{11a}, R^{11b}, R^{11c} and R^{11d} are methyl, F, Cl, Br, I, or ¹²³I. In another embodiment, exactly two of R^{11a}, R^{11b}, R^{11c} and R^{11d} are methyl, F, Cl, Br, I, or ¹²³I.

In one embodiment of the PTCs of formula (a), R^{11a} and R^{11b} are each H and R^{11c} and R^{11d} are each independently methyl, F, Cl, Br, I, or ¹²³I. In one embodiment, R^{11a} and R^{11b} are each H, and R^{11c} and R^{11d} are each Cl. In one embodiment, R^{11a} and R^{11b} are each H, and R^{11c} and R^{11d} are each Br. In one embodiment, R^{11a} and R^{11b} are each H, and R^{11c} and R^{11d} are each methyl.

In one embodiment of the PTCs of formula (a), R^{11a} and R^{11c} are each H, and R^{11b} and R^{11d} are each independently methyl, F, Cl, Br, I, or ¹²³I. In one embodiment, R^{11a} and R^{11c} are each H, and R^{11b} and R^{11d} are each Cl. In one embodiment, R^{11a} and R^{11c} are each H, and R^{11b} and R^{11d} are each Br. In one embodiment, R^{11a} and R^{11c} are each H, and R^{11b} and R^{11d} are each methyl.

In one embodiment of the PTCs of formula (a), R^{11a}, R^{11b}, R^{11c} and R^{11d} is, each independently, H, halogen, —OH, —NH₂, —CN, —CF₃, methyl, —COOH, or —CONH₂.

In one embodiment of the PTCs of formula (a), R¹³ is C₁-C₃ alkyl. In other embodiments, R¹³ is methyl, ethyl, or propyl. In one embodiment, R¹³ is a methyl.

In one embodiment of the PTCs of formula (a), n is 0. In another embodiment n is 1. In some embodiments, n is 2.

In one embodiment of the PTCs of formula (a), the PTCs comprises one or more of F, Cl, Br, I or ¹²³I substitutions for R³. In one embodiment, the PTCs comprises one or more of I or ¹²³I substitutions for R³.

In one embodiment of the PTCs of formula (a), the PTCs comprises at least one R⁶ substituent on R³, wherein at least one R⁶ is further substituted with at least one of F, Cl, Br, I or ¹²³I. In another embodiment, R⁶ substituent on R³ is further substituted with at least one of I or ¹²³I.

In some more specific embodiments of the PTCs of Formula (a), the PTC has one of the following structures from Table D, or a pharmaceutically acceptable salt, tautomer, or stereoisomer thereof. In one embodiment, the PTCs of Formula (a) is selected from Compounds 1, 1a, 1A, 1aA, 5, 5a, 5A, 5aA, 7, 7a, 7A, 7aA, 8, 8a, 8A, 8aA, 9, 9a, 9A, 9aA, 11, 11a, 11A, 11aA, 12, 12a, 13, 13a, 13A, 13aA, 14, 14a, 14A, 14aA, 22, or 22a, or a pharmaceutically acceptable salt, tautomer, or stereoisomer thereof.

In one embodiment of the PTCs of formula (a), the PTC is connected to the linker LI through R¹, R², R³, R⁴, R⁵, R^{11a}, R^{11b}, R^{11c}, R^{11d} and R¹³. In one embodiment of the PTCs of formula (a), the PTC forms a covalent bond with the linker LI through standard organic chemistry protocols, such as substitution reactions and amino acid coupling reactions.

In one embodiment of the PTCs of formula (a), a hydrogen (e.g., C—H, N—H, O—H, S—H), halogen, sulfonates (e.g., tosylate, mesylate), or any chemical group as defined in the formula (Ia) is used to form the covalent bond between PTC and the linker LI. Thus, it can be understood that PTCs as disclosed herein as a neutral molecule is intended to be covalently bonded to the linker LI to form the protac molecule of formula (Q) by at least displacing one atom or one chemical group (e.g., H, halogen, OH, NH₂, OTs, OMs, etc) from the PTCs of formula (a) to form the covalent bond with LI.

In one embodiment, PTC in formula Q is a compound of formula (a), minus any functional group that was involved in making the PTC-LI bond.

The compounds disclosed in WO 2017/177307 can be useful PTCs for the present invention. The disclosure of WO 2017/177307 is incorporated by reference in its entirety for all purposes.

TABLE D
PTCs

| Compd | |
|---|---|
| ID | Structure |
| 1 | 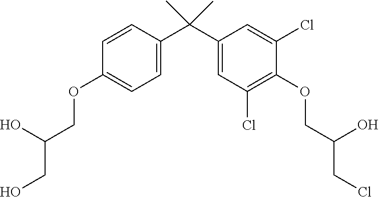 [SMILES: CC(C)(c1ccc(OCC(O)CO)cc1)c1cc(Cl)c(OCC(O)CCl)c(Cl)c1] |
| 1A | 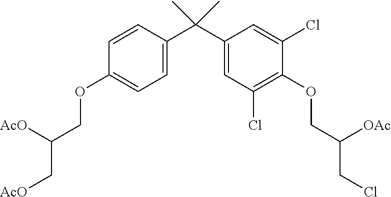 [SMILES: CC(=O)OCC(COc1ccc(C(C)(C)c2cc(Cl)c(OCC(CCl)OC(C)=O)c(Cl)c2)cc1)OC(C)=O] |
| 1a | 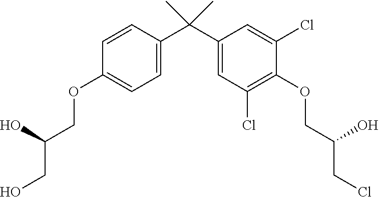 [SMILES: CC(C)(c1ccc(OC[C@H](O)CO)cc1)c1cc(Cl)c(OC[C@H](O)CCl)c(Cl)c1] |
| 1aA | 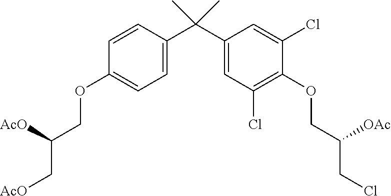 [SMILES: CC(=O)OC[C@H](COc1ccc(C(C)(C)c2cc(Cl)c(OC[C@@H](CCl)OC(C)=O)c(Cl)c2)cc1)OC(C)=O] |
| 5 | 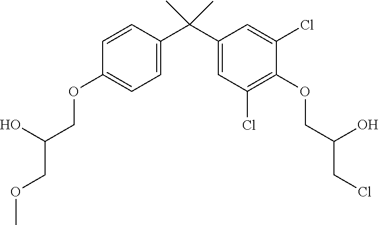 [SMILES: COCC(O)COc1ccc(C(C)(C)c2cc(Cl)c(OCC(O)CCl)c(Cl)c2)cc1] |
| 5A | 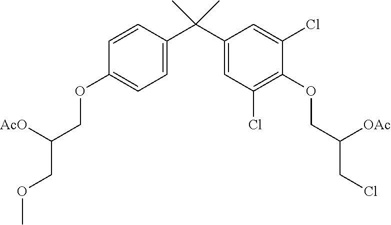 [SMILES: COCC(COc1ccc(C(C)(C)c2cc(Cl)c(OCC(CCl)OC(C)=O)c(Cl)c2)cc1)OC(C)=O] |
| 5a | 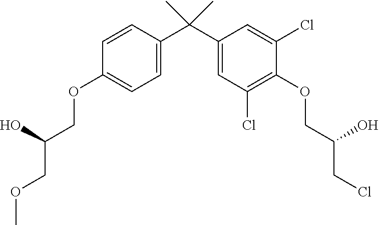 [SMILES: COC[C@@H](O)COc1ccc(C(C)(C)c2cc(Cl)c(OC[C@H](O)CCl)c(Cl)c2)cc1] |
| 5aA | 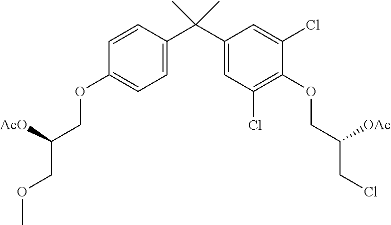 [SMILES: COC[C@H](COc1ccc(C(C)(C)c2cc(Cl)c(OC[C@@H](CCl)OC(C)=O)c(Cl)c2)cc1)OC(C)=O] |
| 7 | 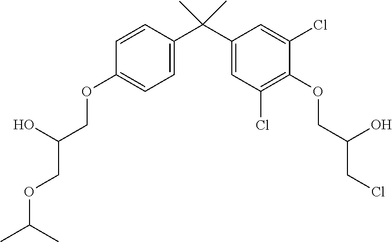 [SMILES: CC(C)OCC(O)COc1ccc(C(C)(C)c2cc(Cl)c(OCC(O)CCl)c(Cl)c2)cc1] |
| 7A | 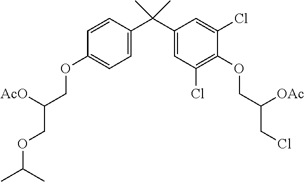 [SMILES: CC(=O)OC(CCl)COc1c(Cl)cc(C(C)(C)c2ccc(OCC(COC(C)C)OC(C)=O)cc2)cc1Cl] |
| 7a | 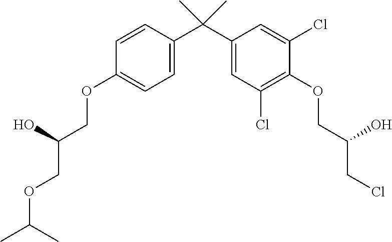 [SMILES: CC(C)OC[C@@H](O)COc1ccc(C(C)(C)c2cc(Cl)c(OC[C@H](O)CCl)c(Cl)c2)cc1] |
| 7aA | 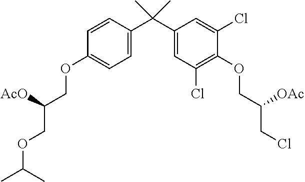 [SMILES: CC(=O)O[C@H](CCl)COc1c(Cl)cc(C(C)(C)c2ccc(OC[C@@H](COC(C)C)OC(C)=O)cc2)cc1Cl] |
| 8 | 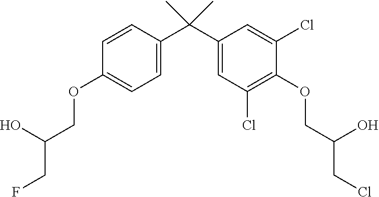 [SMILES: CC(C)(c1ccc(OCC(O)CF)cc1)c1cc(Cl)c(OCC(O)CCl)c(Cl)c1] |
| 8A | 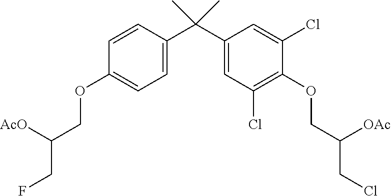 [SMILES: CC(=O)OC(CF)COc1ccc(C(C)(C)c2cc(Cl)c(OCC(CCl)OC(C)=O)c(Cl)c2)cc1] |
| 8a | 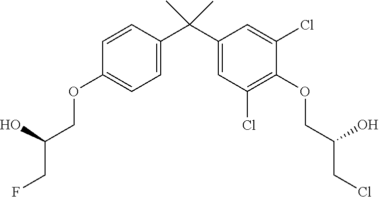 [SMILES: CC(C)(c1ccc(OC[C@H](O)CF)cc1)c1cc(Cl)c(OC[C@H](O)CCl)c(Cl)c1] |
| 8aA | 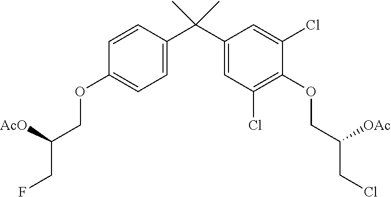 [SMILES: CC(=O)O[C@H](CF)COc1ccc(C(C)(C)c2cc(Cl)c(OC[C@@H](CCl)OC(C)=O)c(Cl)c2)cc1] |
| 9 | 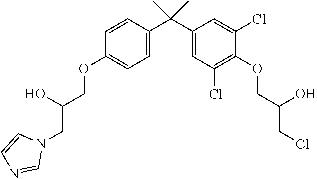 [SMILES: CC(C)(c1ccc(OCC(O)Cn2ccnc2)cc1)c1cc(Cl)c(OCC(O)CCl)c(Cl)c1] |
| 9A | 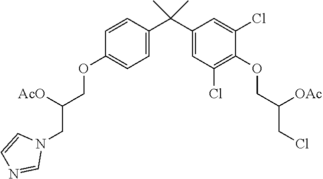 [SMILES: CC(=O)OC(CCl)COc1c(Cl)cc(C(C)(C)c2ccc(OCC(Cn3ccnc3)OC(C)=O)cc2)cc1Cl] |
| 9a | 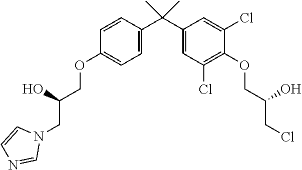 [SMILES: CC(C)(c1ccc(OC[C@H](O)Cn2ccnc2)cc1)c1cc(Cl)c(OC[C@H](O)CCl)c(Cl)c1] |
| 9aA | 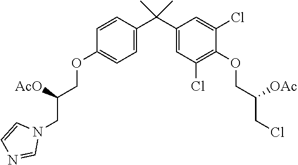 [SMILES: CC(=O)O[C@H](CCl)COc1c(Cl)cc(C(C)(C)c2ccc(OC[C@@H](Cn3ccnc3)OC(C)=O)cc2)cc1Cl] |
| 11 | 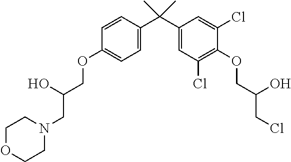 [SMILES: CC(C)(c1ccc(OCC(O)CN2CCOCC2)cc1)c1cc(Cl)c(OCC(O)CCl)c(Cl)c1] |
| 11A | 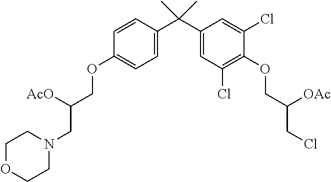 [SMILES: CC(=O)OC(CCl)COc1c(Cl)cc(C(C)(C)c2ccc(OCC(CN3CCOCC3)OC(C)=O)cc2)cc1Cl] |
| 11a | 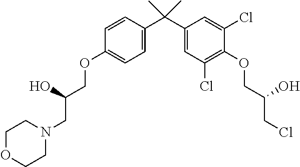 [SMILES: CC(C)(c1ccc(OC[C@H](O)CN2CCOCC2)cc1)c1cc(Cl)c(OC[C@H](O)CCl)c(Cl)c1] |
| 11aA | 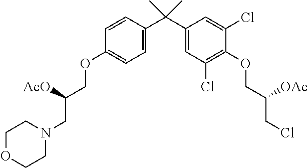 [SMILES: CC(=O)O[C@H](CCl)COc1c(Cl)cc(C(C)(C)c2ccc(OC[C@@H](CN3CCOCC3)OC(C)=O)cc2)cc1Cl] |
| 12 | 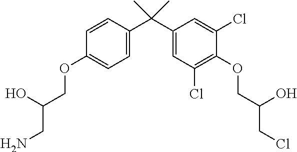 [SMILES: CC(C)(c1ccc(OCC(O)CN)cc1)c1cc(Cl)c(OCC(O)CCl)c(Cl)c1] |
| 12a | 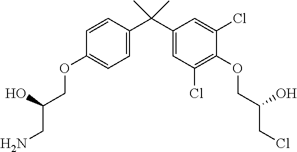 [SMILES: CC(C)(c1ccc(OC[C@H](O)CN)cc1)c1cc(Cl)c(OC[C@H](O)CCl)c(Cl)c1] |
| 13 | 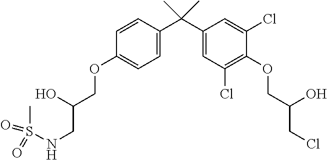 [SMILES: CC(C)(c1ccc(OCC(O)CNS(C)(=O)=O)cc1)c1cc(Cl)c(OCC(O)CCl)c(Cl)c1] |
| 13A | 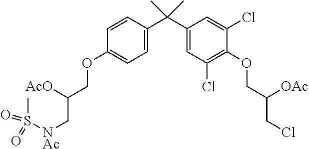 |
| 13a | 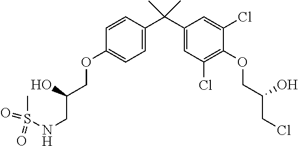 [SMILES: CC(C)(c1ccc(OC[C@H](O)CNS(C)(=O)=O)cc1)c1cc(Cl)c(OC[C@H](O)CCl)c(Cl)c1] |
| 13aA | 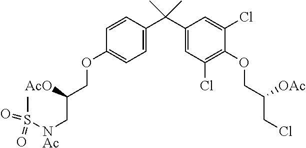 |
| 14 | 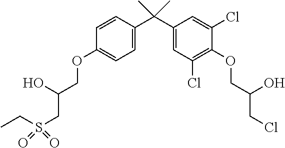 [SMILES: CCS(=O)(=O)CC(O)COc1ccc(C(C)(C)c2cc(Cl)c(OCC(O)CCl)c(Cl)c2)cc1] |
| 14A | 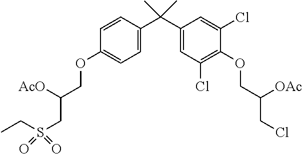 [SMILES: CCS(=O)(=O)CC(COc1ccc(C(C)(C)c2cc(Cl)c(OCC(CCl)OC(C)=O)c(Cl)c2)cc1)OC(C)=O] |
| 14a | 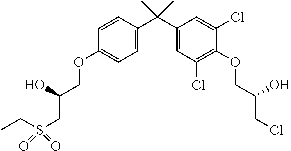 [SMILES: CCS(=O)(=O)C[C@@H](O)COc1ccc(C(C)(C)c2cc(Cl)c(OC[C@H](O)CCl)c(Cl)c2)cc1] |
| 14aA | 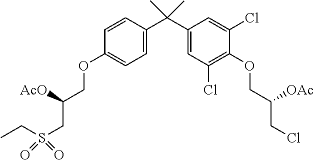 [SMILES: CCS(=O)(=O)C[C@H](COc1ccc(C(C)(C)c2cc(Cl)c(OC[C@@H](CCl)OC(C)=O)c(Cl)c2)cc1)OC(C)=O] |
| 22 | 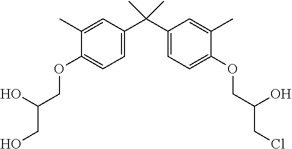 [SMILES: Cc1cc(C(C)(C)c2ccc(OCC(O)CCl)c(C)c2)ccc1OCC(O)CO] |
| 22a | 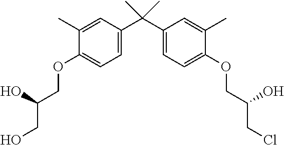 [SMILES: Cc1cc(C(C)(C)c2ccc(OC[C@H](O)CCl)c(C)c2)ccc1OC[C@H](O)CO] |

In one embodiment, any of the PTCs disclosed herein can further comprise a chemical group useful in covalently attaching the PTC to the LI. In one embodiment, any of the PTCs disclosed herein can be derivatized with a chemical group useful in covalently attaching the PTC to the LI. In one embodiment, any of the PTCs disclosed herein can be derivatized with a chemical group useful in covalently attaching the PTC to the LI. In one embodiment, the derivatization may include small linking group that can be covalently attach to LI (e.g., —NH—; —OC(O)NH—; —OC(O)—, etc).

In one embodiment, the PTCs as disclosed herein is an androgen receptor modulator. In one embodiment, the PTCs as disclosed herein binds to androgen receptor. In another embodiment, the PTCs as disclosed herein binds to androgen receptor N-terminal domain.

In one embodiment, the present disclosure provides a pharmaceutical composition comprising a pharmaceutically acceptable carrier or a pharmaceutically acceptable excipient and a compound wherein the PTC is selected from any one of formula (I)-(V) or compounds of Tables A and B, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound wherein the PTC is selected from Compounds A1-A186 or B1-B11, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound wherein the PTC is selected from any one of formula (i)-(iv) or compounds of Table C, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound wherein the PTC is selected from Compounds AA1-AA98, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound of wherein the PTC is selected from formula (a) or compounds of Table D, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound wherein the PTC is selected from Compounds 1, 1a, 1A, 1aA, 5, 5a, 5A, 5aA, 7, 7a, 7A, 7aA, 8, 8a, 8A, 8aA, 9, 9a, 9A, 9aA, 11, 11a, 11A, 11aA, 12, 12a, 13, 13a, 13A, 13aA, 14, 14a, 14A, 14aA, 22, or 22a, or a pharmaceutically acceptable salt, tautomer, stereoisomer or prodrug thereof.

In one embodiment, the present disclosure provides a pharmaceutical composition comprising a pharmaceutically acceptable carrier or a pharmaceutically acceptable excipient and a PTC wherein the PTC is selected from a PTC of any one of formula (I), (IA), (IB), (IC), (II), (IIA), (IIIA), (IIB), (III), (IV), (IVA), (V), (VA), (VI), (A), (A-I), (B)-(D), (E), (E-I)-(E-VII), (F), (G), (G-I), (G-II), (H), and (H-I) (“formula (I)-(VI) and (A)-(H-I)”) or PTCs of Tables A and B, or a pharmaceutically acceptable salt, tautomer, stereoisomer or prodrug thereof.

Protein Target Compounds (PTCs) with a Linker (LI) Containing a Functional Group

In one embodiment, the present disclosure includes PTC-LI_{A} compound where the LI_{A} is a linker (LI) with a functional group (FG) useful in reacting with a ligase modulator compound to form a compound of formula (Q).

In one embodiment of PTC-LI_{A}, PTC can be any PTC disclosed herein, for example compounds of formula formula (I)-(VI), (A)-(H-I), (i)-(iv), and (a) or any compounds in Tables A-D.

In one embodiment of PTC-LI_{A}, LI_{A} is any linker (LI) disclosed herein which contains functional group (FG) selected from carboxylic acid, aldehyde, hydroxyl, alkoxyl, aryloxy-, halogen, amine, amide, azide, alkynyl, or sulfonates (e.g., tosylate, mesylate, triflate, etc.).

In one embodiment of PTC-LI_{A}, LI_{A} has the structure -LI-FG. In one embodiment, PTC-LI_{A} has the structure selected from PTC-LI-COOH, PTC-LI-COH, PTC-LI-OH, PTC-LI-O-alkyl, PTC-LI-O-aryl, PTC-LI-I (iodine), PTC-LI-Br, PTC-LI-Cl, PTC-LI-F, PTC-LI-NH₂, PTC-LI-NH(alkyl), PTC-LI-NH(aryl), PTC-LI-NHCO(alkyl), PTC-LI-N(alkyl)CO(alkyl), PTC-LI-CONH₂, PTC-LI-CONH(alkyl), PTC-LI-CONH(aryl), PTC-LI-N₃, PTC-LI-C≡CH, PTC-LI-C≡C(alkyl), PTC-LI-OSO₂(alkyl), PTC-LI-OSO₂(haloalkyl), or PTC-LI-OSO₂(aryl), wherein PTC and LI are as disclosed herein.

In some embodiments, PTC-LI_{A} is a compound of formula (Y-IV), (Y-IVA), (Y-V), (Y-VA), (Y-VI), (Y-VIA), (Y-VII), (Y-VIII), (Y-IX), or (Y-X):

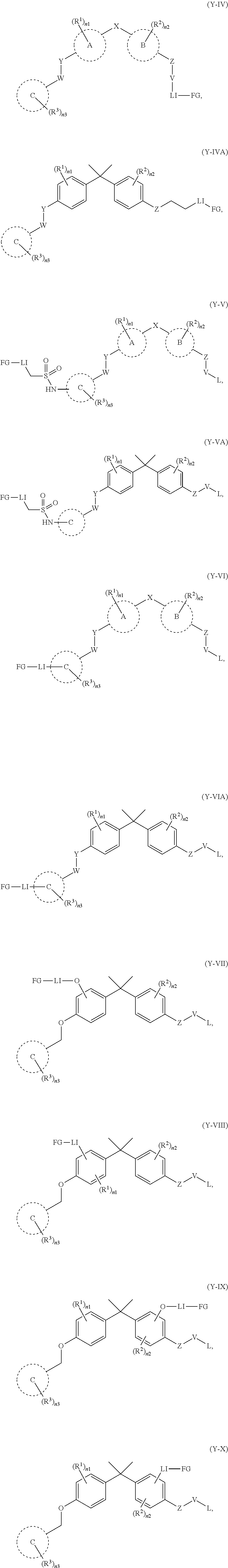

or a pharmaceutically acceptable salt thereof, wherein A, B, C, R¹, R², R³, Z, V, L, Y, W, LI, FG, n1, n2, and n3 are as defined herein.

In some embodiments, PTC-LI_{A} is selected from

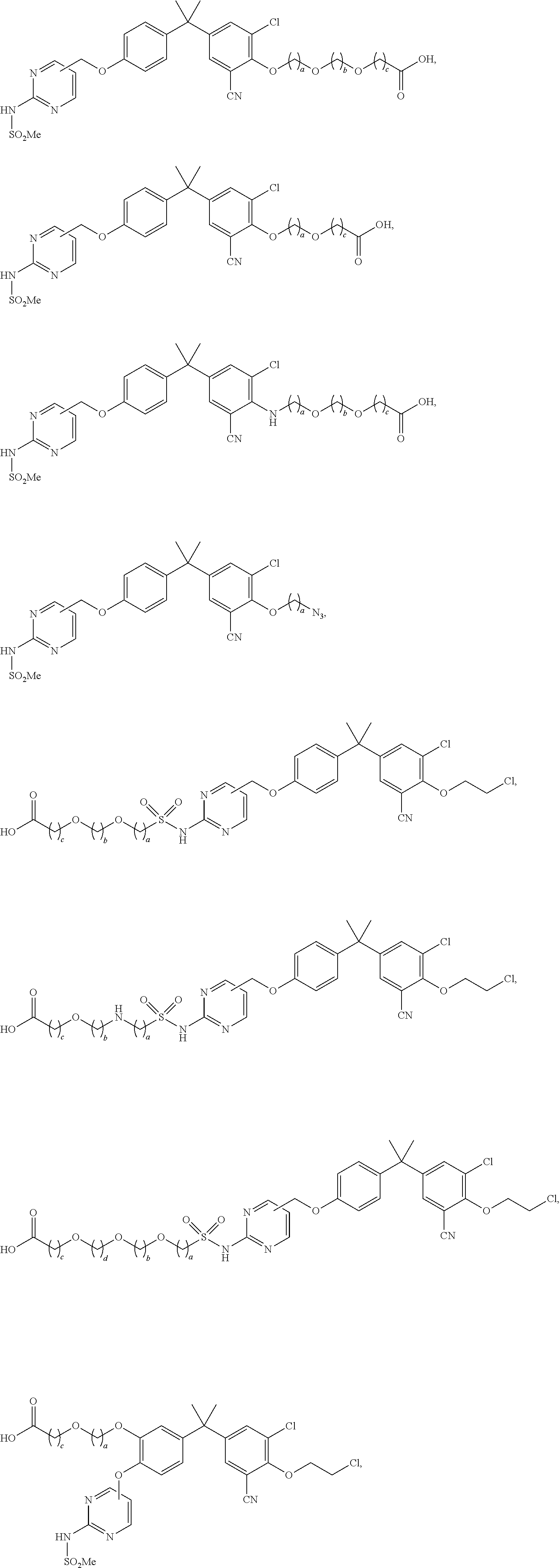

or a pharmaceutically acceptable salt thereof, wherein a, b, c, and d are each independently an integer between 1 to 10. In one embodiment, a is 5, b is 3, and c is 1. In one embodiment, a is 2, b is 5, and c is 1. In one embodiment, a is 2, b is 5, c is 1, and d is 3. In one embodiment, a is 5 and c is 1. In one embodiment, a is 5. In one embodiment, a is 3.

Ligase Modulators (PLMs)

In one embodiment, any of the PLMs disclosed herein can be the PLM as covalently attached to the LI. In some embodiments, any of the PLMs disclosed herein can be the ligase modulator moiety before covalently attaching it to the LI. In a non limited example, the PLM can comprise a chemical group (e.g., alcohol, amine, azides, —C≡CH, etc) which can be reacted with another chemical group on or attached to the LI in order to form a covalent bond, e.g., amine bond, ether bond, amide bond, ester bond, triazole (Click chemistry). In one embodiment, a chemical group already present in the PLM as described here can be used to covalently attach the PLM to the LI. The chemistry used to covalently attach the LI to the PLM can be readily understood by one skilled in the art.

In one embodiment, any of the PLMs disclosed herein can further comprise a chemical group useful in covalently attaching the PLM to the LI. In one embodiment, any of the PLMs disclosed herein can be derivatized with a chemical group useful in covalently attaching the PLM to the LI. In one embodiment, any of the PLMs disclosed herein can be derivatized with a chemical group useful in covalently attaching the PLM to the LI. In one embodiment, the derivatization may include small linking group that can be covalently attach to LI (e.g., —NH—; —OC(O)NH—; —OC(O)—, etc).

In one embodiment, the PLMs of the present disclosure are E3 ligases or comprises an E3 ligase recognition domain.

In one embodiment, the PLM is thalidomide, pomalidomide, or lenalidomide, or derivatives thereof. See E. S. Fischer, et al. Nature 2014, 512, 49-53.

In one embodiment, the PLM is a von Hippel Lindau (VHL) ligand, a celeblon, a mouse double minute 2 homolog (MDM2) or an inhibitor of apoptosis (IAP).

In one embodiment, the PLM is a von Hippel Lindau (VHL) ligand which binds to the VHL E3 ubiquitin ligase, including but not limited to those disclosed in C. M. Crews, et al. Oncogene 2008, 27, 7201; C. M. Crews, et al. Angew. Chem. Int. Ed. 2012, 51, 11463; WO 2013/106646, WO 2016/118666, WO 2016/149668, WO 2017/011590, and/or WO 2019/023553, each disclosure are hereby incorporated by reference in their entireties for all purposes.

In one embodiment, the PLM is a moiety specific for an E3 ubiquitin ligase. In one embodiment, the PLM is an E3 ligase substrate receptor cereblon (CRBN). Examples of celeblon ligands are disclosed in U.S. Pat. No. 9,750,816 and Wustrow, D.; Zhou, H.-J.; Rolfe, M., Annu. Rep. Med. Chem. 2013, 48, 205-225, the disclosures of which are hereby incorporated by reference in their entireties for all purposes.

In one embodiment, the PLM is a mouse double minute 2 homolog (MDM2). In cancer patients, about 50% were found with p53 mutation (M. Hollstein, et al. Science (1991), 233, 49-53), while patients with wild type p53 were often found p53 down regulation by MDM2 through the protein-protein interaction of p53 and MDM2 (P. Chene, et al. Nat. Rev. Cancer (2003), 3, 102-109). Under normal cell condition without oncogenic stress signal, MDM2 keeps p53 at low concentration. In response to DNA damage or cellular stress, p53 level increases, and that also causes increase in MDM2 due to the feedback loop from p53/MDM2 auto regulatory system. In other words, p53 regulates MDM2 at the transcription level, and MDM2 regulates p53 at its activity level (A. J. Levine, et al. Genes Dev. (1993) 7, 1126-1132). Several mechanisms can explain p53 down regulation by MDM2. First, MDM2 binds to N-terminal domain of p53 and blocks expression of p53-responsive genes (J. Momand, et al. Cell (1992), 69, 1237-1245). Second, MDM2 shuttles p53 from nucleus to cytoplasm to facilitate proteolytic degradation (J. Roth, et al. EMBO J. (1998), 17, 554-564). Lastly, MDM2 carries intrinsic E3 ligase activity of conjugating ubiquitin to p53 for degradation through ubiquitin-dependent 26s proteasome system (UPS) (Y. Haupt, et al. Nature (1997) 387, 296-299). Therefore, disrupting p53/MDM2 auto regulation can restore p53 activity and could bring a new approach in the treatment of cancer. See WO 2017/011371 and Wustrow, D. et al., Annu. Rep. Med. Chem. 2013, 48, 205-225, which are hereby incorporated by reference in their entirety.

In one embodiment, the PLM is a human double minute 2 homolog (HDM2). See Wustrow, D. et al., Annu. Rep. Med. Chem. 2013, 48, 205-225, which are hereby incorporated by reference in their entirety.

In one embodiment, the PLM is an inhibitor of apoptosis (IAP). IAPs are a protein family involved in suppressing apoptosis, i.e. cell death. The human IAP family includes 8 members, and numerous other organisms contain IAP homologs. IAPs contain an E3 ligase specific domain and baculoviral IAP repeat (BIR) domains that recognize substrates, and promote their ubiquitination. IAPs promote ubiquitination and can directly bind and inhibit caspases. Caspases are proteases (e.g. caspase-3, caspase-7 and caspace-9) that implement apoptosis. As such, through the binding of caspases, IAPs inhibit cell death.

In one embodiment, the PLM has the structure of formula (E3A):

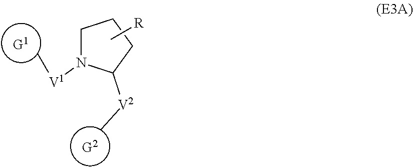

wherein:

V¹, V² are each independently a bond, O, NR^{a}, CR^{8a}R^{b}, C═O, C═S, SO, SO₂;

R^{a} and R^{b} are each independently H, linear or branched C₁₋₆ alkyl, optionally substituted by 1 or more halo, or C₁₋₆ alkoxyl optionally substituted with 0 to 3 R;

R is 0, 1, 2, or 3, groups, each independently selected from H, halo, —OH, C₁₋₃ alkyl, or C═O;

G¹ is an optionally substituted -T-N(R^{1a}R^{1b}), -T-aryl, an optionally substituted -T-heteroaryl, an optionally substituted -T-heterocycle, an optionally substituted —NR¹-T-aryl, an optionally substituted —NR¹-T-heteroaryl or an optionally substituted —NR¹-T-heterocycle, where T is covalently bonded to V¹;

each R¹, R^{1a} and R^{1b} is independently H, a C₁-C₆ alkyl group (linear, branched, optionally substituted by 1 or more halo, —OH), R^{a}C═O, R^{a}C═S, R^{a}SO, R^{a}SO₂, N(R^{a}R^{b})C═O, N(R^{a}R^{b})C═S, N(R^{a}R^{b})SO, N(R^{a}R^{b})SO₂;

V² is an optionally substituted —NR¹-T-aryl, an optionally substituted —NR¹-T-heteroaryl group or an optionally substituted —NR¹-T-heterocycle, wherein —NR¹ is covalently bonded to X²; R¹ is H or CH₃, preferably H; and

T is an optionally substituted —(CH₂)ₙ— group, wherein each one of the methylene groups may be optionally substituted with one or two substituents, preferably selected from halogen, a C₁-C₆ alkyl group (linear, branched, optionally substituted by 1 or more halogen, —OH) or the sidechain of an amino acid as otherwise described herein, preferably methyl, which may be optionally substituted; and n is 0 to 6; wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, any one of the hydrogen atoms in the PLM can be replaced to form a covalent bond to the LI.

In one embodiment, the PLM has the structure of formula (E3B):

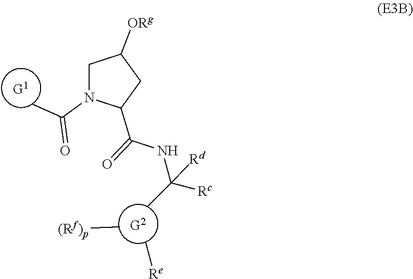

wherein, G¹ is optionally substituted aryl, optionally substituted heteroaryl, or —CR⁹R¹⁰R¹¹;

each R⁹ and R¹⁰ is independently hydrogen, optionally substituted alkyl, optionally substituted cycloalkyl, optionally substituted hydroxyalkyl, optionally substituted heteroaryl, or haloalkyl; or R⁹ and R¹⁰ and the carbon atom to which they are attached form an optionally substituted cycloalkyl;

R¹¹ is optionally substituted heterocyclic, optionally substituted alkoxy, optionally substituted heteroaryl, optionally substituted aryl, or —NR¹²R¹³,

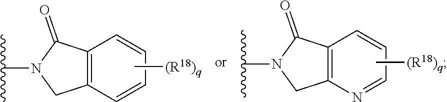

R¹² is H or optionally substituted alkyl;

R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;

R^{c} and R^{d} is each independently H, haloalkyl, or optionally substituted alkyl;

G² is a phenyl or a 5-10 membered heteroaryl,

R^{e} is H, halogen, CN, OH, NO₂, NR^{c}R^{d}, OR^{c}R, CONR^{c}R^{d}, NR^{c}COR^{d}, SO₂NR^{c}R^{d}, NR^{c}SO₂R^{d}, optionally substituted alkyl, optionally substituted haloalkyl, optionally substituted haloalkoxy; optionally substituted aryl; optionally substituted heteroaryl; optionally substituted cycloalkyl; optionally substituted cycloheteroalkyl;

each R^{f} is independently halo, optionally substituted alkyl, haloalkyl, hydroxy, optionally substituted alkoxy, or haloalkoxy;

R^{g} is H, C₁₋₆alkyl, —C(O)R¹⁹; —C(O)OR¹⁹; or —C(O)NR¹⁹R¹⁹;

p is 0, 1, 2, 3, or 4;

each R¹⁸ is independently halo, optionally substituted alkoxy, cyano, optionally substituted alkyl, haloalkyl, haloalkoxy or a linker;

each R¹⁹ is independently H, optionally substituted alkyl, or optionally substituted aryl;

q is 0, 1, 2, 3, or 4; and

wherein any one of the hydrogen atoms in the PLM can be replaced to form a covalent bond to the LI.

In one embodiment, the PLM has the structure of formula (E3C):

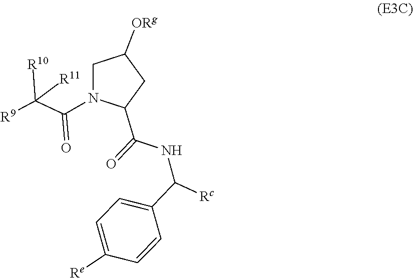

wherein, R⁹ is H;

R¹⁰ is isopropyl, tert-butyl, sec-butyl, cyclopentyl, or cyclohexyl;

R¹¹ is —NR¹²R¹³;

R¹² is H;

R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;

R^{c} is H, haloalkyl, methyl, ethyl, isopropyl, cyclopropyl, or C₁-C₆ alkyl (linear, branched, optionally substituted), each optionally substituted with 1 or more halo, hydroxyl, nitro, CN, C₁-C₆ alkyl (linear, branched, optionally substituted), or C₁-C₆ alkoxyl (linear, branched, optionally substituted);

R^{e} is

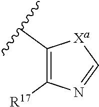

wherein R¹⁷ is H, halo, optionally substituted C₃₋₆cycloalkyl, optionally substituted C₁₋₆alkyl, optionally substituted C₁₋₆alkenyl, or C₁₋₆haloalkyl; and X^{a} is S or O; and wherein any one of the hydrogen atoms in the PLM can be replaced to form a covalent bond to the LI.

In one embodiment, the PLM has the structure of formula (E3D):

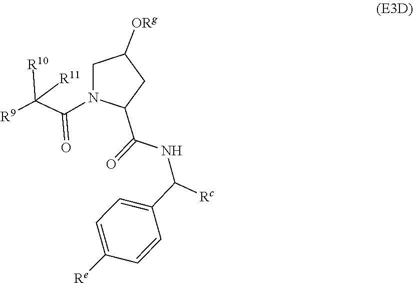

wherein, R⁹ is H;

R¹⁰ is C₁₋₆ alkyl;

R¹¹ is —NR¹²R¹³;

R¹² is H;

R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;

R^{c} is H, haloalkyl, methyl, ethyl, isopropyl, cyclopropyl, or C₁-C₆ alkyl (linear, branched, optionally substituted), each optionally substituted with 1 or more halo, hydroxyl, nitro, CN, C₁-C₆ alkyl (linear, branched, optionally substituted), or C₁-C₆ alkoxyl (linear, branched, optionally substituted); and

R^{e} is

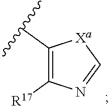

wherein R¹⁷ is H, halo, optionally substituted C₃₋₆cycloalkyl, optionally substituted C₁₋₆alkyl, optionally substituted C₁₋₆alkenyl, or C₁₋₆haloalkyl; and X^{a} is S or O;

R⁹ is H, C₁₋₆ alkyl, —C(O)R¹⁹; —C(O)OR¹⁹; or —C(O)NR¹⁹R¹⁹;

R¹⁹ is independently H, optionally substituted alkyl, or optionally substituted aryl; and

wherein any one of the hydrogen atoms in the PLM can be replaced to form a covalent bond to the LI.

In one embodiment of the PLMs of formula (E3A)-(E3D), the connectivity to the linker LI is at R¹³.

In some embodiments of the compound of formula (Q), the PLM is represented by formula (W-II):

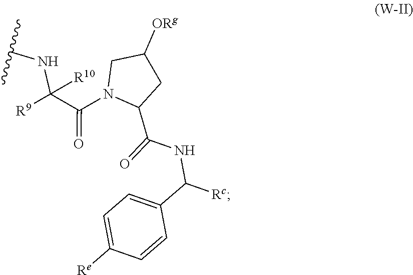

wherein the PLM is covalently bound to the LI via

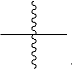

In one embodiment, the PLM is selected from

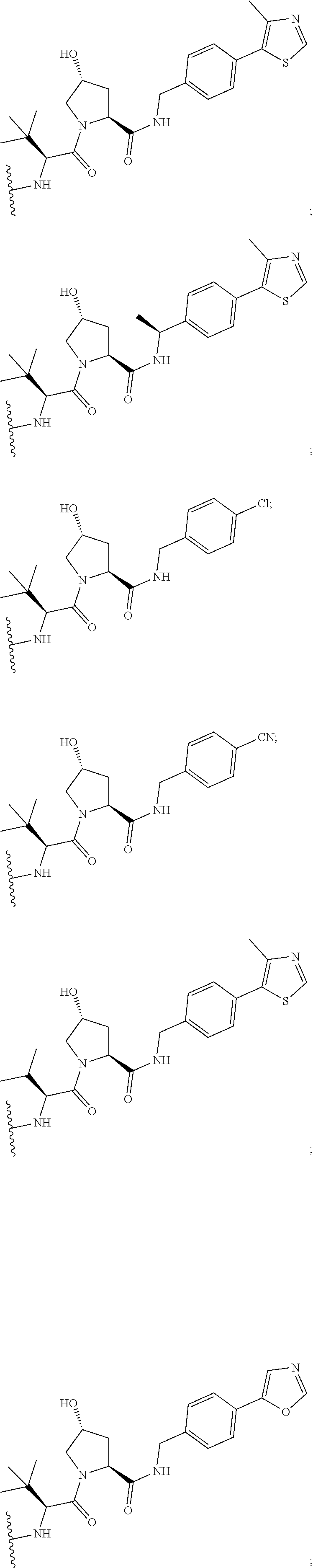 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(Cl)cc1)C(C)(C)C ; *N[C@H](C(=O)N1C[C@H](O)C[C@H]1C(=O)NCc1ccc(C#N)cc1)C(C)(C)C ; *N[C@H](C(=O)N1C[C@H](O)C[C@H]1C(=O)NCc1ccc(-c2scnc2C)cc1)C(C)C ; *N[C@H](C(=O)N1C[C@H](O)C[C@H]1C(=O)NCc1ccc(-c2cnco2)cc1)C(C)(C)C]

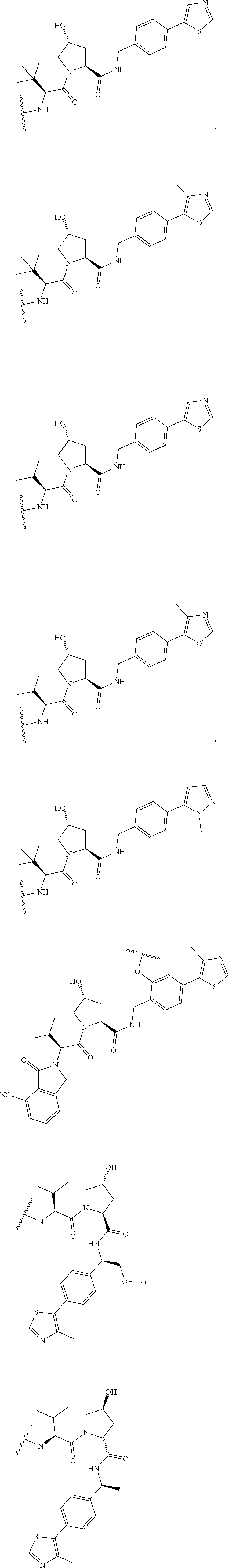

wherein

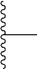

indicates where the PLM attaches to the Linker LI.

In some embodiments of the compound of formula (Q), the the PLM is selected from:

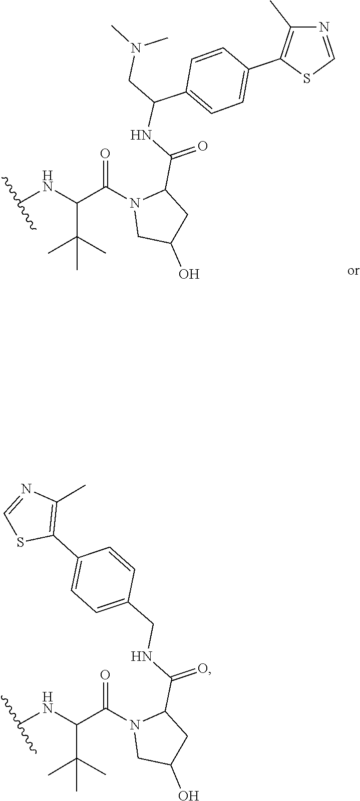 [SMILES: *NC(C(=O)N1CC(O)CC1C(=O)NC(CN(C)C)c1ccc(-c2scnc2C)cc1)C(C)(C)C ; *NC(C(=O)N1CC(O)CC1C(=O)NCc1ccc(-c2scnc2C)cc1)C(C)(C)C]

wherein

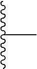

indicates where the PLM attaches to the Linker LI.

In some embodiments of the compound of formula (Q), the the PLM is selected from

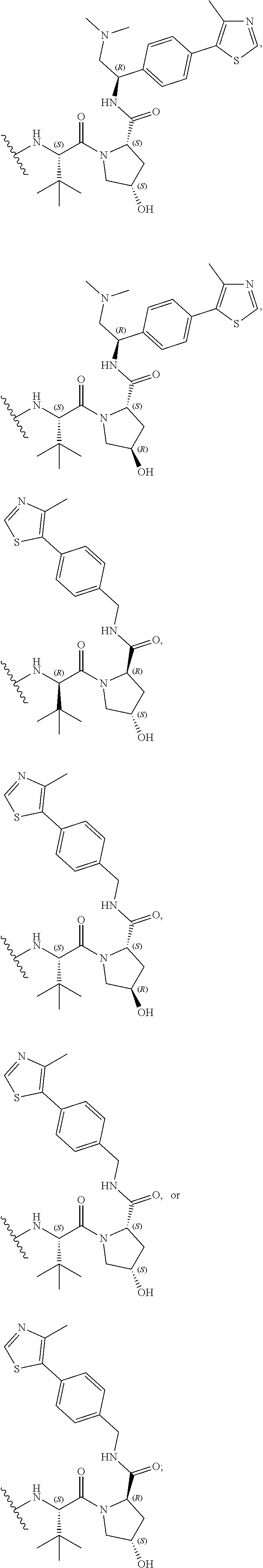

wherein the PLM is covalently bound to the LI via

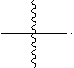

In one embodiment, the PLM has the structure of formula (E3D2)

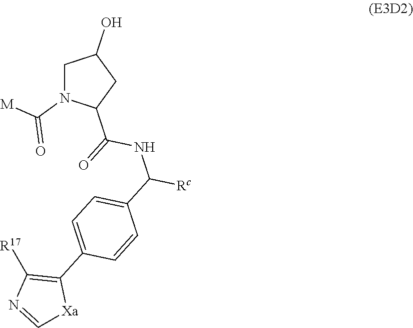

wherein Xa is O or S

R^{c} is H, methyl or ethyl

R¹⁷ is H, methyl, ethyl, hydoxymethyl or cyclopropyl;

M is optionally substituted heteroaryl, optionally substituted aryl or —CR⁹R¹⁰R¹¹;

R⁹ is H;

R¹⁰ is H, optionally substituted alkyl, optionally substituted haloalkyl, optionally substituted heteroaryl, optionally substituted aryl, optionally substituted hydroxyalkyl, optionally substituted thioalkyl or cycloalkyl;

R¹¹ is optionally substituted heteroaromatic, optionally substituted heterocyclic, optionally substituted aryl or —NR¹²R¹³;

R¹² is H or optionally substituted alkyl;

R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate; and wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI.

In some embodiments, any one of the hydrogen atoms in the PLM can be replaced to form a covalent bond to the LI.

In one embodiment, the PLM is selected from

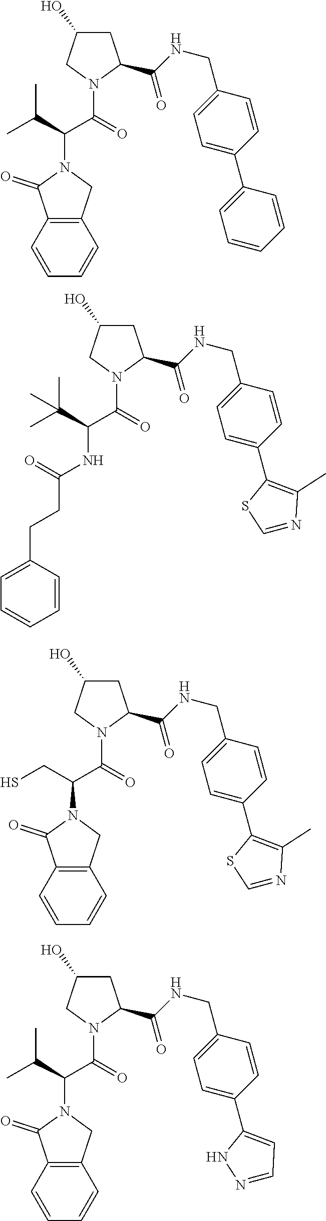

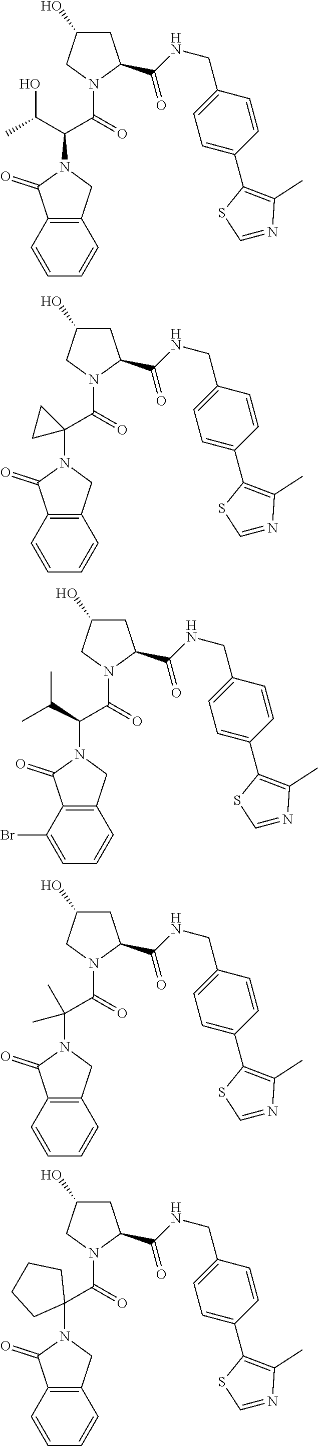

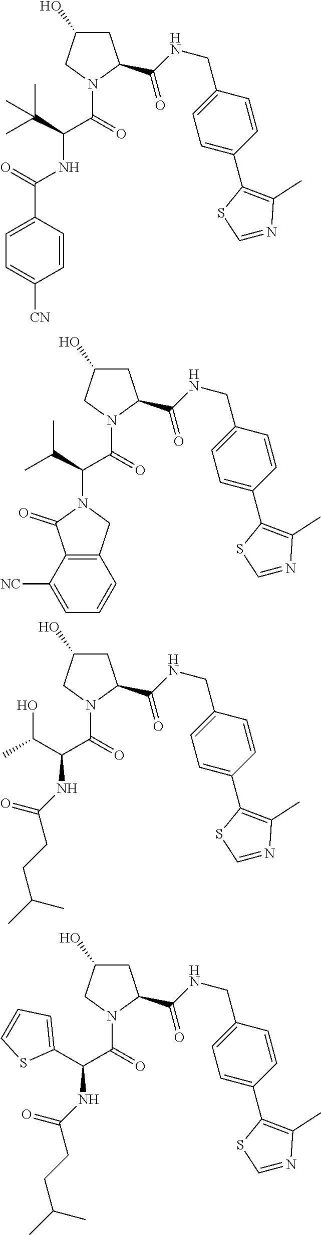

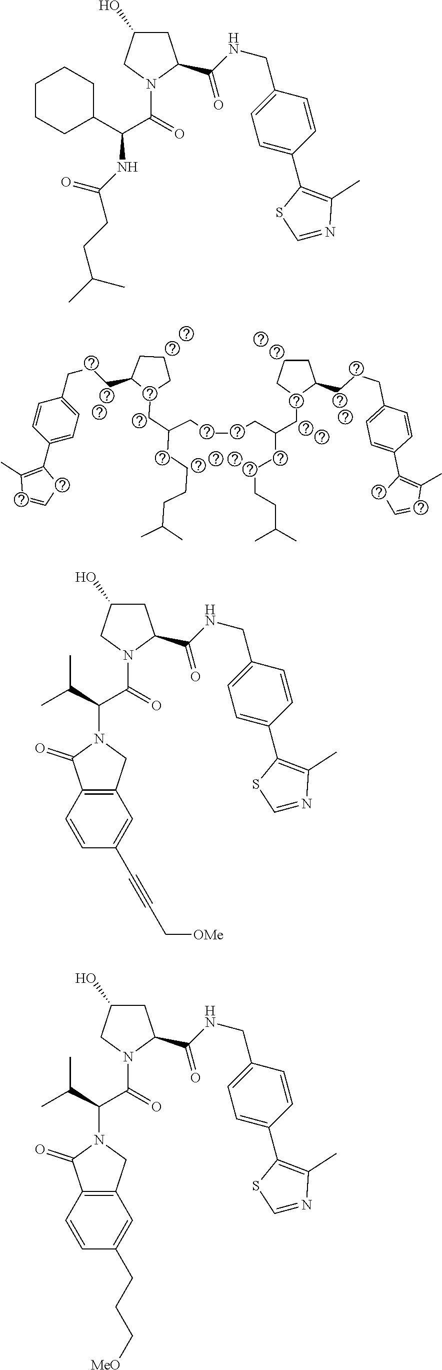

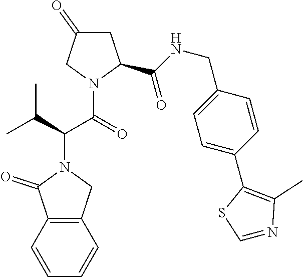 [SMILES: Cc1ncsc1-c1ccc(CNC(=O)[C@@H]2CC(=O)CN2C(=O)[C@H](C(C)C)N2Cc3ccccc3C2=O)cc1]

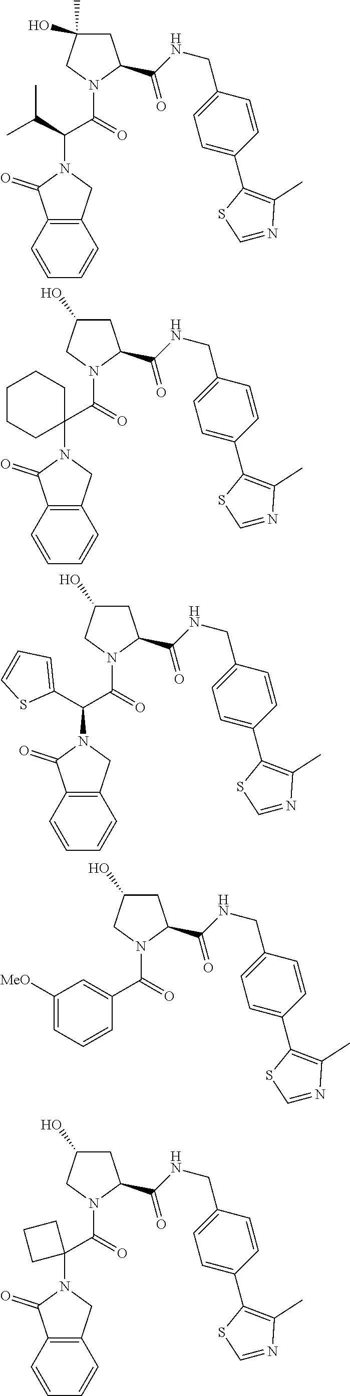

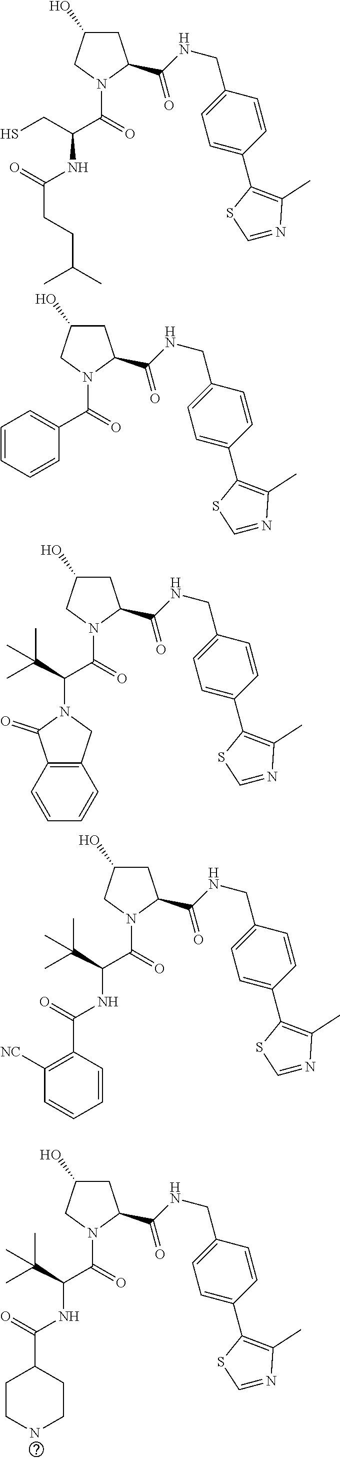

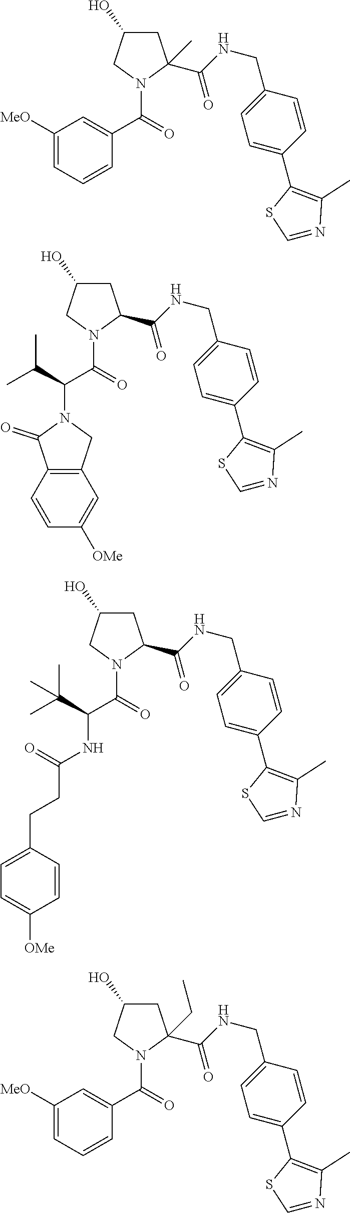

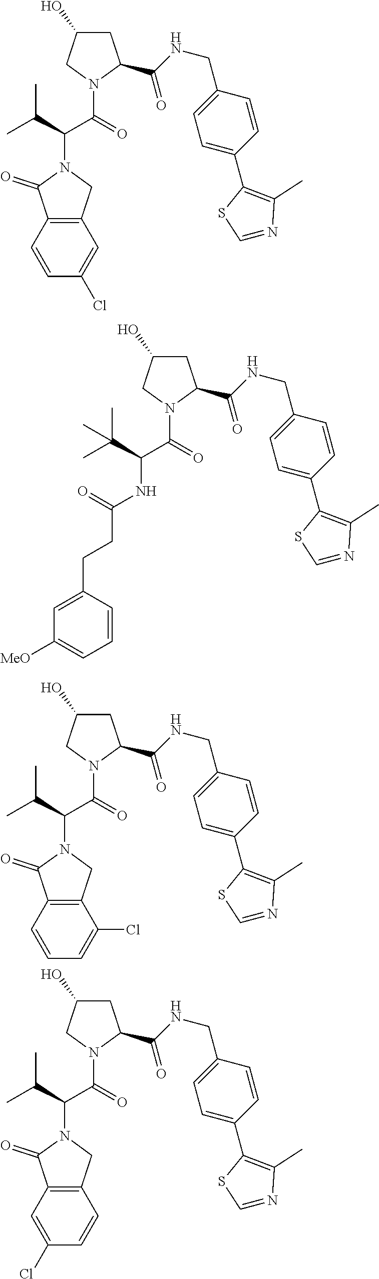

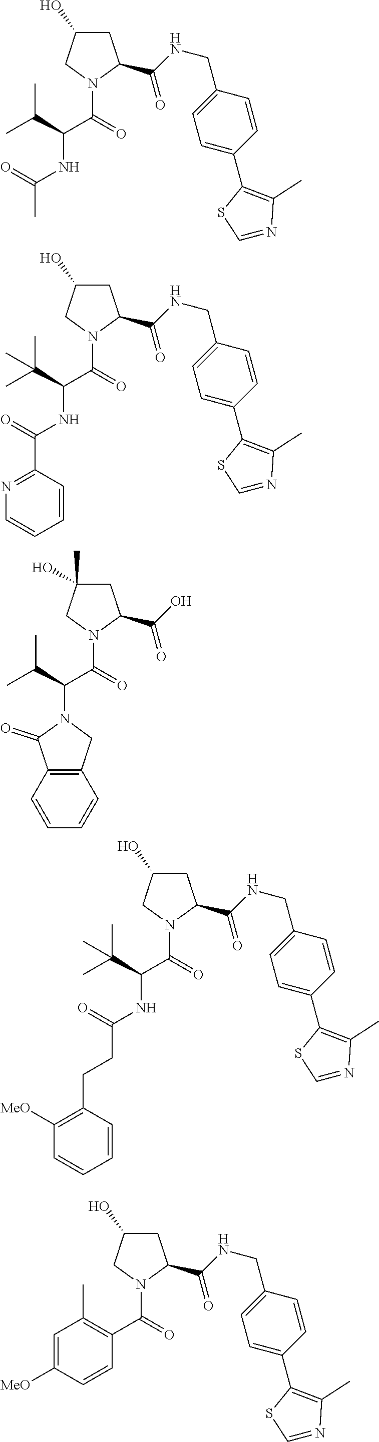

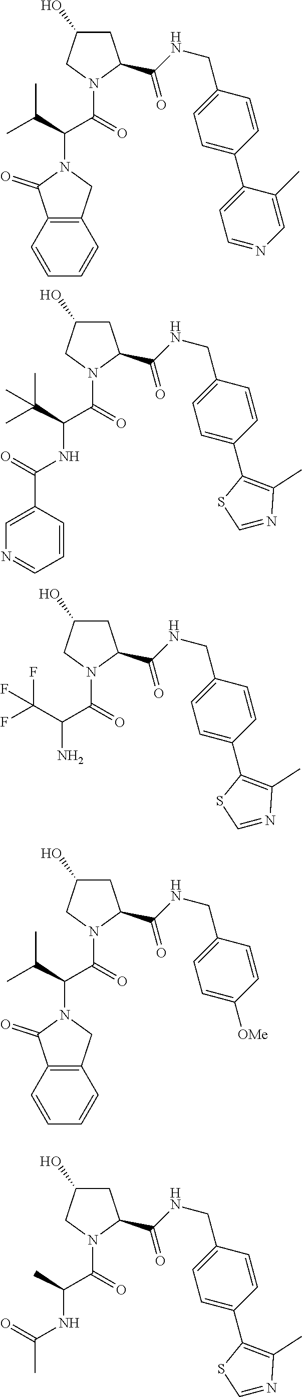

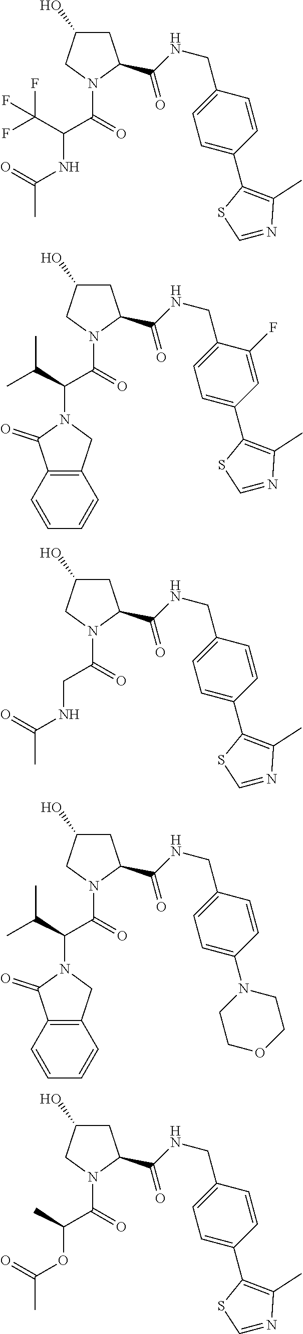

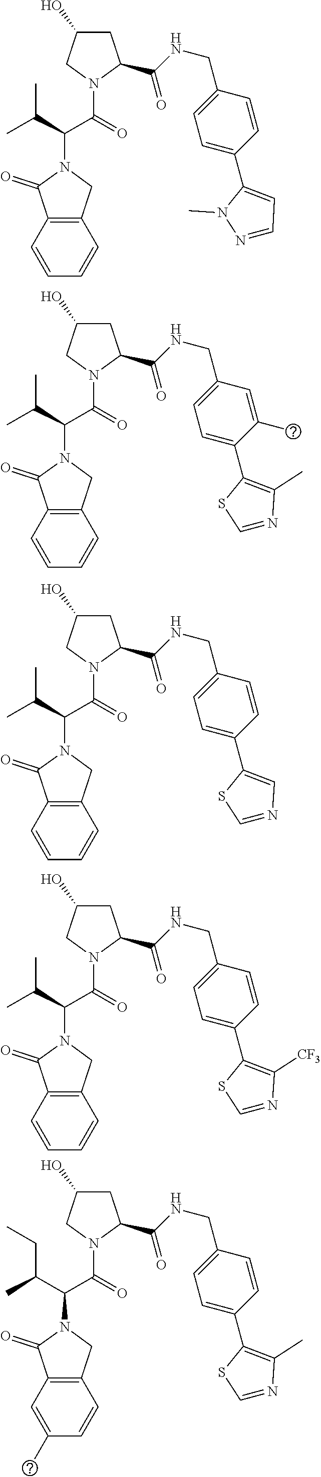

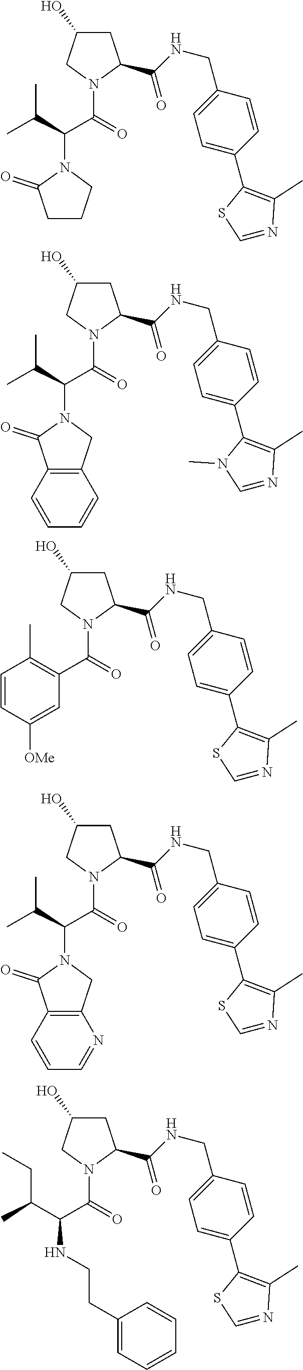

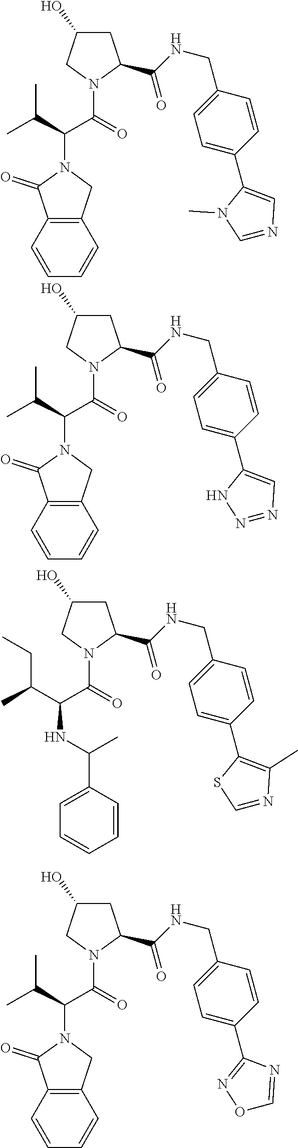

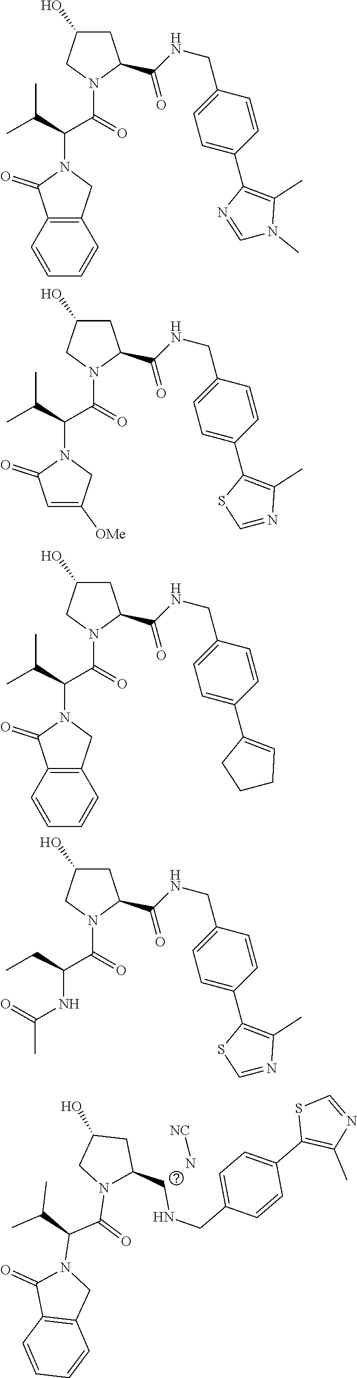

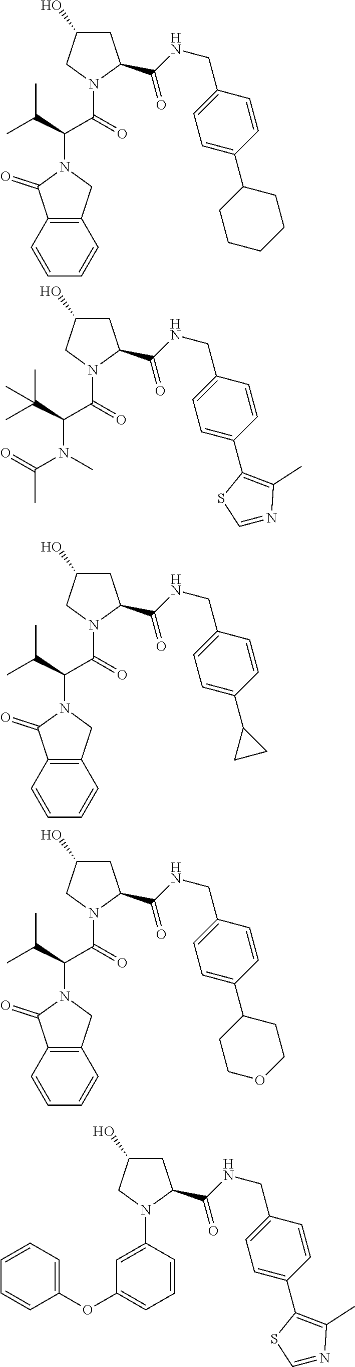

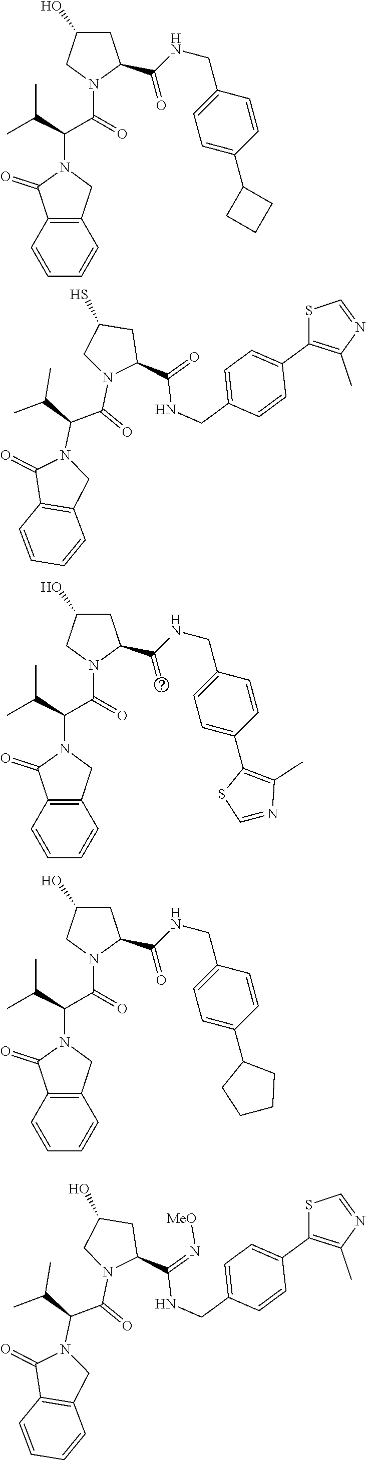

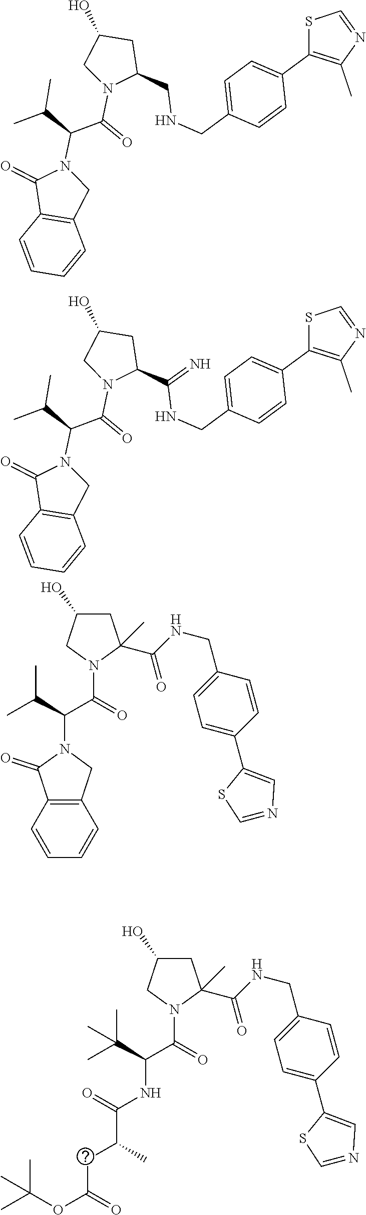

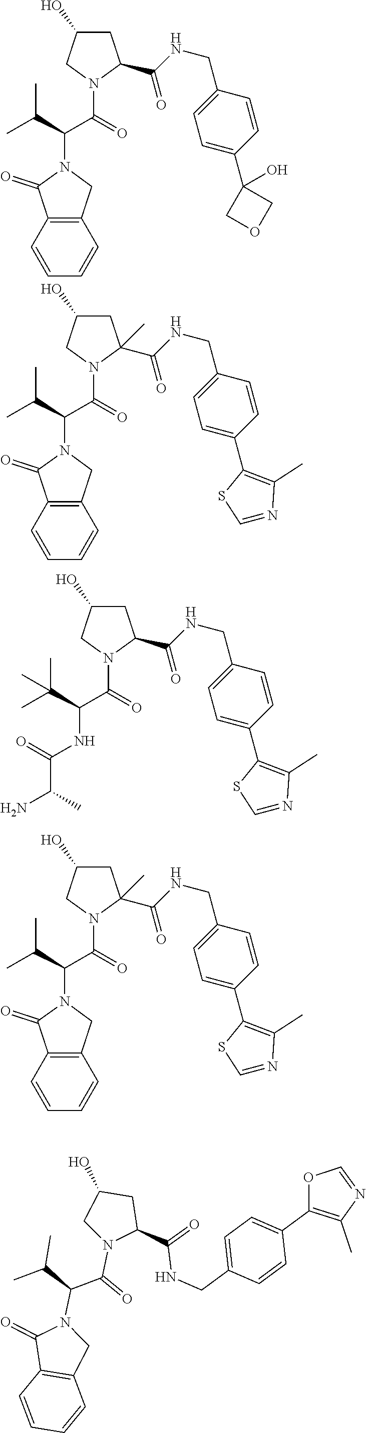

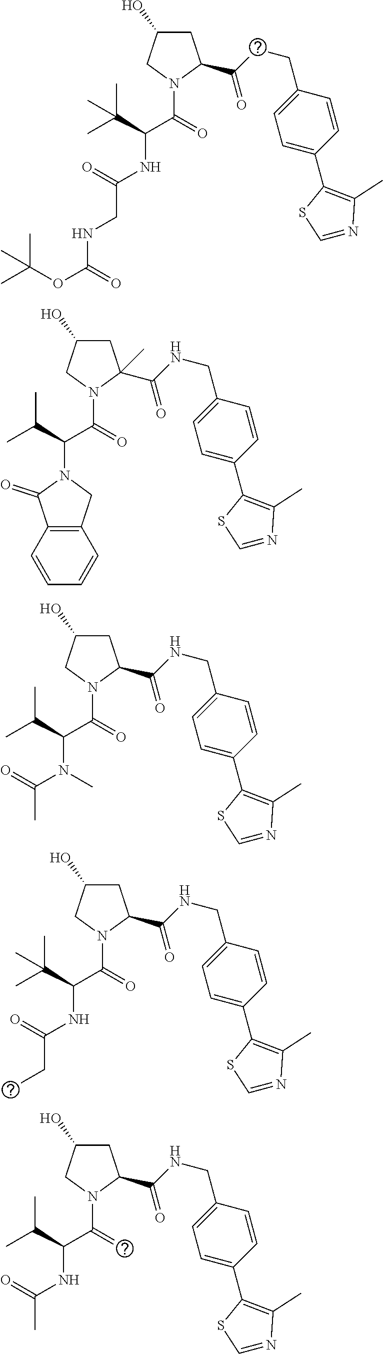

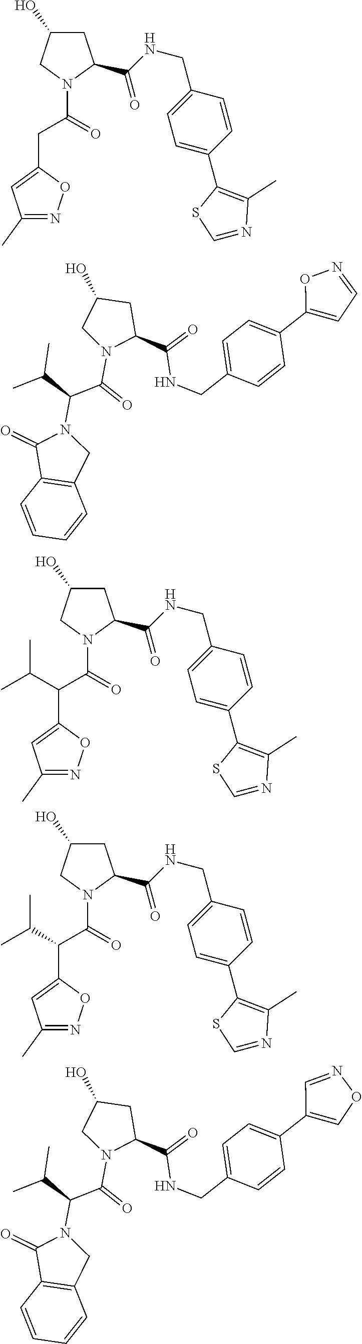

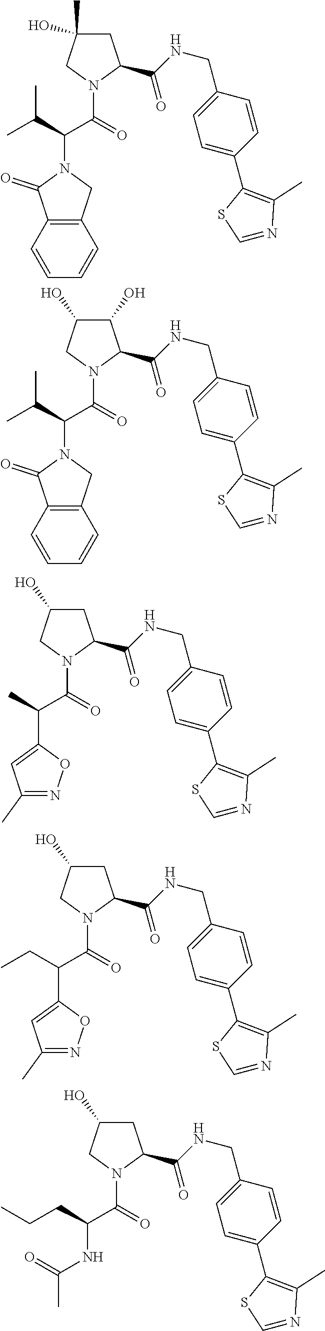

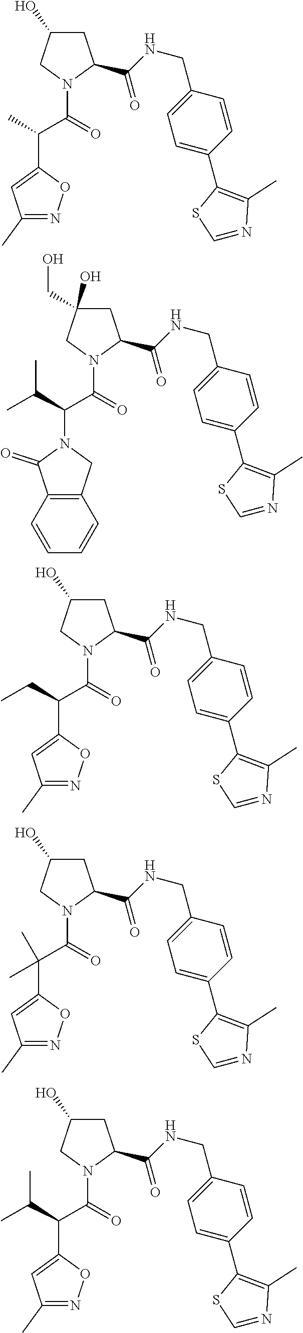

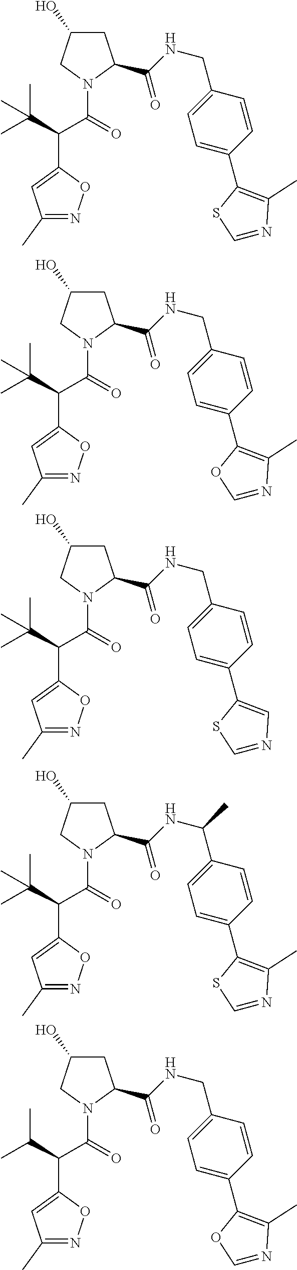

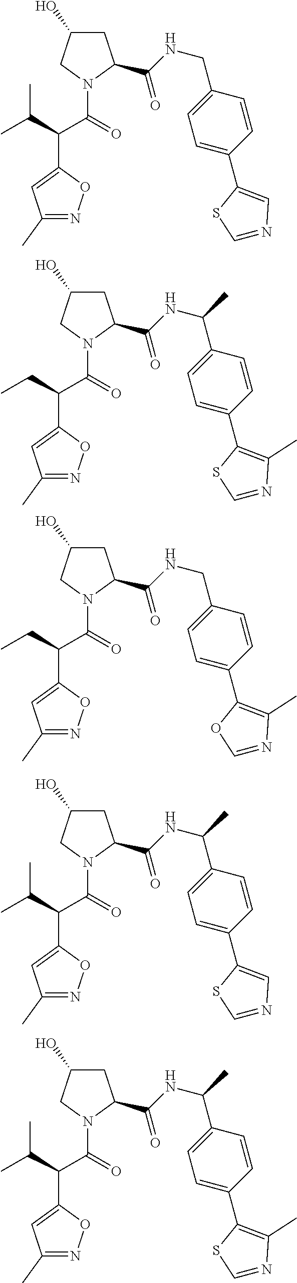

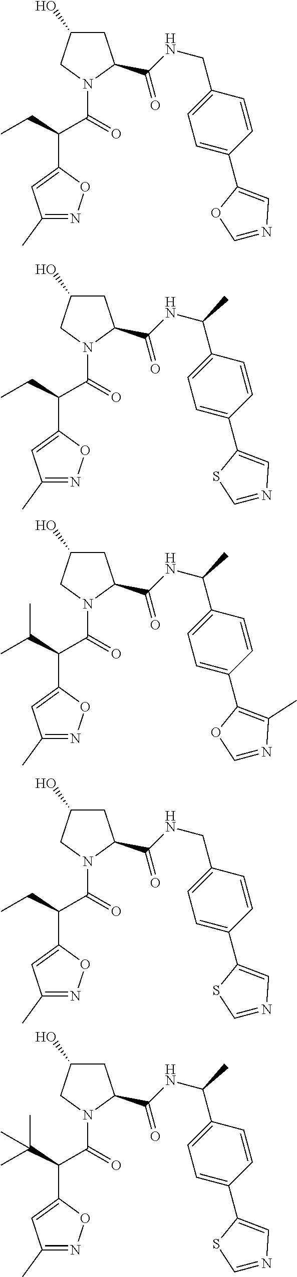

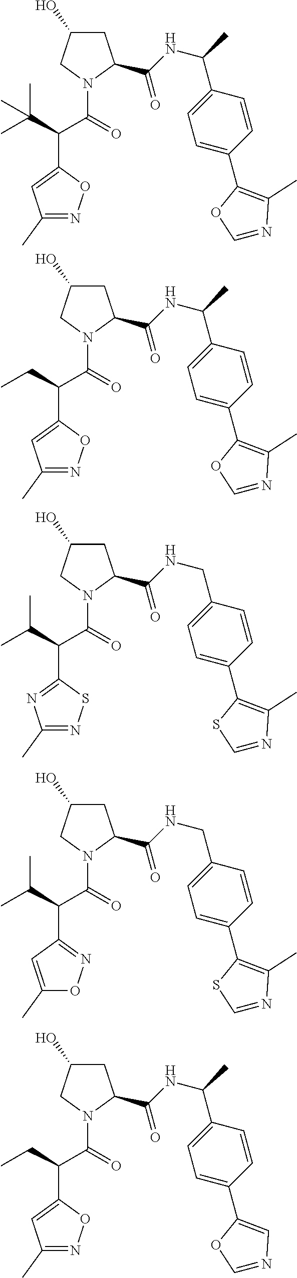

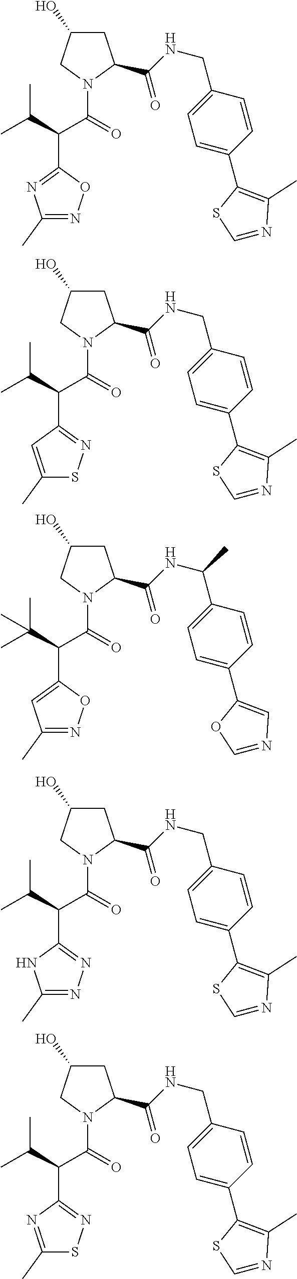

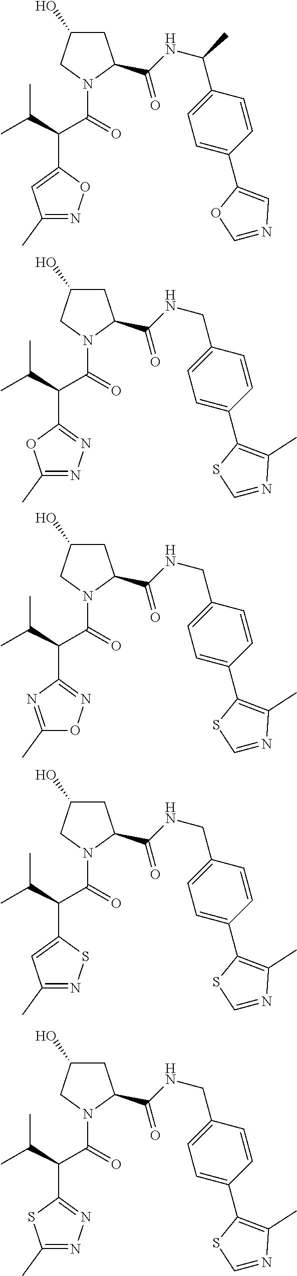

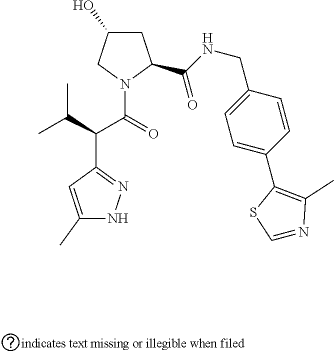

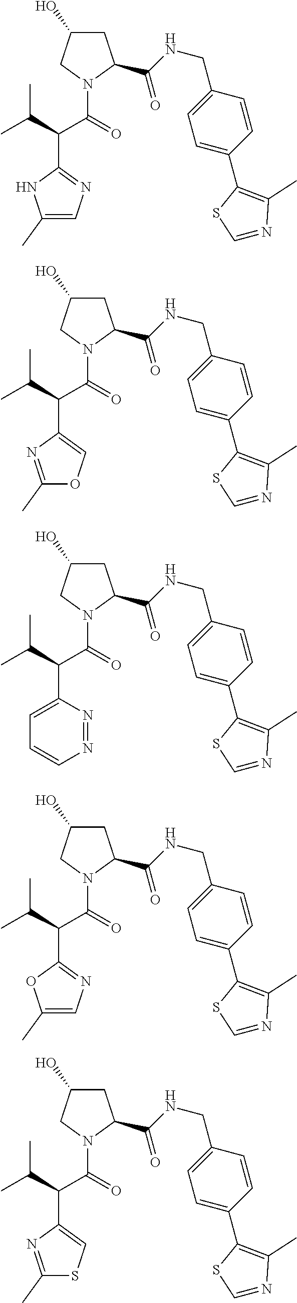

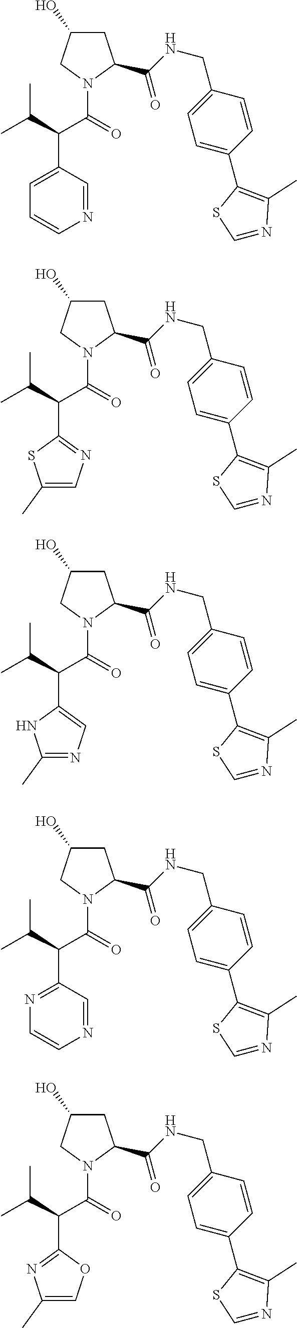

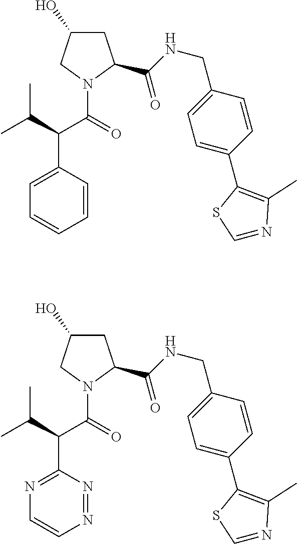

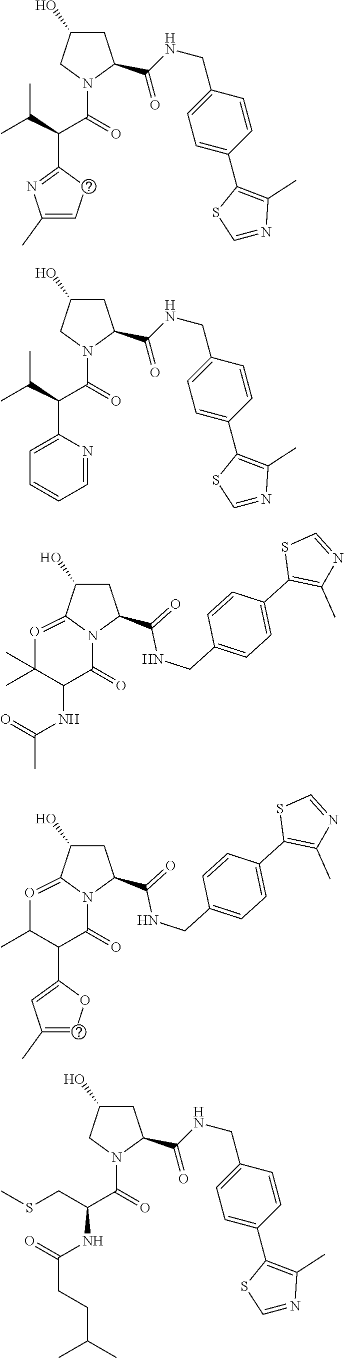

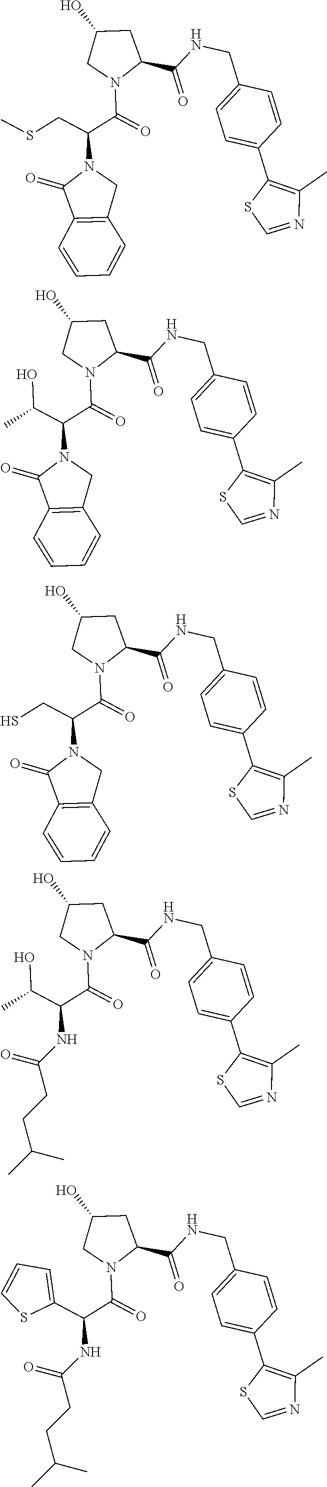

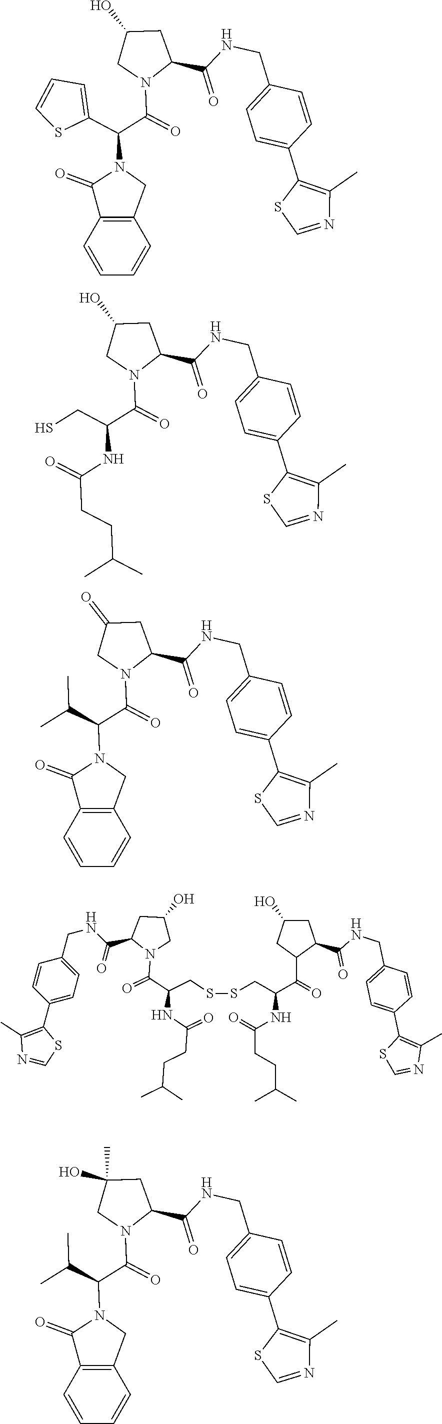

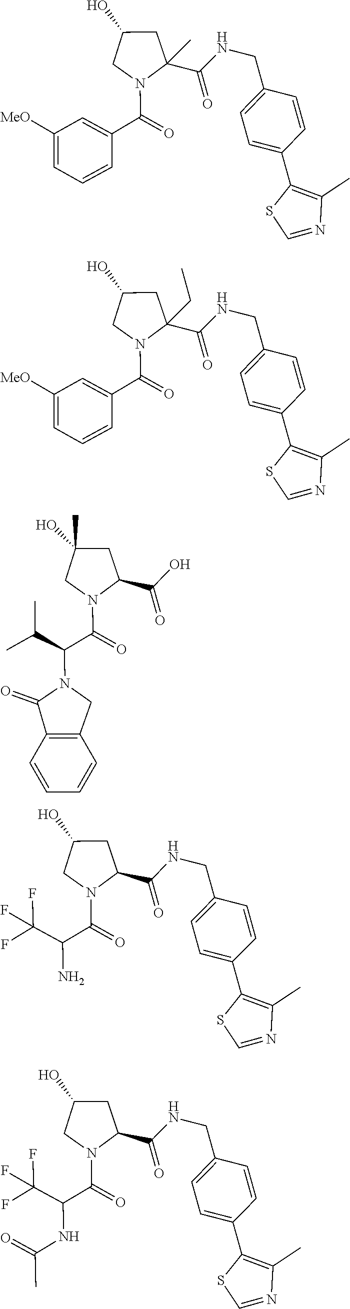

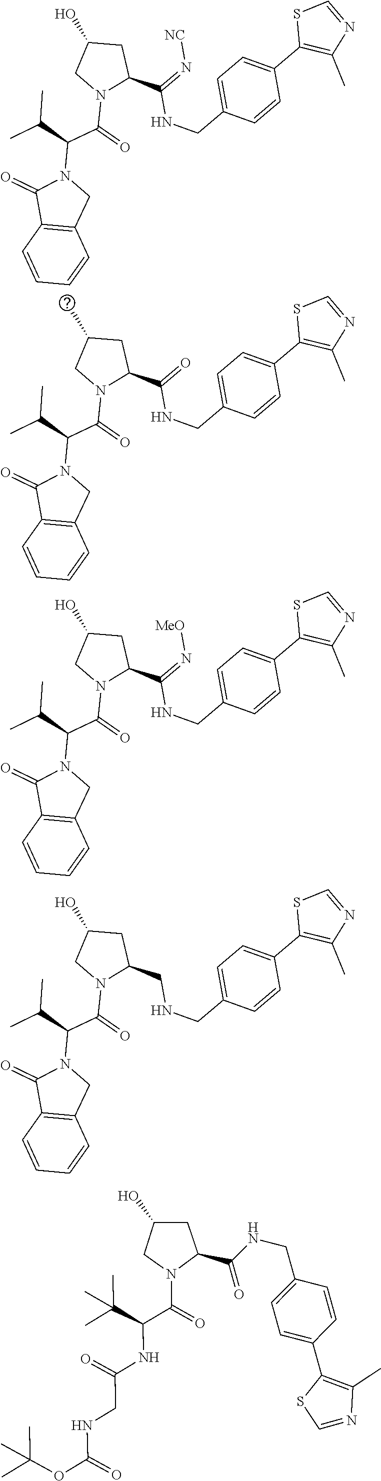

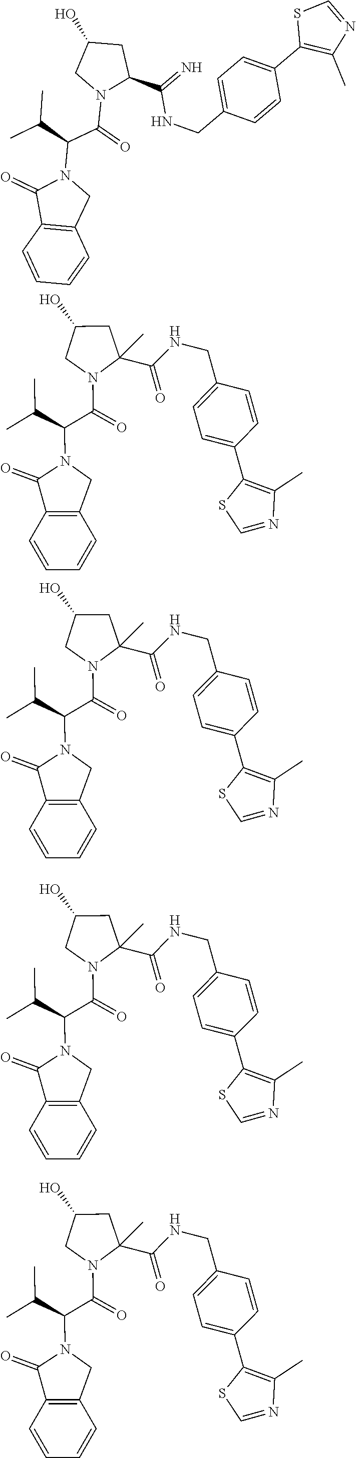

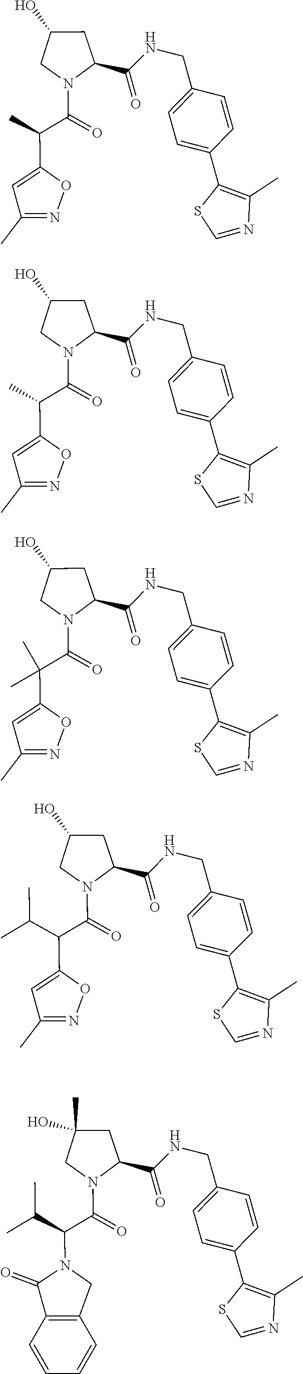

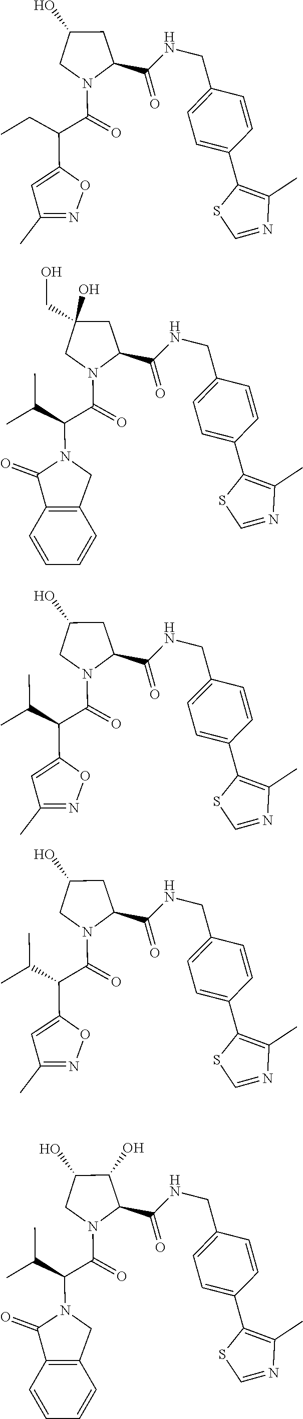

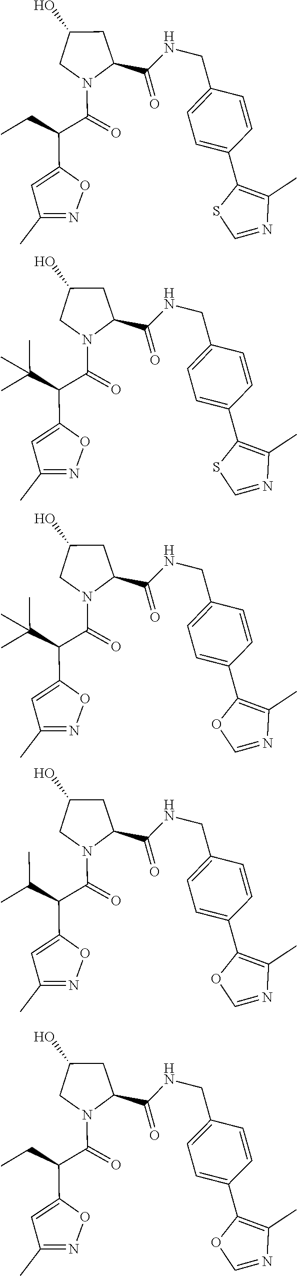

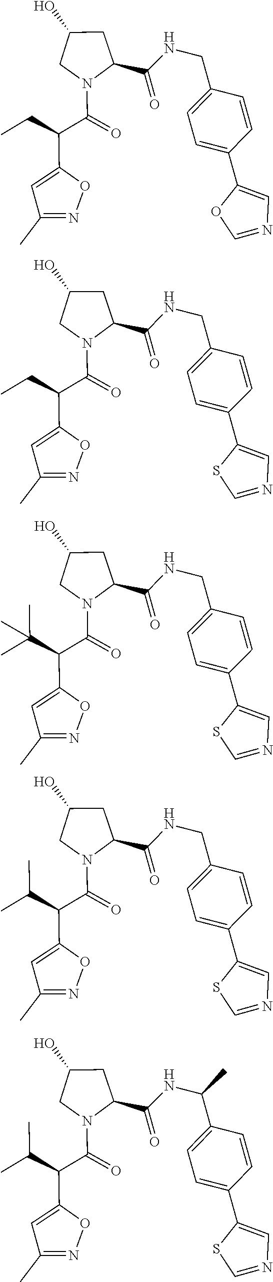

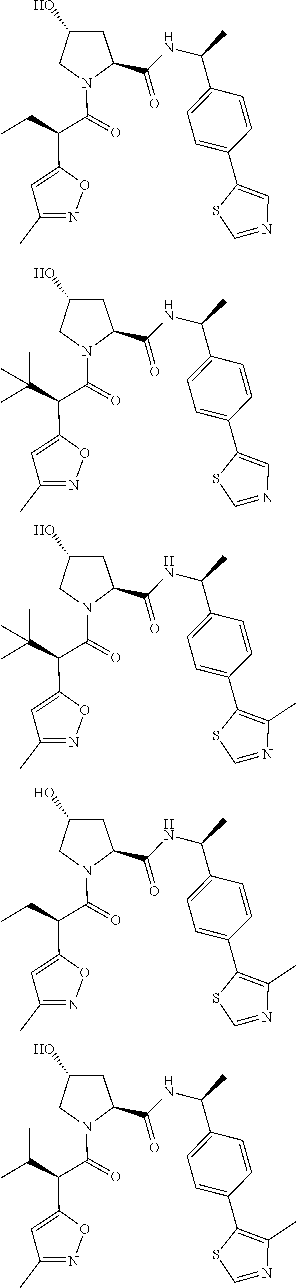

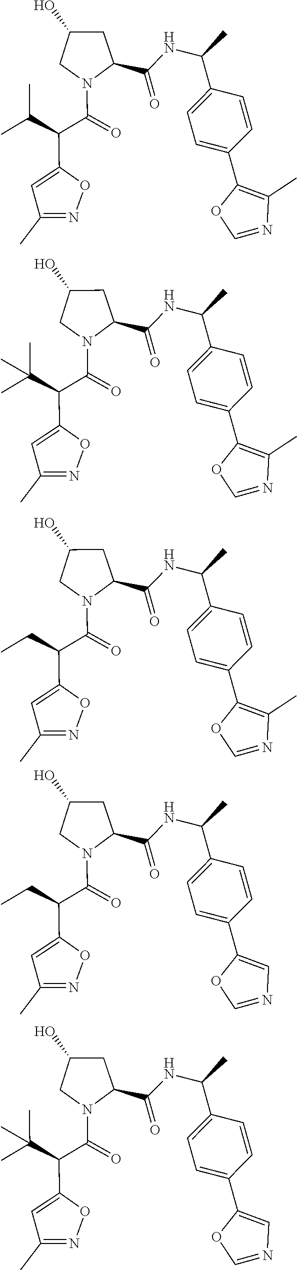

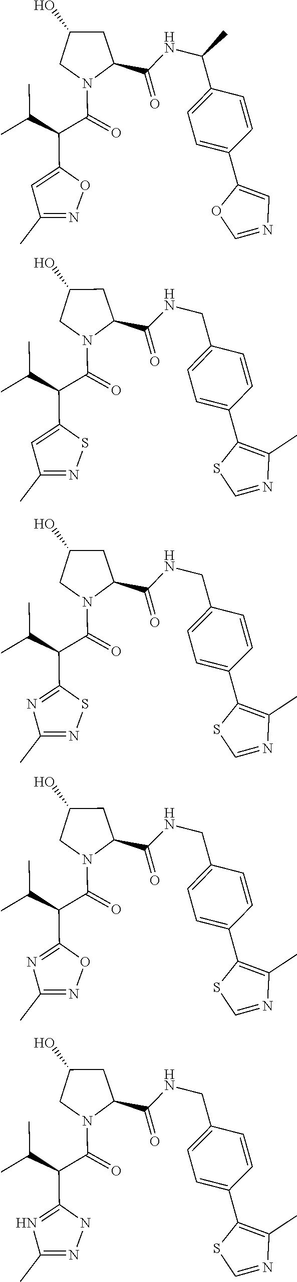

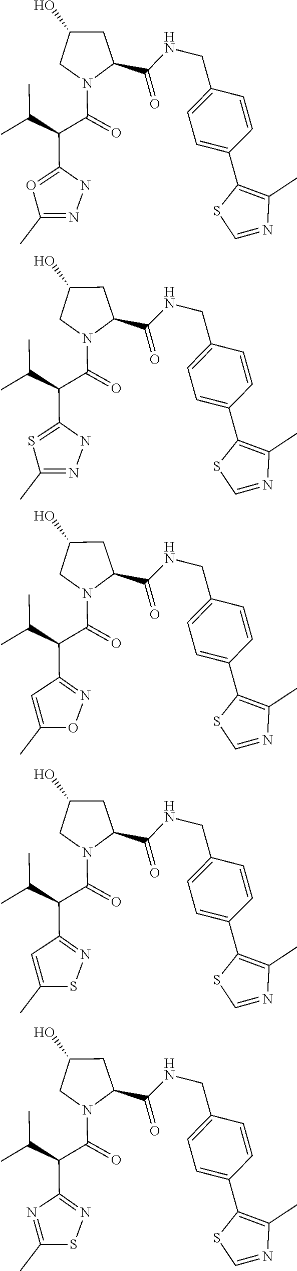

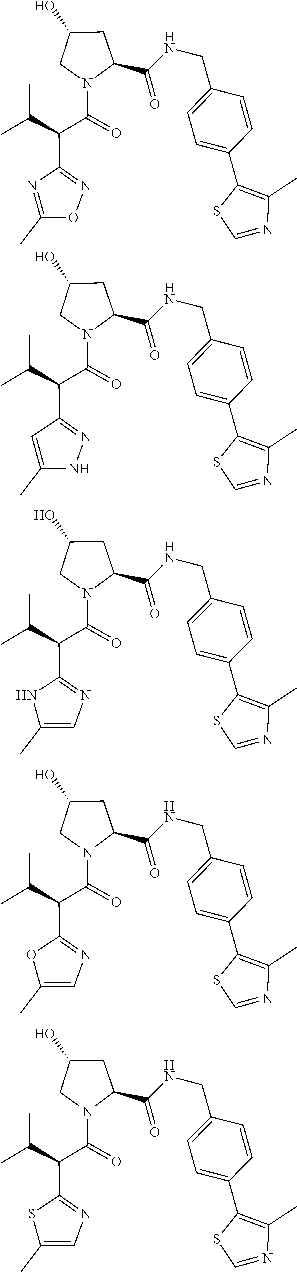

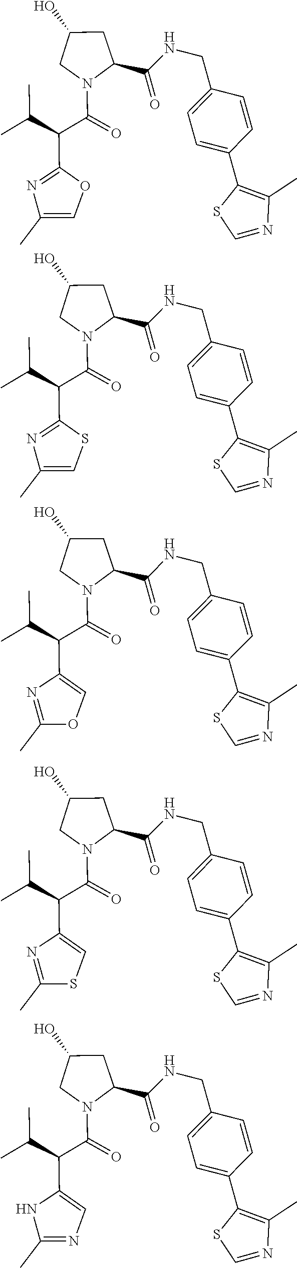

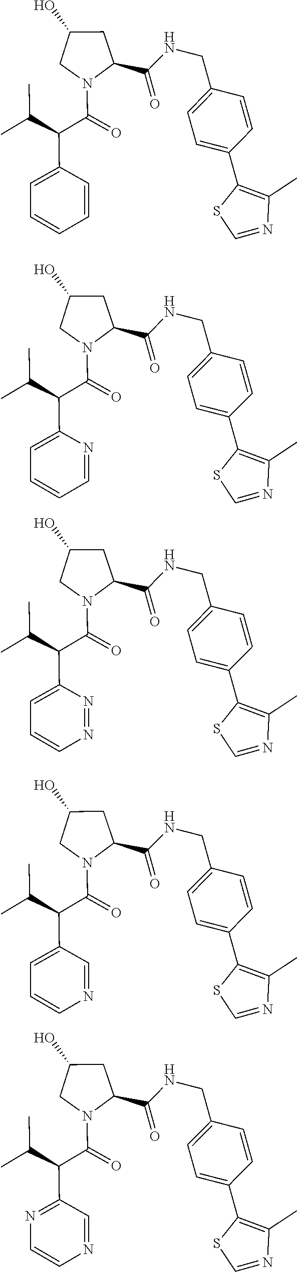

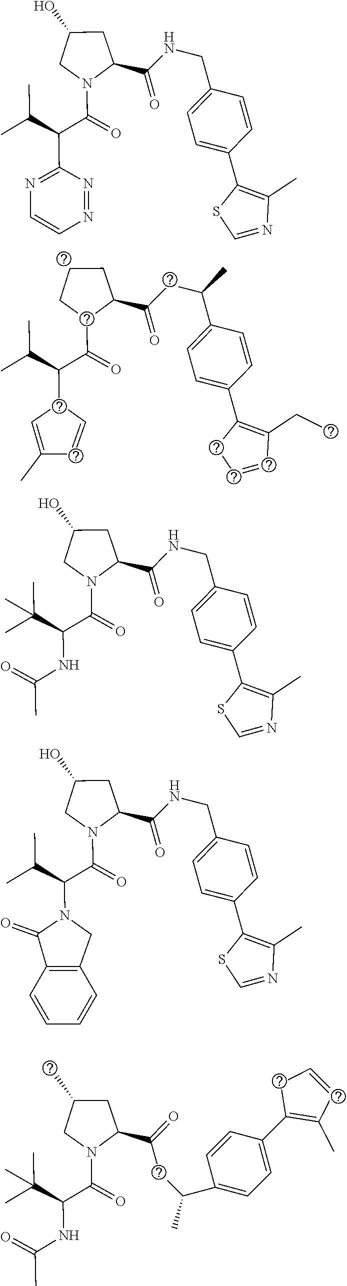

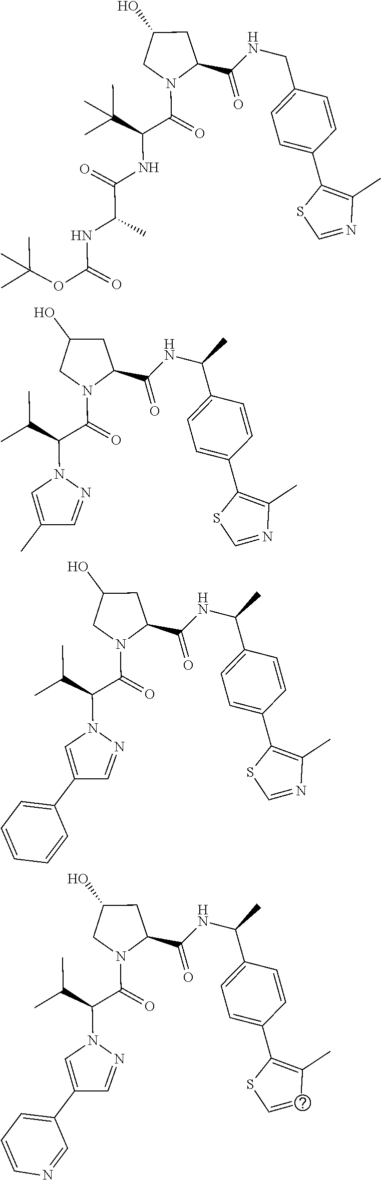

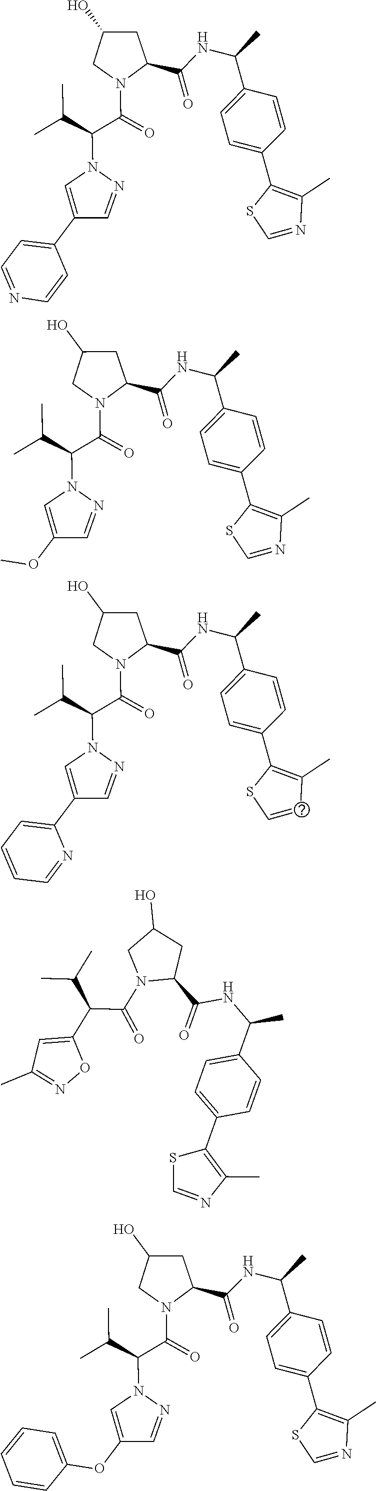

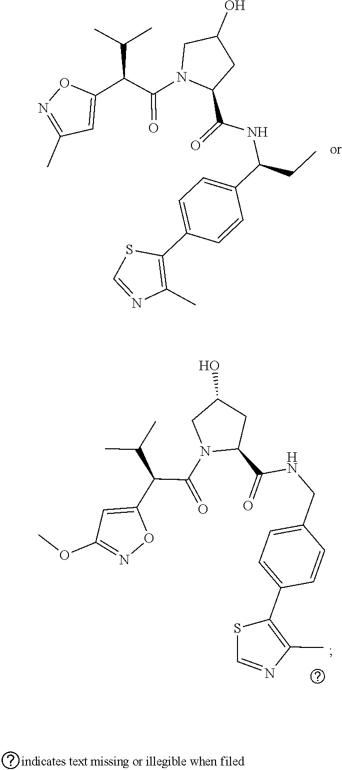

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H, O—H), or halogen is used to form the covalent bond between PLM and the linker LI. In some embodiments, the hydrogen from a N—H group is replaced to form the covalent bond between PLM and the linker LI.

In one embodiment, the PLM is selected from

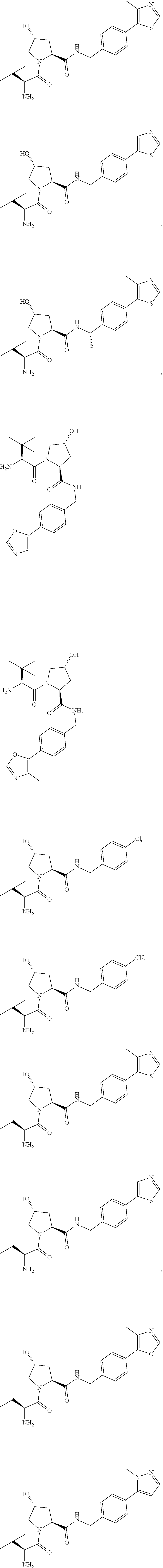

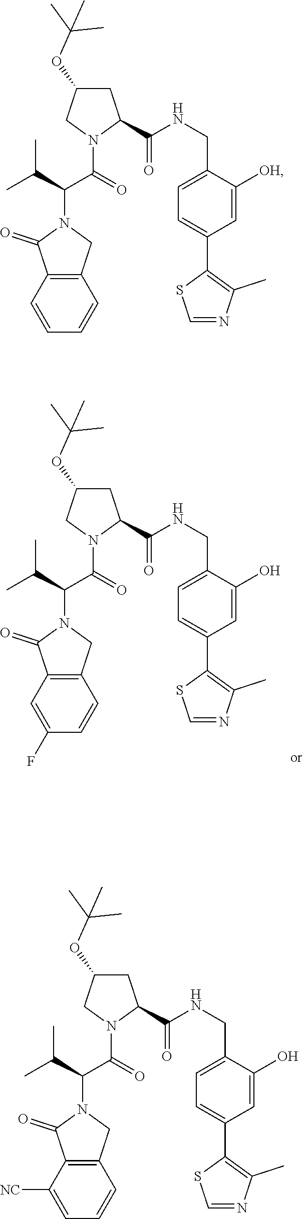 [SMILES: Cc1ncsc1-c1ccc(CNC(=O)[C@@H]2C[C@@H](OC(C)(C)C)CN2C(=O)[C@H](C(C)C)N2Cc3ccccc3C2=O)c(O)c1 ; Cc1ncsc1-c1ccc(CNC(=O)[C@@H]2C[C@@H](OC(C)(C)C)CN2C(=O)[C@H](C(C)C)N2Cc3ccc(F)cc3C2=O)c(O)c1 ; Cc1ncsc1-c1ccc(CNC(=O)[C@@H]2C[C@@H](OC(C)(C)C)CN2C(=O)[C@H](C(C)C)N2Cc3cccc(C#N)c3C2=O)c(O)c1]

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H, O—H), or halogen is used to form the covalent bond between PLM and the linker LI. In some embodiments, the hydrogen from a N—H group is replaced to form the covalent bond between PLM and the linker LI.

In some embodiments of the compound of formula (Q), the PLM is represented by formula (W-IIIA):

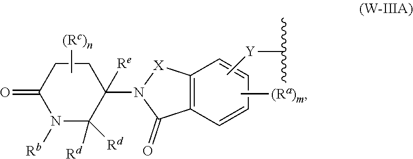

or an enantiomer, diastereomer, stereoisomer, or a pharmaceutically acceptable salt thereof, wherein:
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently halogen, OH, C₁₋₆ alkyl, or C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O), a C₃-C₆ carbocycle, or a 4- to 6-membered heterocycle comprising 1 or 2 heteroatoms selected from N or O;
R^{e} is H, deuterium, C₁₋₃ alkyl, F, or Cl;
m is 0, 1, 2 or 3;
n is 0, 1 or 2; and
wherein the PLM is covalently bound to the LI via

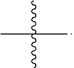

In some embodiments of the compound of formula (Q), the PLM is represented by formula (W-IIIB):

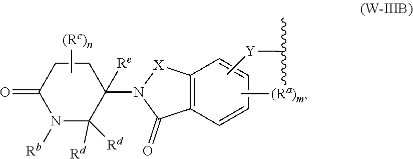

or an enantiomer, diastereomer, stereoisomer, or a pharmaceutically acceptable salt thereof, wherein:

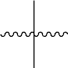

represent a bond to the LI;
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR or;
X is —C(O)— or —C(R^{b})₂—;
each R^{a} is independently C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O) or a C₃-C₆ carbocycle;
R^{e} is H, deuterium, C₁₋₃ alkyl, F, or Cl;
m is 0, 1, 2 or 3;
n is 0, 1 or 2; and
wherein the PLM is covalently bound to the LI via

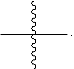

In some embodiments of the PLM of formula (W-IIIA) or formula (W-IIIB), X is —C(C₁₋₃ alkyl)₂.

In some embodiments of the compound of formula (Q), the PLM is selected from the group consisting of:

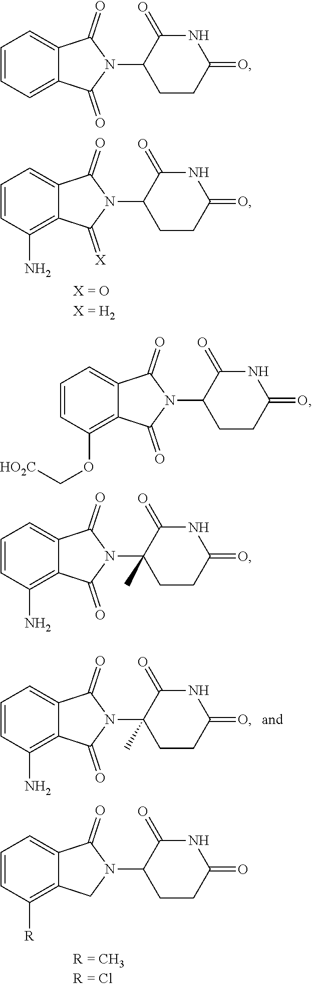

wherein any one of the hydrogen atoms in the PLM can be replaced to form a covalent bond to the LI.

In some embodiments of the compound of formula (Q), the PLM is:

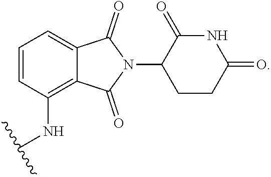 [SMILES: *Nc1cccc2c1C(=O)N(C1CCC(=O)NC1=O)C2=O]

In one embodiment, the PLM is selected from

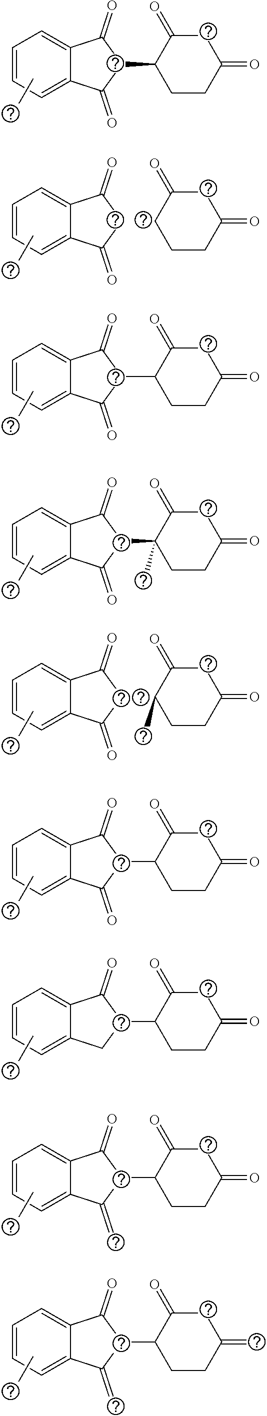

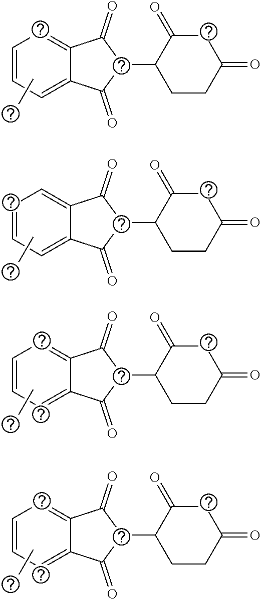

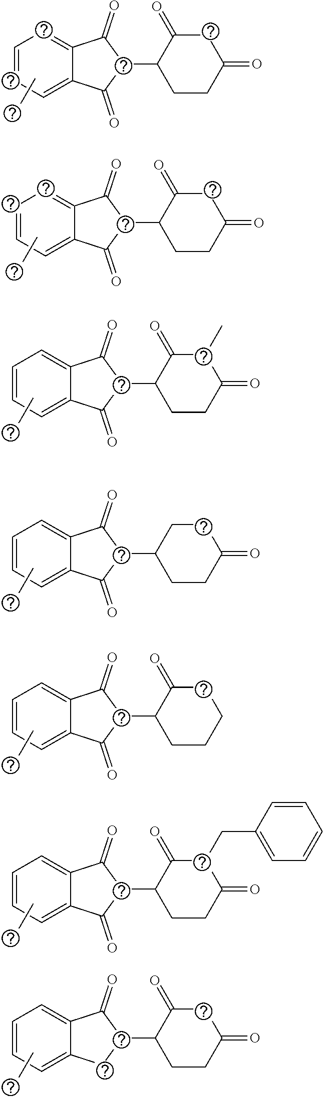

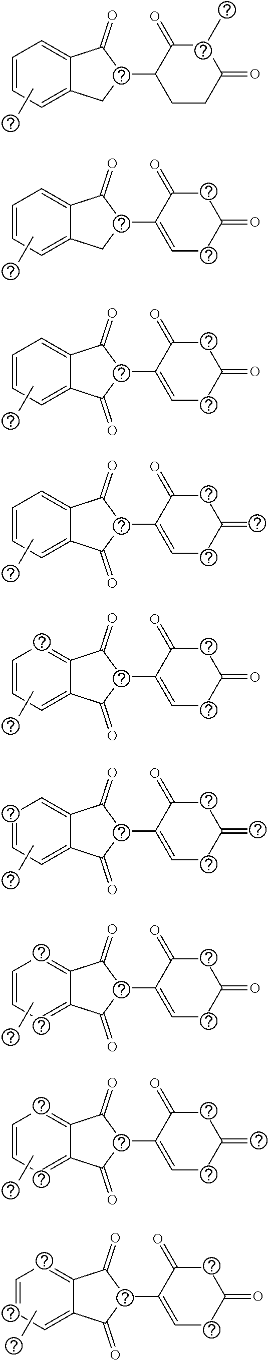

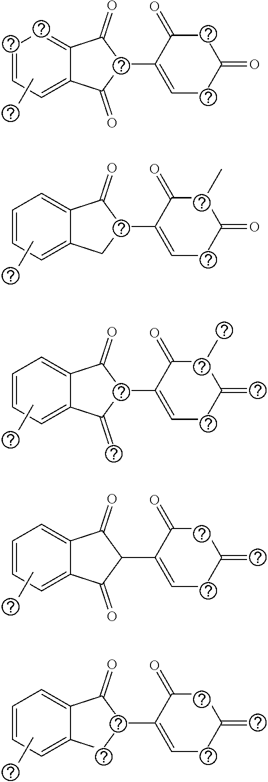

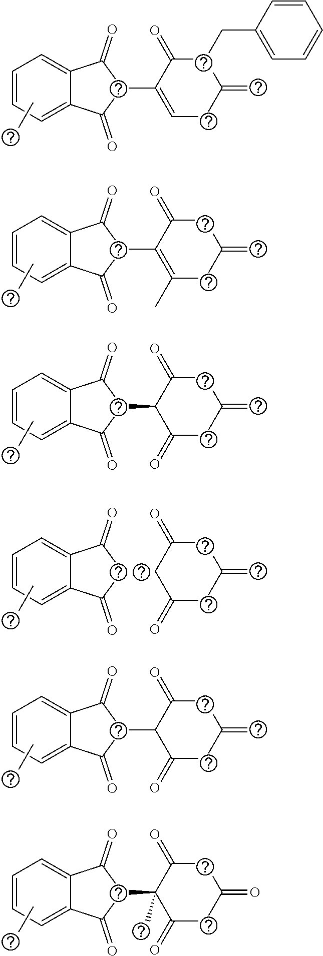

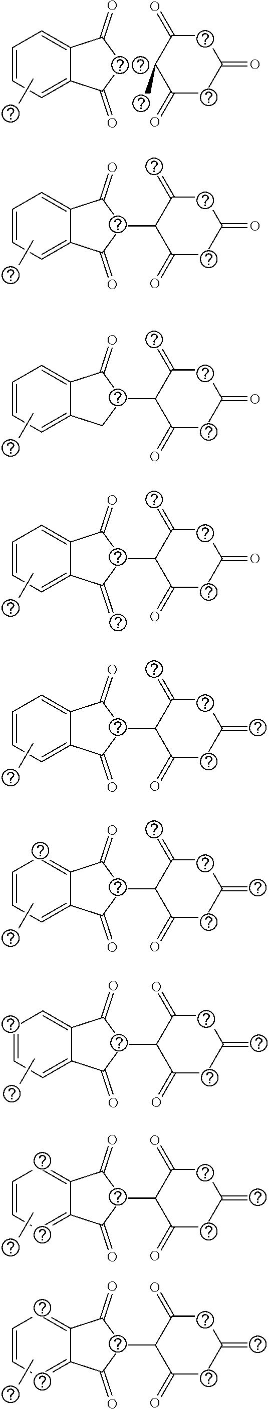

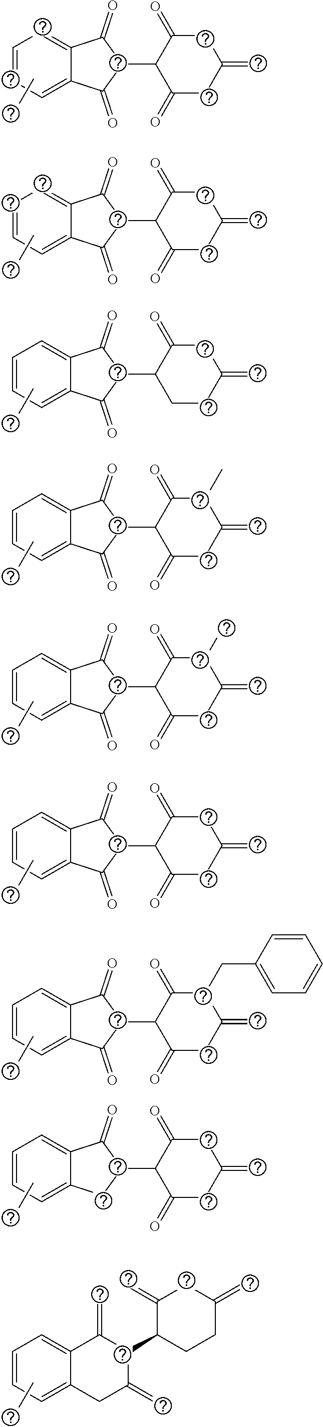

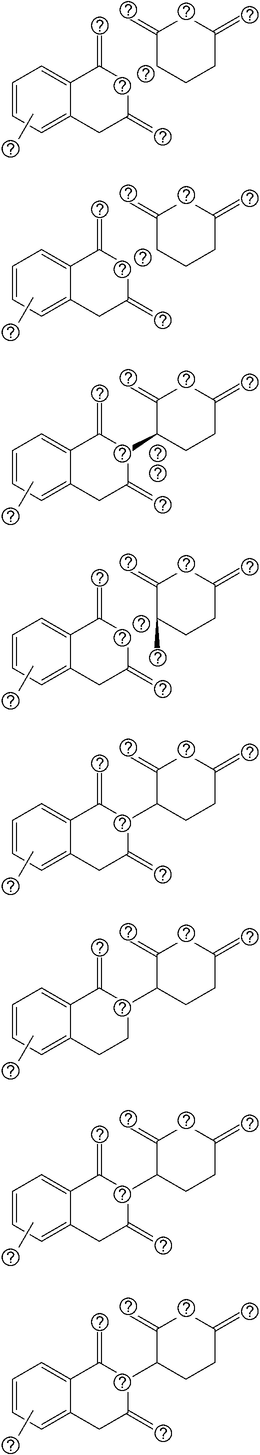

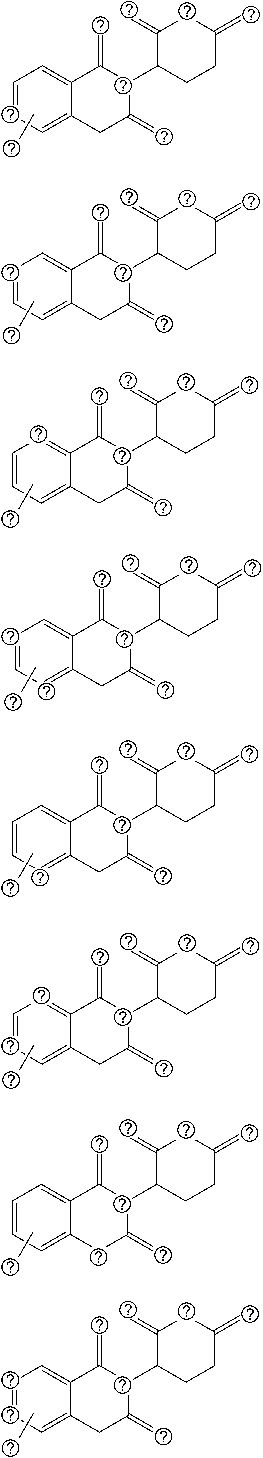

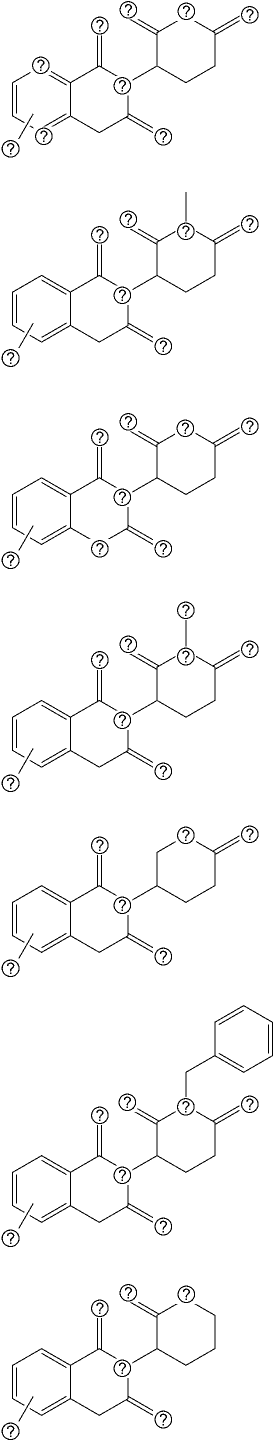

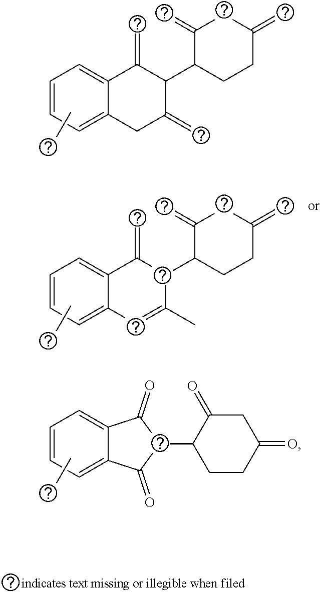

wherein R is a functional group or an atom, and optionally one of which can be modified to be covalently joined to a Linker LI and n is 1, 2, 3, or 4.

In one embodiment, the PLM has the structure of formula (E3Ga)-(E3Gd):

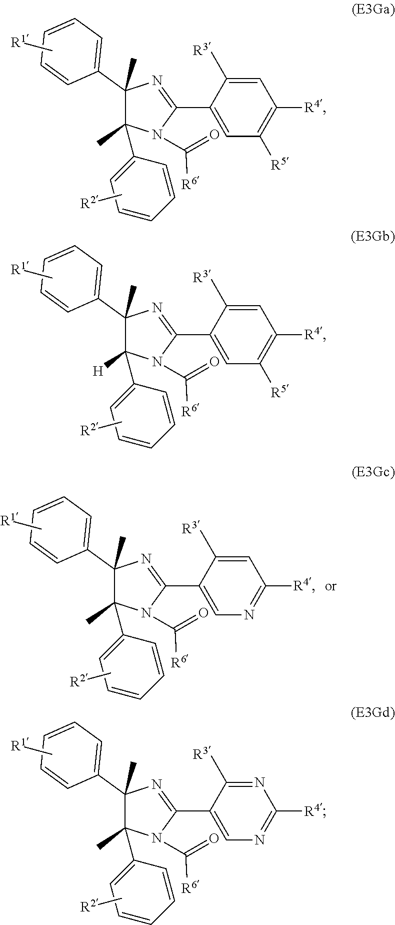

wherein R^{1′} and R^{2′} are independently selected from the group consisting of F, Cl, Br, I, acetylene, CN, CF₃ and NO₂;

R^{3′} is selected from the group consisting of —OCH₃, —OCH₂CH₃, —OCH₂CH₂F, —OCH₂CH₂OCH₃, and —OCH(CH₃)₂;

R^{4′} is selected from the group consisting of H, halogen, —CH₃, —CF₃, —OCH₃, —C(CH₃)₃, —CH(CH₃)₂, -cyclopropyl, —CN, —C(CH₃)₂OH, —C(CH₃)₂OCH₂CH₃, —C(CH₃)₂CH₂OH, —C(CH₃)₂CH₂OCH₂CH₃, —C(CH₃)₂CH₂OCH₂CH₂OH, —C(CH₃)₂CH₂OCH₂CH₃, —C(CH₃)₂CN, —C(CH₃)₂C(O)CH₃, —C(CH₃)₂C(O)NHCH₃, —C(CH₃)₂C(O)N(CH₃)₂, —SCH₃, —SCH₂CH₃, —S(O)₂CH₃, —S(O₂)CH₂CH₃, ˜NHC(CH₃)₃, —N(CH₃)₂, pyrrolidinyl, and 4-morpholinyl;

R^{5′} is selected from the group consisting of halogen, -cyclopropyl, —S(O)₂CH₃, —S(O)₂CH₂CH₃, 1-pyrrodinyl, —NH₂, —N(CH₃)₂, and —NHC(CH₃)₃; and

R^{6′} is selected from the structures presented below where the linker LI connection point is indicated by *

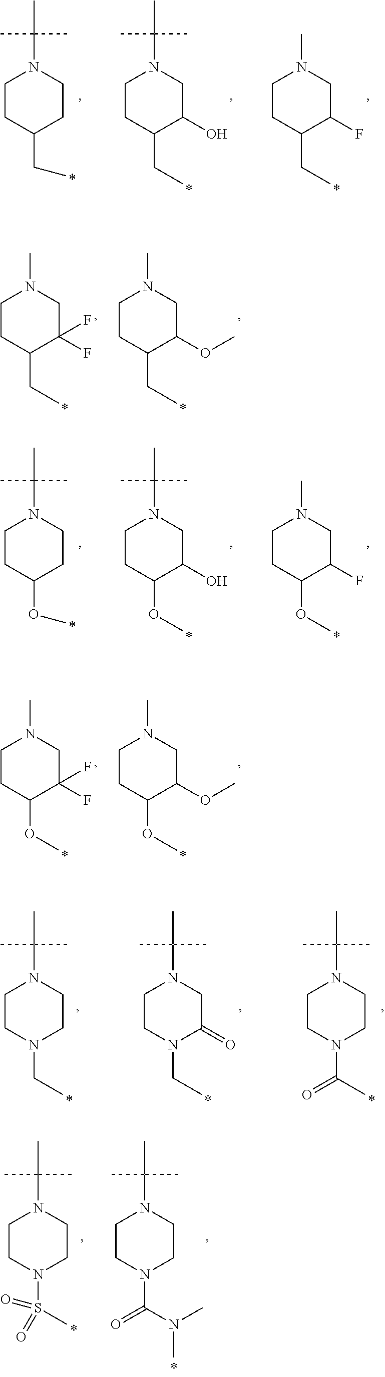

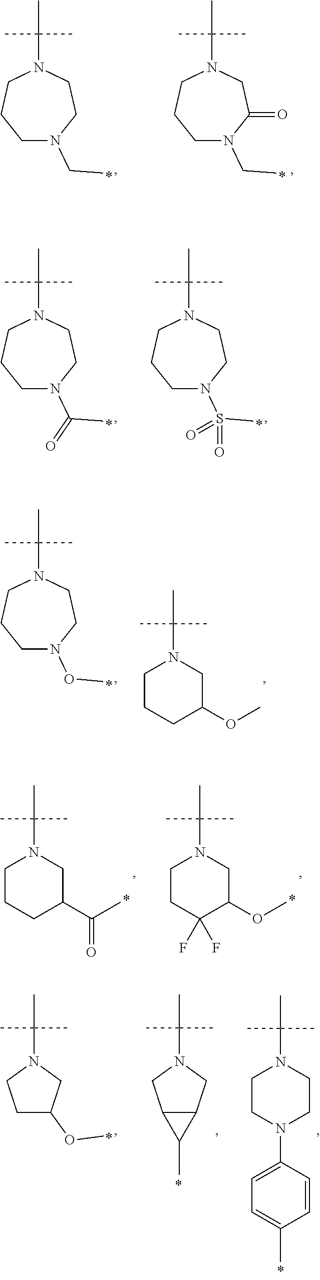

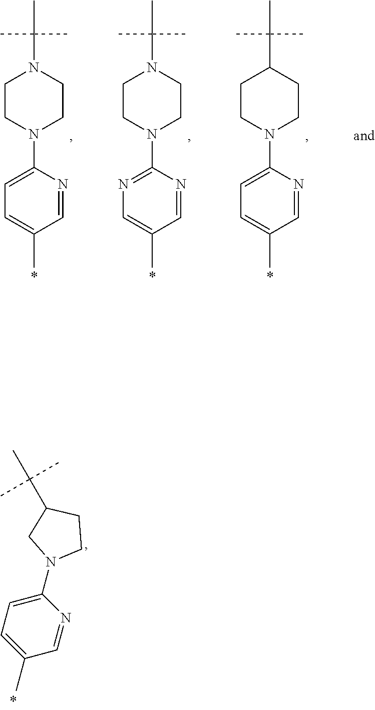

In one embodiment of the PLM has the structure of formula (E3Ga)-(E3Gd), beside R^{6′} as the point for linker attachment, R^{4′} can also serve as the linker attachment position. In the case that R^{4′} is the linker connection site, the linker will be connected to the terminal atom of R^{4′} groups.

In one embodiment, the PLM is selected from

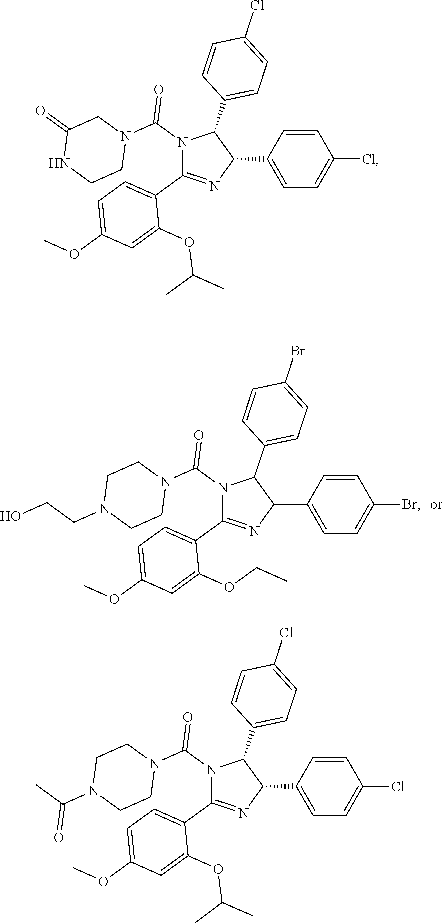 [SMILES: COc1ccc(C2=N[C@@H](c3ccc(Cl)cc3)[C@@H](c3ccc(Cl)cc3)N2C(=O)N2CCNC(=O)C2)c(OC(C)C)c1 ; CCOc1cc(OC)ccc1C1=NC(c2ccc(Br)cc2)C(c2ccc(Br)cc2)N1C(=O)N1CCN(CCO)CC1 ; COc1ccc(C2=N[C@@H](c3ccc(Cl)cc3)[C@@H](c3ccc(Cl)cc3)N2C(=O)N2CCN(C(C)=O)CC2)c(OC(C)C)c1]

and wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H, O—H), or halogen is used to form the covalent bond between PLM and the linker LI. In some embodiments, the hydrogen from a N—H group is replaced to form the covalent bond between PLM and the linker LI.

In one embodiment, the PLM is selected from

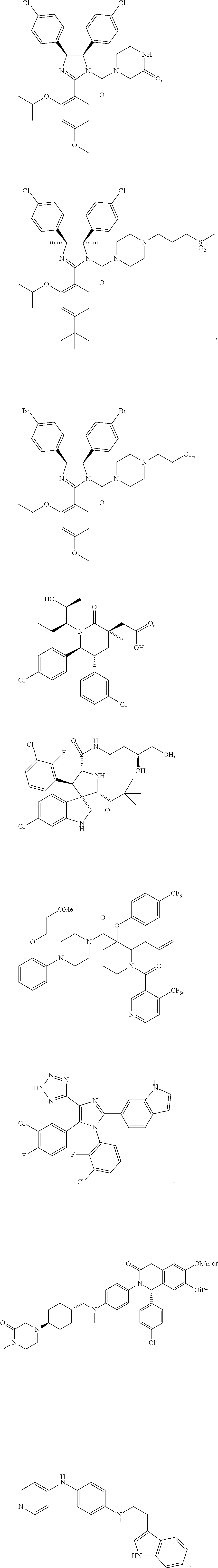

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H, O—H), or halogen is used to form the covalent bond between PLM and the linker LI. In some embodiments, the hydrogen from a N—H group is replaced to form the covalent bond between PLM and the linker LI. In some embodiments of the compound of formula (Q), the PLM is

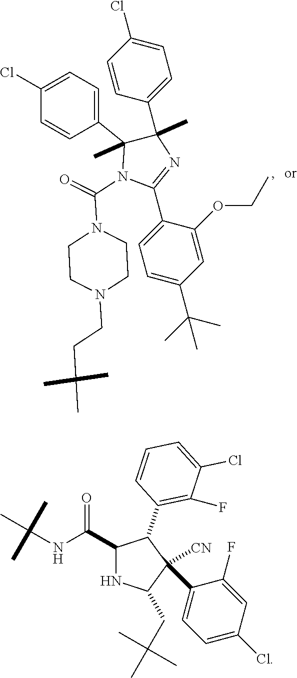

In one embodiment, the PLM comprises an alanine-valine-proline-isoleucine tetrapeptide fragment or an unnatural mimetic thereof.

In one embodiment, the PLM is selected from

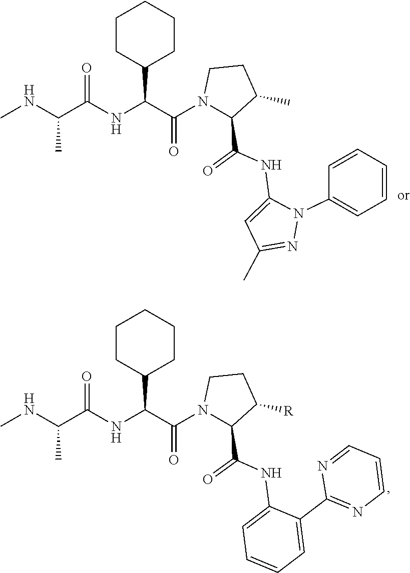

wherein R is H or methyl; wherein any one of the hydrogen atoms in the PLM can be replaced to form a covalent bond to the LI. In some embodiments, the hydrogen from a N—H group is replaced to form the covalent bond between PLM and the linker LI.

In one embodiment, the PLM is selected from

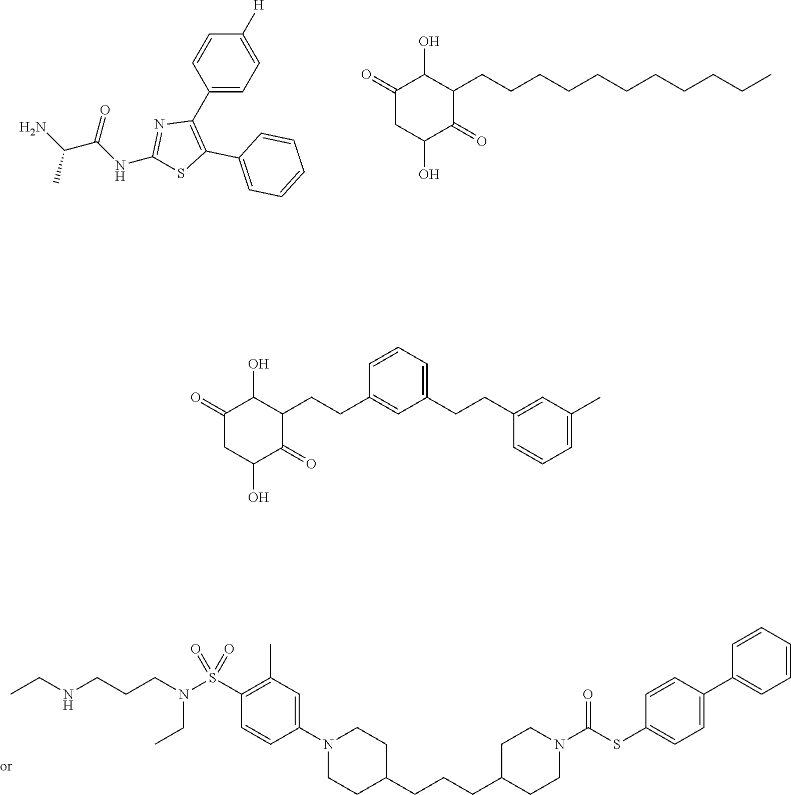 [SMILES: C[C@H](N)C(=O)Nc1nc(-c2ccccc2)c(-c2ccccc2)s1 ; CCCCCCCCCCCC1C(=O)C(O)CC(=O)C1O ; Cc1cccc(CCc2cccc(CCC3C(=O)C(O)CC(=O)C3O)c2)c1 ; CCNCCCN(CC)S(=O)(=O)c1ccc(N2CCC(CCCC3CCN(C(=O)Sc4ccc(-c5ccccc5)cc4)CC3)CC2)cc1C]

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H, O—H), or halogen is used to form the covalent bond between PLM and the linker LI. In some embodiments, the hydrogen from a N—H group is replaced to form the covalent bond between PLM and the linker LI.

In one embodiment, the PLM is selected from

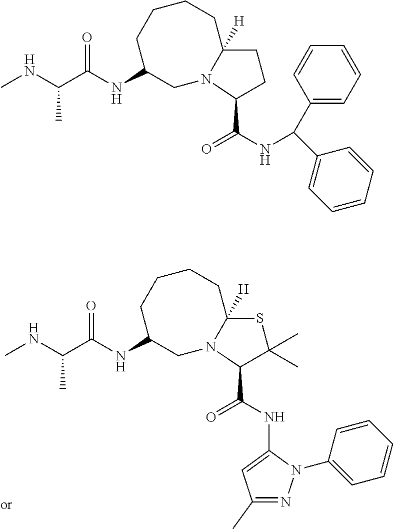

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), is used to form the covalent bond between PLM and the linker LI. In some embodiments, the hydrogen from a N—H group is replaced to form the covalent bond between PLM and the linker LI.

In one embodiment, the PLM is

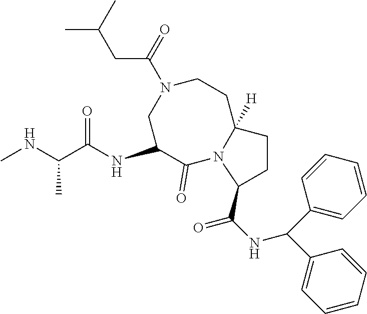

wherein any one of the hydrogen atoms in the PLM can be replaced to form a covalent bond to the LI. In some embodiments, the hydrogen from a N—H group is replaced to form the covalent bond between PLM and the linker LI.

In one embodiment, the PLM is selected from

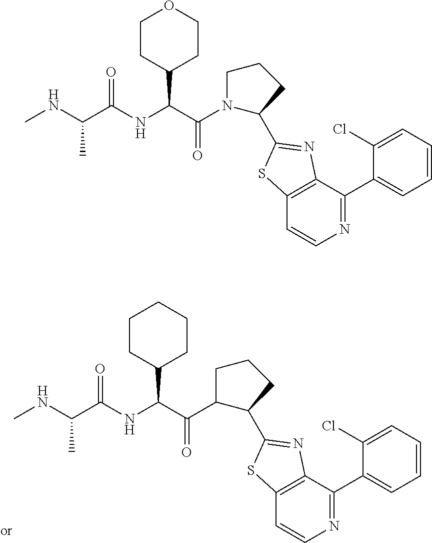

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

In one embodiment, the PLM is selected from

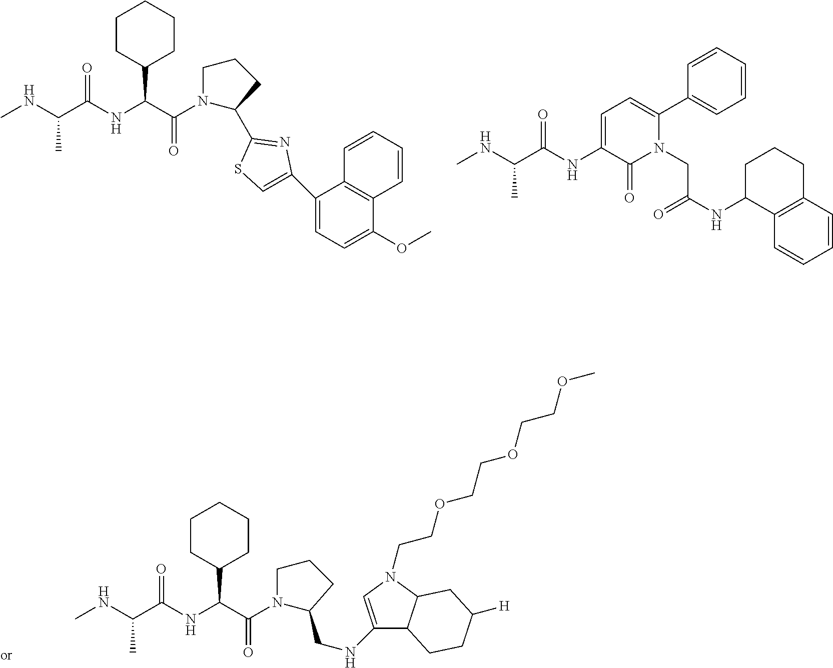

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

In one embodiment, the PLM is selected from

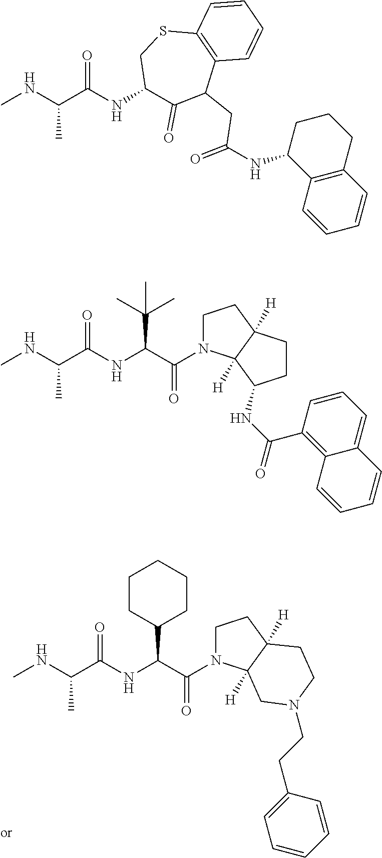 [SMILES: CN[C@@H](C)C(=O)N[C@@H]1CSc2ccccc2C(CC(=O)N[C@@H]2CCCc3ccccc32)C1=O ; CN[C@@H](C)C(=O)N[C@H](C(=O)N1CC[C@H]2CC[C@H](NC(=O)c3cccc4ccccc34)[C@H]21)C(C)(C)C ; CN[C@@H](C)C(=O)N[C@H](C(=O)N1CC[C@H]2CCN(CCc3ccccc3)C[C@H]21)C1CCCCC1]

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

In one embodiment, the PLM is selected from

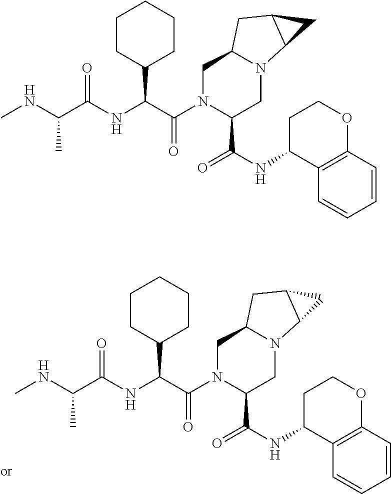 [SMILES: CN[C@@H](C)C(=O)N[C@H](C(=O)N1C[C@H]2C[C@H]3C[C@H]3N2C[C@H]1C(=O)N[C@@H]1CCOc2ccccc21)C1CCCCC1 ; CN[C@@H](C)C(=O)N[C@H](C(=O)N1C[C@H]2C[C@@H]3C[C@@H]3N2C[C@H]1C(=O)N[C@@H]1CCOc2ccccc21)C1CCCCC1]

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

In one embodiment, the PLM is selected from

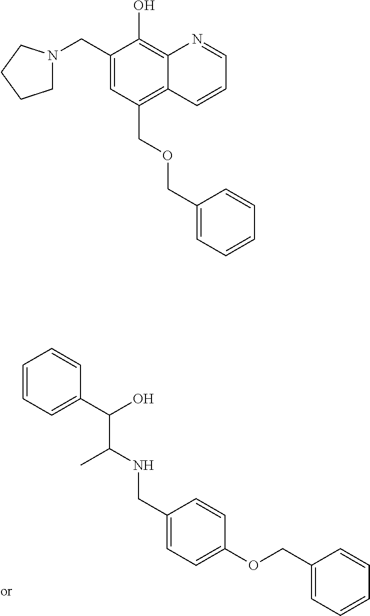 [SMILES: Oc1c(CN2CCCC2)cc(COCc2ccccc2)c2cccnc12 ; CC(NCc1ccc(OCc2ccccc2)cc1)C(O)c1ccccc1]

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

In one embodiment, the PLM is selected from

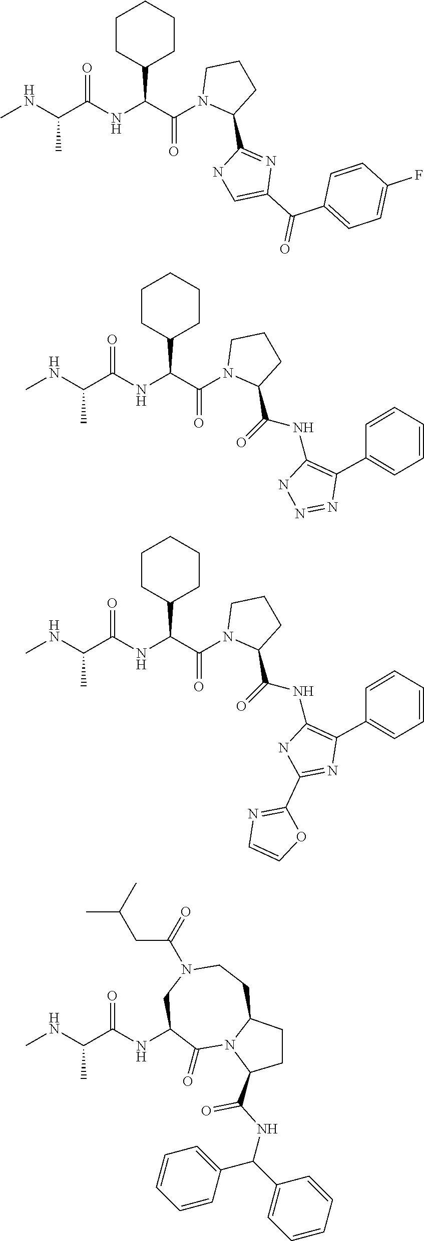 [SMILES: CN[C@@H](C)C(=O)N[C@H](C(=O)N1CCC[C@H]1c1nc(C(=O)c2ccc(F)cc2)c[nH]1)C1CCCCC1 ; CN[C@@H](C)C(=O)N[C@H](C(=O)N1CCC[C@H]1C(=O)Nc1[nH]nnc1-c1ccccc1)C1CCCCC1 ; CN[C@@H](C)C(=O)N[C@H](C(=O)N1CCC[C@H]1C(=O)Nc1[nH]c(-c2ncco2)nc1-c1ccccc1)C1CCCCC1 ; CN[C@@H](C)C(=O)N[C@H]1CN(C(=O)CC(C)C)CC[C@H]2CC[C@@H](C(=O)NC(c3ccccc3)c3ccccc3)N2C1=O]

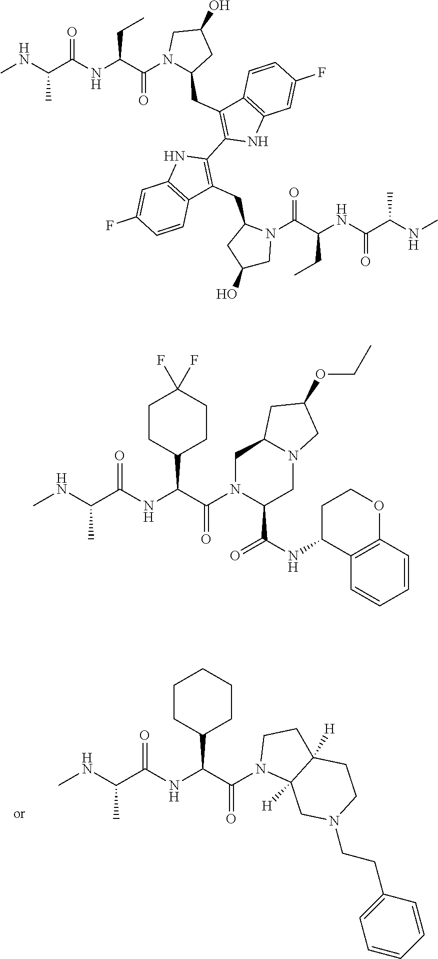

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

In one embodiment, the PLM is selected from

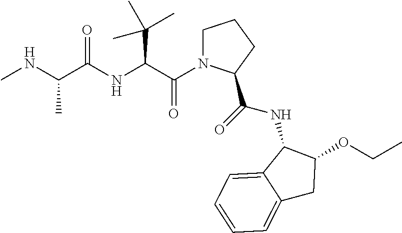 [SMILES: CCO[C@@H]1Cc2ccccc2[C@@H]1NC(=O)[C@@H]1CCCN1C(=O)[C@@H](NC(=O)[C@H](C)NC)C(C)(C)C]

wherein one atom or one chemical group in the PLM is replaced to form a covalent bond to the LI. In some embodiments, one atom in the PLM is replaced to form a covalent bond to the LI. In some embodiments, one chemical group in the PLM is replaced to form a covalent bond to the LI. In some embodiments, a hydrogen (e.g., C—H, N—H), or halogen is used to form the covalent bond between PLM and the linker LI. In some embodiments, the hydrogen from a N—H group is replaced to form the covalent bond between PLM and the linker LI.

### Therapeutic Use

The present compounds find use in any number of methods. For example, in some embodiments the compounds are useful in methods for modulating androgen receptor (AR). Accordingly, in one embodiment, the present disclosure provides the use of compounds of formula (Q) wherein the PTC has the structure of formula (I), (IA), (IB), (IC), (II), (IIA), (IIIA), (IIB), (III), (IV), (IVA), (V), (VA), (VI), (A), (A-I), (B)-(D), (E), (E-I)-(E-VII), (F), (G), (G-I), (G-II), (H), and (H-I) (“formula (I)-(VI) and (A)-(H-I)”), (i)-(iv) or (a), or a pharmaceutically acceptable salt, tautomer, stereoisomer or prodrug thereof, for modulating androgen receptor (AR) activity. For example in some embodiments, modulating androgen receptor (AR) activity is in a mammalian cell. Modulating androgen receptor (AR) can be in a subject in need thereof (e.g., a mammalian subject) and for treatment of any of the described conditions or diseases.

In one embodiment, the modulating AR is binding to AR. In other embodiments, the modulating AR is inhibiting AR.

In one embodiment, the modulating AR is modulating AR N-terminal domain (NTD). In one embodiment, the modulating AR is binding to AR NTD. In other embodiments, the modulating AR is inhibiting AR NTD. In one embodiment, the modulating AR is modulating AR N-terminal domain (NTD). In some embodiments, modulating the AR is inhibiting transactivation of androgen receptor N-terminal domain (NTD).

In other embodiments, modulating androgen receptor (AR) activity is for treatment of at least one indication selected from the group consisting of: prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example in some embodiments, the indication is prostate cancer. In other embodiments, the prostate cancer is primary/localized prostate cancer, locally advanced prostate cancer, recurrent prostate cancer, metastatic prostate cancer, advanced prostate cancer, or metastatic castration-resistant prostate cancer (CRPC), or hormone-sensitive prostate cancer. While in other embodiments, the prostate cancer is androgen dependent prostate cancer. In other embodiments, the spinal and bulbar muscular atrophy is Kennedy's disease.

In one embodiment of the present disclosure, a method of treating a condition associated with cell proliferation in a patient in need thereof is provided, comprising administering a compound of formula (Q) wherein the PTC has the structure of formula (I)-(VI) and (A)-(H-I), (i)-(iv) or (a), or a pharmaceutically acceptable salt, tautomer, stereoisomer or prodrug thereof, to a subject in need thereof. In one embodiment, the present invention provides a method of treating cancer or tumors. In another embodiment, the present invention provides a method of treating prostate cancer or breast cancer.

In another embodiment, the present invention provides a method of treating prostate cancer. In one embodiment, prostate cancer is metastatic castration-resistant prostate cancer.

In another embodiment, the present invention provides a method of treating breast cancer. In one embodiment, breast cancer is triple negative breast cancer.

In one embodiment of the present disclosure, a method of reducing, inhibiting, or ameliorating proliferation, comprising administering a therapeutically effective amount of a compound of formula (Q) wherein the PTC has the structure of formula (I)-(VI) and (A)-(H-I), (i)-(iv) or (a), or a pharmaceutically acceptable salt, tautomer or stereoisomer thereof is provided. In one embodiment, the reducing, inhibiting, or ameliorating in the method disclosed herein, is in vivo. In another embodiment, the reducing, inhibiting, or ameliorating is in vitro.

In one embodiment, the cells in the method disclosed herein, are a cancer cells. In one embodiment, the cancer cells are a prostate cancer cells. In one embodiment, the prostate cancer cells are cells of primary/localized prostate cancer (newly diagnosed or early stage), locally advanced prostate cancer, recurrent prostate cancer (e.g., prostate cancer which was not responsive to primary therapy), metastatic prostate cancer, advanced prostate cancer (e.g., after castration for recurrent prostate cancer), metastatic castration-resistant prostate cancer (CRPC), or hormone-sensitive prostate cancer. In another embodiment, the prostate cancer cells are cells of a metastatic castration-resistant prostate cancer. In other embodiments, the prostate cancer cells are an androgen-dependent prostate cancer cells or an androgen-independent prostate cancer cells. In one embodiment, the cancer cells are breast cancer cells.

In one embodiment, the condition or disease associated with cell proliferation is cancer. In one embodiment of any one of the methods disclosed herein, the cancer is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In one embodiment, the condition or disease is prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In another embodiment, the prostate cancer is a metastatic castration-resistant prostate cancer. In some embodiments, the prostate cancer is an androgen-dependent prostate cancer cells or an androgen-independent prostate cancer. In one embodiment, the condition or disease is breast cancer.

In another embodiment of the present disclosure, a method for reducing or preventing tumor growth, comprising contacting tumor cells with a therapeutically effective amount of a compound of formula (Q) wherein the PTC has the structure of formula (I)-(VI) and (A)-(H-I), (i)-(iv) or (a), or a pharmaceutically acceptable salt, tautomer or stereoisomer thereof is provided.

In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth. In one embodiment, reducing or preventing tumor growth includes reduction in the rate of tumor growth such that the rate of tumor growth before treating a patient with the methods disclosed herein (r1) is faster than the rate of tumor growth after said treatment (r2) such that r1>r2.

In one embodiment, the reducing or preventing in the method disclosed herein is in vivo. In another embodiment, the treating is in vitro.

In one embodiment, the tumor cell in the method disclosed herein is selected from prostate cancer, breast cancer, ovarian cancer, endometrial cancer, or salivary gland carcinoma. In one embodiment, the tumor cells are prostate cancer tumor cells. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the tumor cells are is breast cancer tumor cells.

### Pharmaceutical Compositions and Formulations

The present disclosure also includes pharmaceutical compositions for modulating androgen receptor (AR) in a subject. In one embodiment, a pharmaceutical composition comprises one or more compounds of formula (Q) wherein the PTC has the structure of formula (I), (IA), (IB), (IC), (II), (IIA), (IIIA), (IIB), (III), (IV), (IVA), (V), (VA), (VI), (A), (A-I), (B)-(D), (E), (E-I)-(E-VII), (F), (G), (G-I), (G-II), (H), and (H-I) (“formula (I)-(VI) and (A)-(H-I)”), (i)-(iv) or (a), or a pharmaceutically acceptable salt or solvate thereof.

In one embodiment of the present disclosure, a pharmaceutical composition comprises a therapeutically effective amounts of one or more compounds of formula (Q) wherein the PTC has the structure of formula (I)-(VI) and (A)-(H-I), (i)-(iv) or (a), or a pharmaceutically acceptable salt or solvate thereof.

In a specific embodiment, a pharmaceutical composition, as described herein, comprises one or more compounds of formula (Q) wherein the PTC is selected from Table A, or a pharmaceutically acceptable salt or solvate thereof. In one embodiment, a pharmaceutical composition as described herein comprise one or more compounds of formula (Q) wherein the PTC has the structure selected from Table B, or a pharmaceutically acceptable salt or solvate thereof.

In a specific embodiment, a pharmaceutical composition, as described herein, comprises one or more compounds of formula (Q) wherein the PTC has the structure selected from A1-A234 or B1-B11, or a pharmaceutically acceptable salt or solvate thereof.

In one embodiment, a pharmaceutical composition, as described herein, comprising one or more compounds of formula (Q) wherein the PTC has the structure of formula (I)-(VI) and (A)-(H-I), (i)-(iv) or (a), or a pharmaceutically acceptable salt or solvate thereof, further comprises one or more additional therapeutically active agents. In one embodiment, one or more additional therapeutically active agents are selected from therapeutics useful for treating cancer, neurological disease, a disorder characterized by abnormal accumulation of α-synuclein, a disorder of an aging process, cardiovascular disease, bacterial infection, viral infection, mitochondrial related disease, mental retardation, deafness, blindness, diabetes, obesity, autoimmune disease, glaucoma, Leber's Hereditary Optic Neuropathy, and rheumatoid arthritis.

In some embodiments, the one or more additional therapeutic agents is a a poly (ADP-ribose) polymerase (PARP) inhibitor including but not limited to olaparib, niraparib, rucaparib, talazoparib; an androgen receptor ligand binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide, a 5-alpha reductase inhibitor including but not limited to finasteride, dutasteride, turosteride, bexlosteride, izonsteride, FCE 28260, SKF105,111; a vascular endothelial growth factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an N-terminal domain inhibitor including but not limited to a sintokamide; an alpha-particle emitting radioactive therapeutic agent including but not limited to radium 233 or a salt thereof; niclosamide; or related compounds thereof; a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromitase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; CDK4/6 inhibitors including palbociclib, abemaciclib, ribociclib; HER2 receptor antagonist including but not limited to trastuzumab, neratinib; a mammalian target of rapamycin (mTOR) inhibitor including but not limited to everolimus, temsirolimus.

In a further embodiment of the present disclosure, a pharmaceutical composition comprising one or more compounds of formula (Q) wherein the PTC has the structure of formula I)-(VI) and (A)-(H-I), (i)-(iv) or (a), or a pharmaceutically acceptable salt or solvate thereof, and a pharmaceutically acceptable excipient or adjuvant is provided. The pharmaceutically acceptable excipients and adjuvants are added to the composition or formulation for a variety of purposes. In another embodiment, a pharmaceutical composition comprising one or more compounds of formula (Q) wherein the PTC has the structure of formula (I)-(VI) and (A)-(H-I), (i)-(iv) or (a), or a pharmaceutically acceptable salt or solvate thereof, further comprises a pharmaceutically acceptable carrier. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, the present disclosure provides a pharmaceutical composition comprising a compound of formula (Q) wherein the PTC has the structure of formula (I)-(VI) and (A)-(H-I), (i)-(iv) or (a), or a pharmaceutically acceptable salt or solvate thereof, as disclosed herein, combined with a pharmaceutically acceptable carrier. In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared using compounds of formula (Q) wherein the PTC has the structure of formula (I)-(VI) and (A)-(H-I), (i)-(iv) or (a), or a pharmaceutically acceptable salt or solvate thereof, and any other solid excipients where the components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The agents or compounds, or pharmaceutically acceptable salts or derivatives thereof, are administered orally, nasally, transdermally, pulmonary, inhalationally, buccally, sublingually, intraperintoneally, subcutaneously, intramuscularly, intravenously, rectally, intrapleurally, intrathecally, intraportally, and parenterally. Alternatively or in addition, the route of administration is local, e.g., topical, intra-tumor and peri-tumor. In some embodiments, the compound is administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments the compound of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more compounds of formula (Q) wherein the PTC has the structure of formula (I), (IA), (IB), (IC), (II), (IIA), (IIIA), (IIB), (III), (IV), (IVA), (V), (VA), (VI), (A), (A-I), (B)-(D), (E), (E-I)-(E-VII), (F), (G), (G-I), (G-II), (H), and (H-I) (“formula (I)-(VI) and (A)-(H-I)”), or (a), or a pharmaceutically acceptable salt or solvate thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In various aspects, the amount of the PTCs of formula (Q) wherein the PTC has the structure of formula (I)-(VI) and (A)-(H-I), (i)-(iv), or (a), or a pharmaceutically acceptable salt or solvate thereof, or compounds disclosed in Tables A and B, or a pharmaceutically acceptable salt or solvate thereof, can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention, and are not intended to limit the invention.

Synthetic Preparation

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

Compounds of the present invention can be prepared by the literature methods cited in the following text. The following schemes depict established, known syntheses of these scaffolds.

The groups and/or the substituents of the compounds of the present invention can be synthesized and attached to these scaffolds by the literature methods cited in the following text. The following schemes depict the known techniques for accomplishing this joinder.

### GENERAL SYNTHESIS

Compounds of the present invention can be synthesized using the following methods. General reaction conditions are given, and reaction products can be purified by general known methods including crystallization, silica gel chromatography using various organic solvents such as hexane, cyclohexane, ethyl acetate, methanol and the like, preparative high pressure liquid chromatography or preparative reverse phase high pressure liquid chromatography.

Representative Synthesis of PTCs

For synthesis of Compounds in Tables A and B, see PCT/US2019/057034 for procedures. The disclosures of PCT/US2019/057034 are hereby incorporated by reference in their entireties.

### Example 1: Synthesis of 5-[[4-[1-[3,5-dichloro-4-(3-chloropropoxy)phenyl]-1-methyl-ethyl]phenoxy]methyl]-4-methylsulfonyl-oxazole (A3)

To a suspension of 4-[1-[3,5-dichloro-4-(3-chloropropoxy)phenyl]-1-methyl-ethyl]phenol (7) (0.135 g, 0.36 mmol) and Cs₂CO₃ (0.197 g, 0.6 mmol) in DMF (3 mL) was added (4-methylsulfonyloxazol-5-yl)methyl 4-methylbenzenesulfonate (2) (0.1 g, 0.3 mmol) at 25° C. The mixture was stirred at 60° C. for 6 hours. LCMS showed the reaction was completed. The resulting mixture was poured into H₂O (8 mL) and extracted with EtOAc (5 mL×3). The combined organic layers were washed with brine (5 mL×2), dried over Na₂SO₄, filtered, and concentrated under reduced pressure. The residue was purified by prep-HPLC (HCl) to give 5-[[4-[1-[3,5-dichloro-4-(3-chloropropoxy)phenyl]-1-methyl-ethyl]phenoxy]methyl]-4-methylsulfonyl-oxazole (A1) (37.9 mg, yield: 23.6%) as yellow oil. HPLC purity (220 nm): 96.25%. ¹H NMR (400 MHz, CHCl₃-d) δ 7.99 (s, 1H), 7.16-7.10 (m, 4H), 6.94 (d, J=8.82 Hz, 2H), 5.42 (s, 2H), 4.15 (t, J=5.73 Hz, 2H), 3.86 (t, J=6.50 Hz, 2H) 3.18 (s, 3H), 2.28 (quin, J=6.17 Hz, 2H), 1.62 (s, 6H). LCMS (M+23) m/z: calcd 533; found 556.

### Example 2: Synthesis of 4-((4-(2-(3,5-dichloro-4-(3-chloropropoxy)phenyl)propan-2-yl)phenoxy) methyl)-1-(methylsulfonyl)-1H-imidazole (A5)

To a mixture of 4-((4-(2-(3,5-dichloro-4-(3-chloropro poxy)phenyl)propan-2-yl)phenoxy)methyl)-1H-imidazole (6) (80 mg, 0.2 mmol) and TEA (0.1 mL, 0.5 mmol) in DCM (2 mL) was added methanesulfonyl chloride (41 mg, 0.4 mmol) dropwise at 0° C., and the mixture was stirred at 25° C. for 2 hours. TLC showed the reaction was completed. The mixture was diluted with water (20 mL), extracted with DCM (5 mL×3), and the combined organic layers were washed with brine (10 mL), dried over Na₂SO₄, filtered and concentrated under reduced pressure. The residue was purified by prep-HPLC (NH₄HCO₃) to give 4-((4-(2-(3,5-dichloro-4-(3-chloropropoxy)phenyl)propan-2-yl)phenoxy)methyl)-1-(methyl sulfonyl)-imidazole (A5) (16 mg, yield: 16.6%) as colorless oil. ¹H NMR (400 MHz, CHCl₃-d) δ=7.99 (d, J=1.3 Hz, 1H), 7.40 (s, 1H), 7.15-7.12 (m, 4H), 6.95-6.90 (m, 2H), 5.05 (s, 2H), 4.15 (t, J=5.7 Hz, 2H), 3.86 (t, J=6.4 Hz, 2H), 3.30 (s, 3H), 2.31-2.26 (m, 2H), 1.63 (s, 6H). LCMS (220 nm): 95.2%. LCMS (M+1) m/z: calcd 530.1; found 531.0.

### Example 3: Synthesis of 2-((4-(2-(3,5-dichloro-4-(3-chloropropoxy)phenyl)propan-2-yl)phenoxy)methyl)-5-(methylsulfonyl)-1, 3, 4-oxadiazole (A7)

To a solution of 3-(1-(4-(2-(3,5-dichloro-4-(3-chloropropoxy)phenyl)propan-2-yl)phenoxy)ethyl)-5-(methylthio)-4H-pyrazole (5) (220 mg, 0.49 mmol) in DCM (5 mL) was added m-CPBA (85% purity, 226 mg, 4.03 mmol) at 0° C. The reaction was stirred at 20° C. for 4 hours. LCMS showed the reaction was completed. The mixture was quenched with saturated aqueous Na₂S₂O₃ (5 mL) and saturated aqueous NaHCO₃ (5 mL), then extracted with DCM (10 mL×3). The combined organic layers were washed with brine (10 mL), dried over Na₂SO₄, filtered and concentrated under reduced pressure. The residue was purified by prep-HPLC (TFA) to give 2-((4-(2-(3,5-Dichloro-4-(3-chloropropoxy)phenyl)propan-2-yl)phenoxy)methyl)-5-(methylsulfonyl)-1,3,4-oxadiazole (A7) (74 mg, yield: 31.6%) as colorless oil. ¹H NMR (400 MHz, DMSO-d6) δ ppm 7.14 (d, J=8.9 Hz, 2H), 7.11 (s, 2H), 6.95 (d, J=8.8 Hz, 2H), 5.35 (s, 2H), 4.15 (t, J=5.73 Hz, 2H), 3.86 (t, J=6.39 Hz, 2H), 3.50 (s, 3H), 2.28 (t, J=6.06 Hz, 2H), 1.63 (s, 6H). LCMS (220 nm): 97%. LCMS M+H⁺) m/z: calcd 532.04, found 533.1.

### Example 4: Synthesis of 5-((4-(2-(3,5-dichloro-4-(2-chloroethoxy)phenyl)propan-2-yl)phenoxy)methyl)-4-(methylsulfonyl)oxazole (A13)

A solution of 5-(chloromethyl)-4-methylsulfonyl-oxazole (6) (500 mg, 2.56 mmol), 4-[1-[3,5-dichloro-4-(2-chloroethoxy)phenyl]-1-methyl-ethyl]phenol (11) (919 mg, 2.56 mmol) and Cs₂CO₃ (1.67 g, 5.11 mmol) in DMF (20 mL) was stirred at 25° C. for 2 hours. Then the resulting solution was stirred at 40° C. for 0.5 hr. The reaction was completed detected by TLC. The reaction was quenched with water (50 mL), and the mixture was extracted with EtOAc (20 mL×3). The combined organic layers were washed with brine (30 mL×2), dried over Na₂SO₄, filtered, and concentrated under reduced pressure. The residue was purified by MPLC to give 5-[[4-[1-[3,5-dichloro-4-(2-chloroethoxy)phenyl]-1-methyl-ethyl]phenoxy]methyl]-4-methylsulfonyl-oxazole (528 mg, yield: 39.8%) as white solid. ¹H NMR (400 MHz, CHLOROFORM-d) δ=7.92 (s, 1H), 7.09-7.02 (m, 4H), 6.89-6.83 (m, 2H), 5.34 (s, 2H), 4.18 (t, J=6.4 Hz, 2H), 3.79 (t, J=6.4 Hz, 2H), 3.11 (s, 3H), 1.54 (s, 6H). MS(M+H⁺) m/z: clcd. 517.0; found 518.1, 540.0.

### Example 5: Synthesis of N-((3-(4-(2-(3,5-dichloro-4-(3-chloropropoxy)phenyl)propan-2-yl)phenyl) isoxazol-5-yl)methyl)methanesulfonamide (A22)

To a solution of [3-[4-[1-[3,5-dichloro-4-(3-chloropropoxy)phenyl]-1-methyl-ethyl]phenyl]isoxazol-5-yl]methanamine (7) (60 mg, 0.13 mmol) in DCM (3 mL) was added TEA (40 mg, 0.40 mmol) and MsCl (18 mg, 0.16 mmol) under N2 atmosphere at 0° C. The reaction was stirred at 20° C. for 5 hrs. TLC showed the reaction was completed. The mixture was poured into H₂O (5 mL) and extracted with DCM (10 mL×3). The combined organic layers were washed with brine (10 mL), dried over Na₂SO₄, filtered and concentrated under reduced pressure. The residue was purified by prep-HPLC (TFA) to give N-((3-(4-(2-(3,5-dichloro-4-(3-chloropropoxy)phenyl)propan-2-yl)phenyl)isoxazol-5-yl)methyl) methanesulfonamide (A22) (5 mg, yield: 7.11%) as brown oil. LCMS purity (220 nm): 89.4%. ¹H NMR (400 MHz, CHCl₃-d) δ=7.73 (br d, J=7.9 Hz, 2H), 7.31 (br d, J=8.2 Hz, 2H), 7.14 (s, 2H), 6.60 (s, 1H), 4.89-4.80 (m, 1H), 4.54 (d, J=6.2 Hz, 2H), 4.16 (t, J=5.6 Hz, 2H), 3.86 (t, J=6.4 Hz, 2H), 2.99 (s, 3H), 2.33-2.25 (m, 2H), 1.68 (s, 6H). LCMS(M+H⁺) m/z: clcd. 530.0; found 531.0.

### Example 6: Synthesis of N-(tert-Butyl)-3,5-dichloro-4-(2-chloroethoxy)-N-(4-((4-(methyl-sulfonyl)oxazol-5-yl)methoxy)phenyl)aniline (A31)

To a mixture of 4-[N-tert-butyl-3,5-dichloro-4-(2-chloroethoxy)anilino]phenol (9) (110 mg, 0.283 mmol) and Cs₂CO₃ (277 mg, 0.85 mmol) in DMF (5 mL) was added 5-(chloromethyl)-4-methylsulfonyl-oxazole (10) (83 mg, 0.42 mmol). Then the resulting mixture was stirred at 40° C. for 2 hours. LCMS showed the reaction was completed. The mixture was cooled down, quenched with water (5 mL) and extracted with EtAOc (5 mL×3). The combined organic layers were washed with brine (5 mL×3), dried over Na₂SO₄, filtered and concentrated under reduced pressure. The residue was purified by p-HPLC (TFA) to give the N-tert-butyl-3,5-dichloro-4-(2-chloroethoxy)-N-[4-[(4-methylsulfonyl-oxazol-5-yl)-methoxy]-phenyl]aniline (A31) (36.5 mg, yield: 23.5%) as yellow solid. HPLC purity (220 nm): 91.7%. ¹H NMR (400 MHz, CHCl₃-d) δ 8.01 (s, 1H), 7.06-7.02 (m, 2H), 6.99-6.94 (m, 2H), 6.73 (s, 2H), 5.41 (s, 2H), 4.17 (t, J=6.4 Hz, 2H), 3.82 (t, J=6.4 Hz, 2H), 3.20 (s, 3H), 1.35 (s, 9H). LCMS (M+Na⁺) m/z: calcd 546.1; found 569.1.

### Example 7: Synthesis of 4-(2-(3,5-dichloro-4-(2-chloroethoxy)phenyl)propan-2-yl)-N-((4-(methylsulfonyl)oxazol-5-yl)methyl)aniline hydrochloride (A32)

To a suspension of 5-(chloromethyl)-4-(methylsulfonyl)oxazole (5) (200 mg, 0.5 mmol) and Ag₂CO₃ (564 mg, 0.2 mmol) in DMF (2 mL) was added 4-(2-(3,5-dichloro-4-(2-chloroethoxy)phenyl)propan-2-yl)aniline (4) (382 mg, 0.1 mmol), and the mixture was stirred at 65° C. for 2 hours. TLC showed the reaction was completed. The resulting mixture was cooled down, poured into H₂O (6 mL), extracted with EtOAc (2 mL×2). The combined organic layers were washed with brine (4 mL×3), dried over Na₂SO₄, filtered and concentrated under reduced pressure. The residue was purified by prep-HPLC (HCl) to give 4-(2-(3,5-dichloro-4-(2-chloroethoxy)phenyl)propan-2-yl)-N-((4-(methylsulfonyl)oxazol-5-yl) methyl)aniline hydrochloride (A32) (20 mg, yield: 3.8%) as white solid. ¹H NMR (400 MHz, CHCl₃-d) δ 7.85 (s, 1H), 7.16-7.09 (m, 4H), 7.04-6.93 (m, 2H), 4.80 (s, 2H), 4.26 (t, J=6.4 Hz, 2H), 3.86 (t, J=6.4 Hz, 2H), 3.16 (s, 3H), 1.61 (s, 6H). LCMS (M+H⁺) m/z: calcd: 516.0; found 517.0.

### Example 8: Synthesis of 5-(1-(4-(2-(3,5-dichloro-4-(2-chloroethoxy)phenyl)propan-2-yl) phenoxy)ethyl)-4-(methylsulfonyl)oxazole (A35)

To a mixture of 5-(1-(4-(2-(3,5-dichloro-4-(2-chloroethoxy)phenyl)propan-2-yl)phenoxy)ethyl)-4-(methylthio)oxazole (8) (50 mg, 0.1 mmol) was added mCPBA (80% purity, 64 mg, 0.3 mmol) in DCM (3 mL) at 25° C., and the mixture was stirred at the same temperature for 16 hours. LCMS showed the reaction was completed. The reaction was quenched with H₂O (5 mL), extracted with EtOAc (6 mL×3). The combined organic layers were washed with brine (3 mL), dried over Na₂SO₄, filtered and concentrated under reduced pressure. The residue was purified by p-HPLC (TFA) 5-(1-(4-(2-(3,5-dichloro-4-(2-chloroethoxy)phenyl)propan-2-yl)phenoxy)ethyl)-4-(methylsulfonyl)oxazole (21.7 mg, yield: 40.8%) as white solid. HPLC purity (220 nm): 98.5%. ¹H NMR (400 MHz, CHCl₃-d) δ=7.94 (s, 1H), 7.14-7.03 (m, 4H), 6.92 (d, J=8.9 Hz, 2H), 6.10 (q, J=6.5 Hz, 1H), 4.26 (t, J=6.3 Hz, 2H), 3.86 (t, J=6.3 Hz, 2H), 3.06 (s, 3H), 1.74 (d, J=6.7 Hz, 3H), 1.59 (s, 6H). LCMS (M+H⁺) m/z: calcd: 531.0; found 532.0.

### Example 9: Synthesis of N-(4-((4-(2-(3,5-dichloro-4-(2-chloroethoxy)phenyl)propan-2-yl)phenoxy)methyl)oxazol-2-yl)methanesulfonamide (A38)

To a solution of 2-chloro-4-[[4-[1-[3,5-dichloro-4-(2-chloroethoxy)phenyl]-1-methyl-ethyl]phenoxy]methyl]oxazole (5) (10 mg, 0.02 mmol) in 1,4-dioxane (0.2 mL) was added methanesulfonamide (2.4 mg, 0.02 mmol), Brettphos Pd G3 (2 mg, w20%) and t-BuONa (3 mg, 0.03 mmol). The mixture was stirred at 80° C. for 10 hours under N2 atmosphere. LCMS showed 5% desired MS and 90% starting material. The resulting 20 reaction mixtures were cooled down and combined. The mixture was filtered and the filtrate concentrated under reduced pressure. The residue was purified by prep-HPLC (TFA) to give N-[[5-bromo-4-[4-[1-[3,5-dichloro-4-(3-chloropropoxy)phenyl]-1-methyl-ethyl]phenyl]oxazol-2-yl]methyl]methanesulfonamide (2 mg, yield: 1.8%) as pale yellow solid. LCMS (220 nm): 85.79%. ¹H NMR (400 MHz, CHCl₃-d) δ 7.16 (d, J=8.8 Hz, 2H), 7.12 (s, 2H), 7.08 (s, 1H), 6.86 (d, J=8.8 Hz, 2H), 4.86 (s, 2H), 4.27 (t, J=6.4 Hz, 2H), 3.86 (t, J=6.4 Hz, 2H), 3.09 (s, 3H), 1.64 (s, 6H). LCMS (M+H⁺) m/z: calcd: 532.04; found 533.0.

### Example 10: Synthesis of N-[3-[[4-[1-[3,5-dichloro-4-(2-chloroethoxy)phenyl]-1-methyl-ethyl]phenoxy] methyl]-1H-pyrazol-4-yl]methanesulfonamide (A40)

A solution of N-[3-[[4-[1-[3,5-dichloro-4-(2-chloroethoxy)phenyl]-1-methyl-ethyl]phenoxy]methyl]-1-tetrahydropyran-2-yl-pyrazol-4-yl]methanesulfonamide (9) (70 mg, 0.113 mmol) in HCl/EtOAc (4M, 2 mL) was stirred at 20° C. for 2 hours. LCMS showed the reaction was completed. The mixture was concentrated under reduced pressure. The residue was purified by prep-HPLC (FA) to give N-[3-[[4-[1-[3,5-dichloro-4-(2-chloroethoxy)phenyl]-1-methyl-ethyl]phenoxy]methyl]-1H-pyrazol-4-yl]methanesulfonamide(A40) (11.6 mg, yield: 18.2%) as white solid. ¹H NMR (400 MHz, CHCl₃-d) δ p pm 7.71 (s, 1H), 7.10-7.17 (m, 4H), 6.89-6.94 (m, 2H), 6.22 (s, 1H), 5.21 (s, 2H), 4.26 (t, J=6.39 Hz, 2H), 3.86 (t, J=6.28 Hz, 2H), 2.90 (s, 3H), 1.62 (s, 6H). LCMS (M+Na⁺) m/z: calcd: 531.06; found 532.1.

### Example 11: Synthesis of N-(4-((4-(2-(3, 5-dichloro-4-(2-chloroethoxy)phenyl)propan-2-yl)phenoxy) methyl) pyrimidin-5-yl)methanesulfonamide (A41)

A mixture of tert-butyl N-(4-((4-(1-(3,5-dichloror-4-(2-chloroethoxy)phenyl)-1-methyl-ethyl)phenoxy)methyl)pyrimidin-5-yl)-N-methylsulfonyl-carbamate (6) (50 mg, 0.062 mmol) in DCM (5.0 mL) and TFA (0.5 mL) was stirred at 20° C. for 1 hour. LCMS showed the reaction was completed. The resulting mixture was concentrated under reduced pressure. The residue was purified by prep-HPLC (TFA) to give N-(4-((4-(1-(3,5-dichloro-4-(2-chloroethoxy)phenyl)-1-methyl-ethyl)phenoxy)methyl)pyrimidin-5-yl) methanesulfonamide (A41) (8 mg, yield: 23.7%) as yellow oil. ¹H NMR (400 MHz, CHCl3-d) δ ppm 9.01 (d, J=4.40 Hz, 2H), 7.81 (br s, 1H), 7.16 (d, J=8.93 Hz, 2H), 7.10 (s, 2H), 6.93 (d, J=8.93 Hz, 2H), 5.36 (s, 2H), 4.26 (t, J=6.36 Hz, 2H), 3.86 (t, J=6.36 Hz, 2H), 3.03 (s, 3H), 1.62 (s, 6H). LCMS (M+H⁺) m/z: 545.05; found 546.0. HPLC purity(220 nm): 84.4%.

### Example 12: Synthesis of N-(4-(2-(3,5-dichloro-4-(2-chloroethoxy)phenyl)propan-2-yl)phenyl)-2-(methylsulfonamido)oxazole-4-carboxamide (A49)

To a solution of 2-(methane-sulfonamido)oxazole-4-carboxylic acid (3) (60 mg, 0.3 mmol) in DMF (3 mL) was added 4-[1-[3,5-dichloro-4-(2-chloroethoxy)phenyl]-1-methyl-ethyl]aniline (4) (104 mg, 0.3 mmol), HATU (133 mg, 0.35 mmol) and TEA (0.12 mL, 0.9 mmol) at 25° C. The mixture was stirred at the same temperature for 3 hours. LCMS showed the reaction was completed, the mixture was quenched with H₂O (1 mL), and directly purified by prep-HPLC (TFA), to give N-[4-[1-[3,5-dichloro-4-(2-chloroethoxy)phenyl]-1-methyl-ethyl]phenyl]-2-(methanesulfonamido)oxazole-4-carboxamide (A49) (23.2 mg, yield: 14.6%) as white solid. ¹H NMR (400 MHz, CHCl₃-d) δ 8.44 (s, 1H), 7.91 (s, 1H), 7.56 (d, J=8.8 Hz, 2H), 7.21 (d, J=8.8 Hz, 2H), 7.13 (s, 2H), 4.27 (t, J=6.4 Hz, 2H), 3.86 (t, J=6.4 Hz, 2H), 3.32 (s, 3H), 1.65 (s, 6H). LCMS (M+H⁺) m/z: clcd 545.03; found 546.0.

### Example 13: Synthesis of 5-((4-(2-(3,5-dichloro-4-(3,3,3-trifluoropropoxy)phenyl)propan-2-yl)phenoxy)methyl)-4-(methylsulfonyl)oxazole (A54)

To a mixture of 4-[1-[3,5-dichloro-4-(3,3,3-trifluoro propoxy)phenyl]-1-methyl-ethyl]phenol (3) (40 mg, 0.10 mmol) and 5-(chloromethyl)-4-methylsulfonyl-oxazole (4) (24 mg, 0.12 mmol) in DMF (0.5 mL) was added Cs₂CO₃ (66 mg, 0.20 mmol) and the mixture was stirred at 25° C. for 16 hours. LCMS showed the reaction was completed. The mixture was poured into water (2 mL) and extracted with EtOAc (5 mL×3). The combined organic layers were washed with brine (5 mL×3), dried over Na₂SO₄, filtered and concentrated under reduced pressure. The residue was purified by p-TLC to give 5-[[4-[1-[3,5-dichloro-4-(3,3,3-trifluoropropoxy)phenyl]-1-methyl-ethyl]phenoxy]methyl]-4-methylsulfonylo-xazole (A54) (18 mg, yield: 29.9%) as yellow oil. LCMS purity: (220 nm): 93.3%. ¹H NMR (400 MHz, CHCl₃-d) δ 8.00 (s, 1H), 7.16-7.12 (m, 4H), 6.94 (d, J=8.8 Hz, 2H), 5.42 (s, 2H), 4.22 (t, J=6.8 Hz, 2H), 3.19 (s, 3H), 2.78-2.64 (m, 2H), 1.62 (s, 6H). LCMS (M+NH₄⁺) m/z: calcd 551.1; found 569.0.

### Example 14: Synthesis of 2-(2-chloroethoxy)-5-(2-(3-cyano-4-((4-(methylsulfonyl)oxazol-5-yl)methoxy) phenyl)propan-2-yl)benzonitrile (A63)

To a solution of 2-(2-chloroethoxy)-5-(2-(3-cyano-4-hydroxyphenyl)propan-2-yl)benzonitrile (7) (130 mg, 0.38 mmol) in DMF (2 mL) was added 5-(chloromethyl)-4-(methylsulfonyl)oxazole (G) (75 mg, 0.38 mol) and Cs₂CO₃ (249 mg, 0.76 mmol) under N₂ atmosphere. The reaction was stirred at 0° C. for 3 hours. LCMS showed the reaction was completed. The mixture was diluted with EtOAc (5 mL) and poured into H₂O (5 mL). The aqueous phase was extracted with EtOAc (5 mL×2). The combined organic layers were washed with brine (5 mL×4), then dried over Na₂SO₄, filtered and concentrated under reduced pressure. The crude product was purified by prep-HPLC(TFA) to give 2-(2-chloroethoxy)-5-(2-(3-cyano-4-((4-(methylsulfonyl)oxazol-5-yl)methoxy)phenyl)propan-2-yl)benzonitrile (A63) (53 mg, yield: 27.8%) as white solid. LCMS purity (220 nm): 91.1%. ¹H NMR (400 MHz, CHCl₃-d) δ=8.04 (s, 1H), 7.41 (br s, 2H), 7.39-7.29 (m, 2H), 7.15-7.04 (m, 1H), 6.94-6.85 (d, J=8.9 Hz, 1H), 5.51 (s, 2H), 4.33 (br t, J=6.0 Hz, 2H), 3.87 (br t, J=6.0 Hz, 2H), 3.25 (s, 3H), 1.64 (s, 6H). LCMS (M+H⁺) m/z: calcd 499.1; found 500.1.

### Example 15A: Synthesis of N-((2-((4-(2-(3,5-dichloro-4-(2-chloroethoxy)phenyl)propan-2-yl) phenyl)amino)oxazol-5-yl)methyl)methanesulfonamide (A75)

A solution of tert-butyl N-[[2-[4-[1-[3,5-dichloro-4-(2-chloroethoxy)phenyl]-1-methyl-ethyl]anilino]oxazol-5-yl]methyl]-N-methylsulfonyl-carbamate (5) (25 mg, 0.04 mmol) in DCM (2 mL) and TFA (0.2 mL) was stirred at 25° C. for 3 hours. LCMS showed the reaction was completed. The mixture was concentrated and purified by prep-HPLC (TFA) to give N-[[2-[4-[1-[3,5-dichloro-4-(2-chloroethoxy)phenyl]-1-methyl-ethyl]anilino]oxazol-5-yl]methyl]methanesulfonamide (4.6 mg, yield: 21.9%) as yellow oil. LCMS purity (220 nm): 86%. ¹H NMR (400 MHz, CHCl₃-d) δ 7.36-7.33 (m, 2H), 7.25-7.22 (m, 2H), 7.11 (s, 2H), 7.02 (s, 1H), 5.18 (s, 1H), 4.36 (s, 2H), 4.27 (t, J=6.4 Hz, 2H), 3.87 (t, J=6.4 Hz, 2H), 3.00 (s, 3H), 1.64 (s, 6H). LCMS (M+H⁺) m/z: calcd: 531.0; found 531.6.

### Example 15B: Synthesis of N-(4-((4-(2-(3-chloro-4-(2-chloroethoxy)-5-cyanophenyl)propan-2-yl)phenoxy) methyl)pyrimidin-2-yl)methanesulfonamideN-(4-((4-(2-(3-chloro-4-(2-chloroethoxy)-5-cyanophenyl) propan-2-yl) phenoxy) methyl)pyrimidin-2-yl)methanesulfonamide (A109)

2-chloro-4-(chloromethyl)pyrimidine (2): To a mixture of 2-chloro-4-methyl-pyrimidine (50.0 g, 398 mmol) and NCS (77.9 g, 583 mmol) in MeCN (250 mL) was added benzoyl benzenecarboperoxoate (28.3 g, 117 mmol) in portions at 20° C. and the mixture was stirred at 100° C. for 16 hrs under N2 atmosphere. TLC showed most of the starting material consumed and two new spots appeared. The mixture was cooled down to room temperature, poured into water (500 mL) and extracted with EtOAc (200 mL×3). The organic layers were combined and washed with brine (200 mL×2), dried over Na2SO4, filtered and concentrated under reduced pressure. The residue was purified by silica gel column chromatography to give 2-chloro-4-(chloromethyl) pyrimidine (22 g, yield: 31.2%) as yellow oil. 1H NMR (400 MHz, CDCl3) δ=8.69 (d, J=5.2 Hz, 1H), 7.54 (d, J=5.0 Hz, 1H), 4.61 (s, 2H).

3-chloro-2-(2-chloroethoxy)-5-(2-(4-((2-chloropyrimidin-4-yl)methoxy)phenyl)propan-2-yl)benzonitrile (4): To a mixture of 3-chloro-2-(2-chloroethoxy)-5-(2-(4-hydroxyphenyl)propan-2-yl)benzonitrile (18.0 g, 51.4 mmol) and 2-chloro-4-(chloromethyl) pyrimidine (10.1 g, 61.7 mmol) in DMF (150 mL) was added Cs2CO3 (33.5 g, 103.4 mmol) at 20° C. and the mixture was stirred at the same temperature for 16 hrs. LCMS showed the reaction was completed. The reaction mixture was poured into H₂O (300 mL) and extracted with EtOAc (150 mL×3). The combined organic layers were washed with brine (150 mL×3), dried over Na₂SO₄, filtered and concentrated under reduced pressure. The residue was purified by silica gel column chromatography to give 3-chloro-2-(2-chloroethoxy)-5-(2-(4-((2-chloropyrimidin-4-yl)methoxy)phenyl)propan-2-yl)benzonitrile (15.5 g, yield: 63.3%) as white solid. 1H NMR (400 MHz, CDCl3) δ=8.67 (d, J=5.2 Hz, 1H), 7.56 (d, J=5.2 Hz, 1H), 7.45 (d, J=2.4 Hz, 1H), 7.35-7.29 (m, 1H), 7.13 (d, J=8.8 Hz, 2H), 6.90 (d, J=8.8 Hz, 2H), 5.16 (s, 2H), 4.43 (t, J=6.0 Hz, 2H), 3.88 (t, J=6.0 Hz, 2H), 1.65 (s, 6H).

N-(4-((4-(2-(3-chloro-4-(2-chloroethoxy)-5-cyanophenyl)propan-2-yl)phenoxy)methyl) pyrimidin-2-yl)methanesulfonamide (A109): To a mixture of 3-chloro-2-(2-chloroethoxy)-5-(2-(4-((2-chloropyrimidin-4-yl)methoxy)phenyl)propan-2-yl)benzonitrile (15.5 g, 32.5 mmol), methane sulfonamide (9.3 g, 97.5 mmol), Cs2CO3 (21.2 g, 65.0 mmol) and Xantphos (1.88 g, 3.25 mmol) in 1,4-dioxane (450 mL) was added Pd2(dba)3 (3.0 g, 3.3 mmol) at 20° C. and the mixture was stirred at 90° C. for 6 hrs under N2 atmosphere. LCMS showed the reaction was completed. The mixture was cooled down to room temperature, poured into water (300 mL) and extracted with EtOAc (300 mL×3). The combined organic layers were washed with brine (300 mL×2), dried over Na2SO4, filtered and concentrated under reduced pressure. The residue was purified by silica gel column chromatography to give the crude product and then further purified by p-HPLC (TFA) to give N-(4-((4-(2-(3-chloro-4-(2-chloroethoxy)-5-cyanophenyl)propan-2-yl)phenoxy) methyl)pyrimidin-2-yl)methanesulfonamide (5.30 g, yield: 30.1%) as yellow solid. 1H NMR (400 MHz, CDCl3) δ=10.02 (br s, 1H), 8.69 (d, J=5.2 Hz, 1H), 7.45 (d, J=2.4 Hz, 1H), 7.34-7.31 (m, 1H), 7.30 (d, J=5.2 Hz, 1H), 7.13 (d, J=8.8 Hz, 2H), 6.91 (d, J=8.8 Hz, 2H), 5.13 (s, 2H), 4.43 (t, J=6.0 Hz, 2H), 3.88 (t, J=6.0 Hz, 2H), 3.47 (s, 3H), 1.65 (s, 6H). LCMS (220 nm): 99.0%. Exact Mass: 534.09; found 535.1, 537.0. See PCT/US2019/057034.

### Example 16: Synthesis of 3-(4-(2-(3,5-dichloro-4-(2-chloroethoxy)phenyl)propan-2-yl)benzyl)-1,5,5-trimethylimidazolidine-2,4-dione (B2)

To a mixture of 1,5,5-trimethylimidazolidine-2,4-dione (5) (20 mg, 0.2 mmol) and K₂CO₃ (70 mg, 0.5 mmol) in DMF (3 mL) was added 1,3-dichloro-2-(2-chloroethoxy)-5-(2-(4-(chloromethyl)phenyl)propan-2-yl)benzene (4) (50 mg, 0.1 mmol) at 25° C. and the mixture was stirred at the same temperature for 2 hours. LCMS showed the reaction was completed. The mixture was poured into H₂O (10 mL), extracted with EtOAc (5 mL×2). The combined organic layers were washed with brine (5 mL), dried over Na₂SO₄, filtered and concentrated under reduced pressure. The residue was purified by prep-HPLC (HCl) to give 3-(4-(2-(3,5-dichloro-4-(2-chloroethoxy)phenyl)propan-2-yl)benzyl)-1,5,5-trimethylimidazolidine-2,4-dione (B2) (20 mg, yield: 31.8%) as colorless oil. LCMS purity (220 nm): 96.1%. ¹H NMR (400 MHz, CHCl₃-d) δ=7.30-7.25 (m, 1H), 7.28-7.25 (m, 1H), 7.30-7.25 (m, 1H), 7.15-7.10 (m, 2H), 7.10-7.08 (m, 2H), 4.66-4.57 (m, 2H), 4.24 (t, J=6.4 Hz, 2H), 3.89-3.77 (m, 2H), 2.87 (s, 3H), 1.65-1.54 (m, 6H), 1.41-1.34 (m, 6H). LCMS (M+H⁺) m/z: calcd: 496.1; found 497.1.

### Example 17: Synthesis of 3-(4-(2-(3,5-dichloro-4-(2-chloroethoxy)phenyl)propan-2-yl)benzyl)-5,5-dimethyl-1-(methylsulfonyl)imidazolidine-2,4-dione (B3)

To a solution of 3-(4-(2-(3,5-dichloro-4-(2-chloroethoxy)phenyl)propan-2-yl)benzyl)-5,5-dimethylimidazolidine-2,4-dione (6) ((40 mg, 0.1 mmol) in THF (2 mL) was added Mesyl chloride (0.1 mL, 0.2 mmol) and NaH (60.0%, 6 mg, 0.2 mmol) at 0° C. and the mixture was stirred at 80° C. for 16 hours. TLC showed the reaction was completed. The reaction was quenched with saturated aqueous NH₄Cl (10 mL) and extracted with EtOAc (3 mL×2). The combined organic layers were washed with brine (3 mL), dried over Na₂SO₄, filtered and concentrated under reduced pressure. The residue was purified by prep-HPLC (HCl) to give 3-(4-(2-(3,5-dichloro-4-(2-chloroethoxy)phenyl)propan-2-yl)benzyl)-5,5-dimethyl-1-(methylsulfonyl)imidazolidine-2,4-dione (5 mg, yield: 10.8%) as yellow oil. LCMS purity (220 nm): 81.8%. ¹H NMR (400 MHz, CHCl₃-d) δ=7.31-7.28 (m, 2H), 7.20-7.14 (m, 2H), 7.14-7.11 (m, 2H), 4.71-4.65 (m, 2H), 4.27 (t, J=6.4 Hz, 2H), 3.86 (t, J=6.4 Hz, 2H), 3.38 (s, 3H), 1.76-1.71 (m, 6H), 1.64 (s, 6H). LCMS (M+H⁺) m/z: calcd: 526, found: 527.

For synthesis of Compounds in Tables C, see WO 2019/226991 for procedures. The disclosures of WO 2019/226991 are hereby incorporated by reference in their entireties.

### Example 18: (S)—N-(3-(4-(2-(3,5-dichloro-4-(3-chloro-2-hydroxypropoxy)phenyl) propan-2-yl)phenoxy)-2-oxopropyl)methanesulfonamide (AA51(S))

To a solution of (R)—N-(3-(4-(2-(3,5-dichloro-4-(oxiran-2-ylmethoxy)phenyl)propan-2-yl)phenoxy)-2-oxopropyl)methanesulfonamide (1g) (30 mg, 0.06 mmol, 1.0 eq.) in MeCN (6 mL) was added CeCl3.7H₂O (34 mg, 0.09 mmol, 1.5 eq.) and the solution was heated to reflux for 16 hours. The resulting white paste was collected by filtration and washed with ethyl acetate and the clear suspension was concentrated under reduced pressure. The resulting residue was purified by flash silica gel column chromatography (elution: ethyl acetate in hexane) to provide (S)—N-(3-(4-(2-(3,5-dichloro-4-(3-chloro-2-hydroxypropoxy)phenyl) propan-2-yl)phenoxy)-2-oxopropyl)methanesulfonamide (AA51(S)): (13.7 mg, 42.4%) as a colorless oil. LRMS (M+Na⁺) m/z: calcd 560.05; found 560.0. ¹HNMR (400 MHz, DMSO-d6): δ 7.44 (t, J=5.6 Hz, 1H), 7.23 (s, 2H), 7.15 (d, J=8.8 Hz, 2H), 6.84 (d, J=8.8 Hz, 2H), 5.55 (d, J=5.2 Hz, 1H), 4.91 (s, 2H), 4.01-4.10 (m, 3H), 3.96 (d, J=5.6 Hz, 2H), 3.82 (dd, J=4.0, 11.2 Hz, 2H), 3.70 (dd, J=4.0, 11.2 Hz, 2H), 2.93 (s, 3H), 1.60 (s, 6H).

### Example 19: N-(3-(4-(2-(3,5-dichloro-4-(3-chloropropoxy)phenyl)propan-2-yl)phenoxy)-2-oxopropyl)methanesulfonamide (AA31)

To a solution of (R)—N-(3-(4-(2-(3,5-dichloro-4-(3-CHloropropoxy)phenyl)propan-2-yl)phenoxy)-2-hydroxypropyl)methanesulfonamide (2a) (25.0 mg, 0.048 mmol, 1.0 eq.) in anhydrous dichloromethane (3 mL) was treated Dess-Martin periodinane (41 mg, 0.096 mmol, 2.0 eq.) at 0° C. for 10 minutes. Then it was warmed to the room temperature for 16 hours. The reaction was quenched by the addition of a saturated solution of ammonium chloride (2 ml) and the mixture was extracted with ethyl acetate (2×30 ml). The combined organic layers were washed with deionized water (2×30 ml), dried over anhydrous magnesium sulfate, filtered, and concentrated under reduced pressure. The resulting residue was purified by gradient flash silica gel column chromatography (elution: acetate in hexane) to provide N-(3-(4-(2-(3,5-dichloro-4-(3-chloropropoxy)phenyl)propan-2-yl)phenoxy)-2-oxopropyl)methanesulfonamide (AA31) (30 mg, 88% yield) as a colorless oil. LRMS (M+Na⁺) m/z: calcd 544.06; found 544.2. 1HNMR (400 MHz, DMSO-d6): δ 7.44 (t, J=5.6 Hz, 1H), 7.24 (s, 2H), 7.15 (d, J=8.8 Hz, 2H), 6.85 (d, J=8.8 Hz, 2H), 4.91 (s, 2H), 4.01 (m, 4H), 3.86 (t, J=6.4 Hz, 2H), 2.93 (s, 3H), 2.19 (m, 2H), 1.60 (s, 6H).

### Example 20: 1-(4-(2-(3,5-dichloro-4-(3-chloropropoxy)phenyl)propan-2-yl)phenoxy)-3-(methylsulfonyl)propan-2-one (AA55)

Compound (AA55) was synthesized according to Compound (AA31) by using (S)-2,6-dichloro-4-(2-(4-(2-hydroxy-3-(methylsulfonyl)propoxy)phenyl)propan-2-yl)phenol (3d) Yield (94.1%). LRMS (M+Na⁺) m/z: calcd 529.06; found 529.3. ¹HNMR (400 MHz, DMSO-d6): δ 7.24 (s, 2H), 7.15 (d, J=9.2 Hz, 2H), 6.84 (d, J=8.8 Hz, 2H), 4.96 (s, 2H), 4.59 (s, 2H), 4.08 (t, J=6.0 Hz, 2H), 3.86 (t, J=6.0 Hz, 2H), 3.11 (s, 3H), 2.19 (m, 2H), 1.61 (s, 6H).

### Example 21: N-(3-(4-(3-(3,5-dichloro-4-(3-chloropropoxy)phenyl)oxetan-3-yl)phenoxy)-2-oxopropyl)methanesulfonamide (AA43)

To a solution of tert-butyl N-(3-(4-(3-(3,5-dichloro-4-(3-chloropropoxy)phenyl)oxetan-3-yl)phenoxy)-2-oxo-propyl)-N-methylsulfonyl-carbamate (60 mg, 0.1 mmol) in DCM (2 mL) was added formic acid (1 mL) and the solution was stirred at 25° C. for 15 min. TLC showed the reaction was completed. The reaction was concentrated under reduced pressure. The residue was purified by prep-HPLC (HCO2H) to give N-(3-(4-(3-(3,5-dichloro-4-(3-chloropropoxy)phenyl)oxetan-3-yl)phenoxy)-2-oxopropyl)methanesulfonamide (6.7 mg, yield: 13.2%) as colorless oil. LCMS purity (220 nm): 94.5%. ¹H NMR (400 MHz, CHCl₃-d) δ 7.18-7.10 (m, 4H), 6.93 (br d, J=7.7 Hz, 2H), 5.20 (br d, J=5.1 Hz, 2H), 5.12 (br d, J=5.3 Hz, 2H), 5.05 (br s, 1H), 4.69 (s, 2H), 4.41 (br d, J=4.2 Hz, 2H), 4.21-4.14 (m, 2H), 3.87 (br t, J=5.8 Hz, 2H), 3.01 (s, 3H), 2.30 (br t, J=5.8 Hz, 2H). %). LRMS (M+H⁺) m/z: calcd 535.0; found 535.

### Example 22: Synthesis of N-(4-(2-(3,5-dichloro-4-(3-chloropropoxy)phenyl)propan-2-yl)benzyl)-2-(methylsulfonamido)acetamide (AA46)

To a solution of 2-bromo-N-[[4-[1-[3,5-dichloro-4-(3-chloropropoxy)phenyl]-1-methyl-ethyl]phenyl]methyl]acetamide (5) (100 mg, 0.20 mol) and Cs₂CO₃ (321 mg, 0.98 mmol) in DMF (5 mL) was added methanesulfonamide (37.5 mg, 0.39 mmol). Then the resulting solution was stirred at 25° C. for 2 hours. LCMS showed the reaction was completed. The solution was poured into water (5 mL) and the organic layer was separated. The aqueous phase was extracted with EtOAc (3 mL×4). The combined organic layers were washed with brine (4 mL×3), dried over Na₂SO₄, filtered and concentrated. The crude product was purified by prep-HPLC (TFA) to give the N-[[4-[1-[3,5-dichloro-4-(3-chloropropoxy)phenyl]-1-methyl-ethyl]phenyl]methyl]-2-(methanesulfonamido)acetamide (24.1 mg, yield: 23.4%) as a yellow gum. HPLC purity (220 nm): 98.3%. ¹H NMR (400 MHz, CHCl₃-d) δ 7.26-7.18 (m, 4H), 7.14 (s, 2H), 6.34 (br s, 1H), 5.02 (br s, 1H), 4.49 (d, J=5.7 Hz, 2H), 4.18 (t, J=5.8 Hz, 2H), 3.92-3.85 (m, 4H), 3.03 (s, 3H), 2.31 (quin, J=6.1 Hz, 2H), 1.66 (s, 6H). LCMS (M+H⁺) m/z: clcd 522.1; found 523.0.

### Example 23: Synthesis of N-(3,5-dichloro-4-(3-chloropropoxy)phenyl)-N-(4-(3-(methylsulfonamido)-2-oxopropoxy)phenyl)acetamide (AA71)

A solution of tert-butyl (3-(4-(N-(3,5-dichloro-4-(3-chloropropoxy)phenyl)acetamido)phenoxy)-2-oxopropyl)(methylsulfonyl)carbamate (200 mg, 0.2 mmol) in HCl/EtOAc (4 M, 4 mL) was stirred at 25° C. for 15 min. TLC showed the reaction was completed. The reaction was concentrated under reduced pressure. The residue was purified by prep-HPLC (HCl) to give N-(3,5-dichloro-4-(3-chloropropoxy)phenyl)-N-(4-(3-(methylsulfonamido)-2-oxopropoxy)phenyl)acetamide (69 mg, yield: 59.0%) as yellow oil. HPLC purity (220 nm): 93.5%. ¹H NMR (400 MHz, CHCl₃-d) δ=7.26-7.21 (m, 4H), 7.01-6.92 (m, 2H), 5.05 (br s, 1H), 4.70 (s, 2H), 4.40 (d, J=5.1 Hz, 2H), 4.15 (br s, 2H), 3.85 (t, J=6.4 Hz, 2H), 3.02 (s, 3H), 2.28 (quin, J=6.0 Hz, 2H), 2.10-2.01 (m, 3H). LCMS (M+H⁺) m/z: clcd: 538.0; found: 539.0.

### Example 24: Synthesis of N-(3-((3′,5′-dichloro-4′-(3-chloropropoxy)-[1,1′-biphenyl]-4-yl)oxy)-2-oxopropyl)methanesulfonamide (AA73)

A solution of tert-butyl (3-((3′,5′-dichloro-4′-(3-chloropropoxy)-[1,1′-biphenyl]-4-yl)oxy)-2-oxopropyl)(methylsulfonyl)carbamate (7) (70.0%, 0.13 g, 0.15 mol) in HCl/EtOAc (2 mL) was stirred at 20° C. for 0.5 hour. LCMS showed the reaction was completed. The solution was concentrated under reduced pressure. The crude product was purified by prep-HPLC (HCl) to give N-(3-((3′,5′-dichloro-4′-(3-chloropropoxy)-[1,1′-biphenyl]-4-yl)oxy)-2-oxopropyl)methanesulfonamide (29 mg, yield: 27.0%) as brown oil. HPLC purity (220 nm): 90.5%. ¹H NMR (400 MHz, CHCl₃-d) δ=7.51-7.48 (d, J=8.8 Hz, 2H), 7.47 (s, 2H), 7.00-6.96 (d, J=8.6 Hz, 2H), 5.12-4.99 (m, 1H), 4.72 (s, 2H), 4.46-4.37 (d, J=5.2 Hz, 2H), 4.25-4.17 (t, J=5.7 Hz, 2H), 3.96-3.83 (t, J=6.4 Hz, 2H), 3.02 (s, 3H), 2.38-2.25 (m, 2H). LCMS (M+H⁺) m/z: clcd: 480.0; found 480.0.

### Example 25: Synthesis of N-(3-(4-(3,5-dichloro-4-(3-chloro-2-hydroxypropoxy)phenethyl)phenoxy)-2-oxopropyl)methanesulfonamide (AA75)

A solution of tert-butyl N-(3-(4-(2-(3,5-dichloro-4-(3-chloro-2-(methoxymethoxy)propoxy)phenyl)ethyl)phenoxy)-2-oxo-propyl)-N-methylsulfonyl-carbamate (9) (180 mg, 0.27 mmol) in TFA (2 mL) and DCM (10 mL) was stirred at 20° C. for 3 hours. LCMS showed the reaction was completed. The resulting solution was concentrated under reduced pressure. The residue was purified by prep-HPLC (HCl) to give N-(3-(4-(2-(3, 5-dichloro-4-(3-chloro-2-hydroxy-propoxy)phenyl)ethyl)phenoxy)-2-oxo-propyl)methanesulfonamide (13.9 mg, yield: 9.84%) as white solid. HPLC purity (220 nm): 92%. ¹H NMR (400 MHz, CHCl3-d) δ ppm 7.06-7.11 (m, 4H), 6.80-6.86 (m, 2H), 5.02 (br s, 1H), 4.63-4.69 (m, 2H), 4.40 (d, J=5.14 Hz, 2H), 4.23 (br s, 1H), 4.12-4.20 (m, 2H), 3.74-3.90 (m, 2H), 3.00 (s, 3H), 2.83-2.86 (m, 2H), 2.79-2.83 (m, 2H).

### Example 26: Synthesis of N-(3-((4-(2-(3,5-dichloro-4-(2-chloroethoxy)phenyl)propan-2-yl)phenyl) (methyl)amino)-2-oxopropyl)methanesulfonamide hydrochloride (AA81)

A solution of tert-butyl (3-((4-(2-(3,5-dichloro-4-(2-chloroethoxy)phenyl)propan-2-yl)phenyl)(methyl)amino)-2-oxopropyl) (methylsulfonyl)carbamate (100 mg, 0.2 mmol) in HCl/EtOAc (2 mL) was stirred at 25° C. for 15 min. TLC showed the reaction was completed. The reaction was concentrated under reduced pressure. The residue was purified by prep-HPLC (HCl) to give N-(3-((4-(2-(3,5-dichloro-4-(2-chloroethoxy)phenyl)propan-2-yl)phenyl)(methyl)amino)-2-oxopropyl)methanesulfonamide hydrochloride (7.6 mg, yield: 9.1%) as yellow oil. ¹H NMR (400 MHz, CHCl₃-d) δ 7.16 (br s, 2H), 7.12 (s, 2H), 6.95 (br s, 2H), 5.70 (br s, 1H), 4.49-4.22 (m, 4H), 4.15-3.81 (m, 4H), 3.18 (br s, 3H), 2.94 (br s, 3H), 1.62 (s, 6H). LCMS (M+H⁺) m/z: clcd: 520.1; found 521.0.

For synthesis of Compounds in Tables D, see WO 2017/177307 for procedures. The disclosures of WO 2017/177307 are hereby incorporated by reference in their entireties.

### Example 27: Synthesis of (R)-3-(4-(2-(3,5-dichloro-4-((S)-3-chloro-2-hydroxypropoxy)phenyl)propan-2-yl)phenoxy)propane-1,2-diol (Compound 1a)

To a solution of (S)-4-((4-(2-(3,5-dichloro-4-(((R)-oxiran-2-yl)methoxy)phenyl)propan-2-yl)phenoxy)methyl)-2,2-dimethyl-1,3-dioxolane (560 mg, 1.2 mmol, 1.0 equiv) in MeCN (12 mL) was added CeCl₃.7H₂O (1118 mg, 3.0 mmol, 2.5 equiv) and the mixture was heated to reflux for 16 h. The resulting white paste was collected by filtration and washed with ethyl acetate and the clear suspension was concentrated under reduced pressure. The resulting residue was purified by column chromatography to provide the titled compound (512 mg, 92%) as a sticky oil. ¹H NMR (600 MHz, CDCl₃) δ (ppm)=7.15-7.12 (m, 4H), 6.86 (d, J=9.0 Hz, 2H), 4.26-4.23 (m, 1H), 4.21-4.15 (m, 2H), 4.15-4.11 (m, 1H), 4.08-4.03 (m, 2H), 3.86 (dd, J=4.8 Hz, 10.8 Hz, 2H), 3.78 (dd, J=6.6 Hz, 12.6 Hz, 2H), 1.64 (s, 6H); ¹³C NMR (150 MHz, CDCl₃) δ (ppm)=156.76, 149.30, 148.26, 141.84, 128.52, 127.87, 127.67, 114.35, 73.69, 70.48, 69.26, 63.78, 45.55, 42.34, 30.79; ESI-LRMS calcd for [M+Na]⁺485.1, found 485.4.

### Example 28: Synthesis of (R)-3-(4-(2-(3,5-dibromo-4-((S)-3-chloro-2-hydroxypropoxy)phenyl)propan-2-yl)phenoxy)propane-1,2-diol (Compound 3a)

Compound 3a was synthesized by a similar procedure used to prepare Compound 1a in Example 27. ¹H NMR (400 MHz, DMSO-D6) δ (ppm)=7.39 (s, 1H), 7.30 (dd, J=2.0 Hz, 34.4 Hz, 1H), 7.15 (d, J=8.8 Hz, 2H), 6.86 (d, J=8.8 Hz, 2H), 5.57-5.54 (m, 1H), 4.91 (d, J=4.8 Hz, 1H), 4.64 (t, J=5.6 Hz, 1H), 4.10-4.08 (m, 1H), 3.98-3.92 (m, 3H), 3.86-3.81 (m, 2H), 3.79-3.76 (m, 1H), 3.71 (dd, J=5.6 Hz, 11.2 Hz, 1H), 3.45-3.42 (m, 2H), 1.60 (s, 6H).

### Example 29: Synthesis of (S)-1-chloro-3-(2,6-dichloro-4-(2-(4-((R)-2-hydroxy-3-methoxypropoxy)phenyl)propan-2-yl)phenoxy)propan-2-ol (Compound 5a)

To a solution of (S)-1-chloro-3-(2,6-dichloro-4-(2-(4-(((R)-oxiran-2-yl)methoxy)phenyl)propan-2-yl)phenoxy)propan-2-ol (15 mg, 0.034 mmol, 1.0 equiv) in anhydrous methanol (2 mL) was added Erbium (III) trifluoromethanesulfonate (2.1 mg, 0.0034 mmol, 0.1 equiv) and the mixture was stirred at room temperature for 40 h. The reaction was quenched by the addition of a saturated solution of ammonium chloride (0.5 ml) and the mixture was extracted with ethyl acetate (2×10 ml). The organic layer was washed with deionized water (2×10 ml), dried over anhydrous magnesium sulfate, filtered, and concentrated under reduced pressure. The resulting residue was purified by gradient flash column chromatography on silica gel (elution: 30% ethyl acetate in hexane to 50% ethyl acetate in hexane) to provide Compound 5a (12.5 mg, 77.1%) as a colorless oil. ¹H NMR (600 MHz, CDCl₃) δ (ppm)=7.14-7.10 (m, 4H), 6.87 (d, J=6.0 Hz, 2H), 4.26-4.22 (m, 1H), 4.21-4.15 (m, 3H), 4.06-4.01 (m, 2H), 3.87 (dd, J=6.0 Hz, 11.4 Hz, 1H), 3.79 (dd, J=5.4 Hz, 11.4 Hz, 1H), 3.61 (dd, J=4.2 Hz, 9.6 Hz, 1H), 3.57 (dd, J=6.0 Hz, 9.6 Hz, 1H), 3.44 (s, 3H), 1.64 (s, 6H); ¹³C NMR (150 MHz, CDCl₃) δ (ppm)=156.37, 148.81, 147.69, 141.04, 127.95, 127.25, 127.05, 113.81, 73.13, 73.00, 69.93, 68.58, 68.44, 58.88, 45.00, 41.78, 30.25.

### Example 30: Synthesis of (S)-1-chloro-3-(2,6-dichloro-4-(2-(4-((R)-2-hydroxy-3-isopropoxypropoxy)phenyl)propan-2-yl)phenoxy)propan-2-ol (Compound 7a)

Compound 7a was synthesized by a similar procedure used to prepare Compound 3a in Example 28. ¹H NMR (400 MHz, CDCl₃) δ (ppm)=7.13-7.10 (m, 4H), 6.86 (d, J=8.8 Hz, 2H), 4.25-4.12 (m, 4H), 4.03-3.98 (m, 2H), 3.85 (dd, J=5.2 Hz, 10.8 Hz, 1H), 3.77 (dd, J=5.6 Hz, 11.2 Hz, 1H), 3.67-3.53 (m, 3H), 2.83 (s, 1H), 2.57 (s, 1H), 1.62 (s, 6H), 1.18 (d, J=6.0 Hz, 6H).

### Example 31: Synthesis of (S)-1-chloro-3-(2,6-dichloro-4-(2-(4-((S)-3-fluoro-2-hydroxypropoxy)phenyl)propan-2-yl)phenoxy)propan-2-ol (Compound 8a)

To a solution of Compound 1a (1 equiv; synthesized according to Example 27) in dichloromethane were successively added triethylamine trihydrofluoride (2 equiv) and XtalFluor-M (2 equiv). After 3 h, the reaction mixture was quenched at room temperature with a 5% aqueous sodium bicarbonate solution and stirred for 15 min, and the resulting mixture was extracted twice with dichloromethane. The organic phases were combined, dried over anhydrous magnesium sulfate, and filtered. Solvents were evaporated, and the resulting crude material was purified by silica gel chromatography to provide Compound 8a. ¹H NMR (600 MHz, CDCl₃) δ (ppm)=7.16-7.14 (m, 4H), 6.87 (d, J=8.4 Hz, 2H), 4.69-4.56 (m, 2H), 4.30-4.22 (m, 2H), 4.22-4.16 (m, 2H), 4.10-4.09 (m, 2H), 3.87 (dd, J=6.0 Hz, 11.4 Hz, 1H), 3.79 (dd, J=5.4 Hz, 10.8 Hz, 1H), 1.64 (s, 6H).

### Example 32: Synthesis of (S)-1-chloro-3-(2,6-dichloro-4-(2-(4-((R)-2-hydroxy-3-(1H-imidazol-1-yl)propoxy)phenyl)propan-2-yl)phenoxy)propan-2-ol (Compound 9a)

To a solution of (S)-1-chloro-3-(2,6-dichloro-4-(2-(4-(((R)-oxiran-2-yl)methoxy)phenyl)propan-2-yl)phenoxy)propan-2-ol (12.6 mg, 0.028 mmol, 1.0 equiv) in anhydrous MeCN (2 mL) was added Bismuth (III) trifluoromethanesulfonate (1.8 mg, 0.0028 mmol, 0.1 equiv) and the mixture was stirred at room temperature for 40 h. The reaction was quenched by the addition of a saturated solution of ammonium chloride (0.5 ml) and the mixture was extracted with ethyl acetate (2×10 ml). The organic layer was washed with deionized water (2×10 ml), dried over anhydrous magnesium sulfate, filtered, and concentrated under reduced pressure. The resulting residue was purified by flash column chromatography to provide Compound 9a (8.7 mg, 60.4%) as a colorless oil. ¹H NMR (600 MHz, CDCl₃) δ (ppm)=7.56 (s, 1H), 7.16-7.14 (m, 4H), 7.04 (s, 1H), 7.01 (s, 1H), 6.86 (d, J=8.4 Hz, 2H), 4.29-4.23 (m, 3H), 4.22-4.13 (m, 3H), 3.98-3.92 (m, 2H), 3.87 (dd, J=6.0 Hz, 11.4 Hz, 1H), 3.79 (dd, J=4.8 Hz, 10.8 Hz, 1H), 1.65 (s, 6H).

### Example 33: Synthesis of (S)-1-chloro-3-(2,6-dichloro-4-(2-(4-((R)-2-hydroxy-3-morpholinopropoxy)phenyl)propan-2-yl)phenoxy)propan-2-ol (Compound 11a)

Compound 11a was synthesized by a similar procedure used to prepare Compound 9a in Example 32. ¹H NMR (400 MHz, CDCl₃) δ (ppm)=7.16-7.11 (m, 4H), 6.88 (d, J=8.8 Hz, 2H), 4.27-4.13 (m, 4H), 4.07-3.98 (m, 2H), 3.90-3.77 (m, 6H), 2.84-2.80 (m, 2H), 2.73-2.72 (m, 2H), 2.71-2.67 (m, 2H), 1.65 (s, 6H); ESI-LRMS calcd for [M+H]⁺ 532.1, found 534.6.

### Example 34: Synthesis of (R)-1-amino-3-(4-(2-(3,5-dichloro-4-((S)-3-chloro-2-hydroxypropoxy)phenyl)propan-2-yl)phenoxy)propan-2-ol (Compound 12a) and N—((R)-3-(4-(2-(3,5-dichloro-4-((S)-3-chloro-2-hydroxypropoxy)phenyl)propan-2-yl)phenoxy)-2-hydroxypropyl)methanesulfonamide (Compound 13a)

Synthesis of (R)-1-amino-3-(4-(2-(3,5-dichloro-4-((S)-3-chloro-2-hydroxypropoxy)phenyl)propan-2-yl)phenoxy)propan-2-ol (Compound 12a). To a solution of (R)-1-azido-3-(4-(2-(3,5-dichloro-4-((S)-3-chloro-2-hydroxypropoxy)phenyl)propan-2-yl)phenoxy)propan-2-ol (57 mg, 0.117 mmol, 1.0 equiv) in MeCN (6 mL) was added triphenylphosphine (36.7 mg, 0.14 mmol, 1.2 equiv) and the mixture was heated to reflux for 16 h. The reaction was quenched by deionized water (2 ml) and the mixture was extracted with ethyl acetate (2×30 ml). The organic layer was washed with deionized water (2×30 ml), dried over anhydrous magnesium sulfate, filtered, and concentrated under reduced pressure. The resulting residue was purified by gradient flash column chromatography on Si gel (elution: 2% methanol in dichloromethane to 30% methanol in dichloromethane) to provide Compound 12a (24.3 mg, 44.9%) as a colorless oil. ¹H NMR (400 MHz, CDCl₃) δ (ppm)=7.12-7.09 (m, 4H), 6.84 (d, J=8.4 Hz, 2H), 4.24-4.21 (m, 1H), 4.17-4.13 (m, 2H), 3.97 (m, 3H), 3.84 (dd, J=5.6 Hz, 11.2 Hz, 1H), 3.76 (dd, J=5.6 Hz, 11.2 Hz, 1H), 3.00-2.85 (m, 2H), 1.61 (s, 6H); ¹³C NMR (100 MHz, CDCl₃) δ (ppm)=157.00, 149.35, 148.31, 141.60, 128.52, 127.81, 127.61, 114.37, 73.78, 70.47, 70.42, 70.14, 45.65, 44.11, 42.34, 30.25; ESI-LRMS calcd for [M+H]⁺ 462.1, found 463.9.

Synthesis of N—((R)-3-(4-(2-(3,5-dichloro-4-((S)-3-chloro-2-hydroxypropoxy)phenyl)propan-2-yl)phenoxy)-2-hydroxypropyl)methanesulfonamide (Compound 13a). To a solution of (R)-1-azido-3-(4-(2-(3,5-dichloro-4-((S)-3-chloro-2-hydroxypropoxy)phenyl)propan-2-yl)phenoxy)propan-2-ol (14.3 mg, 0.031 mmol, 1.0 equiv) in anhydrous dichloromethane (3 mL) was treated triethylamine (12.5 mg, 0.124 mmol, 4.0 equiv) and methane sulfonyl chloride (3.6 mg, 0.031 mmol, 1.0 equiv) sequentially at 0° C. for 10 minutes. Then it was warmed to room temperature for 16 hours. The reaction was quenched by the addition of a saturated solution of ammonium chloride (2 ml) and the mixture was extracted with ethyl acetate (2×20 ml). The organic layer was washed with deionized water (2×20 ml), dried over anhydrous magnesium sulfate, filtered, and concentrated under reduced pressure. The resulting residue was purified by gradient flash column chromatography on silica gel (elution: 50% ethyl acetate in hexane to 75% ethyl acetate in hexane) to provide Compound 13a (9.7 mg, 57.9%) as a colorless oil. ¹H NMR (600 MHz, CDCl₃) δ (ppm)=7.15-7.13 (m, 4H), 6.87 (d, J=5.4 Hz, 2H), 4.93-4.90 (m, 1H), 4.26-4.23 (m, 1H), 4.21-4.13 (m, 3H), 4.06-4.01 (m, 2H), 3.87 (dd, J=5.4 Hz, 11.4 Hz, 1H), 3.79 (dd, J=5.4 Hz, 10.8 Hz, 1H), 3.50-3.45 (m, 1H), 3.26-3.31 (m, 1H), 3.03 (s, 3H), 1.64 (s, 6H); ¹³C NMR (150 MHz, CDCl₃) δ (ppm)=155.94, 148.69, 147.73, 141.57, 127.99, 127.39, 127.05, 113.81, 73.14, 69.92, 68.85, 68.54, 45.19, 44.99, 41.81, 39.98, 30.22.

### Example 35: Synthesis of (S)-1-chloro-3-(2,6-dichloro-4-(2-(4-((S)-3-(ethylsulfonyl)-2-hydroxypropoxy)phenyl)propan-2-yl)phenoxy)propan-2-ol (Compound 14a)

To a solution of (S)-1-chloro-3-(2,6-dichloro-4-(2-(4-((S)-3-(ethylthio)-2-hydroxypropoxy)phenyl)propan-2-yl)phenoxy)propan-2-ol (14.6 mg, 0.029 mmol, 1.0 equiv) in anhydrous dichloromethane (3 mL) was treated 3-chloroperbenzoic acid (14.0 mg, 0.081 mmol, 2.8 equiv) at 0° C. for 10 minutes. Then it was warmed to room temperature for 3 hours. The reaction was quenched by the addition of a saturated solution of ammonium chloride (2 ml) and the mixture was extracted with ethyl acetate (2×20 ml). The organic layer was washed with saturated NaHCO₃ (20 ml), deionized water (2×20 ml), dried over anhydrous magnesium sulfate, filtered, and concentrated under reduced pressure. The resulting residue was purified by gradient flash column chromatography on Si gel (elution: 30% ethyl acetate in hexane to 75% ethyl acetate in hexane) to provide Compound 14a (4.7 mg, 31.0%) as a colorless oil. ¹H NMR (400 MHz, CDCl₃) δ (ppm)=7.18-7.15 (m, 4H), 6.88 (d, J=8.8 Hz, 2H), 4.69-4.67 (m, 1H), 4.27-4.15 (m, 3H), 4.10-4.07 (m, 2H), 3.88 (dd, J=5.2 Hz, J=11.2 Hz, 1H), 3.80 (dd, J=5.2 Hz, J=10.8 Hz, 1H), 3.39-3.20 (m, 4H), 1.66 (s, 6H), 1.48 (t, J=7.2 Hz, 3H); ESI-LRMS calcd for [M+Na]⁺561.1, found 561.5.

### Example 36: Synthesis of (R)-3-(4-(2-(4-((S)-3-chloro-2-hydroxypropoxy)-3-methylphenyl)propan-2-yl)-2-methylphenoxy)propane-1,2-diol (Compound 22a)

Compound 22a was synthesized by a similar procedure used to prepare Compound 1a in Example 27. ¹H NMR (400 MHz, DMSO-D6) δ (ppm)=6.97-6.94 (m, 4H), 6.81-6.76 (m, 2H), 5.50 (d, J=4.8 Hz, 1H), 4.86 (s, 1H), 4.61 (s, 1H), 4.06-4.00 (m, 1H), 3.97-3.89 (m, 3H), 3.86-3.76 (m, 3H), 3.69 (dd, J=5.6 Hz, 11.2 Hz, 1H), 3.50-3.44 (m, 2H), 2.10 (s, 6H), 1.55 (s, 6H); ¹³C NMR (100 MHz, DMSO-D₆) δ (ppm)=155.14, 154.74, 143.23, 142.75, 129.32, 129.23, 125.66, 125.62, 125.22, 125.16, 111.28, 111.16, 70.67, 70.02, 69.48, 69.32, 63.43, 55.50, 47.53, 31.42, 16.86, 16.79.

### Example 37: Synthesis (R)-1-(4-(2-(4-((R)-2-acetoxy-3-chloropropoxy)-3,5-dichlorophenyl)propan-2-yl)phenoxy)-3-methoxypropan-2-yl acetate (Compound 5dA)

Ac₂O (128 mg, 1.26 mmol, 6.0 equiv.), Et3N (127 mg, 1.26 mmol, 6.0 equiv.) and DMAP (26 mg, 0.21 mmol, 1.0 equiv.) were added to a solution of Compound 5a (100 mg, 0.21 mmol, 1.0 equiv., see Example 29) in anhydrous DCM (5 mL) at room temperature and the resultant mixture was stirred at the same temperature overnight. The mixture was diluted with EtOAc (30 mL) and the organic layer was washed with water (15 mL) and brine (15 mL). The organic layer was further dried over anhydrous MgSO₄ and evaporated under reduced pressure. The crude was loaded onto a silica gel column and eluted with Hexane/EtOAc (13/1 to 6/1) to give 111 mg of the titled compound as colorless oil (yield: 95.0%). ¹H NMR (600 MHz, CHLOROFORM-d) δ 7.11-7.12 (m, 2H), 7.09-7.11 (m, 2H), 6.82-6.87 (m, 2H), 5.32-5.35 (m, 1H), 5.28-5.32 (m, 1H), 4.18-4.26 (m, 2H), 4.09-4.16 (m, 2H), 3.97 (dd, J=5.14, 11.74 Hz, 1H), 3.88 (dd, J=5.14, 11.74 Hz, 1H), 3.66 (dd, J=2.20, 4.40 Hz, 2H), 3.40 (s, 3H), 2.14 (s, 3H), 2.11 (s, 3H), 1.61 (s, 6H). ¹³C NMR (151 MHz, CHLOROFORM-d) δ 170.8, 170.4, 156.9, 149.4, 148.4, 141.8, 128.7, 127.9, 127.7, 114.5, 71.9, 71.1, 70.9, 66.4, 59.6, 42.7, 42.4, 30.9, 21.4, 21.2.

### Example 38: Synthesis (R)-1-(4-(2-(4-((S)-2-acetoxy-3-chloropropoxy)-3,5-dichlorophenyl)propan-2-yl)phenoxy)-3-methoxypropan-2-yl acetate (Compound 5aA)

Acetic Anhydride (4.1 mg, 0.04 mmol, 4.0 equiv) was added to a solution of Compound 5a (5.0 mg, 0.01 mmol, 1.0 equiv, see Example 29), DMAP (0.1 mg, 0.001 mmol, 0.1 equiv) and Et3N (4.1 mg, 0.04 mmol, 4.0 equiv) in anhydrous dichloromethane (1 mL). The resulting solution was stirred overnight at room temperature. Dichloromethane was removed under reduced pressure and the residue was purified by column chromatography to afford the title compound as a colorless oil (5.8 mg, 98.6%). ¹H NMR (400 MHz, CDCl₃) δ (ppm)=7.11-7.08 (m, 4H), 6.83 (d, J=8.8, 2H), 5.35-5.26 (m, 2H), 4.26-4.17 (m, 2H), 4.16-4.07 (m, 2H), 3.96 (dd, J=5.2 Hz, 11.6 Hz, 1H), 3.86 (dd, J=5.6 Hz, 11.6 Hz, 1H), 3.66-3.61 (m, 2H), 3.38 (s, 3H), 2.13 (s, 3H), 2.10 (s, 3H), 1.60 (s, 6H); ¹³C NMR (150 MHz, CDCl₃) δ (ppm)=170.80, 170.45, 156.96, 149.41, 148.39, 141.80, 128.69, 127.90, 127.70, 114.54, 71.91, 71.12, 70.54, 66.44, 59.62, 42.73, 42.43, 30.90, 21.38, 21.18; ESI-LRMS calcd for [M+H]⁺ 561.1, found 561.1.

### Example 39: Synthesis of (R)-1-(4-(2-(4-((S)-2-acetoxy-3-chloropropoxy)-3,5-dichlorophenyl)propan-2-yl)phenoxy)-3-isopropoxypropan-2-yl acetate (Compound 7aA)

Compound 7aA was synthesized by a similar procedure used to prepare Compound 5aA in Examples 38 by using Compound 7a prepared according to Example 30. Compound 7aA was obtained as a colorless oil (6.4 mg, 96.2%). ¹H NMR (400 MHz, CDCl₃) δ (ppm)=7.12-7.08 (m, 4H), 6.85 (d, J=8.8, 2H), 5.36-5.30 (m, 1H), 5.28-5.22 (m, 1H), 4.27-4.09 (m, 4H), 3.97 (dd, J=5.2 Hz, 11.6 Hz, 1H), 3.87 (dd, J=5.6 Hz, 11.6 Hz, 1H), 3.71-3.57 (m, 3H), 2.14 (s, 3H), 2.09 (s, 3H), 1.61 (s, 6H), 1.15 (dd, J=2.0 Hz, 6.0 Hz, 6H); ¹³C NMR (150 MHz, CDCl₃) δ (ppm)=170.16, 169.78, 156.42, 148.77, 147.72, 141.03, 128.02, 127.20, 127.03, 113.91, 71.97, 71.25, 70.98, 70.30, 66.01, 65.72, 42.06, 41.76, 30.24, 21.60, 21.54, 20.74, 20.52; ESI-LRMS calcd for [M+Na]⁺611.1, found 611.1.

### Example 40: Synthesis of (S)-1-(4-(2-(4-((S)-2-acetoxy-3-(ethylsulfonyl)propoxy)phenyl)propan-2-yl)-2,6-dichlorophenoxy)-3-chloropropan-2-yl acetate (Compound 14aA)

Compound 14aA was synthesized by a similar procedure used to prepare Compound 5aA in Examples 38 by using Compound 14a prepared according to Example 35. Compound 14aA was obtained as a colorless oil (3.4 mg, 97.3%). ¹H NMR (400 MHz, CDCl₃) δ (ppm)=7.13-7.08 (m, 4H), 6.84 (d, J=8.8, 2H), 5.63-5.57 (m, 1H), 5.36-5.30 (m, 1H), 4.29-4.18 (m, 4H), 3.97 (dd, J=5.2 Hz, 12.0 Hz, 1H), 3.87 (dd, J=5.6 Hz, 11.6 Hz, 1H), 3.54-3.40 (m, 2H), 3.10 (q, J=7.2 Hz, 2H), 2.14 (s, 3H), 2.12 (s, 3H), 1.61 (s, 6H), 1.44 (t, J=7.2 Hz, 3H); ¹³C NMR (150 MHz, CDCl₃) δ (ppm)=170.41, 170.16, 158.50, 154.94, 142.86, 142.39, 128.70, 128.03, 127.66, 114.48, 71.87, 71.46, 67.79, 67.05, 52.48, 48.82, 42.69, 42.44, 30.86, 21.15, 20.90, 6.80; ESI-LRMS calcd for [M+Na]⁺645.1, found 645.1.

### Example 41: Synthesis of (R)-1-(4-(2-(4-((S)-2-acetoxy-3-chloropropoxy)-3,5-dichlorophenyl)propan-2-yl)phenoxy)-3-morpholinopropan-2-yl acetate (Compound 11aA)

Compound 11aA was synthesized by a similar procedure used to prepare Compound 5aA in Examples 38 by using Compound 11a prepared according to Example 33. Compound 11aA was obtained as a colorless oil (6.8 mg, 97.6%). ¹H NMR (400 MHz, CDCl₃) δ (ppm)=7.14-7.07 (m, 4H), 6.83 (d, J=8.8, 2H), 5.72-5.70 (m, 1H), 5.36-5.30 (m, 1H), 4.47-4.40 (m, 1H), 4.39-4.32 (m, 1H), 4.29-4.14 (m, 4H), 3.99-3.94 (m, 3H), 3.87 (dd, J=5.6 Hz, 11.6 Hz, 1H), 3.58-3.37 (m, 4H), 2.97 (m, 2H), 2.23 (s, 3H), 2.14 (s, 3H), 1.61 (s, 6H); ¹³C NMR (150 MHz, CDCl₃) δ (ppm)=170.54, 170.37, 156.02, 149.12, 148.39, 142.61, 128.67, 128.06, 127.60, 114.34, 71.81, 70.90, 67.26, 65.89, 63.60, 58.52, 53.17, 52.58, 42.64, 42.40, 30.78, 29.87, 21.56, 21.11; ESI-LRMS calcd for [M+H]⁺ 616.1, found 616.1.

### Example 42: Synthesis of (S)-1-(4-(2-(4-((R)-2-acetoxy-3-(1H-imidazol-1-yl)propoxy)phenyl)propan-2-yl)-2,6-dichlorophenoxy)-3-chloropropan-2-yl acetate (Compound 9aA)

Compound 9aA was synthesized by a similar procedure used to prepare Compound 5aA in Examples 38 by using Compound 9a prepared according to Example 32. Compound 9aA was obtained as a colorless oil (5.6 mg, 93.6%). ¹H NMR (400 MHz, CDCl₃) δ (ppm)=9.40 (s, 1H), 7.39 (s, 1H), 7.20 (s, 1H), 7.14-7.10 (m, 4H), 6.82 (d, J=8.4, 2H), 5.50 (m, 1H), 5.35-5.31 (m, 1H), 4.78-4.70 (m, 2H), 4.27-4.18 (m, 4H), 3.96 (dd, J=5.6 Hz, 11.6 Hz, 1H), 3.87 (dd, J=5.6 Hz, 11.6 Hz, 1H), 2.14 (s, 3H), 2.11 (s, 3H), 1.61 (s, 6H); ¹³C NMR (150 MHz, CDCl₃) δ (ppm)=170.21, 169.67, 155.72, 148.93, 142.61, 136.02, 128.53, 127.97, 127.46, 121.62, 120.13, 114.19, 71.66, 70.76, 69.72, 65.27, 49.70, 42.49, 42.26, 30.65, 20.96, 20.91; ESI-LRMS calcd for [M+H]⁺ 597.1, found 597.1.

### Example 43: Synthesis of (S)-1-(4-(2-(4-((R)-2-acetoxy-3-(N-(methylsulfonyl)acetamido)propoxy)phenyl)propan-2-yl)-2,6-dichlorophenoxy)-3-chloropropan-2-yl acetate (Compound 13aA)

Compound 13aA was synthesized according to Examples 38 by using Compound 13a. Compound 13aA was obtained as a colorless oil (6.0 mg, 100%). ¹H NMR (400 MHz, CDCl₃) δ (ppm)=7.13-7.08 (m, 4H), 6.82 (d, J=8.4, 2H), 5.47-5.42 (m, 1H), 5.36-5.30 (m, 1H), 4.29-4.07 (m, 6H), 3.97 (dd, J=5.2 Hz, 11.6 Hz, 1H), 3.87 (dd, J=5.6 Hz, 11.6 Hz, 1H), 3.33 (s, 3H), 2.44 (s, 3H), 2.14 (s, 3H), 2.10 (s, 3H), 1.61 (s, 6H); ¹³C NMR (150 MHz, CDCl₃) δ (ppm)=171.33, 170.47, 170.29, 156.42, 149.13, 148.29, 142.17, 128.58, 127.88, 127.54, 114.33, 71.74, 70.82, 70.36, 67.19, 46.69, 42.66, 42.57, 42.31, 30.73, 24.49, 21.07, 20.03; ESI-LRMS calcd for [M+H]⁺ 666.1, found 666.1.

### Example 44: Synthesis of (S)-1-(4-(2-(4-((S)-2-acetoxy-3-chloropropoxy)-3,5-dichlorophenyl)propan-2-yl)phenoxy)-3-fluoropropan-2-yl acetate (Compound 8aA)

Compound 8aA was synthesized according to Examples 38 by using Compound 8a prepared according to Example 31. Compound 8aA was obtained as a colorless oil (5.8 mg, 95.9%). ¹H NMR (400 MHz, CDCl₃) δ (ppm)=7.13-7.10 (m, 4H), 6.84 (d, J=8.8 Hz, 2H), 5.39-5.29 (m, 2H), 4.79-4.71 (m, 1H), 4.67-4.59 (m, 1H), 4.27-4.18 (m, 2H), 4.16-4.14 (m, 2H), 3.97 (dd, J=5.2 Hz, 11.6 Hz, 1H), 3.87 (dd, J=5.6 Hz, 11.6 Hz, 1H), 2.14 (s, 3H), 2.13 (s, 3H), 1.61 (s, 6H); ¹³C NMR (150 MHz, CDCl₃) δ (ppm)=170.58, 170.46, 156.65, 149.33, 148.43, 142.14, 128.73, 127.99, 127.70, 114.48, 82.13, 80.99 (d, J=513.0 Hz), 71.92, 70.98, 70.77, 70.64 (d, J=19.5 Hz), 65.19, 65.15 (d, J=6.0 Hz), 42.73, 42.46, 30.91, 21.22, 21.19; ESI-LRMS calcd for [M+H]⁺ 549.1, found 549.1.

### Example 45: Synthesis of (S)-3-(4-(2-(4-((S)-2-acetoxy-3-chloropropoxy)-3,5-dichlorophenyl)propan-2-yl)phenoxy)propane-1,2-diyl diacetate (Compound 1aA)

Compound 1aA was synthesized according to Examples 38 by using Compound 1a prepared according to Example 27. Compound 1aA was obtained as a colorless oil (63.0 mg, 97.1%). ¹H NMR (600 MHz, CDCl₃) δ (ppm)=7.14-7.11 (m, 4H), 6.85 (d, J=12.0 Hz, 2H), 5.39-5.33 (m, 2H), 4.45 (dd, J=4.2 Hz, 12.0 Hz, 1H), 4.32 (dd, J=6.0 Hz, 12.0 Hz, 1H), 4.26 (dd, J=4.8 Hz, 10.2 Hz, 1H), 4.22 (dd, J=4.8 Hz, 10.2 Hz, 1H), 4.13-4.12 (m, 2H), 3.98 (dd, J=5.4 Hz, 12.0 Hz, 1H), 3.89 (dd, J=5.4 Hz, 12.0 Hz, 1H), 2.16 (s, 3H), 2.12 (s, 3H), 2.10 (s, 3H), 1.63 (s, 6H); ¹³C NMR (150 MHz, CDCl₃) δ(ppm)=170.21, 169.90, 169.77, 156.07, 148.66, 147.76, 141.40, 128.04, 127.28, 127.02, 113.88, 71.24, 70.31, 69.30, 65.55, 62.10, 42.05, 41.77, 30.23, 20.56, 20.50, 20.35; ESI-LRMS calcd for [M+Na]⁺611.1, found 611.0.

General Synthesis of Protac

A Protac of formula PLM-LI-PTC, or their pharmaceutically acceptable salts can be prepared by the general approaches described herein, together with synthetic methods known in the art of organic chemistry, or modifications and derivatizations that are familiar to one skilled in the art. Covalent bond between PLM and LI and between PTC and LI can be formed via chemistries commonly known to one skilled in the art, including but not limited to, amide formation, ester formation, carbamate formation, urea formation, ether formation, amine formation and various C—C and C═C bond formations.

In one embodiment, the PTC can have a chemical group suitable as a leaving group and the linker LI has a chemical group suitable as a nucleophile (Scheme 1).

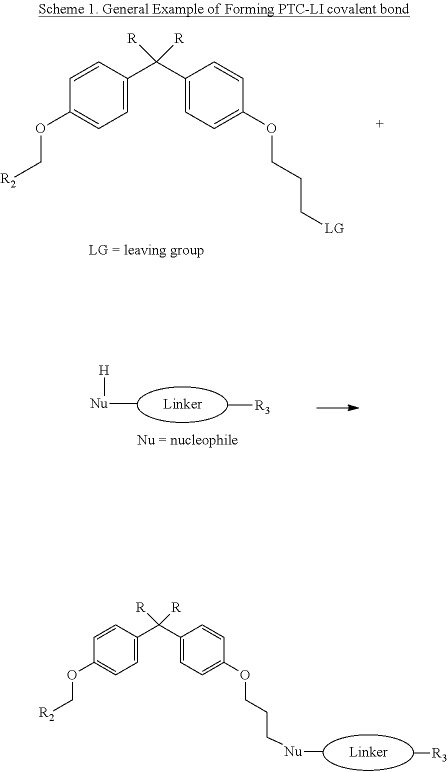

In Scheme 1, LG can be any leaving group commonly known to person skilled in the art, including but not limited to halogen and sulfonates (e.g., tosylate, mesylate). In Scheme 1, Nu-H can be any nucleophile commonly known to person skilled in the art including but not limited to —OH and —NH₂. In Scheme 1, R³ can be a chemical group that would be useful in forming a covalent bond with the PLM. In one embodiment, R³ is protected by a commonly known protecting group such that it does not interfere with the reaction of forming a covalent bond between PTC and LI.

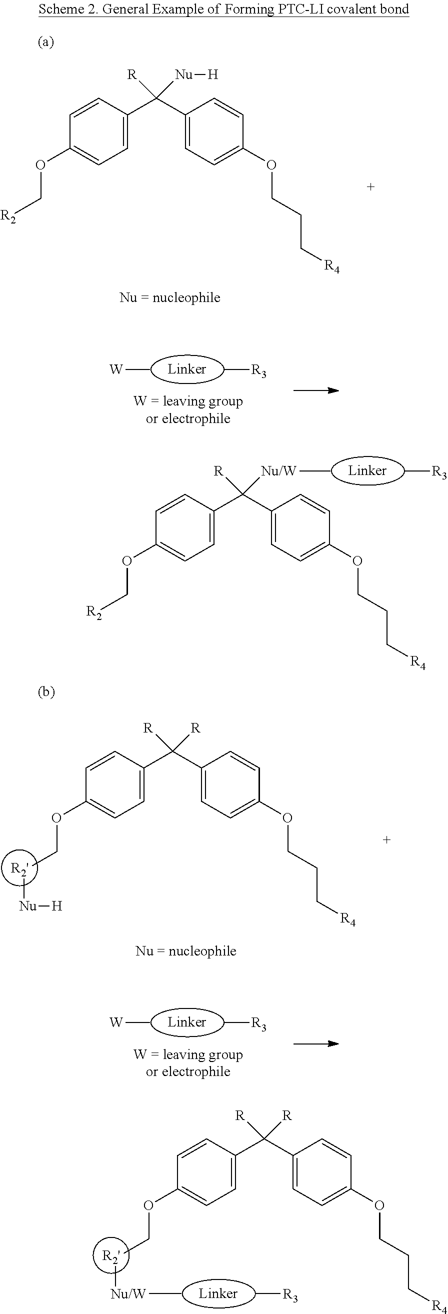

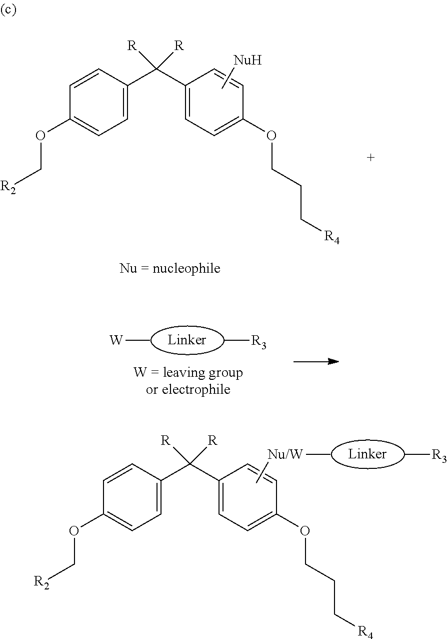

In Scheme 2, LG can be any leaving group commonly known to person skilled in the art, including but not limited to halogen and sulfonates (e.g., tosylate, mesylate). In Scheme 2, electrophile can be any group commonly known to person skilled in the art, including but not limited to carboxylic acid. In Scheme 2, Nu-H can be any nucleophile commonly known to person skilled in the art including but not limited to —OH and —NH₂. In one embodiment, when W is an electrophile, an amide or an ester bond formation, or the like, can be performed. In Scheme 2, R³ can be a chemical group that would be useful in forming a covalent bond with the PLM. In one embodiment, R³ is protected by a commonly known protecting group such that it does not interfere with the reaction of forming a covalent bond between PTC and LI.

Schemes 1 and 2 represents examples of means and positions of the covalent bond formation between PTC and LI but is not meant to be limiting examples. Further, in preparation of the protac molecules of the present disclosure, the covalent bond between PLM and LI can be formed first followed by bond formation between LI and PTC.

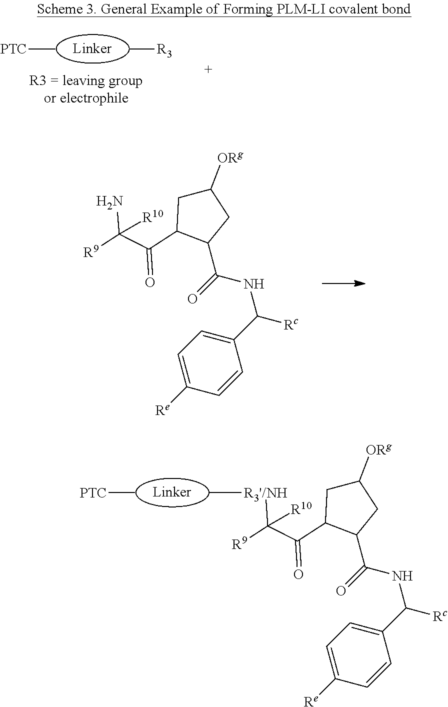

Scheme 3 demonstrates one way of forming a bond between the linker LI and PLM. The PLM shown in Scheme 3 is an example of a VHL. In Scheme 3, leaving group can be any group commonly known to person skilled in the art, including but not limited to halogen and sulfonates (e.g., tosylate, mesylate). In Scheme 3, electrophile can be any group commonly known to person skilled in the art, including but not limited to carboxylic acid. In Scheme 3, primary amine group is acting as a nucleophile to form a bond between the linker and the PLM. In one embodiment, in Scheme 3, PTC-Linker-R³ can be examples from Schemes 1 and 2.

### Representative Synthesis of Compounds of the Invention

### Example 46: Synthesis of (2S,4R)-1-((S)-2-(2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide

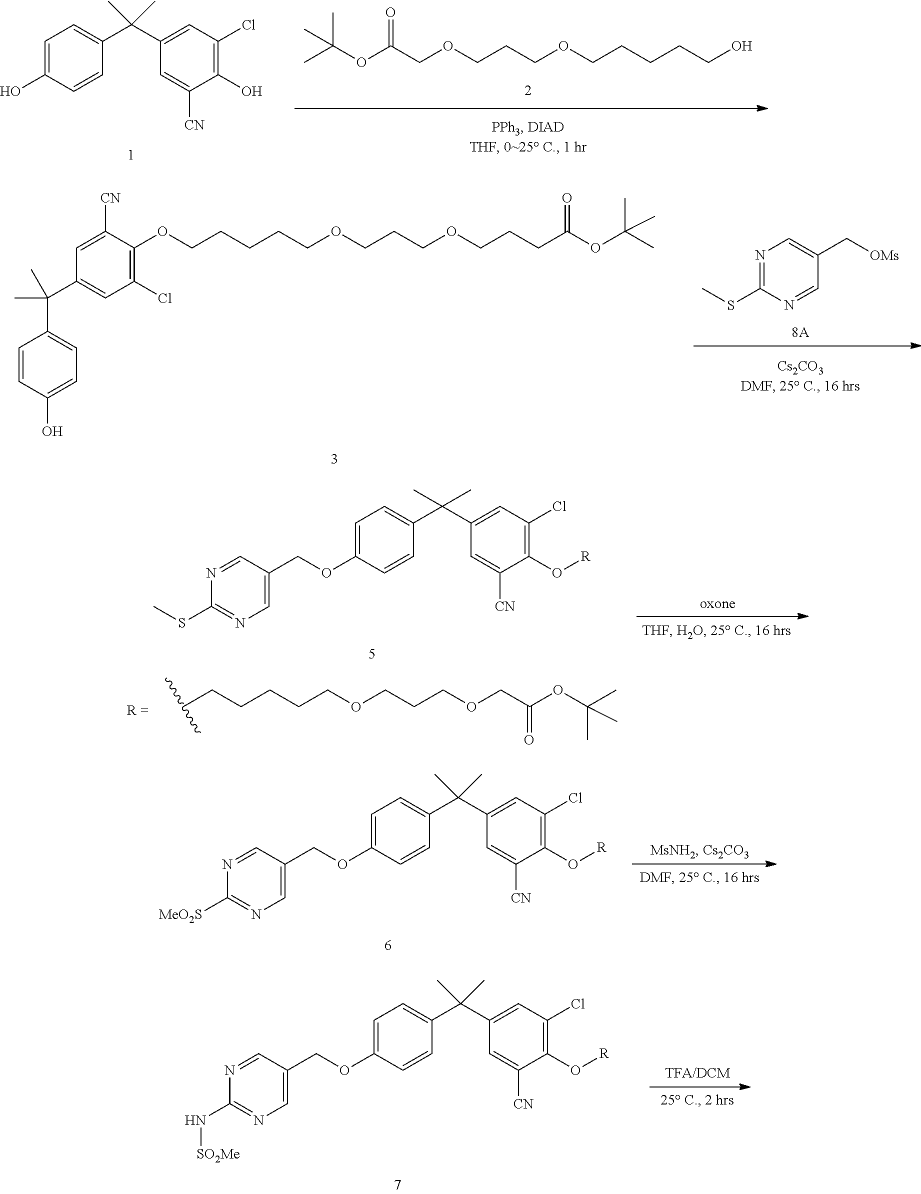

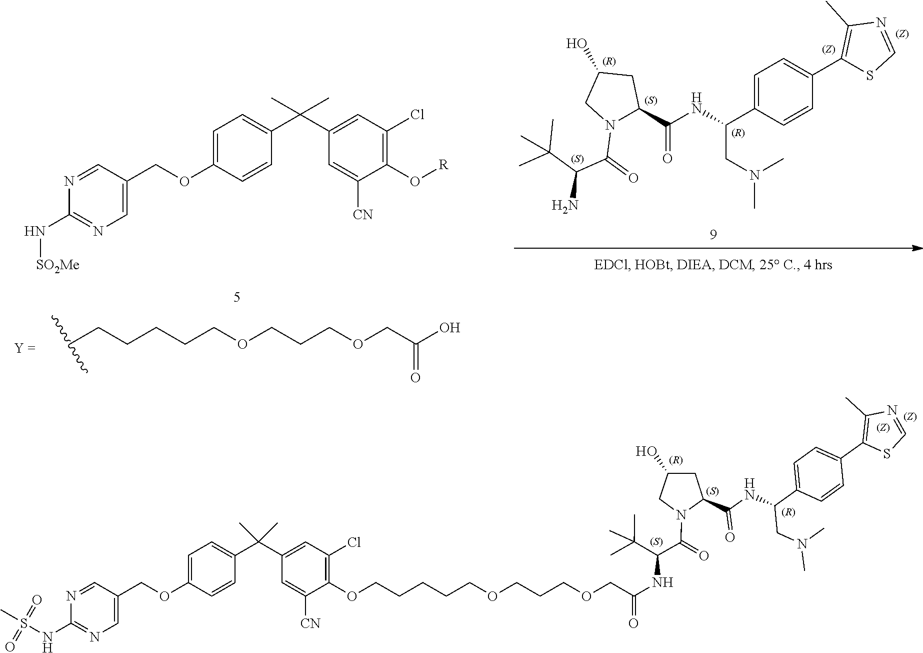

tert-Butyl 2-(3-((5-(2-chloro-6-cyano-4-(2-(4-hydroxyphenyl)propan-2-yl)phenoxy)pentyl) oxy)propoxy)acetate (3): To a solution of tert-butyl 2-(3-((5-hydroxypentyl)oxy)propoxy) acetate (2.0 g, 7.3 mmol) and 3-chloro-2-hydroxy-5-(2-(4-hydroxyphenyl)propan-2-yl) benzonitrile (2.1 g, 7.3 mmol) in THF (30 mL) was added PPh₃ (2.9 g, 10.9 mmol) and DIAD (2.1 mL, 10.9 mmol) at 0° C. under N2 atmosphere. The mixture was stirred at 25° C. for 1 h. TLC showed the reaction was completed. The resulting mixture was poured into water (100 mL), extracted with EtOAc (20 mL×3). The combined organic layers were washed with brine (50 mL), dried over Na₂SO₄, filtered and concentrated under reduced pressure. The residue was purified by silica gel column chromatography to give tert-butyl 2-(3-((5-(2-chloro-6-cyano-4-(2-(4-hydroxyphenyl)propan-2-yl)phenoxy)pentyl)oxy) propoxy)acetate (2.2 g, yield: 55.7%) as yellow oil. ¹H NMR (400 MHz, CDCl₃) δ=7.41 (d, J=2.4 Hz, 1H), 7.32 (d, J=2.4 Hz, 1H), 7.08-7.00 (m, 2H), 6.82-6.74 (m, 2H), 4.18 (t, J=6.4 Hz, 2H), 3.96 (s, 2H), 3.61 (t, J=6.4 Hz, 2H), 3.53 (t, J=6.4 Hz, 2H), 3.45 (t, J=6.4 Hz, 2H), 1.88 (sxt, J=6.4 Hz, 4H), 1.68-1.56 (m, 10H), 1.48 (s, 9H).

tert-Butyl 2-(3-((5-(2-chloro-6-cyano-4-(2-(4-((2-(methylthio)pyrimidin-5-yl)methoxy) phenyl)propan-2-yl)phenoxy)pentyl)oxy)propoxy)acetate (5): To a solution of tert-butyl 2-(3-((5-(2-chloro-6-cyano-4-(2-(4-hydroxyphenyl) propan-2-yl)phenoxy) pentyl)oxy)propoxy) acetate (3.0 g, 5.5 mmol) and Cs₂CO₃ (3.2 g, 9.8 mmol) in DMF (30 mL) was added (2-(methylthio)pyrimidin-5-yl)methyl methanesulfonate (1.5 g, 6.6 mmol) at 25° C. The mixture was stirred at the same temperature for 16 hrs. TLC showed the reaction was completed. The mixture was poured into water (50 mL), extracted with EtOAc (40 mL×2). The combined organic layers were washed with brine (50 mL×3), dried over Na₂SO₄, filtered and concentrated under reduced pressure. The residue was purified by MPLC to give tert-butyl 2-(3-((5-(2-chloro-6-cyano-4-(2-(4-((2-(methylthio)pyrimidin-5-yl)methoxy)phenyl)propan-2-yl)phenoxy)pentyl) oxy)propoxy)acetate (1.5 g, yield: 39.9%) as yellow oil. ¹H NMR (400 MHz, CDCl₃) δ=8.61 (s, 2H), 7.42 (d, J=2.2 Hz, 1H), 7.30 (d, J=2.2 Hz, 1H), 7.13 (d, J=8.8 Hz, 2H), 6.90 (d, J=8.8 Hz, 2H), 4.99 (s, 2H), 4.18 (t, J=6.4 Hz, 2H), 3.96 (s, 2H), 3.61 (t, J=6.4 Hz, 2H), 3.53 (t, J=6.4 Hz, 2H), 3.46 (t, J=6.4 Hz, 2H), 2.59 (s, 3H), 1.94-1.84 (m, 4H), 1.73-1.54 (m, 10H), 1.48 (s, 9H).

tert-Butyl 2-(3-((5-(2-chloro-6-cyano-4-(2-(4-((2-(methylsulfonyl)pyrimidin-5-yl)methoxy) phenyl)propan-2-yl)phenoxy)pentyl)oxy)propoxy)acetate (6): To a solution of tert-butyl 2-(3-((5-(2-chloro-6-cyano-4-(2-(4-((2-(methylthio)pyrimidin-5-yl) methoxy)phenyl)propan-2-yl)phenoxy)pentyl)oxy)propoxy)acetate (1.5 g, 2.2 mmol) in THF (10 mL) and water (10 mL) was added oxone (4.0 g, 6.6 mmol). The mixture was stirred at 25° C. for 16 hrs. TLC showed the reaction was completed. The reaction mixture was poured into saturated aqueous Na₂SO₃ (40 mL), extracted with EtOAc (30 mL×2), the combined organic layers were washed with brine (40 mL), dried over Na₂SO₄, filtered and concentrated under reduced pressure. The residue was purified by prep-TLC to give tert-butyl 2-(3-((5-(2-chloro-6-cyano-4-(2-(4-((2-(methylsulfonyl)pyrimidin-5-yl)methoxy)phenyl)propan-2-yl) phenoxy)pentyl)oxy)propoxy) acetate (1.5 g, yield: 95.5%) as white solid. ¹H NMR (400 MHz, CDCl₃) δ=9.02 (s, 2H), 7.42 (d, J=2.2 Hz, 1H), 7.29 (d, J=2.2 Hz, 1H), 7.16 (d, J=8.6 Hz, 2H), 6.92 (d, J=8.6 Hz, 2H), 5.21 (s, 2H), 4.22-4.15 (m, 2H), 3.96 (s, 2H), 3.61 (t, J=6.4 Hz, 2H), 3.53 (t, J=6.4 Hz, 2H), 3.45 (t, J=6.4 Hz, 2H), 3.39 (s, 3H), 1.94-1.84 (m, 4H), 1.71-1.57 (m, 10H), 1.48 (s, 9H).

tert-Butyl 2-(3-((5-(2-chloro-6-cyano-4-(2-(4-((2-(methylsulfonamido)pyrimidin-5-yl) methoxy)phenyl)propan-2-yl)phenoxy)pentyl)oxy)propoxy)acetate (7): To a solution of tert-butyl 2-(3-((5-(2-chloro-6-cyano-4-(2-(4-((2-(methylsulfonyl)pyrimidin-5-yl)methoxy) phenyl)propan-2-yl)phenoxy)pentyl)oxy)propoxy)acetate (1.5 g, 2.1 mmol) in MeCN (20 mL) was added MsNH₂ (597 mg, 6.3 mmol) and Cs₂CO₃ (2.1 g, 6.3 mmol). The mixture was stirred at 25° C. for 16 hrs. LCMS showed the reaction was completed. The residue was poured into H₂O (40 mL), extracted with EtOAc (20 mL×2), the combined organic layers were washed with brine (50 mL), dried over Na₂SO₄, filtered and concentrated under reduced pressure to give tert-butyl 2-(3-((5-(2-chloro-6-cyano-4-(2-(4-((2-(methylsulfonamido) pyrimidin-5-yl)methoxy)phenyl)propan-2-yl)phenoxy)pentyl)oxy)propoxy) acetate (1.10 g, yield: 71.8%) as yellow solid. ¹H NMR (400 MHz, CDCl₃) δ=8.70 (br s, 2H), 7.42 (d, J=2.4 Hz, 1H), 7.30 (d, J=2.4 Hz, 1H), 7.13 (br d, J=8.6 Hz, 2H), 6.89 (br d, J=8.6 Hz, 2H), 4.98 (br s, 2H), 4.18 (t, J=6.6 Hz, 2H), 3.96 (s, 2H), 3.61 (t, J=6.4 Hz, 2H), 3.53 (t, J=6.4 Hz, 2H), 3.48-3.37 (m, 5H), 3.11 (s, 1H), 1.93-1.86 (m, 4H), 1.71-1.58 (m, 10H), 1.48 (s, 9H).

2-(3-((5-(2-Chloro-6-cyano-4-(2-(4-((2-(methylsulfonamido)pyrimidin-5-yl)methoxy)phenyl)propan-2-yl)phenoxy)pentyl)oxy)propoxy)acetic acid (8): To a solution of tert-butyl 2-(3-((5-(2-chloro-6-cyano-4-(2-(4-((2-(methylsulfonamido)pyrimidin-5-yl) methoxy)phenyl) propan-2-yl)phenoxy)pentyl)oxy)propoxy) acetate (1.0 g, 1.5 mmol) in DCM (10 mL) was added TFA (2 mL) and the mixture was stirred at 25° C. for 4 hrs. LCMS showed the reaction was completed. The residue was concentrated under reduced pressure to give 2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy)acetic acid (800 mg, yield: 87.3%) as yellow oil. ¹H NMR (400 MHz, DMSO-d₆) δ=8.72 (s, 2H), 7.60 (d, J=2.3 Hz, 1H), 7.54 (d, J=2.3 Hz, 1H), 7.19 (d, J=8.9 Hz, 2H), 6.97 (br d, J=8.9 Hz, 2H), 5.05 (s, 2H), 4.12 (t, J=6.4 Hz, 2H), 3.96 (s, 2H), 3.48 (t, J=6.4 Hz, 2H), 3.41 (t, J=6.4 Hz, 2H), 3.38-3.32 (m, 5H), 2.91 (s, 1H), 1.81-1.68 (m, 4H), 1.63 (s, 6H), 1.57-1.49 (m, 4H).

(2S,4R)-1-((S)-2-(2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide: To a solution of 2-(3-((5-(2-chloro-6-cyano-4-(2-(4-((2-(methylsulfonamido)pyrimidin-5-yl)methoxy)phenyl)propan-2-yl)phenoxy)pentyl) oxy)propoxy)acetic acid (200 mg, 0.3 mmol), (2S,4R)-1-((S)-2-amino-3,3-dimethylbutanoyl)-N—((R)-2-(dimethylamino)-1-(4-(4-methylthiazol-5-yl)phenyl)ethyl)-4-hydroxypyrrolidine-2-carboxamide (144 mg, 0.3 mmol), EDCI (68 mg, 0.4 mmol) and HOBT (54 mg, 0.4 mmol) in DCM (2 mL) was added DIEA (0.1 mL, 0.6 mmol) and the mixture was stirred at 25° C. for 16 hrs. LCMS showed the reaction was completed. The mixture was poured into H₂O (8 mL), extracted with DCM (4 mL×2), and the combined organic layers were washed with brine (10 mL), dried over Na₂SO₄, filtered and concentrated under reduced pressure. The residue was purified by prep-HPLC (FA) to give (2S,4R)-1-((S)-2-(2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide (19.8 mg, yield: 5.84%) as white solid. ¹H NMR (400 MHz, DMSO-d₆) δ=9.04 (s, 1H), 8.73 (s, 2H), 8.38 (br d, J=7.4 Hz, 1H), 7.66 (d, J=2.4 Hz, 1H), 7.60 (d, J=2.4 Hz, 1H), 7.50-7.39 (m, 5H), 7.24 (d, J=8.8 Hz, 2H), 7.02 (d, J=8.8 Hz, 2H), 5.16 (br s, 1H), 5.09 (s, 2H), 4.94 (q, J=7.6 Hz, 1H), 4.60 (d, J=9.4 Hz, 1H), 4.53 (t, J=8.0 Hz, 1H), 4.33 (br s, 1H), 4.18 (t, J=6.4 Hz, 2H), 3.97 (s, 2H), 3.68-3.56 (m, 5H), 3.50 (brt, J=6.4 Hz, 3H), 3.47-3.40 (m, 7H), 2.66-2.59 (m, 4H), 2.52 (s, 3H), 2.25 (s, 6H), 2.15-2.05 (m, 1H), 1.90-1.76 (m, 6H), 1.68 (s, 6H), 1.63-1.53 (m, 4H), 0.99 (s, 9H). LCMS (220 nm): 95.0%, Exact Mass: 1143.1; found: 1144.3/1146.4.

### Example 47: Synthesis of (2S,4R)-1-((S)-2-(2-((5-(2-chloro-6-cyano-4-(2-(4-((2-(methylsulfonamido)pyrimidin-5-yl)methoxy)phenyl)propan-2-yl)phenoxy)pentyl)oxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide

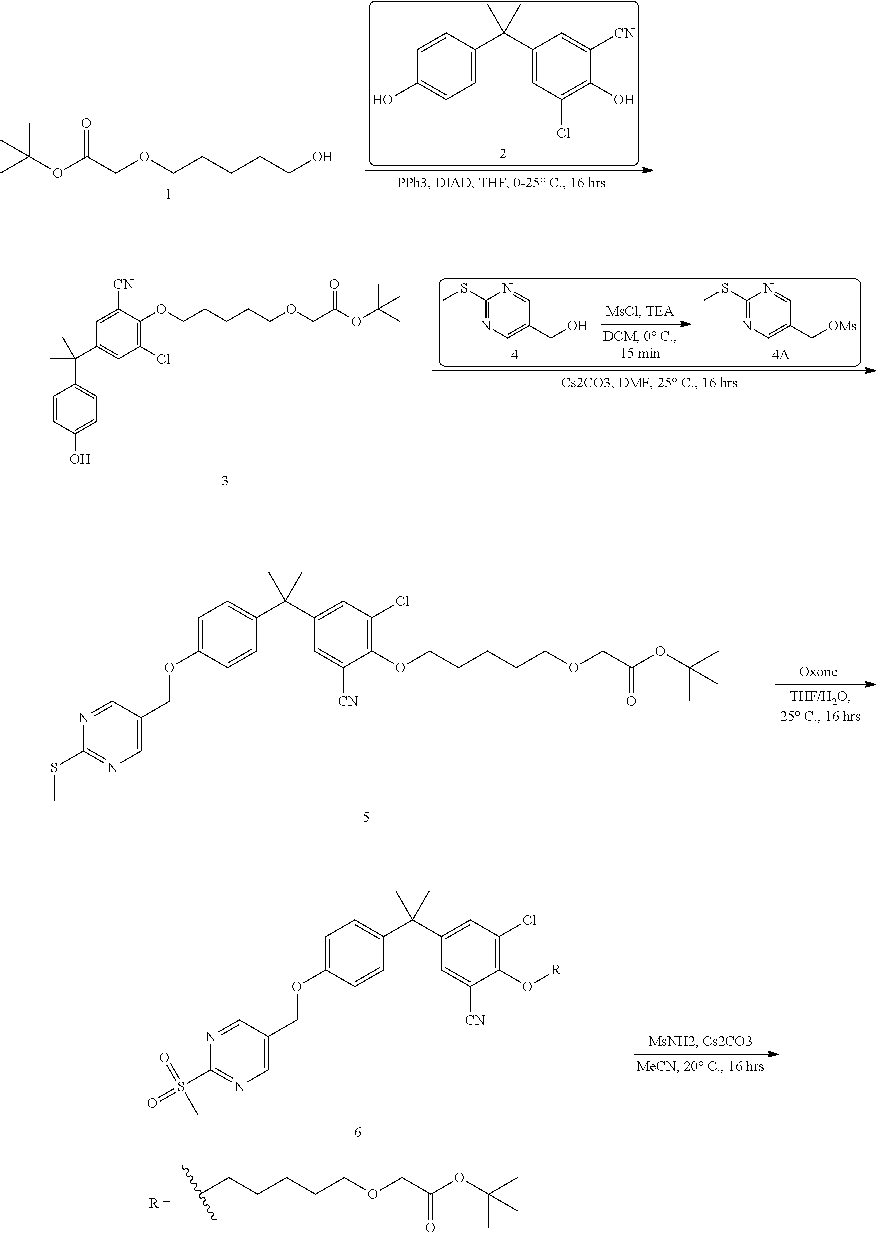

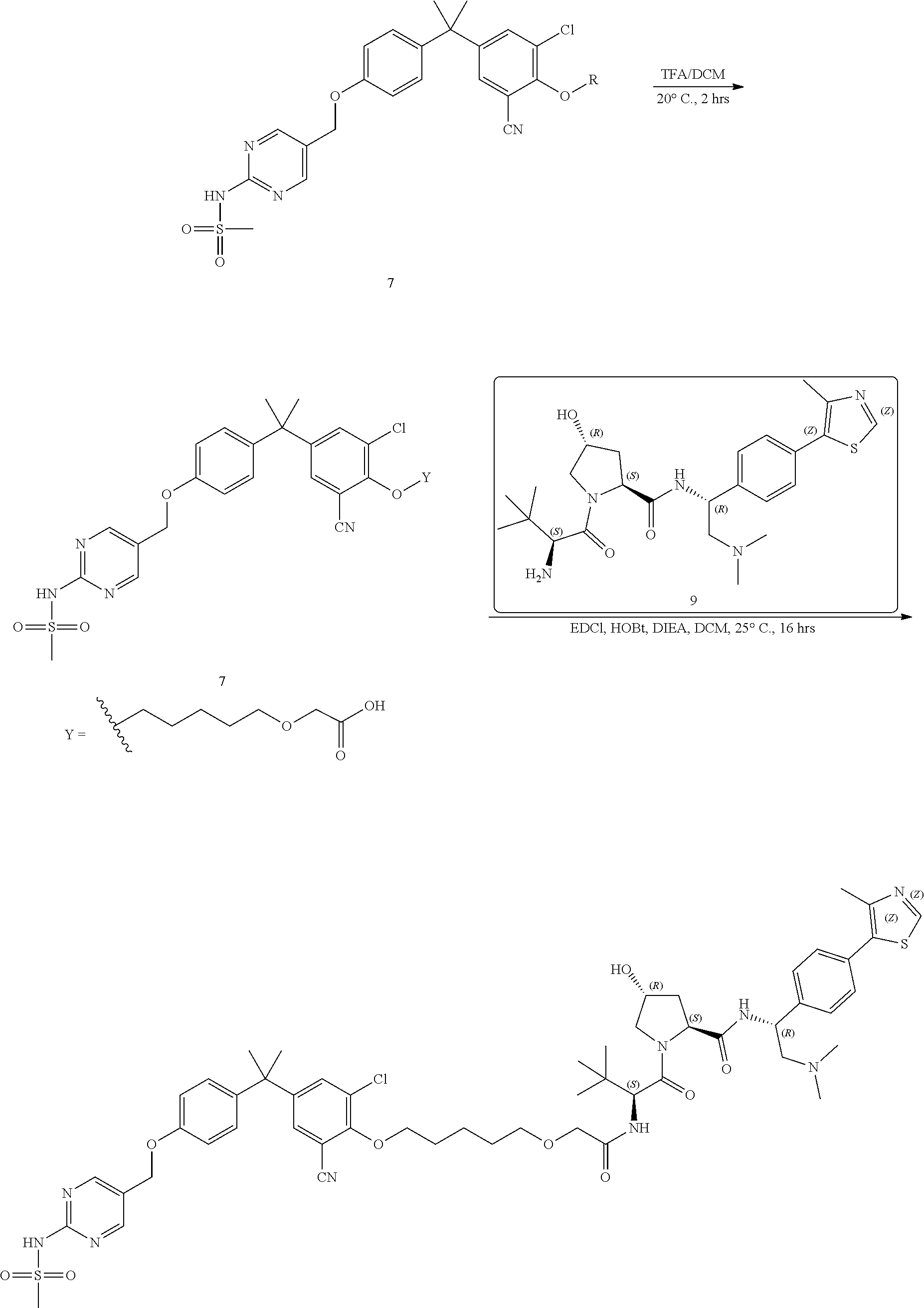

tert-Butyl 2-((5-(2-chloro-6-cyano-4-(2-(4-hydroxyphenyl)propan-2-yl)phenoxy)pentyl) oxy)acetate (3): To a solution of tert-butyl 2-((5-hydroxypentyl)oxy)acetate (1) (1.50 g, 6.87 mmol) and 3-chloro-2-hydroxy-5-(2-(4-hydroxyphenyl)propan-2-yl)benzonitrile (2) (1.98 g, 6.87 mmol) in THF (20 mL) was added PPh₃ (2.71 g, 10.3 mmol) and DIAD (2.03 mL, 10.3 mmol) at 0° C. under N2 atmosphere. The mixture was stirred at 25° C. for 16 hrs. TLC showed the starting material was consumed. The reaction mixture was poured into water (10 mL) and extracted with EtOAc (10 mL×3). The combined organic layers were washed with brine (10 mL×3), filtered and concentrated under reduced pressure. The residue was purified by silica-gel column chromatography to give tert-butyl 2-((5-(2-chloro-6-cyano-4-(2-(4-hydroxyphenyl)propan-2-yl)phenoxy)pentyl) oxy)acetate (3) (80.0% purity, 450 mg, yield: 28.6%) as yellow oil. ¹H NMR (400 MHz, CDCl₃) δ ppm 7.45 (d, J=2.4 Hz, 1H) 7.31 (d, J=2.4 Hz, 1H) 7.04 (d, J=8.8 Hz, 2H) 6.78 (d, J=8.4 Hz, 2H) 5.42 (s, 1H) 4.18 (t, J=6.4 Hz, 2H) 3.96 (s, 2H) 3.55 (t, J=6.4 Hz, 2H) 3.46 (m, 1H) 1.86-1.93 (m, 2H) 1.68-1.73 (m, 2H) 1.63 (s, 6H) 1.48 (s, 9H).

(2-(methylthio) pyrimidin-5-yl) methyl methanesulfonate (4A): To a solution of (2-methylsulfanylpyrimidin-5-yl)methanol (500 mg, 3.2 mmol) and TEA (0.669 mL, 0.48 mmol) in DCM (4 mL) was added MsCl (440 mg, 3.84 mmol) dropwise at 0° C. The mixture was stirred at the same temperature for 15 min. TLC showed the reaction was completed and the resulting mixture was quenched with water (10 mL) and extracted with DCM (10 mL×3). The combined organic layers were washed with brine (10 mL), dried over Na₂SO₄, filtered and concentrated under reduced pressure to give (2-(methylthio) pyrimidin-5-yl) methyl methanesulfonate (4A) (0.6 g, yield: 80.0%) as yellow solid. ¹H NMR (400 MHz, CDCl₃) δ ppm 9.46 (d, J=2.4 Hz, 1H) 8.70 (d, J=2.4 Hz, 1H) 5.93 (s, 2H) 2.83 (s, 3H) 2.24 (s, 3H).

tert-Butyl 2-((5-(2-chloro-6-cyano-4-(2-(4-((2-(methylthio)pyrimidin-5-yl)methoxy)phenyl)propan-2-yl)phenoxy)pentyl)oxy)acetate (5): To a solution of tert-butyl 2-((5-(2-chloro-6-cyano-4-(2-(4-hydroxyphenyl)propan-2-yl)phenoxy)pentyl)oxy)acetate (3) (1.25 g, 2.56 mmol) and Cs₂CO₃ (2.50 g, 7.68 mmol) in DMF (15 mL) was added (2-(methylthio) pyrimidin-5-yl) methyl methanesulfonate (4A) (600 mg, 2.56 mmol). The mixture was stirred at 25° C. for 16 hrs. LCMS showed the starting material was consumed. The solution was quenched with water (15 mL) and extracted with EtOAc (30 mL×3). The combined organic layers were washed with brine (15 mL×4), dried over Na₂SO₄, filtered and concentrated under reduced pressure. The crude was purified by silica gel column to give tert-butyl 2-((5-(2-chloro-6-cyano-4-(2-(4-((2-(methylthio)pyrimidin-5-yl)methoxy)phenyl) propan-2-yl)phenoxy)pentyl)oxy)acetate (5) (1.08 g, yield: 67.3%) as yellow oil. ¹H NMR (400 MHz, CDCl₃) δ ppm 8.60 (s, 2H) 7.42 (d, J=2.4 Hz, 1H) 7.30 (d, J=2.4 Hz, 1H) 7.13 (d, J=8.8 Hz, 2H) 6.90 (d, J=8.4 Hz, 2H) 4.99 (s, 2H) 4.19 (t, J=6.4 Hz, 2H) 3.56 (t, J=6.4 H.z, 2H) 2.59 (s, 3H) 1.87-1.94 (m, 2H) 1.65-1.74 (m, 2H) 1.63-1.64 (m, 8H) 1.49 (s, 9H).

tert-Butyl 2-((5-(2-chloro-6-cyano-4-(2-(4-((2-(methylsulfonyl) pyrimidin-5-yl)methoxy) phenyl)propan-2-yl)phenoxy)pentyl)oxy)acetate (6): To a solution of tert-butyl 2-((5-(2-chloro-6-cyano-4-(2-(4-((2-(methylthio)pyrimidin-5-yl)methoxy)phenyl) propan-2-yl)phenoxy)pentyl)oxy)acetate (5) (1.08 g, 1.72 mmol) in THF (10 mL) and water (10 mL) was added Oxone (2.65 g, 4.31 mmol). The mixture was stirred at 25° C. for 16 hrs. LCMS showed the reaction was completed. The reaction was quenched with water (10 mL) and extracted with EtOAc (10 mL×3), the combined organic layers were washed with brine (10 mL), dried over Na₂SO₄, filtered and concentrated under reduced pressure. The crude was purified by silica gel column to give tert-butyl 2-((5-(2-chloro-6-cyano-4-(2-(4-((2-(methylsulfonyl)pyrimidin-5-yl)methoxy)phenyl)propan-2-yl)phenoxy)pentyl)oxy)acetate (6) (0.410 g, yield: 36.1%) as colorless oil. ¹H NMR (400 MHz, CDCl₃) δ ppm 9.02 (s, 2H) 7.42 (d, J=2.4 Hz, 1H) 7.29 (d, J=2.4 Hz, 1H) 7.16 (d, J=8.8 Hz, 2H) 6.92 (d, J=9.2 Hz, 2H) 5.21 (s, 2H) 4.19 (t, J=6.4 Hz, 2H) 3.96 (s, 2H) 3.56 (t, J=6.0 Hz, 2H) 3.39 (s, 3H) 1.88-1.91 (m, 2H) 1.70-1.74 (m, 2H) 1.60-1.65 (m, 8H) 1.49 (s, 9H).

tert-Butyl 2-((5-(2-chloro-6-cyano-4-(2-(4-((2-(methylsulfonamido) pyrimidin-5-yl)methoxy) phenyl)propan-2-yl)phenoxy)pentyl)oxy)acetate (7): To a solution of tert-butyl 2-((5-(2-chloro-6-cyano-4-(2-(4-((2-(methylsulfonyl)pyrimidin-5-yl)methoxy) phenyl)propan-2-yl) phenoxy)pentyl)oxy)acetate (6) (190 mg, 2.83 mmol) in CH₃CN (3 mL) was added MsNH₂ (80.6 mg, 0.85 mmol) and Cs₂CO₃ (276 mg, 0.85 mmol) at 20° C. The mixture was stirred for 16 hrs at 20° C. LCMS showed the reaction was completed and the resulting mixture was quenched with H₂O (10 mL) and EtOAc (10 mL×2). The combined organic layers were washed with brine (10 mL), dried over Na₂SO₄, filtered and concentrated under reduced pressure to give tert-butyl 2-((5-(2-chloro-6-cyano-4-(2-(4-((2-(methylsulfonamido)pyrimidin-5-yl)methoxy)phenyl)propan-2-yl)phenoxy)pentyl)oxy)acetate (7) (160 mg, yield: 82.4%) as yellow oil. ¹H NMR (400 MHz, CDCl₃) δ ppm 8.68 (s, 2H) 7.42 (d, J=2.4 Hz, 1H) 7.29 (d, J=2.4 Hz, 1H) 7.14 (d, J=8.8 Hz, 2H) 6.90 (d, J=8.8 Hz, 2H) 5.02 (s, 2H) 4.19 (t, J=6.4 Hz, 2H) 3.96 (s, 2H) 3.56 (t, J=6.4 Hz, 2H) 3.49 (s, 3H) 1.88-1.92 (m, 2H) 1.70-1.72 (m, 2H) 1.69 (s, 6H) 1.60-1.65 (m, 2H) 1.49 (s, 9H).

2-((5-(2-chloro-6-cyano-4-(2-(4-((2-(methylsulfonamido)pyrimidin-5-yl)methoxy)phenyl)propan-2-yl)phenoxy)pentyl)oxy)acetic acid (8): A mixture of tert-butyl 2-((5-(2-chloro-6-cyano-4-(2-(4-((2-(methylsulfonamido)pyrimidin-5-yl)methoxy) phenyl)propan-2-yl)phenoxy) pentyl)oxy)acetate (7) (150 mg, 0.23 mmol) in DCM (3 mL) was added TFA (0.50 mL) and stirred at 20° C. for 2 hrs. TLC showed most of the starting materiel consumed and ˜50% of desired product. The mixture was concentrated under reduced pressure to give 2-((5-(2-chloro-6-cyano-4-(2-(4-((2-(methylsulfonamido)pyrimidin-5-yl)methoxy)phenyl) propan-2-yl)phenoxy) pentyl) oxy)acetic acid (8) (140 mg, yield: 99%) as colorless oil. ¹H NMR (400 MHz, CDCl₃) δ ppm 8.71 (s, 2H) 7.42 (d, J=2.4 Hz, 1H) 7.31 (d, J=2.4 Hz, 1H) 7.13 (d, J=8.8 Hz, 2H) 6.90 (d, J=8.8 Hz, 2H) 5.03 (s, 2H) 4.20 (t, J=6.4 Hz, 2H) 4.11 (s, 2H) 3.63 (t, J=6.0 Hz, 2H) 3.48 (s, 3H) 1.88-1.93 (m, 2H) 1.67-1.75 (m, 4H) 1.65 (s, 6H).

(2S, 4R)-1-((S)-2-(2-((5-(2-chloro-6-cyano-4-(2-(4-((2-(methylsulfonamido)pyrimidin-5-yl)methoxy)phenyl)propan-2-yl)phenoxy)pentyl)oxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide: To a solution of 2-((5-(2-chloro-6-cyano-4-(2-(4-((2-(methylsulfonamido) pyrimidin-5-yl)methoxy)phenyl) propan-2-yl)phenoxy)pentyl) oxy)acetic acid (8) (100 mg, 0.16 mmol), (2S,4R)-1-((S)-2-amino-3,3-dimethylbutanoyl)-N—((R)-2-(dimethylamino)-1-(4-(4-methylthiazol-5-yl)phenyl)ethyl)-4-hydroxypyrrolidine-2-carboxamide (9) (114 mg, 0.19 mmol), EDC HCl (31.1 mg, 0.16 mmol) and HOBT (24.8 mg, 0.16 mmol) in DCM (2 mL) was added DIEA (0.139 mL, 0.81 mmol) and the mixture was stirred at 25° C. for 16 hrs. LCMS showed the reaction was completed. The mixture was poured into H₂O (5 mL) and extracted with DCM (5 mL×3), and the combined organic layers were washed with brine (5 mL×2), dried over Na₂SO₄, filtered and concentrated in vacuum to give the crude. The crude was purified by p-HPLC (FA) to give (2S, 4R)-1-((S)-2-(2-((5-(2-chloro-6-cyano-4-(2-(4-((2-(methylsulfonamido)pyrimidin-5-yl) methoxy)phenyl)propan-2-yl) phenoxy)pentyl)oxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide (57.6 mg, yield: 29.9%) as white solid. ¹H NMR (400 MHz, CDCl₃) δ ppm 9.53 (d, J=9.6 Hz, 1H) 8.87 (s, 1H) 8.72 (s, 2H) 7.43-7.51 (m, 5H) 7.29 (d, J=2.0 Hz, 1H) 7.13 (d, J=8.8 Hz, 2H) 6.89 (d, J=8.8 Hz, 2H) 5.56-5.61 (m, 1H) 5.02 (s, 2H) 4.51-4.68 (m, 4H) 4.17 (t, J=6.4 Hz, 2H) 3.92-4.03 (m, 4H) 3.55 (d, J=6.4 Hz, 1H) 3.46-3.53 (m, 2H) 3.13 (s, 3H) 2.99-3.02 (m, 1H) 2.98 (s, 6H) 2.53 (s, 3H) 2.27-2.29 (m, 1H) 1.86-1.97 (m, 3H) 1.69-1.74 (m, 2H) 1.65 (s, 6H) 1.04 (s, 9H) LCMS (220 nm): 95.36%, Exact Mass: 1085.4, Founded: 1086.4.

### Example 48: Synthesis of (2S,4R)-1-((S)-2-(2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy)acetamido)-3,3-dimethylbutanoyl)-4-hydroxy-N-(4-(4-methylthiazol-5-yl)benzyl)pyrrolidine-2-carbox amide

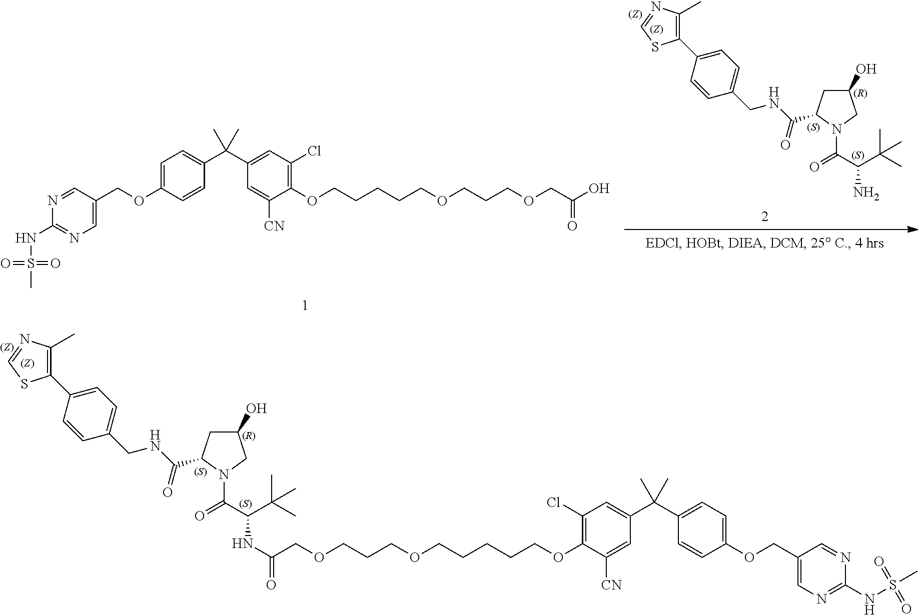

To a solution of 2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy) acetic acid (200 mg, 0.3 mmol), (2S,4R)-1-((S)-2-amino-3,3-dimethylbutanoyl)-4-hydroxy-N-(4-(4-methylthiazol-5-yl)benzyl)pyrrolidine-2-carboxamide (144 mg, 0.3 mmol), EDCI (68 mg, 0.4 mmol) and HOBT (55 mg, 0.4 mmol) in DCM (2 mL) was added DIEA (0.1 mL, 0.6 mmol) and the mixture was stirred at 25° C. for 16 hrs. LCMS showed the reaction was completed. The mixture was poured into H₂O (8 mL), extracted with DCM (4 mL×2), and the combined organic layers were washed with brine (10 mL), dried over Na₂SO₄, filtered and concentrated under reduced pressure. The crude was purified by prep-HPLC (FA) to give (2S,4R)-1-((S)-2-(2-(3-((5-(2-chloro-6-cyano-4-(2-(4-((2-(methylsulfonamido)pyrimidin-5-yl)methoxy)phenyl)propan-2-yl)phenoxy)pentyl)oxy)propoxy)acetamido)-3,3-dimethyl butanoyl)-4-hydroxy-N-(4-(4-methylthiazol-5-yl)benzyl)pyrrolidine-2-carbox-amide (13.0 mg, yield: 4.03%) as white solid. ¹H NMR (400 MHz, DMSO-d₆) δ=8.99-8.94 (m, 1H), 8.67 (s, 2H), 8.60 (br t, J=5.8 Hz, 1H), 7.60 (d, J=2.2 Hz, 1H), 7.54 (d, J=2.2 Hz, 1H), 7.45-7.31 (m, 5H), 7.18 (d, J=8.8 Hz, 2H), 6.97 (d, J=8.8 Hz, 2H), 5.15 (br s, 1H), 5.03 (s, 2H), 4.56 (d, J=9.6 Hz, 1H), 4.48-4.32 (m, 3H), 4.30-4.22 (m, 1H), 4.10 (t, J=6.4 Hz, 2H), 3.92 (s, 2H), 3.69-3.58 (m, 2H), 3.54 (br t, J=6.4 Hz, 2H), 3.45 (br t, J=6.4 Hz, 2H), 3.37 (br t, J=6.2 Hz, 2H), 3.31 (br s, 6H), 2.46-2.43 (m, 3H), 2.14-2.02 (m, 1H), 1.97-1.85 (m, 1H), 1.82-1.68 (m, 4H), 1.63 (s, 6H), 1.57-1.44 (m, 4H), 1.02-0.85 (m, 9H). LCMS (220 nm): 96.2%, Exact Mass: 1086.4; found: 1087.1/1089.2.

### Example 49: Synthesis of (2S,4R)-1-((S)-2-(2-(3-((5-(2-chloro-6-cyano-4-(2-(4-((2-(methylsulfonamido)pyrimidin-4-yl)methoxy)phenyl)propan-2-yl)phenoxy)pentyl)oxy)propoxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide

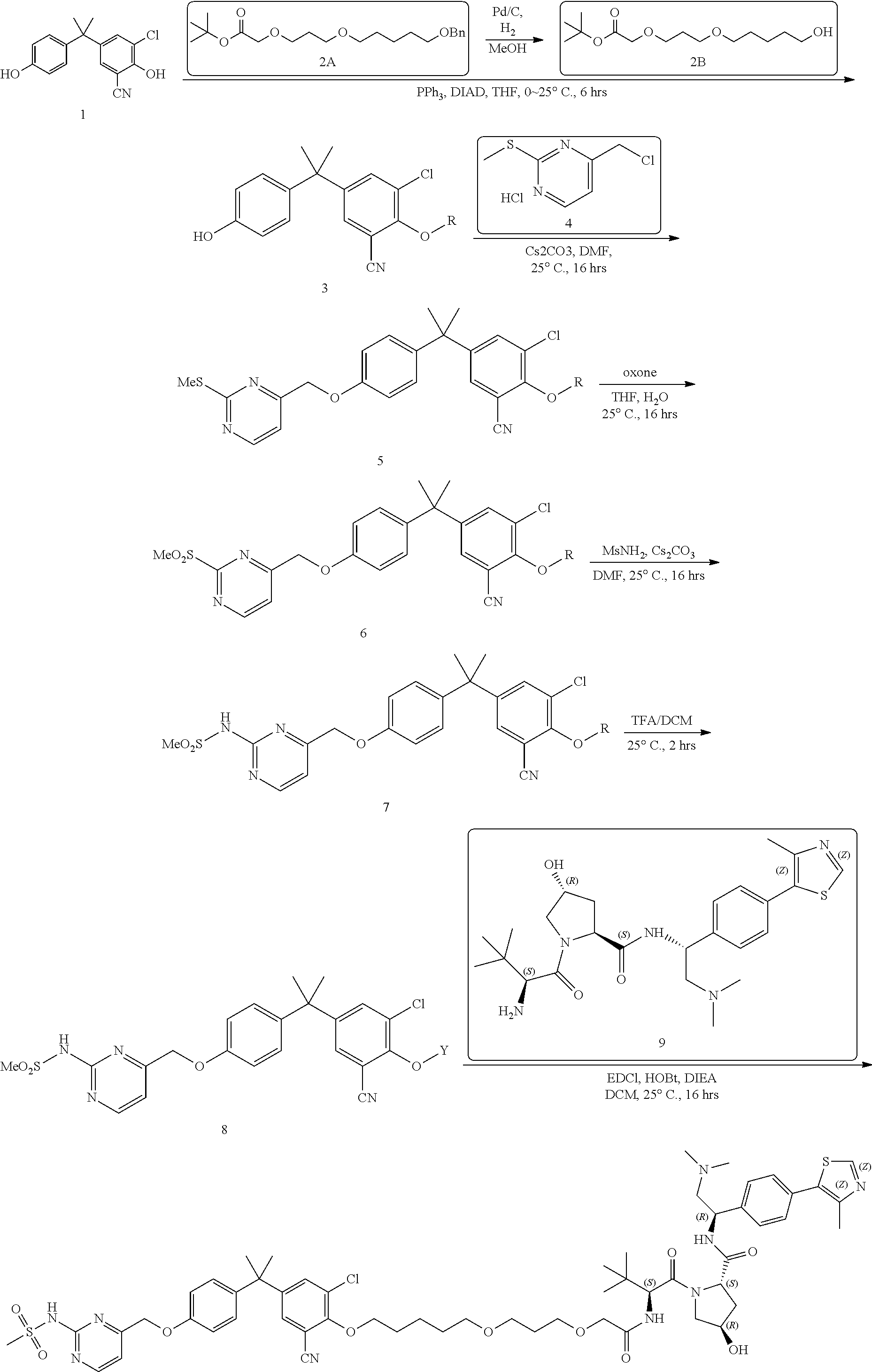

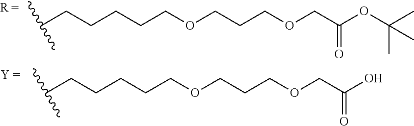

tert-Butyl 2-(3-(5-hydroxypentoxy)propoxy)acetate (21B): To a solution of tert-butyl 2-(3-(5-benzyloxypentoxy)propoxy)acetate (2A) (3.60 g, 11.7 mmol) in MeOH (108 mL) was added Pd/C (1000 purity, 1.50 g, 12.1 mmol) at 20° C. The mixture was stirred at 40° C. for 16 hrs under H₂ balloon (˜15 psi). LCMS showed the reaction was completed. The mixture was filtered by Celite, the filtrate was concentrated under reduced pressure to give tert-butyl 2-(3-(5-hydroxypentoxy)propoxy)acetate (21B) (3.20 g, yield: 86.7% o) as white oil. ¹H NMR (400 MHz, CDCl₃) δ ppm 3.96 (s, 2H) 3.63-3.68 (m, 2H) 3.61 (t, J=6.4 Hz, 2H) 3.53 (t, J=6.4 Hz, 2H) 3.44 (t, J=6.4 Hz, 2H) 1.89 (quin, J=6.4 Hz, 2H) 1.55-1.65 (m, 5H) 1.49 (s, 9H) 1.43-1.46 (m, 1H).

tert-Butyl 2-(3-(5-(2-chloro-6-cyano-4-(1-(4-hydroxyphenyl)-1-methyl-ethyl)phenoxy) pentoxy)propoxy)acetatete (3): To a solution of tert-butyl 2-(3-(5-hydroxypentoxy) propoxy)acetate (2B) (2.0 g, 9.16 mmol) and 3-chloro-2-hydroxy-5-(1-(4-hydroxyphenyl)-1-methyl-ethyl)benzonitrile (1) (2.64 g, 9.16 mmol) in THF (20 mL) was added PPh₃ (3.62 g, 13.7 mmol) and DIAD (2.71 mL, 13.7 mmol) at 0° C. under N2 atmosphere. The mixture was stirred at 25° C. for 16 hrs under N2. TLC showed the reaction was completed. The solution was poured into water (40 mL), extracted with EtOAc (20 mL×3). The combined organic layers were washed with brine (40 mL), dried over Na₂SO₄, filtered and concentrated under reduced pressure. The residue was purified by silica-gel column chromatography (petroleum ether: ethyl acetate=6:1-2:1) to give tert-butyl 2-(3-(5-(2-chloro-6-cyano-4-(1-(4-hydroxyphenyl)-1-methyl-ethyl)phenoxy)pentoxy) propoxy)acetate (3) (5.5 g, yield: 95%) as yellow oil. ¹H NMR (400 MHz, CDCl₃) δ ppm 7.41 (d, J=2.0 Hz, 1H), 7.31 (d, J=2.0 Hz, 1H), 7.04 (d, J=8.8 Hz, 2H), 6.78 (d, J=8.4 Hz, 2H), 5.70 (s, 1H), 4.17 (t, J=6.4 Hz, 2H), 3.96 (s, 2H), 3.60 (t, J=6.4 Hz, 2H), 3.53 (t, J=6.4 Hz, 2H), 3.45 (t, J=6.4 Hz, 2H), 1.88 (sxt, J=6.4 Hz, 4H) 1.56-1.67 (m, 10H), 1.48 (s, 9H).

tert-butyl 2-(3-(5-(2-chloro-6-cyano-4-(1-methyl-1-(4-((2-methylsulfanylpyrimidin-5-yl) methoxy)phenyl)ethyl) phenoxy)pentoxy)propoxy)acetate (5): To a solution of tert-butyl 2-(3-(5-(2-chloro-6-cyano-4-(1-(4-hydroxyphenyl)-1-methyl-ethyl)phenoxy)pentoxy)propoxy) acetate (3) (2.0 g, 3.66 mmol) in DMF (20 mL) was added 5-(chloromethyl)-2-methylsulfanyl-pyrimidine hydrochloride (774 mg, 3.76 mmol) and Cs₂CO₃ (4.77 g, 14.6 mmol) at 20° C. under N2. The mixture was stirred at 20° C. for 16 hrs under N2. LCMS showed the starting material consumed. The reaction mixture was poured into water (20 mL), extracted with EtOAc (20 mL×3). The combined organic layers were washed with brine (10 mL×3), dried over Na₂SO₄, filtered and concentrated under reduced pressure. The residue was purified by silica gel column (petroleum ether:ethyl acetate=6:1-2:1) to give tert-butyl 2-(3-(5-(2-chloro-6-cyano-4-(1-methyl-1-(4-((2-methylsulfanylpyrimidin-5-yl)methoxy)phenyl)ethyl)phenoxy)pentoxy) propoxy)acetate (5) (1.28 g, yield: 46.0%) as colorless oil. ¹H NMR (400 MHz, CDCl₃) δ ppm 8.54 (d, J=4.2 Hz, 1H), 7.42 (d, J=2.4 Hz, 1H), 7.30 (d, J=2.0 Hz, 1H), 7.22 (d, J=4.2 Hz, 1H), 7.12 (d, J=8.8 Hz, 2H), 6.89 (d, J=8.8 Hz, 2H), 5.09 (s, 2H), 4.17 (t, J=6.8 Hz, 2H), 3.96 (s, 2H), 3.61 (t, J=6.4 Hz, 2H), 3.53 (t, J=6.4 Hz, 2H), 3.45 (t, J=6.4 Hz, 2H), 2.59 (s, 3H), 1.83-1.93 (m, 4H), 1.57-1.72 (m, 10H), 1.48 (s, 9H).

tert-Butyl 2-(3-(5-(2-chloro-6-cyano-4-(1-methyl-1-(4-((2-methylsulfanylpyrimidin-4-yl)methoxy)phenyl)ethyl)phenoxy)pentoxy)propoxy) acetate (6): To a solution of tert-butyl 2-(3-(5-(2-chloro-6-cyano-4-(1-methyl-1-(4-((2-methylsulfanylpyrimidin-4-yl)methoxy)phenyl) ethyl)phenoxy)pentoxy)propoxy)acetate (5) (1.28 g, 1.87 mmol) in THF (13 mL) and H₂O (13 mL) was added Oxone (3.45 g, 5.61 mmol) at 20° C. The mixture was stirred for 16 hrs at 20° C. under N₂. LCMS showed the starting material consumed. The reaction mixture was poured into water (20 mL), extracted with EtOAc (15 mL×3). The combined organic layers were washed with brine (20 mL), dried over Na₂SO₄, filtered and concentrated under reduced pressure to give tert-butyl 2-(3-(5-(2-chloro-6-cyano-4-(1-methyl-1-(4-((2-methylsulfanylpyrimidin-4-yl)methoxy)phenyl)ethyl) phenoxy)pentoxy)propoxy)acetate (6) (1.22 g, yield: 73.3%) as colorless oil. ¹H NMR (400 MHz, CDCl₃) δ ppm 8.94 (d, J=4.2 Hz, 1H), 7.86 (d, J=4.2 Hz, 1H), 7.43 (d, J=2.4 Hz, 1H), 7.29 (d, J=2.0 Hz, 1H), 7.15 (d, J=8.8 Hz, 2H), 6.91 (d, J=9.2 Hz, 2H), 5.30 (s, 2H), 4.18 (t, J=6.4 Hz, 2H), 3.96 (s, 2H), 3.61 (t, J=6.4 Hz, 1H), 3.53 (t, J=6.4 Hz, 2H), 3.45 (t, J=6.4 Hz, 2H), 3.40 (s, 3H), 1.83-1.93 (m, 4H), 1.57-1.69 (m, 10H), 1.48 (s, 9H).

tert-Butyl 2-(3-(5-(2-chloro-6-cyano-4-(1-(4-((2-(methanesulfonamido)pyrimidin-4-yl) methoxy)phenyl)-1-methyl-ethyl)phenoxy)pentoxy)propoxy)acetate (7): To a solution of tert-butyl 2-(3-(5-(2-chloro-6-cyano-4-(1-methyl-1-(4-((2-methylsulfonylpyrimidin-4-yl)methoxy)phenyl)ethyl)phenoxy)pentoxy)propoxy)acetate (6) (1.22 g, 1.70 mmol) in DMF (13 mL) was added Methanesulfonamide (567 mg, 5.96 mmol) and Cs₂CO₃ (1.94 g, 5.96 mmol) at 20° C. The mixture was stirred at 20° C. for 16 hrs. LCMS showed the reaction was completed. The solution was poured into water (20 mL), extracted with EtOAc (10 mL×3). The combined organic layers were washed with brine (20 mL×3), dried over Na₂SO₄, filtered and concentrated under reduced pressure to give tert-butyl 2-(3-(5-(2-chloro-6-cyano-4-(1-(4-((2-(methanesulfonamido)pyrimidin-4-yl)methoxy)phenyl)-1-methyl-ethyl)phenoxy)pentoxy) propoxy)acetate (7) (820 mg, yield: 65.8%) as yellow oil. ¹H NMR (400 MHz, CDCl₃) δ ppm 8.64 (d, J=4.2 Hz, 1H) 7.42 (d, J=2.4 Hz, 1H) 7.28-7.30 (m, 2H) 7.12 (d, J=8.8 Hz, 2H) 6.90 (d, J=8.8 Hz, 2H) 5.11 (s, 2H) 4.18 (t, J=6.4 Hz, 2H) 3.96 (s, 2H) 3.61 (t, J=6.4 Hz, 2H) 3.53 (t, J=6.4 Hz, 2H) 3.40-3.48 (m, 4H) 2.05 (s, 3H) 1.82-1.95 (m, 4H) 1.56-1.68 (m, 10H) 1.48 (s, 9H).

2-(3-(5-(2-chloro-6-cyano-4-(1-(4-((2-(methanesulfonamido)pyrimidin-4-yl)methoxy) phenyl)-1-methyl-ethyl)phenoxy)pentoxy) acetic acid (8): To a solution of tert-butyl 2-(3-(5-(2-chloro-6-cyano-4-(1-(4-((2-(methanesulfonamido)pyrimidin-4-yl)methoxy)phenyl)-1-methyl-ethyl)phenoxy)pentoxy)propoxy)acetate (7) (500 mg, 0.684 mmol) in DCM (5 mL) was added TFA (2.5 mL) at 20° C., and the mixture were stirred at 25° C. for 2 hrs. TLC showed the reaction was completed. The solution was concentrated under reduced pressure. The residue was dissolved with DCM (5 mL), and added water (2 mL). The aqueous layers were extracted with DCM (2 mL×3). The combined organic layers were washed with brine (5 mL), dried over Na₂SO₄, filtered and concentrated under reduced pressure to give 2-(3-(5-(2-chloro-6-cyano-4-(1-(4-((2-(methanesulfonamido)pyrimidin-4-yl)methoxy)phenyl)-1-methyl-ethyl)phenoxy) pentoxy) acetic acid (8) (350 mg, yield: 75.8%) as yellow oil. ¹H NMR (400 MHz, CDCl₃) δ ppm 8.62 (d, J=4.8 Hz, 1H), 7.42 (d, J=2.4 Hz, 1H), 7.28-7.30 (m, 2H), 7.12 (d, J=8.8 Hz, 2H), 6.89 (d, J=8.8 Hz, 2H), 5.11 (s, 2H), 4.19 (t, J=6.4 Hz, 2H), 4.09 (s, 2H), 3.70 (t, J=5.6 Hz, 2H), 3.62 (t, J=5.6 Hz, 2H), 3.51 (br t, J=6.8 Hz, 2H), 3.47 (s, 3H), 1.83-1.95 (m, 6H), 1.66 (m, 9H).

(2S,4R)-1-((S)-2-(2-(3-((5-(2-chloro-6-cyano-4-(2-(4-((2-(methylsulfonamido)pyrimidin-4-yl)methoxy)phenyl)propan-2-yl)phenoxy)pentyl)oxy)propoxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide: To a solution of 2-(3-(5-(2-chloro-6-cyano-4-(1-(4-((2-(methanesulfonamido)pyrimidin-4-yl)methoxy)phenyl)-1-methyl-ethyl) henoxy)pentoxy) propoxy)acetic acid (8) (100 mg, 0.148 mmol), (2S,4R)-1-((2S)-2-amino-3,3-dimethyl-butanoyl)-N-((1R)-2-(dimethylamino)-1-(4-(4-methylthiazol-5-yl)phenyl)ethyl)-4-hydroxy-pyrrolidine-2-carboxamide (72.2 mg, 0.148 mmol), EDC HCl (34.1 mg, 0.178 mmol) and HOBT (27.2 mg, 0.178 mmol) in DCM (1.5 mL) was added DIEA (0.0507 mL, 0.296 mmol) and the mixture was stirred at 25° C. for 16 hrs. LCMS showed the starting material consumed and the desired was observed. The mixture was poured into H₂O (3 mL), extracted with DCM (2 mL×2), and the combined organic layers were washed with brine (10 mL), dried over Na₂SO₄, filtered and concentrated under reduced pressure. The crude was purified by prep-HPLC (NH₄HCO₃) to give (2S,4R)-1-((S)-2-(2-(3-((5-(2-chloro-6-cyano-4-(2-(4-((2-(methylsulfonamido)pyrimidin-4-yl)methoxy)phenyl)propan-2-yl)phenoxy)pentyl)oxy)propoxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide (18.1 mg, yield: 10.1%) as yellow oil. ¹H NMR (400 MHz, CDCl₃) δ ppm 8.68 (s, 1H), 8.60 (d, J=5.2 Hz, 1H), 7.70 (br d, J=5.2 Hz, 1H), 7.42 (d, J=2.0 Hz, 1H), 7.39 (d, J=8.4 Hz, 2H), 7.34 (d, J=8.4 Hz, 2H), 7.30 (d, J=2.0 Hz, 1H), 7.25 (d, J=5.2 Hz, 1H), 7.22 (br d, J=8.4 Hz, 1H), 7.12 (d, J=8.8 Hz, 2H), 6.89 (d, J=8.8 Hz, 2H), 5.09 (s, 2H), 4.90 (dt, J=10.4, 4.8 Hz, 1H), 4.78 (t, J=8.0 Hz, 1H), 4.54 (d, J=8.4 Hz, 1H), 4.50 (br s, 1H), 4.11-4.22 (m, 3H), 3.96 (s, 2H), 3.62 (t, J=6.4 Hz, 3H), 3.52 (t, J=6.4 Hz, 2H), 3.39-3.49 (m, 5H), 2.59-2.70 (m, 1H), 2.53 (s, 3H), 2.36-2.49 (m, 2H), 2.25 (s, 6H), 2.06-2.19 (m, 1H), 1.80-1.95 (m, 4H), 1.50-1.73 (m, 11H), 1.09 (s, 9H) LCMS (220 nm): 95.4%, Exact Mass: 1143.5; found: 1144.4/1145.4.

### Example 50: Synthesis of (2S,4R)-1-((S)-2-(2-((5-(2-chloro-6-cyano-4-(2-(4-((2-(methylsulfonamido)pyrimidin-4-yl)methoxy)phenyl)propan-2-yl)phenoxy)pentyl)oxy) acetamido)-3,3-dimethylbutanoyl)-N—((R)-2-(dimethylamino)-1-(4-(4-methylthiazol-5-yl) phenyl)ethyl)-4-hydroxypyrrolidine-2-carboxamide

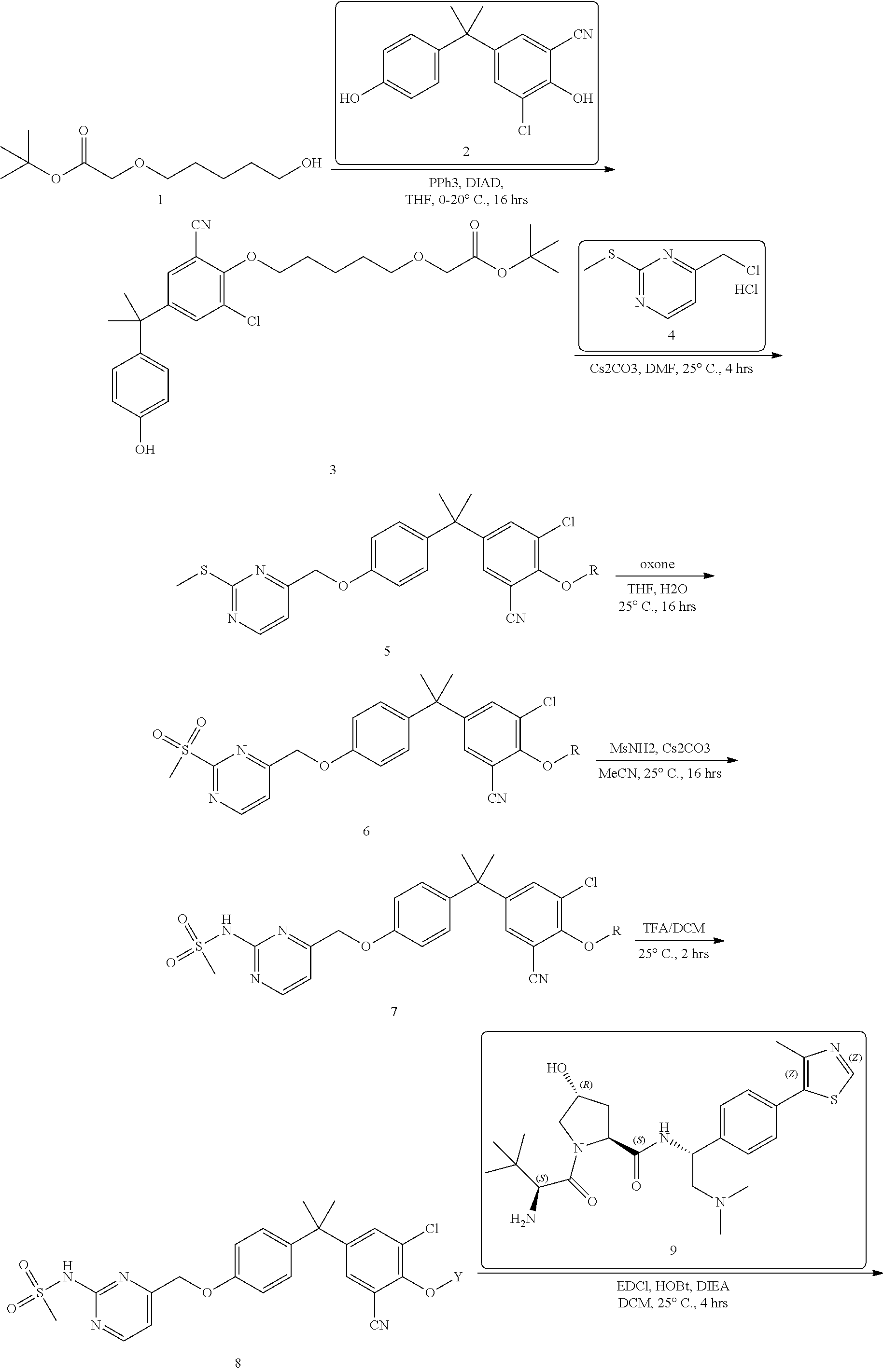

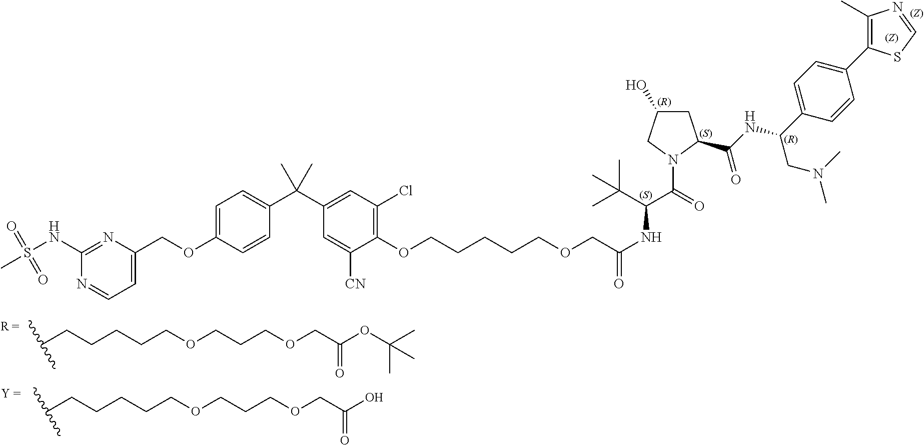

tert-Butyl 2-(5-(2-chloro-6-cyano-4-(1-(4-hydroxyphenyl)-1-methyl-ethyl)phenoxy) pentoxy)acetate (3): To a solution of tert-butyl 2-(5-hydroxypentoxy)acetate (1) (2.0 g, 9.16 mmol) and 3-chloro-2-hydroxy-5-(1-(4-hydroxyphenyl)-1-methyl-ethyl)benzonitrile (2) (2.64 g, 9.16 mmol) in THF (20 mL) was added PPh₃ (3.62 g, 13.7 mmol) and DIAD (2.71 mL, 13.7 mmol) at 0° C. under N2 atmosphere. The mixture was stirred at 25° C. for 16 hrs. TLC showed the reaction was completed. The solution was poured into water (40 mL), extracted with EtOAc (20 mL×3). The combined organic layers were washed with brine (20 mL×2), filtered and concentrated under reduced pressure. The residue was purified by silica-gel column chromatography (20:1-3:1) to give tert-butyl 2-(5-(2-chloro-6-cyano-4-(1-(4-hydroxyphenyl)-1-methyl-ethyl)phenoxy)pentoxy)acetate (3) (4.5 g, yield: 80.5%) as yellow oil.

2-(5-(2-chloro-6-cyano-4-(1-methyl-1-(4-((2-methylsulfanylpyrimidin-4-yl)methoxy)phenyl) ethyl)phenoxy)pentoxy)acetate (5): To a solution of tert-butyl 2-(5-(2-chloro-6-cyano-4-(1-(4-hydroxyphenyl)-1-methyl-ethyl)phenoxy)pentoxy)acetate (3) (4.0 g, 8.20 mmol) and 4-(chloromethyl)-2-methylsulfanyl-pyrimidine; hydrochloride (4) (1.90 g, 9.02 mmol) in DMF (40 mL) was added Cs₂CO₃ (6.68 g, 20.5 mmol) at 20° C. The mixture was stirred at 20° C. for 16 hrs. LCMS showed the reaction was completed. The solution was poured into water (40 mL), extracted with EtOAc (20 mL×3). The combined organic layers were washed with brine (60 mL×3), dried over Na₂SO₄, filtered and concentrated under reduced pressure. The crude was combined with another batch of EXP-19-HR0311 and purified by silica gel column to give tert-butyl 2-(5-(2-chloro-6-cyano-4-(1-methyl-1-(4-((2-methylsulfanylpyrimidin-4-yl)methoxy) phenyl) ethyl)phenoxy)pentoxy)acetate (5) (2.74 g, yield: 53.4%) as yellow oil. ¹H NMR (400 MHz, CDCl₃) δ ppm 8.53 (d, J=5.2 Hz, 1H) 7.41 (d, J=2.4 Hz, 1H) 7.29 (d, J=2.0 Hz, 1H) 7.21 (d, J=5.2 Hz, 1H) 7.10 (dd, J=6.8, 2.0 Hz, 2H) 6.89 (dd, J=6.8, 2.0 Hz, 2H) 5.09 (s, 2H) 4.18 (t, J=6.8 Hz, 2H) 3.96 (s, 2H) 3.56 (t, J=6.8 Hz, 2H) 2.59 (s, 3H) 1.90 (quin, J=7.00 Hz, 2H) 1.67-1.77 (m, 2H) 1.57-1.66 (m, 8H) 1.49 (s, 9H).

2-(5-(2-chloro-6-cyano-4-(1-methyl-1-(4-((2-methylsulfonylpyrimidin-4-yl)methoxy)phenyl) ethyl)phenoxy)pentoxy)acetate (6): To a mixture of tert-butyl 2-(5-(2-chloro-6-cyano-4-(1-methyl-1-(4-((2-methylsulfanylpyrimidin-4-yl)methoxy)phenyl)ethyl)phenoxy)pentoxy)acetate (5) (2.70 g, 4.31 mmol) in THF (30 mL) and H₂O (30 mL) was added Oxone (7.95 g, 12.9 mmoL) at 20° C. and the mixture was stirred at 30° C. for 16 hrs. LCMS showed the reaction was completed. The solution was poured into water (40 mL), extracted with EtOAc (30 mL×3). The combined organic layers were washed with brine (90 mL×3), dried over Na₂SO₄, filtered and concentrated under reduced pressure to give tert-butyl 2-(5-(2-chloro-6-cyano-4-(1-methyl-1-(4-((2-methylsulfonylpyrimidin-4-yl)methoxy)phenyl)ethyl)phenoxy)pentoxy)acetate (6) (2.55 g, yield: 89.9%) as yellow oil. ¹H NMR (400 MHz, CDCl₃) δ ppm 8.94 (d, J=5.2 Hz, 1H) 7.85 (d, J=5.2 Hz, 1H) 7.42 (d, J=2.4 Hz, 1H) 7.29 (d, J=2.4 Hz, 1H) 7.15 (d, J=8.8 Hz, 2H) 6.91 (d, J=8.8 Hz, 2H) 5.30 (s, 2H) 4.19 (t, J=6.4 Hz, 2H) 3.96 (s, 2H) 3.56 (t, J=6.8 Hz, 2H) 3.39 (s, 3H) 1.90 (quin, J=7.0 Hz, 2H) 1.68-1.77 (m, 2H) 1.59-1.67 (m, 8H) 1.49 (s, 9H).

tert-Butyl 2-(5-(2-chloro-6-cyano-4-(1-(4-((2-(methanesulfonamido)pyrimidin-4-yl) methoxy)phenyl)-1-methyl-ethyl)phenoxy)pentoxy)acetate (7): To a solution of tert-butyl 2-(5-(2-chloro-6-cyano-4-(1-methyl-1-(4-((2-methylsulfonylpyrimidin-4-yl)methoxy)phenyl)ethyl) phenoxy)pentoxy)acetate (6) (2.55 g, 3.87 mmol) and Methanesulfonamide (1.11 g, 11.6 mmol) in CH₃CN (30 mL) was added Cs₂CO₃ (3.79 g, 11.6 mmol) at 20° C. The mixture was stirred at 20° C. for 16 hrs. LCMS showed the reaction was completed. The solution was poured into water (30 mL), extracted with EtOAc (20 mL×3). The combined organic layers were washed with brine (60 mL), dried over Na₂SO₄, filtered and concentrated under reduced pressure. The residue was purified by silica gel column to give tert-butyl 2-(5-(2-chloro-6-cyano-4-(1-(4-((2-(methanesulfonamido)pyrimidin-4-yl)methoxy)phenyl)-1-methyl-ethyl)phenoxy) pentoxy) acetate (7) (1.39 g, yield: 53.3%) as yellow solid. ¹H NMR (400 MHz, CDCl₃) δ ppm 9.0 (br s, 1H) 8.65 (d, J=5.2 Hz, 1H) 7.42 (d, J=2.0 Hz, 1H) 7.28-7.32 (m, 2H) 7.13 (d, J=8.8 Hz, 2H) 6.90 (d, J=8.8 Hz, 2H) 5.11 (s, 2H) 4.18 (t, J=6.8 Hz, 2H) 3.96 (s, 2H) 3.56 (t, J=6.4 Hz, 2H) 3.48 (s, 3H) 1.84-1.95 (m, 2H) 1.67-1.77 (m, 2H) 1.59-1.67 (m, 8H) 1.49 (s, 9H).

2-(5-(2-chloro-6-cyano-4-(1-(4-((2-(methanesulfonamido)pyrimidin-4-yl)methoxy)phenyl)-1-methyl-ethyl)phenoxy)pentoxy)acetic acid (8): To a solution of tert-butyl 2-(5-(2-chloro-6-cyano-4-(1-(4-((2-(methanesulfonamido)pyrimidin-4-yl)methoxy)phenyl)-1-methyl-ethyl) phenoxy)pentoxy)acetate (7) (1.0 g, 1.49 mmol) in DCM (10 mL) was added TFA (2 mL) and the mixture was stirred at 25° C. for 6 hrs. LCMS showed the reaction was completed. The mixture was concentrated under reduced pressure. Then the crude was purified by p-HPLC (NH₄HCO₃) to give 2-(5-(2-chloro-6-cyano-4-(1-(4-((2-(methanesulfonamido)pyrimidin-4-yl) methoxy) phenyl)-1-methyl-ethyl)phenoxy)pentoxy)acetic acid (8) (0.26 g, yield: 24.1%) as yellow oil. ¹H NMR (400 MHz, CDCl₃) δ ppm 8.62 (d, J=5.2 Hz, 1H) 7.42 (d, J=2.0 Hz, 1H) 7.27-7.30 (m, 3H) 7.12 (d, J=8.8 Hz, 2H) 6.89 (d, J=8.8 Hz, 2H) 5.11 (s, 2H) 4.19 (t, J=6.0 Hz, 2H) 4.11 (s, 2H) 3.63 (t, J=6.4 Hz, 2H) 3.47 (s, 3H) 1.89 (quin, J=6.8 Hz, 2H) 1.67-1.79 (m, 4H) 1.62-1.66 (m, 7H)

(2S,4R)-1-((S)-2-(2-((5-(2-chloro-6-cyano-4-(2-(4-((2-(methylsulfonamido)pyrimidin-4-yl)methoxy)phenyl)propan-2-yl)phenoxy)pentyl)oxy)acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide: To a solution of 2-(5-(2-chloro-6-cyano-4-(1-(4-((2-(methanesulfonamido)pyrimidin-4-yl)methoxy)phenyl)-1-methyl-ethyl)phenoxy)pentoxy)acetic acid (8) (100 mg, 0.162 mmol), (2S,4R)-1-((S)-2-amino-3,3-dimethylbutanoyl)-N—((R)-2-(dimethylamino)-1-(4-(4-methylthiazol-5-yl)phenyl)ethyl)-4-hydroxypyrrolidine-2-carboxamide (79.0 mg, 0.162 mmol), HOBT (24.8 mg, 0.162 mmol) in DCM (2 mL) was added DIEA (0.139 mL, 0.810 mmol) and EDC HCl (0.0311 g, 0.162 mmol) at 20° C., then the mixture was stirred at 25° C. for 16 hrs. LCMS showed the reaction was completed. The mixture was poured into H₂O (5 mL), extracted with DCM (2 mL×3), and the combined organic layers were was washed with brine (2 mL×2), dried over Na₂SO₄, filtered and concentrated in vacuo to give the crude. The crude was purified by p-HPLC (HCl) to give (2S,4R)-1-((S)-2-(2-((5-(2-chloro-6-cyano-4-(2-(4-((2-(methylsulfonamido)pyrimidin-4-yl)methoxy)phenyl)propan-2-yl)phenoxy)pentyl)oxy) acetamido)-3,3-dimethylbutanoyl)-N—((R)-2-(dimethylamino)-1-(4-(4-methylthiazol-5-yl)phenyl)ethyl)-4-hydroxypyrrolidine-2-carboxamide (97.0% purity, 21.3 mg, yield: 11.7%) as yellow solid. ¹H NMR (400 MHz, DMSO-d₆) δ ppm 10.10 (br s, 1H) 9.29 (br d, J=8.4 Hz, 1H) 9.06 (s, 1H) 8.63 (d, J=5.0 Hz, 1H) 7.46-7.66 (m, 6H) 7.34 (d, J=5.6 Hz, 1H) 7.10-7.24 (m, 3H) 6.97 (br d, J=8.4 Hz, 2H) 6.58 (s, 1H) 5.35-5.51 (m, 1H) 5.13 (s, 2H) 4.48-4.61 (m, 5H) 4.33 (br s, 2H) 4.13 (br t, J=6.4 Hz, 2H) 3.94 (s, 2H) 3.58-3.74 (m, 2H) 3.41-3.56 (m, 4H) 3.35 (s, 3H) 2.83-3.04 (m, 6H) 2.47 (s, 3H) 2.01-2.17 (m, 1H) 1.74-1.90 (m, 3H) 1.46-1.69 (m, 9H) 0.94 (s, 9H) LCMS (220 nm): 97.0%. Exact Mass: 1085.4; found: 1086.4/1087.4.

### Example 51: N-(4-((4-(2-(3-chloro-5-cyano-4-(3-(4-(((2-(2,6-dioxopiperidin-3-yl)-1,3-dioxoisoindolin-4-yl)amino)methyl)-1H-1,2,3-triazol-1-yl)propoxy)phenyl)propan-2-yl)phenoxy)methyl)pyrimidin-2-yl)methanesulfonamide

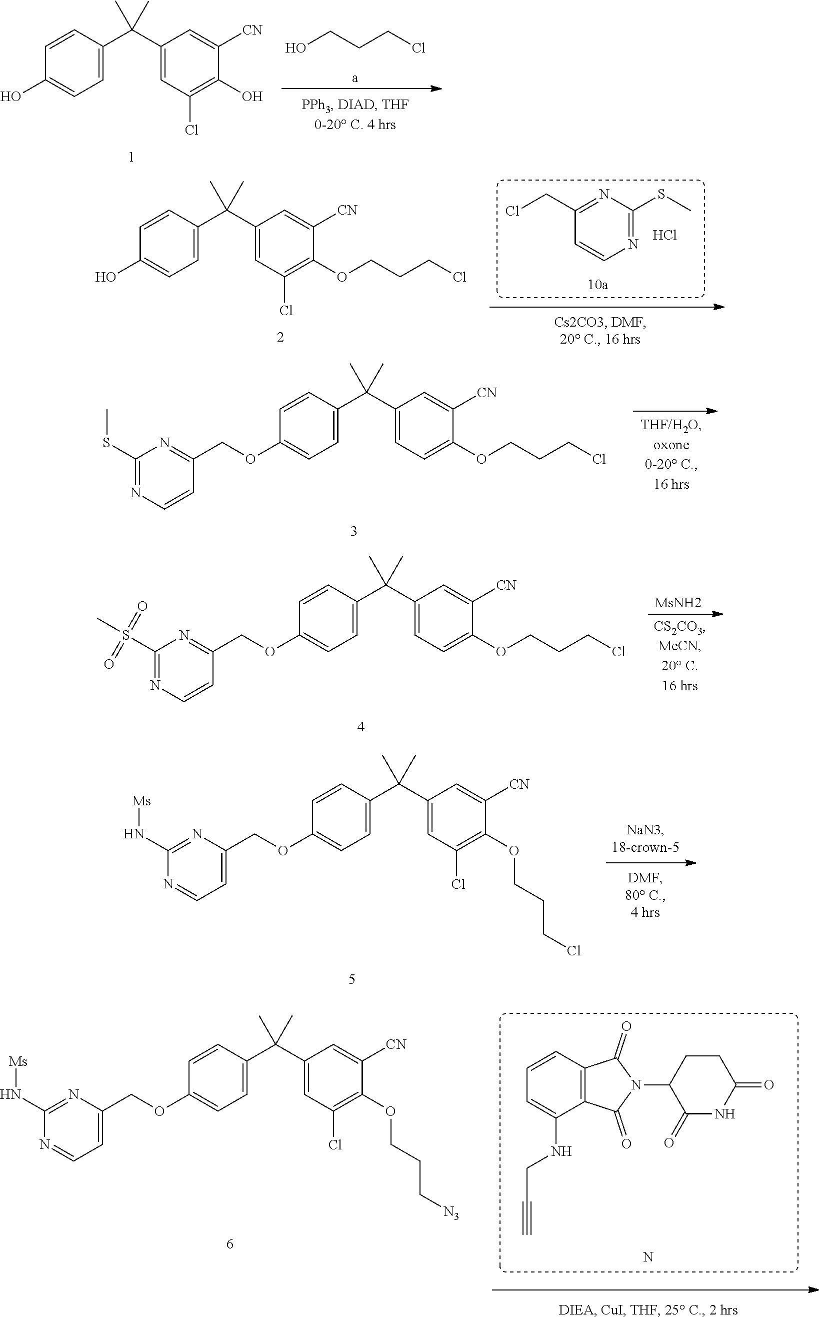

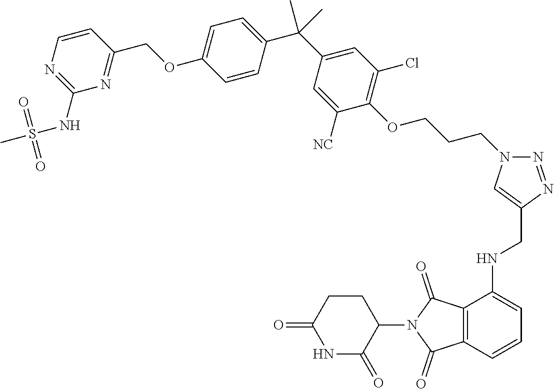 [SMILES: CC(C)(c1ccc(OCc2ccnc(NS(C)(=O)=O)n2)cc1)c1cc(Cl)c(OCCCn2cc(CNc3cccc4c3C(=O)N(C3CCC(=O)NC3=O)C4=O)nn2)c(C#N)c1]

3-Chloro-2-(3-chloropropoxy)-5-(2-(4-hydroxyphenyl)propan-2-yl)benzonitrile (2): To a solution of 3-chloro-2-hydroxy-5-[1-(4-hydroxyphenyl)-1-methyl-ethyl]benzonitrile (2.00 g, 6.26 mmol) in THF (20 mL) was added 3-chloropropan-1-ol (0.591 g, 6.26 mmol), (E)-1-tert-butyl 2-isopropyl diazene-1,2-dicarboxylate (1.85 mL, 9.38 mmol) at 0° C. The reaction was stirred at 20° C. for 4 hrs under N2. LCMS showed the reaction was completed. The mixture was quenched with water (20 mL) and extracted with EtOAc (10 mL×3). The combined organic layers were washed with brine (30 mL), dried over Na₂SO₄, filtered and concentrated under reduced pressure. The crude was purified by MPLC to give 3-chloro-2-(3-chloropropoxy)-5-(2-(4-hydroxyphenyl)propan-2-yl)benzonitrile (2.20 g, yield: 86.9%) as brown oil. ¹H NMR (400 MHz, CDCl₃) δ=7.43 (d, J=2.4 Hz, 1H), 7.33 (d, J=2.4 Hz, 1H), 7.06 (d, J=2.4 Hz, 2H), 6.79 (d, J=2.0 Hz, 2H), 4.33 (t, J=5.2 Hz, 2H), 3.86 (t, J=6.4 Hz, 2H), 2.32-2.29 (m, 2H), 1.64 (s, 6H).

3-Chloro-2-(3-chloropropoxy)-5-(2-(4-hydroxyphenyl)propan-2-yl)benzonitrile (3): To a solution of 3-chloro-2-(3-chloropropoxy)-5-[1-(4-hydroxyphenyl)-1-methyl-ethyl]benzonitrile (1.10 g, 2.72 mmol) in DMF (10 mL) was added Cs₂CO₃ (1.77 g, 5.44 mmol), 4-(chloromethyl)-2-methylsulfanyl-pyrimidine (0.828 g, 4.74 mmol) at 20° C. The reaction was stirred at 20° C. for 16 hrs under N2. LCMS showed the reaction was completed. The mixture was quenched with water (20 mL) and extracted with EtOAc (10 mL×3). The combined organic layers were washed with brine (30 mL), dried over Na₂SO₄, filtered and concentrated under reduced pressure. The crude was purified by silica gel column (PE:EtOAc=20:1-3:1) to give 3-chloro-2-(3-chloropropoxy)-5-(2-(4-((2-(methylthio)pyrimidin-4-yl) methoxy)phenyl) propan-2-yl)benzonitrile (90.0% purity, 1.10 g, yield: 72.5%) as brown oil. ¹H NMR (400 MHz, CDCl₃) δ=8.54 (s, 1H), 7.44 (d, J=2.0 Hz, 1H), 7.32 (d, J=2.4 Hz, 1H), 7.21 (d, J=5.2 Hz, 1H), 7.11 (d, J=8.8 Hz, 2H), 6.89 (d, J=8.8 Hz, 2H), 5.09 (s, 2H), 4.34 (t, J=5.6 Hz, 2H), 3.86 (t, J=6.0 Hz, 2H), 2.59 (s, 3H), 2.32-2.29 (m, 2H), 1.65 (s, 6H).

3-Chloro-2-(3-chloropropoxy)-5-(2-(4-((2-(methylsulfonyl)pyrimidin-4-yl)methoxy) phenyl)propan-2-yl)benzonitrile (4): To a solution of 3-chloro-2-(3-chloropropoxy)-5-[1-methyl-1-[4-[(2-methylsulfanylpyrimidin-4-yl)methoxy]phenyl]ethyl]benzonitrile (90.0%, 1.10 g, 1.97 mmol) in THF (10 mL)/water (10 mL) was added Oxone (3.03 g, 4.93 mmol) at 0° C. The reaction was stirred at 20° C. for 16 hrs under N2. LCMS showed the reaction was completed. The mixture was quenched with aq.Na₂SO₃ (20 mL) and extracted with EtOAc (10 mL×3). The combined organic layers were washed with brine (30 mL), dried over Na₂SO₄, filtered and concentrated under reduced pressure to give 3-chloro-2-(3-chloropropoxy)-5-(2-(4-((2-(methylsulfonyl)pyrimidin-4-yl)methoxy)phenyl)propan-2-yl)benzonitrile (90.0% purity, 1.10 g, yield: 94.0%) as brown oil. ¹H NMR (400 MHz, CDCl₃) δ=8.94 (d, J=5.2 Hz, 1H), 7.85 (d, J=5.2 Hz, 1H), 7.44 (d, J=2.4 Hz, 1H), 7.30 (d, J=2.4 Hz, 1H), 7.15 (d, J=8.8 Hz, 2H), 6.92 (d, J=8.8 Hz, 2H), 5.29 (s, 2H), 4.34 (t, J=5.6 Hz, 2H), 3.86 (t, J=6.4 Hz, 2H), 3.39 (s, 3H), 2.32-2.29 (m, 2H), 1.65 (s, 6H).

N-(4-((4-(2-(3-Chloro-4-(3-chloropropoxy)-5-cyanophenyl)propan-2-yl)phenoxy) methyl) pyrimidin-2-yl)methanesulfonamide (5): To a solution of 3-chloro-2-(3-chloropropoxy)-5-[1-methyl-1-[4-[(2-methylsulfonylpyrimidin-4-yl)methoxy]phenyl]ethyl]benzonitrile (90.0% purity, 1.10 g, 0.185 mmol) in MeCN (10 mL) was added Cs₂CO₃ (1.81 g, 0.556 mmol) and methanesulfonamide (0.529 g, 0.556 mmol) into the reaction at 20° C. The reaction was stirred at 20° C. for 16 hrs under N₂. LCMS showed the reaction was completed. The mixture was quenched with water (20 mL) and extracted with EtOAc (10 mL×3). The combined organic layers were washed with brine (30 mL), dried over Na₂SO₄, filtered and concentrated under reduced pressure. The crude was purified by silica gel column (PE:EtOAc=20:1-1:1) to give N-(4-((4-(2-(3-chloro-4-(3-chloropropoxy)-5-cyanophenyl)propan-2-yl)phenoxy)methyl)pyrimidin-2-yl)methanesulfonamide (90.0% purity, 2.10 g, yield: 74.6%) as brown oil. ¹H NMR (400 MHz, CDCl₃) δ=8.38 (s, 1H), 7.40 (d, J=1.6 Hz, 1H), 7.05 (d, J=8.0 Hz, 1H), 6.90 (d, J=9.2 Hz, 2H), 6.82 (d, J=7.6 Hz, 3H), 4.91 (s, 2H), 4.32 (t, J=5.6 Hz, 2H), 3.84 (t, J=6.0 Hz, 2H), 3.07 (s, 3H), 2.31-2.25 (m, 2H), 1.59 (s, 6H).

N-(4-((4-(2-(4-(3-Azidopropoxy)-3-chloro-5-cyanophenyl)propan-2-yl)phenoxy)methyl) pyrimidin-2-yl)methanesulfonamide (6): To a solution of N-[4-[[4-[1-[3-chloro-4-(3-chloropropoxy)-5-cyano-phenyl]-1-methyl-ethyl]phenoxy]methyl]pyrimidin-2-yl]methanesulfonamide (99.3% purity, 0.200 g, 0.361 mmol) in DMF (2 mL) was added 18-Crown-6 (0.0287 g, 0.108 mmol), NaN₃ (0.0705 g, 1.08 mmol) at 20° C. The reaction was stirred at 80° C. for 4 hrs under N2. LCMS showed the reaction was completed. The mixture was quenched with water (10 mL) and extracted with EtOAC (5 mL×3). The combined organic layers were washed with water (20 mL×4), dried over Na₂SO₄, filtered and concentrated under reduced pressure to give N-(4-((4-(2-(4-(3-azidopropoxy)-3-chloro-5-cyanophenyl)propan-2-yl)phenoxy)methyl)pyrimidin-2-yl)methanesulfonamide (90.0% purity, 0.170 g, 0.275 mmol, yield: 76.1%) as brown oil. ¹H NMR (400 MHz, CDCl₃) δ=9.34 (s, 1H), 8.66 (d, J=5.2 Hz, 1H), 7.44 (d, J=2.4 Hz, 1H), 7.31 (d, J=2.0 Hz, 1H), 7.12 (d, J=8.8 Hz, 2H), 6.90 (d, J=8.8 Hz, 2H), 5.12 (s, 2H), 4.26 (t, J=5.6 Hz, 2H), 3.66 (t, J=6.8 Hz, 2H), 3.47 (s, 3H), 2.14-2.07 (m, 2H), 1.64 (s, 6H).

N-(4-((4-(2-(3-chloro-5-cyano-4-(3-(4-(((2-(2,6-dioxopiperidin-3-yl)-1,3-dioxoisoindolin-4-yl)amino)methyl)-1H-1,2,3-triazol-1-yl)propoxy)phenyl)propan-2-yl)phenoxy)methyl) pyrimidin-2-yl)methanesulfonamide: To a solution of 2-(2,6-dioxo-3-piperidyl)-4-(prop-2-ynylamino)isoindoline-1,3-dione (0.143 g, 0.459 mmol) in THF (2 mL) was added N-[4-[[4-[1-[4-(3-azidopropoxy)-3-chloro-5-cyano-phenyl]-1-methyl-ethyl]phenoxy]methyl]pyrimidin-2-yl]methanesulfonamide (0.170 g, 0.306 mmol), CuI (0.0291 g, 0.153 mmol), DIEA (0.105 mL, 0.611 mmol) at 20° C. The reaction was stirred at 20° C. for 4 hrs under N2. LCMS showed the reaction was completed. The mixture was quenched with water (10 mL) and extracted with EtOAc (5 mL×3). The combined organic layers were washed with brine (10 mL), dried over Na₂SO₄, filtered and concentrated under reduced pressure. The crude was purified by p-HPLC (NH₄HCO₃) to give N-(4-((4-(2-(3-chloro-5-cyano-4-(3-(4-(((2-(2,6-dioxopiperidin-3-yl)-1,3-dioxoisoindolin-4-yl)amino)methyl)-1H-1,2,3-triazol-1-yl)propoxy)phenyl)propan-2-yl)phenoxy)methyl)pyrimidin-2-yl)methanesulfonamide (96.0% purity, 0.0172 g, yield: 6.23%) as brown oil. ¹H NMR (400 MHz, CDCl₃) δ=9.31 (s, 1H), 8.61 (d, J=5.2 Hz, 1H), 7.74 (s, 1H), 7.41 (d, J=2.4 Hz, 1H), 7.30 (s, 1H), 7.28 (d, J=10.0 Hz, 1H), 7.10 (d, J=8.8 Hz, 3H), 7.07-7.04 (m, 1H), 6.88 (d, J=8.8 Hz, 2H), 6.72 (s, 1H), 5.12 (s, 2H), 4.94-4.70 (m, 1H), 4.65 (t, J=6.0 Hz, 2H), 4.14-4.07 (m, 2H), 3.46 (s, 3H), 2.93 (d, J=15.2 Hz, 1H), 2.82-2.76 (m, 2H), 2.48 (d, J=6.0 Hz, 2H), 2.15 (d, J=7.6 Hz, 2H), 1.65 (s, 6H). LCMS: (220 nm): 96.2%. Exact Mass: 866.2; found 867.2/869.2.

### Example 52: Synthesis of (2S,4R)-1-[(2S)-2-[2-({5-[2-({4-[(4-{2-[3-chloro-4-(2-chloroethoxy)-5-cyanophenyl]propan-2-yl}phenoxy)methyl]pyrimidin-2-yl}sulfamoyl)ethoxy]pentyl}oxy)acetamido]-3,3-dimethylbutanoyl]-N-[(1R)-2-(dimethylamino)-1-[4-(4-methyl-1,3-thiazol-5-yl)phenyl]ethyl]-4-hydroxypyrrolidine-2-carboxamide

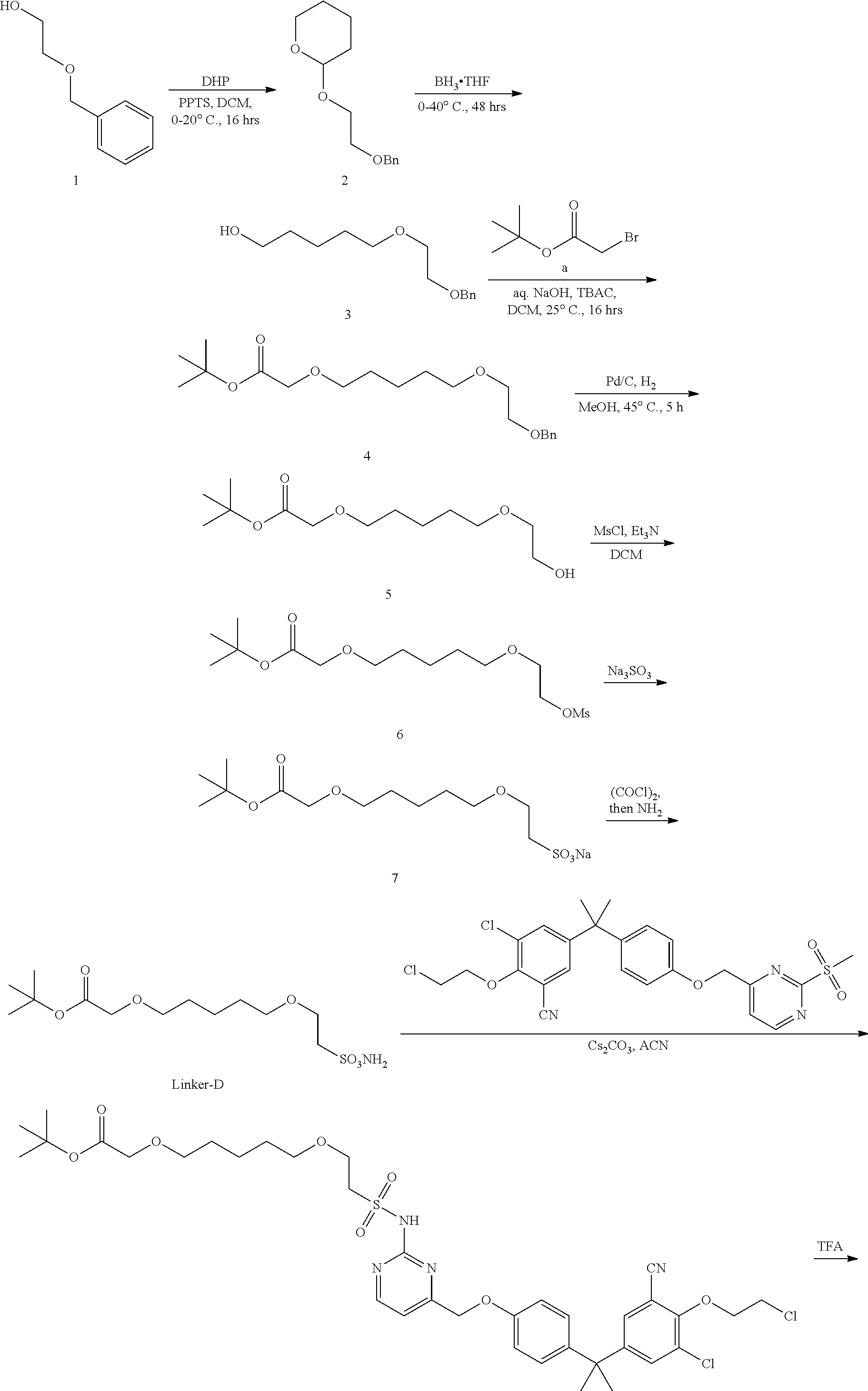

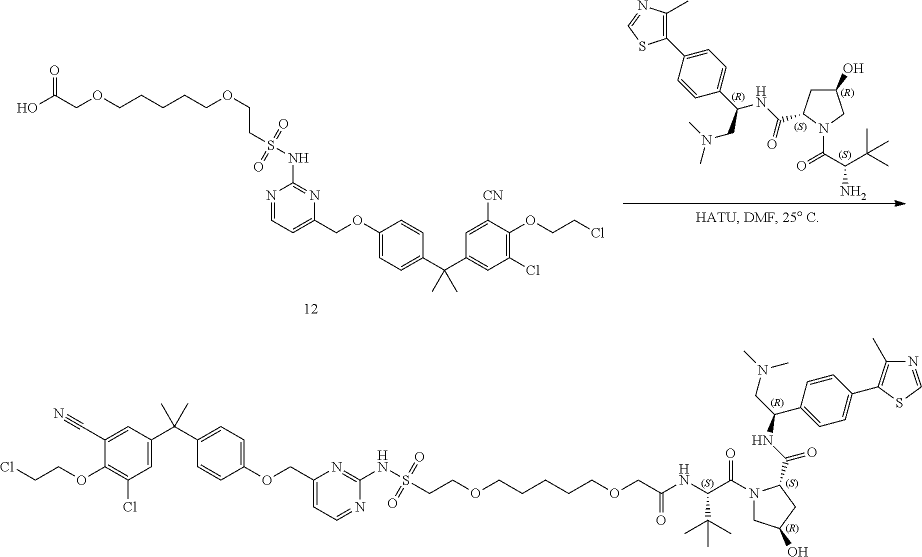

The titled compound is synthesized according to the scheme shown above.

### Example 53: Synthesis of (2S,4R)-1-[(2S)-2-[2-({5-[2-({4-[(4-{2-[3-chloro-4-(2-chloroethoxy)-5-cyanophenyl]propan-2-yl}phenoxy)methyl]pyrimidin-2-yl}sulfamoyl)ethoxy]pentyl}oxy)acetamido]-3,3-dimethylbutanoyl]-4-hydroxy-N-{[4-(4-methyl-1,3-thiazol-5-yl)phenyl]methyl}pyrrolidine-2-carboxamide

Titled compound is synthesized according to Example 52 with the modification of the last step as follows:

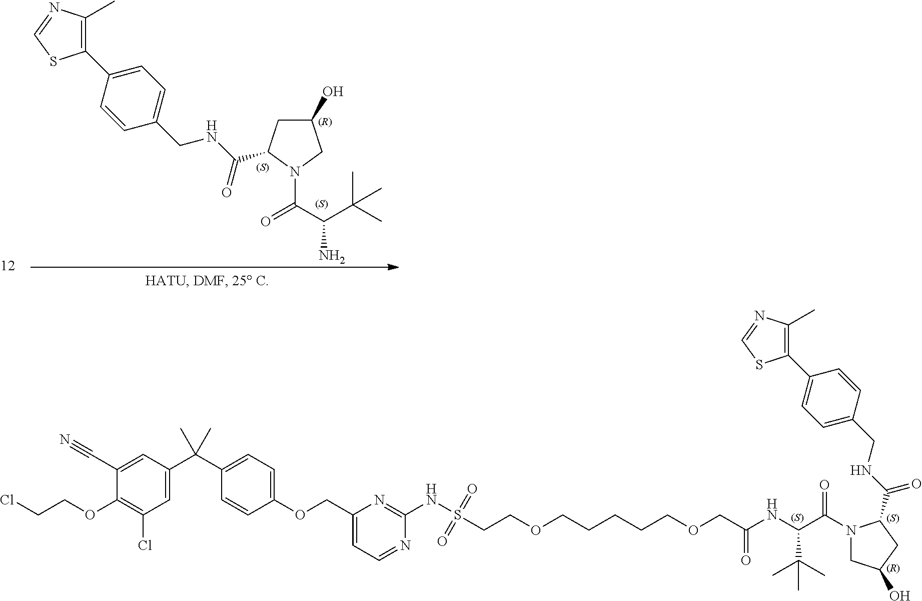

### Example 54: Synthesis of (2S,4S)-1-[(2S)-2-[2-({5-[2-({4-[(4-{2-[3-chloro-4-(2-chloroethoxy)-5-cyanophenyl]propan-2-yl}phenoxy)methyl]pyrimidin-2-yl}sulfamoyl)ethoxy]pentyl}oxy)acetamido]-3,3-dimethylbutanoyl]-N-[(1R)-2-(dimethylamino)-1-[4-(4-methyl-1,3-thiazol-5-yl)phenyl]ethyl]-4-hydroxypyrrolidine-2-carboxamide

Titled compound is synthesized according to Example 52 with the modification of the last step as follows:

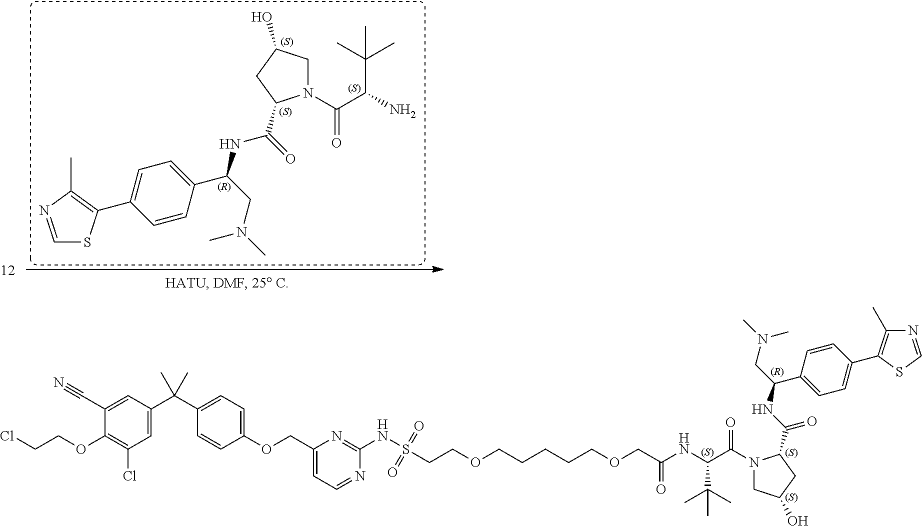

### Example 55: Synthesis of (2S,4S)-1-[(2S)-2-[2-({5-[2-({4-[(4-{2-[3-chloro-4-(2-chloroethoxy)-5-cyanophenyl]propan-2-yl}phenoxy)methyl]pyrimidin-2-yl}sulfamoyl)ethoxy]pentyl}oxy)acetamido]-3,3-dimethylbutanoyl]-4-hydroxy-N-{[4-(4-methyl-1,3-thiazol-5-yl)phenyl]methyl}pyrrolidine-2-carboxamide

Titled compound is synthesized according to Example 52 with the modification of the last step as follows:

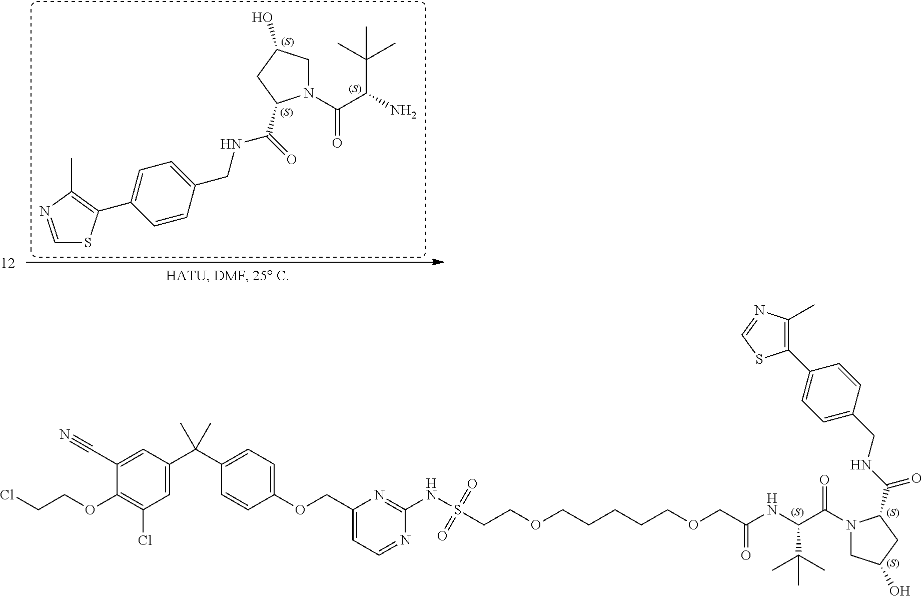

### Example 56: Synthesis of (2S,4R)-1-[(2S)-2-[2-({5-[2-({5-[(4-{2-[3-chloro-4-(2-chloroethoxy)-5-cyanophenyl]propan-2-yl}phenoxy)methyl]pyrimidin-2-yl}sulfamoyl)ethoxy]pentyl}oxy)acetamido]-3,3-dimethylbutanoyl]-N-[(1R)-2-(dimethylamino)-1-[4-(4-methyl-1,3-thiazol-5-yl)phenyl]ethyl]-4-hydroxypyrrolidine-2-carboxamide

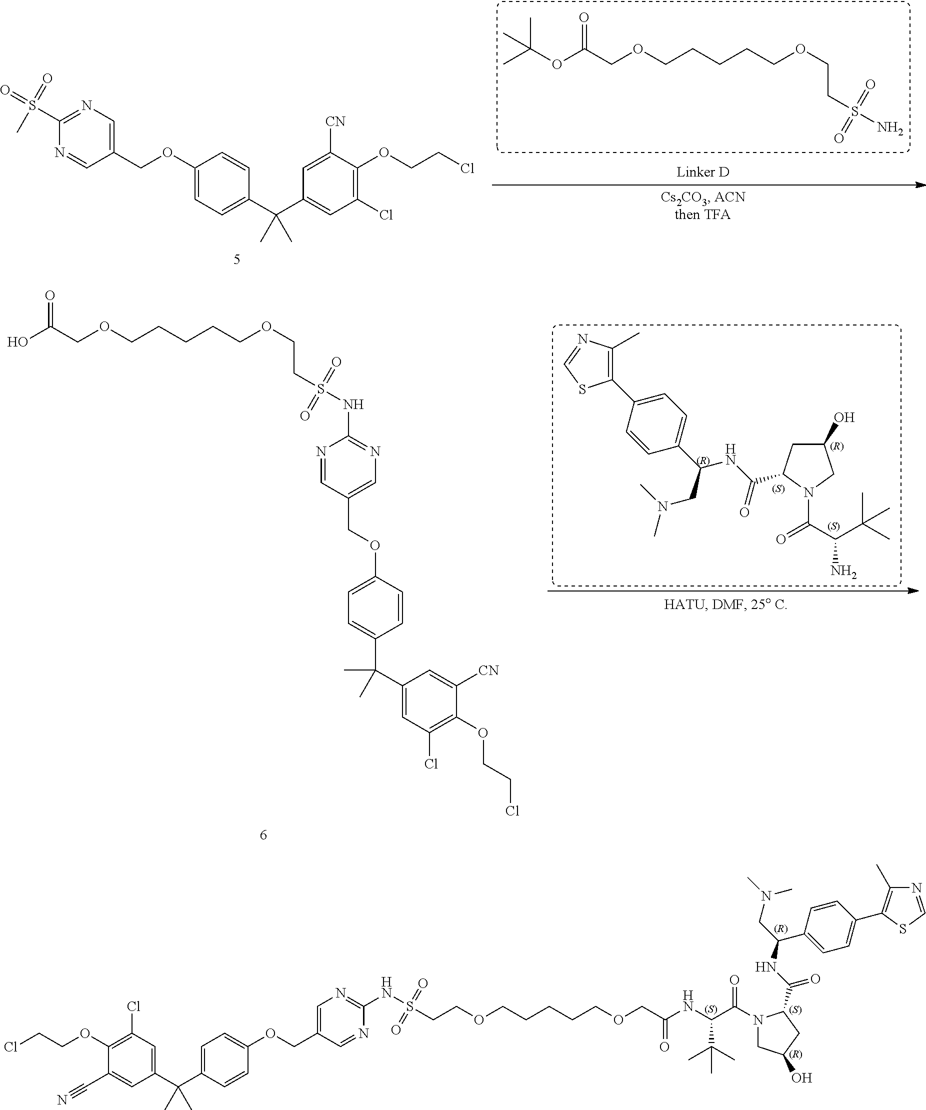

The titled compound is synthesized according to the scheme shown above.

### Example 57: Synthesis of (2S,4R)-1-[(2S)-2-{2-[(5-{[2-({4-[(4-{2-[3-chloro-4-(2-chloroethoxy)-5-cyanophenyl]propan-2-yl}phenoxy)methyl]pyrimidin-2-yl}sulfamoyl)ethyl]amino}pentyl)oxy]acetamido}-3,3-dimethylbutanoyl]-4-hydroxy-N-{[4-(4-methyl-1,3-thiazol-5-yl)phenyl]methyl}pyrrolidine-2-carboxamide

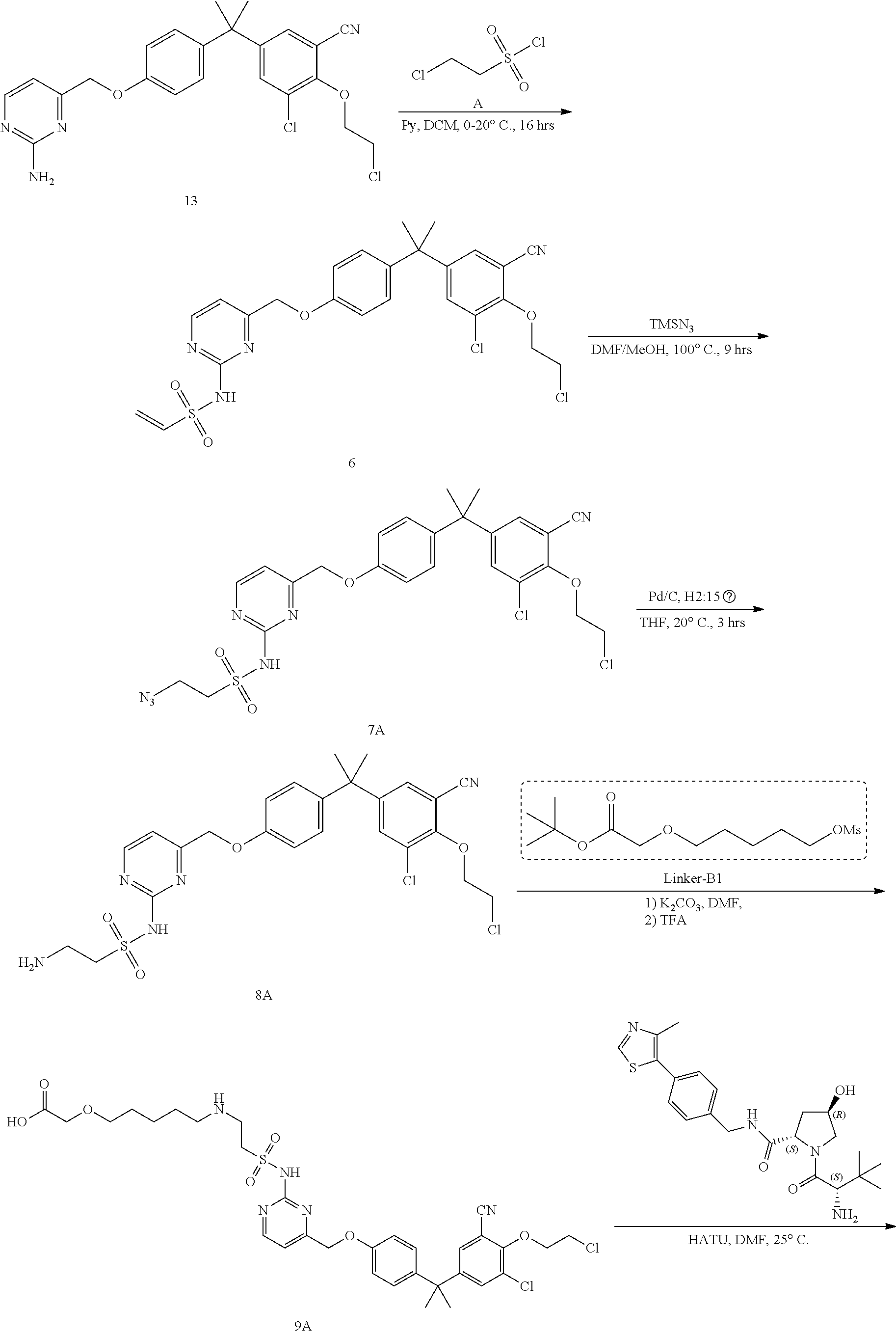

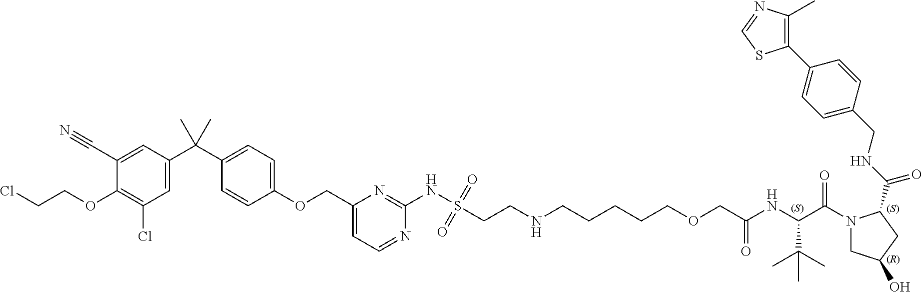

The titled compound is synthesized according to the scheme shown above.

### Example 58: Synthesis of (2S,4R)-1-[(2S)-2-(2-{[5-(5-{2-[3-chloro-4-(2-chloroethoxy)-5-cyanophenyl]propan-2-yl}-2-[(2-methanesulfonamidopyrimidin-4-yl)methoxy]phenoxy)pentyl]oxy}acetamido)-3,3-dimethylbutanoyl]-4-hydroxy-N-{[4-(4-methyl-1,3-thiazol-5-yl)phenyl]methyl}pyrrolidine-2-carboxamide

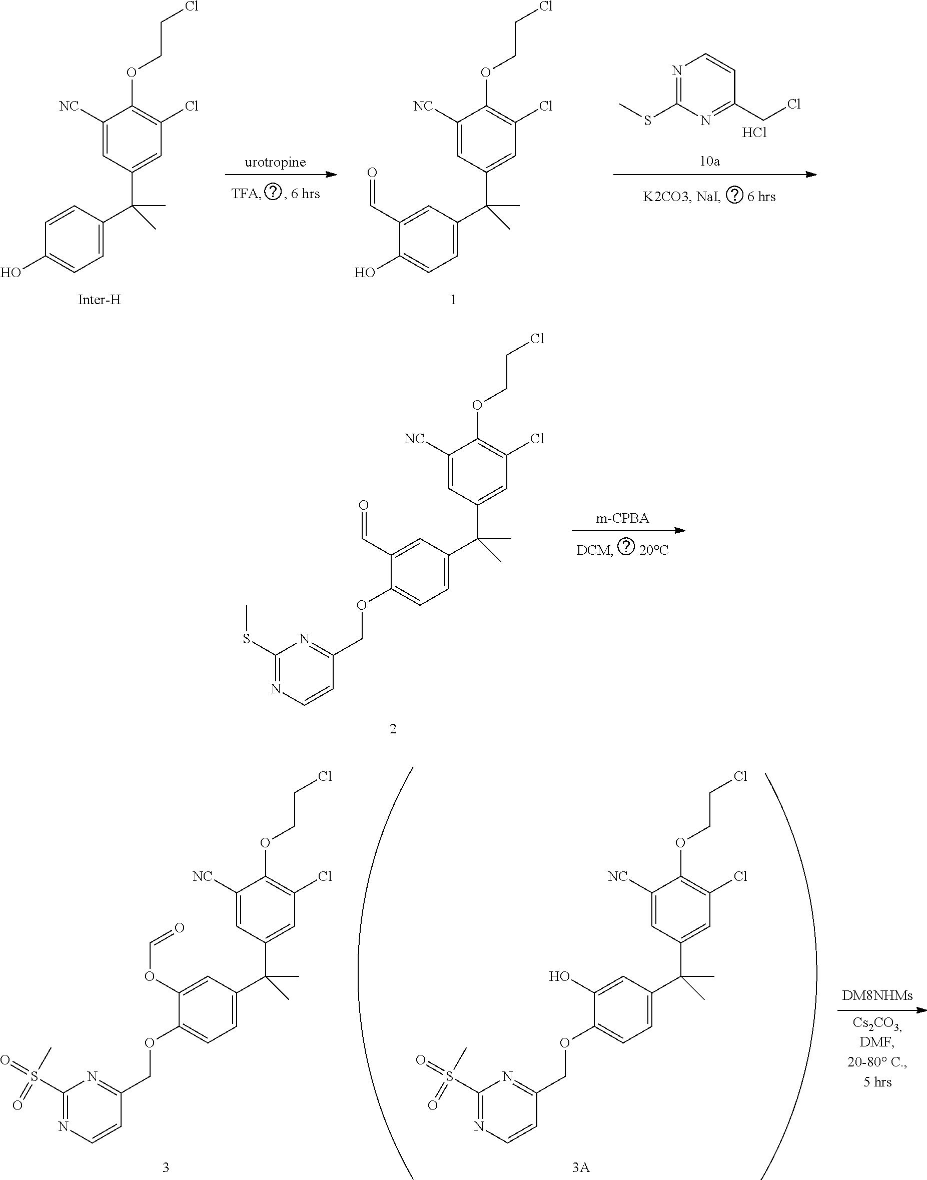

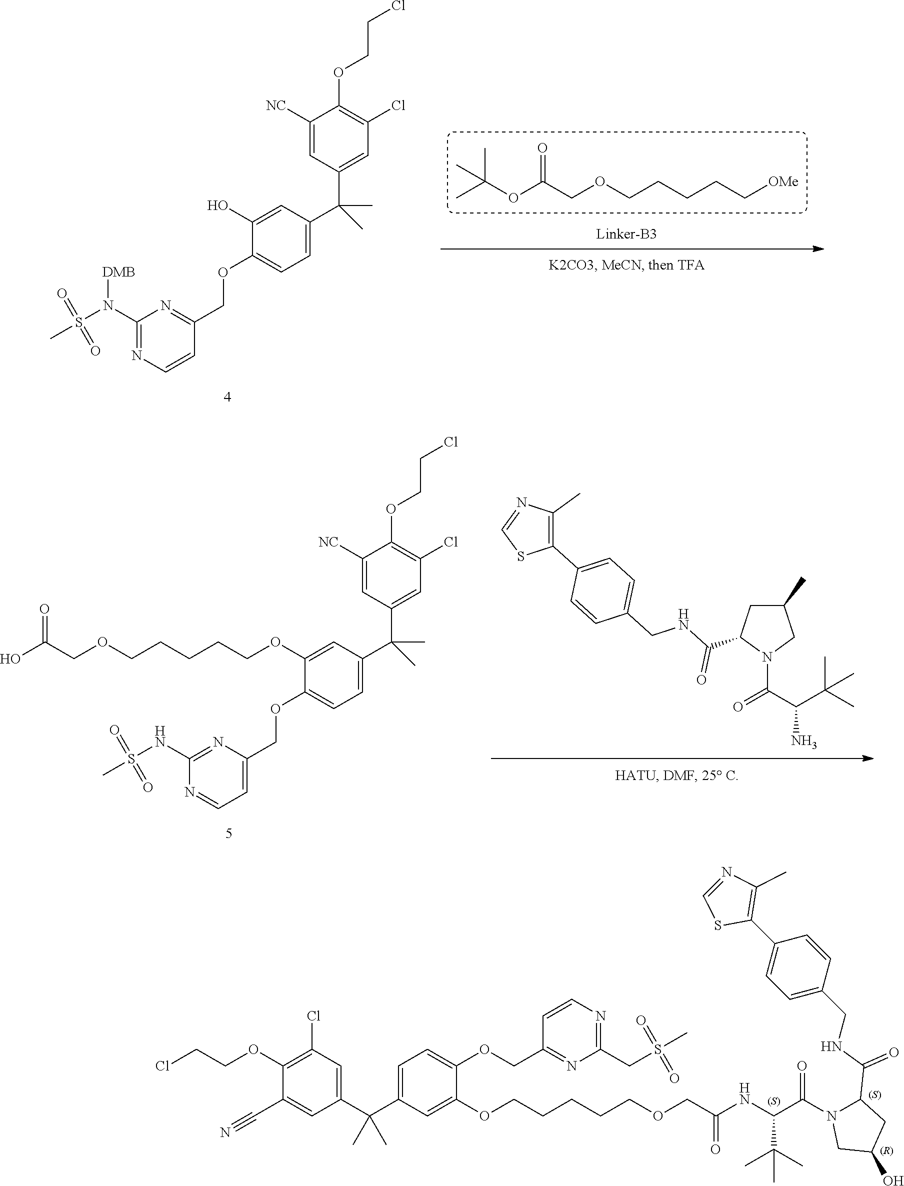

The titled compound is synthesized according to the scheme shown above.

### Example 59: Synthesis of (2S,4R)-1-[(2S)-2-(2-{3-[(5-{[2-chloro-6-cyano-4-(2-{4-[(2-methanesulfonamidopyrimidin-4-yl)methoxy]phenyl}propan-2-yl)phenyl]amino}pentyl)oxy]propoxy}acetamido)-3,3-dimethylbutanoyl]-4-hydroxy-N-{[4-(4-methyl-1,3-thiazol-5-yl)phenyl]methyl}pyrrolidine-2-carboxamide

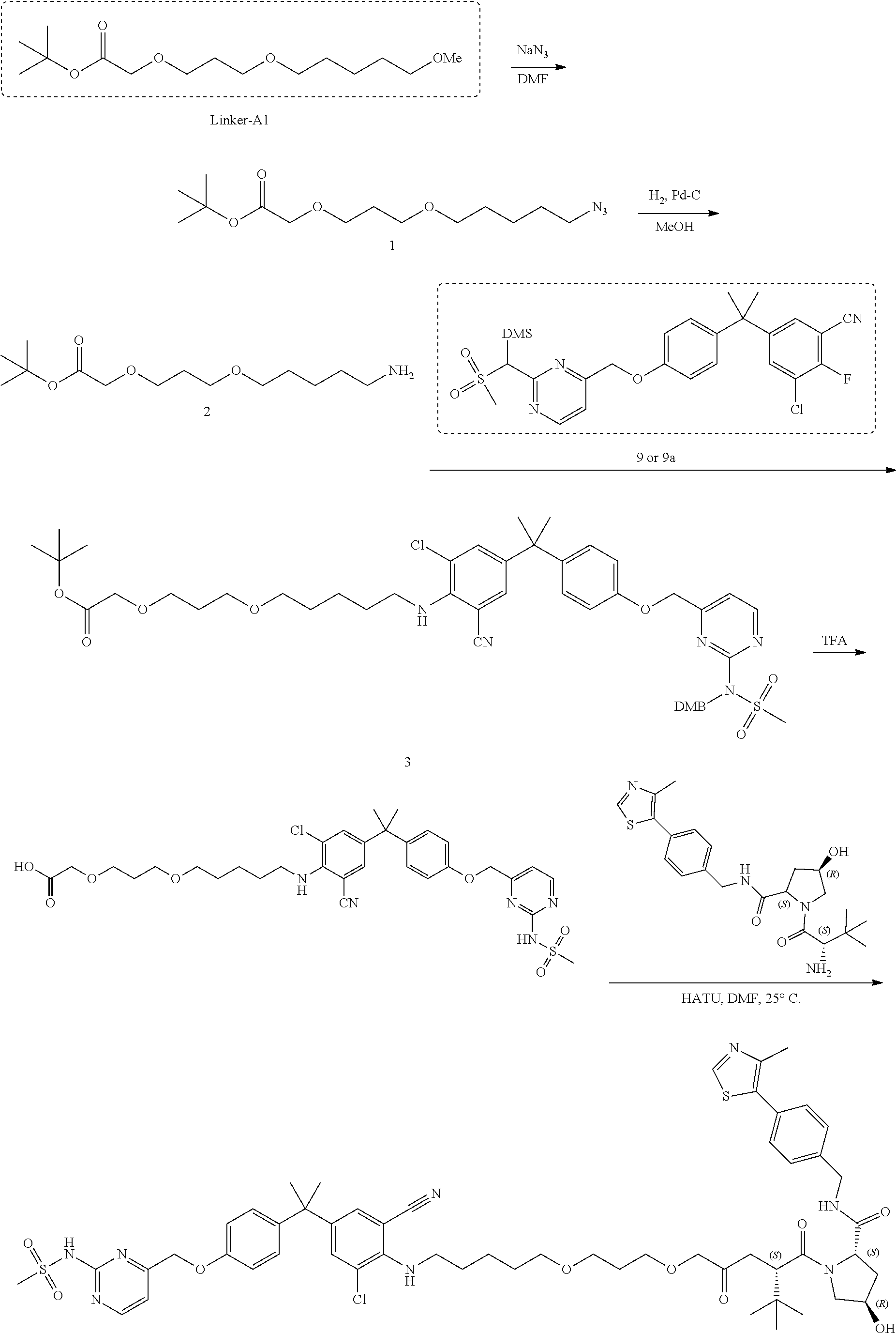

The titled compound is synthesized according to the scheme shown above.

### Example 60: 2-azido-N-(4-((4-(2-(3-chloro-4-(2-chloroethoxy)-5-cyanophenyl)propan-2-yl)phenoxy)methyl)pyrimidin-2-yl) ethanesulfonamide

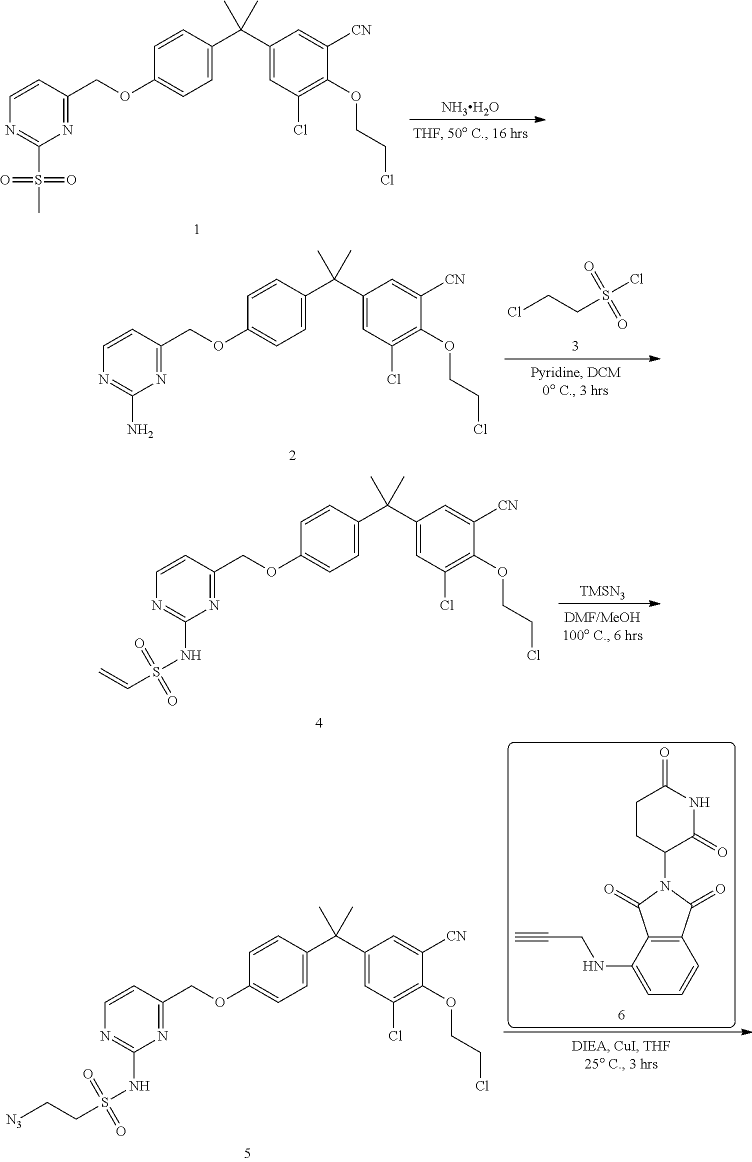

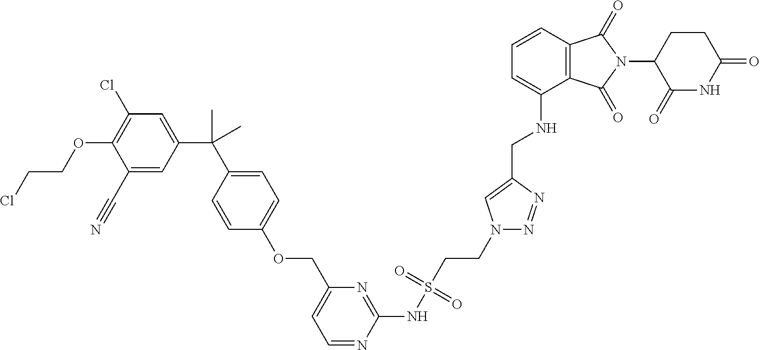 [SMILES: CC(C)(c1ccc(OCc2ccnc(NS(=O)(=O)CCn3cc(CNc4cccc5c4C(=O)N(C4CCC(=O)NC4=O)C5=O)nn3)n2)cc1)c1cc(Cl)c(OCCCl)c(C#N)c1]

5-(2-(4-((2-aminopyrimidin-4-yl)methoxy)phenyl)propan-2-yl)-3-chloro-2-(2-chloroethoxy) benzonitrile (2): To a solution of 3-chloro-2-(2-chloroethoxy)-5-(2-(4-((2-(methylsulfonyl) pyrimidin-4-yl)methoxy)phenyl)propan-2-yl)benzonitrile (1) (10 g, 19.2 mmol) in THF (100 mL) was added NH₃.H₂O (100 mL) at 20° C. The mixture was stirred at 50° C. under N2 for 16 hrs. LCMS showed the reaction was completed. The mixture was poured into water (400 mL) (lot of solid appeared), then filtered and the filter cake was concentrated under reduced pressure to give 5-(2-(4-((2-aminopyrimidin-4-yl)methoxy)phenyl)propan-2-yl)-3-chloro-2-(2-chloroethoxy) benzonitrile (2) (8.7 g, yield: 97.3%) as yellow solid. ¹H NMR (400 MHz, CDCl₃) δ=8.32 (d, J=5.2 Hz, 1H), 7.44 (d, J=2.4 Hz, 1H), 7.33 (d, J=2.4 Hz, 1H), 7.11 (d, J=8.8 Hz, 2H), 6.93-6.82 (m, 3H), 5.08 (br s, 2H), 4.96 (s, 2H), 4.42 (t, J=6.4 Hz, 2H), 3.88 (t, J=6.4 Hz, 2H), 1.64 (s, 6H).

N-(4-((4-(2-(3-chloro-4-(2-chloroethoxy)-5-cyanophenyl)propan-2-yl)phenoxy)methyl)pyrimidin-2-yl)ethenesulfonamide (4): To a solution of 5-(2-(4-((2-aminopyrimidin-4-yl)methoxy)phenyl)propan-2-yl)-3-chloro-2-(2-chloroethoxy)benzonitrile (2) (5.0 g, 10.9 mmol) and Pyridine (1.73 g, 21.9 mmol) in DCM (100 mL) was added 2-chloroethanesulfonylchloride (3) (2.67 g, 16.4 mmol) at 0° C. and stirred at the same temperature for 3 hrs. TLC showed the reaction was completed. The mixture was poured into water (200 mL) and extracted with DCM (100 mL×3) and the combined organic layers were washed with brine (100 mL×2), dried over Na₂SO₄, filtered and concentrated under reduced pressure. The residue was purified by silica gel column to give N-(4-((4-(2-(3-chloro-4-(2-chloroethoxy)-5-cyanophenyl)propan-2-yl)phenoxy)methyl) pyrimidin-2-yl)ethenesulfonamide (4) (1.0 g, yield: 15.7%) as white solid. ¹H NMR (400 MHz, CDCl₃) δ=10.49 (br s, 1H), 8.69 (d, J=5.2 Hz, 1H), 7.45 (d, J=2.4 Hz, 1H), 7.32 (d, J=2.4 Hz, 1H), 7.29 (d, J=5.2 Hz, 1H), 7.15-7.11 (d, J=8.8 Hz, 2H), 7.11-7.03 (m, 1H), 6.90 (d, J=8.8 Hz, 2H), 6.55 (d, J=16.4 Hz, 1H), 6.10 (d, J=10.0 Hz, 1H), 5.11 (s, 2H), 4.42 (t, J=6.0 Hz, 2H), 3.88 (t, J=6.0 Hz, 2H), 1.65 (s, 6H).

2-azido-N-(4-((4-(2-(3-chloro-4-(2-chloroethoxy)-5-cyanophenyl)propan-2-yl)phenoxy) methyl)pyrimidin-2-yl)ethanesulfonamide (5): To a solution of N-(4-((4-(2-(3-chloro-4-(2-chloroethoxy)-5-cyanophenyl)propan-2-yl)phenoxy)methyl)pyrimidin-2-yl)ethenesulfonamide (4) (900 mg, 1.64 mmol) in DMF (2.5 mL) and MeOH (2.5 mL) was added azido(trimethyl)silane (2.5 mL) and stirred at 100° C. for 6 hrs under N2. LCMS showed the reaction was completed. The mixture was poured into water (10 mL) and extracted with EtOAc (5 mL×3), the combined organic layers were washed with brine (10 mL×4), dried over Na₂SO₄, filtered and concentrated under reduced pressure. The residue was purified by silica gel column to give 2-azido-N-(4-((4-(2-(3-chloro-4-(2-chloroethoxy)-5-cyanophenyl)propan-2-yl)phenoxy)methyl)pyrimidin-2-yl) ethanesulfonamide (5) (350 mg, yield: 34.3%) as yellow oil. ¹H NMR (400 MHz, CDCl₃) δ=9.03 (br s, 1H), 8.57 (d, J=5.2 Hz, 1H), 7.37 (d, J=2.0 Hz, 1H), 7.27-7.22 (m, 2H), 7.05 (d, J=8.8 Hz, 2H), 6.82 (d, J=8.4 Hz, 2H), 5.04 (s, 2H), 4.35 (t, J=6.0 Hz, 2H), 3.86-3.73 (m, 6H), 1.57 (s, 6H).

2-azido-N-(4-((4-(2-(3-chloro-4-(2-chloroethoxy)-5-cyanophenyl) propan-2-yl) phenoxy) methyl) pyrimidin-2-yl) ethanesulfonamide: To a solution of 2-azido-N-(4-((4-(2-(3-chloro-4-(2-chloroethoxy)-5-cyanophenyl)propan-2-yl)phenoxy)methyl)pyrimidin-2-yl) ethanesulfonamide (5) (150 mg, 0.25 mmol), 2-(2,6-dioxo-3-piperidyl)-4-(prop-2-ynylamino)isoindoline-1,3-dione (6) (79.1 mg, 0.25 mmol) and DIEA (0.0870 mL, 0.51 mmol) in THF (3 mL) was added CuI (24.3 mg, 0.13 mmol) and stirred at 25° C. for 3 hrs under N2. LCMS showed the reaction was completed. The mixture was poured into water (5 mL) and extracted with EtOAc (5 mL×3), the combined organic layers were washed with brine (5 mL×2), dried over Na₂SO₄, filtered and concentrated under reduced pressure. The residue was purified by p-HPLC (FA) to give 2-azido-N-(4-((4-(2-(3-chloro-4-(2-chloroethoxy)-5-cyanophenyl) propan-2-yl) phenoxy) methyl) pyrimidin-2-yl) ethanesulfonamide (purity: 95%, 67.5 mg, yield: 28.0%) as yellow solid. 32.5 mg was delivered. ¹H NMR (400 MHz, CDCl₃) δ=9.26-8.96 (m, 1H), 8.53 (br d, J=5.2 Hz, 1H), 7.65 (br s, 1H), 7.45 (d, J=2.0 Hz, 1H), 7.44-7.37 (m, 1H), 7.32 (d, J=2.2 Hz, 1H), 7.23 (br d, J=4.8 Hz, 1H), 7.12 (br d, J=8.2 Hz, 2H), 7.06 (br t, J=6.6 Hz, 1H), 6.96-6.86 (m, 3H), 6.62 (br s, 1H), 5.06 (s, 2H), 4.97-4.90 (m, 1H), 4.85 (br s, 2H), 4.50 (br s, 2H), 4.42 (t, J=6.4 Hz, 2H), 4.26 (s, 2H), 3.87 (t, J=6.0 Hz, 2H), 2.89-2.66 (m, 3H), 2.10 (br s, 1H), 1.64 (s, 6H). LCMS (220 nm): 95.35%, Exact Mass: 900.20, Founded: 901.2/903.2.

### BIOLOGICAL ASSAYS

### Example 61: Activity of Exemplary Compounds in Cellular Assays

LNCaP cells were transiently transfected with the PSA (6.1 kb)-luciferase reporter for 24 h, and then treated with indicated concentration of representative compounds with synthetic androgen, R1881 (1 nM) for 24 h. After 24 h of incubation with R1881, the cells were harvested, and relative luciferase activities were determined. To determine the IC₅₀, treatments were normalized to the maximum activity with androgen-induction (in the absence of test compounds, vehicle only) (Table 1).

Luciferase Assay: Lysates were thawed on ice then collected into V-bottom 96-well tissue culture plates. Lysates were centrifuged at 4° C. for 5 minutes at 4000 rpm. To measure luminescence of LNCaP cell lysates the Firefly Luciferase Assay System (Promega) was employed, according to manufacturer's protocol.

Statistical analyses were performed using GraphPad Prism (Version 6.01 for Windows; La Jolla, Calif., USA). Comparisons between treatment and control groups were compared using Two-Way ANOVA with post-hoc Dunnett's and Tukey's tests. Differences were considered statistically significant at P values less than 0.05. Densitometric quantification of relative AR levels was determined by Image.

Table 1 shows the IC₅₀ of representative Compounds from Tables A-D from androgen-induced PSA-luciferase assay. EPI-002 have the following structures:

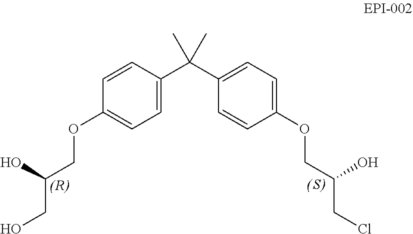

TABLE 1
IC₅₀ of Representative Compounds on Androgen-Induced
PSA in Luciferase Activity

| | Androgen-induced | |
|---|---|---|
| Compound ID | PSA-luciferase IC₅₀ (nM) | n |
| A13 | 592 | 8 |
| A28 | 400 | 5 |
| A29 | 466 | 5 |
| A35 | 515 | 6 |
| A38 | 631 | 6 |
| A66 | 890 | 6 |
| A74 | 658 | 6 |
| A93 | 205 | 4 |
| A109 | 535 | 2 |
| A122 | 258 | 2 |
| A126 | 629 | 1 |
| A131 | 1100 | 1 |
| A136 | 601 | 2 |
| A170 | 651 | 2 |
| AA31 | 51 | 5 |
| AA33 | 38 | 6 |
| AA52 | 74 | 3 |
| AA56 | 344 | 3 |
| AA85 | 368 | 1 |
| 1a | 1410 | 11 |
| 5a | 1030 | 6 |
| 9a | 3120 | 11 |
| 11a | 1050 | 10 |
| 12a | 2260 | 4 |
| 13a | 1054 | 3 |
| 14a | 950 | 11 |
| EPI-002 | 9580 | 2 |
| Enzalutamide | 189 | 8 |
| Bicalutamide | 306 | 2 |

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### NUMBERED EMBODIMENTS

### Embodiment 1

A compound of formula (Q):

PLM-LI-PTC (Q);

or a pharmaceutically acceptable salt thereof, wherein:
PLM is a E3 ligase binding group,
LI is a linker, and
PTC is an androgen receptor modulator represented by formula (IIIA):

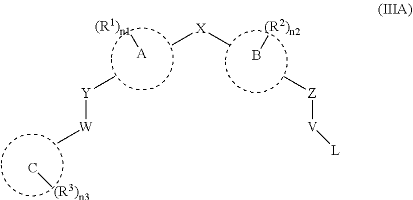

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

### Embodiment 2

The compound of embodiment 1, wherein the linker LI corresponds to formula

-LX_{A}-(CH₂)ₘ₁—(CH₂—CH₂-LX_{B})ₘ₂—(CH₂)ₘ₃-LX_{C}-, wherein:
-LX_{A} is covalently bound to the PTC or PLM, and LX_{C}- is covalently bound to the PLM or PTC;
each m1 and m2 is independently 0, 1, 2, 3, 4, 5, 6, 7, 8, 9, 10, 11, or 12;
m3 is independently 1, 2, 3, 4, 5, 6, 7, 8, 9, 10, 11, or 12;
LX_{A} is absent (a bond), —CH₂C(O)NR²⁰—, or —NR²⁰C(O)CH₂—;
LX_{B} and LX_{C} are each independently absent (a bond), —CH₂—, —O—, —S—, —S(O)—, —S(O)₂, or —N(R²⁰)—;
wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

### Embodiment 3

The compound of embodiment 2, wherein LX_{A} is absent (a bond), —CH₂C(O)NR²⁰—, or —NR²⁰C(O)CH₂—; wherein R²⁰ is hydrogen or C₁-C₃ alkyl.

### Embodiment 4

The compound of embodiment 2 or 3, wherein LX_{B} is absent (a bond), —CH₂—, —O— or —N(R²⁰)—; wherein R²⁰ is hydrogen or C₁-C₃ alkyl.

### Embodiment 5

The compound of any one of embodiments 2-4, wherein LX_{C} is absent (a bond), —CH₂—, —O—, or —NH—.

### Embodiment 6

The compound of any one of embodiments 2-5, wherein
m1 is 0, 1, 2, 3, 4, 5, 6, 7, 8, 9, or 10;
m2 is 1, 2, 3, 4, 5, 6, 7, 8, 9, or 10; and
m3 is 1, 2, 3, 4, 5, or 6.

### Embodiment 7

The compound of embodiment 2, wherein the linker LI corresponds to formula:

—(CH₂—CH₂—O)ₘ₂—CH₂CH₂-LX_{C}-;

—CH₂C(O)NH—(CH₂—CH₂)ₘ₂—CH₂CH₂-LX_{C}-;

—CH₂C(O)NH—(CH₂—CH₂—O)ₘ₂—CH₂-LX_{C}-;

—CH₂C(O)NH—(CH₂—CH₂—O)ₘ₂—CH₂CH₂-LX_{C}-; or

—CH₂C(O)NH—CH₂—(CH₂—CH₂—O)ₘ₂—CH₂CH₂CH₂-LX_{C}-; wherein —(CH₂—CH₂—O)ₘ₂ or —CH₂C(O)NH or is covalently bound to the PTC or PLM, and LX_{C}- is covalently bound to the PLM or PTC;
m2 is independently 1, 2, 3, 4, 5, or 6;
LX_{C} are each independently absent (a bond), —CH₂—, —O—, —S—, —S(O)—, —S(O)₂—, or —N(R²⁰)—;
wherein each R²⁰ is hydrogen or C₁-C₃ alkyl; and
wherein each —CH₂— in the linker is optionally substituted.

### Embodiment 8

The compound of embodiment 1, wherein the linker LI corresponds to formula

—(CH₂)ₘ₁-LX₁-(CH₂—CH₂-LX₂)ₘ₂—(CH₂)ₘ₃—C(LX₃)-, wherein:
—(CH₂)ₘ₁ is covalently bound to the PTC or PLM, and C(LX₃)- is covalently bound to the PLM or PTC;
each m1, m2, and m3 is independently 0, 1, 2, 3, 4, 5, 6, 7, 8, 9, or 10; and
   each LX₁, LX₂, and LX₃ is independently absent (a bond), —O—, —S—, —S(O)—, —S(O)₂—, or —N(R²⁰)—, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

### Embodiment 9

The compound of embodiment 8, wherein LX₁, LX₂, and LX₃ are —O—.

### Embodiment 10

The compound of embodiment 1, wherein the Linker corresponds to formula

—(CH₂)ₘ₁-LX_{B}-(CH₂)ₘ₂-LX_{C}-(CH₂)ₘ₃-LX_{D}-(CH₂)ₘ₄—C(O)—, wherein:
(CH₂)ₘ₁ is covalently bound to the PTC or PLM, and C(O) is covalently bound to the PLM or PTC;
each m1, and m2 is independently 0, 1, 2, 3, 4, 5, 6, 7, 8, 9, 10, 11, or 12;
m3 is independently 1, 2, 3, 4, 5, 6, 7, 8, 9, 10, 11, or 12;
m4 is 0, 1, 2, 3, 4, 5, 6, 7, 8, 9, 10, 11, or 12;
LX_{B}, LX_{C}, and LX_{D} are each independently absent (a bond), —CH₂—, —O—, —S—, —S(O)—, —S(O)₂, or —N(R²⁰)—;
wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

### Embodiment 11

The compound of embodiment 10, wherein the Linker corresponds to formula

—(CH₂)ₘ₁-LX_{B}-(CH₂)ₘ₂-LX_{C}-(CH₂)ₘ₃—O—(CH₂)ₘ₄—C(O)—, wherein:
(CH₂)ₘ₁ is covalently bound to the PTC, and C(O) is covalently bound to the PLM;
m1 is 0, 1, 2, or 3;
m2 is independently 0, 1, 2, 3, 4, or 5;
m3 is independently 1, 2, 3, 4, or 5;
m4 is 1, 2 or 3;
LX_{B} and LX_{C} are each independently absent (a bond), —O— or —N(R²⁰)—;
wherein each R²⁰ is independently selected from the group consisting of hydrogen, deuterium, and C₁-C₆ alkyl.

### Embodiment 12

The compound of any one of embodiments 2-11, wherein the sum of m1, m2, and m3 is less than or equal to 24.

### Embodiment 13

The compound of any one of embodiments 2-12, wherein the sum of m1, m2, and m3 is less than or equal to 12.

### Embodiment 14

The compound of embodiment 1, wherein the linker LI is a polyethylene glycol chain ranging in size from about 1 to about 12 ethylene glycol units, wherein each —CH₂— in the polyethylene glycol is optionally substituted.

### Embodiment 15

The compound of embodiment 14, wherein the linker LI is a polyethylene glycol chain ranging in size from about 2 to about 10 ethylene glycol units, wherein each —CH₂— in the polyethylene glycol is optionally substituted.

### Embodiment 16

The compound of embodiment 14, wherein the linker LI is a polyethylene glycol chain ranging in size from about 3 to about 5 ethylene glycol units, wherein each —CH₂— in the polyethylene glycol is optionally substituted.

### Embodiment 17

The compound of any one of embodiments 2-16, wherein the total number of atoms in a straight chain of LI connecting PTC and PLM is 20 or less.

### Embodiment 18

The compound of embodiment 1, wherein the linker LI corresponds to the formula:

-L_{I}-L_{II}(q)-,

wherein:
L_{I} is a bond or a chemical group coupled to at least one of a PLM, a PTC or a combination thereof,
L_{II} is a bond or a chemical group coupled to at least one of a PLM, a PTC, and q is an integer greater than or equal to 0;
wherein each L_{I} and L_{II} is independently selected from a bond, CR^{L1}R^{L2}, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, NR^{L3} SO₂NR^{L3}, SONR^{L3}CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁ cycloalkyl optionally substituted with 0-6 R^{L1} and/or R^{L2} groups, C₃₋₁₁ heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups;
wherein i is 1, 2, 3, 4, 5, 6, 7, 8, 9 or 10; and
wherein R^{L1}, R^{L2}, R^{L3}, R^{L4} and R^{L5} are, each independently, H, halo, —C₁₋₈ alkyl, —OC₁₋₈ alkyl, —SC₁₋₈ alkyl, —NHC₁₋₈ alkyl, —N(C₁₋₈ alkyl)₂, —C₃₋₁₁ cycloalkyl, aryl, heteroaryl, —C₃₋₁₁ heterocyclyl, —OC₁₋₈ cycloalkyl, —SC₁₋₈ cycloalkyl, —NHC₁₋₈ cycloalkyl, —N(C₁₋₈ cycloalkyl)₂, —N(C₁₋₈ cycloalkyl)(C₁₋₈ alkyl), —OH, —NH₂, —SH, —SO₂C₁₋₈ alkyl, —P(O)(OC₁₋₈ alkyl)(C₁₋₈ alkyl), —P(O)(OC₁₋₈ alkyl)₂, —C≡C—C₁₋₈ alkyl, —CCH, —CH═CH(C₁₋₈ alkyl), —C(C₁₋₈ alkyl)=CH(C₁₋₈ alkyl), —C(C₁₋₈ alkyl)=C(C₁₋₈ alkyl)₂, —Si(OH)₃, —Si(C₁₋₈ alkyl)₃, —Si(OH)(C₁₋₈ alkyl)₂, —C(═O)C₁₋₈ alkyl, —CO₂H, halogen, —CN, —CF₃, —CHF₂, —CH₂F, —NO₂, —SF₅, —SO₂NHC₁₋₈ alkyl, —SO₂N(C₁₋₈ alkyl)₂, —SONHC₁₋₈ alkyl, —SON(C₁₋₈ alkyl)₂, —CONHC₁₋₈ alkyl, —CON(C₁₋₈ alkyl)₂, —N(C₁₋₈ alkyl)CONH(C₁₋₈ alkyl), —N(C₁₋₈ alkyl)CON(C₁₋₈ alkyl)₂, —NHCONH(C₁₋₈ alkyl), —NHCON(C₁₋₈ alkyl)₂, —NHCONH₂, —N(C₁₋₈ alkyl)SO₂NH(C₁₋₈ alkyl), —N(C₁₋₈ alkyl)SO₂N(C₁₋₈ alkyl)₂, —NHSO₂NH(C₁₋₈ alkyl), —NHSO₂N(C₁₋₈ alkyl)₂, or —NHSO₂NH₂.

### Embodiment 19

The compound of embodiment 18, wherein q is selected from 1, 2, 3, 4, 5, 6, 7, 8, 9, 10, 11, 12, 13, 14, 15, 16, 17, 18, 19, 20, 21, 22, 23, or 24.

### Embodiment 20

The compound of embodiment 18 or 19, wherein L_{I} and L_{II} are independently selected from a bond, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, wherein i is 1, 2, 3, 4, 5, 6, 7, 8, 9 or 10, and at least one of L_{I} and L_{II} is not a bond.

### Embodiment 21

The compound of embodiment 1, wherein the linker LI is selected from Table L1, wherein LI is covalently bound to PLM by replacing a hydrogen from LI with a covalent bond to the PLM; and wherein LI is covalently bound to PTC by replacing a hydrogen from LI with a covalent bond the PTC.

### Embodiment 22

The compound of embodiment 1, wherein the linker LI is selected from Table L2.

### Embodiment 23

The compound of embodiment 1, wherein the linker LI is selected from Table L3.

### Embodiment 24

The compound of any one of embodiments 1-23, wherein the PLM is a von Hippel-Lindau (VHL) binding group, an E3 ligase substrate receptor cereblon (CRBN), a mouse double minute 2 homolog (MDM2), or an inhibitor of apoptosis (IAP).

### Embodiment 25

The compound of any one of embodiments 1-24, wherein the PLM is a von Hippel-Lindau (VHL) binding group.

### Embodiment 26

The compound of any one of embodiments 1-25, wherein the PLM has the formula (E3B):

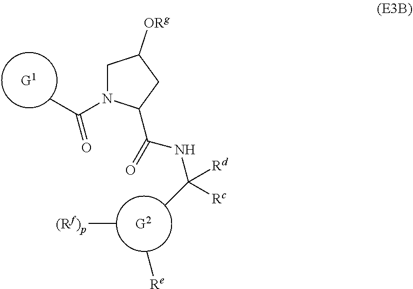

wherein, G¹ is optionally substituted aryl, optionally substituted heteroaryl, or —CR⁹R¹⁰R¹¹;
each R⁹ and R¹⁰ is independently hydrogen, optionally substituted alkyl, optionally substituted cycloalkyl, optionally substituted hydroxyalkyl, optionally substituted heteroaryl, or haloalkyl; or R⁹ and R¹⁰ and the carbon atom to which they are attached form an optionally substituted cycloalkyl;
R¹¹ is optionally substituted heterocyclic, optionally substituted alkoxy, optionally substituted heteroaryl, optionally substituted aryl, or —NR¹²R¹³,

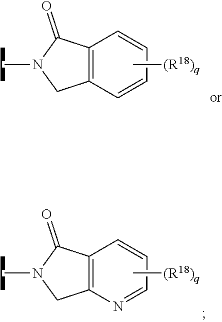

R¹² is H or optionally substituted alkyl;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} and R^{d} is each independently H, haloalkyl, or optionally substituted alkyl;
G² is a phenyl or a 5-10 membered heteroaryl,
R^{e} is H, halogen, CN, OH, NO₂, NR^{c}R^{d}, OR^{c}R, CONR^{c}R^{d}, NR^{c}COR^{d}, SO₂NR^{c}R^{d}, NR^{c}SO₂R^{d}, optionally substituted alkyl, optionally substituted haloalkyl, optionally substituted haloalkoxy; optionally substituted aryl; optionally substituted heteroaryl; optionally substituted cycloalkyl; optionally substituted cycloheteroalkyl;
each R^{f} is independently halo, optionally substituted alkyl, haloalkyl, hydroxy, optionally substituted alkoxy, or haloalkoxy;
R^{g} is H, C₁₋₆ alkyl, —C(O)R¹⁹; —C(O)OR¹⁹; or —C(O)NR¹⁹R¹⁹;
p is 0, 1, 2, 3, or 4;
each R¹⁸ is independently halo, optionally substituted alkoxy, cyano, optionally substituted alkyl, haloalkyl, haloalkoxy or a linker;
each R¹⁹ is independently H, optionally substituted alkyl, or optionally substituted aryl;
q is 0, 1, 2, 3, or 4; and
wherein any one of the hydrogen atoms in the PLM can be replaced to form a covalent bond to the LI.

### Embodiment 27

The compound of any one of embodiments 1-26, wherein the PLM has the formula (E3D):

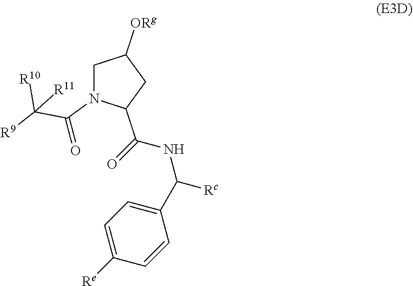

wherein, R⁹ is H;
R¹⁰ is C₁₋₆ alkyl;
R¹ is —NR¹²R¹³;
R¹² is H;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} is H, haloalkyl, methyl, ethyl, isopropyl, cyclopropyl, or C₁-C₆ alkyl (linear, branched, optionally substituted), each optionally substituted with 1 or more halo, hydroxyl, nitro, CN, C₁-C₆ alkyl (linear, branched, optionally substituted), or C₁-C₆ alkoxyl (linear, branched, optionally substituted); and
R^{e} is

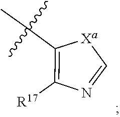

wherein R¹⁷ is H, halo, optionally substituted C₃₋₆cycloalkyl, optionally substituted C₁₋₆alkyl, optionally substituted C₁₋₆alkenyl, or C₁₋₆haloalkyl; and X^{a} is S or O;
R^{g} is H, C₁₋₆ alkyl, —C(O)R¹⁹; —C(O)OR¹⁹; or —C(O)NR¹⁹R¹⁹;
R¹⁹ is independently H, optionally substituted alkyl, or optionally substituted aryl; and wherein any one of the hydrogen atoms in the PLM can be replaced to form a covalent bond to the LI.

### Embodiment 28

The compound of embodiment 27, wherein the PLM is represented by formula (W-II):

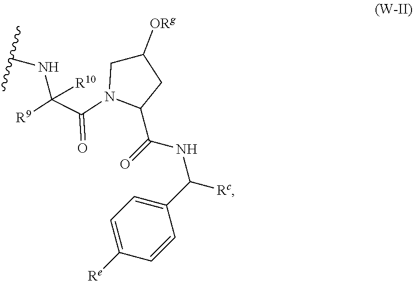

wherein the PLM is covalently bound to the LI via

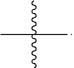

### Embodiment 29

The compound of embodiment 28, wherein the PLM is:

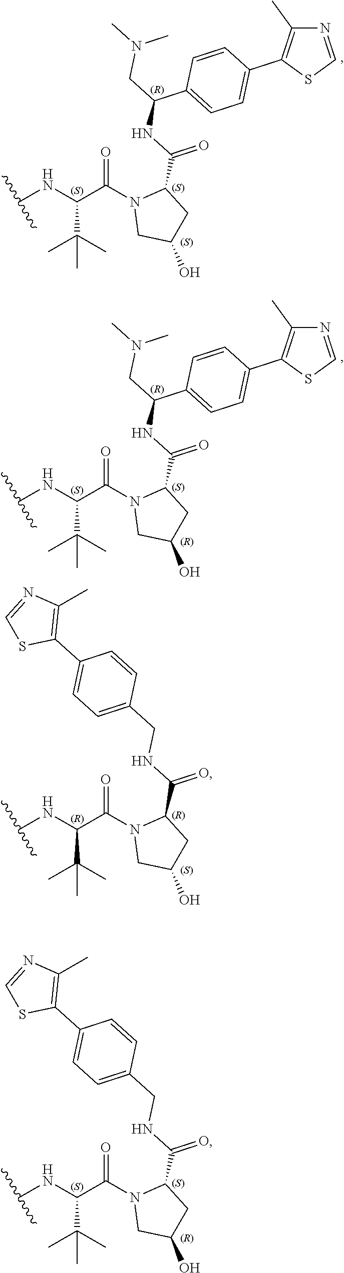

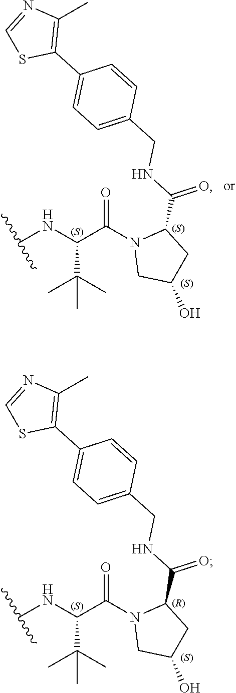

wherein the PLM is covalently bound to the LI via

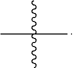

### Embodiment 30

The compound of any one of embodiments 1-25, wherein the PLM is represented by formula (W-IIIA):

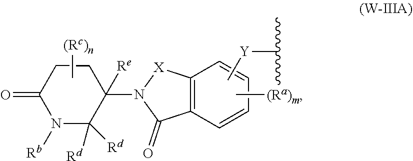

or an enantiomer, diastereomer, stereoisomer, or a pharmaceutically acceptable salt thereof, wherein:
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently halogen, OH, C₁₋₆ alkyl, or C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O), a C₃-C₆ carbocycle, or a 4- to 6-membered heterocycle comprising 1 or 2 heteroatoms selected from N or O;
R^{e} is H, deuterium, C₁₋₃ alkyl, F, or Cl;
m is 0, 1, 2 or 3;
n is 0, 1 or 2; and
wherein the PLM is covalently bound to the LI via

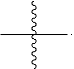

### Embodiment 31

The compound of embodiment 30, wherein the PLM is represented by formula (W-IIIB):

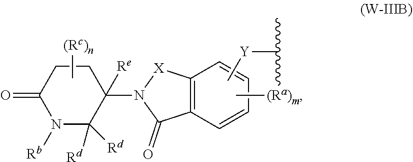

or an enantiomer, diastereomer, stereoisomer, or a pharmaceutically acceptable salt thereof, wherein:

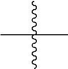

represent a bond to the LI;
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O) or a C₃-C₆ carbocycle;
R^{e} is H, deuterium, C₁₋₃ alkyl, F, or Cl;
m is 0, 1, 2 or 3;
n is 0, 1 or 2; and
wherein the PLM is covalently bound to the LI via

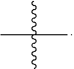

### Embodiment 32

The compound of embodiment 30 or 31, wherein X is —C(C₁₋₃ alkyl)₂.

### Embodiment 33

The compound of any one of embodiments 30-32, wherein the PLM is selected from the group consisting of:

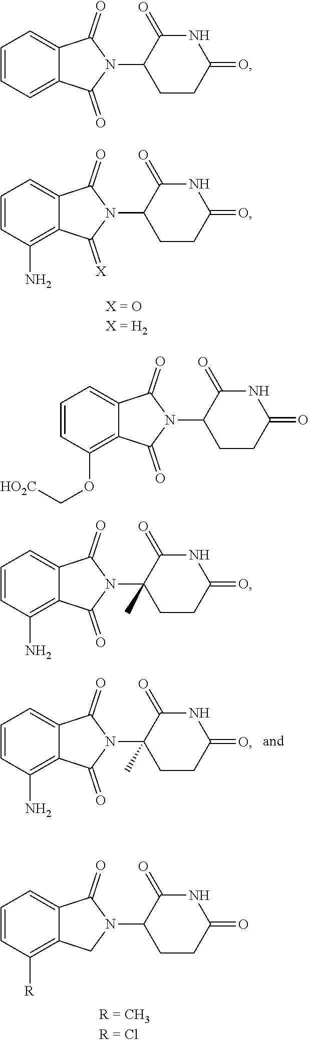

wherein any one of the hydrogen atoms in the PLM can be replaced to form a covalent bond to the LI.

### Embodiment 34

The compound of any one of embodiments 30-34, wherein the PLM is:

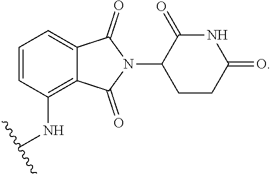 [SMILES: *Nc1cccc2c1C(=O)N(C1CCC(=O)NC1=O)C2=O]

### Embodiment 35

The compound of any one of embodiments 1-25, wherein the PLM is represented by:

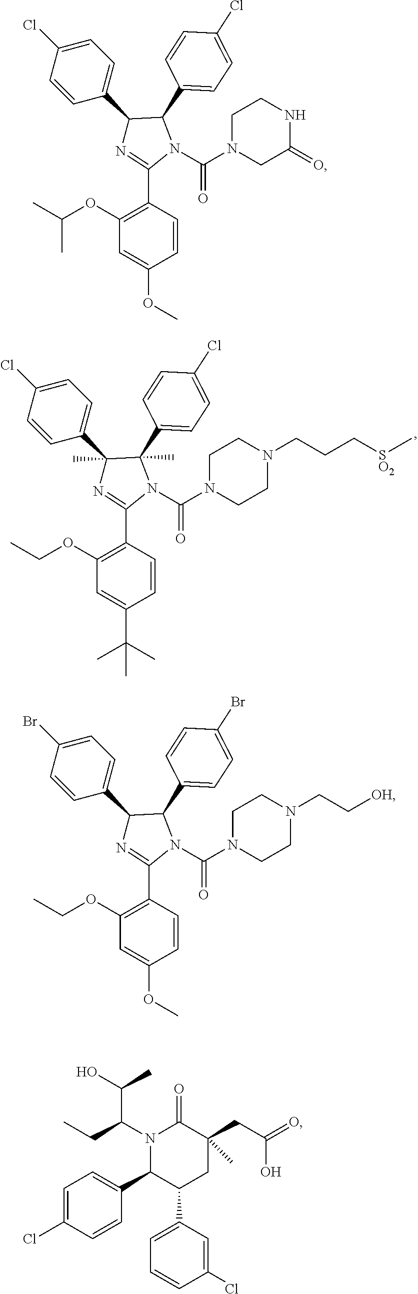

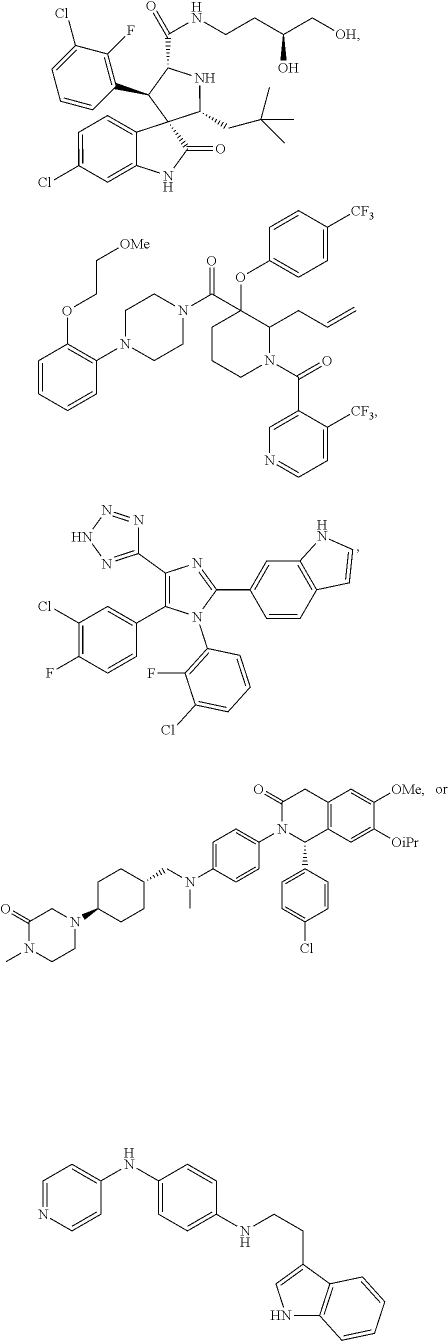 [SMILES: CC(C)(C)C[C@H]1N[C@@H](C(=O)NCC[C@H](O)CO)[C@H](c2cccc(Cl)c2F)[C@@]12C(=O)Nc1cc(Cl)ccc12 ; C=CCC1N(C(=O)c2cnccc2C(F)(F)F)CCCC1(Oc1ccc(C(F)(F)F)cc1)C(=O)N1CCN(c2ccccc2OCCOC)CC1 ; Fc1ccc(-c2c(-c3nn[nH]n3)nc(-c3ccc4cc[nH]c4c3)n2-c2cccc(Cl)c2F)cc1Cl ; COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2 ; c1ccc2c(CCNc3ccc(Nc4ccncc4)cc3)c[nH]c2c1]

and wherein any one of the hydrogen atoms in the PLM can be replaced to form a covalent bond to the LI.

### Embodiment 36

The compound of any one of embodiments 1-25, wherein the PLM is represented by

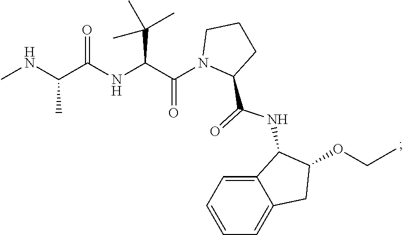 [SMILES: CCO[C@@H]1Cc2ccccc2[C@@H]1NC(=O)[C@@H]1CCCN1C(=O)[C@@H](NC(=O)[C@H](C)NC)C(C)(C)C]

wherein any one of the hydrogen atoms in the PLM can be replaced to form a covalent bond to the LI.

### Embodiment 37

The compound of any one of embodiments 1-25, wherein the PLM is

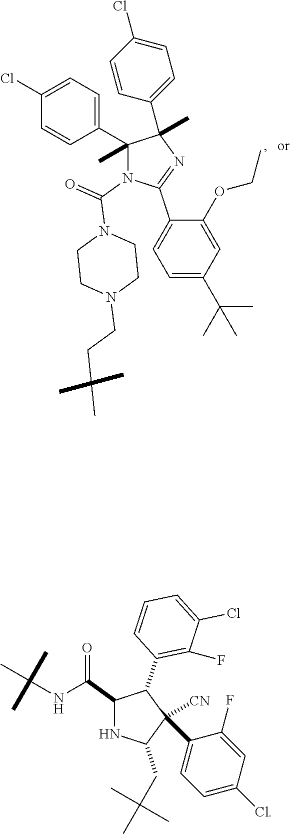

### Embodiment 38

The compound of any one of embodiments 1-37, wherein the PTC has the structure of formula (IVA):

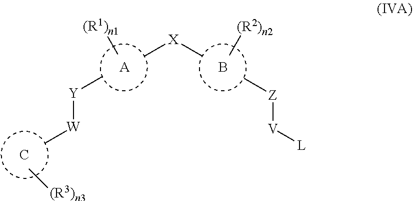

or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y and Z are each independently a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, C₁-C₃ alkyl, C₂-C₃ alkenyl, or C₂-C₃ alkynyl;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

### Embodiment 39

The compound of embodiment 38, wherein C is 5- to 10-membered heteroaryl or aryl.

### Embodiment 40

The compound of embodiment 38 or 39, wherein C is 5- to 7-membered heteroaryl comprising 1, 2, or 3 heteroatoms selected from O, S, or N as a ring member.

### Embodiment 41

The compound of any one of embodiments 38-40, wherein C, which is substituted with (R³)n3, is pyrazole, imidazole, oxazole, oxadiazole, oxazolone, isoxazole, thiazole, pyridyl, pyrazine, furan or pyrimidyl.

### Embodiment 42

The compound of any one of embodiments 38-40, wherein C, which is substituted with (R³)n3, is selected from the group consisting of:

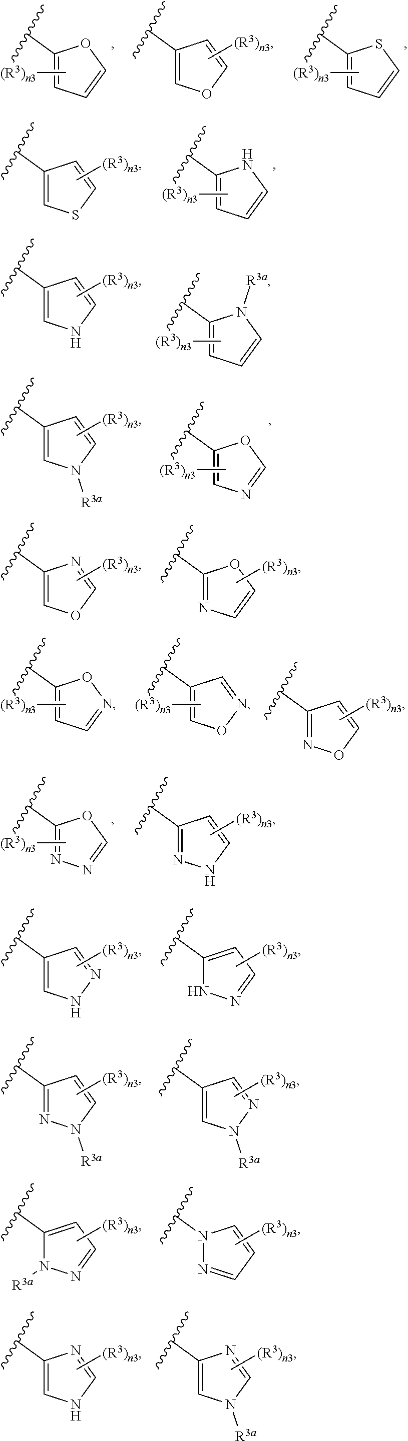

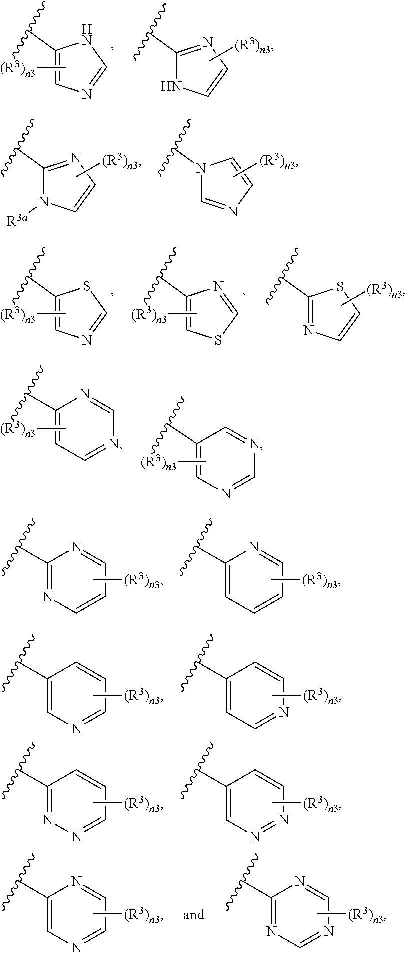

wherein R^{3a} is C₁-C₃ alkyl.

### Embodiment 43

The compound of any one of embodiments 38-42, wherein R¹ and R² are each independently Cl, —CN, —CF₃, —OH, methyl, methoxy, or —CONH₂.

### Embodiment 44

The compound of any one of embodiments 38-43, wherein:
A and B are phenyl;
X is —(CR⁵R⁶)ₜ—;
Y and Z are each —O—;
V is —CH₂— or —CH₂CH₂—;
L is halogen;
R¹ and R² are each independently hydrogen, halogen, —CN, —CF₃, —OH, or optionally substituted C₁-C₆ alkyl;
R⁵ and R⁶ are each independently hydrogen, halogen, —OH, or C₁-C₃ alkyl; and
R¹⁶ is hydrogen, C₁-C₃ alkyl, C₂-C₃ alkenyl, or C₂-C₃ alkynyl.

### Embodiment 45

The compound of embodiment 44, wherein:
R⁵ and R⁶ are each independently hydrogen, or C₁-C₃ alkyl;
W is —CH₂— or —C(CH₃)H—;
V is —CH₂CH₂—; and
R¹ and R² are each independently hydrogen, halogen, or —CN.

### Embodiment 46

The compound of embodiment 38, wherein the PTC has the structure of formula (A-I)

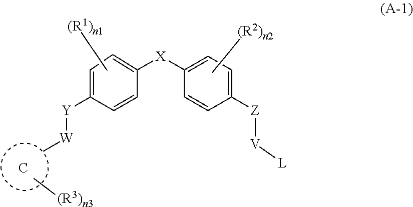

or a pharmaceutically acceptable salt, tautomer, stereoisomer or prodrug thereof, wherein:
C is a 5- to 7-membered monocyclic heteroaryl comprising 1, 2, or 3 heteroatoms selected from O, S, or N as a ring member;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, methyl, or —CONH₂;
R³ is selected from —CN, C₁-C₃ alkoxy, —CF₃, C₁-C₃ alkyl, C₂-C₃ alkenyl, C₂-C₃ alkynyl, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

### Embodiment 47

The compound of any one of embodiments 38-46, wherein: at least one R³ is selected from the group consisting of —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), and —N(CH₃)COO(C₁-C₃ alkyl).

### Embodiment 48

The compound of embodiment 46, wherein:
X is a bond or —(CR⁵R⁶)ₜ;
W is a bond, —CH₂—, or —C(CH₃)H—;
Y is —O—;
Z is —O—;
V is —CH₂— or —CH₂CH₂—; and
L is halogen.

### Embodiment 49

The compound of embodiment 1, wherein the PTC has the structure of formula (G-II):

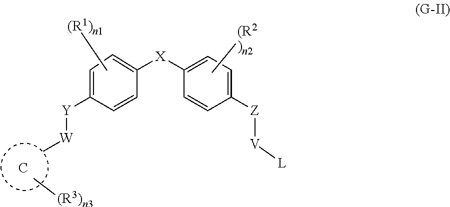

or a pharmaceutically acceptable salt, tautomer, stereoisomer or prodrug thereof, wherein:
C is

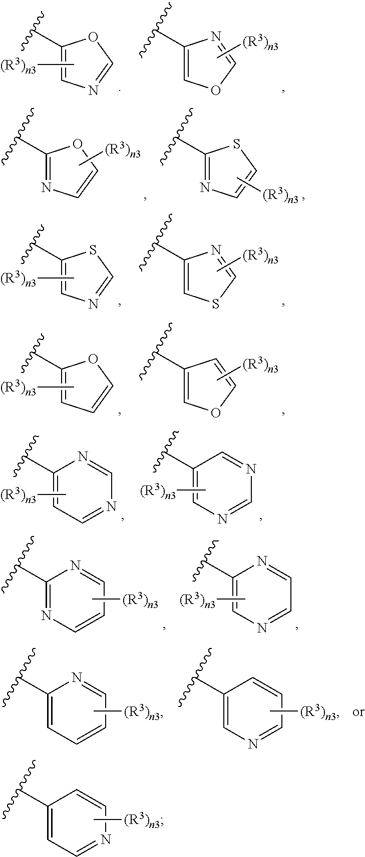

X is —(CR⁵R⁶)ₜ—;
Y is —O—;
Z is —O—;
W is —CH₂— or —C(CH₃)H—;
V is —CH₂CH₂—;
L is halogen;
R¹ and R² are each independently Cl or —CN;
at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or methyl;
n1 and n2 are each independently 0, 1, or 2;
n3 is 1 or 2;
t is 1; and
   wherein one atom or one chemical group in the PTC is replaced to form a covalent bond to the LI.

### Embodiment 50

The compound of embodiment 49, wherein: at least one R³ is selected from the group consisting of —NHSO₂CH₃, —NHSO₂CH₂CH₃, or —SO₂CH₃ and the other R³, if present, is selected from —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), and —N(CH₃)COO(C₁-C₃ alkyl).

### Embodiment 51

The compound of any one of embodiments 1-50 wherein an atom in L is replaced with a covalent bond to the LI.

### Embodiment 52

The compound of embodiment 51, wherein a halogen is replaced with a covalent bond to the LI

### Embodiment 53

The compound of any one of embodiments 1-50, wherein an atom in ring C, R¹, or R³, is replaced with a covalent bond to the LI.

### Embodiment 54

The compound of embodiment 53, wherein a hydrogen atom is replaced with a covalent bond to the LI

### Embodiment 55

The compound of embodiment 1, wherein the PTC is selected from Table A or a pharmaceutically acceptable salt, tautomer, stereoisomer or prodrug thereof, wherein one atom or one chemical group in the PTC is replaced to form a covalent bond to the LI.

### Embodiment 56

The compound of embodiment 55, wherein the PTC is selected from the group consisting of:

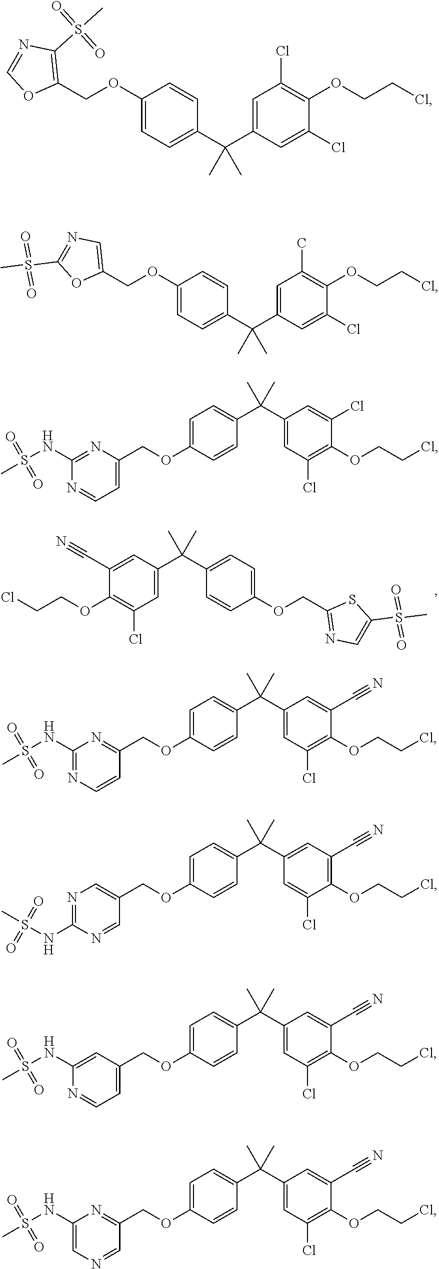

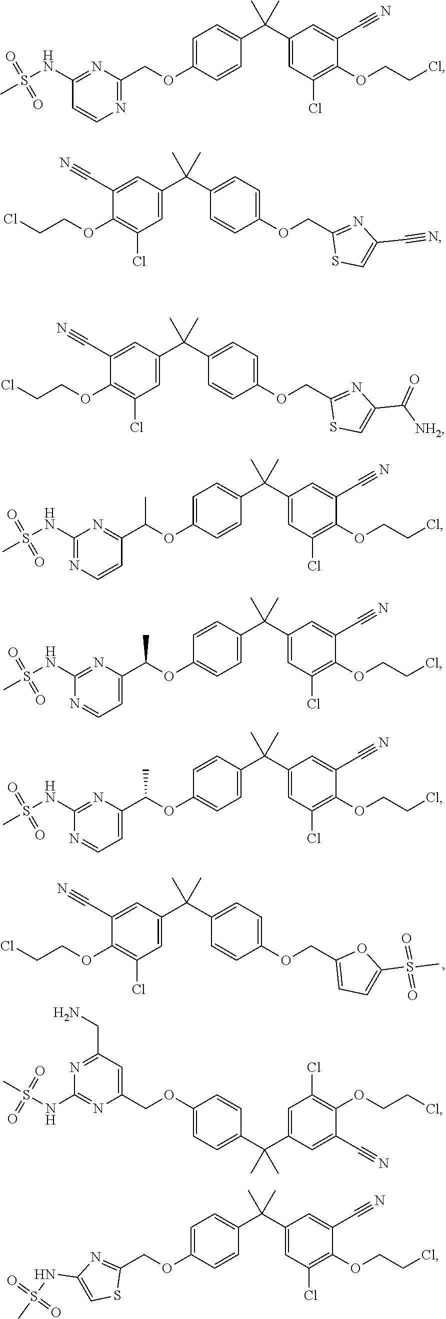

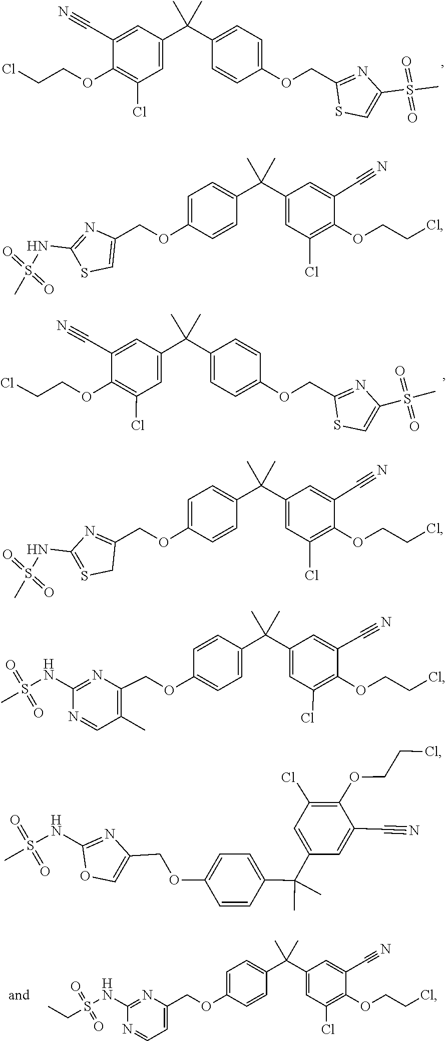

or a pharmaceutically acceptable salt, tautomer, stereoisomer or prodrug thereof; and wherein one atom or one chemical group in the PTC is replaced to form a covalent bond to the LI.

### Embodiment 57

The compound of embodiment 55 or 56, wherein a Cl atom is replaced with a covalent bond to the LI.

### Embodiment 58

The compound of embodiment 55 or 56, wherein a hydrogen atom is replaced with a covalent bond to the LI.

### Embodiment 59

The compound of any one of the preceding embodiments, wherein the PTC is selected from:

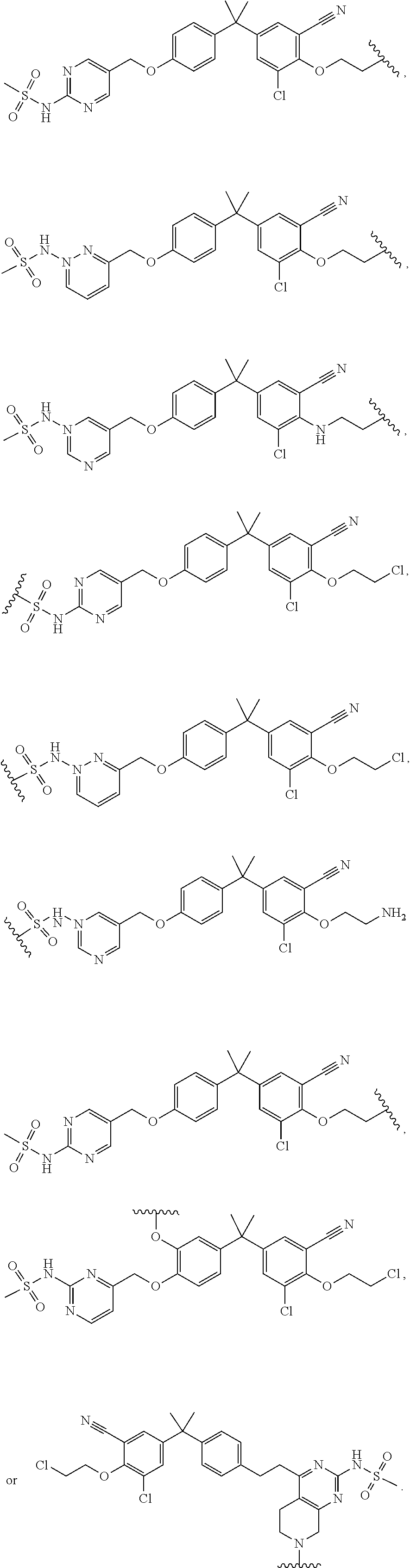

### Embodiment 60

The compound of any one of embodiments 1-59, wherein the compound is a compound of formula (W-IV):

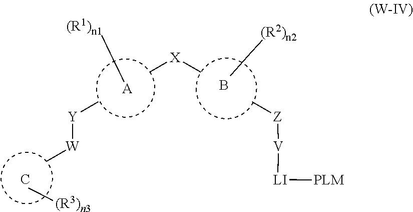

or a pharmaceutically acceptable salt thereof.

### Embodiment 61

The compound of embodiment 60, wherein the compound is a compound of formula (W-IVA)

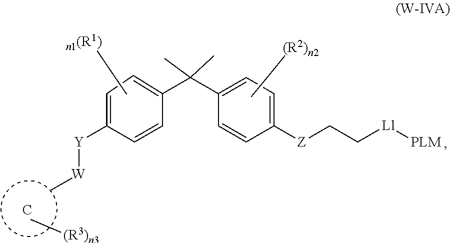

or a pharmaceutically acceptable salt thereof.

### Embodiment 62

The compound of any one of embodiments 1-59, wherein the compound is a compound of formula (W-V):

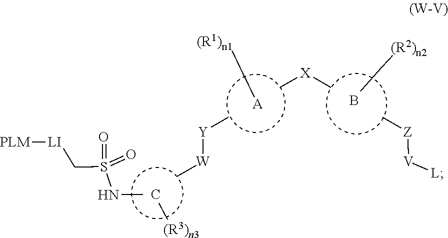

or a pharmaceutically acceptable salt thereof.

### Embodiment 63

The compound of embodiment 62, wherein the compound is a compound of formula (W-VA):

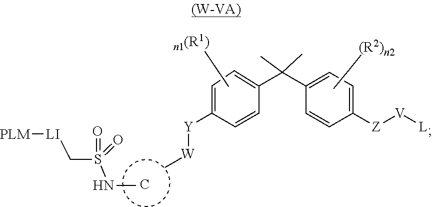

or a pharmaceutically acceptable salt thereof.

### Embodiment 64

The compound of any one of embodiments 1-59, wherein the compound is a compound of formula (W-VI):

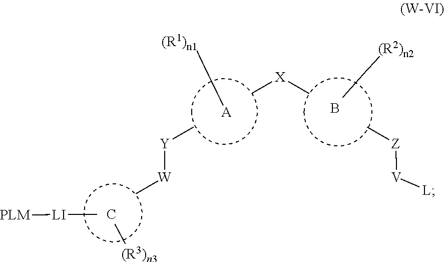

or a pharmaceutically acceptable salt thereof.

### Embodiment 65

The compound of embodiment 64, wherein the compound is a compound of formula (W-VIA):

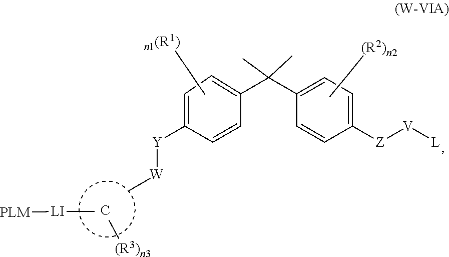

or a pharmaceutically acceptable salt thereof.

### Embodiment 66

The compound of any one of embodiments 1-59, wherein the compound is a compound of formula (W-VII):

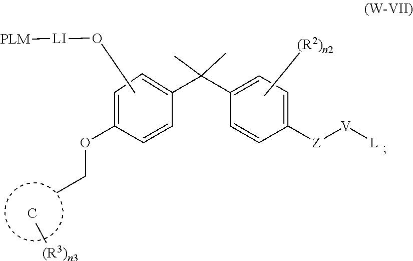

or a pharmaceutically acceptable salt thereof.

### Embodiment 67

A compound selected from Table P. or a pharmaceutically acceptable salt thereof.

### Embodiment 68

A pharmaceutical composition comprising a compound of any one of embodiments 1-67 and a pharmaceutically acceptable carrier.

### Embodiment 69

The pharmaceutical composition of embodiment 68, further comprising one or more additional therapeutic agents.

### Embodiment 70

The pharmaceutical composition of embodiment 68, wherein the one or more additional therapeutic agents is for treating prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

### Embodiment 71

The pharmaceutical composition of embodiment 68, wherein the one or more additional therapeutic agents is a poly (ADP-ribose) polymerase (PARP) inhibitor including but not limited to olaparib, niraparib, rucaparib, talazoparib; an androgen receptor ligand binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide; a 5-alpha reductase inhibitor including but not limited to finasteride, dutasteride, turosteride, bexlosteride, izonsteride, FCE 28260, SKF105,111; a vascular endothelial growth-factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an N-terminal domain inhibitor including but not limited to a sintokamide; an alpha-particle emitting radioactive therapeutic agent including but not limited to radium 233 or a salt thereof; niclosamide; or related compounds thereof, a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromitase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; CDK4/6 inhibitors including palbociclib, abemaciclib, ribociclib; HER2 receptor antagonist including but not limited to trastuzumab, neratinib; or a mammalian target of rapamycin (mTOR) inhibitor including but not limited to everolimus, temsirolimus.

### Embodiment 72

A method for modulating androgen receptor activity, comprising administering a compound of any one of embodiments 1-67, to a subject in need thereof.

### Embodiment 73

The method of embodiment 71, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

### Embodiment 74

A method for treating cancer, comprising administering a compound of any one of embodiments 1-67, to a subject in need thereof.

### Embodiment 75

A compound of formula (Q):

PLM-LI-PTC (Q);

or a pharmaceutically acceptable salt thereof, wherein:
PLM is a E3 ligase binding group,
LI is a linker, and
PTC is an androgen receptor modulator.

### Embodiment 76

The compound of embodiment 75, wherein the linker LI corresponds to formula

-LX_{A}-(CH₂)ₘ₁—(CH₂—CH₂-LX_{B})ₘ₂—(CH₂)ₘ₃-LX_{C}-, wherein:
-LX_{A} is covalently bound to the PTC or PLM, and LX_{C}- is covalently bound to the PLM or PTC;
each m1 and m2 is independently 0, 1, 2, 3, 4, 5, 6, 7, 8, 9, 10, 11, or 12;
m3 is independently 1, 2, 3, 4, 5, 6, 7, 8, 9, 10, 11, or 12;
LX_{A} is absent (a bond), —CH₂C(O)NR²⁰—, or —NR²⁰C(O)CH₂—;
LX_{B} and LX_{C} are each independently absent (a bond), —CH₂—, —O—, —S—, —S(O)—, —S(O)₂—, or —N(R²⁰)—;
wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

### Embodiment 77

The compound of embodiment 76, wherein LX_{A} is absent (a bond), —CH₂C(O)NR²⁰—, or —NR²⁰C(O)CH₂—; wherein R²⁰ is hydrogen or C₁-C₃ alkyl.

### Embodiment 78

The compound of embodiment 76 or 77, wherein LX_{B} is absent (a bond), —CH₂—, —O— or —N(R²⁰)—; wherein R²⁰ is hydrogen or C₁-C₃ alkyl.

### Embodiment 79

The compound of any one of embodiments 76-78, wherein LX_{C} is absent (a bond), —CH₂—, —O—, or —NH—.

### Embodiment 80

The compound of any one of embodiments 76-79, wherein
m1 is 0, 1, 2, 3, 4, 5, 6, 7, 8, 9, or 10;
m2 is 1, 2, 3, 4, 5, 6, 7, 8, 9, or 10; and
m3 is 1, 2, 3, 4, 5, or 6.

### Embodiment 81

The compound of any one of embodiments 76-80, wherein sum of m1, m2, and m3 is less than or equal to 24.

### Embodiment 82

The compound of any one of embodiments 76-80, wherein sum of m1, m2, and m3 is less than or equal to 12.

### Embodiment 83

The compound of embodiment 76, wherein the linker LI corresponds to formula:

—(CH₂—CH₂—O)ₘ₂—CH₂CH₂-LX_{C}-;

—CH₂C(O)NH—(CH₂—CH₂)ₘ₂—CH₂CH₂-LX_{C}-;

—CH₂C(O)NH—(CH₂—CH₂—O)ₘ₂—CH₂-LX_{C}-;

—CH₂C(O)NH—(CH₂—CH₂—O)ₘ₂—CH₂CH₂-LX_{C}-; or

—CH₂C(O)NH—CH₂—(CH₂—CH₂—O)ₘ₂—CH₂CH₂CH₂-LX_{C}-; wherein —(CH₂—CH₂—O)ₘ₂ or —CH₂C(O)NH or is covalently bound to the PTC or PLM, and LX_{C}- is covalently bound to the PLM or PTC;
m2 is independently 1, 2, 3, 4, 5, or 6;
LX_{C} are each independently absent (a bond), —CH₂—, —O—, —S—, —S(O)—, —S(O)₂—, or —N(R²⁰)—;
wherein each R²⁰ is hydrogen or C₁-C₃ alkyl; and
wherein each —CH₂— in the linker is optionally substituted.

### Embodiment 84

The compound of any one of embodiments 76-83, wherein the total number of atoms in a straight chain of LI connecting PTC and PLM is 20 or less.

### Embodiment 85

The compound of embodiment 75, wherein the linker LI corresponds to formula

—(CH₂)ₘ₁-LX₁-(CH₂—CH₂-LX₂)ₘ₂—(CH₂)ₘ₃—C(LX₃)-, wherein:
—(CH₂)ₘ₁ is covalently bound to the PTC or PLM, and C(LX₃)- is covalently bound to the PLM or PTC;
each m1, m2, and m3 is independently 0, 1, 2, 3, 4, 5, 6, 7, 8, 9, or 10; and
   each LX₁, LX₂, and LX₃ is independently absent (a bond), —O—, —S—, —S(O)—, —S(O)₂—, or —N(R²⁰)—, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

### Embodiment 86

The compound of embodiment 85, wherein LX₁, LX₂, and LX₃ are —O—.

### Embodiment 87

The compound of embodiment 85, wherein the sum of m1, m2, and m3 is less than or equal to 24.

### Embodiment 88

The compound of embodiment 75, wherein the linker LI is a polyethylene glycol chain ranging in size from about 1 to about 12 ethylene glycol units, wherein each —CH₂— in the the polyethylene glycol is optionally substituted.

### Embodiment 89

The compound of embodiment 88, wherein the linker LI is a polyethylene glycol chain ranging in size from about 2 to about 10 ethylene glycol units, wherein each —CH₂— in the the polyethylene glycol is optionally substituted.

### Embodiment 90

The compound of embodiment 88, wherein the linker LI is a polyethylene glycol chain ranging in size from about 3 to about 5 ethylene glycol units, wherein each —CH₂— in the the polyethylene glycol is optionally substituted.

### Embodiment 91

The compound of embodiment 75, wherein the linker LI corresponds to the formula:

-L_{I}-L_{II}(q)-,

wherein:
L_{I} is a bond or a chemical group coupled to at least one of a PLM, a PTC or a combination thereof,
L_{II} is a bond or a chemical group coupled to at least one of a PLM, a PTC, and q is an integer greater than or equal to 0;
wherein each LI and L_{II} is independently selected from a bond, CR^{L1}R^{L2}, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, NR^{L3} SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁ cycloalkyl optionally substituted with 0-6 R^{L1} and/or R^{L2} groups, C₃₋₁₁ heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups;
wherein i is 1, 2, 3, 4, 5, 6, 7, 8, 9 or 10; and
wherein R^{L1}, R^{L2}, R^{L3}, R^{L4} and R^{L5} are, each independently, H, halo, —C₁₋₈ alkyl, —OC₁₋₈ alkyl, —SC₁₋₈ alkyl, —NHC₁₋₈ alkyl, —N(C₁₋₈ alkyl)₂, —C₃₋₁₁ cycloalkyl, aryl, heteroaryl, —C₃₋₁₁ heterocyclyl, —OC₁₋₈ cycloalkyl, —SC₁₋₈ cycloalkyl, —NHC₁₋₈ cycloalkyl, —N(C₁₋₈ cycloalkyl)₂, —N(C₁₋₈ cycloalkyl)(C₁₋₈ alkyl), —OH, —NH₂, —SH, —SO₂C₁₋₈ alkyl, —P(O)(OC₁₋₈ alkyl)(C₁₋₈ alkyl), —P(O)(OC₁₋₈ alkyl)₂, —C≡C—C₁₋₈ alkyl, —CCH, —CH═CH(C₁₋₈ alkyl), —C(C₁₋₈ alkyl)=CH(C₁₋₈ alkyl), —C(C₁₋₈ alkyl)=C(C₁₋₈ alkyl)₂, —Si(OH)₃, —Si(C₁₋₈ alkyl)₃, —Si(OH)(C₁₋₈ alkyl)₂, —C(═O)C₁₋₈ alkyl, —CO₂H, halogen, —CN, —CF₃, —CHF₂, —CH₂F, —NO₂, —SF₅, —SO₂NHC₁₋₈ alkyl, —SO₂N(C₁₋₈ alkyl)₂, —SONHC₁₋₈ alkyl, —SON(C₁₋₈ alkyl)₂, —CONHC₁₋₈ alkyl, —CON(C₁₋₈ alkyl)₂, —N(C₁₋₈ alkyl)CONH(C₁₋₈ alkyl), —N(C₁₋₈ alkyl)CON(C₁₋₈ alkyl)₂, —NHCONH(C₁₋₈ alkyl), —NHCON(C₁₋₈ alkyl)₂, —NHCONH₂, —N(C₁₋₈ alkyl)SO₂NH(C₁₋₈ alkyl), —N(C₁₋₈ alkyl)SO₂N(C₁₋₈ alkyl)₂, —NHSO₂NH(C₁₋₈ alkyl), —NHSO₂N(C₁₋₈ alkyl)₂, or —NHSO₂NH₂.

### Embodiment 92

The compound of embodiment 91, wherein q is selected from 1, 2, 3, 4, 5, 6, 7, 8, 9, 10, 11, 12, 13, 14, 15, 16, 17, 18, 19, 20, 21, 22, 23, or 24.

### Embodiment 93

The compound of embodiment 91 or 92, wherein L_{I} and L_{II} are independently selected from a bond, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, wherein i is 1, 2, 3, 4, 5, 6, 7, 8, 9 or 10, and at least one of L_{I} and L_{II} is not a bond.

### Embodiment 94

The compound of embodiment 75, wherein the linker LI is selected from Table L1, wherein LI is covalently bound to PLM by replacing a hydrogen from LI with a covalent bond and wherein LI is covalently bound to PTC by replacing a hydrogen from LI with a covalent bond.

### Embodiment 95

The compound of embodiment 75, wherein the linker LI is selected from Table L2.

### Embodiment 96

The compound of any one of embodiments 75-95, wherein the PLM is a von Hippel-Lindau (VHL) binding group, an E3 ligase substrate receptor cereblon (CRBN), a mouse double minute 2 homolog (MDM2), or an inhibitor of apoptosis (IAP).

### Embodiment 97

The compound of any one of embodiments 75-95, wherein the PLM is a von Hippel-Lindau (VHL) binding group.

### Embodiment 98

The compound of any one of embodiments 75-97, wherein the PLM has the formula (E3B):

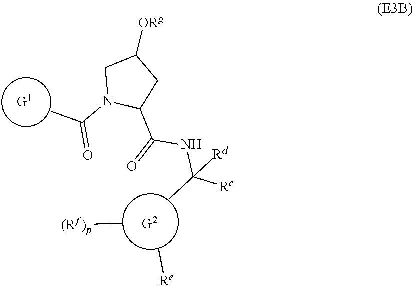

wherein, G¹ is optionally substituted aryl, optionally substituted heteroaryl, or —CR⁹R¹⁰R¹¹;
each R⁹ and R¹⁰ is independently hydrogen, optionally substituted alkyl, optionally substituted cycloalkyl, optionally substituted hydroxyalkyl, optionally substituted heteroaryl, or haloalkyl; or R⁹ and R¹⁰ and the carbon atom to which they are attached form an optionally substituted cycloalkyl;
R¹¹ is optionally substituted heterocyclic, optionally substituted alkoxy, optionally substituted heteroaryl, optionally substituted aryl, or —NR¹²R¹³,

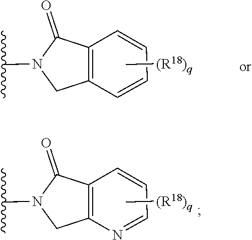

R¹² is H or optionally substituted alkyl;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} and R^{d} is each independently H, haloalkyl, or optionally substituted alkyl;
G² is a phenyl or a 5-10 membered heteroaryl,
R^{e} is H, halogen, CN, OH, NO₂, NR^{c}R^{d}, OR^{c}R, CONR^{c}R^{d}, NR^{c}COR^{d}, SO₂NR^{c}R^{d}, NR^{c}SO₂R^{d}, optionally substituted alkyl, optionally substituted haloalkyl, optionally substituted haloalkoxy; optionally substituted aryl; optionally substituted heteroaryl; optionally substituted cycloalkyl; optionally substituted cycloheteroalkyl;
each R^{f} is independently halo, optionally substituted alkyl, haloalkyl, hydroxy, optionally substituted alkoxy, or haloalkoxy;
R^{g} is H, C₁₋₆ alkyl, —C(O)R¹⁹; —C(O)OR¹⁹; or —C(O)NR¹⁹R¹⁹;
p is 0, 1, 2, 3, or 4;
each R¹⁸ is independently halo, optionally substituted alkoxy, cyano, optionally substituted alkyl, haloalkyl, haloalkoxy or a linker;
each R¹⁹ is independently H, optionally substituted alkyl, or optionally substituted aryl;
q is 0, 1, 2, 3, or 4; and
wherein any one of the hydrogen atom in the PLM can be replaced to form a covalent bond to LI.

### Embodiment 99

The compound of any one of embodiments 75-97, wherein the PLM has the formula (E3D):

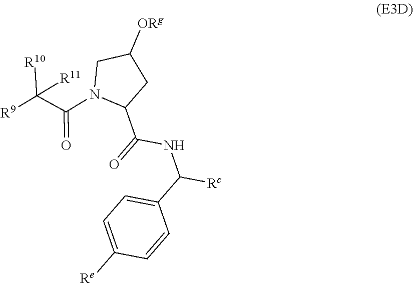

wherein, R⁹ is H;
R¹⁰ is C₁₋₆ alkyl;
R¹¹ is —NR¹²R¹³;
R¹² is H;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} is H, haloalkyl, methyl, ethyl, isopropyl, cyclopropyl, or C₁-C₆ alkyl (linear, branched, optionally substituted), each optionally substituted with 1 or more halo, hydroxyl, nitro, CN, C₁-C₆ alkyl (linear, branched, optionally substituted), or C₁-C₆ alkoxyl (linear, branched, optionally substituted); and
R^{e} is

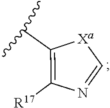

wherein R¹⁷ is H, halo, optionally substituted C₃₋₆cycloalkyl, optionally substituted C₁₋₆alkyl, optionally substituted C₁₋₆alkenyl, or C₁₋₆haloalkyl; and X^{a} is S or O;
R^{g} is H, C₁₋₆ alkyl, —C(O)R¹⁹; —C(O)OR¹⁹; or —C(O)NR¹⁹R¹⁹;
R¹⁹ is independently H, optionally substituted alkyl, or optionally substituted aryl; and
wherein any one of the hydrogen atom in the PLM can be replaced to form a covalent bond to LI.

### Embodiment 100

The compound of embodiment 99, wherein the PLM has the following connectivity to the linker LI:

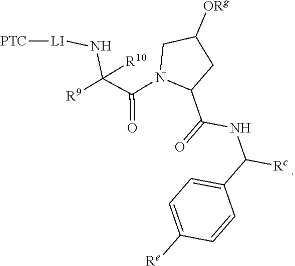

### Embodiment 101

The compound of any one of embodiments 75-97, wherein the PLM has the formula (E3E):

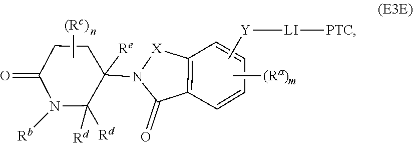

or an enantiomer, diastereomer, stereoisomer, or a pharmaceutically acceptable salt thereof, wherein:
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR or;
X is —C(O)— or —C(R^{b})₂—;
each R^{a} is independently halogen, OH, C₁₋₆ alkyl, or C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O), a C₃-C₆ carbocycle, or a 4- to 6-membered heterocycle comprising 1 or 2 heteroatoms selected from N or O;
R^{e} is H, deuterium, C₁₋₃ alkyl, F, or Cl;
m is 0, 1, 2 or 3;
n is 0, 1 or 2; and
wherein any one of the hydrogen atom in the PLM can be replaced to form a covalent bond to LI.

### Embodiment 102

The compound of any one of embodiments 75-97, wherein the PLM has the formula (E3F):

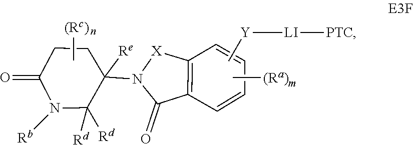

or an enantiomer, diastereomer, stereoisomer, or a pharmaceutically acceptable salt thereof, wherein:
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O) or a C₃-C₆ carbocycle;
R^{e} is H, deuterium, C₁₋₃ alkyl, F, or Cl;
m is 0, 1, 2 or 3;
n is 0, 1 or 2; and
wherein any one of the hydrogen atom in the PLM can be replaced to form a covalent bond to LI.

### Embodiment 103

The compound of embodiment 101 or 102, wherein X is —C(C₁₋₃ alkyl)₂.

### Embodiment 104

The compound of any one of embodiments 75-97, wherein the PLM is selected from

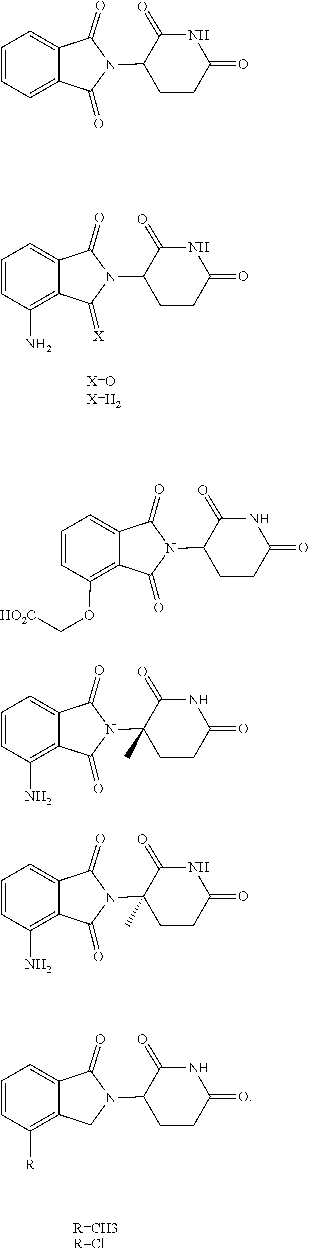

### Embodiment 105

The compound of any one of embodiments 75-97, wherein the PLM is selected from

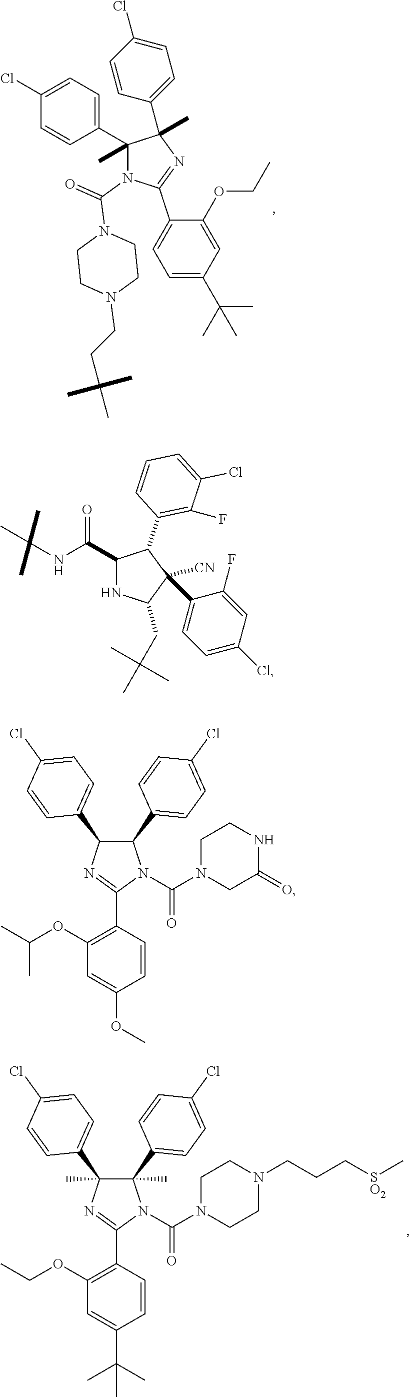

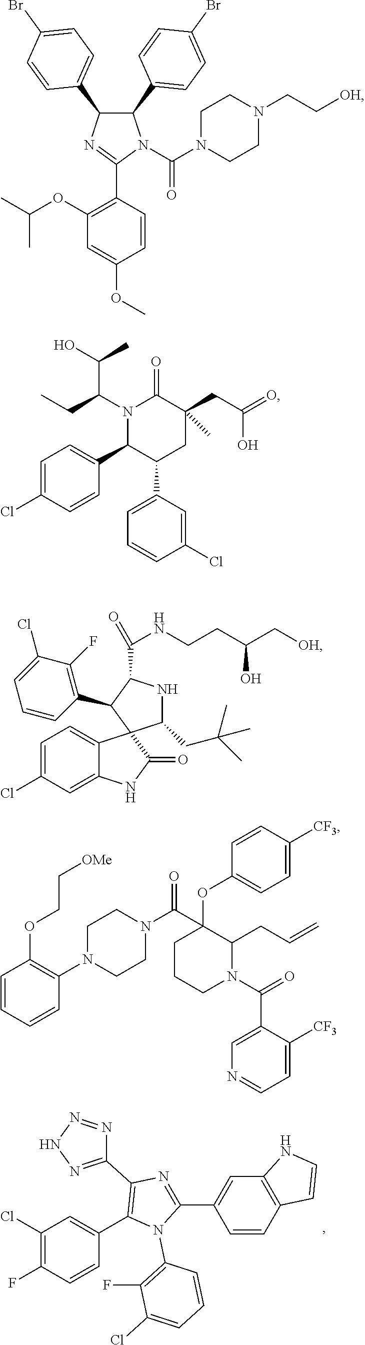

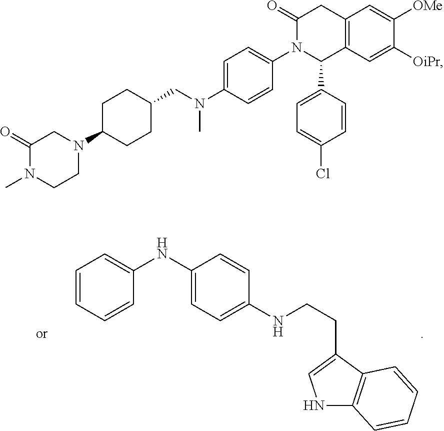 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2 ; c1ccc(Nc2ccc(NCCc3c[nH]c4ccccc34)cc2)cc1]

### Embodiment 106

The compound of any one of embodiments 75-95 wherein the PLM is

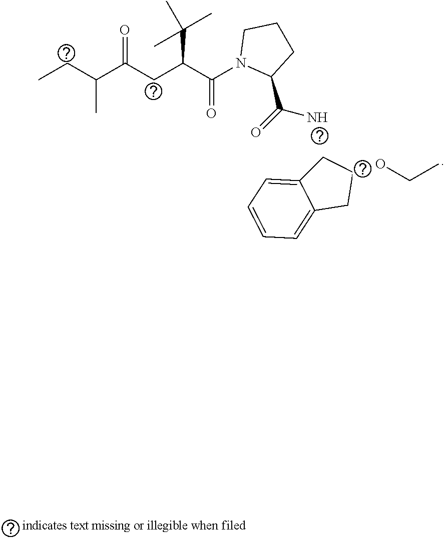

### Embodiment 107

The compound of any one of embodiments 75-106, wherein the PTC has the formula

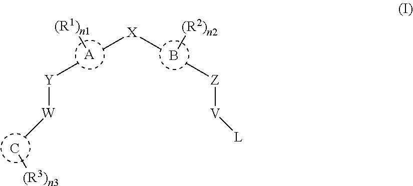

or a pharmaceutically acceptable salt, tautomer, stereoisomer or prodrug thereof, wherein:
A and B are each independently aryl or heteroaryl;
C is a 3- to 10-membered ring;
X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;
Y and Z are each independently a bond, —(CR⁸R⁹)ₘ—, —O—, —C(═O)—, —S—, —S(═O)—, —SO₂—, or —NR⁷—;
W and V are each independently a bond, —(CR^{8a}R^{9a})ₘ—, —C(═O)—, —N(R⁷)CO—, —CONR⁷—, or —NSO₂R⁷—;
L is hydrogen, halogen, optionally substituted alkyl sulfonate, optionally substituted aryl sulfonate, —CF₂R¹⁰, —CF₃, —CN, —OR¹⁰; —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —COOH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COOR¹⁶, —NR¹⁴COR¹⁶, —NR¹⁴CONR¹⁴R¹⁵, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R and R⁶ taken together form an optionally substituted carbocyclyl or optionally substituted heterocyclyl;
R⁸ and R⁹ are each independently hydrogen, halogen, or C₁-C₃ alkyl;
R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{8b} taken together form an optionally substituted carbocyclyl or optionally substituted heterocyclyl;
R⁷, R¹⁰ and R¹⁶ are each independently hydrogen, optionally substituted C₁-C₆ alkyl, optionally substituted C₂-C₆ alkenyl, optionally substituted C₂-C₆ alkynyl, C₁-C₆ haloalkyl, C₂-C₆ haloalkenyl, C₂-C₆ haloalkynyl, optionally substituted carbocyclyl, optionally substituted —CO(C₁-C₆ alkyl), —CO(optionally substituted heterocyclyl), optionally substituted heterocyclyl, optionally substituted aryl, or optionally substituted heteroaryl; or R⁷ and R^{8a} taken together form an optionally substituted heterocyclyl;
R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted —COO(C₁-C₆ alkyl), optionally substituted carbocyclyl, optionally substituted heterocyclyl, optionally substituted aryl, or optionally substituted heteroaryl; or (R¹¹ and R¹²) or (R¹⁴ and R¹⁵) taken together form an optionally substituted heterocyclyl;
each m is independently 0, 1 or 2;
n1 and n2 are each independently 0, 1, 2, 3, or 4;
n3 is 0, 1, 2, 3, 4 or 5; and
each t is independently 0, 1 or 2.

### Embodiment 108

The compound of embodiment 107, wherein C is 5- to 7-membered heteroaryl comprising 1, 2, or 3 heteroatoms selected from O, S, or N as a ring member.

### Embodiment 109

The compound of embodiment 107 or 108 wherein C is pyrazole, imidazole, oxazole, oxadiazole, oxazolone, isoxazole, thiazole, pyridyl, or pyrimidyl.

### Embodiment 110

The compound of any one of embodiment 107-109, wherein C, which is optionally substituted with R³, is selected from

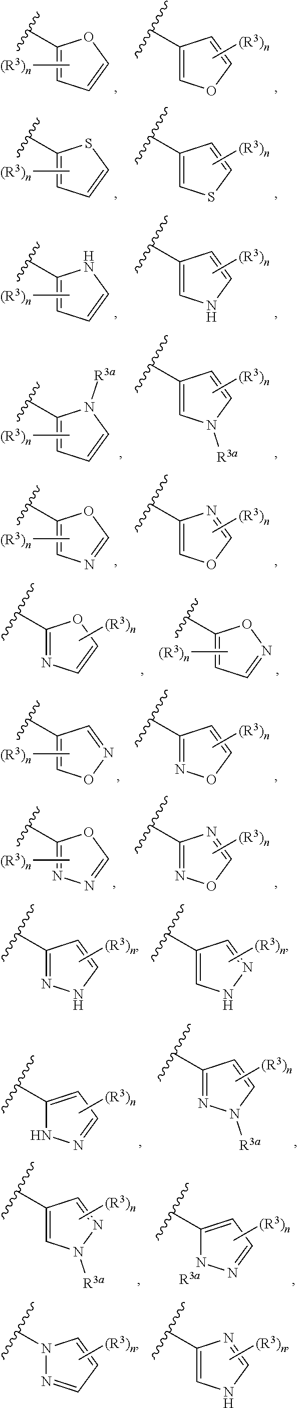

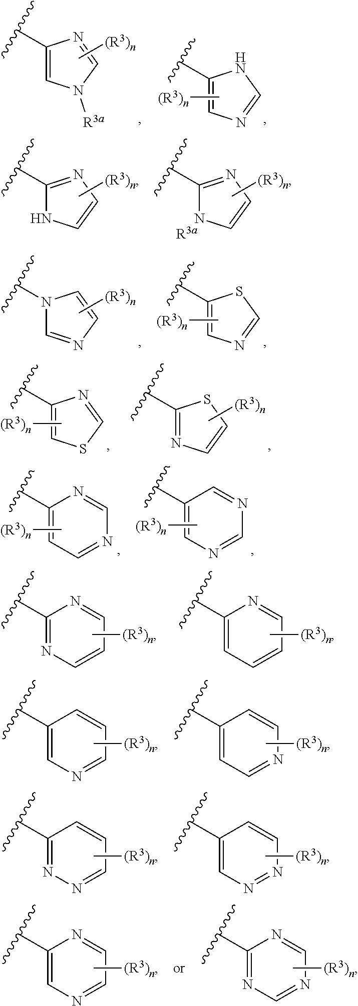

wherein R^{3a} is C₁-C₃ alkyl.

### Embodiment 111

The compound of any one of embodiments 107-110, wherein:
Z is a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—; and
L is halogen, —NH₂, or —CF₃.

### Embodiment 112

The compound of any one of embodiments 107-111, wherein X is a bond, —CH₂—, —C(CH₃)H—, —C(CH₃)₂—, or —CH₂CH₂—.

### Embodiment 113

The compound of any one of embodiments 107-112, wherein R¹ and R² are each independently halogen, —CN, —CF₃, —OH, methyl, methoxy, or —CONH₂.

### Embodiment 114

The compound of any one of embodiments 107-113, wherein R³ is selected from hydrogen, F, Cl, Br, I, oxo, ═S, ═NR¹⁶, —CN, —CF₃, —OH, C₁-C₃ alkyl, C₂-C₃ alkenyl, C₂-C₃ alkynyl, —S(C₁-C₃ alkyl), —SO(C₁-C₃ alkyl), —SO₂(C₁-C₃ alkyl), —NHSO₂CH₃, —N(CH₃)SO₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl).

### Embodiment 115

The compound of any one of embodiments 107-114, wherein at least one of R³ is —SO₂CH₃, —NHSO₂CH₃, —CH₂NHSO₂CH₃, —SO₂NH₂, —CONH₂, or —NHCOCH₃.

### Embodiment 116

The compound of embodiment 107, wherein the PTC has the structure of formula (II):

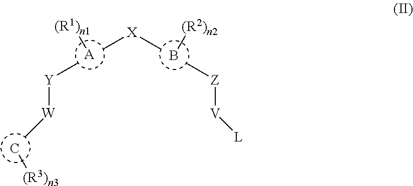

or a pharmaceutically acceptable salt, tautomer, stereoisomer or prodrug thereof, wherein:
A and B are each independently selected from phenyl, pyridyl, pyrimidyl, or thiophene;
C is a 5- to 10-membered heteroaryl or aryl;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂—, —CH₂CH₂—, —CH(CH₃)CH₂—, —CH₂CH(CH₃)—, or —CH₂CH₂CH₂—;
L is hydrogen, halogen, optionally substituted alkyl sulfonate, optionally substituted aryl sulfonate, —OH, —NH₂, or —CF₃;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —COOH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from hydrogen, halogen, oxo, ═S, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —NR¹⁴CONR¹⁴R¹⁵, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, —NH₂, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H, C₁-C₆ alkyl, —CO(C₁-C₆ alkyl);
R¹³, R¹⁴ and R¹⁵ are each independently hydrogen, C₁-C₃ alkyl, C₂-C₃ alkenyl, C₂-C₃ alkynyl, or —COO(C₁-C₆ alkyl); or R¹⁴ and R¹⁵ taken together form a 3- to 6-membered heterocyclyl;
R¹⁶ is hydrogen, C₁-C₃ alkyl, C₁-C₃ haloalkyl, C₂-C₃ alkenyl, or C₂-C₃ alkynyl;
n1 and n2 are each independently 0, 1, or 2;
n3 is 0, 1, 2, 3, 4 or 5; and
t is 0, 1 or 2.

### Embodiment 117

The compound of embodiment 116 wherein C is 5- to 7-membered heteroaryl comprising 1, 2, or 3 heteroatoms selected from O, S, or N as a ring member.

### Embodiment 118

The compound of embodiment 116 or 117, wherein C is pyrazole, imidazole, oxazole, oxadiazole, oxazolone, isoxazole, thiazole, pyridyl, or pyrimidyl.

### Embodiment 119

The compound of embodiment 116, wherein C, which is optionally substituted with R³, is selected from

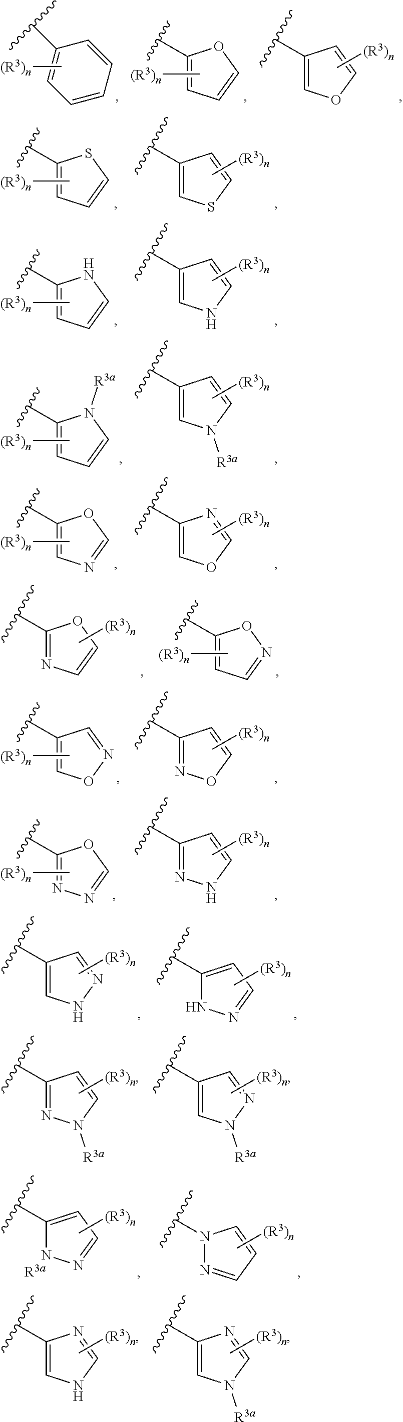

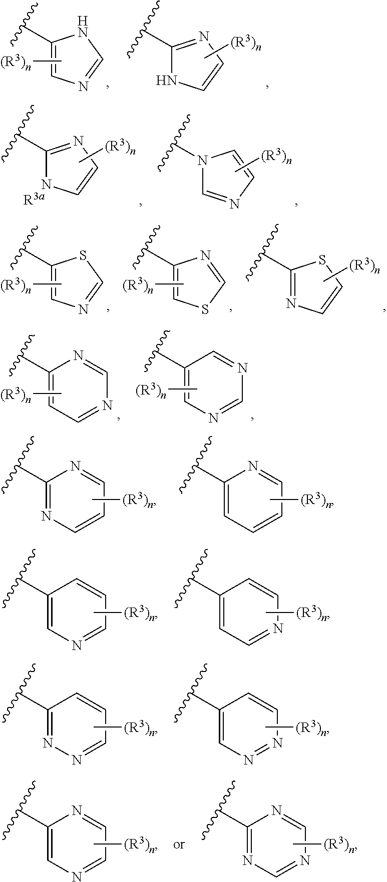

wherein R³a is C₁-C₃ alkyl.

### Embodiment 120

The compound of any one of embodiments 116-119, wherein A has a meta or para connectivity with X and Y.

### Embodiment 121

The compound of any one of embodiments 116-120, wherein B has a meta or para connectivity with X and Z.

### Embodiment 122

The compound of any one of embodiments 116-121, wherein A and B are each phenyl.

### Embodiment 123

The compound of any one of embodiments 116-122, wherein —Z—V-L is —Z—CH₂CH₂C₁, —Z—CH₂CH₂CH₂C₁, —Z—CH₂CH₂NH₂, or —Z—CH₂CH₂CH₂NH₂, wherein Z is a bond, —O—, —NH—, or —N(COCH₃)—.

### Embodiment 124

The compound of any one of embodiments 116-123, wherein —Y—W— is a bond, —OCH₂—, —OCH₂CH₂—, —OCH(CH₃)—, —NH—, —NHCH₂—, —NHC(═O)—, or —C(═O)NH—.

### Embodiment 125

The compound of any one of embodiments 116-124, wherein X is a bond, —CH₂—, —C(CH₃)H—, —C(CH₃)₂—, or —CH₂CH₂—.

### Embodiment 126

The compound of embodiment 107, wherein the PTC has the structure of formula (III)

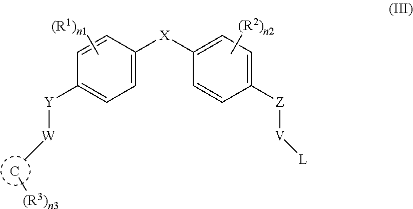

or a pharmaceutically acceptable salt, tautomer, stereoisomer or prodrug thereof, wherein.
C is a phenyl or a 5- to 7-membered monocyclic heteroaryl comprising 1, 2, or 3 heteroatoms selected from O, S, or N as a ring member;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂—, —CH₂CH₂—, or —CH₂CH₂CH₂—;
L is halogen, optionally substituted alkyl sulfonate, optionally substituted aryl sulfonate, —NH₂, or —CF₃;
R¹ and R² are each independently hydrogen, halogen, —OH, —NH₂, —CN, —CF₃, methyl, or —CONH₂;
R³ is selected from hydrogen, F, Cl, Br, I, oxo, —CN, —CF₃, —OH, C₁-C₃ alkyl, C₂-C₃ alkenyl, C₂-C₃ alkynyl, —S(C₁-C₃ alkyl), —SO₂(C₁-C₃ alkyl), —NHSO₂(C₁-C₃ alkyl), —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), or —N(CH₃)CO(C₁-C₃ alkyl);
R⁵ and R⁶ are each independently hydrogen, halogen, —OH, —NH₂, or C₁-C₃ alkyl;
R⁷ is H or C₁-C₆ alkyl;
n1 and n2 are each independently 0, 1, or 2;
n3 is 0, 1, 2, 3, 4 or 5; and
t is 0, 1 or 2.

### Embodiment 127

The compound of embodiment 126, wherein C is 5- to 7-membered monocyclic heteroaryl comprising 1, 2, or 3 heteroatoms selected from O, S, or N as a ring member.

### Embodiment 128

The compound of embodiment 126 or 127, wherein —V-L is —CH₂CH₂C₁, —CH₂CH₂CH₂C₁, —CH₂CH₂NH₂, or —CH₂CH₂CH₂NH₂.

### Embodiment 129

The compound of any one of embodiments 126-128, wherein —Y—W— is a bond, —OCH₂—, —OCH₂CH₂—, —OCH(CH₃)—, —NH—, —NHCH₂—, —NHC(═O)—, or —C(═O)NH—.

### Embodiment 130

The compound of any one of embodiments 126-129, wherein X is a bond, —CH₂—, —C(CH₃)H—, —C(CH₃)₂—, or —CH₂CH₂—.

### Embodiment 131

The compound of embodiment 107, wherein the PTC has the structure of formula (IV)

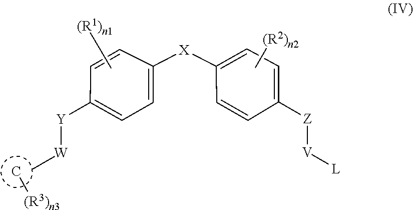

or a pharmaceutically acceptable salt, tautomer, stereoisomer or prodrug thereof, wherein:
C is

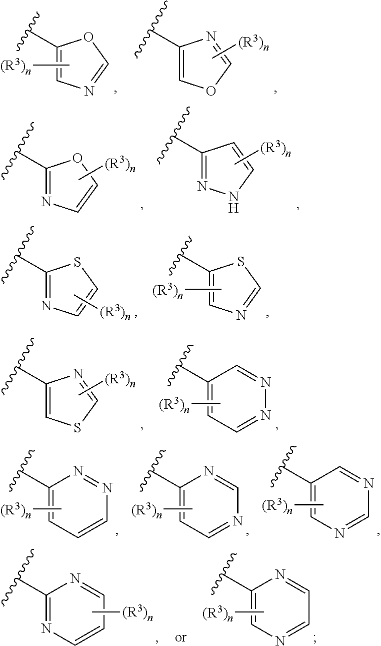

X is —(CR⁵R⁶)ₜ— or —NR⁷—;
Y is a bond, —CH₂—, —O—, or —NH—;
Z is a bond, —CH₂—, —O—, or —NH—;
W is a bond, —CH₂—, or —C(CH₃)H—;
V is —CH₂—, —CH₂CH₂—, —CH₂CH₂CH₂—, or —CH₂CHClCH₂—;
L is hydrogen, —OH, halogen, optionally substituted alkyl sulfonate, or optionally substituted aryl sulfonate;
R¹ and R² are each independently hydrogen, halogen, —CN, —CF₃, methyl, —OH, —NH₂, —COOH, or —CONH₂;
R³ is selected from hydrogen, F, Cl, Br, I, oxo, —CN, —CF₃, —OH, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —NHSO₂(C₁-C₃ alkyl), —NHSO₂CF₃, —N(CH₃)SO₂(C₁-C₃ alkyl), —CH₂NHSO₂(C₁-C₃ alkyl), —CH₂N(CH₃)SO₂(C₁-C₃ alkyl), —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, —OH, —NH₂, or C₁-C₃ alkyl;
R⁷ is H or C₁-C₆ alkyl;
n1 and n2 are each independently 0, 1, or 2;
n3 is 0, 1, or 2; and
t is 1 or 2.

### Embodiment 132

The compound of embodiment 131, wherein R³ is selected from hydrogen, F, Cl, Br, I, —CN, —CF₃, —OH, methyl, methoxy, —S(C₁-C₃ alkyl), —SO₂(C₁-C₃ alkyl), —NH₂, —NHSO₂CH₃, —NHSO₂CF₃, —N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —NHCO(C₁-C₃ alkyl).

### Embodiment 133

The compound of embodiment 106, wherein the compound has the structure of formula (V):

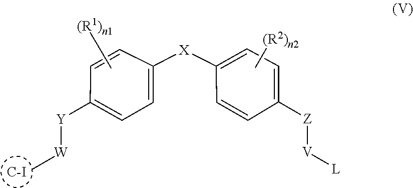

or a pharmaceutically acceptable salt, tautomer, stereoisomer or prodrug thereof, wherein:
C-I is

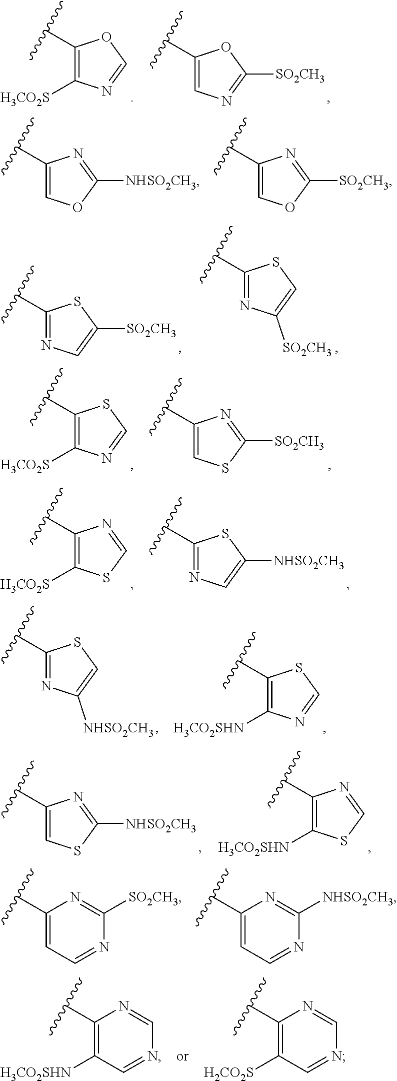

X is —(CR⁵R⁶)ₜ—;
Y is —O—;
Z is —O—;
W is —CH₂— or —C(CH₃)H—;
V is —CH₂—, —CH₂CH₂— or —CH₂CH₂CH₂—;
L is halogen, optionally substituted alkyl sulfonate, or optionally substituted aryl sulfonate;
R¹ and R² are each independently halogen, —OH, —NH₂, or —CN;
R⁵ and R⁶ are each independently hydrogen, methyl, —OH, —NH₂;
R⁷ is H or C₁-C₆ alkyl;
n1 and n2 are each independently 0, 1, or 2; and
   t is 1.

### Embodiment 134

The compound of any one of embodiments 75-105, wherein the PTC is selected from Table A.

### Embodiment 135

The compound of any one of embodiments 75-105, wherein the PTC is selected from Table B.

### Embodiment 136

The compound of any one of embodiments 75-105, wherein the PTC has the structure of formula (i):

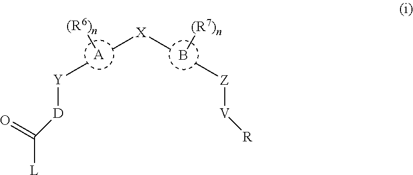

or a pharmaceutically acceptable salt, tautomer, stereoisomer or prodrug thereof, wherein:
A and B are each independently aryl or heteroaryl;
X is a bond, —(CR⁸R⁹)ₜ—, —O—, —C(═O)—, —S(O)ₙ—, —NR¹⁰—, —CONR¹⁰—, —NR¹⁰CO—, —SO₂NR¹⁰—, or —NR¹⁰SO₂—;
Y and Z are each independently a bond, —(CR⁸R⁹)ₜ—, —O—, —S(O)ₙ—, —NR¹⁰—, —CONR¹⁰—, —NR¹⁰CO—, —SO₂NR¹⁰—, or —NR¹⁰SO₂—;
V is a bond, optionally substituted —(CR¹¹R¹²)ₘ—, —C(═O)—, —N(R¹⁰)CO—, —CONR¹⁰—, or —NSO₂R¹⁰—;
R is —(CR^{4a}R^{4b})—(CR^{5a}R^{5b})—W or W;
W is hydrogen, halogen, optionally substituted alkylsulfonate, optionally substituted arylsufonate, —CF₃, —CF₂R¹⁰, —CN, —OR¹³, —NR¹³R¹⁴, optionally substituted —CONR¹³R¹⁴, optionally substituted carbocyclyl, optionally substituted heterocyclyl, optionally substituted aryl, or optionally substituted heteroaryl;
D is —(CR^{1a}R^{1b})_{q}—, —O—, or —NR¹⁰—;
L is —(CR^{2a}R^{2b})—R³ or -E-R³;
E is —(CR^{2a}R^{2b})_{g}—, —O—, —NR¹⁰—, or —NR¹⁰—(CR^{2a}R^{2b})_{g}—;
R^{1a}, R^{1b}, R^{2a}, and R^{2b} are each independently hydrogen, halogen, hydroxy, optionally substituted C₁₋₆ alkyl, optionally substituted C₂-C₆ alkenyl, optionally substituted C₂-C₆ alkynyl, optionally substituted C₁₋₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
or alternatively, R^{1a} and R^{1b} taken together form an optionally substituted carbocyclyl, optionally substituted heterocyclyl, optionally substituted aryl, or optionally substituted heteroaryl;
or alternatively, R^{2a} and R^{2b} taken together form a CO, optionally substituted carbocyclyl, optionally substituted heterocyclyl, optionally substituted aryl, or optionally substituted heteroaryl;
or alternatively, R^{1a}, R^{1b}, R^{2a} and R^{2b} taken together form an optionally substituted carbocyclyl, optionally substituted heterocyclyl, optionally substituted aryl, or optionally substituted heteroaryl;
R^{4a}, R^{4b}, R^{5a}, and R^{5b} are each independently hydrogen, halogen, hydroxy, optionally substituted C₁₋₆ alkyl, optionally substituted C₂-C₆ alkenyl, optionally substituted C₂-C₆ alkynyl, optionally substituted C₁₋₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
or alternatively, R^{4a} and R^{4b} taken together form a CO, optionally substituted carbocyclyl, optionally substituted heterocyclyl, optionally substituted aryl, or optionally substituted heteroaryl;
or alternatively, R^{4a}, R^{4b}, R^{5a} and R^{5b} taken together form an optionally substituted carbocyclyl, optionally substituted heterocyclyl, optionally substituted aryl, or optionally substituted heteroaryl;
R³ is absent, hydrogen, —CN, —CF₃, —OH, optionally substituted C₁-C₆ alkyl, optionally substituted C₂-C₆ alkenyl, optionally substituted C₂-C₆ alkynyl, optionally substituted —OR¹⁵, optionally substituted C₁-C₆ alkoxy, —NH₂, —NR¹⁶R¹⁷, —NR¹⁶COR¹⁸, —NR¹⁶S(O)ₚR¹⁸, —CONR¹⁴R¹⁵, —SONR¹⁴R¹⁵, —SO₂NR¹⁴R¹⁵, optionally substituted —S(O)ₚR¹⁸, —N₃, optionally substituted carbocyclyl, optionally substituted heterocyclyl, optionally substituted aryl, or optionally substituted heteroaryl;
or alternatively, R^{2a}, R^{2b} and R³ taken together form an optionally substituted carbocyclyl, optionally substituted heterocyclyl, optionally substituted aryl, or optionally substituted heteroaryl;
or alternatively, R^{2a} and R¹⁰ taken together form an optionally substituted heterocyclyl;
R⁶ and R⁷ are each independently H, methyl, methoxy, —CN, F, Cl, Br, I, ¹²³I, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶, optionally substituted carbocyclyl, optionally substituted heterocyclyl, optionally substituted aryl, or optionally substituted heteroaryl;
R⁸, R⁹, R¹¹ and R¹² are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl;
or alternatively, R⁸ and R⁹ taken together form an optionally substituted carbocyclyl or optionally substituted heterocyclyl;
or alternatively, R¹¹ and R¹², on a same carbon atom or a different carbon atom, taken together form an optionally substituted carbocyclyl, optionally substituted heterocyclyl, optionally substituted aryl, or optionally substituted heteroaryl;
R¹⁰ is hydrogen, halogen, optionally substituted C₁-C₆ alkyl, optionally substituted C₁-C₆ alkoxy, —CO(C₁-C₆ alkyl), optionally substituted C₁-C₆ alkylamino, optionally substituted carbocyclyl, optionally substituted heterocyclyl, optionally substituted aryl, or optionally substituted heteroaryl;
R¹³, R¹⁴, R¹⁵, R¹⁶, R¹⁷ and R¹⁸ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
or alternatively, R¹⁴ and R¹⁵ are taken together to form an optionally substituted heterocyclyl, or optionally substituted heteroaryl;
or alternatively, R¹⁶ and R¹⁷ are taken together to form an optionally substituted heterocyclyl, or optionally substituted heteroaryl;
m is 0, 1, 2, 3, or 4;
each n is independently 0, 1 or 2;
each p is independently 0, 1 or 2;
q is 0, 1 or 2;
each g is independently 0, 1, 2, 3, or 4; and
each t is independently 1 or 2.

### Embodiment 137

The compound of embodiment 136, wherein R is W.

### Embodiment 138

The compound of embodiment 136 or 137, wherein W is hydrogen, halogen, —CF₃, or —NR¹³R¹⁴.

### Embodiment 139

The compound of any one of embodiments 136-138, wherein L is -E-R³.

### Embodiment 140

The compound of any one of embodiments 136-139, wherein R³ is selected from hydrogen, —C₁-C₃ alkyl, —NR¹⁶SO(C₁-C₃ alkyl), —NR¹⁶SO₂(C₁-C₃ alkyl), —SONR¹⁴R¹⁵, —SO₂NR¹⁴R¹⁵, —SOR¹⁸, or —SO₂R¹⁸.

### Embodiment 141

The compound of any one of embodiments 136-140, wherein R³ is selected from —NHSO₂(C₁-C₃ alkyl), —NCH₃SO₂(C₁-C₃ alkyl), or —SO₂(C₁-C₃ alkyl).

### Embodiment 142

The compound of embodiment 136, wherein the PTC has the structure of formula (ii):

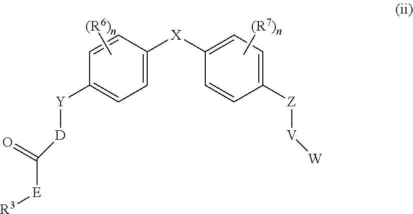

or a pharmaceutically acceptable salt, tautomer, stereoisomer or prodrug thereof, wherein:
X is a bond, —NR¹⁰—, or —(CR^{8a}R^{9a})ₜ—;
Y and Z are each independently a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—;
W is halogen, optionally substituted alkylsulfonate, optionally substituted arylsufonate, —NH₂, or —CF₃.
D is —NR¹⁰— and E is —(CR^{2a}R^{2b})_{g}—, —NR¹⁰—, or —NR¹⁰—(CR^{2a}R^{2b})_{g}—,
or alternatively, E is —NR¹⁰— or —NR¹⁰—(CR^{2a}R^{2b})_{g}—, and D is —(CR^{1a}R^{1b})_{q}— or —NR¹⁰—;
R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or (R^{1a} and R^{1b}) or (R^{2a} and R^{2b}) taken together form an oxo (═O), an optionally substituted carbocyclyl, or an optionally substituted heterocyclyl;
R³ is selected from hydrogen, —C₁-C₆ alkyl, —OR¹⁵, —SR¹⁸, —C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, —SOR¹⁸, or —SO₂R¹⁸;
R⁶ and R⁷ are each independently H, halogen, —CN, —CF₃, —OH, —COOH, —NH₂, —CONH₂, or C₁-C₃ alkyl;
R^{8a} and R^{9a} are each independently hydrogen, halogen, —OH, —NH₂, or C₁-C₃ alkyl; or R^{8a} and R^{9a} taken together form an 3- to 6-membered carbocyclyl or heterocyclyl;
R¹⁰ is each independently hydrogen, C₁-C₃ alkyl, C₂-C₃ alkenyl, C₂-C₃ alkynyl, or —CO(C₁-C₃ alkyl);
R¹³, R¹⁴, R¹⁵, R¹⁶, R¹⁷ and R¹⁸ are each independently hydrogen, C₁-C₃ alkyl, C₂-C₃ alkenyl, or C₂-C₃ alkynyl; or R¹⁴ and R¹⁵ taken together form an optionally substituted 5- or 6-membered heterocyclyl;
each n is independently 0, 1 or 2;
q is 0, 1 or 2;
each g is independently 0, 1, 2, 3, or 4; and
each t is independently 1 or 2.

### Embodiment 143

The compound of embodiment 136, wherein the PTC has the structure of formula (iii):

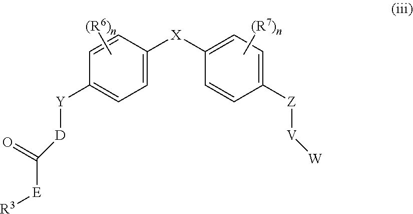

or a pharmaceutically acceptable salt, tautomer, stereoisomer or prodrug thereof, wherein:
X is a bond, —NR¹⁰—, or —(CR^{8a}R^{9a})ₜ—;
Y and Z are each independently a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—;
W is halogen, optionally substituted alkylsulfonate, optionally substituted arylsufonate, —NH₂ or —CF₃;
D is —O— or —NR¹⁰— and E is —(CR^{2a}R^{2b})_{gg};
or alternatively, E is —O—, —NR¹⁰— or —NR¹⁰—(CR^{2a}R^{2b})_{g}—, and D is —(CR^{1a}R^{1b})_{q}—;
R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R⁵; or (R^{1a} and R^{1b}) or (R^{2a} and R^{2b}) taken together form an oxo (═O), an optionally substituted carbocyclyl, or an optionally substituted heterocyclyl;
R³ is selected from hydrogen, —C₁-C₆ alkyl, —OR¹⁵, —SR¹⁸, —C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, —SOR¹⁸, or —SO₂R¹⁸;
R⁶ and R⁷ are each independently H, halogen, —CN, —CF₃, —OH, —COOH, —NH₂, —CONH₂, or C₁-C₃ alkyl;
R^{8a} and R^{9a} are each independently hydrogen, halogen, —OH, —NH₂, or C₁-C₃ alkyl; or R^{8a} and R^{9a} taken together form an 3- to 6-membered carbocyclyl or heterocyclyl;
R¹⁰ is each independently hydrogen, C₁-C₃ alkyl, C₂-C₃ alkenyl, C₂-C₃ alkynyl, or —CO(C₁-C₃ alkyl);
R¹³, R¹⁴, R¹⁵, R¹⁶, R¹⁷ and R¹⁸ are each independently hydrogen, C₁-C₃ alkyl, C₂-C₃ alkenyl, or C₂-C₃ alkynyl; or R¹⁴ and R¹⁵ taken together form an optionally substituted 5- or 6-membered heterocyclyl;
m is 0, 1, 2, 3, or 4;
each n is independently 0, 1 or 2;
q is 1 or 2;
g is 0, 1, 2, 3, or 4;
gg is 1, 2, 3, or 4; and
t is 1 or 2.

### Embodiment 144

The compound of embodiment 142 or 143, wherein W is Cl, Br, I, or F.

### Embodiment 145

The compound of any one of embodiments 142-144, wherein D is —CH₂—, —CH(CH₃)—, —C(CH₃)₂—, or —CH₂CH₂—.

### Embodiment 146

The compound of embodiment 142, wherein q is 0.

### Embodiment 147

The compound of any one of embodiments 142-146, wherein E is —CH₂—, —CH(CH₃)—, —C(CH₃)₂—, —CH₂CH₂—, or —CH₂CH₂CH₂—.

### Embodiment 148

The compound of any one of embodiments 142-147, wherein g is 0.

### Embodiment 149

The compound of any one of embodiments 142-148, wherein R³ is selected from —NHSO₂(C₁-C₃ alkyl), —NCH₃SO₂(C₁-C₃ alkyl), or —SO₂(C₁-C₃ alkyl).

### Embodiment 150

The compound of any one of embodiments 142-149, wherein R⁶ and R⁷ are each independently H, halogen, —CN, or methyl.

### Embodiment 151

The compound of any one of embodiments 142-150, wherein X is a bond, —CH₂—, —C(CH₃)₂—, —CH₂CH₂—, —NH—, —N(CH₃)—, —N(iPr)-, or —N(COCH₃)—.

### Embodiment 152

The compound of any one of embodiments 142-151, Z is —CH₂—, —O—, —NH—, —NCH₃—, or —N(COCH₃)—.

### Embodiment 153

The compound of any one of embodiments 142-152, Y is —CH₂—, —O—, —NH—, or —NCH₃—.

### Embodiment 154

The compound of any one of embodiments 142-153, wherein at least one of Z and Y is —O—.

### Embodiment 155

The compound of embodiment 132, wherein the PTC has the structure of formula (iv):

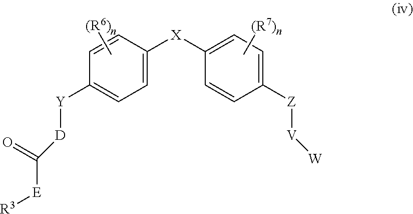

or a pharmaceutically acceptable salt, tautomer, stereoisomer or prodrug thereof, wherein:
X is a bond, —NR¹⁰—, or —(CR^{8a}R^{9a})ₜ—;
Y and Z are each independently a bond, —CH₂—, —C(CH₃)H—, —O—, —S—, —NH—, —NCH₃—, or —N(COCH₃)—;
V is —CH₂—, —CH₂CH₂—, —CH₂CH₂CH₂—, —CH₂CH(CH₃)CH₂—, —CH₂CH(OH)CH₂—, or —CH₂C(OH)(CH₃)CH₂—;
W is halogen, optionally substituted alkylsulfonate, optionally substituted arylsufonate, —CF₂R¹⁰, —NR¹³R¹⁴, optionally substituted carbocyclyl, optionally substituted heterocyclyl, optionally substituted aryl, or optionally substituted heteroaryl;
D is —(CR^{1a}R^{1b})_{q}—;
E is —(CR^{2a}R^{2b})_{g}—;
R^{1a}, R^{1b}, R^{2a}, and R^{2b} are each independently hydrogen, halogen, —OH, C₁-C₃ alkyl, C₂-C₃ alkenyl, C₂-C₃ alkynyl, C₁-C₃ alkoxy, —OCO(C₁-C₃ alkyl), —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₆ alkyl)-CONR¹⁴R¹⁵; or (R^{1a} and R^{1b}) or (R^{2a} and R^{2b}) taken together form an oxo (═O), an optionally substituted carbocyclyl, or an optionally substituted heterocyclyl;
R³ is selected from hydrogen, —C₁-C₆ alkyl, —OR¹⁵, —SR¹⁸, —C₁-C₆ alkoxy, —NR¹⁶R¹⁷, —NR¹⁶SR¹⁸, —NR¹⁶SOR¹⁸, —NR¹⁶SO₂R¹⁸, —NR¹⁶COR¹⁸, —CONR¹⁴R¹⁵, —SONR¹⁴R¹⁵, —SO₂NR¹⁴R¹⁵, —SOR¹⁸, or —SO₂R¹⁸;
R⁶ and R⁷ are each independently H, halogen, —CN, —CF₃, —OH, —COOH, —NH₂, —CONH₂, or C₁-C₃ alkyl;
R^{8a} and R^{9a} are each independently hydrogen, halogen, —OH, —NH₂, or C₁-C₃ alkyl; or
R^{8a} and R^{9a} taken together form an optionally substituted carbocyclyl or optionally substituted heterocyclyl;
R¹⁰ is each independently hydrogen, C₁-C₃ alkyl, C₂-C₃ alkenyl, C₂-C₃ alkynyl, or —CO(C₁-C₃ alkyl);
R¹³, R¹⁴, R¹⁵, R¹⁶, R¹⁷ and R¹⁸ are each independently hydrogen, C₁-C₃ alkyl, C₂-C₃ alkenyl, or C₂-C₃ alkynyl; or R¹⁴ and R¹⁵ taken together form an optionally substituted 5- or 6-membered heterocyclyl;
m is 0, 1, 2, 3, or 4;
each n is independently 0, 1 or 2;
q is 0, 1 or 2;
g is 0, 1, 2, 3, or 4; and
t is 1 or 2.

### Embodiment 156

The compound of any one of embodiments 75-106, wherein the PTC is selected from Table C.

### Embodiment 157

The compound of any one of embodiments 75-106, wherein the PTC has the structure of formula (a):

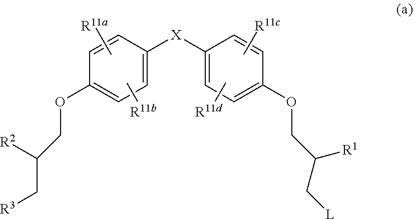

wherein:
X is —S(O)ₙ— or —C(R⁸R⁹)—; L is halogen, optionally substituted alkyl sulfonate, or optionally substituted aryl sulfonate;
R¹ is H, hydroxyl or —OC(═O)R¹³;
R² is hydroxyl or —OC(═O)R¹³;
R³ is halo, —OH, —OR⁴; —OC(═O)R¹³, —NH₂, —NHC(═O)R¹³, —N(C(═O)R¹³)₂, —NHS(O)ₙR⁵, —N(C(═O)R¹³)(S(O)ₙR⁵), —N(C₁-C₆ alkyl)(S(O)ₙR⁵), —S(O)ₙR⁵, —N₃, aryl, carbocyclyl, heteroaryl or heterocyclyl which are optionally substituted with one or more R⁶.
R⁴ is C₁-C₆ alkyl, C₂-C₆ alkenyl, C₂-C₆ alkynyl, aryl, carbocyclyl, heteroaryl or heterocyclyl which are optionally substituted with one or more R⁶.
R⁵ is each independently C₁-C₆ alkyl or aryl which are optionally substituted with one or more R⁶.
R⁶ is each independently selected from the group consisting of H, F, Cl, Br, I, ¹²³I, hydroxyl, oxo, C₁-C₆ alkyl, C₂-C₆ alkenyl, C₂-C₆ alkynyl, C₁-C₆ alkoxy, C₆-C₁₂ aryl, wherein each R⁶ is optionally substituted with one or more of halogen, ¹²³I, ¹⁸F, hydroxyl, —OS(O)₂-aryl, C₁-C₆ alkyl, C₂-C₆ alkenyl, or C₂-C₆ alkynyl;
R⁸ and R⁹ are each independently H, —OH, —NH₂, or C₁-C₆ alkyl;
R^{11a}, R^{11b}, R^{11c} and R^{11d} are each independently H, methyl, F, Cl, Br, I ¹²³I, —OH, —NH₂, —CN, —CF₃, methyl, —COOH, or —CONH₂;
R¹³ is C₁-C₆ alkyl; and
n is 0, 1, or 2;
   wherein at least one of R^{11a}, R^{11b}, R^{11c} and R^{11d} is methyl F, Cl, Br, I, or ¹²³I.

### Embodiment 158

The compound of embodiment 157, wherein at least two of R^{11a}, R^{11b}, R^{11c} and R^{11d} are methyl, F, Cl, Br, I, or ¹²³I.

### Embodiment 159

The compound of embodiment 157, wherein R^{11c} and R^{11d} are each independently methyl, Cl, or Br.

### Embodiment 160

The compound of embodiment 157, wherein R^{11c} and R^{11d} are each Cl.

### Embodiment 161

The compound of any one of embodiments 157-160, wherein X is —C(R⁸R⁹)— and R⁸ and R⁹ are each independently C₁-C₃ alkyl.

### Embodiment 162

The compound of embodiment 161, wherein R⁸ an R⁹ are each methyl.

### Embodiment 163

The compound of any one of embodiment 157-162, wherein R¹ and R² are both hydroxyl.

### Embodiment 164

The compound of any one of embodiment 157-163, wherein R³ is an optionally substituted 5 or 6 membered heteroaryl or an optionally substituted 3 to 7 membered heterocylyl, wherein said heteroaryl or said heterocyclyl respectively comprise at least one N atom.

### Embodiment 165

The compound of any one of embodiment 157-163, wherein R³ is selected from a group consisting of pyrrole, furan, thiophene, pyrazole, pyridine, pyridazine, pyrimidine, imidazole, thiazole, isoxazole, oxadiazole, thiadiazole, oxazole, triazole, isothiazole, oxazine, triazine, azepine, pyrrolidine, pyrroline, imidazoline, imidazolidine, pyrazoline, pyrazolidine, piperidine, dioxane, morpholine, dithiane, thiomorpholine, piperazine, and tetrazine.

### Embodiment 166

The compound of any one of embodiment 157-163, wherein R³ is —NH₂, —NHC(═O)R¹³, —N(C(═O)R¹³)₂, —NHS(O)ₙR, —N(C(═O)R¹³)(S(O)ₙR⁵), —N(C₁-C₆ alkyl)(S(O)ₙR) or —S(O)ₙR⁵.

### Embodiment 167

The compound of any one of embodiment 157-163, wherein R³ is —NH₂, —NHC(═O)(C1-C4 alkyl), —N[(C(═O)(C1-C4 alkyl)]₂, —NHS(O)ₙ(C1-C3 alkyl), —N[C(═O)(C1-C4 alkyl)][(S(O)ₙ(C1-C3 alkyl)], —N[C₁-C₆ alkyl][S(O)ₙ(C1-C3 alkyl)], or —S(O)ₙ(C1-C3 alkyl).

### Embodiment 168

The compound of any one of embodiments 75-106, wherein the PTC is selected from Table D.

### Embodiment 169

A pharmaceutical composition comprising a compound of any one of embodiments 75-168 and a pharmaceutically acceptable carrier.

### Embodiment 170

The pharmaceutical composition of embodiment 169, further comprising one or more additional therapeutic agents.

### Embodiment 171

The pharmaceutical composition of embodiment 170, wherein the one or more additional therapeutic agents is for treating prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

### Embodiment 172

The pharmaceutical composition of embodiment 170, wherein the one or more additional therapeutic agents is a poly (ADP-ribose) polymerase (PARP) inhibitor including but not limited to olaparib, niraparib, rucaparib, talazoparib; an androgen receptor ligand binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide; a 5-alpha reductase inhibitor including but not limited to finasteride, dutasteride, turosteride, bexlosteride, izonsteride, FCE 28260, SKF105,111; a vascular endothelial growth factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an N-terminal domain inhibitor including but not limited to a sintokamide; an alpha-particle emitting radioactive therapeutic agent including but not limited to radium 233 or a salt thereof, niclosamide; or related compounds thereof, a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromitase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; CDK4/6 inhibitors including palbociclib, abemaciclib, ribociclib; HER2 receptor antagonist including but not limited to trastuzumab, neratinib; or a mammalian target of rapamycin (mTOR) inhibitor including but not limited to everolimus, temsirolimus.

### Embodiment 173

A method for modulating androgen receptor activity, comprising administering a compound of any one of embodiments 157-168, to a subject in need thereof.

### Embodiment 174

The method of any one of embodiment 173, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

### Embodiment 175

A method for treating cancer, comprising administering a compound of any one of embodiments 157-168, to a subject in need thereof.

### Embodiment 176

The method of embodiment 175, wherein the cancer is selected from prostate cancer, breast cancer, ovarian cancer, bladder cancer, pancreatic cancer, hepatocellular cancer, endometrial cancer, or salivary gland carcinoma.

### Embodiment 177

The method of embodiment 175, wherein the cancer is prostate cancer.

### Embodiment 178

The method of embodiment 177, wherein the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer.

### Embodiment 179

The method of embodiment 177, wherein the prostate cancer is metastatic castration-resistant prostate cancer.

### Embodiment 180

The method of embodiment 177, wherein the prostate cancer expresses full-length androgen receptor or truncated androgen receptor splice variant.

### Embodiment 181

A compound of formula (Y-IV), (Y-IVA), (Y-V), (Y-VA), (Y-VI), (Y-VIA), (Y-VII), (Y-VIII), (Y-IX), or (Y-X):

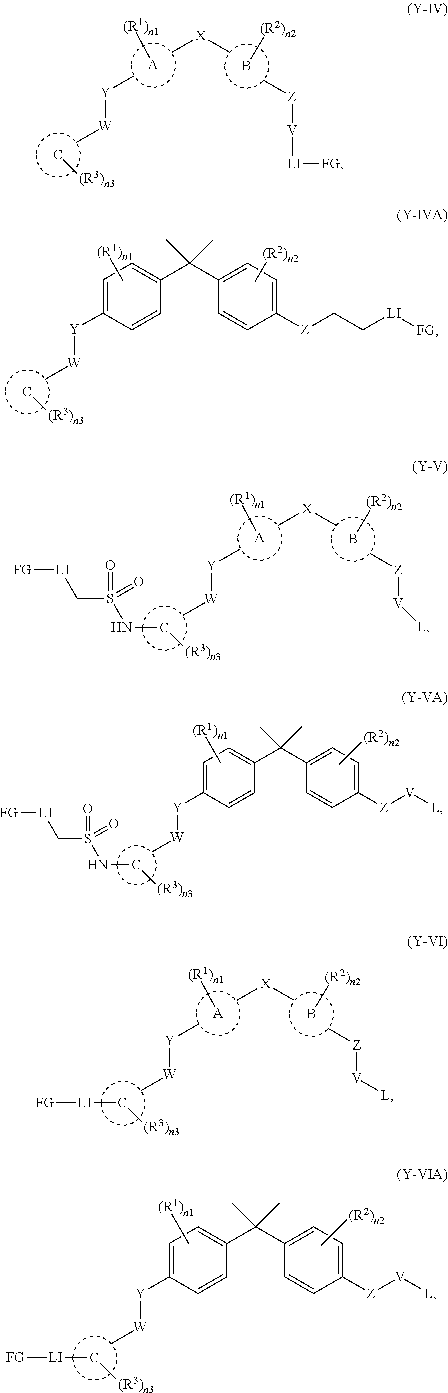

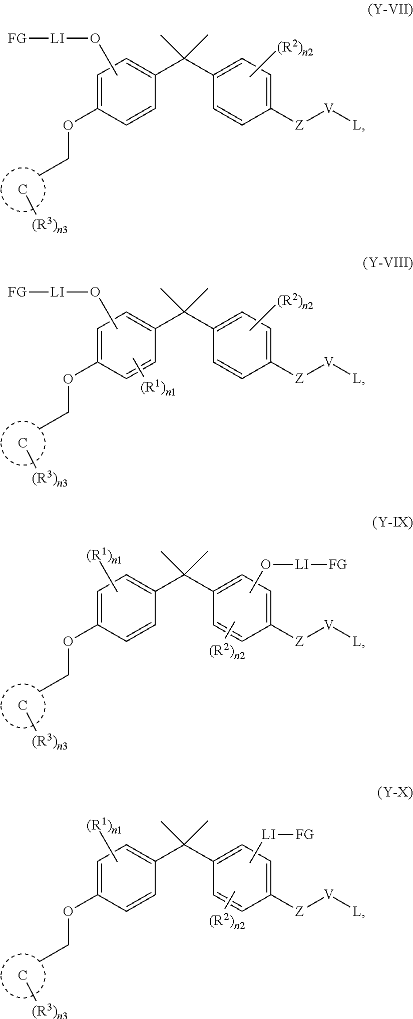

or a pharmaceutically acceptable salt thereof, wherein A, B, C, R¹, R², R³, Z, V, L, Y, W, LI, FG, n1, n2, and n3 are as defined in any one of embodiments 1-180.

### Embodiment 182

A compound selected from

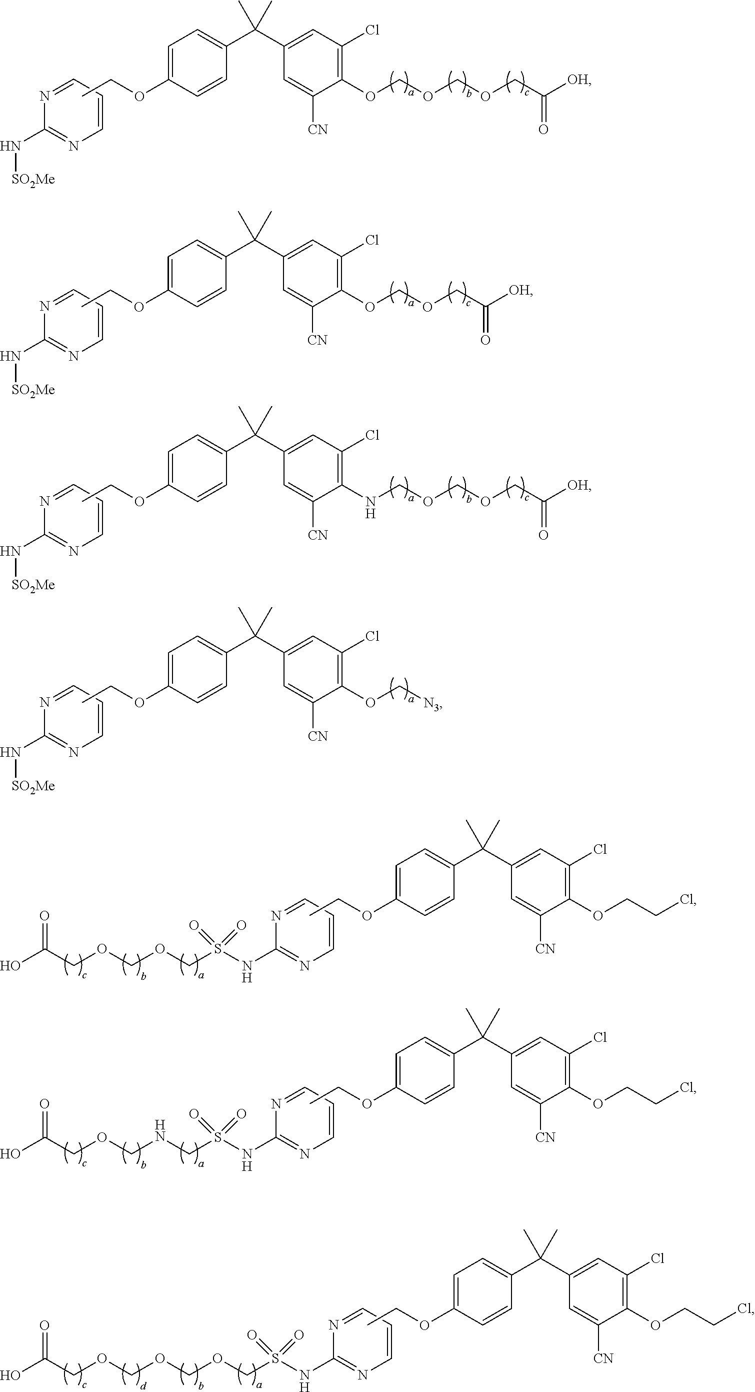

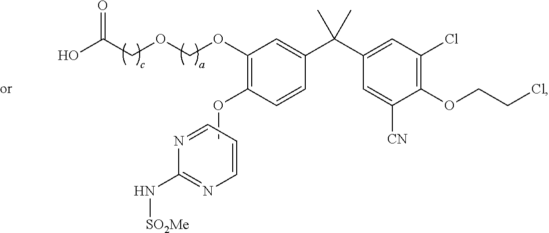

or a pharmaceutically acceptable salt thereof, wherein a, b, c, and d are each independently an integer between 1 to 10.

### Embodiment 183

The compound of embodiment 182, wherein a is 5, b is 3, and c is 1.

### Embodiment 184

The compound of embodiment 182, wherein a is 2, b is 5, and c is 1.

### Embodiment 185

The compound of embodiment 182, wherein, a is 2, b is 5, c is 1, and d is 3.

### Embodiment 186

The compound of embodiment 182, wherein a is 5 and c is 1.

### Embodiment 187

The compound of embodiment 182, wherein a is 5.

### Embodiment 188

The compound of embodiment 182, wherein a is 3.

What is claimed is:

1. A compound of formula (Q):
PLM-LI-PTC (Q);
or a pharmaceutically acceptable salt thereof, wherein:
PLM is a E3 ligase binding group,
LI is a linker, and
PTC is an androgen receptor modulator represented by formula (IIIA): 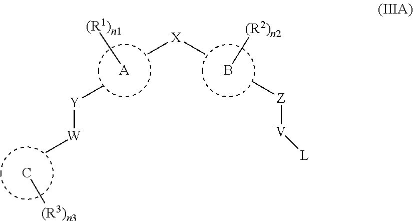
or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{8b} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

2. The compound of claim 1, wherein the linker LI corresponds to formula
-LX_{A}-(CH₂)ₘ₁—(CH₂—CH₂-LX_{B})ₘ₂—(CH₂)ₘ₃-LX_{C}-, wherein:
-LX_{A} is covalently bound to the PTC or PLM, and LX_{C}- is covalently bound to the PLM or PTC;
each m1 and m2 is independently 0, 1, 2, 3, 4, 5, 6, 7, 8, 9, 10, 11, or 12;
m3 is independently 1, 2, 3, 4, 5, 6, 7, 8, 9, 10, 11, or 12;
LX_{A} is absent (a bond), —CH₂C(O)NR²⁰—, or —NR²⁰C(O)CH₂—;
LX_{B} and LX_{C} are each independently absent (a bond), —CH₂—, —O—, —S—, —S(O)—, —S(O)₂, or —N(R²⁰)—;
wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

3. The compound of claim 2, wherein the linker LI corresponds to formula:
—(CH₂—CH₂—O)ₘ₂—CH₂CH₂-LX_{C}-;
—CH₂C(O)NH—(CH₂—CH₂)ₘ₂—CH₂CH₂-LX_{C}-;
—CH₂C(O)NH—(CH₂—CH₂—O)ₘ₂—CH₂-LX_{C}-;
—CH₂C(O)NH—(CH₂—CH₂—O)ₘ₂—CH₂CH₂-LX_{C}-; or
—CH₂C(O)NH—CH₂—(CH₂—CH₂—O)ₘ₂—CH₂CH₂CH₂-LX_{C}-; wherein —(CH₂—CH₂—O)ₘ₂ or —CH₂C(O)NH or is covalently bound to the PTC or PLM, and LX_{C}- is covalently bound to the PLM or PTC;
m2 is independently 1, 2, 3, 4, 5, or 6;
LX_{C} are each independently absent (a bond), —CH₂—, —O—, —S—, —S(O)—, —S(O)₂—, or —N(R²⁰)—;
wherein each R²⁰ is hydrogen or C₁-C₃ alkyl; and
wherein each —CH₂— in the linker is optionally substituted.

4. The compound of claim 1, wherein the linker LI corresponds to formula
—(CH₂)ₘ₁-LX₁-(CH₂—CH₂-LX₂)ₘ₂—(CH₂)ₘ₃—C(LX₃)-, wherein:
—(CH₂)ₘ₁ is covalently bound to the PTC or PLM, and C(LX₃)- is covalently bound to the PLM or PTC;
each m1, m2, and m3 is independently 0, 1, 2, 3, 4, 5, 6, 7, 8, 9, or 10; and
each LX₁, LX₂, and LX₃ is independently absent (a bond), —O—, —S—, —S(O)—, —S(O)₂—, or —N(R²⁰)—, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

5. The compound of claim 1, wherein the linker LI corresponds to formula —(CH₂)ₘ₁-LX_{B}-(CH₂)ₘ₂-LX_{C}-(CH₂)ₘ₃-LX_{D}-(CH₂)ₘ₄—C(O)—, wherein:
(CH₂)ₘ₁ is covalently bound to the PTC or PLM, and C(O) is covalently bound to the PLM or PTC;
each m1, and m2 is independently 0, 1, 2, 3, 4, 5, 6, 7, 8, 9, 10, 11, or 12;
m3 is independently 1, 2, 3, 4, 5, 6, 7, 8, 9, 10, 11, or 12;
m4 is 0, 1, 2, 3, 4, 5, 6, 7, 8, 9, 10, 11, or 12;
LX_{B}, LX_{C}, and LX_{D} are each independently absent (a bond), —CH₂—, —O—, —S—, —S(O)—, —S(O)₂, or —N(R²⁰)—;
wherein each R²⁰ is independently selected from the group consisting of hydrogen, deuterium, halogen, optionally substituted C₁-C₆ alkyl, optionally substituted aryl, optionally substituted heteroaryl, optionally substituted C₃-C₈ cycloalkyl, and optionally substituted C₃-C₈ heterocyclyl; and
wherein each —CH₂— in the linker is optionally substituted.

6. The compound of claim 5, wherein the Linker corresponds to formula
—(CH₂)ₘ₁-LX_{B}-(CH₂)ₘ₂-LX_{C}-(CH₂)ₘ₃—O—(CH₂)ₘ₄—C(O)—, wherein:
(CH₂)ₘ₁ is covalently bound to the PTC, and C(O) is covalently bound to the PLM;
m1 is 0, 1, 2, or 3;
m2 is independently 0, 1, 2, 3, 4, or 5;
m3 is independently 1, 2, 3, 4, or 5;
m4 is 1, 2 or 3;
LX_{B} and LX_{C} are each independently absent (a bond), —O— or —N(R²⁰)—;
wherein each R²⁰ is independently selected from the group consisting of hydrogen, deuterium, and C₁-C₆ alkyl.

7. The compound of any one of claims 2-6, wherein the sum of m1, m2, and m3 is less than or equal to 24.

8. The compound of any one of claims 2-7, wherein the sum of m1, m2, and m3 is less than or equal to 12.

9. The compound of claim 1, wherein the linker LI is a polyethylene glycol chain ranging in size from about 1 to about 12 ethylene glycol units, wherein each —CH₂— in the polyethylene glycol is optionally substituted.

10. The compound of any one of claims 2-9, wherein the total number of atoms in a straight chain of LI connecting PTC and PLM is 20 or less.

11. The compound of claim 1, wherein the linker LI corresponds to the formula:
-L_{I}-L_{II}(q)-,
wherein:
L_{I} is a bond or a chemical group coupled to at least one of a PLM, a PTC or a combination thereof,
L_{II} is a bond or a chemical group coupled to at least one of a PLM, a PTC, and q is an integer greater than or equal to 0;
wherein each L_{I} and L_{II} is independently selected from a bond, CR^{L1}R^{L2}, —(CH₂)ᵢ—O—, —(CH₂)ᵢ—O—, —O—(CH₂)ᵢ—, —(CH₂)ᵢ—S—, —(CH₂)ᵢ—N—(CH₂)ᵢ—, —S—, —S(O)—, —S(O)₂—, —OP(O)O—(CH₂)ᵢ—, —Si—(CH₂)ᵢ—, NR^{L3} SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁ cycloalkyl optionally substituted with 0-6 R^{L1} and/or R^{L2} groups, C₃₋₁₁ heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups;
wherein i is 1, 2, 3, 4, 5, 6, 7, 8, 9 or 10; and
wherein R^{L1}, R^{L2}, R^{L3}, R^{L4} and R^{L5} are, each independently, H, halo, —C₁₋₈ alkyl, —OC₁₋₈ alkyl, —SC₁₋₈ alkyl, —NHC₁₋₈ alkyl, —N(C₁₋₈ alkyl)₂, —C₃₋₁₁ cycloalkyl, aryl, heteroaryl, —C₃₋₁₁ heterocyclyl, —OC₁₋₈ cycloalkyl, —SC₁₋₈ cycloalkyl, —NHC₁₋₈ cycloalkyl, —N(C₁₋₈ cycloalkyl)₂, —N(C₁₋₈ cycloalkyl)(C₁₋₈ alkyl), —OH, —NH₂, —SH, —SO₂C₁₋₈ alkyl, —P(O)(OC₁₋₈ alkyl)(C₁₋₈ alkyl), —P(O)(OC₁₋₈ alkyl)₂, —C≡C—C₁₋₈ alkyl, —CCH, —CH═CH(C₁₋₈ alkyl), —C(C₁₋₈ alkyl)=CH(C₁₋₈ alkyl), —C(C₁₋₈ alkyl)=C(C₁₋₈ alkyl)₂, —Si(OH)₃, —Si(C₁₋₈ alkyl)₃, —Si(OH)(C₁₋₈ alkyl)₂, —C(═O)C₁₋₈ alkyl, —CO₂H, halogen, —CN, —CF₃, —CHF₂, —CH₂F, —NO₂, —SF₅, —SO₂NHC₁₋₈ alkyl, —SO₂N(C₁₋₈ alkyl)₂, —SONHC₁₋₈ alkyl, —SON(C₁₋₈ alkyl)₂, —CONHC₁₋₈ alkyl, —CON(C₁₋₈ alkyl)₂, —N(C₁₋₈ alkyl)CONH(C₁₋₈ alkyl), —N(C₁₋₈ alkyl)CON(C₁₋₈ alkyl)₂, —NHCONH(C₁₋₈ alkyl), —NHCON(C₁₋₈ alkyl)₂, —NHCONH₂, —N(C₁₋₈ alkyl)SO₂NH(C₁₋₈ alkyl), —N(C₁₋₈ alkyl)SO₂N(C₁₋₈ alkyl)₂, —NHSO₂NH(C₁₋₈ alkyl), —NHSO₂N(C₁₋₈ alkyl)₂, or —NHSO₂NH₂.

12. The compound of claim 1, wherein the linker LI is selected from the group consisting of”
2-(3-(5-(tosyloxy)pentyloxy)propoxy)acetic acid;
2-(3-(3,3-dimethyl-5-(tosyloxy)pentyloxy)propoxy)acetic acid;
2-(3-(3-hydroxy-5-(tosyloxy)pentyloxy)propoxy)acetic acid;
2-(2-(2-(2-(tosyloxy)ethoxy)ethoxy)ethoxy)acetic acid;
2-(2-((2R,3R)-3-(2-(tosyloxy)ethoxy)butan-2-yloxy)ethoxy)acetic acid;
2-(2-((2S,3S)-3-(2-(tosyloxy)ethoxy)butan-2-yloxy)ethoxy)acetic acid;
2-(4-(4-(tosyloxy)butoxy)butoxy)acetic acid;
tert-butyl 2-(3-(4-(tosyloxy)butoxy)propoxy)acetate;
tert-butyl 2-(4-(3-(tosyloxy)propoxy)butoxy)acetate;
tert-butyl 2-(6-(tosyloxy)hexa-2,4-diynyloxy)acetate;
tert-butyl 3-(6-(tosyloxy)hexa-2,4-diynyloxy)propanoate;
tert-butyl 4-(6-(tosyloxy)hexa-2,4-diynyloxy)butanoate;
ethyl 2-(2-(2-aminoethoxy)ethoxy)acetate hydrochloride;
ethyl 2-(5-aminopentyloxy)acetate;
methyl 2-(2-(2-(methylamino)ethoxy)ethoxy)acetate;
ethyl 2-(5-(methylamino)pentyloxy)acetate;
2-(3-(2-(tosyloxy)ethoxy)propoxy)acetic acid;
2-(2-hydroxyethoxy)ethyl 4-methylbenzenesulfonate;
ethyl 2-(2-(2-(tosyloxy)ethoxy)ethoxy)acetate;
ethyl 3-(2-(2-(tosyloxy)ethoxy)ethoxy)propanoate;
ethyl 5-(tosyloxy)pentanoate;
ethyl 3-(2-(tosyloxy)ethoxy)propanoate;
ethyl 2-(5-(tosyloxy)pentyloxy)acetate; ethyl 3-(5-(tosyloxy)pentyloxy)propanoate;
5-hydroxypentyl 4-methylbenzenesulfonate;
ethyl 2-(5-(tosyloxy)pentyloxy)acetate;
ethyl 2-(3-(tosyloxy)propoxy)acetate;
ethyl 2-(2-(tosyloxy)ethoxy)acetate;
ethyl 2-(4-(2-(tosyloxy)ethoxy)butoxy)acetate;
2-(2-(2-hydroxyethoxy)ethoxy)ethyl 4-methylbenzenesulfonate;
2-((2R,3R)-3-(2-hydroxyethoxy)butan-2-yloxy)ethyl 4-methylbenzenesulfonate;
2-(2-piperazin-1-yl)-ethoxy-acetic acid; or
methyl 6-(4-(2-(2-(tert-butoxy)-2-oxoethoxy)ethyl)piperazin-1-yl)nicotinate;
wherein LI is covalently bound to PLM by replacing a hydrogen from LI with a covalent bond to the PLM; and wherein LI is covalently bound to PTC by replacing a hydrogen from LI with a covalent bond the PTC.

13. The compound of claim 1, wherein the linker LI is:

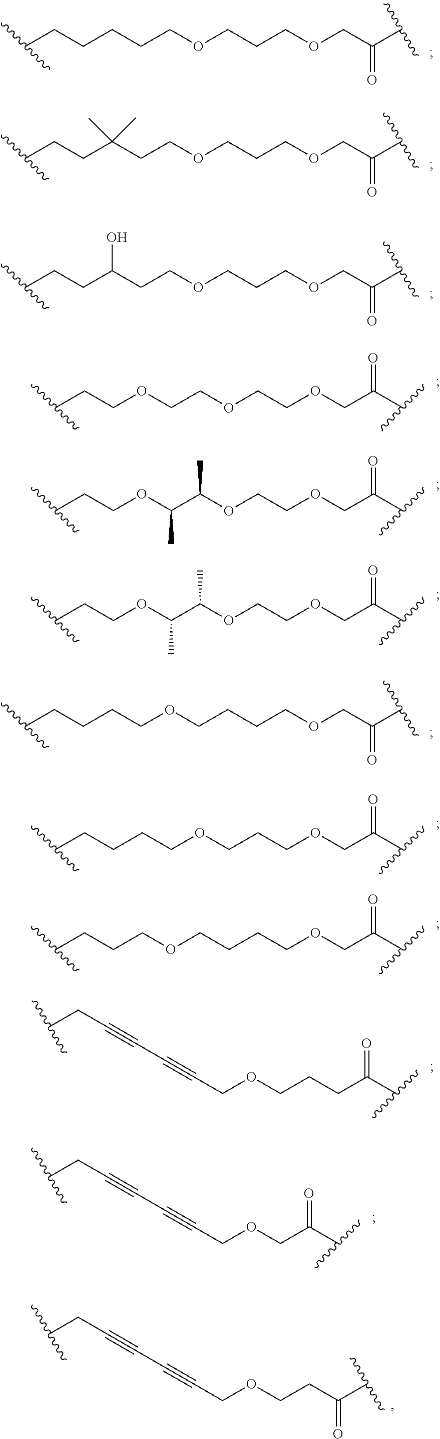

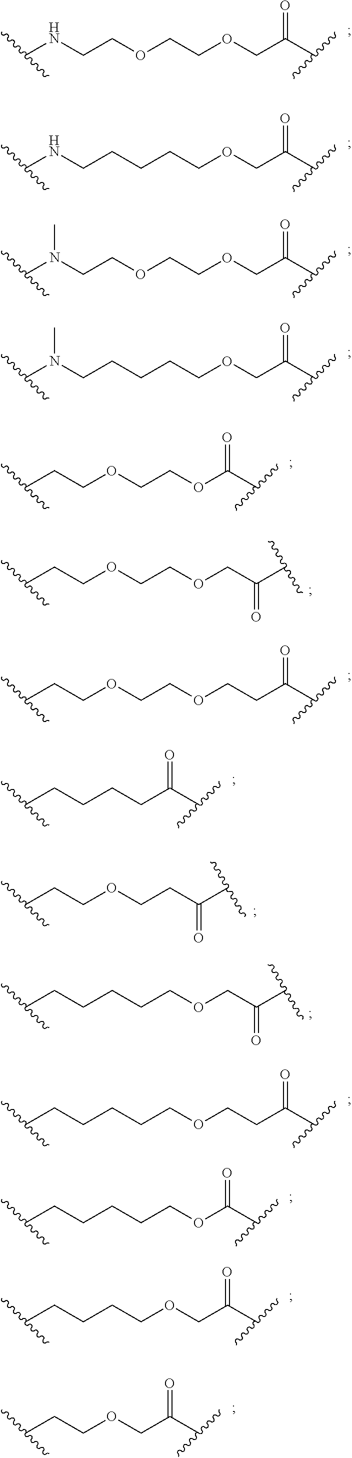

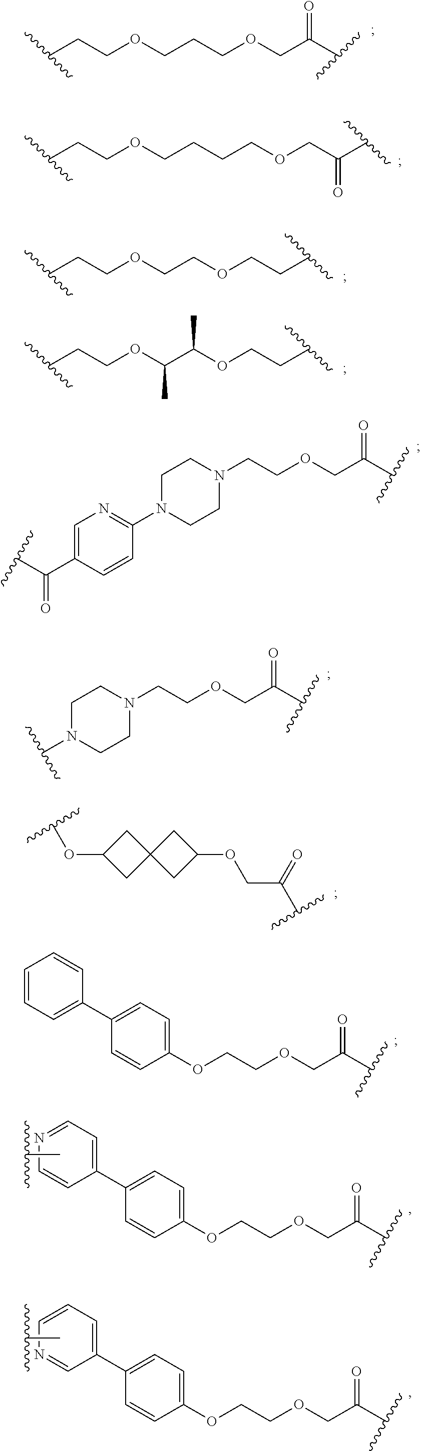

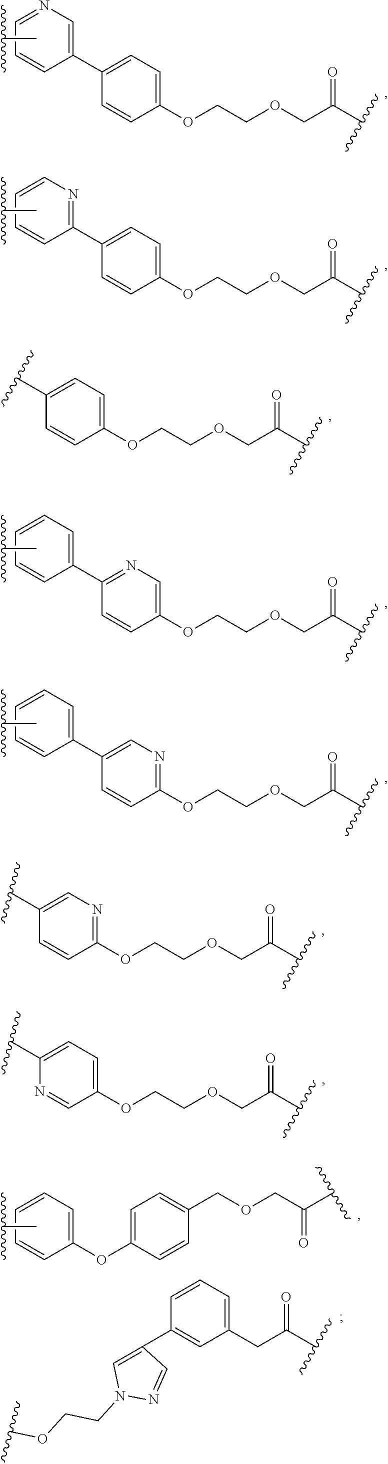

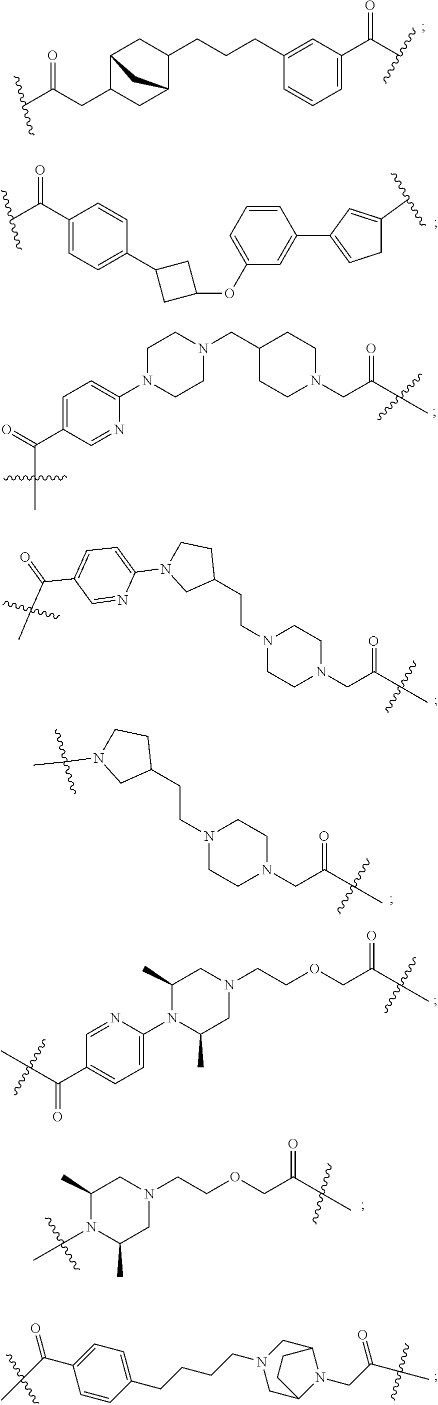

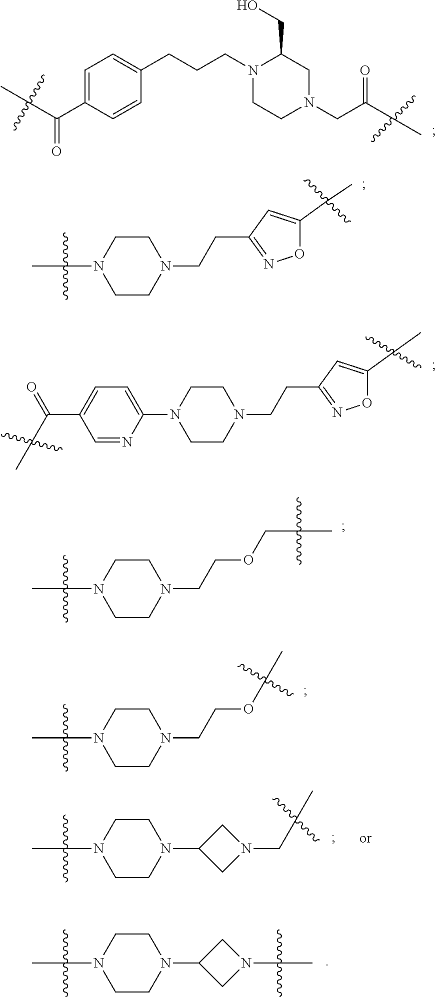

14. The compound of claim 1, wherein the linker LI is selected from:

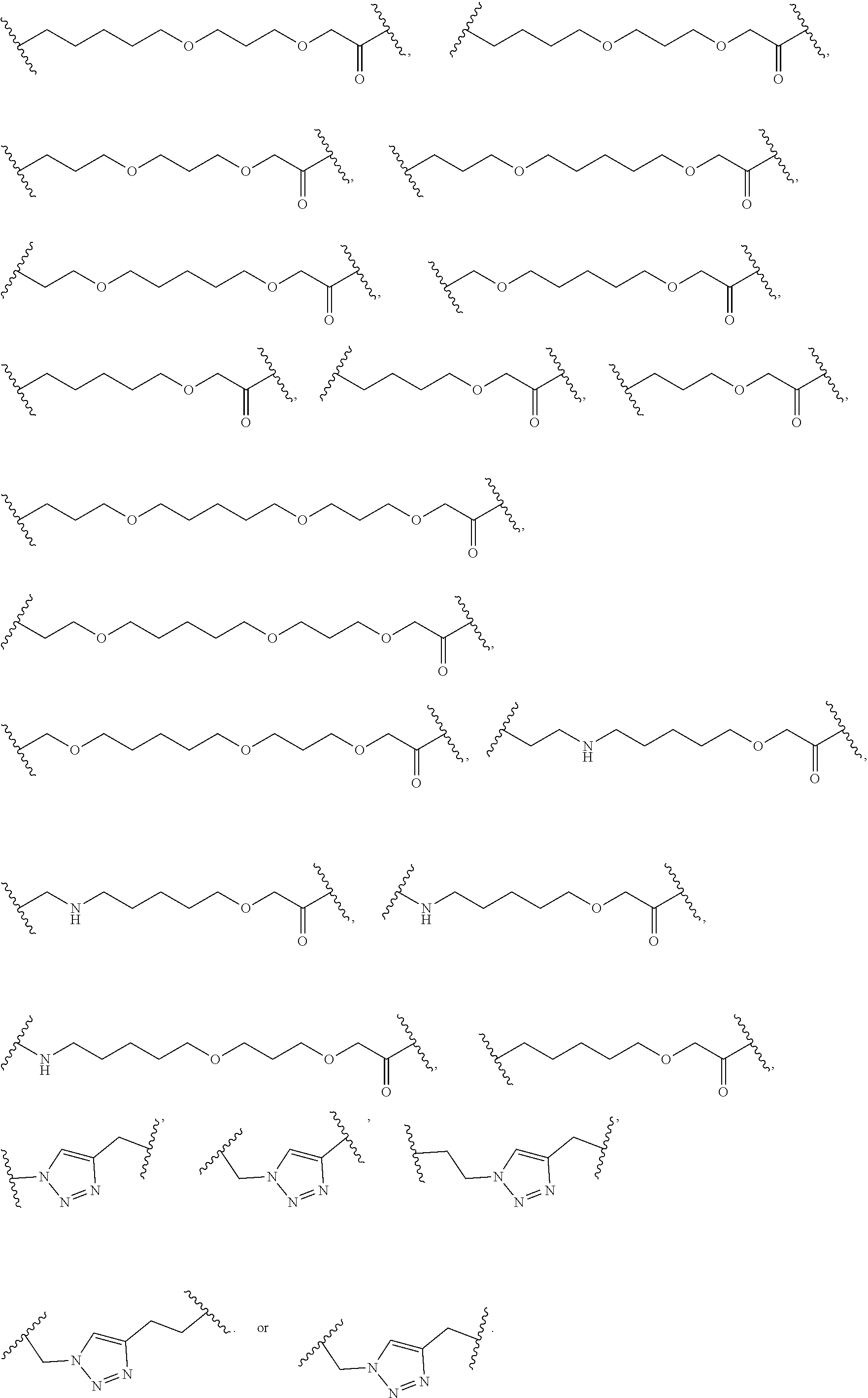

15. The compound of any one of claims 1-14, wherein the PLM is a von Hippel-Lindau (VHL) binding group, an E3 ligase substrate receptor cereblon (CRBN), a mouse double minute 2 homolog (MDM2), or an inhibitor of apoptosis (IAP).

16. The compound of any one of claims 1-15, wherein the PLM is a von Hippel-Lindau (VHL) binding group.

17. The compound of any one of claims 1-16, wherein the PLM has the formula (E3B): 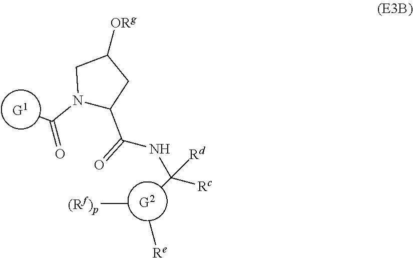
wherein, G¹ is optionally substituted aryl, optionally substituted heteroaryl, or —CR⁹R¹⁰R¹¹;
each R⁹ and R¹⁰ is independently hydrogen, optionally substituted alkyl, optionally substituted cycloalkyl, optionally substituted hydroxyalkyl, optionally substituted heteroaryl, or haloalkyl; or R⁹ and R¹⁰ and the carbon atom to which they are attached form an optionally substituted cycloalkyl;
R¹¹ is optionally substituted heterocyclic, optionally substituted alkoxy, optionally substituted heteroaryl, optionally substituted aryl,
or —NR¹²R¹³, 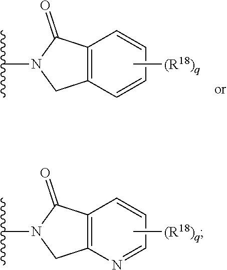
R¹² is H or optionally substituted alkyl;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} and R^{d} is each independently H, haloalkyl, or optionally substituted alkyl;
G² is a phenyl or a 5-10 membered heteroaryl,
R^{e} is H, halogen, CN, OH, NO₂, NR^{c}R^{d}, OR^{c}R, CONR^{c}R^{d}, NR^{c}COR^{d}, SO₂NR^{c}R^{d}, NR^{c}SO₂R^{d}, optionally substituted alkyl, optionally substituted haloalkyl, optionally substituted haloalkoxy; optionally substituted aryl; optionally substituted heteroaryl; optionally substituted cycloalkyl; optionally substituted cycloheteroalkyl;
each R^{f} is independently halo, optionally substituted alkyl, haloalkyl, hydroxy, optionally substituted alkoxy, or haloalkoxy;
R^{g} is H, C₁₋₆ alkyl, —C(O)R¹⁹; —C(O)OR¹⁹; or —C(O)NR¹⁹R¹⁹;
p is 0, 1, 2, 3, or 4;
each R¹⁸ is independently halo, optionally substituted alkoxy, cyano, optionally substituted alkyl, haloalkyl, haloalkoxy or a linker;
each R¹⁹ is independently H, optionally substituted alkyl, or optionally substituted aryl;
q is 0, 1, 2, 3, or 4; and
wherein any one of the hydrogen atoms in the PLM can be replaced to form a covalent bond to the LI.

18. The compound of any one of claims 1-17, wherein the PLM has the formula (E3D): 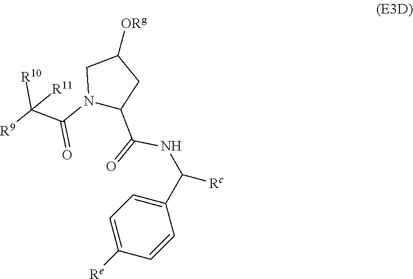
wherein, R⁹ is H;
R¹⁰ is C₁₋₆ alkyl;
R¹¹ is —NR¹²R¹³;
R¹² is H;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R^{c} is H, haloalkyl, methyl, ethyl, isopropyl, cyclopropyl, or C₁-C₆ alkyl (linear, branched, optionally substituted), each optionally substituted with 1 or more halo, hydroxyl, nitro, CN, C₁-C₆ alkyl (linear, branched, optionally substituted), or C₁-C₆ alkoxyl (linear, branched, optionally substituted); and
R^{e} is 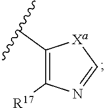
wherein R¹⁷ is H, halo, optionally substituted C₃₋₆cycloalkyl, optionally substituted C₁₋₆alkyl, optionally substituted C₁₋₆alkenyl, or C₁₋₆haloalkyl; and X^{a} is S or O;
R^{g} is H, C₁₋₆ alkyl, —C(O)R¹⁹; —C(O)OR¹⁹; or —C(O)NR¹⁹R¹⁹;
R¹⁹ is independently H, optionally substituted alkyl, or optionally substituted aryl; and
wherein any one of the hydrogen atoms in the PLM can be replaced to form a covalent bond to the LI.

19. The compound of claim 18, wherein the PLM is represented by formula (W-II): 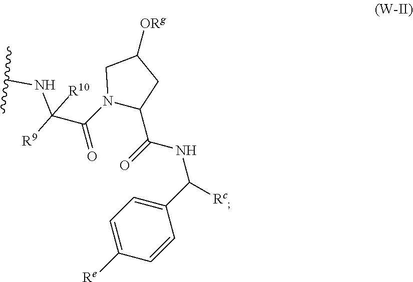
wherein the PLM is covalently bound to the LI via

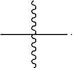

20. The compound of claim 19, wherein the PLM is: 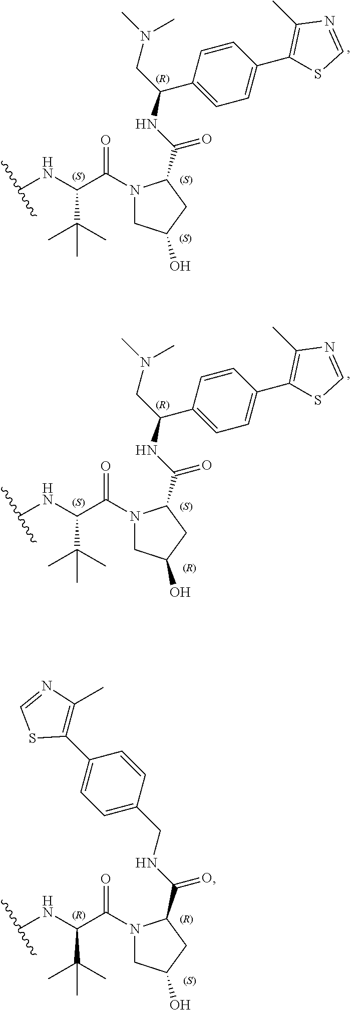 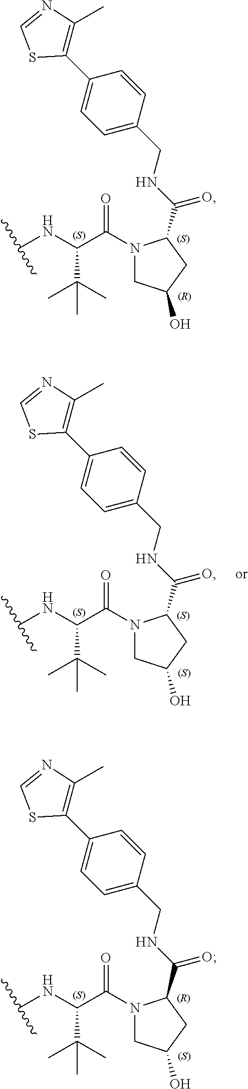
wherein the PLM is covalently bound to the LI via

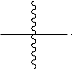

21. The compound of any one of claims 1-16, wherein the PLM is represented by formula (W-IIIA): 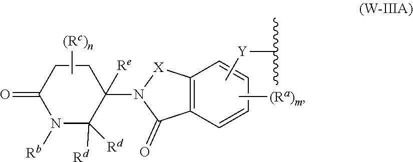
or an enantiomer, diastereomer, stereoisomer, or a pharmaceutically acceptable salt thereof, wherein:
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently halogen, OH, C₁₋₆ alkyl, or C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O), a C₃-C₆ carbocycle, or a 4- to 6-membered heterocycle comprising 1 or 2 heteroatoms selected from N or O;
R^{e} is H, deuterium, C₁₋₃ alkyl, F, or Cl;
m is 0, 1, 2 or 3;
n is 0, 1 or 2; and
wherein the PLM is covalently bound to the LI via

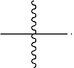

22. The compound of claim 21, wherein the PLM is represented by formula (W-IIIB): 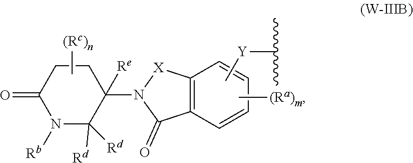
or an enantiomer, diastereomer, stereoisomer, or a pharmaceutically acceptable salt thereof, wherein: 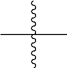
represent a bond to the LI;
Y is a bond, —(CH₂)₁₋₆—, —(CH₂)₀₋₆—O—, —(CH₂)₀₋₆—C(O)NR^{g}—, —(CH₂)₀₋₆—NR^{g}C(O)—, —(CH₂)₀₋₆—NH— or —(CH₂)₀₋₆—NR^{f} or;
X is —C(O)— or —C(R^{b})₂—;
each R^{a} is independently C₁₋₆ alkoxy;
R^{f} is C₁₋₆ alkyl, —C(O)(C₁₋₆ alkyl), or —C(O)(C₃₋₆ cycloalkyl);
R^{g} is H or C₁₋₆ alkyl;
R^{b} is H or C₁₋₃ alkyl;
R^{c} is each independently C₁₋₃ alkyl;
R^{d} is each independently H or C₁₋₃ alkyl; or two R^{d}, together with the carbon atom to which they are attached, form a C(O) or a C₃-C₆ carbocycle;
R^{e} is H, deuterium, C₁₋₃ alkyl, F, or Cl;
m is 0, 1, 2 or 3;
n is 0, 1 or 2; and
wherein the PLM is covalently bound to the LI via

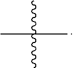

23. The compound of claim 21 or 22, wherein X is —C(C₁₋₃ alkyl)₂.

24. The compound of any one of claims 21-23, wherein the PLM is selected from the group consisting of: 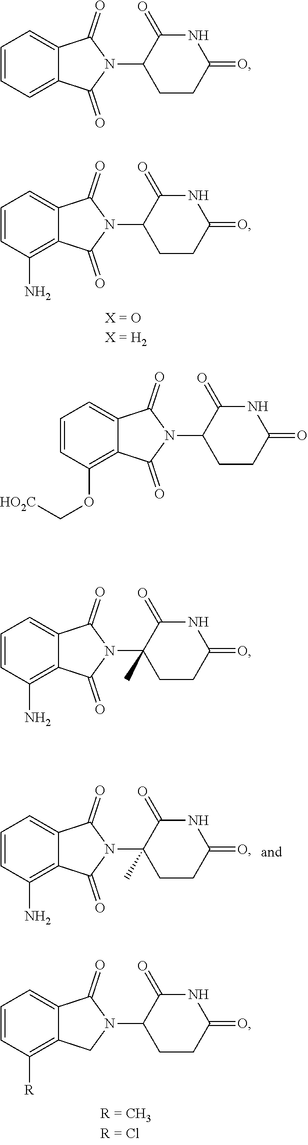
wherein any one of the hydrogen atoms in the PLM can be replaced to form a covalent bond to the LI.

25. The compound of any one of claims 21-23, wherein the PLM is:

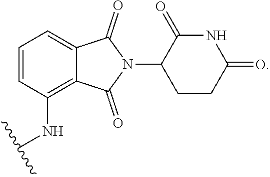 [SMILES: *Nc1cccc2c1C(=O)N(C1CCC(=O)NC1=O)C2=O]

26. The compound of any one of claims 1-16, wherein the PLM is represented by: 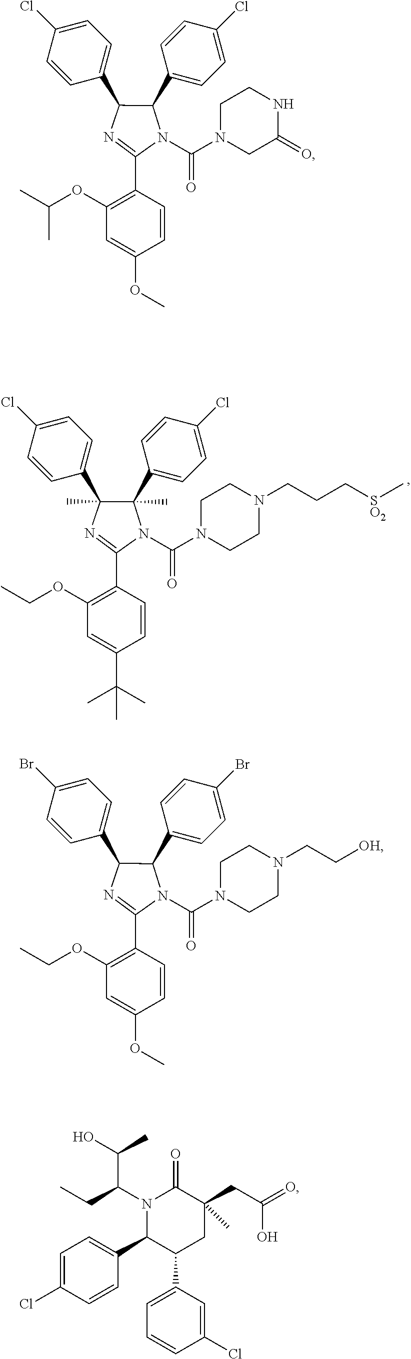 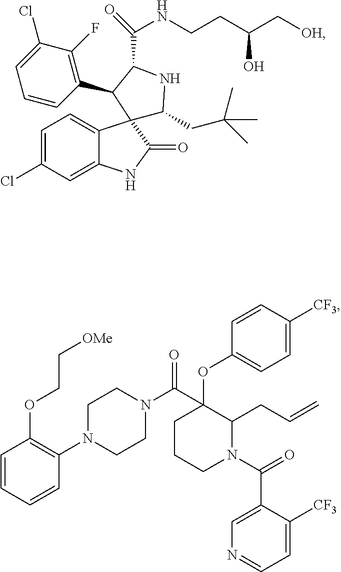 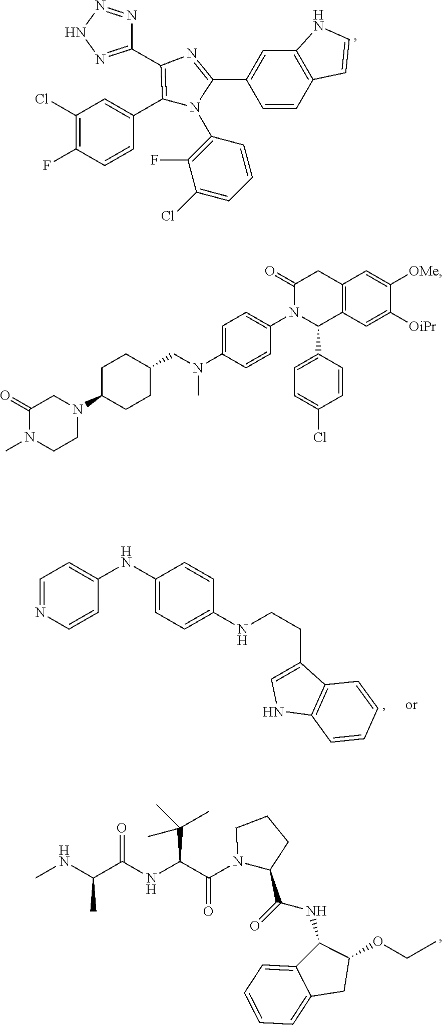 [SMILES: Fc1ccc(-c2c(-c3nn[nH]n3)nc(-c3ccc4cc[nH]c4c3)n2-c2cccc(Cl)c2F)cc1Cl ; COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2 ; c1ccc2c(CCNc3ccc(Nc4ccncc4)cc3)c[nH]c2c1 ; CCO[C@@H]1Cc2ccccc2[C@@H]1NC(=O)[C@@H]1CCCN1C(=O)[C@@H](NC(=O)[C@@H](C)NC)C(C)(C)C]
wherein any one of the hydrogen atoms in the PLM can be replaced to form a covalent bond to the LI.

27. The compound of any one of claims 1-16, wherein the PLM is

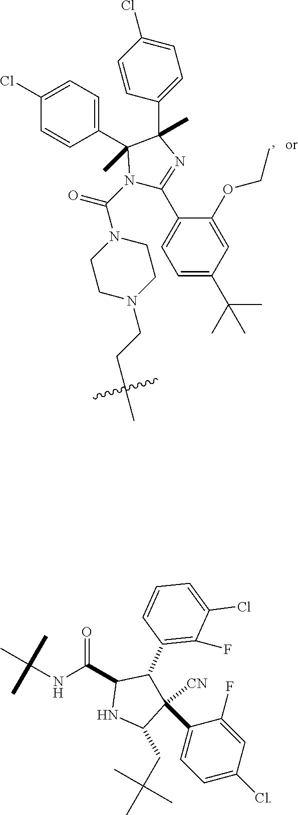

28. The compound of any one of claims 1-27, wherein the PTC has the structure of formula (IVA): 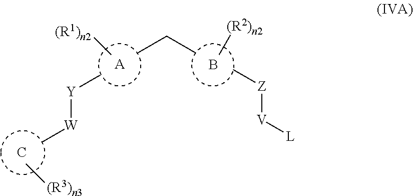
or a pharmaceutically acceptable salt, tautomer, stereoisomer or prodrug thereof, wherein:
A and B are each independently selected from phenyl, pyridyl, pyrimidyl, or thiophene;
C is a 3- to 10-membered ring;
X is a bond, —(CR⁵R⁶)ₜ—, or —NR⁷;
Y and Z are each independently a bond, —CH₂—, —C(CH₃)H—, —O—, —S—, —NH—, —NCH₃—, or —N(COCH₃)—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, or optionally substituted —(C₁-C₆ alkyl)-SO₂R¹⁶;
R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR⁴SO₂R¹⁶, —NR¹⁴COR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is H or C₁-C₆ alkyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form a 3- to 6-membered heterocyclyl;
R¹⁶ is hydrogen, C₁-C₃ alkyl, C₂-C₃ alkenyl, or C₂-C₃ alkynyl;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

29. The compound of claim 28, wherein C is 5- to 10-membered heteroaryl or aryl.

30. The compound of claim 28 or 29, wherein C is 5- to 7-membered heteroaryl comprising 1, 2, or 3 heteroatoms selected from O, S, or N as a ring member.

31. The compound of any one of claims 28-30, wherein C, which is substituted with (R³)n3, is pyrazole, imidazole, oxazole, oxadiazole, oxazolone, isoxazole, thiazole, pyridyl, pyrazine, furan or pyrimidyl.

32. The compound of any one of claims 28-30, wherein C, which is substituted with (R³)n3, is selected from the group consisting of: 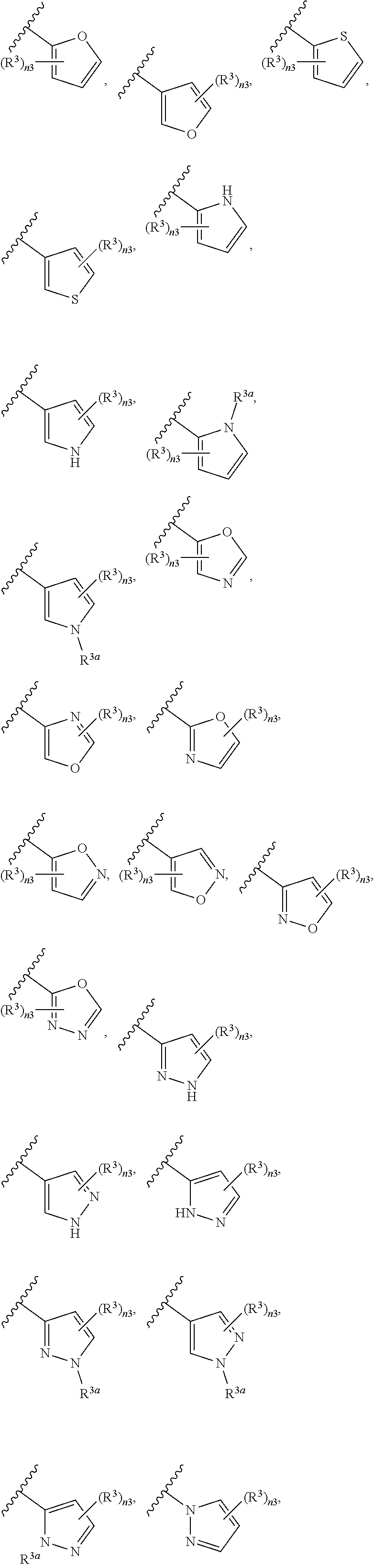 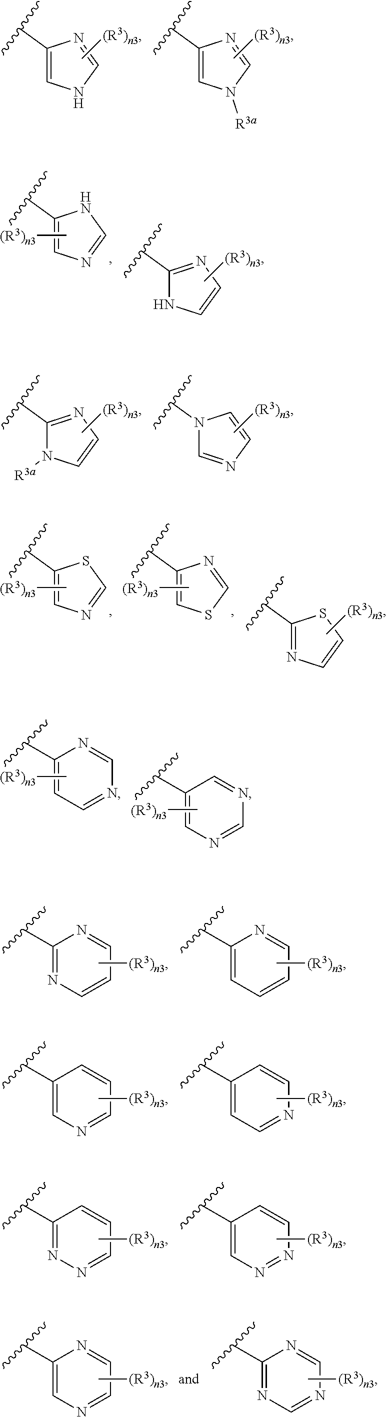
wherein R^{3a} is C₁-C₃ alkyl.

33. The compound of any one of claims 28-32, wherein R¹ and R² are each independently Cl, —CN, —CF₃, —OH, methyl, methoxy, or —CONH₂.

34. The compound of any one of claims 28-33, wherein:
A and B are phenyl;
X is —(CR⁵R⁶)ₜ—;
Y and Z are each —O—;
V is —CH₂— or —CH₂CH₂—;
L is halogen;
R¹ and R² are each independently hydrogen, halogen, —CN, —CF₃, —OH, or optionally substituted C₁-C₆ alkyl;
R⁵ and R⁶ are each independently hydrogen, halogen, —OH, or C₁-C₃ alkyl; and
R¹⁶ is hydrogen, C₁-C₃ alkyl, C₂-C₃ alkenyl, or C₂-C₃ alkynyl.

35. The compound of claim 34, wherein:
R⁵ and R⁶ are each independently hydrogen, or C₁-C₃ alkyl;
W is —CH₂— or —C(CH₃)H—;
V is —CH₂CH₂—; and
R¹ and R² are each independently hydrogen, halogen, or —CN.

36. The compound of claim 28, wherein the PTC has the structure of formula (A-I): 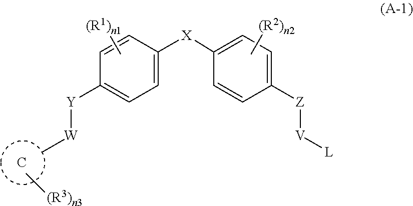
or a pharmaceutically acceptable salt, tautomer, stereoisomer or prodrug thereof, wherein:
C is a 5- to 7-membered monocyclic heteroaryl comprising 1, 2, or 3 heteroatoms selected from O, S, or N as a ring member;
X is a bond, —(CR⁵R⁶)ₜ—, or —NR⁷;
Y is a bond, —CH₂—, —C(CH₃)H—, —O—, —S—, —NH—, —NCH₃—, or —N(COCH₃)—;
Z is a bond, —CH₂—, —O—, or —NH—;
W is a bond, —CH₂—, —C(CH₃)H—, —C(═O)—, —N(R⁷)CO—, or —CONR⁷—;
V is —CH₂— and L is halogen, —NH₂, or —CF₃; or
V is —CH₂CH₂— and L is halogen or —NH₂;
R¹ and R² are each independently hydrogen, halogen, —CN, —CF₃, methyl, or —CONH₂;
R³ is selected from —CN, C₁-C₃ alkoxy, —CF₃, C₁-C₃ alkyl, C₂-C₃ alkenyl, C₂-C₃ alkynyl, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen, halogen, —OH, or C₁-C₃ alkyl;
R⁷ is H or C₁-C₆ alkyl;
n1 and n2 are each independently 0, 1, or 2;
n3 is 1, 2, 3, 4 or 5;
t is 0, 1 or 2; and
wherein one atom or one chemical group in the PTC is replaced to form a covalent bond to the LI.

37. The compound of any one of claims 28-36, wherein: at least one R³ is selected from the group consisting of —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), and —N(CH₃)COO(C₁-C₃ alkyl).

38. The compound of claim 36, wherein:
X is a bond or —(CR⁵R⁶)ₜ;
W is a bond, —CH₂—, or —C(CH₃)H—;
Y is —O—;
Z is —O—;
V is —CH₂— or —CH₂CH₂—; and
L is halogen.

39. The compound of claim 28, wherein the PTC has the structure of formula (G-II) 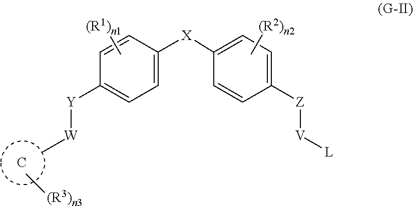
or a pharmaceutically acceptable salt, tautomer, stereoisomer or prodrug thereof, wherein:
C is 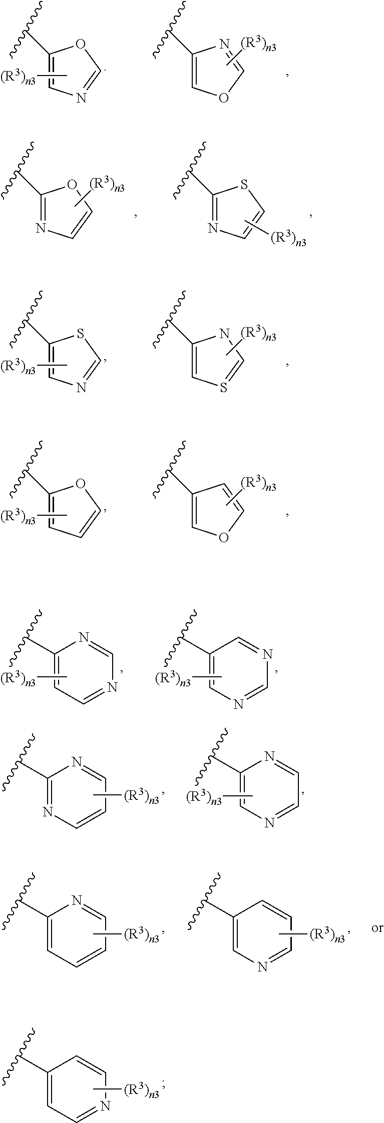
X is —(CR⁵R⁶)ₜ—;
Y is —O—;
Z is —O—;
W is —CH₂— or —C(CH₃)H—;
V is —CH₂CH₂—;
L is halogen;
R¹ and R² are each independently Cl or —CN;
at least one R³ is selected from —CN, C₁-C₃ alkoxy, —CONH₂, —NHSO₂CH₃, —N(CH₃)SO₂CH₃, —NHSO₂CH₂CH₃, —N(CH₃)SO₂CH₂CH₃, or —SO₂CH₃ and the other R³, if present, is selected from —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
R⁵ and R⁶ are each independently hydrogen or methyl;
n1 and n2 are each independently 0, 1, or 2;
n3 is 1 or 2;
t is 1; and
wherein one atom or one chemical group in the PTC is replaced to form a covalent bond to the LI.

40. The compound of claim 39, wherein: at least one R³ is selected from the group consisting of —NHSO₂CH₃, —NHSO₂CH₂CH₃, or —SO₂CH₃ and the other R³, if present, is selected from —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), and —N(CH₃)COO(C₁-C₃ alkyl).

41. The compound of any one of claims 1-40 wherein an atom in L is replaced with a covalent bond to the LI.

42. The compound of claim 4, wherein a halogen is replaced with a covalent bond to the LI

43. The compound of any one of claims 1-40, wherein an atom in ring C, R¹, or R³, is replaced with a covalent bond to the LI.

44. The compound of claim 4, wherein a hydrogen atom is replaced with a covalent bond to the LI

45. The compound of claim 1, wherein the PTC is selected from the group consisting of: 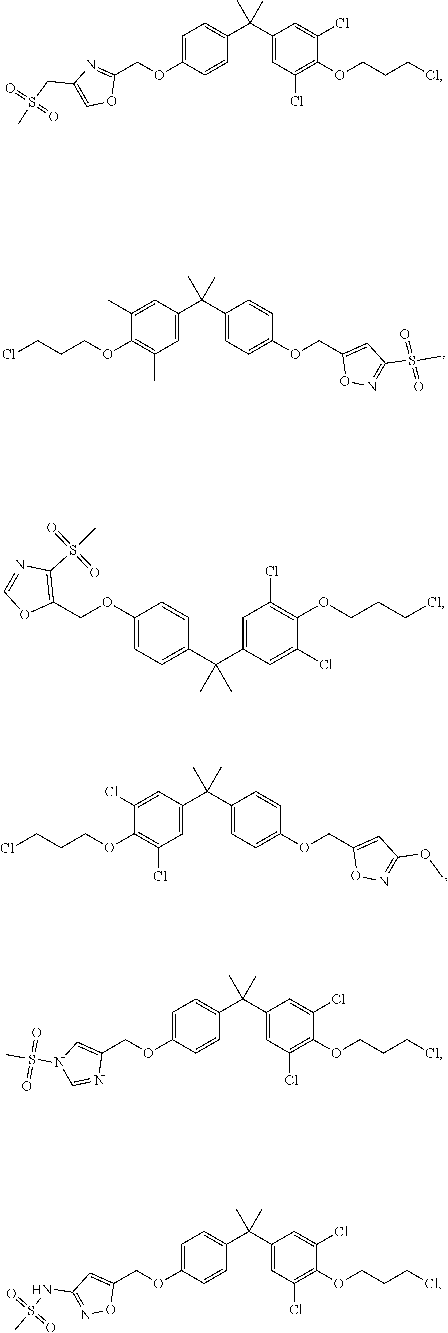 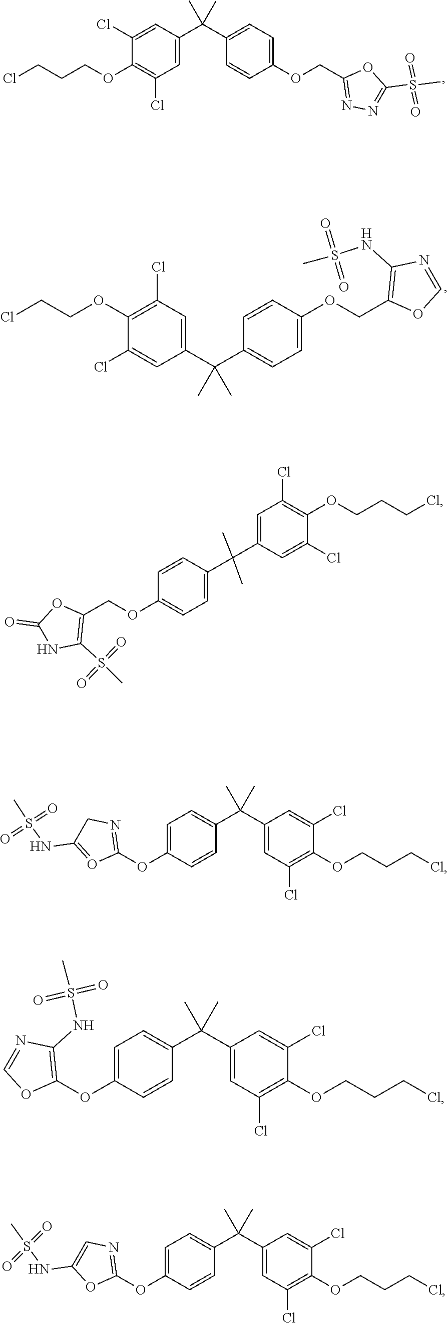 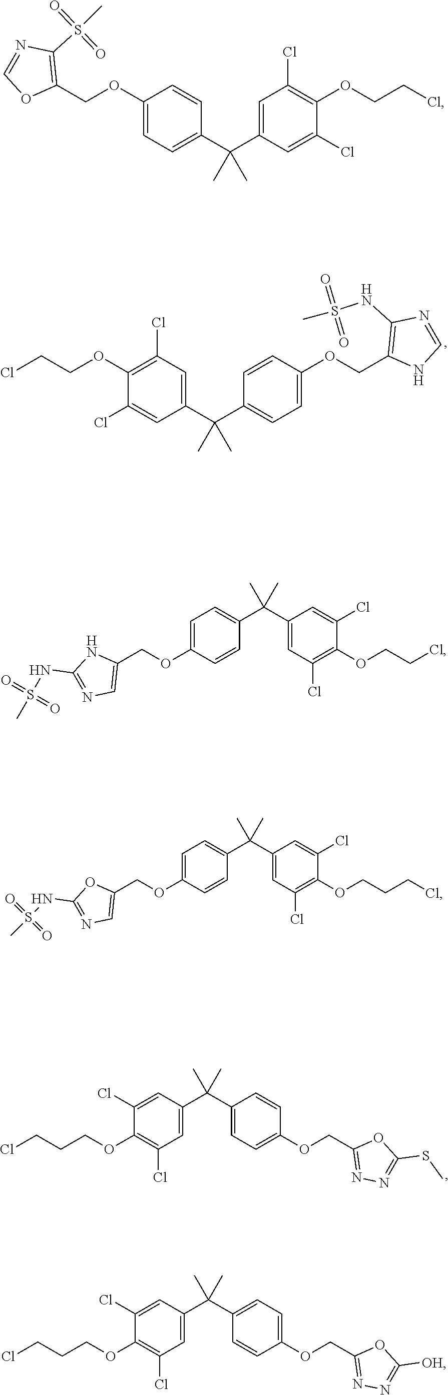 [SMILES: CC(C)(c1ccc(OCc2ocnc2S(C)(=O)=O)cc1)c1cc(Cl)c(OCCCl)c(Cl)c1 ; CC(C)(c1ccc(OCc2[nH]cnc2NS(C)(=O)=O)cc1)c1cc(Cl)c(OCCCl)c(Cl)c1 ; CC(C)(c1ccc(OCc2cnc(NS(C)(=O)=O)[nH]2)cc1)c1cc(Cl)c(OCCCl)c(Cl)c1 ; CC(C)(c1ccc(OCc2cnc(NS(C)(=O)=O)o2)cc1)c1cc(Cl)c(OCCCCl)c(Cl)c1 ; CSc1nnc(COc2ccc(C(C)(C)c3cc(Cl)c(OCCCCl)c(Cl)c3)cc2)o1 ; CC(C)(c1ccc(OCc2nnc(O)o2)cc1)c1cc(Cl)c(OCCCCl)c(Cl)c1] 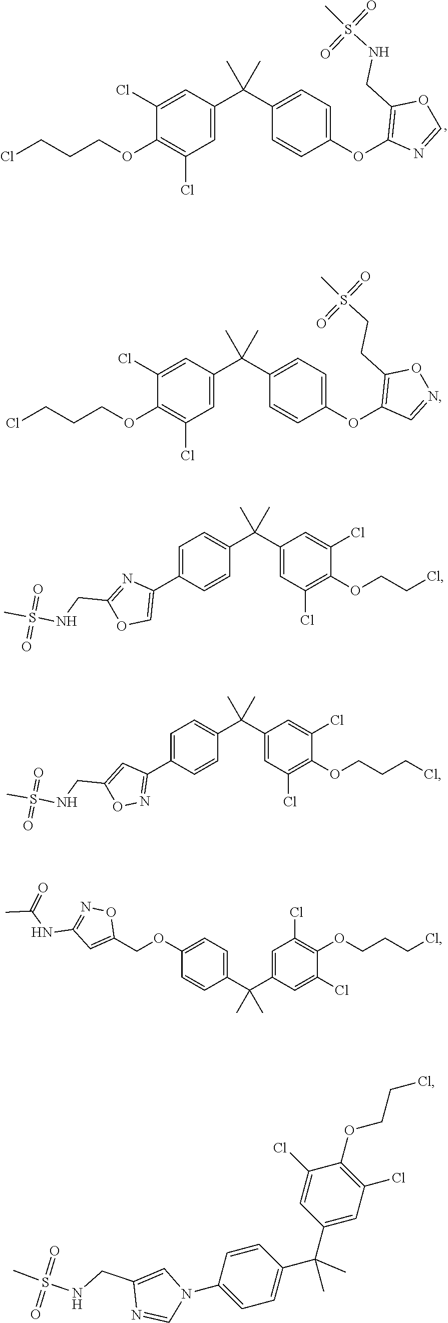 [SMILES: CC(C)(c1ccc(Oc2ncoc2CNS(C)(=O)=O)cc1)c1cc(Cl)c(OCCCCl)c(Cl)c1 ; CC(C)(c1ccc(Oc2cnoc2CCS(C)(=O)=O)cc1)c1cc(Cl)c(OCCCCl)c(Cl)c1 ; CC(C)(c1ccc(-c2coc(CNS(C)(=O)=O)n2)cc1)c1cc(Cl)c(OCCCl)c(Cl)c1 ; CC(C)(c1ccc(-c2cc(CNS(C)(=O)=O)on2)cc1)c1cc(Cl)c(OCCCCl)c(Cl)c1 ; CC(=O)Nc1cc(COc2ccc(C(C)(C)c3cc(Cl)c(OCCCCl)c(Cl)c3)cc2)on1 ; CC(C)(c1ccc(-n2cnc(CNS(C)(=O)=O)c2)cc1)c1cc(Cl)c(OCCCl)c(Cl)c1] 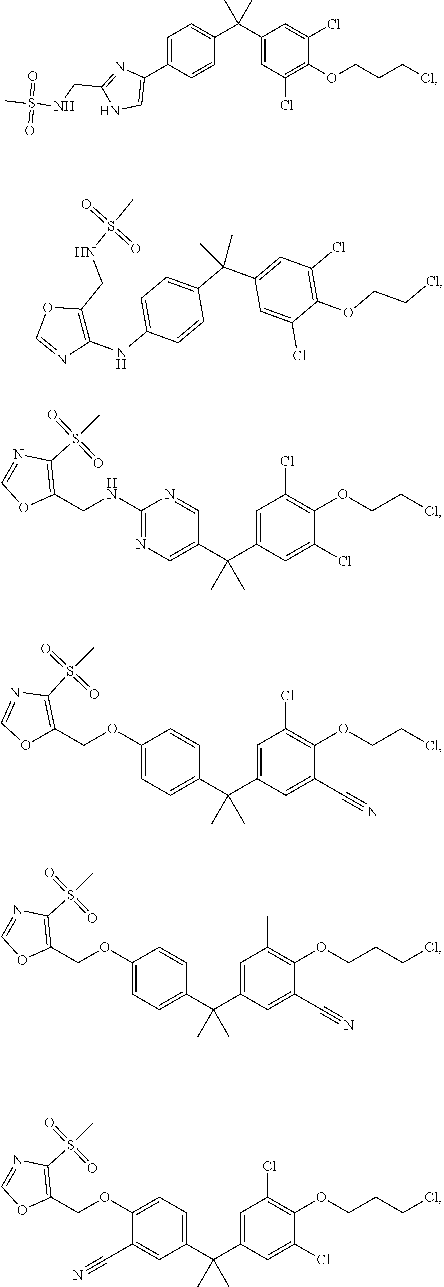 [SMILES: CC(C)(c1ccc(-c2c[nH]c(CNS(C)(=O)=O)n2)cc1)c1cc(Cl)c(OCCCCl)c(Cl)c1 ; CC(C)(c1ccc(Nc2ncoc2CNS(C)(=O)=O)cc1)c1cc(Cl)c(OCCCl)c(Cl)c1 ; CC(C)(c1cnc(NCc2ocnc2S(C)(=O)=O)nc1)c1cc(Cl)c(OCCCl)c(Cl)c1 ; CC(C)(c1ccc(OCc2ocnc2S(C)(=O)=O)cc1)c1cc(Cl)c(OCCCl)c(C#N)c1 ; Cc1cc(C(C)(C)c2ccc(OCc3ocnc3S(C)(=O)=O)cc2)cc(C#N)c1OCCCCl ; CC(C)(c1cc(Cl)c(OCCCCl)c(Cl)c1)c1ccc(OCc2ocnc2S(C)(=O)=O)c(C#N)c1] 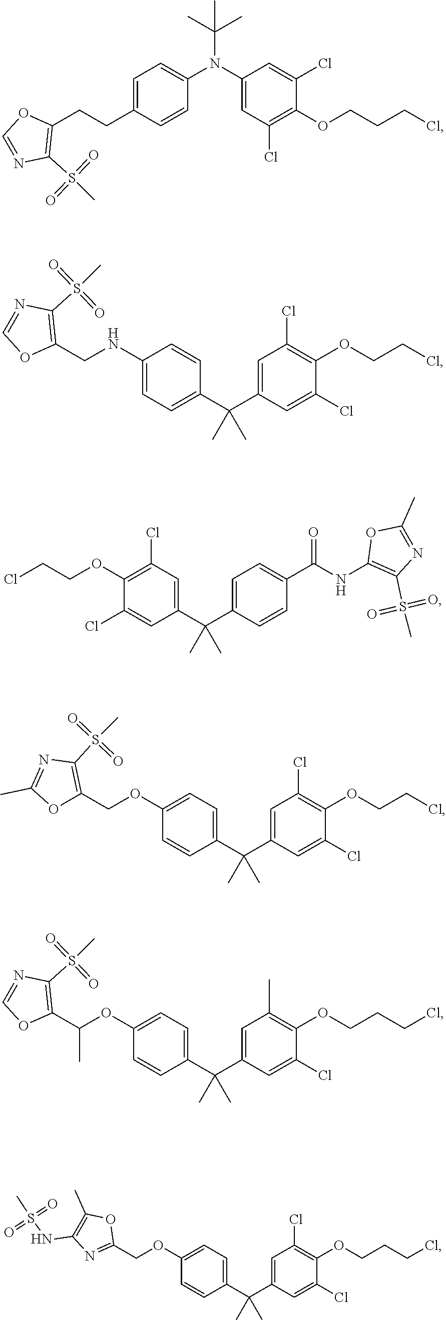 [SMILES: CC(C)(C)N(c1ccc(CCc2ocnc2S(C)(=O)=O)cc1)c1cc(Cl)c(OCCCCl)c(Cl)c1 ; CC(C)(c1ccc(NCc2ocnc2S(C)(=O)=O)cc1)c1cc(Cl)c(OCCCl)c(Cl)c1 ; Cc1nc(S(C)(=O)=O)c(NC(=O)c2ccc(C(C)(C)c3cc(Cl)c(OCCCl)c(Cl)c3)cc2)o1 ; Cc1nc(S(C)(=O)=O)c(COc2ccc(C(C)(C)c3cc(Cl)c(OCCCl)c(Cl)c3)cc2)o1 ; Cc1cc(C(C)(C)c2ccc(OC(C)c3ocnc3S(C)(=O)=O)cc2)cc(Cl)c1OCCCCl ; Cc1oc(COc2ccc(C(C)(C)c3cc(Cl)c(OCCCCl)c(Cl)c3)cc2)nc1NS(C)(=O)=O] 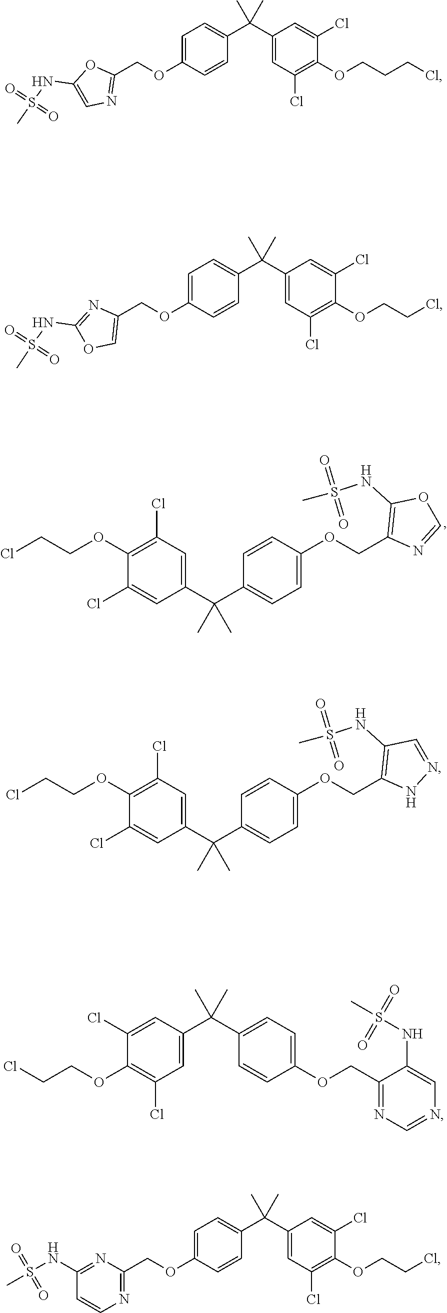 [SMILES: CC(C)(c1ccc(OCc2ncc(NS(C)(=O)=O)o2)cc1)c1cc(Cl)c(OCCCCl)c(Cl)c1 ; CC(C)(c1ccc(OCc2coc(NS(C)(=O)=O)n2)cc1)c1cc(Cl)c(OCCCl)c(Cl)c1 ; CC(C)(c1ccc(OCc2ncoc2NS(C)(=O)=O)cc1)c1cc(Cl)c(OCCCl)c(Cl)c1 ; CC(C)(c1ccc(OCc2[nH]ncc2NS(C)(=O)=O)cc1)c1cc(Cl)c(OCCCl)c(Cl)c1 ; CC(C)(c1ccc(OCc2ncncc2NS(C)(=O)=O)cc1)c1cc(Cl)c(OCCCl)c(Cl)c1 ; CC(C)(c1ccc(OCc2nccc(NS(C)(=O)=O)n2)cc1)c1cc(Cl)c(OCCCl)c(Cl)c1] 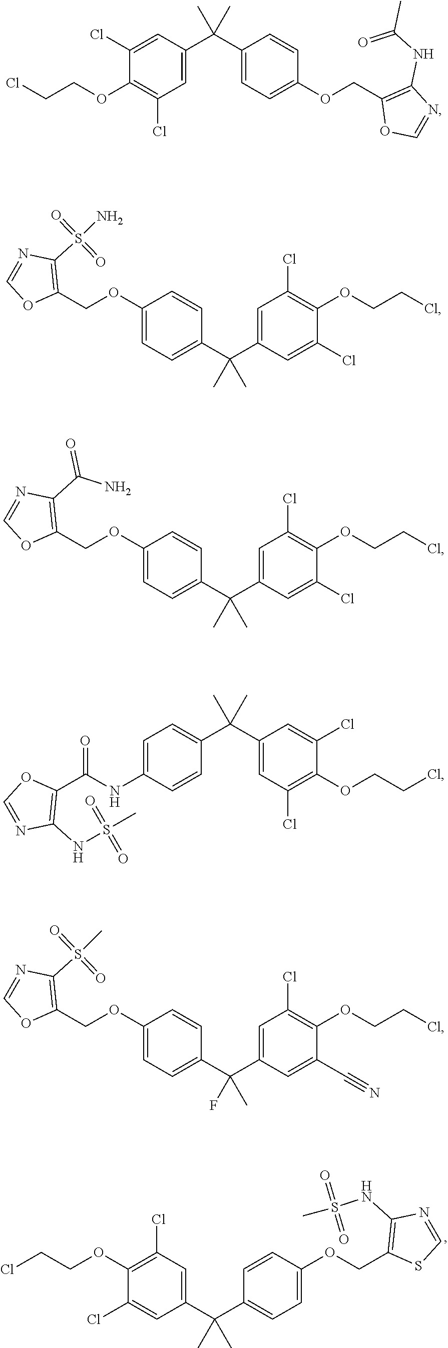 [SMILES: CC(=O)Nc1ncoc1COc1ccc(C(C)(C)c2cc(Cl)c(OCCCl)c(Cl)c2)cc1 ; CC(C)(c1ccc(OCc2ocnc2S(N)(=O)=O)cc1)c1cc(Cl)c(OCCCl)c(Cl)c1 ; CC(C)(c1ccc(OCc2ocnc2C(N)=O)cc1)c1cc(Cl)c(OCCCl)c(Cl)c1 ; CC(C)(c1ccc(NC(=O)c2ocnc2NS(C)(=O)=O)cc1)c1cc(Cl)c(OCCCl)c(Cl)c1 ; CC(F)(c1ccc(OCc2ocnc2S(C)(=O)=O)cc1)c1cc(Cl)c(OCCCl)c(C#N)c1 ; CC(C)(c1ccc(OCc2scnc2NS(C)(=O)=O)cc1)c1cc(Cl)c(OCCCl)c(Cl)c1] 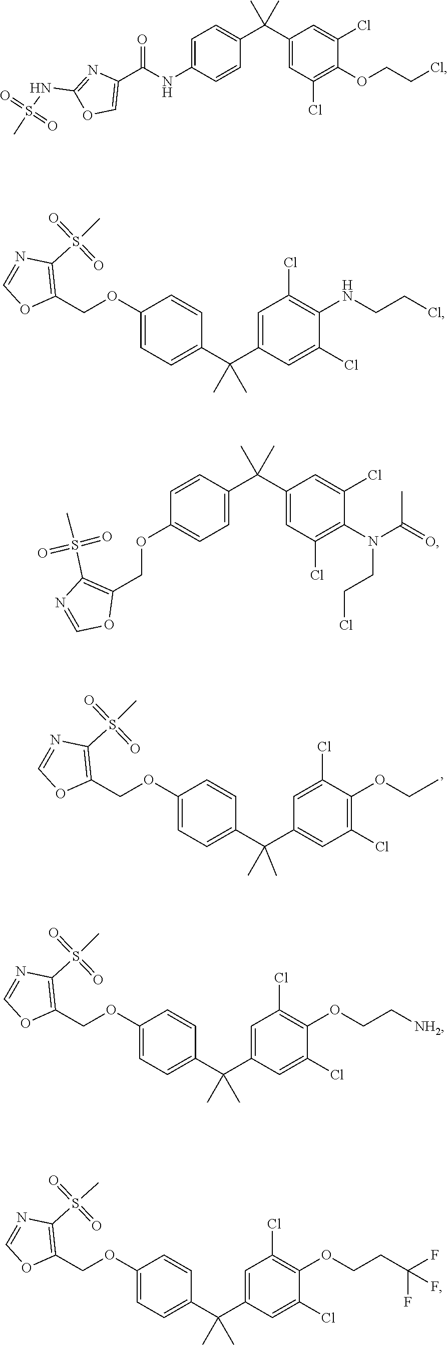 [SMILES: CC(C)(c1ccc(NC(=O)c2coc(NS(C)(=O)=O)n2)cc1)c1cc(Cl)c(OCCCl)c(Cl)c1 ; CC(C)(c1ccc(OCc2ocnc2S(C)(=O)=O)cc1)c1cc(Cl)c(NCCCl)c(Cl)c1 ; CC(=O)N(CCCl)c1c(Cl)cc(C(C)(C)c2ccc(OCc3ocnc3S(C)(=O)=O)cc2)cc1Cl ; CCOc1c(Cl)cc(C(C)(C)c2ccc(OCc3ocnc3S(C)(=O)=O)cc2)cc1Cl ; CC(C)(c1ccc(OCc2ocnc2S(C)(=O)=O)cc1)c1cc(Cl)c(OCCN)c(Cl)c1 ; CC(C)(c1ccc(OCc2ocnc2S(C)(=O)=O)cc1)c1cc(Cl)c(OCCC(F)(F)F)c(Cl)c1] 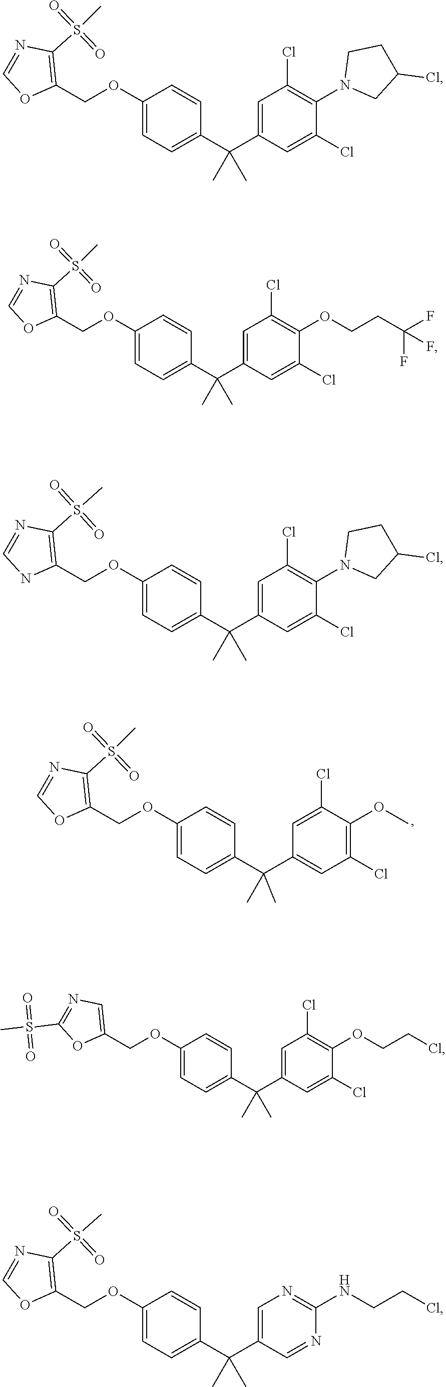 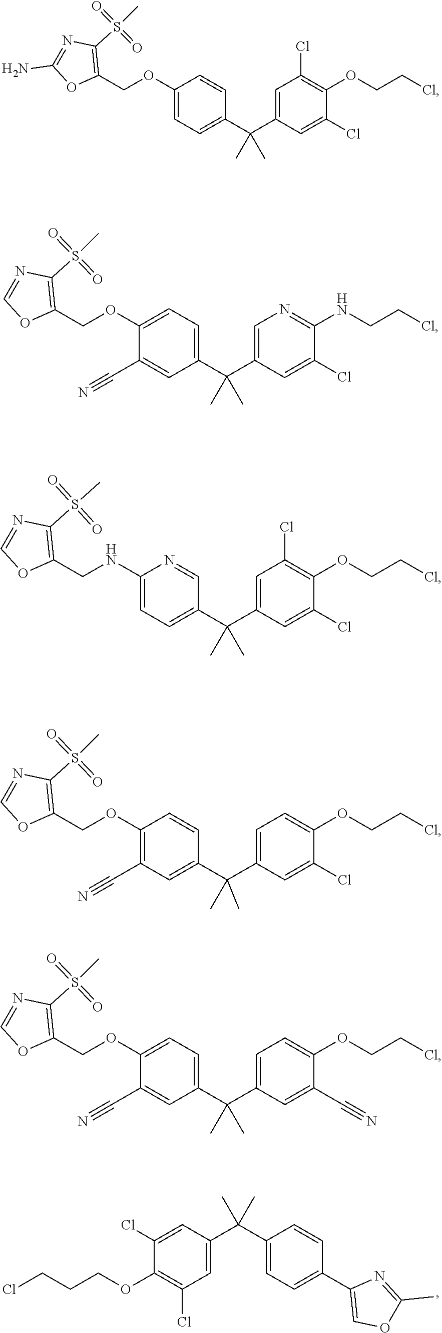 [SMILES: CC(C)(c1ccc(OCc2oc(N)nc2S(C)(=O)=O)cc1)c1cc(Cl)c(OCCCl)c(Cl)c1 ; CC(C)(c1cnc(NCCCl)c(Cl)c1)c1ccc(OCc2ocnc2S(C)(=O)=O)c(C#N)c1 ; CC(C)(c1ccc(NCc2ocnc2S(C)(=O)=O)nc1)c1cc(Cl)c(OCCCl)c(Cl)c1 ; CC(C)(c1ccc(OCCCl)c(Cl)c1)c1ccc(OCc2ocnc2S(C)(=O)=O)c(C#N)c1 ; CC(C)(c1ccc(OCCCl)c(C#N)c1)c1ccc(OCc2ocnc2S(C)(=O)=O)c(C#N)c1 ; Cc1nc(-c2ccc(C(C)(C)c3cc(Cl)c(OCCCCl)c(Cl)c3)cc2)co1] 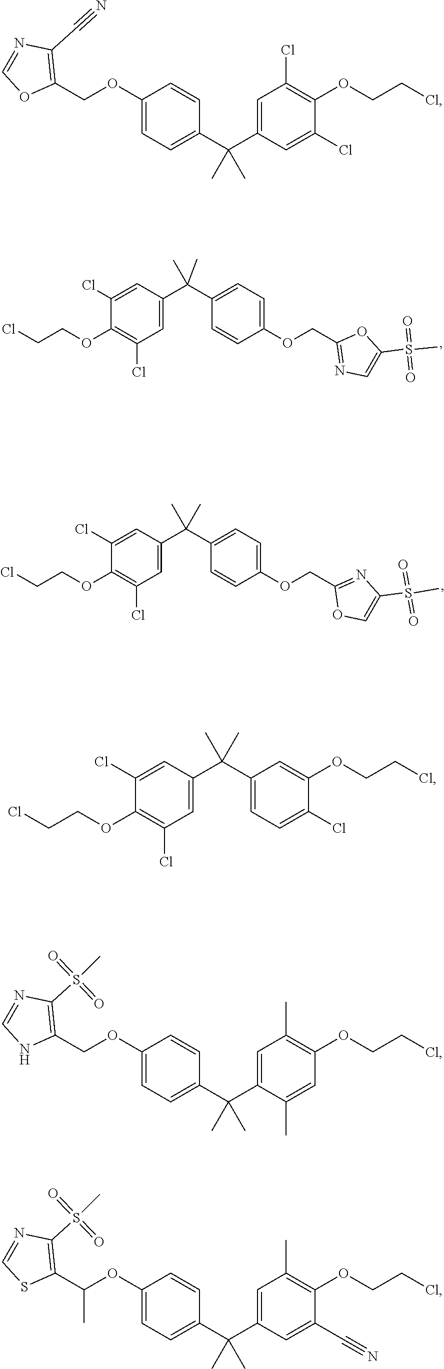 [SMILES: CC(C)(c1ccc(OCc2ocnc2C#N)cc1)c1cc(Cl)c(OCCCl)c(Cl)c1 ; CC(C)(c1ccc(OCc2ncc(S(C)(=O)=O)o2)cc1)c1cc(Cl)c(OCCCl)c(Cl)c1 ; CC(C)(c1ccc(OCc2nc(S(C)(=O)=O)co2)cc1)c1cc(Cl)c(OCCCl)c(Cl)c1 ; CC(C)(c1cc(Cl)c(OCCCl)c(Cl)c1)c1ccc(Cl)c(OCCCl)c1 ; Cc1cc(C(C)(C)c2ccc(OCc3[nH]cnc3S(C)(=O)=O)cc2)c(C)cc1OCCCl ; Cc1cc(C(C)(C)c2ccc(OC(C)c3scnc3S(C)(=O)=O)cc2)cc(C#N)c1OCCCl] 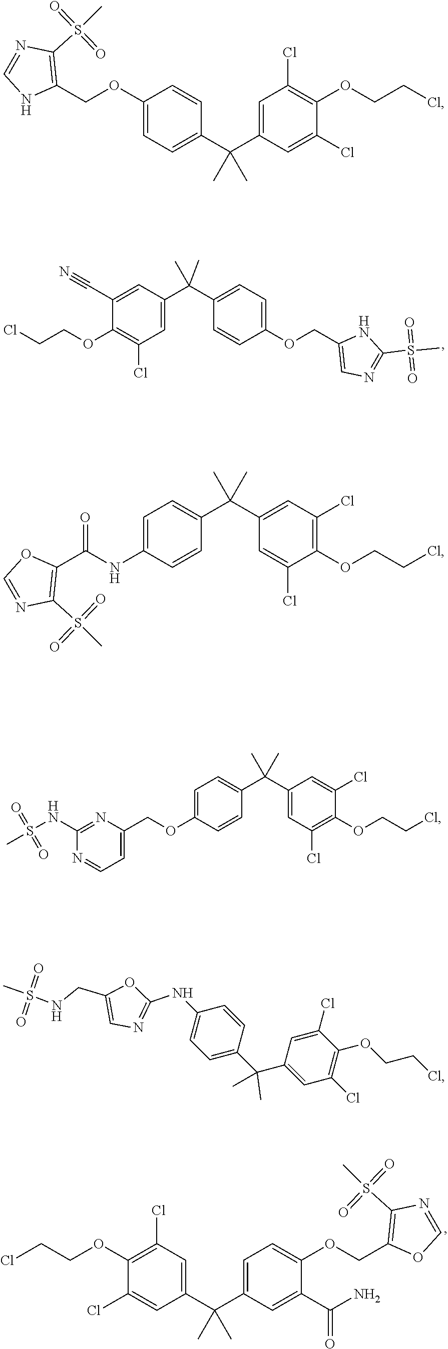 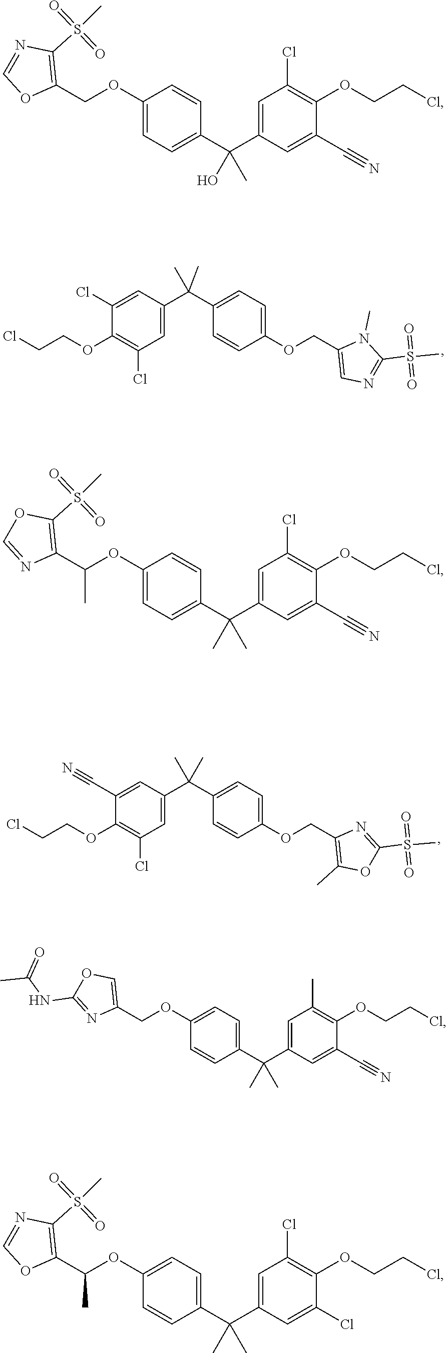 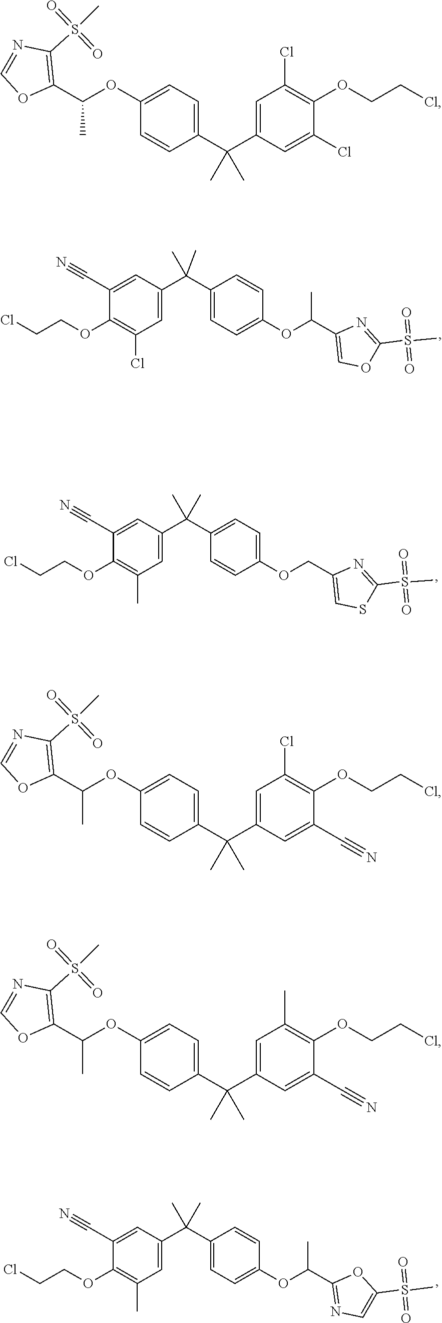 [SMILES: C[C@@H](Oc1ccc(C(C)(C)c2cc(Cl)c(OCCCl)c(Cl)c2)cc1)c1ocnc1S(C)(=O)=O ; CC(Oc1ccc(C(C)(C)c2cc(Cl)c(OCCCl)c(C#N)c2)cc1)c1coc(S(C)(=O)=O)n1 ; Cc1cc(C(C)(C)c2ccc(OCc3csc(S(C)(=O)=O)n3)cc2)cc(C#N)c1OCCCl ; CC(Oc1ccc(C(C)(C)c2cc(Cl)c(OCCCl)c(C#N)c2)cc1)c1ocnc1S(C)(=O)=O ; Cc1cc(C(C)(C)c2ccc(OC(C)c3ocnc3S(C)(=O)=O)cc2)cc(C#N)c1OCCCl ; Cc1cc(C(C)(C)c2ccc(OC(C)c3ncc(S(C)(=O)=O)o3)cc2)cc(C#N)c1OCCCl] 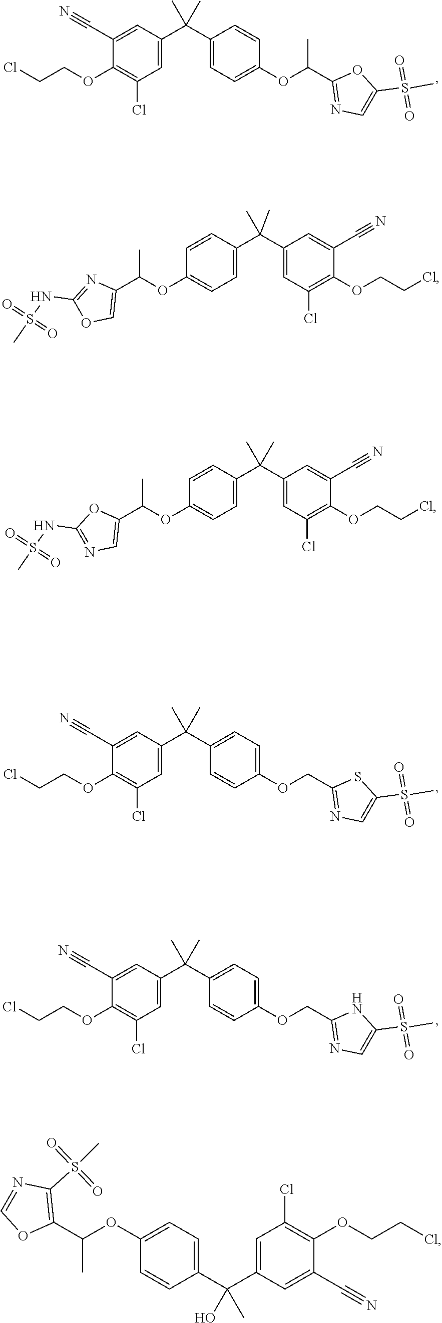 [SMILES: CC(Oc1ccc(C(C)(C)c2cc(Cl)c(OCCCl)c(C#N)c2)cc1)c1ncc(S(C)(=O)=O)o1 ; CC(Oc1ccc(C(C)(C)c2cc(Cl)c(OCCCl)c(C#N)c2)cc1)c1coc(NS(C)(=O)=O)n1 ; CC(Oc1ccc(C(C)(C)c2cc(Cl)c(OCCCl)c(C#N)c2)cc1)c1cnc(NS(C)(=O)=O)o1 ; CC(C)(c1ccc(OCc2ncc(S(C)(=O)=O)s2)cc1)c1cc(Cl)c(OCCCl)c(C#N)c1 ; CC(C)(c1ccc(OCc2ncc(S(C)(=O)=O)[nH]2)cc1)c1cc(Cl)c(OCCCl)c(C#N)c1 ; CC(Oc1ccc(C(C)(O)c2cc(Cl)c(OCCCl)c(C#N)c2)cc1)c1ocnc1S(C)(=O)=O] 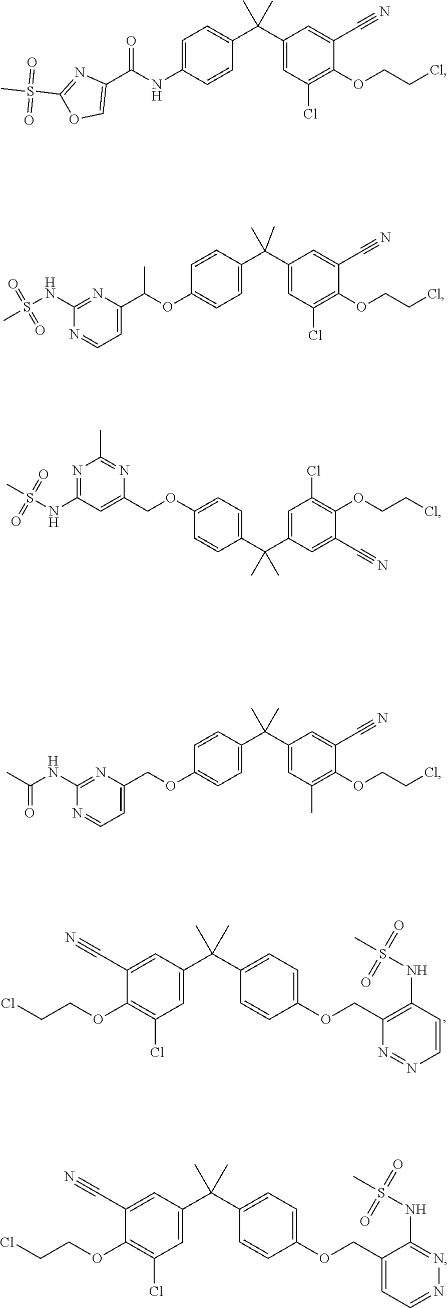 [SMILES: CC(C)(c1ccc(NC(=O)c2coc(S(C)(=O)=O)n2)cc1)c1cc(Cl)c(OCCCl)c(C#N)c1 ; CC(Oc1ccc(C(C)(C)c2cc(Cl)c(OCCCl)c(C#N)c2)cc1)c1ccnc(NS(C)(=O)=O)n1 ; Cc1nc(COc2ccc(C(C)(C)c3cc(Cl)c(OCCCl)c(C#N)c3)cc2)cc(NS(C)(=O)=O)n1 ; CC(=O)Nc1nccc(COc2ccc(C(C)(C)c3cc(C)c(OCCCl)c(C#N)c3)cc2)n1 ; CC(C)(c1ccc(OCc2nnccc2NS(C)(=O)=O)cc1)c1cc(Cl)c(OCCCl)c(C#N)c1 ; CC(C)(c1ccc(OCc2ccnnc2NS(C)(=O)=O)cc1)c1cc(Cl)c(OCCCl)c(C#N)c1] 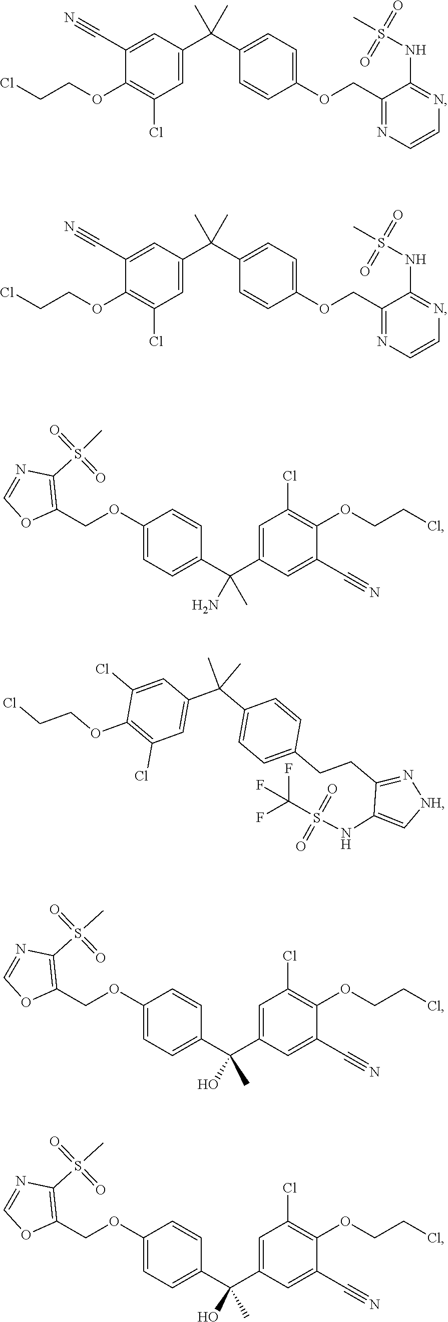 [SMILES: CC(C)(c1ccc(OCc2nccnc2NS(C)(=O)=O)cc1)c1cc(Cl)c(OCCCl)c(C#N)c1 ; CC(C)(c1ccc(OCc2nccnc2NS(C)(=O)=O)cc1)c1cc(Cl)c(OCCCl)c(C#N)c1 ; CC(N)(c1ccc(OCc2ocnc2S(C)(=O)=O)cc1)c1cc(Cl)c(OCCCl)c(C#N)c1 ; CC(C)(c1ccc(CCc2n[nH]cc2NS(=O)(=O)C(F)(F)F)cc1)c1cc(Cl)c(OCCCl)c(Cl)c1 ; C[C@@](O)(c1ccc(OCc2ocnc2S(C)(=O)=O)cc1)c1cc(Cl)c(OCCCl)c(C#N)c1 ; C[C@](O)(c1ccc(OCc2ocnc2S(C)(=O)=O)cc1)c1cc(Cl)c(OCCCl)c(C#N)c1] 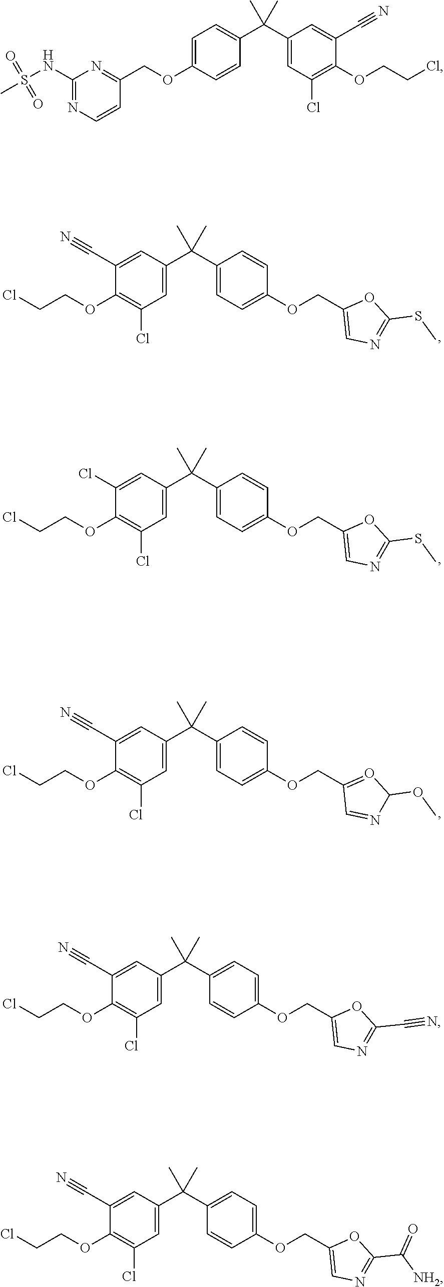 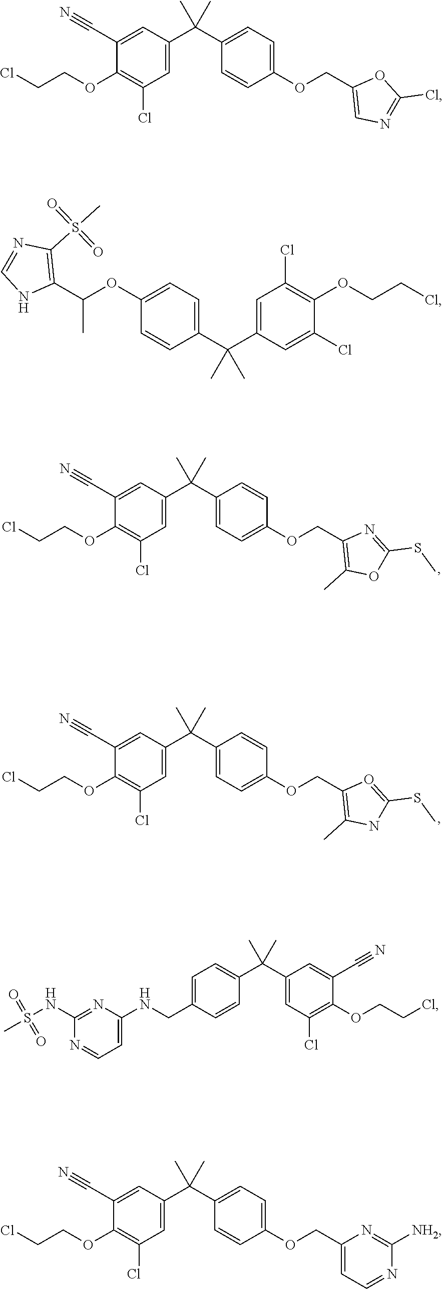 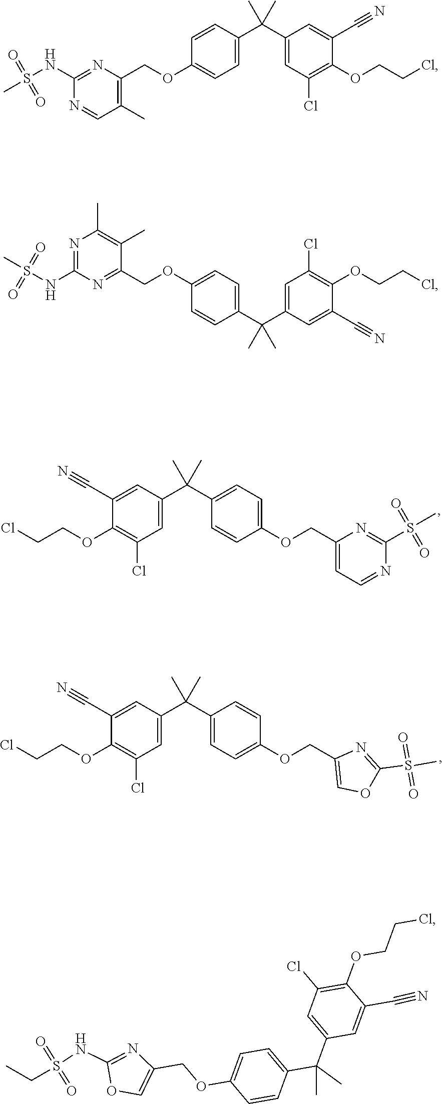 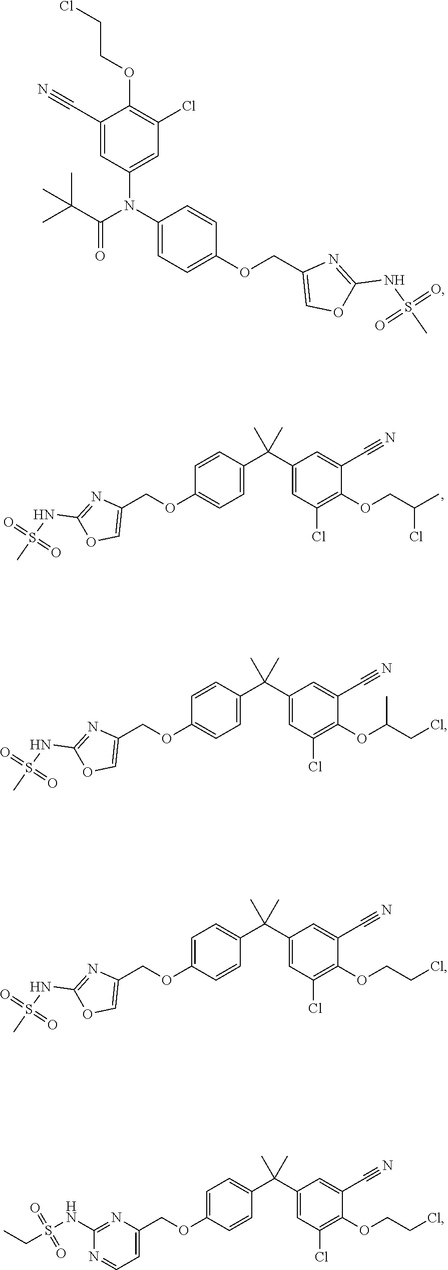 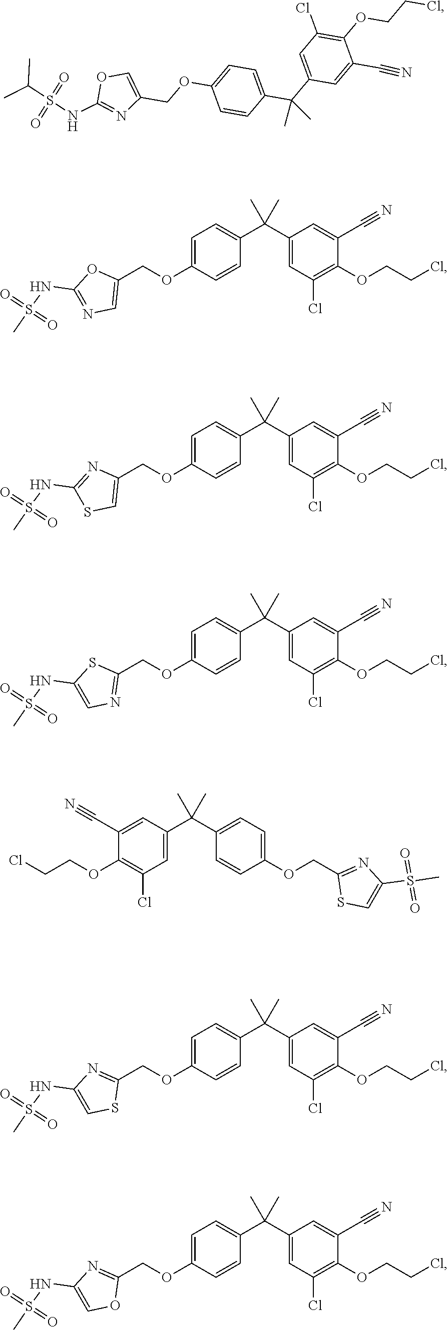 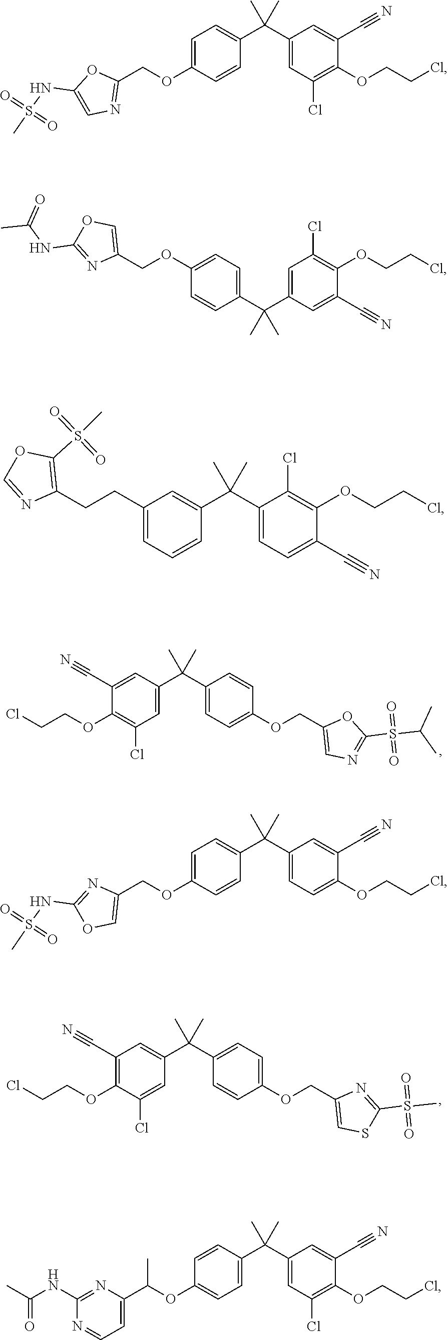 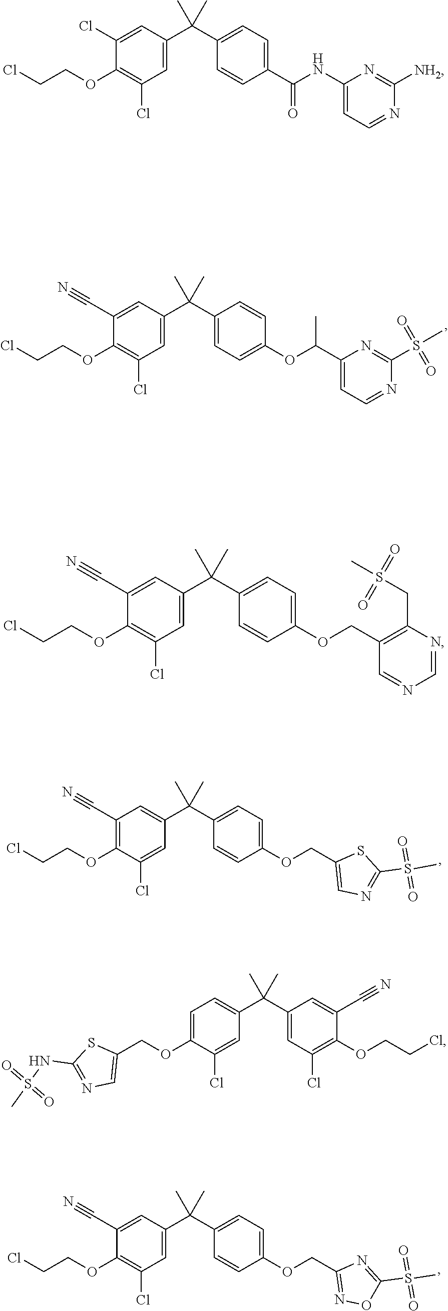 [SMILES: CC(C)(c1ccc(C(=O)Nc2ccnc(N)n2)cc1)c1cc(Cl)c(OCCCl)c(Cl)c1 ; CC(Oc1ccc(C(C)(C)c2cc(Cl)c(OCCCl)c(C#N)c2)cc1)c1ccnc(S(C)(=O)=O)n1 ; CC(C)(c1ccc(OCc2cncnc2CS(C)(=O)=O)cc1)c1cc(Cl)c(OCCCl)c(C#N)c1 ; CC(C)(c1ccc(OCc2cnc(S(C)(=O)=O)s2)cc1)c1cc(Cl)c(OCCCl)c(C#N)c1 ; CC(C)(c1ccc(OCc2cnc(NS(C)(=O)=O)s2)c(Cl)c1)c1cc(Cl)c(OCCCl)c(C#N)c1 ; CC(C)(c1ccc(OCc2noc(S(C)(=O)=O)n2)cc1)c1cc(Cl)c(OCCCl)c(C#N)c1] 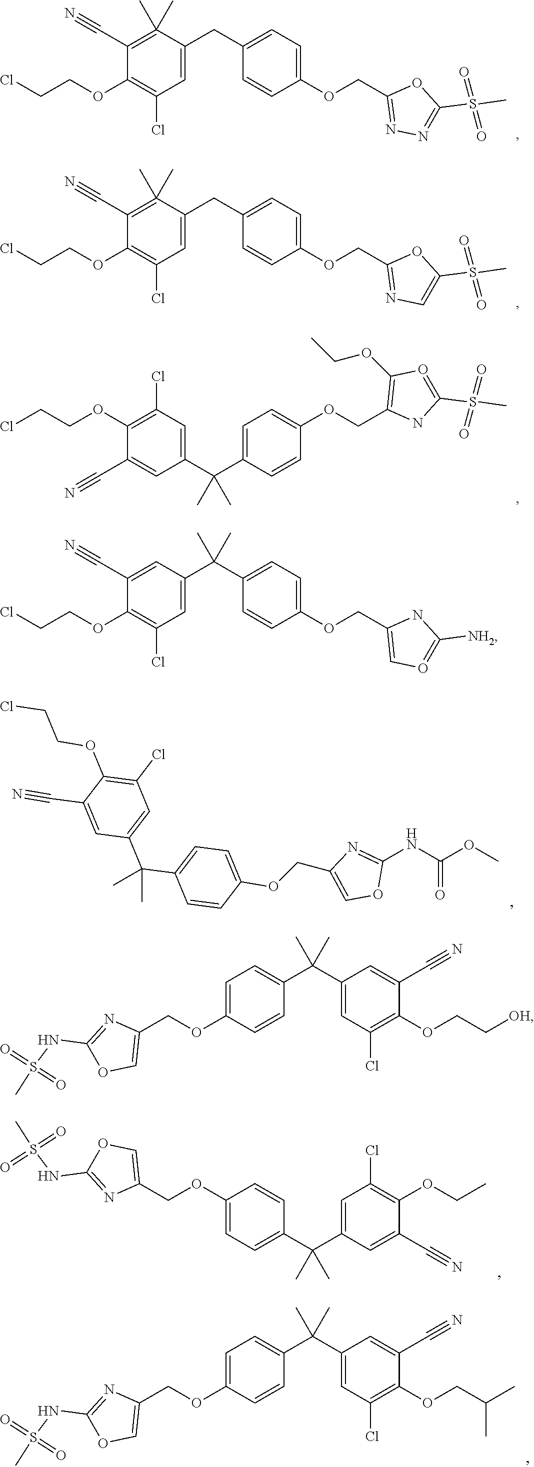 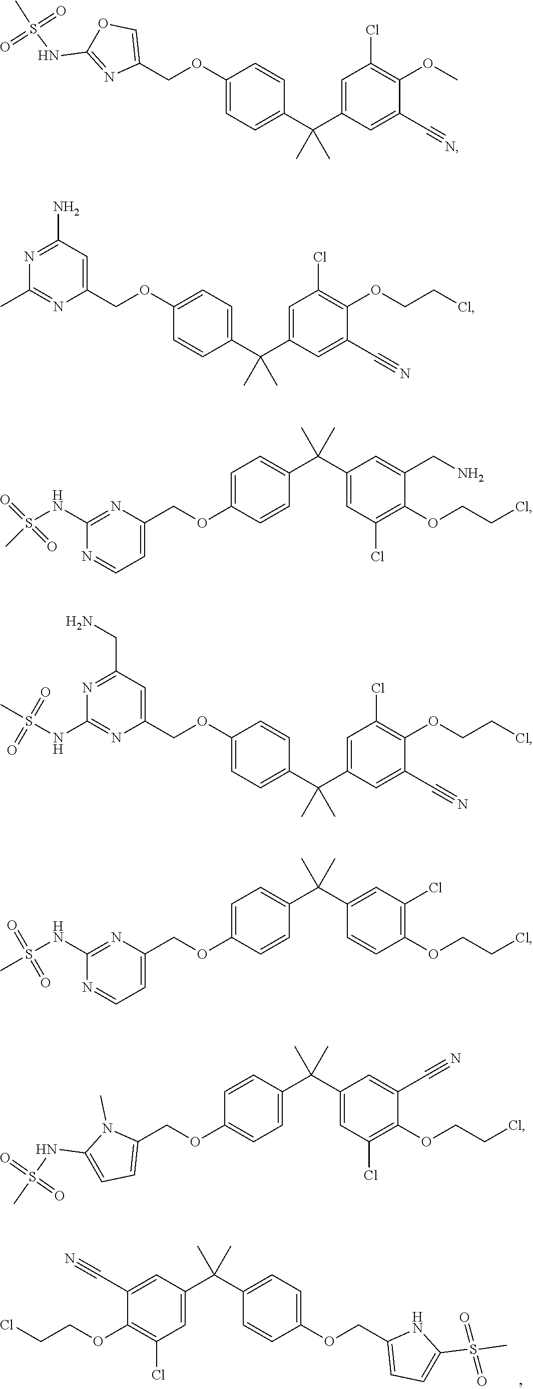 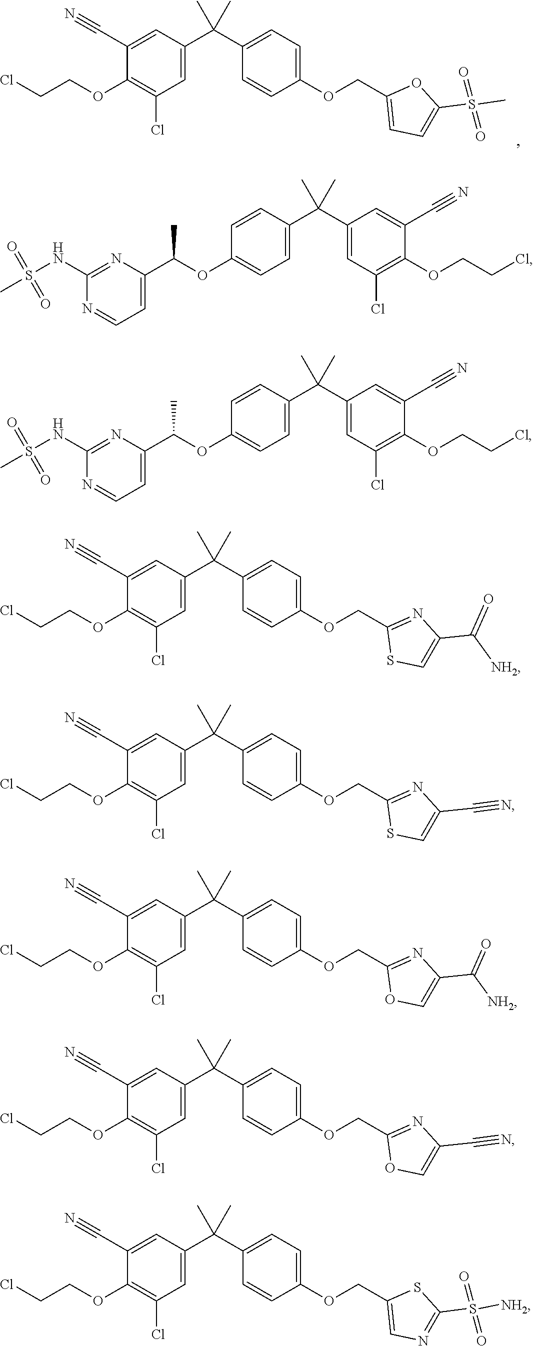 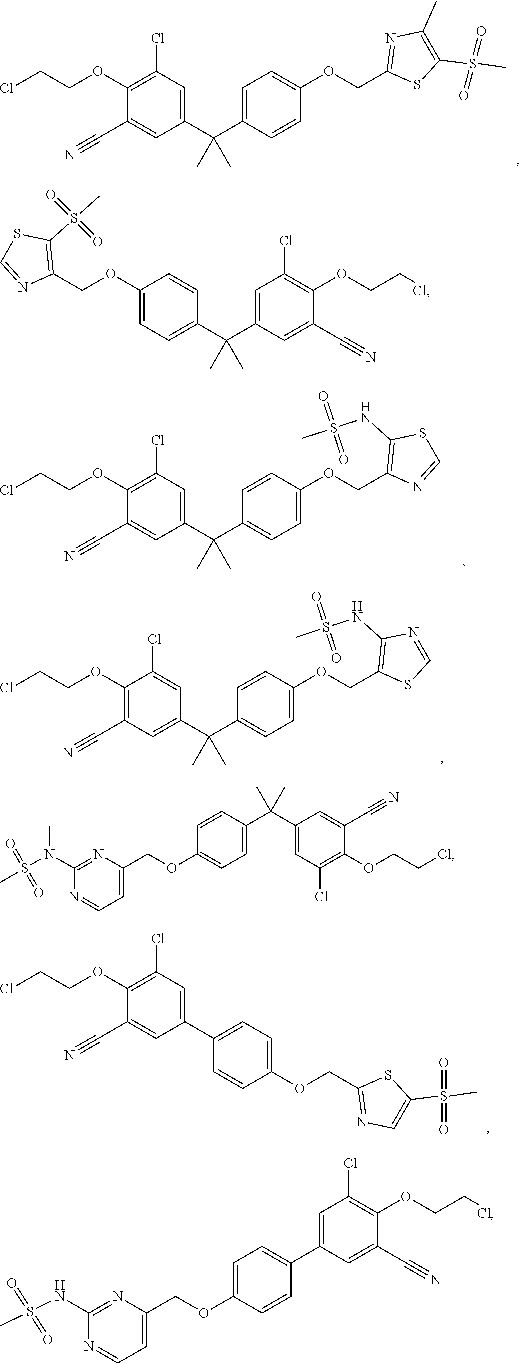 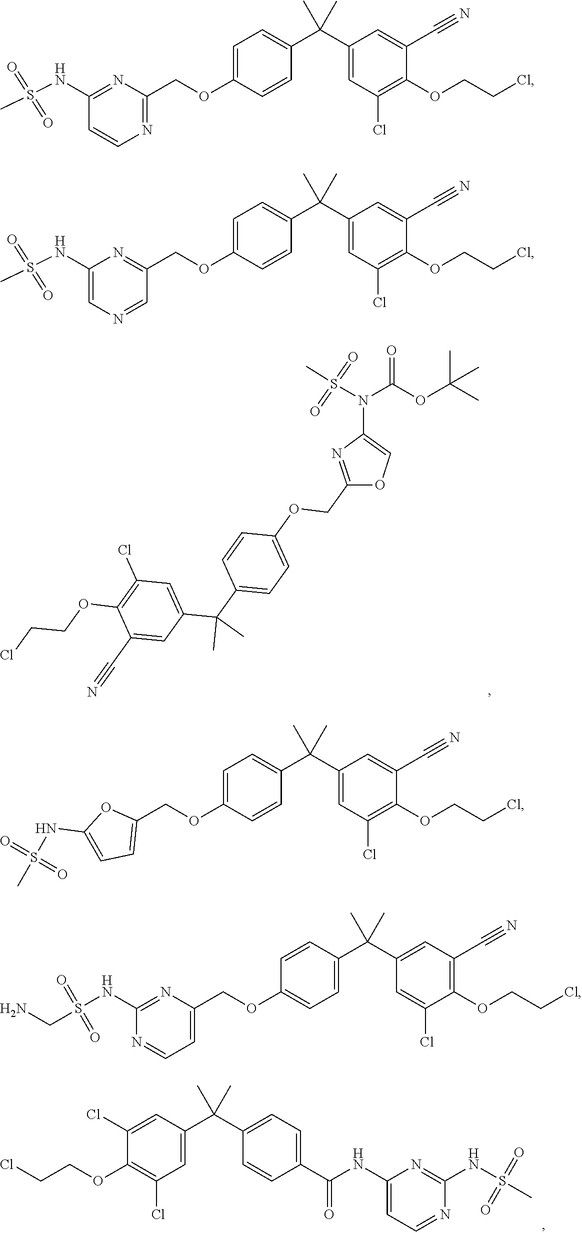 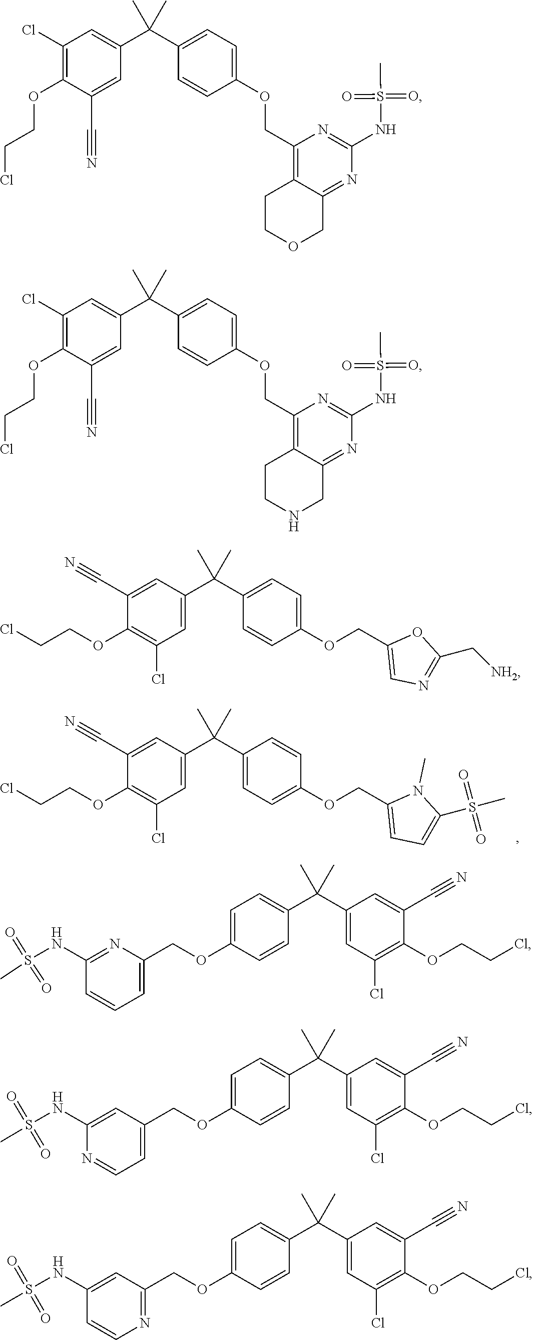 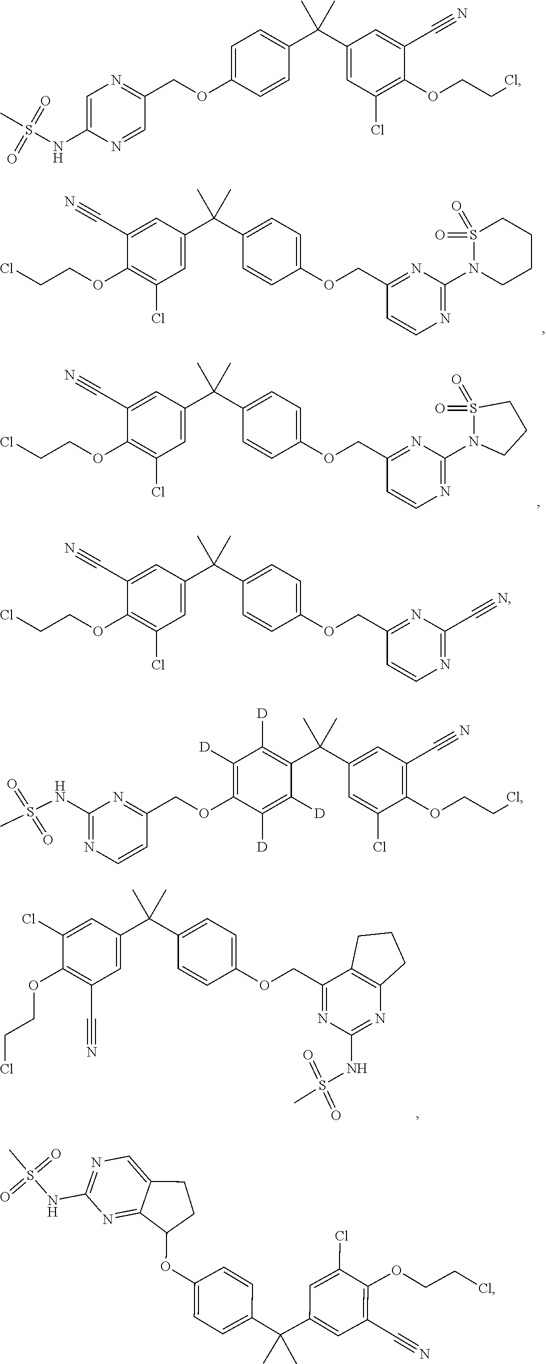 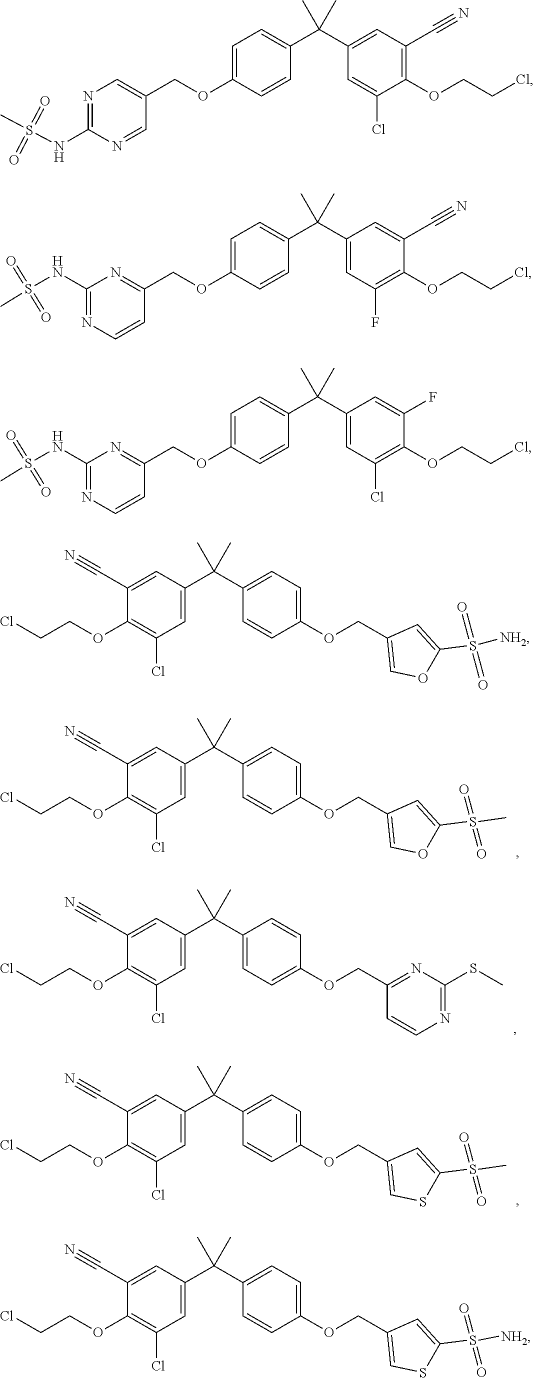 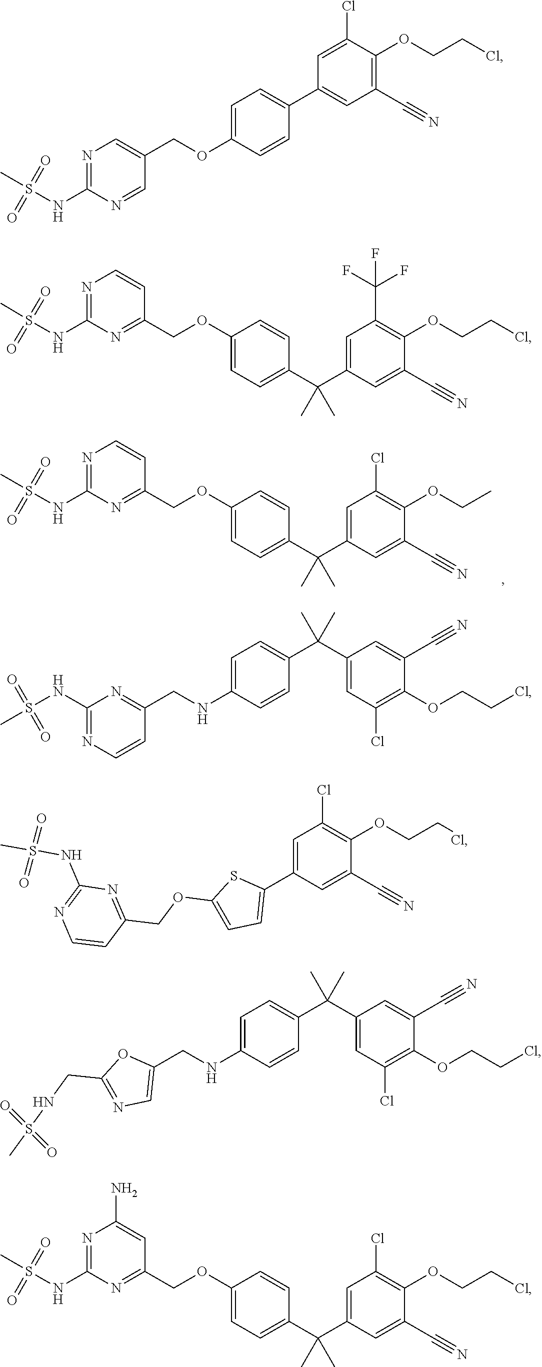 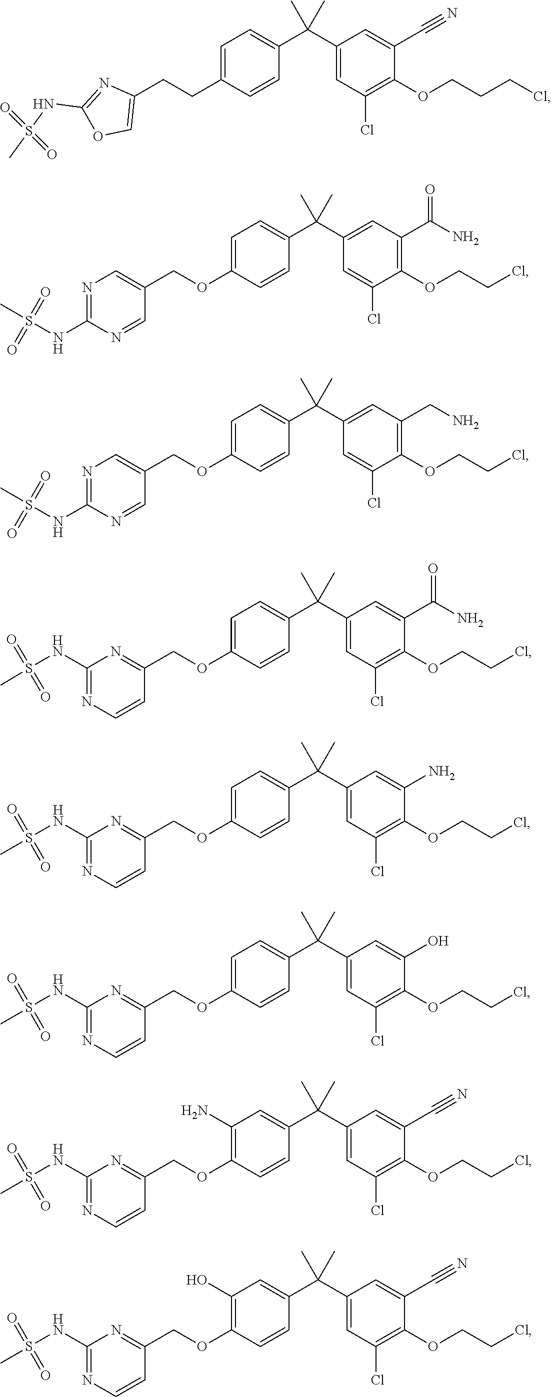 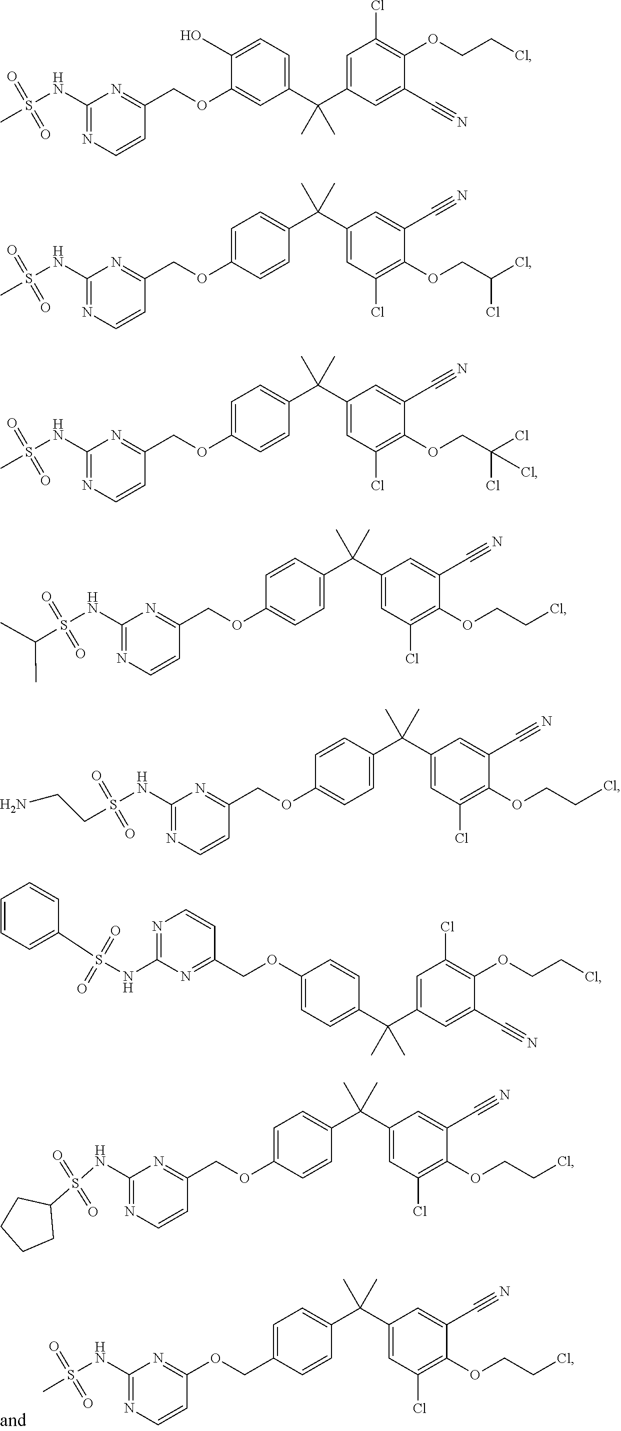
or a pharmaceutically acceptable salt, tautomer, stereoisomer or prodrug thereof; wherein one atom or one chemical group in the PTC is replaced to form a covalent bond to the LI.

46. The compound of claim 45, wherein the PTC is selected from the group consisting of: 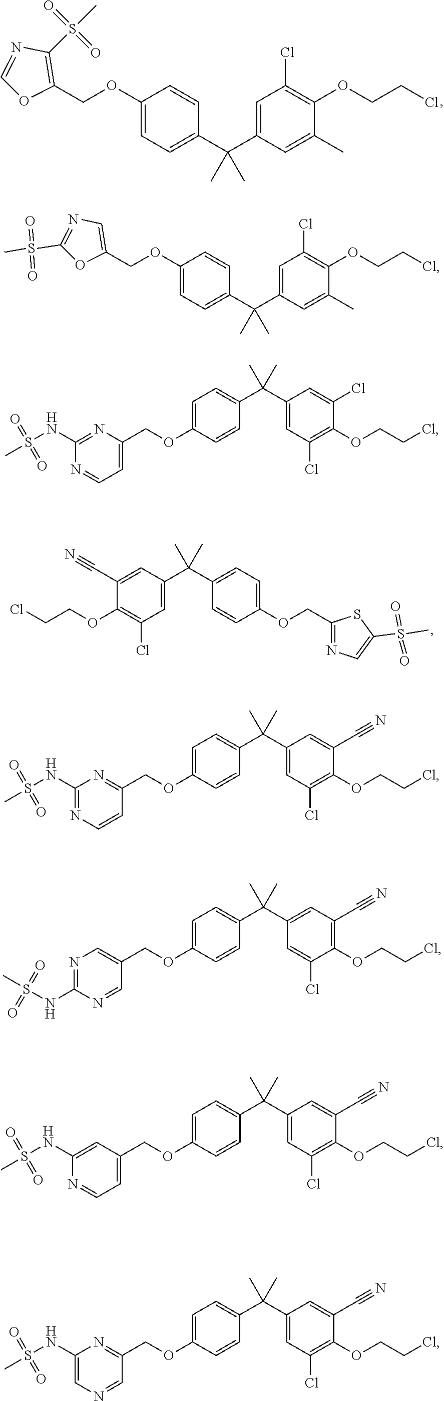 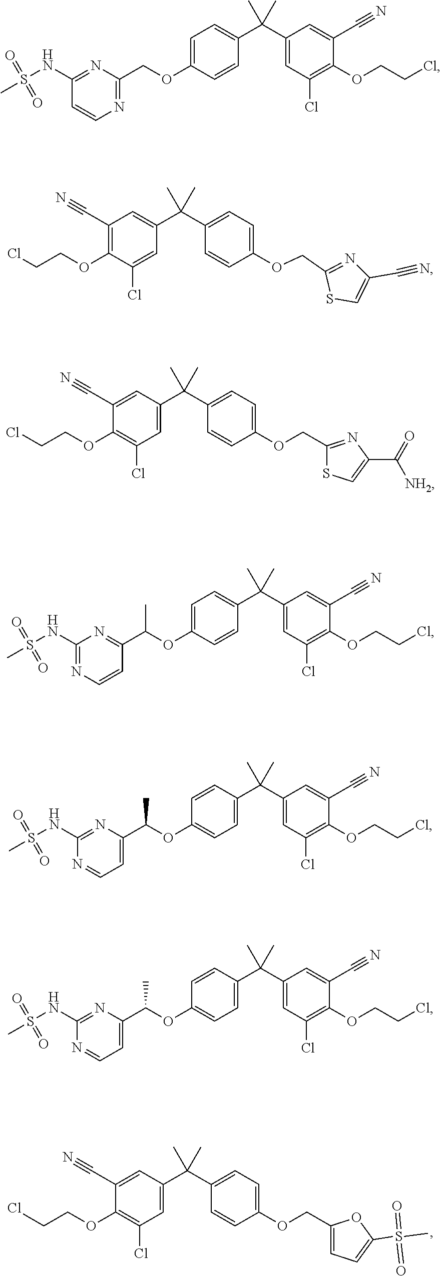 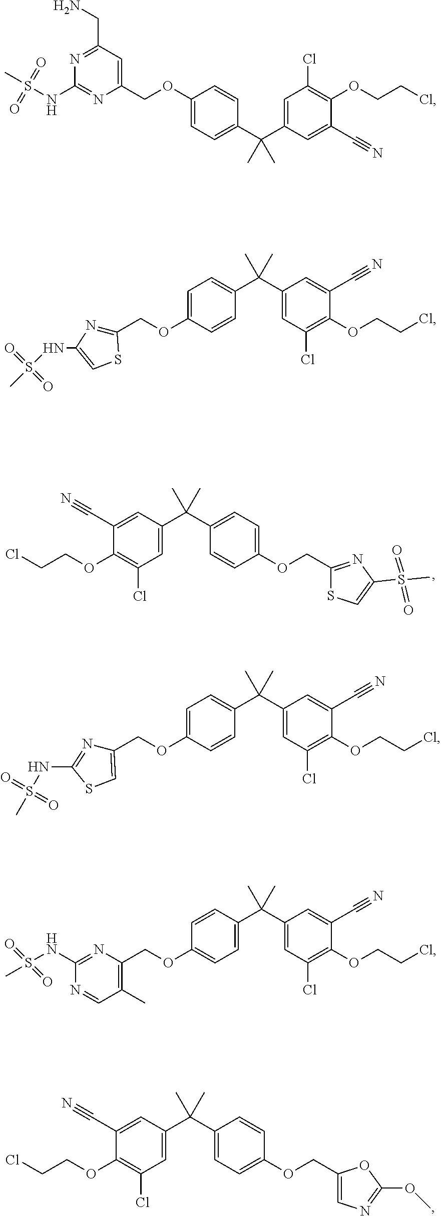 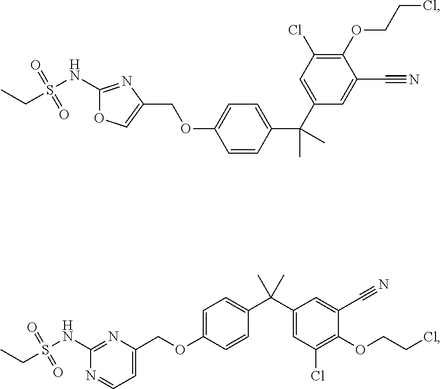 [SMILES: CCS(=O)(=O)Nc1nc(COc2ccc(C(C)(C)c3cc(Cl)c(OCCCl)c(C#N)c3)cc2)co1 ; CCS(=O)(=O)Nc1nccc(COc2ccc(C(C)(C)c3cc(Cl)c(OCCCl)c(C#N)c3)cc2)n1]
or a pharmaceutically acceptable salt, tautomer, stereoisomer or prodrug thereof and wherein one atom or one chemical group in the PTC is replaced to form a covalent bond to the LI.

47. The compound of claim 45 or 46, wherein a) a Cl atom is replaced with a covalent bond to the LI or b) a hydrogen atom is replaced with a covalent bond to the LI.

48. The compound of any one of the preceding claims, wherein the PTC is selected from:

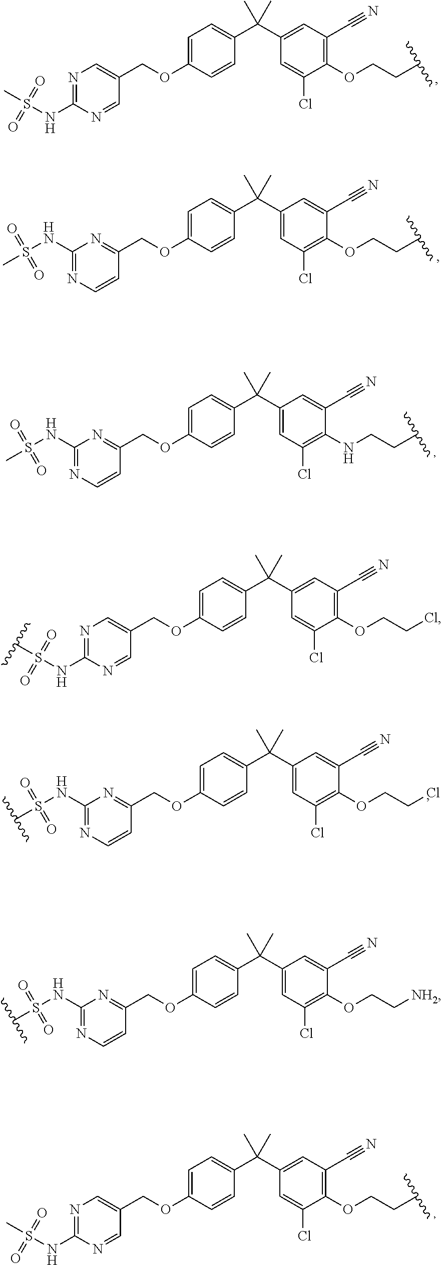

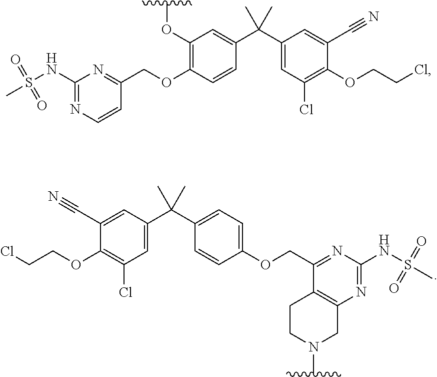 [SMILES: *Oc1cc(C(C)(C)c2cc(Cl)c(OCCCl)c(C#N)c2)ccc1OCc1ccnc(NS(C)(=O)=O)n1 ; *N1CCc2c(COc3ccc(C(C)(C)c4cc(Cl)c(OCCCl)c(C#N)c4)cc3)nc(NS(C)(=O)=O)nc2C1]

49. The compound of any one of claims 1-44, wherein the compound is a compound of formula (W-IV)L (W-IVA), (W-V), (W-VA), (W-VI), (W-VIA), (VII), (VIII), (IX) or (X): 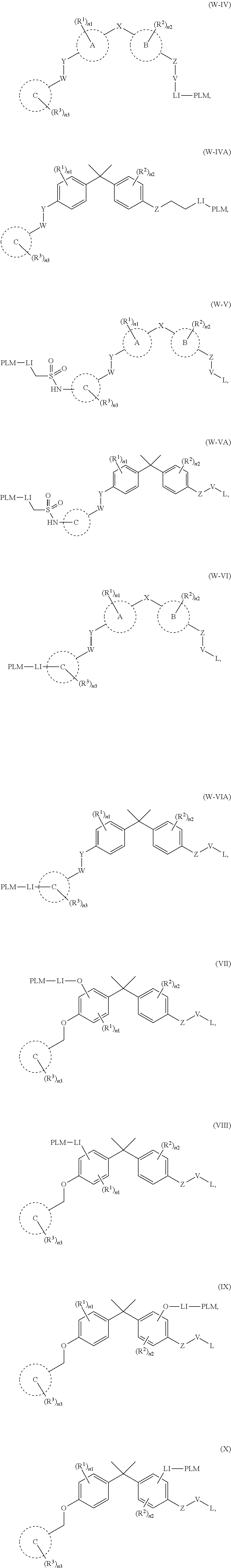
or a pharmaceutically acceptable salt thereof.

50. A compound selected from the group consisting of: 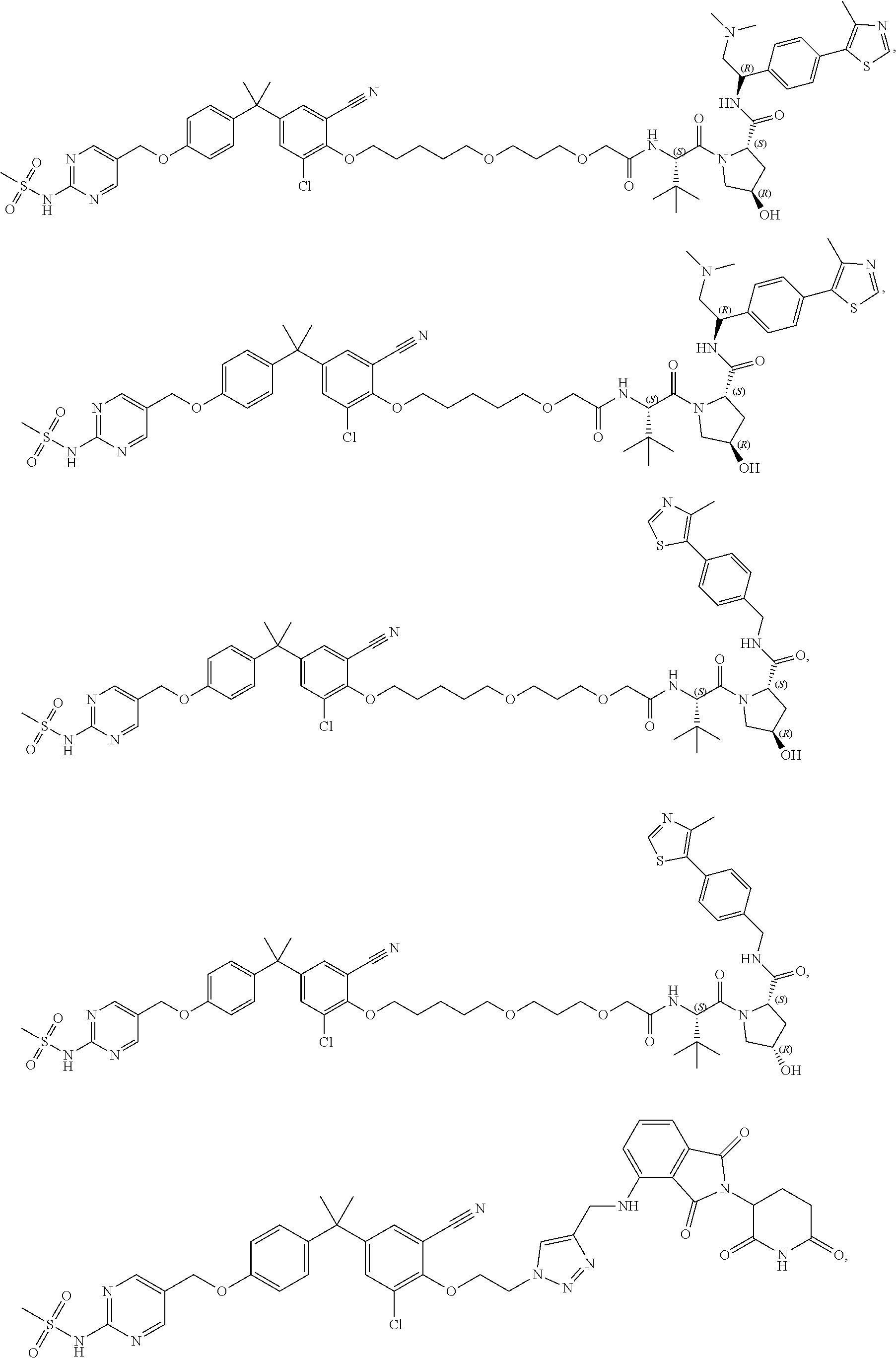 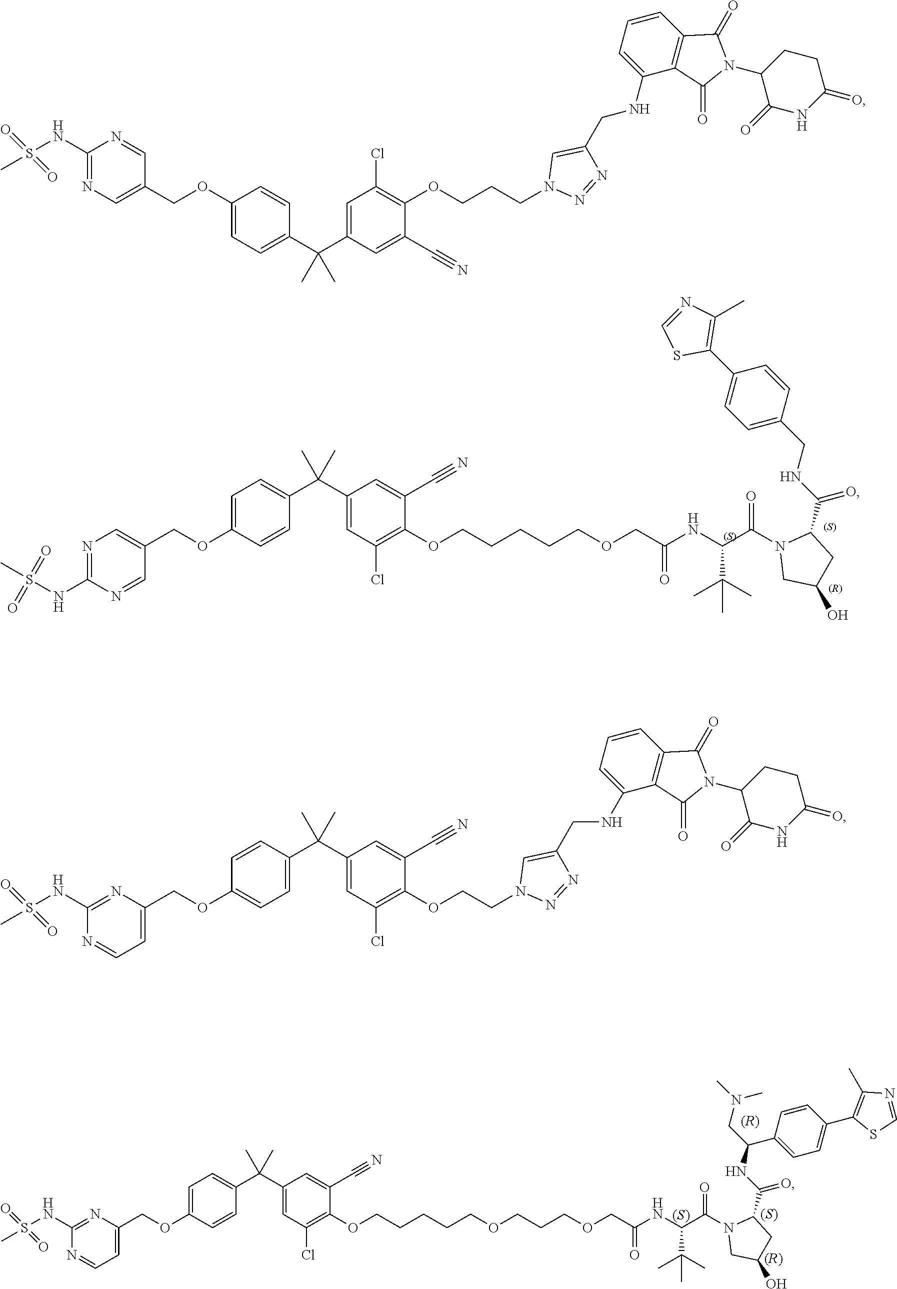 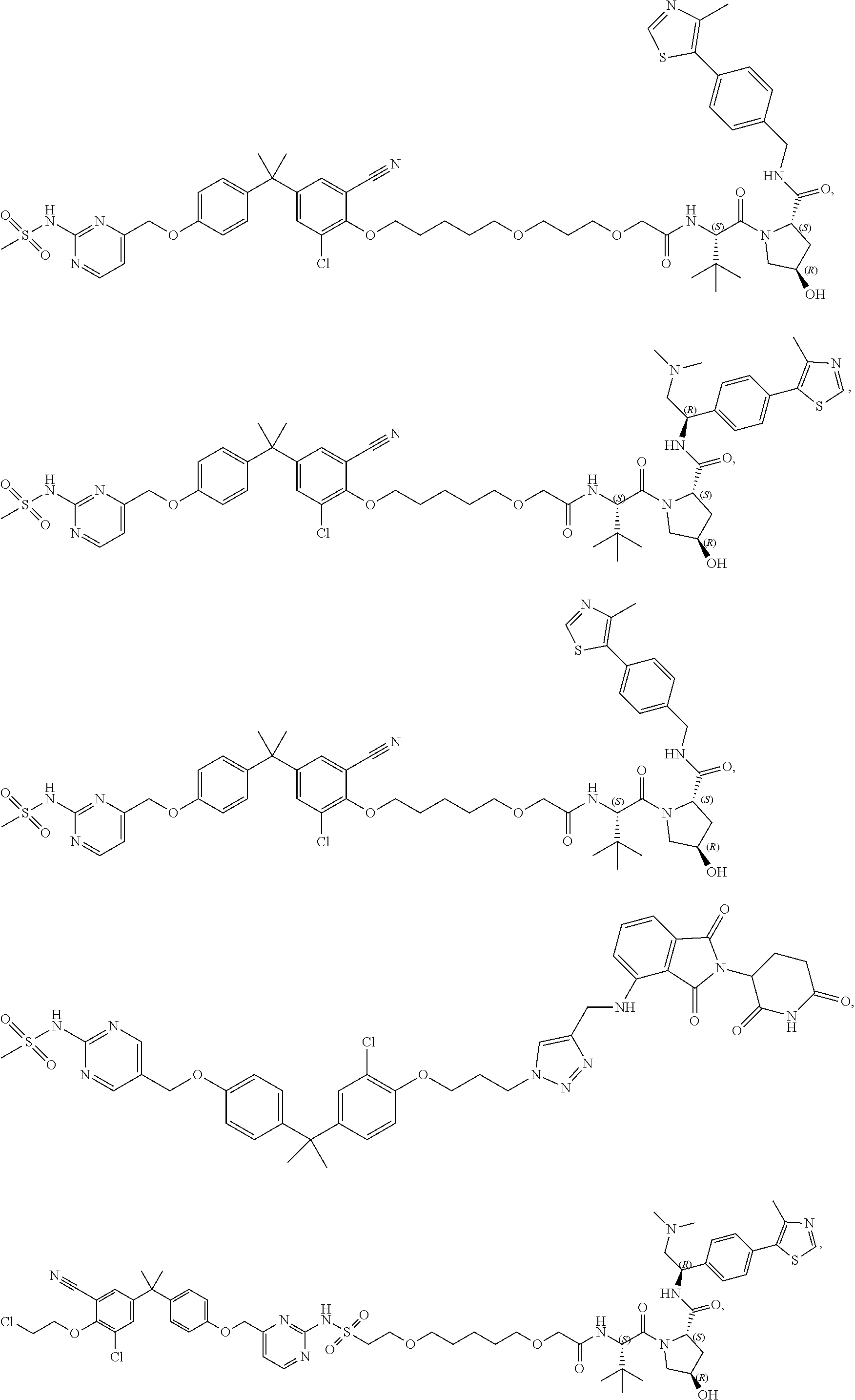 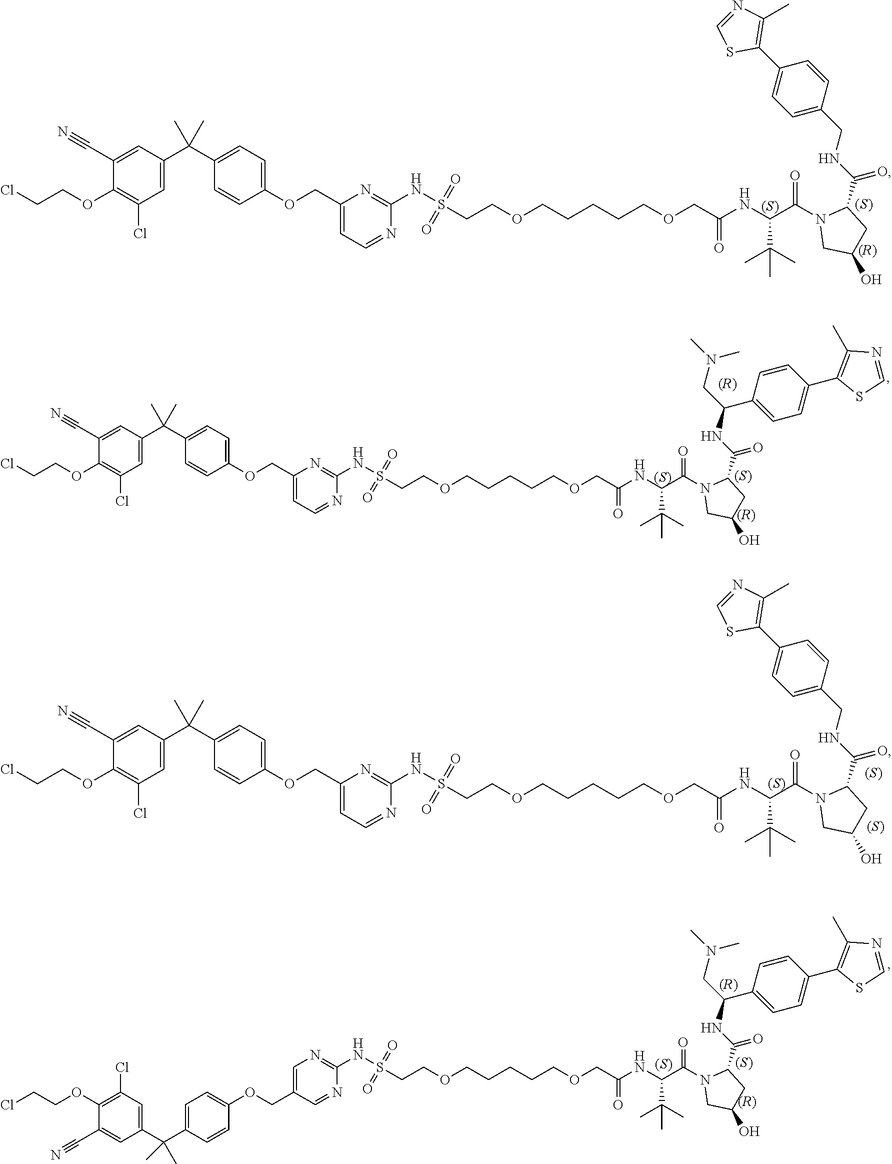 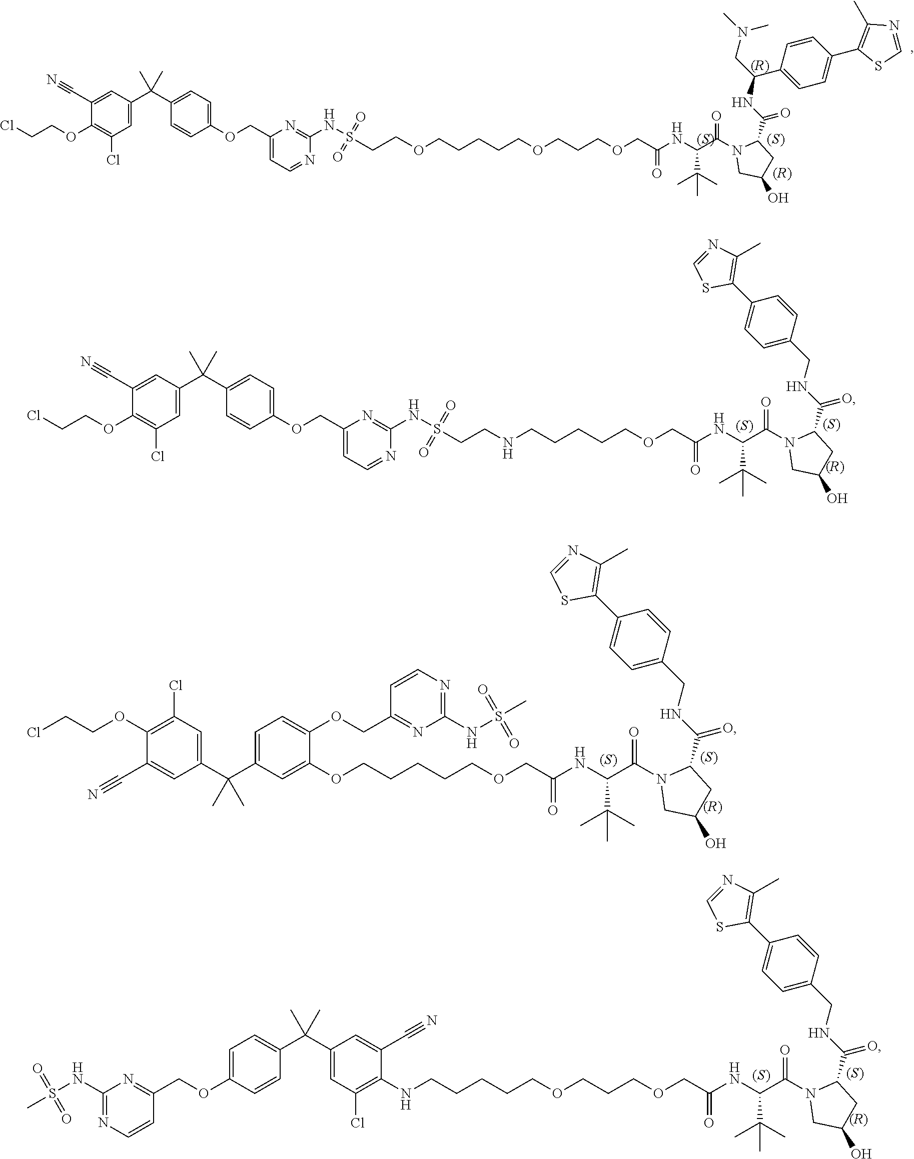 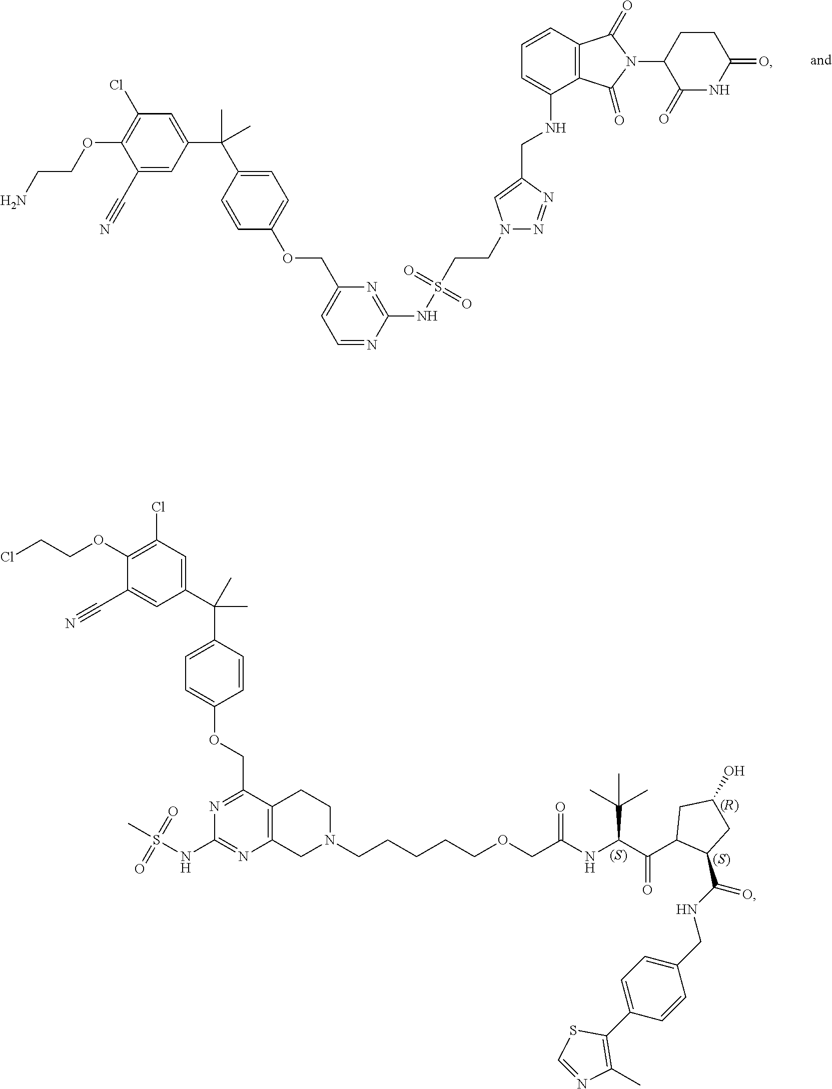
or a pharmaceutically acceptable salt thereof.

51. A pharmaceutical composition comprising a compound of any one of claims 1-50 and a pharmaceutically acceptable carrier.

52. A method for modulating androgen receptor activity, comprising administering a compound of any one of claims 1-50, to a subject in need thereof.

53. The method of claim 52, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

54. A method for treating cancer, comprising administering a compound of any one of claims 1-50, to a subject in need thereof.

55. A compound selected from: 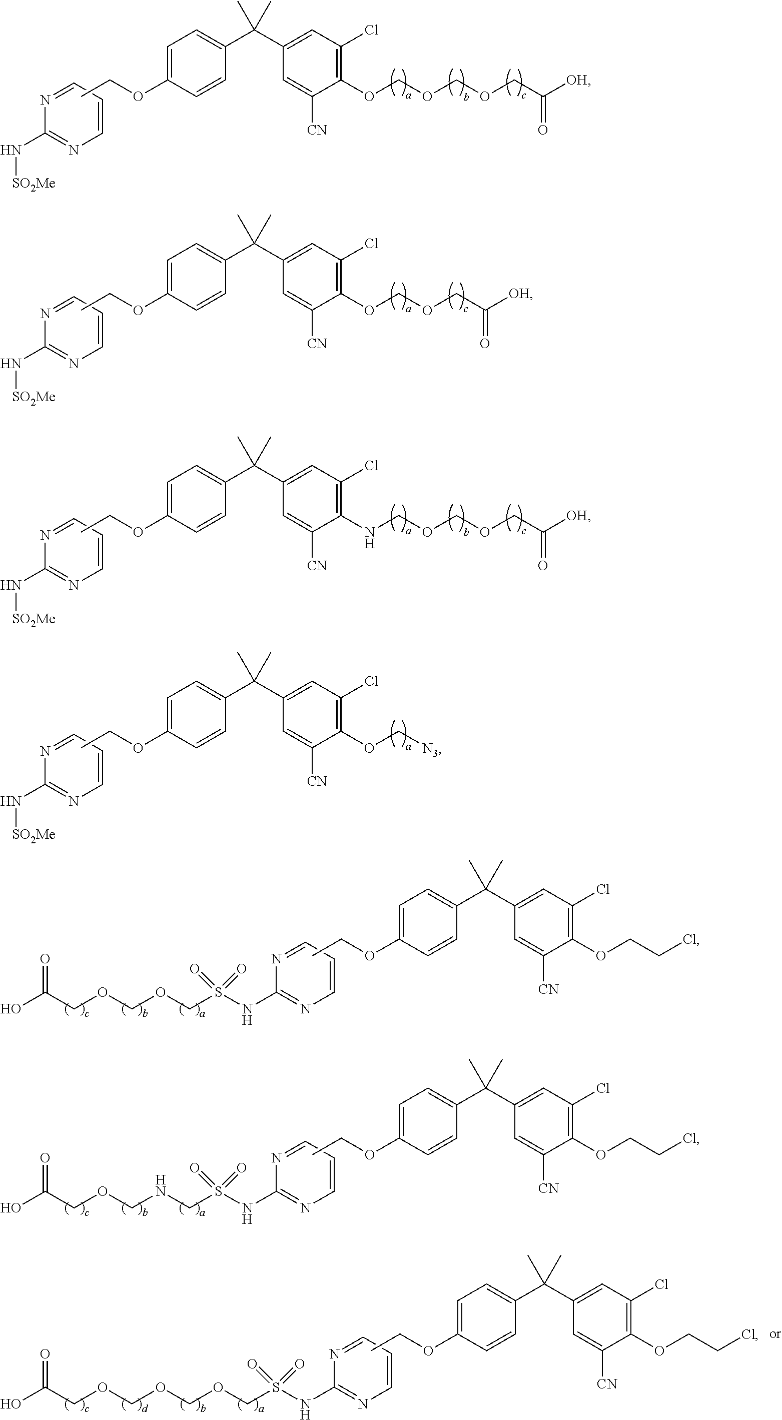 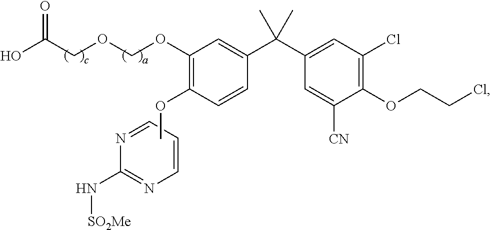
or a pharmaceutically acceptable salt thereof, wherein a, b, c, and d are each independently an integer between 1 to 10.
